(19) United States
(12) Patent Application Publication
(10) Pub. No.: US 2024/0116912 A1
(43) Pub. Date: Apr. 11, 2024

(54) COMPOUNDS AND METHODS FOR MODULATING FXR

(71) Applicant: Terns Pharmaceuticals, Inc., Foster City, CA (US)

(72) Inventors: Yingzi XU, Palo Alto, CA (US); Kevin KLUCHER, Foster City, CA (US); F. Anthony ROMERO, Redwood City, CA (US)

(21) Appl. No.: 18/259,888

(22) PCT Filed: Dec. 29, 2021
(86) PCT No.: PCT/US2021/073153

§ 371 Date: Jun. 29, 2023

Related U.S. Application Data
(60) Provisional application No. 63/132,363, filed on Dec. 30, 2020.

(51) Int. Cl. C07D 413/14; C07D 417/14; C07D 451/06

(52) U.S. Cl. CPC C07D 413/14; C07D 417/14; C07D 451/06

(57) ABSTRACT

Disclosed herein are compounds that can be used as Farnesoid X Receptor (FXR) agonists, compositions containing these compounds and methods of use thereof.

### CROSS-REFERENCE TO RELATED APPLICATIONS

This application claims priority to and the benefit of U.S. Provisional Application No. 63/132,363, filed on Dec. 30, 2020, the content of which is incorporated herein by reference in its entirety.

### FIELD

This disclosure generally relates to compounds for modulating farnesoid X receptor (FXR) and methods of use thereof.

### BACKGROUND

FXR agonists are ligands for a nuclear receptor that regulates the transcription of genes that control triglyceride, cholesterol, and carbohydrate metabolism. The above efforts and others not withstanding, there remains a need to discover and develop compounds that are believed to be potent and efficacious (based on in-vitro and in-vivo models) agonists of FXR. Such compounds would be useful for remedy or improvement of the lives of patients in need of treatment of liver disorders, such as liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

### BRIEF SUMMARY

Disclosed herein are compounds that can be used as Farnesoid X Receptor (FXR) agonists, compositions containing these compounds and methods for treating diseases or conditions mediated by FXR.

In one aspect, provided is a compound of Formula (I), or a stereoisomer, tautomer, or a pharmaceutically acceptable salt of any of the foregoing, as detailed herein.

Further provided is a pharmaceutical composition comprising a compound of Formula (I), or a stereoisomer, tautomer, or a pharmaceutically acceptable salt of any of the foregoing, and a pharmaceutically acceptable carrier or excipient.

In another aspect, provided is a method of treating a disease or a condition mediated by FXR in a subject in need thereof, comprising administering to the subject a therapeutically effective amount of a compound of Formula (I), or a stereoisomer, tautomer, or a pharmaceutically acceptable salt of any of the foregoing. In some embodiments, the disease or the condition is a liver disease. In some embodiments, the disease or disorder is liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), or non-alcoholic steatohepatitis (NASH). Also provided is a compound of Formula (I), or a stereoisomer, tautomer, or a pharmaceutically acceptable salt of any of the foregoing as detailed herein, for use in a method as disclosed herein. Also provided is use of a compound of Formula (I), or a stereoisomer, tautomer, or a pharmaceutically acceptable salt of any of the foregoing as detailed herein, in the manufacture of a medicament for treating a disease or disorder as disclosed herein.

Further provided is a kit comprising a compound of Formula (I), or a stereoisomer, tautomer, or a pharmaceutically acceptable salt of any of the foregoing. In some embodiments, the kit comprises instructions for use according to a method described herein.

In yet another aspect, provided is a method of making a compound of Formula (I), or a stereoisomer, tautomer, or a pharmaceutically acceptable salt of any of the foregoing. Also provided are compound intermediates useful in synthesis of a compound of Formula (I), or a stereoisomer, tautomer, or a pharmaceutically acceptable salt of any of the foregoing.

### DETAILED DESCRIPTION

### Definitions

As used herein, the following definitions shall apply unless otherwise indicated. Further, if any term or symbol used herein is not defined as set forth below, it shall have its ordinary meaning in the art.

As used herein and in the appended claims, the singular forms “a”, “an” and “the” include plural forms, unless the context clearly dictates otherwise.

As used herein, and unless otherwise specified, the terms “about” and “approximately,” when used in connection with doses, amounts, or weight percent of ingredients of a composition or a dosage form, mean a dose, amount, or weight percent that is recognized by those of ordinary skill in the art to provide a pharmacological effect equivalent to that obtained from the specified dose, amount, or weight percent. Specifically, the terms “about” and “approximately,” when used in connection with a value, contemplate a variation within ±15%, within ±10%, within ±5%, within ±4%, within ±3%, within ±2%, within +1%, or within ±0.5% of the specified value. Reference to “about” a value or parameter herein includes (and describes) embodiments that are directed to that value or parameter per se. For example, description referring to “about X” includes description of “X”.

“Comprising” is intended to mean that the compositions and methods include the recited elements, but not exclude others. “Consisting essentially of” when used to define compositions and methods, shall mean excluding other elements of any essential significance to the combination. For example, a composition consisting essentially of the elements as defined herein would not exclude other elements that do not materially affect the basic and novel characteristic(s) of the claimed invention. “Consisting of” shall mean excluding more than trace amount of, e.g., other ingredients and substantial method steps recited. Embodiments defined by each of these transition terms are within the scope of this disclosure.

“Combination therapy” or “combination treatment” refers to the use of two or more drugs or agents in treatment, e.g., the use of a compound of formula (I) as utilized herein together with another agent useful to treat liver disorders, such as NAFLD, NASH, and symptoms and manifestations of each thereof is a combination therapy. Administration in “combination” refers to the administration of two agents (e.g., a compound of formula (I) as utilized herein, and another agent) in any manner in which the pharmacological effects of both manifest in the patient at the same time. Thus, administration in combination does not require that a single pharmaceutical composition, the same dosage form, or even the same route of administration be used for administration of both agents or that the two agents be administered at precisely the same time. Both agent can also be formulated in a single pharmaceutically acceptable composition. A non-limiting example of such a single composition is an oral composition or an oral dosage form. For example, and without limitation, it is contemplated that a compound of formula (I) can be administered in combination therapy with another agent in accordance with the present disclosure.

The term “excipient” as used herein means an inert or inactive substance that may be used in the production of a drug or pharmaceutical, such as a tablet containing a compound of the disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“Pharmaceutically acceptable” refers to safe and non-toxic, preferably for in vivo, more preferably, for human administration.

“Pharmaceutically acceptable salt” refers to a salt that is pharmaceutically acceptable. A compound described herein may be administered as a pharmaceutically acceptable salt.

“Salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary and non-limiting cations useful in pharmaceutically acceptable salts include Na, K, Rb, Cs, NH4, Ca, Ba, imidazolium, and ammonium cations based on naturally occurring amino acids. When the compounds utilized herein contain basic functionality, such salts include, without limitation, salts of organic acids, such as carboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisulfate, mono-, di-, and tribasic phosphate, mesylate, tosylate, and the likes.

“Stereoisomer” or “stereoisomers” refer to compounds that differ in the stereogenicity of the constituent atoms such as, without limitation, in the chirality of one or more stereocenters or related to the cis or trans configuration of a carbon-carbon or carbon-nitrogen double bond. Stereoisomers include enantiomers and diastereomers.

As used herein, the term “subject” refers to an animal, including, but are not limited to, a primate (e.g., human), monkey, cow, pig, sheep, goat, horse, dog, cat, rabbit, rat, or mouse. The terms “subject” and “patient” are used interchangeably herein in reference, for example, to a mammalian subject, such as a human.

As used herein, “treatment” or “treating” is an approach for obtaining beneficial or desired results including clinical results. For purposes of this disclosure, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of this disclosure contemplate any one or more of these aspects of treatment.

“Therapeutically effective amount” or dose of a compound or a composition refers to that amount of the compound or the composition that results in reduction or inhibition of symptoms or a prolongation of survival in a patient. The results may require multiple doses of the compound or the composition.

“Alkyl” refers to monovalent saturated aliphatic hydrocarbyl groups having from 1 to 12 carbon atoms, preferably from 1 to 10 carbon atoms, and more preferably from 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). Cₓ alkyl refers to an alkyl group having x number of carbon atoms.

“Alkylene” refers to a divalent saturated aliphatic hydrocarbyl group having from 1 to 12 carbon atoms, preferably from 1 to 10 carbon atoms, and more preferably from 1 to 6 carbon atoms. This term includes, by way of example, linear and branched hydrocarbyl groups such as methylene (—CH₂—), ethylene (—CH₂CH₂— or —CH(Me)—), propylene (—CH₂CH₂CH₂— or —CH(Me)CH₂—, or —CH(Et)—) and the likes.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Aryl” refers to a monovalent aromatic carbocyclic group of from 6 to 14 carbon atoms having a single ring (e.g., phenyl (Ph)) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl. It is understood that “arylene” refers to a divalent aryl group as defined herein. For example, “phenylene” refers to a divalent phenyl group.

“Cyano” refers to the group —C≡N.

“Cycloalkyl” refers to saturated or unsaturated but nonaromatic cyclic alkyl groups of from 3 to 10 carbon atoms, preferably from 3 to 8 carbon atoms, and more preferably from 3 to 6 carbon atoms, having single or multiple cyclic rings including fused, bridged, and spiro ring systems. Cₓ cycloalkyl refers to a cycloalkyl group having x number of ring carbon atoms. Examples of suitable cycloalkyl groups include, for instance, adamantyl, cyclopropyl, cyclobutyl, cyclopentyl, and cyclooctyl. One or more the rings can be aryl, heteroaryl, or heterocyclic provided that the point of attachment is through the non-aromatic, non-heterocyclic ring saturated carbocyclic ring.

The term “dyslipidemia” as used herein refers to an abnormality in, or abnormal amounts of lipids and lipoproteins in the blood and the disease states resulting, caused by, exacerbated by, or adjunct to such abnormality (see Dorland's Illustrated Medical Dictionary, 29th edition, W.B Saunders publishing Company, New York, N.Y.). Disease states encompassed within the definition of dyslipidemia as used herein include hyperlipidemia, hypertriglyceremia, low plasma HDL, high plasma LDL, high plasma VLDL, liver cholestosis, and hypercholesterolemia.

The phrase “diseases related to dyslipidemia” as used herein refers to diseases including but not limited to atherosclerosis, thrombosis, coronary artery disease, stroke, and hypertension. Diseases related to dyslipidemia also include metabolic diseases such as obesity, diabetes, insulin resistance, and complications thereof. Complications of diabetes include but are not limited diabetic retinopathy.

“Halo” or “halogen” refers to fluoro, chloro, bromo and iodo and preferably is fluoro or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Preferred heteroaryls include 5 or 6 membered heteroaryls such as pyridinyl, pyrrolyl, thiophenyl, and furanyl. Other preferred heteroaryls include 9 or 10 membered heteroaryls, such as indolyl, quinolinyl, quinolonyl, isoquinolinyl, and isoquinolonyl. It is understood that “heteroarylene” refers to a divalent heteroaryl group as defined herein.

“Heterocycle” or “heterocyclic” or “heterocycloalkyl” or “heterocyclyl” refers to a saturated or partially saturated, but not aromatic, group having from 1 to 10 ring carbon atoms, preferably from 1 to 8 carbon atoms, and more preferably from 1 to 6 carbon atoms, and from 1 to 4 ring heteroatoms, preferably from 1 to 3 heteroatoms, and more preferably from 1 to 2 heteroatoms selected from the group consisting of nitrogen, sulfur, or oxygen. Cₓ heterocycloalkyl refers to a heterocycloalkyl group having x number of ring atoms including the ring heteroatoms. Heterocycle encompasses single ring or multiple condensed rings, including fused bridged and spiro ring systems. In fused ring systems, one or more the rings can be cycloalkyl, aryl or heteroaryl provided that the point of attachment is through the non-aromatic ring. In one embodiment, the nitrogen and/or sulfur atom(s) of the heterocyclic group are optionally oxidized to provide for the N-oxide, sulfinyl, sulfonyl moieties.

Examples of heterocyclyl and heteroaryl include, but are not limited to, azetidinyl, pyrrolyl, imidazolyl, pyrazolyl, pyridyl, pyrazyl, pyrimidyl, pyridazyl, indolizyl, isoindolyl, indolyl, dihydroindolyl, indazolyl, purinyl, quinolizinyl, isoquinolinyl, quinolinyl, phthalazinyl, naphthylpyridinyl, quinoxalinyl, quinazolinyl, cinnolinyl, pteridinyl, carbazolyl, carbolinyl, phenanthridinyl, acridinyl, phenanthrolinyl, isothiazolyl, phenazinyl, isoxazolyl, phenoxazinyl, phenothiazinyl, imidazolidinyl, imidazolinyl, piperidinyl, piperazinyl, indolinyl, phthalimidyl, 1,2,3,4-tetrahydroisoquinolinyl, 4,5,6,7-tetrahydrobenzo[b]thiophenyl, thiazolyl, thiazolidinyl, thiophenyl, benzo[b]thiophenyl, morpholinyl, thiomorpholinyl (also referred to as thiamorpholinyl), 1,1-dioxothiomorpholinyl, piperidinyl, pyrrolidinyl, and tetrahydrofuranyl.

“Oxo” refers to the atom (═O) or (O).

The terms “optional” or “optionally” as used throughout the specification means that the subsequently described event or circumstance may but need not occur, and that the description includes instances where the event or circumstance occurs and instances in which it does not. For example, “the nitrogen atom is optionally oxidized to provide for the N-oxide (N→O) moiety” means that the nitrogen atom may but need not be oxidized, and the description includes situations where the nitrogen atom is not oxidized and situations where the nitrogen atom is oxidized.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

It is understood that an optionally substituted moiety can be substituted with more than five substituents, if permitted by the number of valences available for substitution on the moiety. For example, a propyl group can be substituted with seven halogen atoms to provide a perhalopropyl group. The substituents may be the same or different.

### Compounds

In one aspect, provided is a compound of Formula (I):

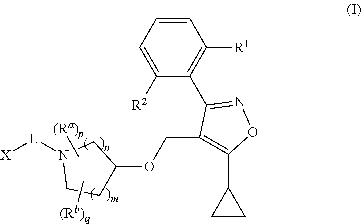

or a stereoisomer, tautomer, or a pharmaceutically acceptable salt of any of the foregoing,
wherein:
R¹ and R² are independently hydrogen, halogen, C₁-C₆ alkyl, or C₁-C₆ alkoxy, wherein the C₁-C₆ alkyl and C₁-C₆ alkoxy are optionally substituted by one to three halogen;
m is 0, 1 or 2;
n is 1 or 2;
p is 0, 1 or 2;
q is 0, 1 or 2;
R^{a} and R^{b} are independently halogen or C₁-C₆ alkyl,
   or p and q are both 1, and R^{a} and R^{b} are taken together with the carbon atoms to which they are attached to form a C₄-C₆ bridge;
L is —C(═O)—, phenylene, or 5- or 6-membered heteroarylene, wherein the phenylene and 5- or 6-membered heteroarylene are optionally substituted by one to three substituents each independently selected from the group consisting of C₁-C₆ alkyl, C₁-C₆ alkoxy, halogen, and cyano;
X is 3- to 6-membered heterocyclyl or 3- to 6-membered heteroaryl each containing one to four annular heteroatoms selected from the group consisting of N, O and S, wherein the 3- to 6-membered heterocyclyl and 3- to 6-membered heteroaryl are optionally substituted by one to three substituents each independently selected from the group consisting of halogen, cyano and oxo.

It is understood that every description, variation, embodiment or aspect of a moiety/variable may be combined with every description, variation, embodiment or aspect of other moieties/variables the same as if each and every combination of descriptions were specifically and individually listed. For example, every description, variation, embodiment or aspect provided herein with respect to R¹ of Formula (I), or any related formulae such as Formulae (II)-(IV), may be combined with every description, variation, embodiment or aspect of R², m, n, p, q, R^{a}, R^{b}, L and/or X the same as if each and every combination were specifically and individually listed.

In some embodiments, provided is a compound of Formula (II):

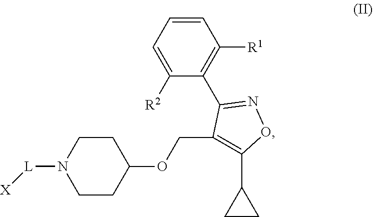

or a stereoisomer, tautomer, or a pharmaceutically acceptable salt of any of the foregoing, wherein R¹, R², L and X are as detailed herein for Formula (I).

In some embodiments, provided is a compound of Formula (III):

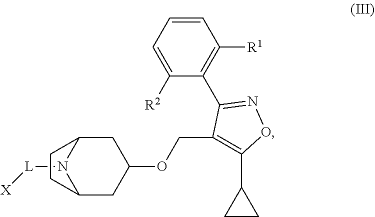

or a stereoisomer, tautomer, or a pharmaceutically acceptable salt of any of the foregoing, wherein R¹, R², L and X are as detailed herein for Formula (I).

In some embodiments, provided is a compound of Formula (IV):

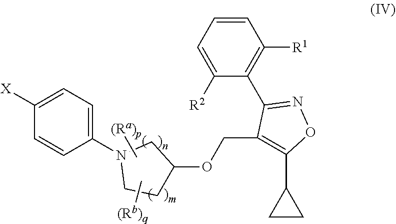

or a stereoisomer, tautomer, or a pharmaceutically acceptable salt of any of the foregoing, wherein R¹, R², R^{a}, R^{b}, m, n, p, q and X are as detailed herein for Formula (I).

In some embodiments of a compound of Formula (I) or any related formula where applicable, or a stereoisomer, tautomer, or a pharmaceutically acceptable salt of any of the foregoing, R¹ is hydrogen. In some embodiments, R¹ is halogen such as chloro or fluoro. In some embodiments, R¹ is chloro. In some embodiments, R¹ is fluoro. In some embodiments, R¹ is C₁-C₆ alkyl optionally substituted by one to three halogen, such as methyl or ethyl, each of which is optionally substituted by one to three halogen. In some embodiments, R¹ is methyl optionally substituted by one to three halogen. In some embodiments, R¹ is ethyl optionally substituted by one to three halogen. In some embodiments, R¹ is C₁-C₆ alkoxy optionally substituted by one to three halogen, such as methoxy or ethoxy, each of which is optionally substituted by one to three halogen. In some embodiments, R¹ is methoxy optionally substituted by one to three halogen. In some embodiments, R¹ is ethoxy optionally substituted by one to three halogen. In some embodiments, R¹ is hydrogen, chloro, fluoro, methoxy, ethoxy, or trifluoromethoxy. In some embodiments, R¹ is chloro or fluoro. In some embodiments, R¹ is methoxy, ethoxy, or trifluoromethoxy.

In some embodiments of a compound of Formula (I) or any related formula where applicable, or a stereoisomer, tautomer, or a pharmaceutically acceptable salt of any of the foregoing, R² is hydrogen. In some embodiments, R² is halogen such as chloro or fluoro. In some embodiments, R² is chloro. In some embodiments, R² is fluoro. In some embodiments, R² is C₁-C₆ alkyl optionally substituted by one to three halogen, such as methyl or ethyl, each of which is optionally substituted by one to three halogen. In some embodiments, R² is methyl optionally substituted by one to three halogen. In some embodiments, R² is ethyl optionally substituted by one to three halogen. In some embodiments, R² is C₁-C₆ alkoxy optionally substituted by one to three halogen, such as methoxy or ethoxy, each of which is optionally substituted by one to three halogen. In some embodiments, R² is methoxy optionally substituted by one to three halogen. In some embodiments, R² is ethoxy optionally substituted by one to three halogen. In some embodiments, R² is hydrogen, chloro, fluoro, methoxy, ethoxy, or trifluoromethoxy. In some embodiments, R² is chloro or fluoro. In some embodiments, R² is methoxy, ethoxy, or trifluoromethoxy.

In some embodiments of a compound of Formula (I) or any related formula where applicable, or a stereoisomer, tautomer, or a pharmaceutically acceptable salt of any of the foregoing, R¹ and R² are are independently hydrogen, halogen, C₁-C₆ alkyl, or C₁-C₆ alkoxy optionally substituted by one to three halogen. In some embodiments, R¹ and R² are independently hydrogen, chloro, fluoro, methoxy, ethoxy or trifluoromethoxy. In some embodiments, at least one of R¹ and R² is not hydrogen. In some embodiments, R¹ and R² are both halogen. In some embodiments, R¹ and R² are both chloro. In some embodiments, R¹ and R² are both fluoro. In some embodiments, one of R¹ and R² is hydrogen, chloro, fluoro, methoxy, ethoxy or trifluoromethoxy and the other is hydrogen.

In some embodiments of a compound of Formula (I) or any related formula where applicable, or a stereoisomer, tautomer, or a pharmaceutically acceptable salt of any of the foregoing, m is 0. In some embodiments, m is 1. In some embodiments, m is 2.

In some embodiments of a compound of Formula (I) or any related formula where applicable, or a stereoisomer, tautomer, or a pharmaceutically acceptable salt of any of the foregoing, n is 1. In some embodiments, n is 2. In some embodiments, m is 0 and n is 1. In some embodiments, m is 0 and n is 2. In some embodiments, m is 1 and n is 1. In some embodiments, m is 1 and n is 2. In some embodiments, m is 2 and n is 1. In some embodiments, m is 2 and n is 2.

In some embodiments of a compound of Formula (I) or any related formula where applicable, or a stereoisomer, tautomer, or a pharmaceutically acceptable salt of any of the foregoing, p is 0. In some embodiments, p is 1. In some embodiments, p is 2.

In some embodiments of a compound of Formula (I) or any related formula where applicable, or a stereoisomer, tautomer, or a pharmaceutically acceptable salt of any of the foregoing, q is 0. In some embodiments, q is 1. In some embodiments, q is 2. In some embodiments, p and q are both 0. In some embodiments, p and q are both 1. In some embodiments, p and q are both 2. In some embodiments, one of p and q is 0, and the other is 1. In some embodiments, one of p and q is 0, and the other is 2.

In some embodiments of a compound of Formula (I) or any related formula where applicable, or a stereoisomer, tautomer, or a pharmaceutically acceptable salt of any of the foregoing, each R^{a} is independently a halogen such as chloro or fluoro. In some embodiments, each R^{a} is independently a C₁-C₆ alkyl such as methyl or ethyl. In some embodiments, each R^{a} is independently chloro, fluoro, or methyl.

In some embodiments of a compound of Formula (I) or any related formula where applicable, or a stereoisomer, tautomer, or a pharmaceutically acceptable salt of any of the foregoing, each R^{b} is independently a halogen such as chloro or fluoro. In some embodiments, each R^{b} is independently a C₁-C₆ alkyl such as methyl or ethyl. In some embodiments, each R^{b} is independently chloro, fluoro or methyl. In some embodiments, R^{a} and R^{b} are independently halogen. In some embodiments, R^{a} and R^{b} are independently C₁-C₆ alkyl. In some embodiments, R^{a} and R^{b} are independently chloro, fluoro, or methyl.

In some embodiments of a compound of Formula (I) or any related formula where applicable, or a stereoisomer, tautomer, or a pharmaceutically acceptable salt of any of the foregoing, p and q are both 1, and R^{a} and R^{b} are taken together with the carbon atoms to which they are attached to form a C₄-C₆ bridge. In some embodiments, p and q are both 1, and R^{a} and R^{b} are taken together with the carbon atoms to which they are attached to form a C₄ bridge such as

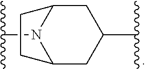 [SMILES: *C1CC2CCC(C1)N2*]

In some embodiments, R^{a} and R^{b} are taken together with the carbon atoms to which they are attached to form a C bridge. In some embodiments, R^{a} and R^{b} are taken together with the carbon atoms to which they are attached to form a C₆ bridge.

In some embodiments of a compound of Formula (I) or any related formula where applicable, or a stereoisomer, tautomer, or a pharmaceutically acceptable salt of any of the foregoing, L is —C(═O)—. In some embodiments, L is is phenylene or 5- or 6-membered heteroarylene, and wherein the phenylene and 5- or 6-membered heteroarylene are optionally substituted by one to three substituents each independently selected from the group consisting of C₁-C₆ alkyl, C₁-C₆ alkoxy, halogen, and cyano. In some embodiments, L is is phenylene or 5- or 6-membered heteroarylene, wherein the phenylene and 5- or 6-membered heteroarylene are optionally substituted by one to three substituents each independently selected from the group consisting of methyl, chloro and fluoro. In some embodiments, L is is phenylene or 5- or 6-membered heteroarylene. In some embodiments, L is phenylene optionally substituted by one to three substituents each independently selected from the group consisting of C₁-C₆ alkyl, C₁-C₆ alkoxy, halogen, and cyano. In some embodiments, L is phenylene optionally substituted by one to three substituents each independently selected from the group consisting of methyl, chloro and fluoro. In some embodiments, L is 5- or 6-membered heteroarylene optionally substituted by one to three substituents each independently selected from the group consisting of C₁-C₆ alkyl, C₁-C₆ alkoxy, halogen, and cyano. In some embodiments, L is 5-membered heteroarylene optionally substituted by one to three substituents each independently selected from the group consisting of C₁-C₆ alkyl, C₁-C₆ alkoxy, halogen, and cyano. In some embodiments, L is 6-membered heteroarylene optionally substituted by one to three substituents each independently selected from the group consisting of C₁-C₆ alkyl, C₁-C₆ alkoxy, halogen, and cyano. In some embodiments, L is 5- or 6-membered heteroarylene, optionally substituted by one to three substituents each independently selected from the group consisting of methyl, chloro and fluoro. In some embodiments, L is

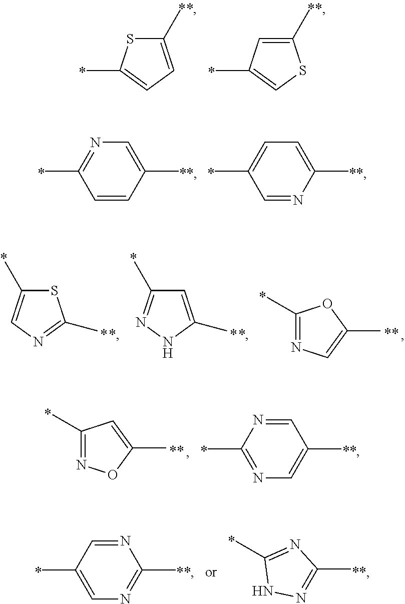

each of which is optionally substituted by one to three substituents each independently selected from the group consisting of C₁-C₆ alkyl, C₁-C₆ alkoxy, halogen, and cyano, wherein * represents the point of attachment to the remainder of the molecule via the nitrogen, and * * represents the point of attachment to the X moiety. In some embodiments, L is

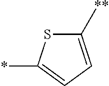

optionally substituted by one to three substituents each independently selected from the group consisting of C₁-C₆ alkyl, C₁-C₆ alkoxy, halogen, and cyano. In some embodiments, L is

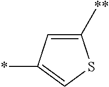

optionally substituted by one to three substituents each independently selected from the group consisting of C₁-C₆ alkyl, C₁-C₆ alkoxy, halogen, and cyano. In some embodiments, L is

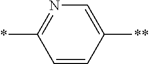

optionally substituted by one to three substituents each independently selected from the group consisting of C₁-C₆ alkyl, C₁-C₆ alkoxy, halogen, and cyano. In some embodiments, L is

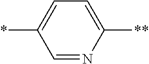

optionally substituted by one to three substituents each independently selected from the group consisting of C₁-C₆ alkyl, C₁-C₆ alkoxy, halogen, and cyano. In some embodiments, L is

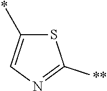

optionally substituted by one to three substituents each independently selected from the group consisting of C₁-C₆ alkyl, C₁-C₆ alkoxy, halogen, and cyano. In some embodiments, L is

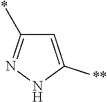

optionally substituted by one to three substituents each independently selected from the group consisting of C₁-C₆ alkyl, C₁-C₆ alkoxy, halogen, and cyano. In some embodiments, L is

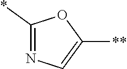

optionally substituted by one to three substituents each independently selected from the group consisting of C₁-C₆ alkyl, C₁-C₆ alkoxy, halogen, and cyano. In some embodiments, L is

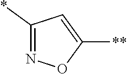

optionally substituted by one to three substituents each independently selected from the group consisting of C₁-C₆ alkyl, C₁-C₆ alkoxy, halogen, and cyano. In some embodiments, L is

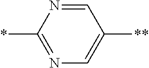

optionally substituted by one to three substituents each independently selected from the group consisting of C₁-C₆ alkyl, C₁-C₆ alkoxy, halogen, and cyano. In some embodiments, L is

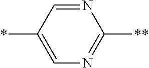

optionally substituted by one to three substituents each independently selected from the group consisting of C₁-C₆ alkyl, C₁-C₆ alkoxy, halogen, and cyano. In some embodiments, L is

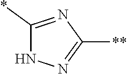

optionally substituted by one to three substituents each independently selected from the group consisting of C₁-C₆ alkyl, C₁-C₆ alkoxy, halogen, and cyano.

In some embodiments of a compound of Formula (I) or any related formula where applicable, or a stereoisomer, tautomer, or a pharmaceutically acceptable salt of any of the foregoing, X is 3- to 6-membered heterocyclyl or 3- to 6-membered heteroaryl each containing 2 or 3 annular heteroatoms selected from the group consisting of N and 0, wherein the 3- to 6-membered heterocyclyl and 3- to 6-membered heteroaryl are optionally substituted by one to three substituents each independently selected from the group consisting of cyano and oxo. In some embodiments, X is 3- to 6-membered heterocyclyl containing 2 or 3 annular heteroatoms selected from the group consisting of N, O, and S, wherein the 3- to 6-membered heterocyclyl is optionally substituted by one to three substituents each independently selected from the group consisting of cyano and oxo. In some embodiments, X is 5- or 6-membered heteroaryl containing 2 or 3 annular heteroatoms selected from the group consisting of N, O, and S, wherein the 5- or 6-membered heteroaryl is optionally substituted by one to three cyano. In some embodiments, X is

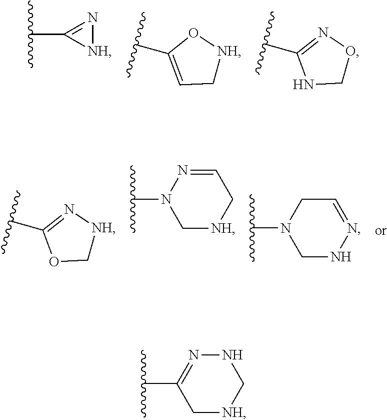

each of which is optionally substituted by one to three substituents each independently selected from the group consisting of cyano and oxo. In some embodiments, X is

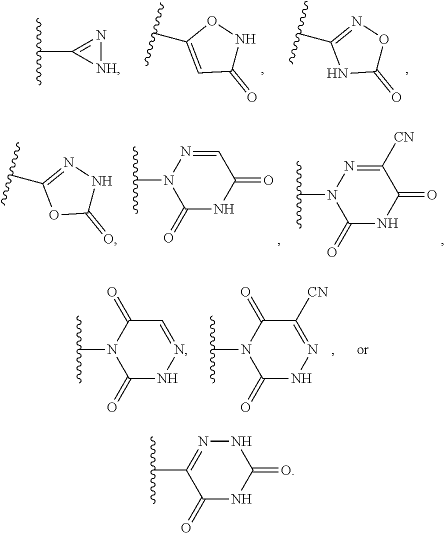

Representative compounds as listed in Table 1 below. In some embodiments, provided is a compound selected from the compounds in Table 1, or a stereoisomer, tautomer, or a pharmaceutically acceptable salt of any of the foregoing. In some embodiments, provided is a compound selected from the compounds in Table 1 or a pharmaceutically acceptable salt thereof.

TABLE 1

| Cmpd | | |
|---|---|---|
| No. | Structure | Name |
| 1 | 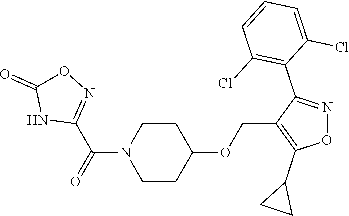 [SMILES: O=C(c1noc(=O)[nH]1)N1CCC(OCc2c(-c3c(Cl)cccc3Cl)noc2C2CC2)CC1] | 3-(4-((5-cyclopropyl-3-(2,6- dichlorophenyl)isoxazol-4- yl)methoxy)piperidine-1-carbonyl)- 1,2,4-oxadiazol-5(4H)-one |
| 2 | 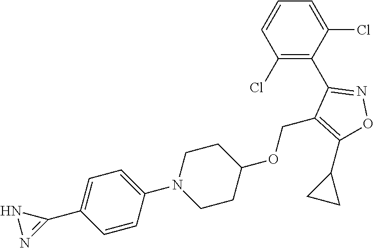 [SMILES: Clc1cccc(Cl)c1-c1noc(C2CC2)c1COC1CCN(c2ccc(C3=NN3)cc2)CC1] | 4-(((1-(4-(1H-diazirin-3- yl)phenyl)piperidin-4-yl)oxy)methyl)- 5-cyclopropyl-3-(2,6- dichlorophenyl)isoxazole |
| 3 | 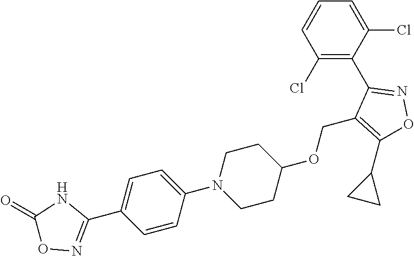 [SMILES: O=c1[nH]c(-c2ccc(N3CCC(OCc4c(-c5c(Cl)cccc5Cl)noc4C4CC4)CC3)cc2)no1] | 3-(4-(4-((5-cyclopropyl-3-(2,6- dichlorophenyl)isoxazol-4- yl)methoxy)piperidin-1-yl)phenyl)- 1,2,4-oxadiazol-5(4H)-one |
| 4 | 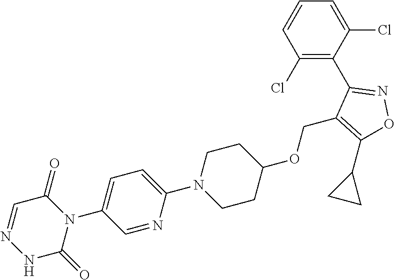 [SMILES: O=c1cn[nH]c(=O)n1-c1ccc(N2CCC(OCc3c(-c4c(Cl)cccc4Cl)noc3C3CC3)CC2)nc1] | 4-(6-(4-((5-cyclopropyl-3-(2,6- dichlorophenyl)isoxazol-4- yl)methoxy)piperidin-1-yl)pyridin-3- yl)-1,2,4-triazine-3,5(2H,4H)-dione |
| 5 | 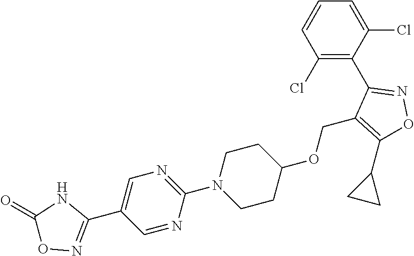 [SMILES: O=c1[nH]c(-c2cnc(N3CCC(OCc4c(-c5c(Cl)cccc5Cl)noc4C4CC4)CC3)nc2)no1] | 3-(2-(4-((5-cyclopropyl-3-(2,6- dichlorophenyl)isoxazol-4- yl)methoxy)piperidin-1-yl)pyrimidin-5- yl)-1,2,4-oxadiazol-5(4H)-one |
| 6 | 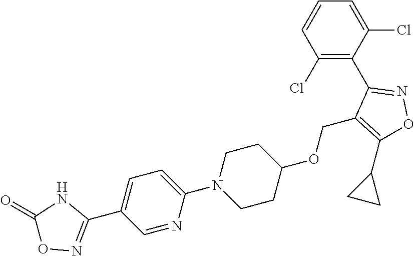 [SMILES: O=c1[nH]c(-c2ccc(N3CCC(OCc4c(-c5c(Cl)cccc5Cl)noc4C4CC4)CC3)nc2)no1] | 3-(6-(4-((5-cyclopropyl-3-(2,6- dichlorophenyl)isoxazol-4- yl)methoxy)piperidin-1-yl)pyridin-3- yl)-1,2,4-oxadiazol-5(4H)-one |
| 7 | 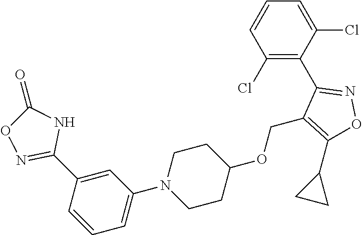 [SMILES: O=c1[nH]c(-c2cccc(N3CCC(OCc4c(-c5c(Cl)cccc5Cl)noc4C4CC4)CC3)c2)no1] | 3-(3-(4-((5-cyclopropyl-3-(2,6- dichlorophenyl)isoxazol-4- yl)methoxy)piperidin-1-yl)phenyl)- 1,2,4-oxadiazol-5(4H)-one |
| 8 | 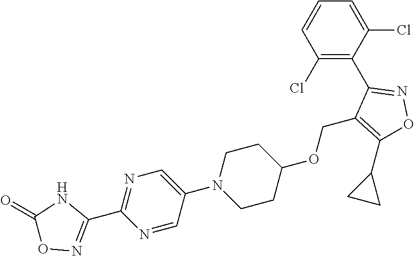 [SMILES: O=c1[nH]c(-c2ncc(N3CCC(OCc4c(-c5c(Cl)cccc5Cl)noc4C4CC4)CC3)cn2)no1] | 3-(5-(4-((5-cyclopropyl-3-(2,6- dichlorophenyl)isoxazol-4- yl)methoxy)piperidin-1-yl)pyrimidin-2- yl)-1,2,4-oxadiazol-5(4H)-one |
| 9 | 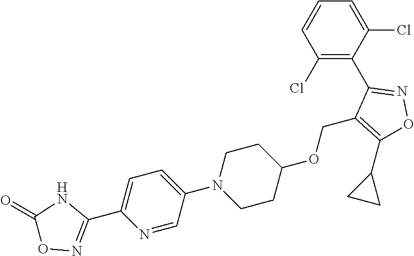 [SMILES: O=c1[nH]c(-c2ccc(N3CCC(OCc4c(-c5c(Cl)cccc5Cl)noc4C4CC4)CC3)cn2)no1] | 3-(5-(4-((5-cyclopropyl-3-(2,6- dichlorophenyl)isoxazol-4- yl)methoxy)piperidin-1-yl)pyridin-2- yl)-1,2,4-oxadiazol-5(4H)-one |
| 10 | 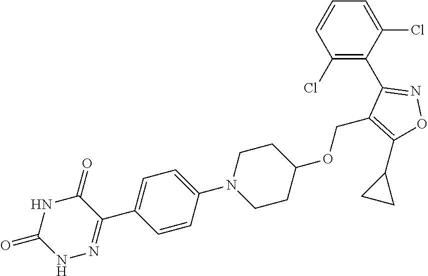 [SMILES: O=c1[nH]nc(-c2ccc(N3CCC(OCc4c(-c5c(Cl)cccc5Cl)noc4C4CC4)CC3)cc2)c(=O)[nH]1] | 6-(4-(4-((5-cyclopropyl-3-(2,6- dichlorophenyl)isoxazol-4- yl)methoxy)piperidin-1-yl)phenyl)- 1,2,4-triazine-3,5(2H,4H)-dione |
| 11 | 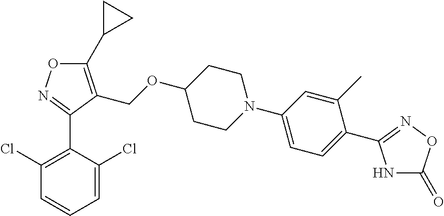 [SMILES: Cc1cc(N2CCC(OCc3c(-c4c(Cl)cccc4Cl)noc3C3CC3)CC2)ccc1-c1noc(=O)[nH]1] | 3-(4-(4-((5-cyclopropyl-3-(2,6- dichlorophenyl)isoxazol-4- yl)methoxy)piperidin-1-yl)-2- methylphenyl)-1,2,4-oxadiazol-5(4H)- one |
| 12 | 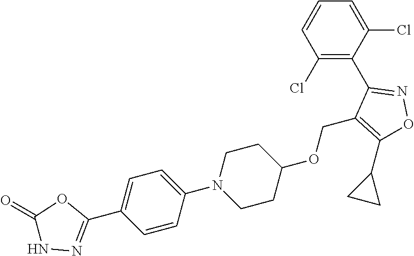 [SMILES: O=c1[nH]nc(-c2ccc(N3CCC(OCc4c(-c5c(Cl)cccc5Cl)noc4C4CC4)CC3)cc2)o1] | 5-(4-(4-((5-cyclopropyl-3-(2,6- dichlorophenyl)isoxazol-4- yl)methoxy)piperidin-1-yl)phenyl)- 1,3,4-oxadiazol-2(3H)-one |
| 13 | 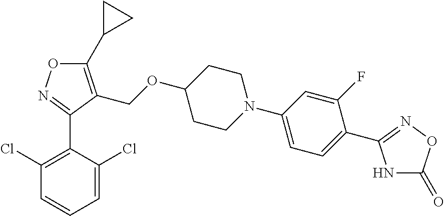 [SMILES: O=c1[nH]c(-c2ccc(N3CCC(OCc4c(-c5c(Cl)cccc5Cl)noc4C4CC4)CC3)cc2F)no1] | 3-(4-(4-((5-cyclopropyl-3-(2,6- dichlorophenyl)isoxazol-4- yl)methoxy)piperidin-1-yl)-2- fluorophenyl)-1,2,4-oxadiazol-5(4H)- one |
| 14 | 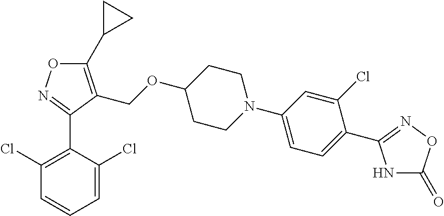 [SMILES: O=c1[nH]c(-c2ccc(N3CCC(OCc4c(-c5c(Cl)cccc5Cl)noc4C4CC4)CC3)cc2Cl)no1] | 3-(2-chloro-4-(4-((5-cyclopropyl-3- (2,6-dichlorophenyl)isoxazol-4- yl)methoxy)piperidin-1-yl)phenyl)- 1,2,4-oxadiazol-5(4H)-one |
| 15 | 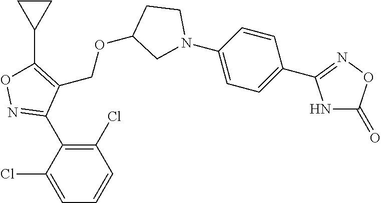 [SMILES: O=c1[nH]c(-c2ccc(N3CCC(OCc4c(-c5c(Cl)cccc5Cl)noc4C4CC4)C3)cc2)no1] | 3-(4-(3-((5-cyclopropyl-3-(2,6- dichlorophenyl)isoxazol-4- yl)methoxy)pyrrolidin-1-yl)phenyl)- 1,2,4-oxadiazol-5(4H)-one |
| 16 | 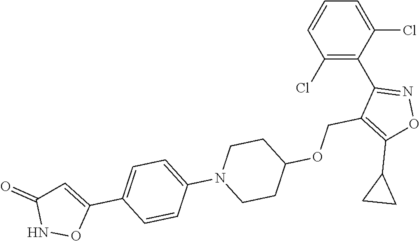 [SMILES: O=c1cc(-c2ccc(N3CCC(OCc4c(-c5c(Cl)cccc5Cl)noc4C4CC4)CC3)cc2)o[nH]1] | 5-(4-(4-((5-cyclopropyl-3-(2,6- dichlorophenyl)isoxazol-4- yl)methoxy)piperidin-1- yl)phenyl)isoxazol-3(2H)-one |
| 17 | 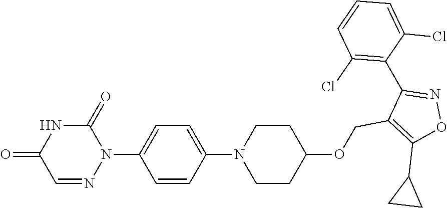 [SMILES: O=c1cnn(-c2ccc(N3CCC(OCc4c(-c5c(Cl)cccc5Cl)noc4C4CC4)CC3)cc2)c(=O)[nH]1] | 2-(4-(4-((5-cyclopropyl-3-(2,6- dichlorophenyl)isoxazol-4- yl)methoxy)piperidin-1-yl)phenyl)- 1,2,4-triazine-3,5(2H,4H)-dione |
| 18 | 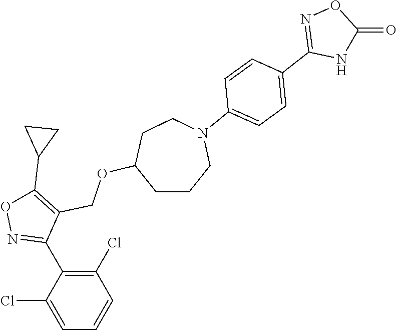 [SMILES: O=c1[nH]c(-c2ccc(N3CCCC(OCc4c(-c5c(Cl)cccc5Cl)noc4C4CC4)CC3)cc2)no1] | 3-(4-(4-((5-cyclopropyl-3-(2,6- dichlorophenyl)isoxazol-4- yl)methoxy)azepan-1-yl)phenyl)-1,2,4- oxadiazol-5(4H)-one |
| 19 | 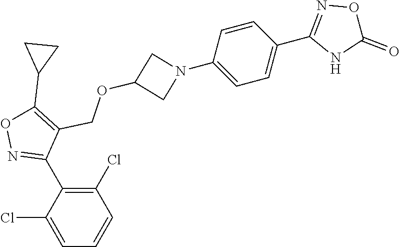 [SMILES: O=c1[nH]c(-c2ccc(N3CC(OCc4c(-c5c(Cl)cccc5Cl)noc4C4CC4)C3)cc2)no1] | 3-(4-(3-((5-cyclopropyl-3-(2,6- dichlorophenyl)isoxazol-4- yl)methoxy)azetidin-1-yl)phenyl)- 1,2,4-oxadiazol-5(4H)-one |
| 20 | 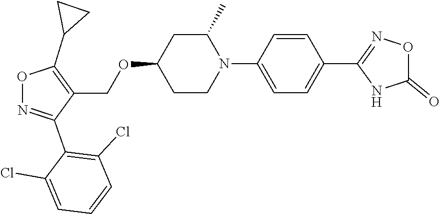 [SMILES: C[C@H]1C[C@H](OCc2c(-c3c(Cl)cccc3Cl)noc2C2CC2)CCN1c1ccc(-c2noc(=O)[nH]2)cc1] | 3-(4-((2S,4R)-4-((5-cyclopropyl-3-(2,6- dichlorophenyl)isoxazol-4- yl)methoxy)-2-methylpiperidin-1- yl)phenyl)-1,2,4-oxadiazol-5(4H)-one |
| 21 | 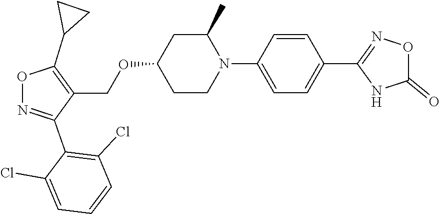 [SMILES: C[C@@H]1C[C@@H](OCc2c(-c3c(Cl)cccc3Cl)noc2C2CC2)CCN1c1ccc(-c2noc(=O)[nH]2)cc1] | 3-(4-((2R,4S)-4-((5-cyclopropyl-3-(2,6- dichlorophenyl)isoxazol-4- yl)methoxy)-2-methylpiperidin-1- yl)phenyl)-1,2,4-oxadiazol-5(4H)-one |
| 22 | 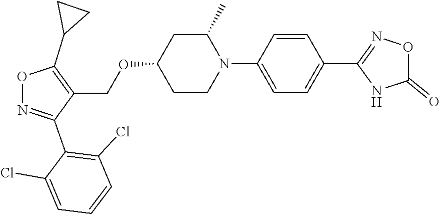 [SMILES: C[C@H]1C[C@@H](OCc2c(-c3c(Cl)cccc3Cl)noc2C2CC2)CCN1c1ccc(-c2noc(=O)[nH]2)cc1] | 3-(4-((2S,4S)-4-((5-cyclopropyl-3-(2,6- dichlorophenyl)isoxazol-4- yl)methoxy)-2-methylpiperidin-1- yl)phenyl)-1,2,4-oxadiazol-5(4H)-one |
| 23 | 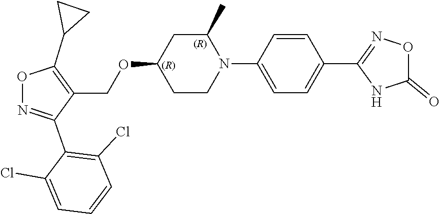 | 3-(4-((2R,4R)-4-((5-cyclopropyl-3- (2,6-dichlorophenyl)isoxazol-4- yl)methoxy)-2-methylpiperidin-1- yl)phenyl)-1,2,4-oxadiazol-5(4H)-one |
| 24 | 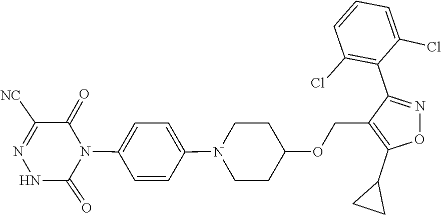 [SMILES: N#Cc1n[nH]c(=O)n(-c2ccc(N3CCC(OCc4c(-c5c(Cl)cccc5Cl)noc4C4CC4)CC3)cc2)c1=O] | 4-(4-(4-((5-cyclopropyl-3-(2,6- dichlorophenyl)isoxazol-4- yl)methoxy)piperidin-1-yl)phenyl)-3,5- dioxo-2,3,4,5-tetrahydro-1,2,4-triazine- 6-carbonitrile |
| 25 | 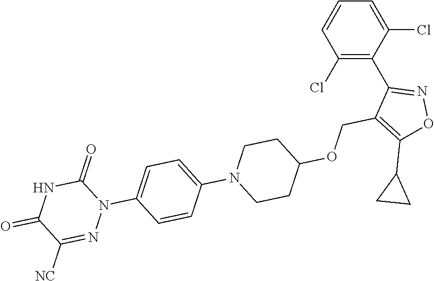 [SMILES: N#Cc1nn(-c2ccc(N3CCC(OCc4c(-c5c(Cl)cccc5Cl)noc4C4CC4)CC3)cc2)c(=O)[nH]c1=O] | 2-(4-(4-((5-cyclopropyl-3-(2,6- dichlorophenyl)isoxazol-4- yl)methoxy)piperidin-1-yl)phenyl)-3,5- dioxo-2,3,4,5-tetrahydro-1,2,4-triazine- 6-carbonitrile |
| 26 | 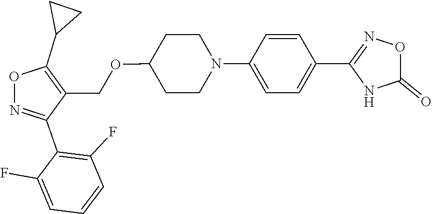 [SMILES: O=c1[nH]c(-c2ccc(N3CCC(OCc4c(-c5c(F)cccc5F)noc4C4CC4)CC3)cc2)no1] | 3-(4-(4-((5-cyclopropyl-3-(2,6- difluorophenyl)isoxazol-4- yl)methoxy)piperidin-1-yl)phenyl)- 1,2,4-oxadiazol-5(4H)-one |
| 27 | 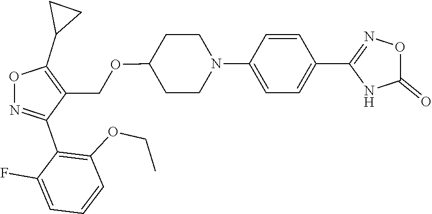 [SMILES: CCOc1cccc(F)c1-c1noc(C2CC2)c1COC1CCN(c2ccc(-c3noc(=O)[nH]3)cc2)CC1] | 3-(4-(4-((5-cyclopropyl-3-(2-ethoxy-6- fluorophenyl)isoxazol-4- yl)methoxy)piperidin-1-yl)phenyl)- 1,2,4-oxadiazol-5(4H)-one |
| 28 | 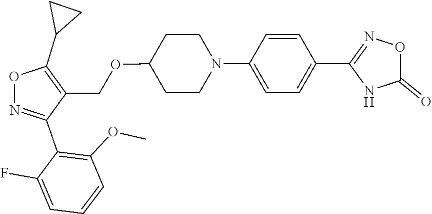 [SMILES: COc1cccc(F)c1-c1noc(C2CC2)c1COC1CCN(c2ccc(-c3noc(=O)[nH]3)cc2)CC1] | 3-(4-(4-((5-cyclopropyl-3-(2-fluoro-6- methoxyphenyl)isoxazol-4- yl)methoxy)piperidin-1-yl)phenyl)- 1,2,4-oxadiazol-5(4H)-one |
| 29 | 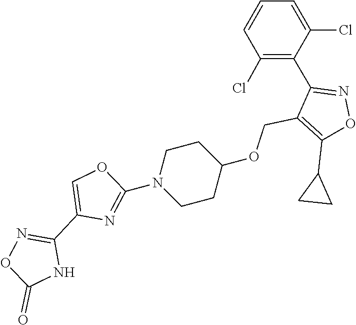 [SMILES: O=c1[nH]c(-c2coc(N3CCC(OCc4c(-c5c(Cl)cccc5Cl)noc4C4CC4)CC3)n2)no1] | 3-(2-(4-((5-cyclopropyl-3-(2,6- dichlorophenyl)isoxazol-4- yl)methoxy)piperidin-1-yl)oxazol-4- yl)-1,2,4-oxadiazol-5(4H)-one |
| 30 | 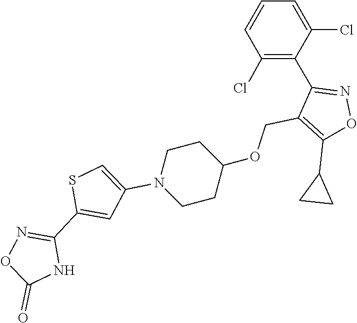 [SMILES: O=c1[nH]c(-c2cc(N3CCC(OCc4c(-c5c(Cl)cccc5Cl)noc4C4CC4)CC3)cs2)no1] | 3-(4-(4-((5-cyclopropyl-3-(2,6- dichlorophenyl)isoxazol-4- yl)methoxy)piperidin-1-yl)thiophen-2- yl)-1,2,4-oxadiazol-5(4H)-one |
| 31 | 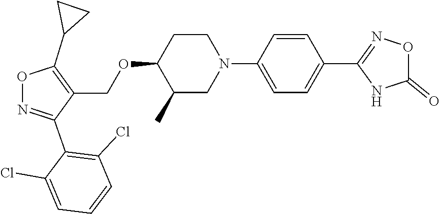 [SMILES: C[C@@H]1CN(c2ccc(-c3noc(=O)[nH]3)cc2)CC[C@@H]1OCc1c(-c2c(Cl)cccc2Cl)noc1C1CC1] | 3-(4-((3R,4S)-4-((5-cyclopropyl-3-(2,6- dichlorophenyl)isoxazol-4- yl)methoxy)-3-methylpiperidin-1- yl)phenyl)-1,2,4-oxadiazol-5(4H)-one |
| 32 | 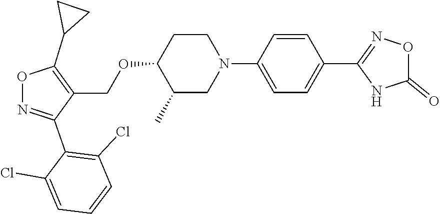 [SMILES: C[C@H]1CN(c2ccc(-c3noc(=O)[nH]3)cc2)CC[C@H]1OCc1c(-c2c(Cl)cccc2Cl)noc1C1CC1] | 3-(4-((3S,4R)-4-((5-cyclopropyl-3-(2,6- dichlorophenyl)isoxazol-4- yl)methoxy)-3-methylpiperidin-1- yl)phenyl)-1,2,4-oxadiazol-5(4H)-one |
| 33 | 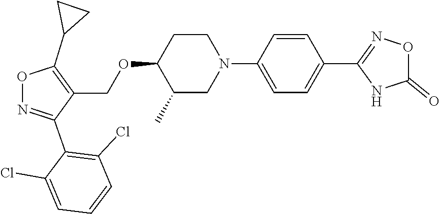 [SMILES: C[C@H]1CN(c2ccc(-c3noc(=O)[nH]3)cc2)CC[C@@H]1OCc1c(-c2c(Cl)cccc2Cl)noc1C1CC1] | 3-(4-((3S,4S)-4-((5-cyclopropyl-3-(2,6- dichlorophenyl)isoxazol-4- yl)methoxy)-3-methylpiperidin-1- yl)phenyl)-1,2,4-oxadiazol-5(4H)-one |
| 34 | 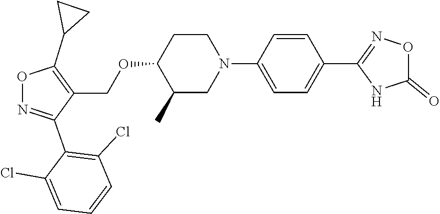 [SMILES: C[C@@H]1CN(c2ccc(-c3noc(=O)[nH]3)cc2)CC[C@H]1OCc1c(-c2c(Cl)cccc2Cl)noc1C1CC1] | 3-(4-((3R,4R)-4-((5-cyclopropyl-3- (2,6-dichlorophenyl)isoxazol-4- yl)methoxy)-3-methylpiperidin-1- yl)phenyl)-1,2,4-oxadiazol-5(4H)-one |
| 35 | 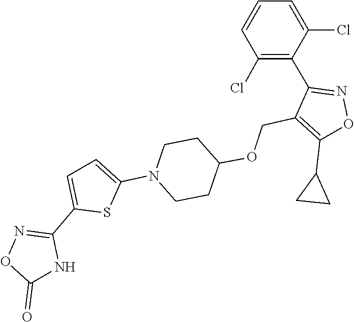 [SMILES: O=c1[nH]c(-c2ccc(N3CCC(OCc4c(-c5c(Cl)cccc5Cl)noc4C4CC4)CC3)s2)no1] | 3-(5-(4-((5-cyclopropyl-3-(2,6- dichlorophenyl)isoxazol-4- yl)methoxy)piperidin-1-yl)thiophen-2- yl)-1,2,4-oxadiazol-5(4H)-one |
| 36 | 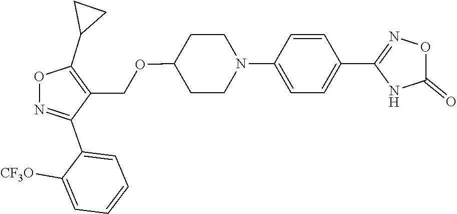 | 3-(4-(4-((5-cyclopropyl-3-(2- (trifluoromethoxy)phenyl)isoxazol-4- yl)methoxy)piperidin-1-yl)phenyl)- 1,2,4-oxadiazol-5(4H)-one |
| 37 | 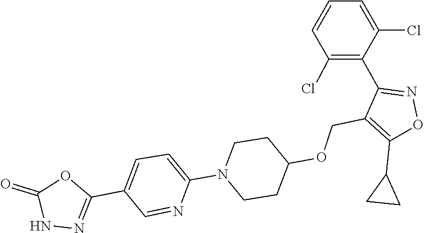 [SMILES: O=c1[nH]nc(-c2ccc(N3CCC(OCc4c(-c5c(Cl)cccc5Cl)noc4C4CC4)CC3)nc2)o1] | 5-(6-(4-((5-cyclopropyl-3-(2,6- dichlorophenyl)isoxazol-4- yl)methoxy)piperidin-1-yl)pyridin-3- yl)-1,3,4-oxadiazol-2(3H)-one |
| 38 | 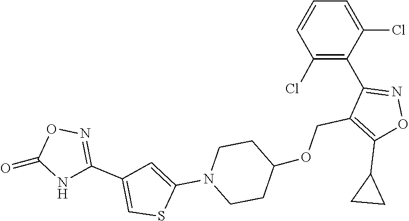 [SMILES: O=c1[nH]c(-c2csc(N3CCC(OCc4c(-c5c(Cl)cccc5Cl)noc4C4CC4)CC3)c2)no1] | 3-(5-(4-((5-cyclopropyl-3-(2,6- dichlorophenyl)isoxazol-4- yl)methoxy)piperidin-1-yl)thiophen-3- yl)-1,2,4-oxadiazol-5(4H)-one |
| 39 | 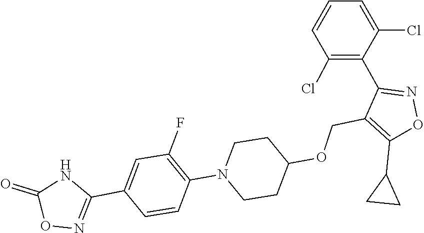 [SMILES: O=c1[nH]c(-c2ccc(N3CCC(OCc4c(-c5c(Cl)cccc5Cl)noc4C4CC4)CC3)c(F)c2)no1] | 3-(4-(4-((5-cyclopropyl-3-(2,6- dichlorophenyl)isoxazol-4- yl)methoxy)piperidin-1-yl)-3- fluorophenyl)-1,2,4-oxadiazol-5(4H)- one |
| 40 | 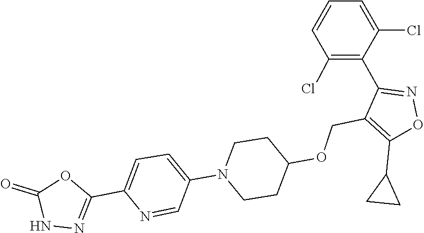 [SMILES: O=c1[nH]nc(-c2ccc(N3CCC(OCc4c(-c5c(Cl)cccc5Cl)noc4C4CC4)CC3)cn2)o1] | 5-(5-(4-((5-cyclopropyl-3-(2,6- dichlorophenyl)isoxazol-4- yl)methoxy)piperidin-1-yl)pyridin-2- yl)-1,3,4-oxadiazol-2(3H)-one |
| 41 | 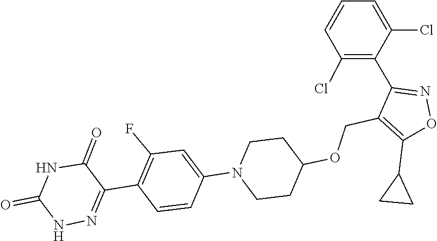 [SMILES: O=c1[nH]nc(-c2ccc(N3CCC(OCc4c(-c5c(Cl)cccc5Cl)noc4C4CC4)CC3)cc2F)c(=O)[nH]1] | 6-(4-(4-((5-cyclopropyl-3-(2,6- dichlorophenyl)isoxazol-4- yl)methoxy)piperidin-1-yl)-2- fluorophenyl)-1,2,4-triazine- 3,5(2H,4H)-dione |
| 42 | 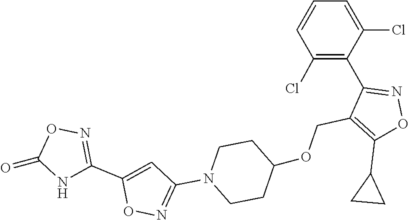 [SMILES: O=c1[nH]c(-c2cc(N3CCC(OCc4c(-c5c(Cl)cccc5Cl)noc4C4CC4)CC3)no2)no1] | 3-(3-(4-((5-cyclopropyl-3-(2,6- dichlorophenyl)isoxazol-4- yl)methoxy)piperidin-1-yl)isoxazol-5- yl)-1,2,4-oxadiazol-5(4H)-one |
| 43 | 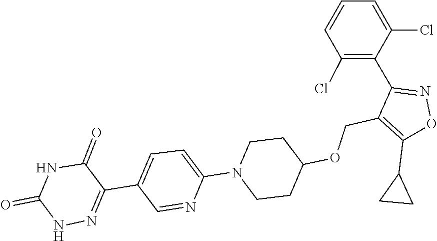 [SMILES: O=c1[nH]nc(-c2ccc(N3CCC(OCc4c(-c5c(Cl)cccc5Cl)noc4C4CC4)CC3)nc2)c(=O)[nH]1] | 6-(6-(4-((5-cyclopropyl-3-(2,6- dichlorophenyl)isoxazol-4- yl)methoxy)piperidin-1-yl)pyridin-3- yl)-1,2,4-triazine-3,5(2H,4H)-dione |
| 44 | 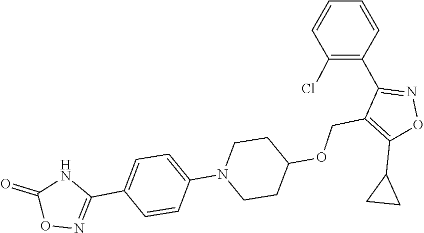 [SMILES: O=c1[nH]c(-c2ccc(N3CCC(OCc4c(-c5ccccc5Cl)noc4C4CC4)CC3)cc2)no1] | 3-(4-(4-((3-(2-chlorophenyl)-5- cyclopropylisoxazol-4- yl)methoxy)piperidin-1-yl)phenyl)- 1,2,4-oxadiazol-5(4H)-one |
| 45 | 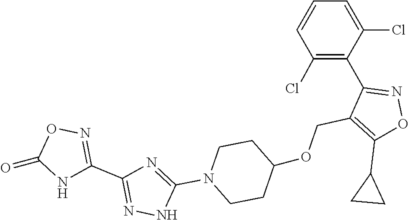 [SMILES: O=c1[nH]c(-c2n[nH]c(N3CCC(OCc4c(-c5c(Cl)cccc5Cl)noc4C4CC4)CC3)n2)no1] | 3-(5-(4-((5-cyclopropyl-3-(2,6- dichlorophenyl)isoxazol-4- yl)methoxy)piperidin-1-yl)-1H-1,2,4- triazol-3-yl)-1,2,4-oxadiazol-5(4H)-one |
| 46 | 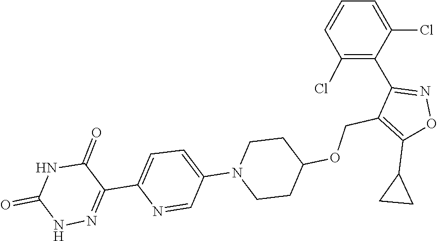 [SMILES: O=c1[nH]nc(-c2ccc(N3CCC(OCc4c(-c5c(Cl)cccc5Cl)noc4C4CC4)CC3)cn2)c(=O)[nH]1] | 6-(5-(4-((5-cyclopropyl-3-(2,6- dichlorophenyl)isoxazol-4- yl)methoxy)piperidin-1-yl)pyridin-2- yl)-1,2,4-triazine-3,5(2H,4H)-dione |
| 47 | 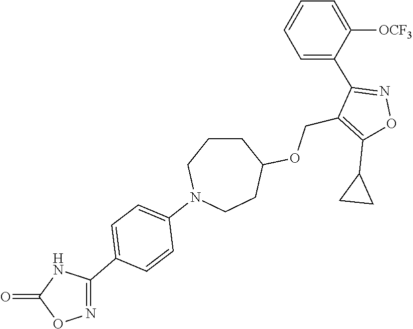 [SMILES: O=c1[nH]c(-c2ccc(N3CCCC(OCc4c(-c5ccccc5OC(F)(F)F)noc4C4CC4)CC3)cc2)no1] | 3-(4-(4-((5-cyclopropyl-3-(2- (trifluoromethoxy)phenyl)isoxazol-4- yl)methoxy)azepan-1-yl)phenyl)-1,2,4- oxadiazol-5(4H)-one |
| 48 | 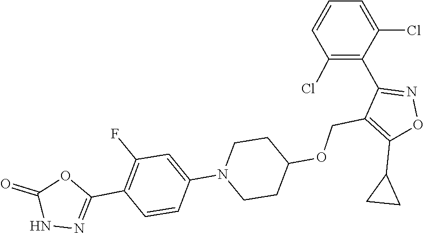 [SMILES: O=c1[nH]nc(-c2ccc(N3CCC(OCc4c(-c5c(Cl)cccc5Cl)noc4C4CC4)CC3)cc2F)o1] | 5-(4-(4-((5-cyclopropyl-3-(2,6- dichlorophenyl)isoxazol-4- yl)methoxy)piperidin-1-yl)-2- fluorophenyl)-1,3,4-oxadiazol-2(3H)- one |
| 49 | 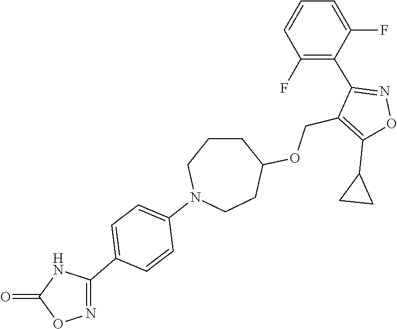 [SMILES: O=c1[nH]c(-c2ccc(N3CCCC(OCc4c(-c5c(F)cccc5F)noc4C4CC4)CC3)cc2)no1] | 3-(4-(4-((5-cyclopropyl-3-(2,6- difluorophenyl)isoxazol-4- yl)methoxy)azepan-1-yl)phenyl)-1,2,4- oxadiazol-5(4H)-one |
| 50 | 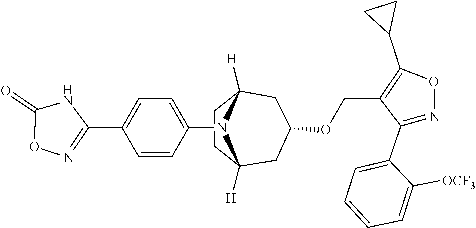 [SMILES: O=c1[nH]c(-c2ccc(N3[C@@H]4CC[C@H]3C[C@@H](OCc3c(-c5ccccc5OC(F)(F)F)noc3C3CC3)C4)cc2)no1] | 3-(4-((1R,3r,5S)-3-((5-cyclopropyl-3- (2-(trifluoromethoxy)phenyl)isoxazol- 4-yl)methoxy)-8- azabicyclo[3.2.1]octan-8-yl)phenyl)- 1,2,4-oxadiazol-5(4H)-one |
| 51 | 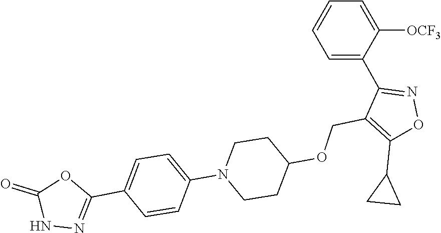 [SMILES: O=c1[nH]nc(-c2ccc(N3CCC(OCc4c(-c5ccccc5OC(F)(F)F)noc4C4CC4)CC3)cc2)o1] | 5-(4-(4-((5-cyclopropyl-3-(2- (trifluoromethoxy)phenyl)isoxazol-4- yl)methoxy)piperidin-1-yl)phenyl)- 1,3,4-oxadiazol-2(3H)-one |
| 52 | 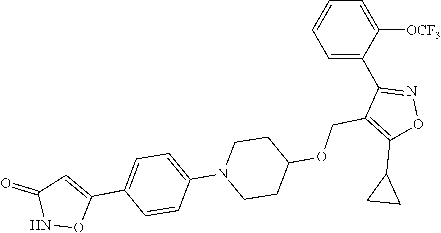 [SMILES: O=c1cc(-c2ccc(N3CCC(OCc4c(-c5ccccc5OC(F)(F)F)noc4C4CC4)CC3)cc2)o[nH]1] | 5-(4-(4-((5-cyclopropyl-3-(2- (trifluoromethoxy)phenyl)isoxazol-4- yl)methoxy)piperidin-1- yl)phenyl)isoxazol-3(2H)-one |
| 53 | 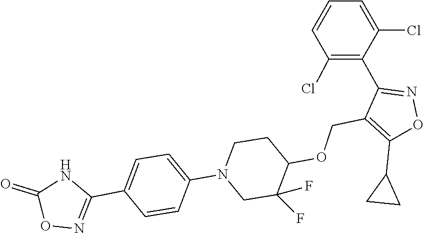 [SMILES: O=c1[nH]c(-c2ccc(N3CCC(OCc4c(-c5c(Cl)cccc5Cl)noc4C4CC4)C(F)(F)C3)cc2)no1] | 3-(4-(4-((5-cyclopropyl-3-(2,6- dichlorophenyl)isoxazol-4- yl)methoxy)-3,3-difluoropiperidin-1- yl)phenyl)-1,2,4-oxadiazol-5(4H)-one |
| 54 | 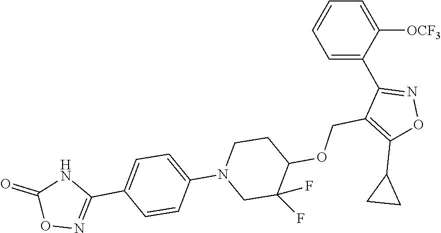 [SMILES: O=c1[nH]c(-c2ccc(N3CCC(OCc4c(-c5ccccc5OC(F)(F)F)noc4C4CC4)C(F)(F)C3)cc2)no1] | 3-(4-(4-((5-cyclopropyl-3-(2- (trifluoromethoxy)phenyl)isoxazol-4- yl)methoxy)-3,3-difluoropiperidin-1- yl)phenyl)-1,2,4-oxadiazol-5(4H)-one |
| 55 | 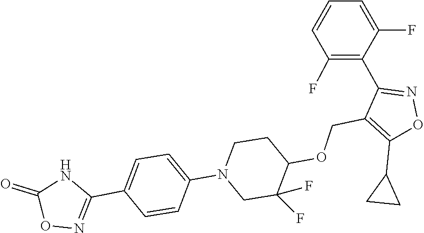 [SMILES: O=c1[nH]c(-c2ccc(N3CCC(OCc4c(-c5c(F)cccc5F)noc4C4CC4)C(F)(F)C3)cc2)no1] | 3-(4-(4-((5-cyclopropyl-3-(2,6- difluorophenyl)isoxazol-4- yl)methoxy)-3,3-difluoropiperidin-1- yl)phenyl)-1,2,4-oxadiazol-5(4H)-one |
| 56 | 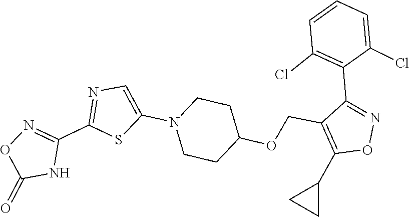 [SMILES: O=c1[nH]c(-c2ncc(N3CCC(OCc4c(-c5c(Cl)cccc5Cl)noc4C4CC4)CC3)s2)no1] | 3-(5-(4-((5-cyclopropyl-3-(2,6- dichlorophenyl)isoxazol-4- yl)methoxy)piperidin-1-yl)thiazol-2- yl)-1,2,4-oxadiazol-5(4H)-one |
| 57 | 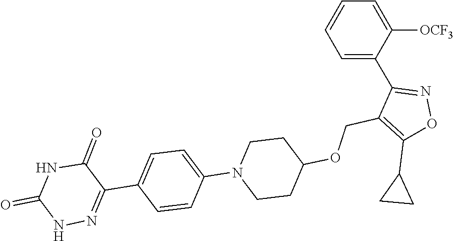 [SMILES: O=c1[nH]nc(-c2ccc(N3CCC(OCc4c(-c5ccccc5OC(F)(F)F)noc4C4CC4)CC3)cc2)c(=O)[nH]1] | 6-(4-(4-((5-cyclopropyl-3-(2- (trifluoromethoxy)phenyl)isoxazol-4- yl)methoxy)piperidin-1-yl)phenyl)- 1,2,4-triazine-3,5(2H,4H)-dione |
| 58 | 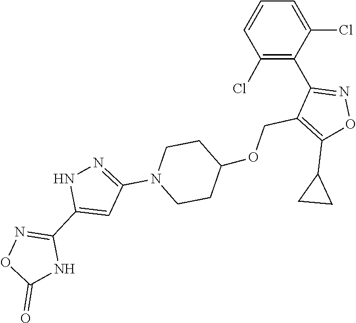 [SMILES: O=c1[nH]c(-c2cc(N3CCC(OCc4c(-c5c(Cl)cccc5Cl)noc4C4CC4)CC3)n[nH]2)no1] | 3-(3-(4-((5-cyclopropyl-3-(2,6- dichlorophenyl)isoxazol-4- yl)methoxy)piperidin-1-yl)-1H- pyrazol-5-yl)-1,2,4-oxadiazol-5(4H)- one |
| 59 | 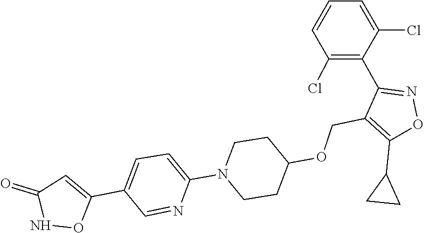 [SMILES: O=c1cc(-c2ccc(N3CCC(OCc4c(-c5c(Cl)cccc5Cl)noc4C4CC4)CC3)nc2)o[nH]1] | 5-(6-(4-((5-cyclopropyl-3-(2,6- dichlorophenyl)isoxazol-4- yl)methoxy)piperidin-1-yl)pyridin-3- yl)isoxazol-3(2H)-one |
| 60 | 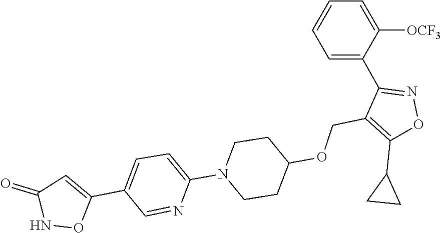 [SMILES: O=c1cc(-c2ccc(N3CCC(OCc4c(-c5ccccc5OC(F)(F)F)noc4C4CC4)CC3)nc2)o[nH]1] | 5-(6-(4-((5-cyclopropyl-3-(2- (trifluoromethoxy)phenyl)isoxazol-4- yl)methoxy)piperidin-1-yl)pyridin-3- yl)isoxazol-3(2H)-one |
| 61 | 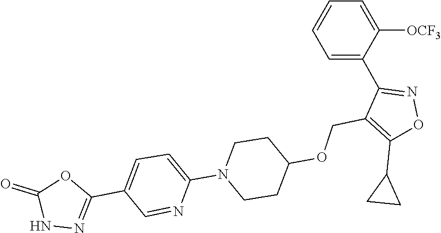 [SMILES: O=c1[nH]nc(-c2ccc(N3CCC(OCc4c(-c5ccccc5OC(F)(F)F)noc4C4CC4)CC3)nc2)o1] | 5-(6-(4-((5-cyclopropyl-3-(2- (trifluoromethoxy)phenyl)isoxazol-4- yl)methoxy)piperidin-1-yl)pyridin-3- yl)-1,3,4-oxadiazol-2(3H)-one |
| 62 | 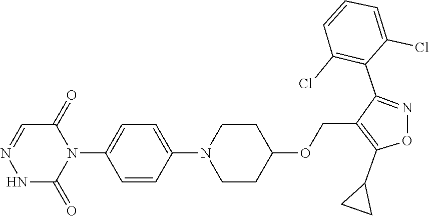 [SMILES: O=c1cn[nH]c(=O)n1-c1ccc(N2CCC(OCc3c(-c4c(Cl)cccc4Cl)noc3C3CC3)CC2)cc1] | 4-(4-(4-((5-cyclopropyl-3-(2,6- dichlorophenyl)isoxazol-4- yl)methoxy)piperidin-1-yl)phenyl)- 1,2,4-triazine-3,5(2H,4H)-dione |
| 63 | 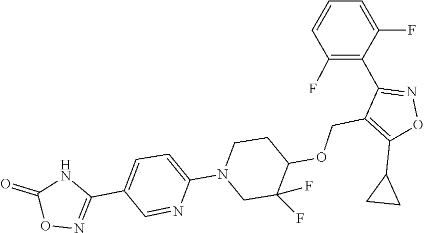 [SMILES: O=c1[nH]c(-c2ccc(N3CCC(OCc4c(-c5c(F)cccc5F)noc4C4CC4)C(F)(F)C3)nc2)no1] | 3-(6-(4-((5-cyclopropyl-3-(2,6- difluorophenyl)isoxazol-4- yl)methoxy)-3,3-difluoropiperidin-1- yl)pyridin-3-yl)-1,2,4-oxadiazol-5(4H)- one |
| 64 | 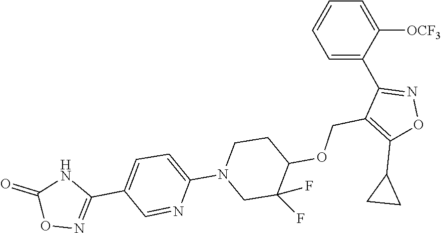 [SMILES: O=c1[nH]c(-c2ccc(N3CCC(OCc4c(-c5ccccc5OC(F)(F)F)noc4C4CC4)C(F)(F)C3)nc2)no1] | 3-(6-(4-((5-cyclopropyl-3-(2- (trifluoromethoxy)phenyl)isoxazol-4- yl)methoxy)-3,3-difluoropiperidin-1- yl)pyridin-3-yl)-1,2,4-oxadiazol-5(4H)- one |
| 65 | 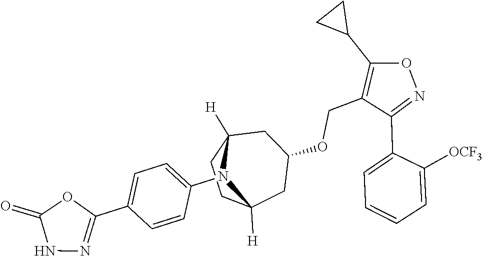 [SMILES: O=c1[nH]nc(-c2ccc(N3[C@@H]4CC[C@H]3C[C@@H](OCc3c(-c5ccccc5OC(F)(F)F)noc3C3CC3)C4)cc2)o1] | 5-(4-((1R,3r,5S)-3-((5-cyclopropyl-3- (2-(trifluoromethoxy)phenyl)isoxazol- 4-yl)methoxy)-8- azabicyclo[3.2.1]octan-8-yl)phenyl)- 1,3,4-oxadiazol-2(3H)-one |
| 66 | 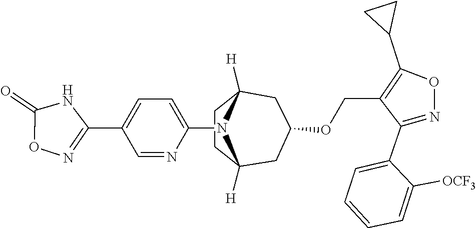 [SMILES: O=c1[nH]c(-c2ccc(N3[C@@H]4CC[C@H]3C[C@@H](OCc3c(-c5ccccc5OC(F)(F)F)noc3C3CC3)C4)nc2)no1] | 3-(6-((1R,3r,5S)-3-((5-cyclopropyl-3- (2-(trifluoromethoxy)phenyl)isoxazol- 4-yl)methoxy)-8- azabicyclo[3.2.1]octan-8-yl)pyridin-3- yl)-1,2,4-oxadiazol-5(4H)-one |
| 67 | 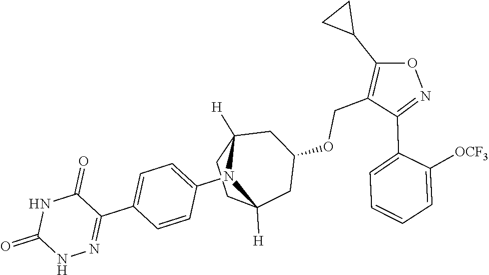 [SMILES: O=c1[nH]nc(-c2ccc(N3[C@@H]4CC[C@H]3C[C@@H](OCc3c(-c5ccccc5OC(F)(F)F)noc3C3CC3)C4)cc2)c(=O)[nH]1] | 6-(4-((1R,3r,5S)-3-((5-cyclopropyl-3- (2-(trifluoromethoxy)phenyl)isoxazol- 4-yl)methoxy)-8- azabicyclo[3.2.1]octan-8-yl)phenyl)- 1,2,4-triazine-3,5(2H,4H)-dione |
| 68 | 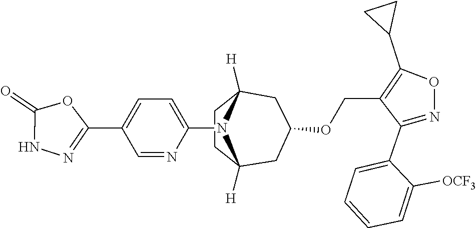 [SMILES: O=c1[nH]nc(-c2ccc(N3[C@@H]4CC[C@H]3C[C@@H](OCc3c(-c5ccccc5OC(F)(F)F)noc3C3CC3)C4)nc2)o1] | 5-(6-((1R,3r,5S)-3-((5-cyclopropyl-3- (2-(trifluoromethoxy)phenyl)isoxazol- 4-yl)methoxy)-8- azabicyclo[3.2.1]octan-8-yl)pyridin-3- yl)-1,3,4-oxadiazol-2(3H)-one |
| 69 | 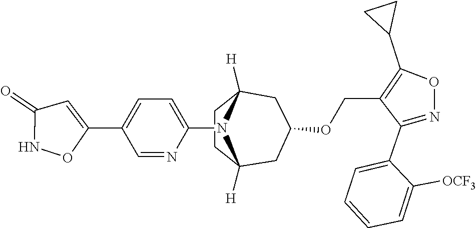 [SMILES: O=c1cc(-c2ccc(N3[C@@H]4CC[C@H]3C[C@@H](OCc3c(-c5ccccc5OC(F)(F)F)noc3C3CC3)C4)nc2)o[nH]1] | 5-(6-((1R,3r,5S)-3-((5-cyclopropyl-3- (2-(trifluoromethoxy)phenyl)isoxazol- 4-yl)methoxy)-8- azabicyclo[3.2.1]octan-8-yl)pyridin-3- yl)isoxazol-3(2H)-one |
| 70 | 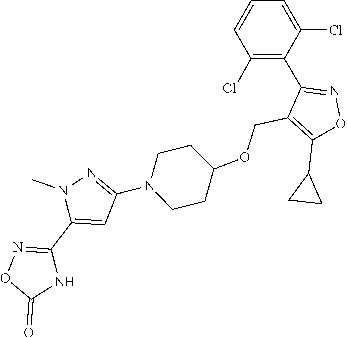 [SMILES: Cn1nc(N2CCC(OCc3c(-c4c(Cl)cccc4Cl)noc3C3CC3)CC2)cc1-c1noc(=O)[nH]1] | 3-(3-(4-((5-cyclopropyl-3-(2,6- dichlorophenyl)isoxazol-4- yl)methoxy)piperidin-1-yl)-1-methyl- 1H-pyrazol-5-yl)-1,2,4-oxadiazol- 5(4H)-one |
| 71 | 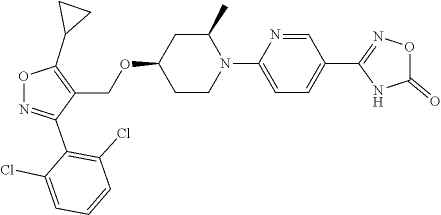 [SMILES: C[C@@H]1C[C@H](OCc2c(-c3c(Cl)cccc3Cl)noc2C2CC2)CCN1c1ccc(-c2noc(=O)[nH]2)cn1] | 3-(6-((2R,4R)-4-((5-cyclopropyl-3- (2,6-dichlorophenyl)isoxazol-4- yl)methoxy)-2-methylpiperidin-1- yl)pyridin-3-yl)-1,2,4-oxadiazol-5(4H)- one |
| 72 | 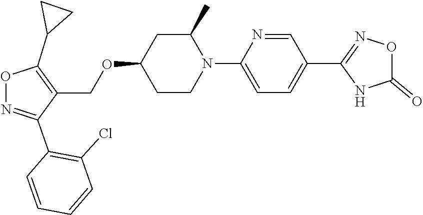 [SMILES: C[C@@H]1C[C@H](OCc2c(-c3ccccc3Cl)noc2C2CC2)CCN1c1ccc(-c2noc(=O)[nH]2)cn1] | 3-(6-((2R,4R)-4-((3-(2-chlorophenyl)- 5-cyclopropylisoxazol-4-yl)methoxy)- 2-methylpiperidin-1-yl)pyridin-3-yl)- 1,2,4-oxadiazol-5(4H)-one |
| 73 | 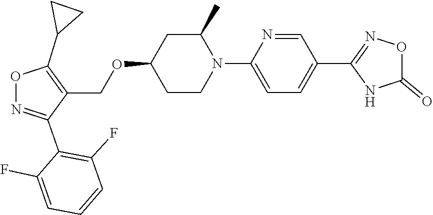 [SMILES: C[C@@H]1C[C@H](OCc2c(-c3c(F)cccc3F)noc2C2CC2)CCN1c1ccc(-c2noc(=O)[nH]2)cn1] | 3-(6-((2R,4R)-4-((5-cyclopropyl-3- (2,6-difluorophenyl)isoxazol-4- yl)methoxy)-2-methylpiperidin-1- yl)pyridin-3-yl)-1,2,4-oxadiazol-5(4H)- one |
| 74 | 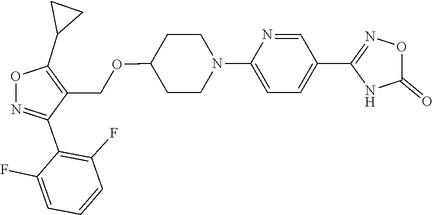 [SMILES: O=c1[nH]c(-c2ccc(N3CCC(OCc4c(-c5c(F)cccc5F)noc4C4CC4)CC3)nc2)no1] | 3-(6-(4-((5-cyclopropyl-3-(2,6- difluorophenyl)isoxazol-4- yl)methoxy)piperidin-1-yl)pyridin-3- yl)-1,2,4-oxadiazol-5(4H)-one |
| 75 | 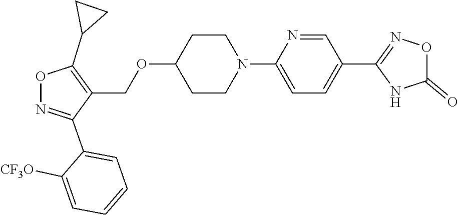 | 3-(6-(4-((5-cyclopropyl-3-(2- (trifluoromethoxy)phenyl)isoxazol-4- yl)methoxy)piperidin-1-yl)pyridin-3- yl)-1,2,4-oxadiazol-5(4H)-one |
| 76 | 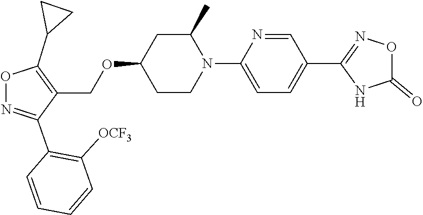 [SMILES: C[C@@H]1C[C@H](OCc2c(-c3ccccc3OC(F)(F)F)noc2C2CC2)CCN1c1ccc(-c2noc(=O)[nH]2)cn1] | 3-(6-((2R,4R)-4-((5-cyclopropyl-3-(2- (trifluoromethoxy)phenyl)isoxazol-4- yl)methoxy)-2-methylpiperidin-1- yl)pyridin-3-yl)-1,2,4-oxadiazol-5(4H)- one |
| 77 | 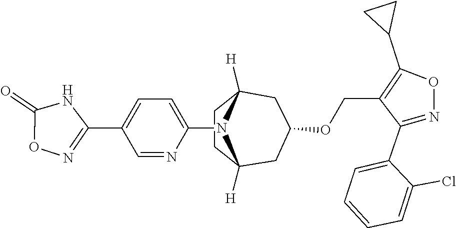 [SMILES: O=c1[nH]c(-c2ccc(N3[C@@H]4CC[C@H]3C[C@@H](OCc3c(-c5ccccc5Cl)noc3C3CC3)C4)nc2)no1] | 3-(6-((1R,3r,5S)-3-((3-(2- chlorophenyl)-5-cyclopropylisoxazol- 4-yl)methoxy)-8- azabicyclo[3.2.1]octan-8-yl)pyridin-3- yl)-1,2,4-oxadiazol-5(4H)-one |
| 78 | 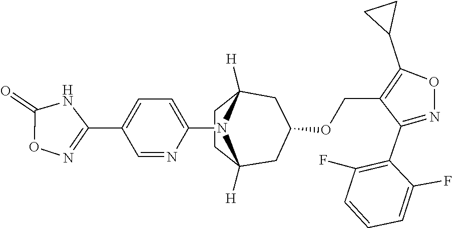 [SMILES: O=c1[nH]c(-c2ccc(N3[C@@H]4CC[C@H]3C[C@@H](OCc3c(-c5c(F)cccc5F)noc3C3CC3)C4)nc2)no1] | 3-(6-((1R,3r,5S)-3-((5-cyclopropyl-3- (2,6-difluorophenyl)isoxazol-4- yl)methoxy)-8-azabicyclo[3.2.1]octan- 8-yl)pyridin-3-yl)-1,2,4-oxadiazol- 5(4H)-one |
| 79 | 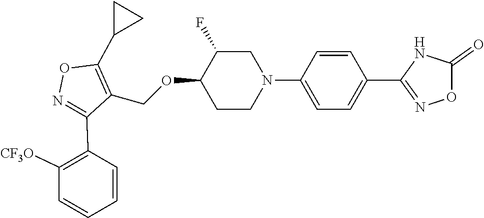 | 3-(4-((3R,4R)-4-((5-cyclopropyl-3-(2- (trifluoromethoxy)phenyl)isoxazol-4- yl)methoxy)-3-fluoropiperidin-1- yl)phenyl)-1,2,4-oxadiazol-5(4H)-one |
| 80 | 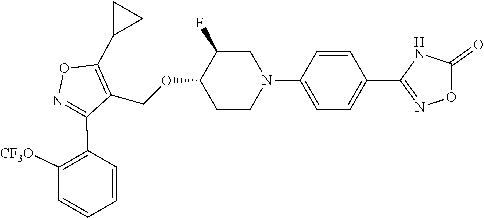 | 3-(4-((3S,4S)-4-((5-cyclopropyl-3-(2- (trifluoromethoxy)phenyl)isoxazol-4- yl)methoxy)-3-fluoropiperidin-1- yl)phenyl)-1,2,4-oxadiazol-5(4H)-one |
| 81 | 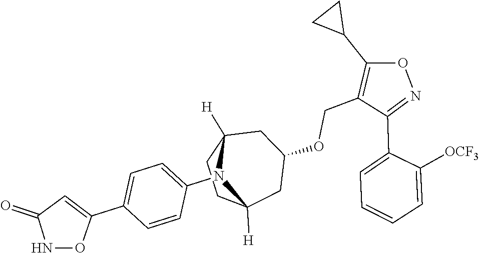 [SMILES: O=c1cc(-c2ccc(N3[C@@H]4CC[C@H]3C[C@@H](OCc3c(-c5ccccc5OC(F)(F)F)noc3C3CC3)C4)cc2)o[nH]1] | 5-(4-((1R,3r,5S)-3-((5-cyclopropyl-3- (2-(trifluoromethoxy)phenyl)isoxazol- 4-yl)methoxy)-8- azabicyclo[3.2.1]octan-8- yl)phenyl)isoxazol-3(2H)-one |
| 82 | 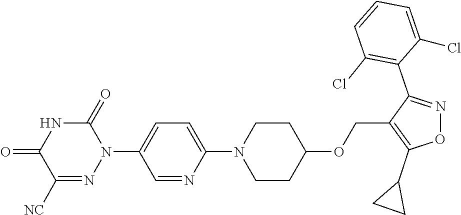 [SMILES: N#Cc1nn(-c2ccc(N3CCC(OCc4c(-c5c(Cl)cccc5Cl)noc4C4CC4)CC3)nc2)c(=O)[nH]c1=O] | 2-(6-(4-((5-cyclopropyl-3-(2,6- dichlorophenyl)isoxazol-4- yl)methoxy)piperidin-1-yl)pyridin-3- yl)-3,5-dioxo-2,3,4,5-tetrahydro-1,2,4- triazine-6-carbonitrile |
| 83 | 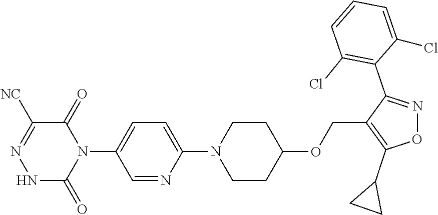 [SMILES: N#Cc1n[nH]c(=O)n(-c2ccc(N3CCC(OCc4c(-c5c(Cl)cccc5Cl)noc4C4CC4)CC3)nc2)c1=O] | 4-(6-(4-((5-cyclopropyl-3-(2,6- dichlorophenyl)isoxazol-4- yl)methoxy)piperidin-1-yl)pyridin-3- yl)-3,5-dioxo-2,3,4,5-tetrahydro-1,2,4- triazine-6-carbonitrile |
| 84 | 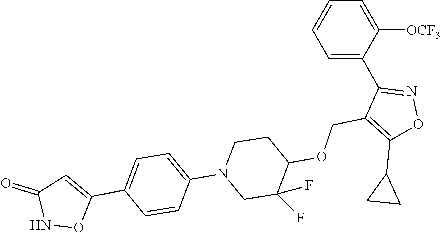 [SMILES: O=c1cc(-c2ccc(N3CCC(OCc4c(-c5ccccc5OC(F)(F)F)noc4C4CC4)C(F)(F)C3)cc2)o[nH]1] | 5-(4-(4-((5-cyclopropyl-3-(2- (trifluoromethoxy)phenyl)isoxazol-4- yl)methoxy)-3,3-difluoropiperidin-1- yl)phenyl)isoxazol-3(2H)-one |
| 85 | 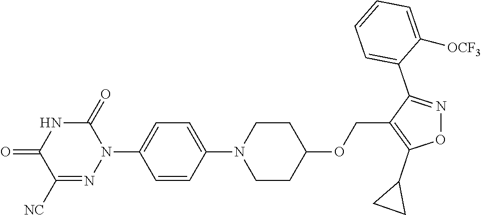 [SMILES: N#Cc1nn(-c2ccc(N3CCC(OCc4c(-c5ccccc5OC(F)(F)F)noc4C4CC4)CC3)cc2)c(=O)[nH]c1=O] | 2-(4-(4-((5-cyclopropyl-3-(2- (trifluoromethoxy)phenyl)isoxazol-4- yl)methoxy)piperidin-1-yl)phenyl)-3,5- dioxo-2,3,4,5-tetrahydro-1,2,4-triazine- 6-carbonitrile |
| 86 | 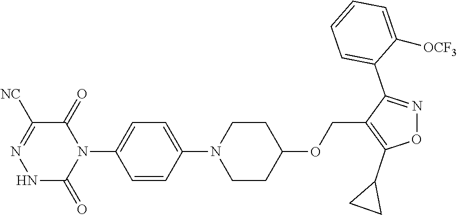 [SMILES: N#Cc1n[nH]c(=O)n(-c2ccc(N3CCC(OCc4c(-c5ccccc5OC(F)(F)F)noc4C4CC4)CC3)cc2)c1=O] | 4-(4-(4-((5-cyclopropyl-3-(2- (trifluoromethoxy)phenyl)isoxazol-4- yl)methoxy)piperidin-1-yl)phenyl)-3,5- dioxo-2,3,4,5-tetrahydro-1,2,4-triazine- 6-carbonitrile |
| 87 | 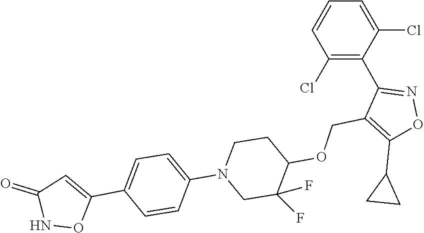 [SMILES: O=c1cc(-c2ccc(N3CCC(OCc4c(-c5c(Cl)cccc5Cl)noc4C4CC4)C(F)(F)C3)cc2)o[nH]1] | 5-(4-(4-((5-cyclopropyl-3-(2,6- dichlorophenyl)isoxazol-4- yl)methoxy)-3,3-difluoropiperidin-1- yl)phenyl)isoxazol-3(2H)-one |
| 88 | 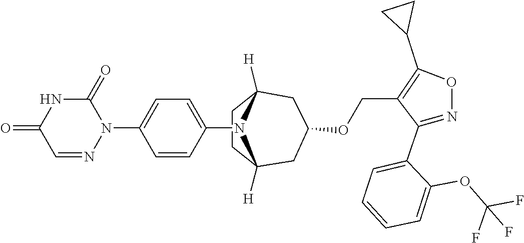 [SMILES: O=c1cnn(-c2ccc(N3[C@@H]4CC[C@H]3C[C@@H](OCc3c(-c5ccccc5OC(F)(F)F)noc3C3CC3)C4)cc2)c(=O)[nH]1] | 2-(4-((1R,3r,5S)-3-((5-cyclopropyl-3- (2-(trifluoromethoxy)phenyl)isoxazol- 4-yl)methoxy)-8- azabicyclo[3.2.1]octan-8-yl)phenyl)- 1,2,4-triazine-3,5(2H,4H)-dione |
| 89 | 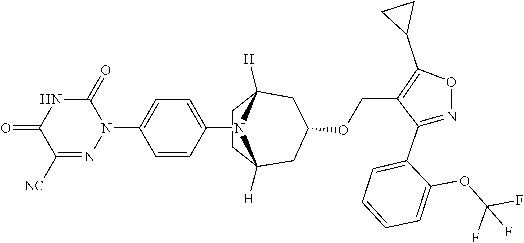 [SMILES: N#Cc1nn(-c2ccc(N3[C@@H]4CC[C@H]3C[C@@H](OCc3c(-c5ccccc5OC(F)(F)F)noc3C3CC3)C4)cc2)c(=O)[nH]c1=O] | 2-(4-((1R,3r,5S)-3-((5-cyclopropyl-3- (2-(trifluoromethoxy)phenyl)isoxazol- 4-yl)methoxy)-8- azabicyclo[3.2.1]octan-8-yl)phenyl)- 3,5-dioxo-2,3,4,5-tetrahydro-1,2,4- triazine-6-carbonitrile |
| 90 | 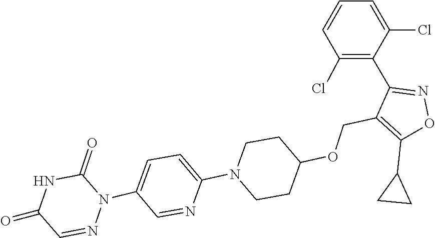 [SMILES: O=c1cnn(-c2ccc(N3CCC(OCc4c(-c5c(Cl)cccc5Cl)noc4C4CC4)CC3)nc2)c(=O)[nH]1] | 2-(6-(4-((5-cyclopropyl-3-(2,6- dichlorophenyl)isoxazol-4- yl)methoxy)piperidin-1-yl)pyridin-3- yl)-1,2,4-triazine-3,5(2H,4H)-dione |

In another aspect, provided is a method of making a compound of Formula (I), or a stereoisomer, tautomer, or a pharmaceutically acceptable salt of any of the foregoing. Compounds described herein may be prepared according to general schemes, as exemplified by the general procedures and examples. Minor variations in temperatures, concentrations, reaction times, and other parameters can be made when following the general procedures, which do not substantially affect the results of the procedures.

Also provided are compound intermediates useful in synthesis of a compound of Formula (I), or a stereoisomer, tautomer, or a pharmaceutically acceptable salt of any of the foregoing. Synthesis of representative compounds and intermediates are shown in the examples below.

The compounds depicted herein may be present as salts even if salts are not depicted and it is understood that the present disclosure embraces all salts and solvates of the compounds depicted here, as well as the non-salt and non-solvate form of the compound, as is well understood by the skilled artisan. In some embodiments, the salts of the compounds provided herein are pharmaceutically acceptable salts. Where one or more tertiary amine moiety is present in the compound, the N-oxides are also provided and described.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described. Compounds of any formula given herein may have asymmetric centers and therefore exist in different enantiomeric or diastereomeric forms. All optical isomers and stereoisomers of the compounds of the general formula, and mixtures thereof in any ratio, are considered within the scope of the formula. Thus, any formula given herein is intended to represent a racemate, one or more enantiomeric forms, one or more diastereomeric forms, one or more atropisomeric forms, and mixtures thereof in any ratio, unless a specific stereochemistry is otherwise indicated. Where a compound of Table 1 is depicted with a particular stereochemical configuration, also provided herein is any alternative stereochemical configuration of the compound, as well as a mixture of stereoisomers of the compound in any ratio. For example, where a compound of Table 1 has a stereocenter that is in an “S” stereochemical configuration, also provided herein is the enantiomer of the compound wherein that stereocenter is in an “R” stereochemical configuration. Likewise, when a compound of Table 1 has a stereocenter that is in an “R” configuration, also provided herein is enantiomer of the compound in an “S” stereochemical configuration. Also provided are mixtures of the compound with both the “S” and the “R” stereochemical configuration.

The disclosure also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds of the disclosure include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution study. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds of the present disclosure can generally be prepared by standard methods and techniques known to those skilled in the art or by procedures similar to those described in the accompanying Examples substituting appropriate isotopically-labeled reagents in place of the corresponding non-labeled reagent.

The disclosure also includes any or all metabolites of any of the compounds described. The metabolites may include any chemical species generated by a biotransformation of any of the compounds described, such as intermediates and products of metabolism of the compound, such as would be generated in vivo following administration to a human.

### Pharmaceutically Acceptable Compositions and Formulations

Pharmaceutically acceptable compositions or simply “pharmaceutical compositions” of any of the compounds detailed herein are embraced by this disclosure. Thus, the disclosure includes pharmaceutical compositions comprising a compound of Formula (I) (including compounds of Formulae (II)-(IV)), or a stereoisomer, tautomer, or a pharmaceutically acceptable salt of any of the foregoing, and a pharmaceutically acceptable carrier or excipient.

In some embodiments, the pharmaceutically acceptable salt is an acid addition salt, such as a salt formed with an inorganic or organic acid. Pharmaceutical compositions according to the disclosure may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. In one variation, “substantially pure” intends a composition that contains no more than 35% impurity, wherein the impurity denotes a compound other than the compound comprising the majority of the composition or a salt thereof. For example, a composition of a substantially pure compound intends a composition that contains no more than 35% impurity, wherein the impurity denotes a compound other than the compound or a salt thereof. In one variation, a composition of substantially pure compound or a salt thereof is provided wherein the composition contains no more than 25% impurity. In another variation, a composition of substantially pure compound or a salt thereof is provided wherein the composition contains or no more than 20% impurity. In still another variation, a composition of substantially pure compound or a salt thereof is provided wherein the composition contains or no more than 10% impurity. In a further variation, a composition of substantially pure compound or a salt thereof is provided wherein the composition contains or no more than 5% impurity. In another variation, a composition of substantially pure compound or a salt thereof is provided wherein the composition contains or no more than 3% impurity. In still another variation, a composition of substantially pure compound or a salt thereof is provided wherein the composition contains or no more than 1% impurity. In a further variation, a composition of substantially pure compound or a salt thereof is provided wherein the composition contains or no more than 0.5% impurity. In yet other variations, a composition of substantially pure compound means that the composition contains no more than 15% or preferably no more than 10% or more preferably no more than 5% or even more preferably no more than 3% and most preferably no more than 1% impurity, which impurity may be the compound in a different stereochemical form.

In one variation, the compounds herein are synthetic compounds prepared for administration to an individual such as a human. In another variation, compositions are provided containing a compound in substantially pure form. In another variation, the disclosure embraces pharmaceutical compositions comprising a compound detailed herein and a pharmaceutically acceptable carrier or excipient. In another variation, methods of administering a compound are provided. The purified forms, pharmaceutical compositions and methods of administering the compounds are suitable for any compound or form thereof detailed herein.

The compounds may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

Compounds described herein can be used in the preparation of a formulation, such as a pharmaceutical formulation, by combining the compounds as active ingredients with a pharmaceutically acceptable carrier, such as those mentioned above. Depending on the therapeutic form of the system (e.g., transdermal patch vs. oral tablet), the carrier may be in various forms. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants. Formulations comprising the compound may also contain other substances which have valuable therapeutic properties. Pharmaceutical formulations may be prepared by known pharmaceutical methods. Suitable formulations can be found, e.g., in Remington: The Science and Practice of Pharmacy, Lippincott Williams & Wilkins, 21^{st} ed. (2005), which is incorporated herein by reference.

Compounds as described herein may be administered to individuals (e.g., a human) in a form of generally accepted oral compositions, such as tablets, coated tablets, and gel capsules in a hard or in soft shell, emulsions or suspensions. Examples of carriers, which may be used for the preparation of such compositions, are lactose, corn starch or its derivatives, talc, stearate or its salts, etc. Acceptable carriers for gel capsules with soft shell are, for instance, plant oils, wax, fats, semisolid and liquid polyols, and so on. In addition, pharmaceutical formulations may contain preservatives, solubilizers, stabilizers, re-wetting agents, emulgators, sweeteners, dyes, adjusters, and salts for the adjustment of osmotic pressure, buffers, coating agents or antioxidants.

### Methods of Use and Uses

Compounds and compositions described herein may in some aspects be used in treatment of diseases and/or conditions mediated by FXR, for example, a liver disorder, dyslipidemia and a disease related to dyslipidemia. A pharmaceutical composition can include a compound of any embodiment of Formula (I) or selected from the compounds of Table 1, or a stereoisomer, tautomer, or a pharmaceutically acceptable salt of any of the foregoing, and a pharmaceutically acceptable excipient. In some embodiments, the method of treating a disease or condition described herein in a subject in need thereof comprises administering to the subject a therapeutically effective amount of a compound of Formula (I) (including compounds of Formulae (II)-(IV)), or a stereoisomer, tautomer, or a pharmaceutically acceptable salt of any of the foregoing. In some embodiments, the method of treating a disease or condition described herein in a subject in need thereof comprises administering to the subject a therapeutically effective amount of a compound selected from compounds in Table 1, or a stereoisomer, tautomer, or a pharmaceutically acceptable salt of any of the foregoing.

Liver disorders include, without limitation, liver inflammation, fibrosis, and steatohepatitis. In some embodiments, provided herein is a method of treating a liver disorder in a subject (e.g., a human patient) in need thereof, comprising administering to the subject a therapeutically effective amount of a compound described herein, or a stereoisomer, tautomer, or a pharmaceutically acceptable salt of any of the foregoing. Exemplary liver disorders include, without limitation, liver inflammation, fibrosis, and steatohepatitis. In some embodiments, the liver disorder is selected from the list consisting of primary biliary cirrhosis (PBC), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, viral hepatitis, alcoholic liver disease, nonalcoholic fatty liver disease (NAFLD), nonalcoholic steatohepatitis (NASH), graft versus host disease, transplant liver regeneration, congenital hepatic fibrosis, choledocholithiasis, granulomatous liver disease, intra- or extrahepatic malignancy, Sjogren's syndrome, sarcoidosis, Wilson's disease, Gaucher's disease, hemochromatosis, and oti-antitrypsin deficiency. In some embodiments, the liver disorder is selected from the list consisting of liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH). In some embodiments, the liver disorder is selected from the group consisting of liver fibrosis, alcohol induced fibrosis, steatosis, alcoholic steatosis, NAFLD, and NASH. In one embodiment, the liver disorder is NASH. In another embodiment, the liver disorder is liver inflammation. In another embodiment, the liver disorder is liver fibrosis. In another embodiment, the liver disorder is alcohol induced fibrosis. In another embodiment, the liver disorder is steatosis. In another embodiment, the liver disorder is alcoholic steatosis. In another embodiment, the liver disorder is NAFLD. In one embodiment, the treatment methods provided herein impedes or slows the progression of NAFLD to NASH. In one embodiment, the treatment methods provided herein impedes or slows the progression of NASH. NASH can progress, e.g., to one or more of liver cirrhosis, hepatic cancer, etc. In some embodiments, the liver disorder is NASH. In some embodiments, the patient has had a liver biopsy. In some embodiments, the method further comprising obtaining the results of a liver biopsy.

In some embodiments, provided herein is a method of treating a liver disorder in a subject (e.g., a human patient) in need thereof, comprising administering to the subject a therapeutically effective amount of a compound described herein, or a stereoisomer, tautomer, or a pharmaceutically acceptable salt of any of the foregoing, wherein the liver disorder is selected from the group consisting of liver inflammation, liver fibrosis, alcohol induced fibrosis, steatosis, alcoholic steatosis, primary sclerosing cholangitis (PSC), primary biliary cirrhosis (PBC), non-alcoholic fatty liver disease (NAFLD), and non-alcoholic steatohepatitis (NASH).

Also provided herein are methods of impeding or slowing the progression of non-alcoholic fatty liver disease (NAFLD) to non-alcoholic steatohepatitis (NASH) in a patient (e.g., a human patient) in need thereof, comprising administering to the subject a therapeutically effective amount of a compound described herein, or a stereoisomer, tautomer, or a pharmaceutically acceptable salt of any of the foregoing.

In some embodiments, provided herein is a method of treating dyslipidemia or a disease related to dyslipidemia in a subject (e.g., a human patient) in need thereof, comprising administering to the subject a therapeutically effective amount of a compound described herein, or a stereoisomer, tautomer, or a pharmaceutically acceptable salt of any of the foregoing. In some embodiments, dyslipidemia is treated. The term “dyslipidemia” as used herein refers to an abnormality in, or abnormal amounts of lipids and lipoproteins in the blood and the disease states resulting, caused by, exacerbated by, or adjunct to such abnormality (see Dorland's Illustrated Medical Dictionary, 29th edition, W.B Saunders publishing Company, New York, N.Y.). Disease states encompassed within the definition of dyslipidemia as used herein include hyperlipidemia, hypertriglyceremia, low plasma HDL, high plasma LDL, high plasma VLDL, liver cholestosis, and hypercholesterolemia. In some embodiments, a disease related to dyslipidemia is treated. The phrase “diseases related to dyslipidemia” as used herein refers to diseases including but not limited to atherosclerosis, thrombosis, coronary artery disease, stroke, and hypertension. Diseases related to dyslipidemia also include metabolic diseases such as obesity, diabetes, insulin resistance, and complications thereof. Complications of diabetes include but are not limited diabetic retinopathy.

Further, pruritus is a well-documented adverse effect of several FXR agonists and can result in patient discomfort, a decrease in patient quality of life, and an increased likelihood of ceasing treatment. Pruritus is particularly burdensome for indications, such as those described herein, including NASH, for which chronic drug administration is likely. The tissue specificity of a compound described herein, or a stereoisomer, tautomer, or a pharmaceutically acceptable salt of any of the foregoing, in particular the preference for liver over skin tissue is a striking and unpredicted observation that makes it more likely that the compound will not cause pruritus in the skin, a theory that has been substantiated by human trials thus far.

Accordingly, provided herein are methods of treating a liver disorder in a patient in need thereof, comprising administering to the subject a therapeutically effective amount of a compound described herein, or a stereoisomer, tautomer, or a pharmaceutically acceptable salt of any of the foregoing, which preferentially distributes in liver tissue over one or more of kidney, lung, heart, and skin. In some embodiments, the administration does not result in pruritus in the patient greater than Grade 2 in severity. In some embodiments, the administration does not result in pruritus in the patient greater than Grade 1 in severity. In some embodiments, the administration does not result in pruritus in the patient. The grading of adverse effects is known. According to Version 5 of the Common Terminology Criteria for Adverse Events (published Nov. 27, 2017), Grade 1 pruritus is characterized as “Mild or localized; topical intervention indicated.” Grade 2 pruritus is characterized as “Widespread and intermittent; skin changes from scratching (e.g., edema, papulation, excoriations, lichenification, oozing/crusts); oral intervention indicated; limiting instrumental ADL.” Grade 3 pruritus is characterized as “Widespread and constant; limiting self care ADL or sleep; systemic corticosteroid or immunosuppressive therapy indicated.” Activities of daily living (ADL) are divided into two categories: “Instrumental ADL refer to preparing meals, shopping for groceries or clothes, using the telephone, managing money, etc.,” and “Self care ADL refer to bathing, dressing and undressing, feeding self, using the toilet, taking medications, and not bedridden.” Accordingly, provided herein are methods of treating a liver disorder in a patient (e.g., a human patient) in need thereof with an FXR agonist that does not result in detectable pruritus in the patient in need thereof.

In some embodiments, the patient is a human. Obesity is highly correlated with NAFLD and NASH, but lean people can also be affected by NAFLD and NASH. Accordingly, in some embodiments, the patient is obese. In some embodiments, the patient is not obese. Obesity can be correlated with or cause other diseases as well, such as diabetes mellitus or cardiovascular disorders. Accordingly, in some embodiments, the patient also has diabetes mellitus and/or a cardiovascular disorder. Without being bound by theory, it is believed that comorbidities, such as obesity, diabetes mellitus, and cardiovascular disorders can make NAFLD and NASH more difficult to treat. Conversely, the only currently recognized method for addressing NAFLD and NASH is weight loss, which would likely have little to no effect on a lean patient.

The risk for NAFLD and NASH increases with age, but children can also suffer from NAFLD and NASH, with literature reporting of children as young as 2 years old (Schwimmer, et al., Pediatrics, 2006, 118:1388-1393). In some embodiments, the patient is 2-17 years old, such as 2-10, 2-6, 2-4, 4-15, 4-8, 6-15, 6-10, 8-17, 8-15, 8-12, 10-17, or 13-17 years old. In some embodiments, the patient is 18-64 years old, such as 18-55, 18-40, 18-30, 18-26, 18-21, 21-64, 21-55, 21-40, 21-30, 21-26, 26-64, 26-55, 26-40, 26-30, 30-64, 30-55, 30-40, 40-64, 40-55, or 55-64 years old. In some embodiments, the patient is 65 or more years old, such as 70 or more, 80 or more, or 90 or more.

NAFLD and NASH are common causes of liver transplantation, but patients that already received one liver transplant often develop NAFLD and/or NASH again. Accordingly, in some embodiments, the patient has had a liver transplant.

In some embodiments, the patient's alkaline phosphatase, gamma-glutamyl transferase (GGT), alanine aminotransferase (ALT) and/or aspartate aminotransferase (AST) levels are elevated. In some embodiments, the GGT, ALT, and/or AST levels are elevated prior to treatment with a compound described herein, or a stereoisomer, tautomer, or a pharmaceutically acceptable salt of any of the foregoing. In some embodiments, the patient's ALT level is about 2-4-fold greater than the upper limit of normal levels. In some embodiments, the patient's AST level is about 2-4-fold greater than the upper limit of normal levels. In some embodiments, the patient's GGT level is about 1.5-3-fold greater than the upper limit of normal levels. In some embodiments, the patient's alkaline phosphatase level is about 1.5-3-fold greater than the upper limit of normal levels. Methods of determining the levels of these molecules are well known. Normal levels of ALT in the blood range from about 7-56 units/liter. Normal levels of AST in the blood range from about 10-40 units/liter. Normal levels of GGT in the blood range from about 9-48 units/liter. Normal levels of alkaline phosphatase in the blood range from about 53-128 units/liter for a 20- to 50-year-old man and about 42-98 units/liter for a 20- to 50-year-old woman.

Accordingly, in some embodiments, a compound described herein, or a stereoisomer, tautomer, or a pharmaceutically acceptable salt of any of the foregoing reduces level of AST, ALT, and/or GGT in an individual having elevated AST, ALT, and/or GGT levels. In some embodiments, the level of ALT is reduced at least 2-, at least 3-, at least 4-, or at least 5-fold. In some embodiments, the level of ALT is reduced about 2- to about 5-fold. In some embodiments, the level of AST is reduced at least 2-, at least 3-, at least 4-, or at least 5-fold. In some embodiments, the level of AST is reduced about 1.5 to about 3-fold. In some embodiments, the level of GGT is reduced at least 2, at least 3, at least 4, or at least 5-fold. In some embodiments, the level of GGT is reduced about 1.5 to about 3-fold.

In some embodiments, administration of a compound described herein, or a stereoisomer, tautomer, or a pharmaceutically acceptable salt of any of the foregoing to a subject results in a reduced NAFLD Activity (NAS) score. For example, in some embodiments, steatosis, inflammation, and/or ballooning is reduced upon treatment. In some embodiments, liver fibrosis is reduced. In some embodiments, serum triglycerides are reduced. In some embodiments, liver triglycerides are reduced.

In some embodiments, the patient is at risk of developing an adverse effect prior to the administration in accordance with the methods provided herein. In some embodiments, the adverse effect is an adverse effect which affects the kidney, lung, heart, and/or skin. In some embodiments, the adverse effect is pruritus.

In some embodiments, the patient has had one or more prior therapies. In some embodiments, the liver disorder progressed during the therapy. In some embodiments, the patient suffered from pruritus during at least one of the one or more prior therapies. In some embodiments, the methods described herein do not comprise treating pruritus in the patient.

In some embodiments, the therapeutically effective amount is below the level that induces an adverse effect in the patient, such as below the level that induces pruritus, such as grade 2 or grade 3 pruritus.

Also provided herein are dosing regimens for administering a compound described herein, or a stereoisomer, tautomer, or a pharmaceutically acceptable salt of any of the foregoing to an individual in need thereof. In some embodiments, the therapeutically effective amount of a compound described herein, or a stereoisomer, tautomer, or a pharmaceutically acceptable salt of any of the foregoing is 500 μg/day-600 mg/day. In some embodiments, the therapeutically effective amount is 500 μg/day-300 mg/day. In some embodiments, the therapeutically effective amount is 500 μg/day-150 mg/day. In some embodiments, the therapeutically effective amount is 500 μg/day-100 mg/day. In some embodiments, the therapeutically effective amount is 500 μg/day-20 mg/day. In some embodiments, the therapeutically effective amount is 1 mg/day-600 mg/day. In some embodiments, the therapeutically effective amount is 1 mg/day-300 mg/day. In some embodiments, the therapeutically effective amount is 1 mg/day-150 mg/day. In some embodiments, the therapeutically effective amount is 1 mg/day-100 mg/day. In some embodiments, the therapeutically effective amount is 1 mg/day-20 mg/day. In some embodiments, the therapeutically effective amount is 5 mg/day-300 mg/day. In some embodiments, the therapeutically effective amount is 5 mg/day-150 mg/day. In some embodiments, the therapeutically effective amount is 5 mg/day-100 mg/day. In some embodiments, the therapeutically effective amount is 5 mg/day-20 mg/day. In some embodiments, the therapeutically effective amount is 5 mg/day-15 mg/day. In some embodiments, the therapeutically effective amount is 10 mg/day-300 mg/day. In some embodiments, the therapeutically effective amount is 10 mg/day-150 mg/day. In some embodiments, the therapeutically effective amount is 10 mg/day-100 mg/day. In some embodiments, the therapeutically effective amount is 10 mg/day-30 mg/day. In some embodiments, the therapeutically effective amount is 10 mg/day-20 mg/day. In some embodiments, the therapeutically effective amount is 10 mg/day-15 mg/day. In some embodiments, the therapeutically effective amount is 25 mg/day-300 mg/day. In some embodiments, the therapeutically effective amount is 25 mg/day-150 mg/day. In some embodiments, the therapeutically effective amount is 25 mg/day-100 mg/day. In some embodiments, the therapeutically effective amount is 500 μg/day-5 mg/day. In some embodiments, the therapeutically effective amount is 500 μg/day-4 mg/day. In some embodiments, the therapeutically effective amount is 5 mg/day-600 mg/day. In another embodiment, the therapeutically effective amount is 75 mg/day-600 mg/day.

The dosage amount of a compound as described herein is determined based on the free base of a compound. In some embodiments, about 1 mg to about 30 mg of a compound described herein, or a stereoisomer, tautomer, or a pharmaceutically acceptable salt of any of the foregoing is administered to the individual. In some embodiments, about 1 mg to about 5 mg is administered to the individual. In some embodiments, about 1 mg to about 3 mg is administered to the individual. In some embodiments, about 5 mg to about 10 mg is administered to the individual. In some embodiments, about 10 mg to about 15 mg is administered to the individual. In some embodiments, about 15 mg to about 20 mg is administered to the individual. In some embodiments, about 20 mg to about 25 mg is administered to the individual. In some embodiments, about 25 mg to about 30 mg is administered to the individual. In some embodiments, about 1 mg is administered to the individual. In some embodiments, about 2 mg is administered to the individual. In some embodiments, about 3 mg is administered to the individual. In some embodiments, about 4 mg is administered to the individual. In some embodiments, about 5 mg is administered to the individual. In some embodiments, about 6 mg is administered to the individual. In some embodiments, about 7 mg is administered to the individual. In some embodiments, about 8 mg is administered to the individual. In some embodiments, about 9 mg is administered to the individual. In some embodiments, about 10 mg is administered to the individual. In some embodiments, about 15 mg is administered to the individual. In some embodiments, about 20 mg is administered to the individual. In some embodiments, about 25 mg is administered to the individual. In some embodiments, about 30 mg is administered to the individual.

The treatment period generally can be one or more weeks. In some embodiments, the treatment period is at least 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more. In some embodiments, the treatment period is from about a week to about a month, from about a month to about a year, from about a year to about several years. In some embodiments, the treatment period at least any of about 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more. In some embodiments, the treatment period is the remaining lifespan of the patient.

The administration can be once daily, twice daily or every other day, for a treatment period of one or more weeks. In some embodiments, the administration comprises administering compounds and/or compositions described herein daily for a treatment period of one or more weeks. In some embodiments, the administration comprises administering compounds and/or compositions described herein twice daily for a treatment period of one or more weeks. In some embodiments, the administration comprises administering compounds and/or compositions described herein every other day for a treatment period of one or more weeks.

In some embodiments, compounds and/or compositions described herein are administered to the individual once per day for at least seven days, wherein the daily amounts are independently in a range of about 1 mg to about 10 mg, about 1 mg to about 5 mg or about 1 mg to about 3 mg, or about any one of 1, 2, 3, 4, 5, 6, 7, 8, 9, or 10 mg. In some embodiments, compounds and/or compositions described herein are administered to the individual once per day for at least 14 days, wherein the daily amounts are independently in a range of about 1 mg to about 10 mg, about 1 mg to about 5 mg or about 1 mg to about 3 mg or about any one of 1, 2, 3, 4, 5, 6, 7, 8, 9, or 10 mg. In some embodiments, compounds and/or compositions described herein are administered to the individual once per day for a period of between one and four weeks, wherein the daily amounts are independently in a range of about 1 mg to about 10 mg, about 1 mg to about 5 mg or about 1 mg to about 3 mg or about any one of 1, 2, 3, 4, 5, 6, 7, 8, 9, or 10 mg.

In accordance with the present application, compounds and/or compositions described herein can be administered by any suitable route in the form of a pharmaceutical composition adapted to such a route, and in a dose effective for the treatment intended. The compounds and/or compositions described herein may be administered orally, rectally, vaginally, parenterally, or topically.

In some embodiments, the compounds and/or compositions may be administered orally. Oral administration may involve swallowing, so that the compound enters the gastrointestinal tract, or buccal or sublingual administration may be employed by which the compound enters the bloodstream directly from the mouth.

In some embodiments, the compounds and/or compositions may be administered directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

In some embodiments, the compounds and/or compositions may be administered topically to the skin or mucosa, that is, dermally or transdermally. In some embodiments, the compounds and/or compositions may be administered intranasally or by inhalation. In some embodiments, the compounds and/or compositions may be administered rectally or vaginally. In some embodiments, the compounds and/or compositions may be administered directly to the eye or ear.

The dosage regimen for the compounds and/or compositions described herein is based on a variety of factors, including the type, age, weight, sex and medical condition of the patient; the severity of the condition; the route of administration; and the activity of the particular compound employed. Thus the dosage regimen may vary widely. In some embodiments, the total daily dose of the compounds of the present application is typically from about 0.001 to about 100 mg/kg (i.e., mg compound per kg body weight) for the treatment of the indicated conditions discussed herein. In one embodiment, total daily dose of the compounds of the present application is from about 0.01 to about 30 mg/kg, and in another embodiment, from about 0.03 to about 10 mg/kg, and in yet another embodiment, from about 0.1 to about 3. It is not uncommon that the administration of the compounds of the present application will be repeated a plurality of times in a day (typically no greater than 4 times). Multiple doses per day typically may be used to increase the total daily dose, if desired.

For oral administration, the compounds and/or compositions described herein may be provided in the form of tablets containing 0.1, 0.5, 1.0, 2.5, 5.0, 10.0, 15.0, 25.0, 30.0 50.0, 75.0, 100, 125, 150, 175, 200, 250 and 500 milligrams of the active ingredient for the symptomatic adjustment of the dosage to the patient. A medicament typically contains from about 0.01 mg to about 500 mg of the active ingredient, or in another embodiment, from about 1 mg to about 100 mg of active ingredient. Intravenously, doses may range from about 0.01 to about 10 mg/kg/minute during a constant rate infusion.

The compounds and/or compositions described herein can be used alone, or in combination with other therapeutic agents. The administration of two or more agents “in combination” means that all of the agents are administered closely enough in time that each may generate a biological effect in the same time frame. The presence of one agent may alter the biological effects of the other agent(s). The two or more agents may be administered simultaneously, concurrently or sequentially. Additionally, simultaneous administration may be carried out by mixing the agents prior to administration or by administering the compounds at the same point in time but as separate dosage forms at the same or different site of administration.

The present application provides any of the uses, methods or compositions as defined herein wherein a compound described herein, or a stereoisomer, tautomer, or a pharmaceutically acceptable salt of any of the foregoing is used in combination with one or more other therapeutic agent. This would include a pharmaceutical composition comprising a compound described herein, or a stereoisomer, tautomer, or a pharmaceutically acceptable salt of any of the foregoing, as defined in any of the embodiments described herein, in admixture with at least one pharmaceutically acceptable excipient and one or more other therapeutic agent.

In some embodiments, the one or more other therapeutic agent is an agent to treat NASH including but not limited to PF-05221304, an FXR agonist (e.g., obeticholic acid), a PPAR a/d agonist (e.g., elafibranor), a synthetic fatty acid-bile acid conjugate (e.g., aramchol), a caspase inhibitor (e.g., emricasan), an anti-lysyl oxidase homologue 2 (LOXL2) monoclonal antibody (e.g., simtuzumab), a galectin 3 inhibitor (e.g., GR-MD-02), a MAPK5 inhibitor (e.g., GS-4997), a dual antagonist of chemokine receptor 2 (CCR2) and CCR5 (e.g., cenicriviroc), a fibroblast growth factor2l (FGF21) agonist (e.g., BMS-986036), a leukotriene D4 (LTD4) receptor antagonist (e.g., tipelukast), a niacin analogue (e.g., ARI 3037MO), an ASBT inhibitor (e.g., volixibat), an acetyl-CoA carboxylase (ACC) inhibitor (e.g., NDI 010976), a ketohexokinase (KHK) inhibitor, a diacylglyceryl acyltransferase 2 (DGAT2) inhibitor, a CB1 receptor antagonist, an anti-CB1 R antibody, or an apoptosis signal-regulating kinase 1 (ASK1) inhibitor, including the pharmaceutically acceptable salts of the specifically named agents and the pharmaceutically acceptable solvates of said agents and salts.

### Articles of Manufacture and Kits

The present disclosure further provides articles of manufacture comprising a compound, or a stereoisomer, tautomer, or a pharmaceutically acceptable salt of any of the foregoing in accordance with the present application, a composition described herein, or one or more unit dosages described herein in suitable packaging. In certain embodiments, the article of manufacture is for use in any of the methods described herein. Suitable packaging (e.g., containers) is known in the art and includes, for example, vials, vessels, ampules, bottles, jars, flexible packaging and the like. An article of manufacture may further be sterilized and/or sealed.

The kits may be in unit dosage forms, bulk packages (e.g., multi-dose packages) or sub-unit doses. For example, kits may be provided that contain sufficient dosages of a compound, or a stereoisomer, tautomer, or a pharmaceutically acceptable salt of any of the foregoing in accordance with the present application, a composition described herein, and/or one or more other therapeutic agent useful for a disease detailed herein to provide effective treatment of an individual for an extended period, such as any of a week, 2 weeks, 3 weeks, 4 weeks, 6 weeks, 8 weeks, 3 months, 4 months, 5 months, 7 months, 8 months, 9 months, or more. Kits may also include multiple unit doses of the compounds/compositions described herein and instructions for use and be packaged in quantities sufficient for storage and use in pharmacies (e.g., hospital pharmacies and compounding pharmacies).

The kits may optionally include a set of instructions, generally written instructions, although electronic storage media (e.g., magnetic diskette or optical disk) containing instructions are also acceptable, relating to the use of component(s) of the methods of the present disclosure. The instructions included with the kit generally include information as to the components and their administration to an individual.

### EXAMPLES

### Synthetic Examples

As will be readily appreciated, compounds described herein can be readily prepared by methods that are well-known and established in the art including methods and procedures similar to those described herein. For example, U.S. Pat. No. 8,153,624 discloses general reactions as depicted in Schemes 1, 2, 3 and 4, for preparation of compounds purportedly useful as farnesoid X receptor (FXR), which is incorporated herein by reference.

### Example 1

### 3-(4-((5-cyclopropyl-3-(2,6-dichlorophenyl)isoxazol-4-yl)methoxy)piperidine-1-carbonyl)-1,2,4-oxadiazol-5(4H)-one

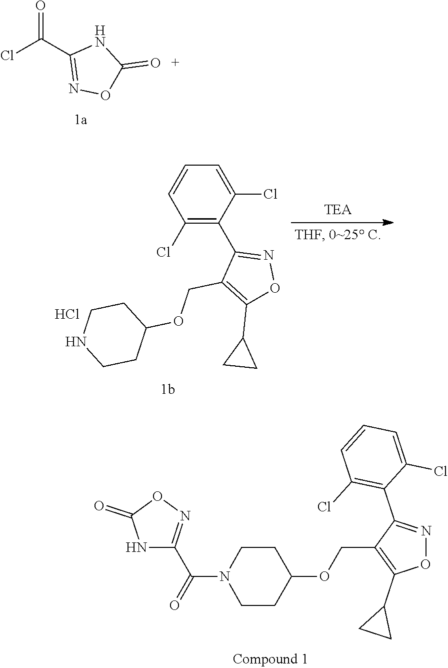

3-(4-((5-cyclopropyl-3-(2,6-dichlorophenyl)isoxazol-4-yl)methoxy)piperidine-1-carbonyl)-1,2,4-oxadiazol-5(4H)-one (Compound 1). To a solution of 5-cyclopropyl-3-(2,6-dichlorophenyl)-4-((piperidin-4-yloxy)methyl)isoxazole hydrochloride (1b) (50 mg, 123.85 umol) and TEA (50.13 mg, 495.38 umol, 68.95 uL) in THF (0.5 mL) was cooled to 0° C. and then 5-oxo-4,5-dihydro-1,2,4-oxadiazole-3-carbonyl chloride (1a) (1.5 M, 123.85 uL) was added dropwise at 0° C. The reaction mixture was stirred for 50 minutes at 25° C. The reaction was adjusted pH to 7 with 1 N HCl solution and extracted with ethyl acetate (5 mL×2). The organic layers were combined and concentrated under reduced pressure to give a residue. The residue was purified by flash silica gel chromatography (column: Welch Ultimate AQ-C18 150*30 mm*5 um; mobile phase: [water (0.10% TFA)-ACN]; B %: 42%-72%, 12 min) to give Compound 1. MS mass calculated for [M+H]⁺(C₂₁H₂OCl₂N₄O₅) requires m z, 479.1/481.1, LCMS found m z 479.1/481.1; ¹H NMR (400 MHz, CHLOROFORM-d) δ=7.47-7.40 (m, 2H), 7.38-7.33 (m, 1H), 4.33 (s, 2H), 4.08-3.96 (m, 1H), 3.86-3.67 (m, 2H), 3.58 (td, J=2.7, 5.8 Hz, 1H), 3.48 (ddd, J=3.5, 9.3, 13.1 Hz, 1H), 2.12 (tt, J=5.1, 8.4 Hz, 1H), 1.75-1.55 (m, 4H), 1.31-1.23 (m, 2H), 1.17-1.09 (m, 2H).

### Example 2

### 4-(((1-(4-(1H-diazirin-3-yl)phenyl)piperidin-4-yl)oxy)methyl)-5-cyclopropyl-3-(2,6-dichlorophenyl)isoxazole

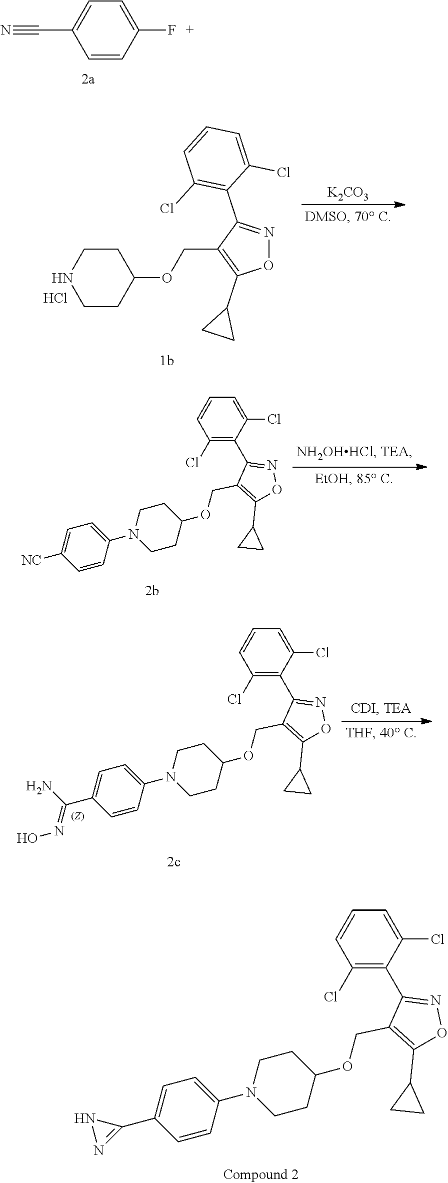

4-(4-((5-cyclopropyl-3-(2,6-dichlorophenyl)isoxazol-4-yl)methoxy)piperidin-1-yl)benzonitrile (2b). To a solution of 4-fluorobenzonitrile (2a) (89.99 mg, 743.07 umol) in DMSO (2 mL) was added K₂CO₃ (136.93 mg, 990.76 umol) and 5-cyclopropyl-3-(2,6-dichlorophenyl)-4-((piperidin-4-yloxy)methyl)isoxazole hydrochloride (1b) (100 mg, 247.69 umol) at 25° C., and the mixture was heated to 70° C. for 16 hours. The reaction mixture was poured into H₂O (10 mL) and extracted with ethyl acetate (20 mL*2), the organic layer was washed with brine (10 mL), dried over anhydrous Na₂SO₄, filtered and concentrated under reduced pressure to give a residue. The residue was purified by prep-TLC (SiO₂, petroleum ether:ethyl acetate=2:1) to give 2b. MS mass calculated for [M+H]⁺(C₂₅H₂₃Cl₂N₃O₂) requires m/z, 468.1/470.1, LCMS found m/z, 468.1/470.1.

(Z)-4-(4-((5-cyclopropyl-3-(2,6-dichlorophenyl)isoxazol-4-yl)methoxy)piperidin-1-yl)-N′-hydroxybenzimidamide (2c). To a solution of 4-(4-((5-cyclopropyl-3-(2,6-dichlorophenyl)isoxazol-4-yl)methoxy)piperidin-1-yl)benzonitrile (2b) (90 mg, 192.15 umol) in ethanol (5 mL) was added hydroxylamine hydrochloride (26.71 mg, 384.31 umol) and TEA (38.89 mg, 384.31 umol) at 25° C., and the mixture was stirred at 85° C. for 16 hours. The reaction mixture was poured into H₂O (10 mL) and extracted with ethyl acetate (20 mL*2), the organic layer was washed with brine (10 mL), dried over anhydrous Na₂SO₄, filtered and concentrated under reduced pressure to give a residue. The residue was purified by prep-TLC (SiO₂, dichloromethane:Methanol=20:1) to give 2c. MS mass calculated for [M+H]⁺(C₂₅H₂₆Cl₂N₄O₃) requires m/z, 501.1/503.1, LCMS found m/z, 501.2/503.2; ¹H NMR (400 MHz, DMSO-d6) δ=9.32 (s, 1H), 7.63-7.58 (m, 2H), 7.55-7.50 (m, 1H), 7.48 (d, J=8.8 Hz, 2H), 6.83 (br d, J=8.9 Hz, 2H), 5.61 (s, 2H), 4.31 (s, 2H), 3.25 (br s, 1H), 2.84 (br t, J=9.2 Hz, 2H), 2.37-2.30 (m, 1H), 1.68 (br s, 2H), 1.37-1.27 (m, 2H), 1.18-1.12 (m, 2H), 1.11-1.07 (m, 2H).

4-(((1-(4-(1H-diazirin-3-yl) phenyl) piperidin-4-yl) oxy) methyl)-5-cyclopropyl-3-(2, 6-dichlorophenyl)isoxazole (Compound 2). To a solution of (Z)-4-(4-((5-cyclopropyl-3-(2,6-dichlorophenyl)isoxazol-4-yl)methoxy)piperidin-1-yl)-N′-hydroxybenzimidamide (2c) (50 mg, 99.72 umol) in THE (2 mL) was added CDI (32.34 mg, 199.44 umol) and TEA (20.18 mg, 199.44 umol) at 25° C. The mixture was heated to 40° C. for 1 hour. The reaction mixture was poured into H₂O (10 mL) and extracted with ethyl acetate (20 mL*2), the organic layer was washed with brine (10 mL), dried over anhydrous Na₂SO₄, filtered and concentrated under reduced pressure to give a residue. The residue was purified by prep-TLC (SiO₂, petroleum ether:ethyl acetate=1:1) to give Compound 2. MS mass calculated for [M+H]⁺(C₂₅H₂₄Cl₂N₄O₂) requires m/z, 483.1/485.1, LCMS found m/z, 483.2/485.2; ¹H NMR (400 MHz, CHLOROFORM-d) δ=7.42-7.36 (m, 2H), 7.33-7.28 (m, 1H), 6.94-6.81 (m, 4H), 4.34 (s, 2H), 3.39 (td, J=4.0, 7.9 Hz, 1H), 3.24-3.13 (m, 2H), 2.79 (ddd, J=3.1, 8.8, 12.0 Hz, 2H), 2.16 (tt, J=5.1, 8.4 Hz, 1H), 1.84-1.76 (m, 2H), 1.62-1.54 (m, 2H), 1.28 (dd, J=2.4, 5.0 Hz, 2H), 1.16-1.10 (m, 2H).

### Example 3

### 3-(4-(4-((5-cyclopropyl-3-(2,6-dichlorophenyl)isoxazol-4-yl)methoxy)piperidin-1-yl)phenyl)-1,2,4-oxadiazol-5(4H)-one

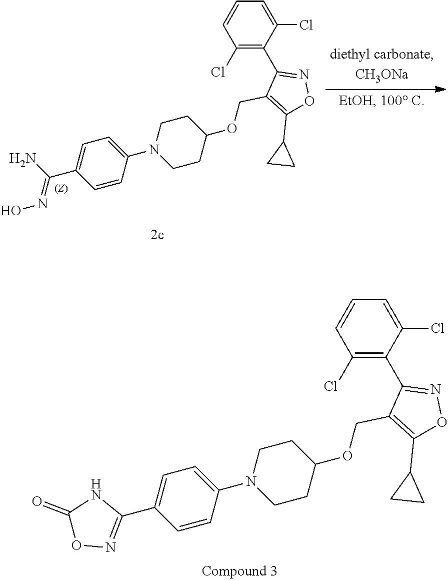 [SMILES: N/C(=N\O)c1ccc(N2CCC(OCc3c(-c4c(Cl)cccc4Cl)noc3C3CC3)CC2)cc1 ; O=c1[nH]c(-c2ccc(N3CCC(OCc4c(-c5c(Cl)cccc5Cl)noc4C4CC4)CC3)cc2)no1]

3-(4-(4-((5-cyclopropyl-3-(2,6-dichlorophenyl)isoxazol-4-yl)methoxy)piperidin-1-yl)phenyl)-1,2,4-oxadiazol-5(4H)-one (Compound 3). To a solution of 4-(4-((5-cyclopropyl-3-(2,6-dichlorophenyl)isoxazol-4-yl)methoxy)piperidin-1-yl)-N-hydroxybenzimidamide (2c) (200 mg, 398.88 umol) in ethanol (10 mL) was added CH₃ONa (517.18 mg, 2.39 mmol, 30% in MeOH) and diethyl carbonate (376.96 mg, 3.19 mmol) at 25° C. The mixture was heated to 100° C. for 40 hours. The reaction mixture was poured into H₂O (10 mL) and extracted with ethyl acetate (30 mL*2). The combined organic layer was washed with brine (10 mL), dried over anhydrous Na₂SO₄, filtered and concentrated under reduced pressure to give a residue. The residue was purified by prep-TLC (SiO₂, petroleum ether:ethyl acetate=1:1) to give Compound 3. MS mass calculated for [M+H]⁺(C₂₆H₂₄Cl₂N₄O₄) requires m/z, 527.1/529.1, LCMS found m/z, 527.0/529.0; ¹H NMR (400 MHz, CHLOROFORM-d) δ=7.67-7.57 (m, 2H), 7.40 (br d, J=7.8 Hz, 2H), 7.31 (br d, J=7.8 Hz, 1H), 6.89 (br d, J=8.4 Hz, 2H), 4.36 (s, 2H), 3.49 (br s, 1H), 3.38 (br s, 2H), 3.13-3.03 (m, 2H), 2.16 (br s, 1H), 1.78 (br s, 2H), 1.57 (br s, 2H), 1.28 (br s, 2H), 1.14 (br d, J=5.6 Hz, 2H).

### Example 4

### 4-(6-(4-((5-cyclopropyl-3-(2,6-dichlorophenyl)isoxazol-4-yl)methoxy)piperidin-1-yl)pyridin-3-yl)-1,2,4-triazine-3, 5(2H,4H)-dione

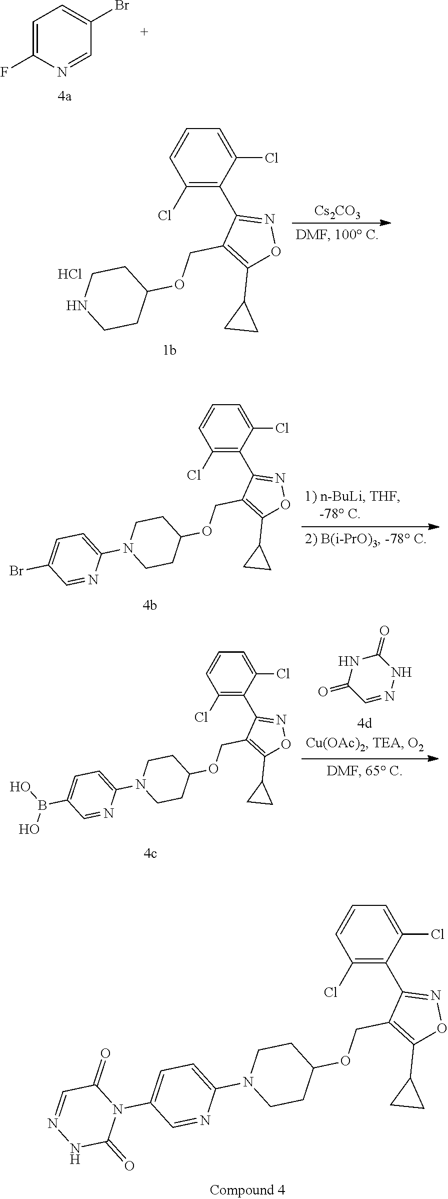

4-(((1-(5-bromopyridin-2-yl)piperidin-4-yl)oxy)methyl)-5-cyclopropyl-3-(2,6-dichlorophenyl)isoxazole (4b). To a solution of 5-bromo-2-fluoropyridine (4a) (156.92 mg, 891.69 umol, 91.77 uL) and 5-cyclopropyl-3-(2,6-dichlorophenyl)-4-((piperidin-4-yloxy)methyl)isoxazole hydrochloride (1b) (300 mg, 743.07 umol) in DMF (3 mL) was added Cs₂CO₃ (532.63 mg, 1.63 mmol) at 20° C. The mixture was stirred at 100° C. for 2 hours. The reaction mixture was then poured into water (10 mL) and extracted with ethyl acetate (10 mL*3). The organic layers were combined and washed with brine (10 mL*2), dried over anhydrous Na₂SO₄ and filtered, the filtrate was concentrated under reduced pressure. The residue was purified by prep-TLC (Petroleum ether:ethyl acetate=5:1) to give 4b. MS mass calculated for [M+H]⁺(C₂₃H₂₂BrCl₂N₃O₂) requires m/z, 524.0/522.0/526.1, LCMS found m/z, 524.0/521.9/526.0; ¹H NMR (CHLOROFORM-d, 400 MHz): δ=8.08 (d, J=2.1 Hz, 1H), 7.41 (dd, J=9.0, 2.6 Hz, 1H), 7.33 (d, J=1.1 Hz, 1H), 7.31 (s, 1H), 7.20-7.26 (m, 1H), 6.43 (d, J=9.0 Hz, 1H), 4.27 (s, 2H), 3.51-3.64 (m, 2H), 3.38 (tt, J=7.7, 3.7 Hz, 1H), 2.97-3.13 (m, 2H), 2.08 (tt, J=8.4, 5.1 Hz, 1H), 1.61-1.70 (m, 2H), 1.32-1.45 (m, 2H), 1.20 (dd, J=5.0, 2.5 Hz, 2H), 1.01-1.09 (m, 2H).

(6-(4-((5-cyclopropyl-3-(2,6-dichlorophenyl)isoxazol-4-yl)methoxy)piperidin-1-yl)pyridin-3-yl)boronic acid (4c). To a solution of 4-(((1-(5-bromopyridin-2-yl)piperidin-4-yl)oxy)methyl)-5-cyclopropyl-3-(2,6-dichlorophenyl)isoxazole (4b) (220 mg, 420.45 umol) in THF (5 mL) was added n-BuLi (2.5 M, 218.63 uL) dropwise at −78° C. After 30 min, triisopropyl borate (158.15 mg, 840.90 umol, 193.34 uL) was added to the mixture at −78° C. and the mixture was stirred at −78° C. for 2 hours. The reaction mixture was quenched with NH₄Cl solution (0.5 mL), water (10 mL) and extracted with ethyl acetate (10 mL*3). The combined organic layers were washed with brine (10 ml), dried over anhydrous Na₂SO₄, filtered and concentrated to give a residue. The residue was purified by prep-HPLC (column: Waters Xbridge BEH C18 100*30 mm*10 um; mobile phase: [water (10 mM NH₄HCO₃)-ACN]; B %: 30%-60%, 10 min) to give 4c. MS mass calculated for [M+H]⁺(C₂₃H₂₄BCl₂N₃O₄) requires m/z, 488.1/490.1, LCMS found m/z, 488.1/490.0.

4-(6-(4-((5-cyclopropyl-3-(2,6-dichlorophenyl)isoxazol-4-yl)methoxy)piperidin-1-yl)pyridin-3-yl)-1,2,4-triazine-3,5(2H,4H)-dione (Compound 4). To a solution of (6-(4-((5-cyclopropyl-3-(2,6-dichlorophenyl)isoxazol-4-yl)methoxy)piperidin-1-yl)pyridin-3-yl)boronic acid (4c) (20 mg, 40.97 umol) in DMF (1 mL) was added 1,2,4-triazine-3,5(2H,4H)-dione (4d) (4.63 mg, 40.97 umol), Cu(OAc)₂ (7.44 mg, 40.97 umol), TEA (8.29 mg, 81.94 umol, 11.40 uL) and 4 A MS (50 mg) at 25° C. The suspension was degassed under vacuum and purged with O₂ 3 times. The mixture was stirred under O₂ ballon at 65° C. for 4 hours and acetonitrile (1 mL) was added. The mixture was filtered and the filtrate was concentrated under reduced pressure. The residue was purified by prep-HPLC (column: Phenomenex Luna C18 150*30 mm*5 um; mobile phase: [water (0.1% TFA)-ACN]; B %: 35%-65%, 10 min) to give Compound 4. MS mass calculated for [M+H]⁺(C₂₆H₂₄Cl₂N₆O₄) requires m/z, 555.1/557.1, LCMS found m/z, 555.0/557.0; ¹H NMR (400 MHz, CHLOROFORM-d) δ=10.62 (s, 1H) 8.35 (s, 1H) 7.50-7.62 (m, 1H) 7.40-7.49 (m, 3H) 7.31-7.39 (m, 1H) 6.92 (d, J=9.3 Hz, 1H) 4.36 (s, 2H) 3.60 (d, J=4.9 Hz, 5H) 2.08-2.21 (m, 1H) 1.77 (s, 2H) 1.68 (d, J=5.0 Hz, 2H) 1.25-1.33 (m, 2H) 1.09-1.20 (m, 2H).

### Example 5

### 3-(2-(4-((5-cyclopropyl-3-(2,6-dichlorophenyl)isoxazol-4-yl)methoxy)piperidin-1-yl)pyrimidin-5-yl)-1,2,4-oxadiazol-5(4H)-one

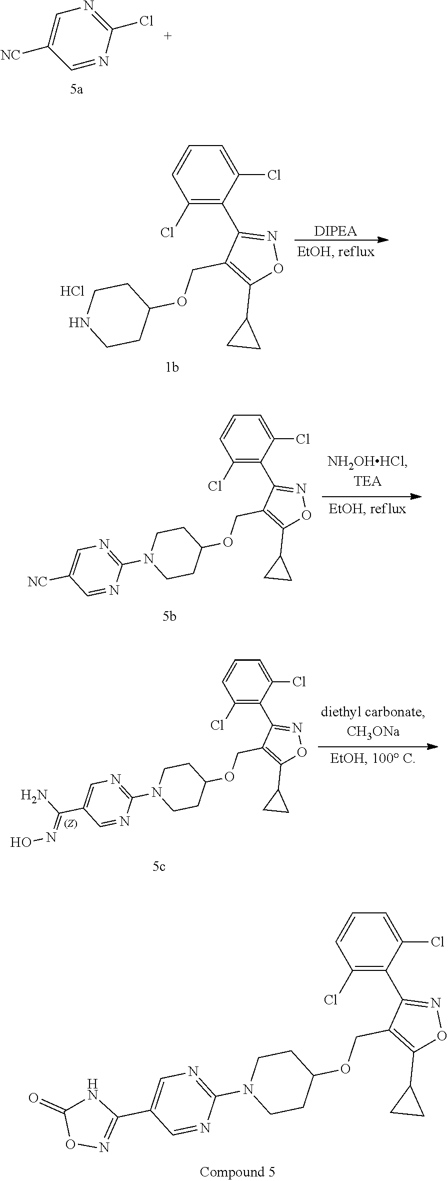

2-(4-((5-cyclopropyl-3-(2,6-dichlorophenyl)isoxazol-4-yl)methoxy)piperidin-1-yl)pyrimidine-5-carbonitrile (5b). To a solution of 2-chloropyrimidine-5-carbonitrile (5a) (20.74 mg, 148.61 umol) in ethanol (2 mL) was added 5-cyclopropyl-3-(2,6-dichlorophenyl)-4-((piperidin-4-yloxy)methyl)isoxazole hydrochloride (1b) (50 mg, 123.85 umol) and DIPEA (32.01 mg, 247.69 umol, 43.14 uL) at 25° C. The mixture was degassed and purged with N₂ 3 times then was heated to reflux and stirred for 18 hours. The reaction mixture was diluted with water (5 mL) and extracted with ethyl acetate (5 mL*2). The combined organic layer was washed with brine (5 mL), dried over anhydrous Na₂SO₄, filtered and concentrated under reduced pressure to give a residue. The residue was purified by prep-TLC to give 5b. MS mass calculated for [M+H]⁺(C₂₃H₂₁Cl₂N₅O₂) requires m/z, 470.1/472.1, LCMS found m/z, 470.1, 471.8; ¹H NMR (400 MHz, CHLOROFORM-d) δ=8.46 (s, 2H), 7.43 (d, J=1.0 Hz, 1H), 7.41 (s, 1H), 7.38-7.30 (m, 1H), 4.36 (s, 2H), 3.95-3.81 (m, 2H), 3.72-3.60 (m, 2H), 3.58-3.48 (m, 1H), 2.20-2.12 (m, 1H), 1.75-1.65 (m, 2H), 1.50 (dtd, J=3.9, 7.1, 13.7 Hz, 2H), 1.30-1.26 (m, 2H), 1.18-1.10 (m, 2H).

(Z)-2-(4-((5-cyclopropyl-3-(2,6-dichlorophenyl)isoxazol-4-yl)methoxy)piperidin-1-yl)-N′-hydroxypyrimidine-5-carboximidamide (5c). To a solution of 2-(4-((5-cyclopropyl-3-(2,6-dichlorophenyl)isoxazol-4-yl)methoxy)piperidin-1-yl)pyrimidine-5-carbonitrile (5b) (110 mg, 233.87 umol) in ethanol (5 mL) was added TEA (47.33 mg, 467.74 umol, 65.10 uL) and hydroxylamine hydrochloride (32.50 mg, 467.74 umol) at 25° C. The mixture was degassed and purged with N₂ for 3 times and was heated to reflux and stirred for 18 hours. The reaction mixture was diluted with water (5 mL) and extracted with ethyl acetate (5 mL*3). The combined organic layers were washed with brine (5 mL), dried over anhydrous Na₂SO₄, filtered and concentrated under reduced pressure to give a residue. The residue was purified by prep-TLC to give 5c. MS mass calculated for [M+H]⁺(C₂₃H₂₄Cl₂N₆O₃) requires m/z, 503.1, 505.1, LCMS found m/z, 503.1, 504.9; ¹H NMR (400 MHz, CHLOROFORM-d) δ=8.50 (s, 2H), 7.43 (d, J=1.0 Hz, 1H), 7.41 (s, 1H), 7.36-7.30 (m, 1H), 4.80-4.71 (m, 2H), 4.36 (s, 2H), 4.00 (ddd, J=3.7, 7.1, 13.2 Hz, 2H), 3.55-3.43 (m, 3H), 2.20-2.12 (m, 1H), 1.76-1.68 (m, 2H), 1.45 (dq, J=3.9, 8.3 Hz, 2H), 1.31-1.25 (m, 2H), 1.18-1.10 (m, 2H).

3-(2-(4-((5-cyclopropyl-3-(2,6-dichlorophenyl)isoxazol-4-yl)methoxy)piperidin-1-yl)pyrimidin-5-yl)-1,2,4-oxadiazol-5(4H)-one (Compound 5). To a solution of (Z)-2-(4-((5-cyclopropyl-3-(2,6-dichlorophenyl)isoxazol-4-yl)methoxy)piperidin-1-yl)-N′-hydroxypyrimidine-5-carboximidamide (5c) (40 mg, 79.46 umol) in ethanol (1 mL) was added NaOMe (100.17 mg, 556.24 umol, 30% in MeOH) and diethyl carbonate (103.26 mg, 874.09 umol, 105.90 uL) in a sealed tube. The reaction mixture was stirred at 100° C. for 18 hours and was cooled to 25° C. Another batch of NaOMe (100.17 mg, 556.24 umol, 30% in MeOH) and diethyl carbonate (103.26 mg, 874.09 umol, 105.90 uL) were added. The mixture was heated to 100° C. again and stirred for another 24 hours. The reaction mixture was diluted with water (5 mL) and extracted with ethyl acetate (5 mL*2). The combined organic layer was washed with brine (5 mL), dried over anhydrous Na₂SO₄, filtered and concentrated under reduced pressure to give a residue. The residue was purified by prep-HPLC (neutral condition: column: Waters Xbridge BEH C18 100*30 mm*10 um; mobile phase: [water (10 mM NH₄HCO₃)-ACN]; B %: 25%-50%, 8 min) and lyophilized to give Compound 5. MS mass calculated for [M+H]⁺(C₂₄H₂₂Cl₂N₆O₄) requires m/z, 529.1, 531.1, LCMS found m/z, 529.0, 531.0; ¹H NMR (400 MHz, CHLOROFORM-d) δ=8.61 (s, 2H), 7.43 (s, 1H), 7.42 (s, 1H), 7.38-7.31 (m, 1H), 4.37 (s, 2H), 3.95 (br s, 2H), 3.62 (br s, 2H), 3.54 (br s, 1H), 2.21-2.12 (m, 1H), 1.71 (br s, 2H), 1.50 (br s, 2H), 1.33-1.24 (m, 2H), 1.19-1.09 (m, 2H).

### Example 6

### 3-(6-(4-((5-cyclopropyl-3-(2,6-dichlorophenyl)isoxazol-4-yl)methoxy)piperidin-1-yl)pyridin-3-yl)-1,2,4-oxadiazol-5(4H)-one

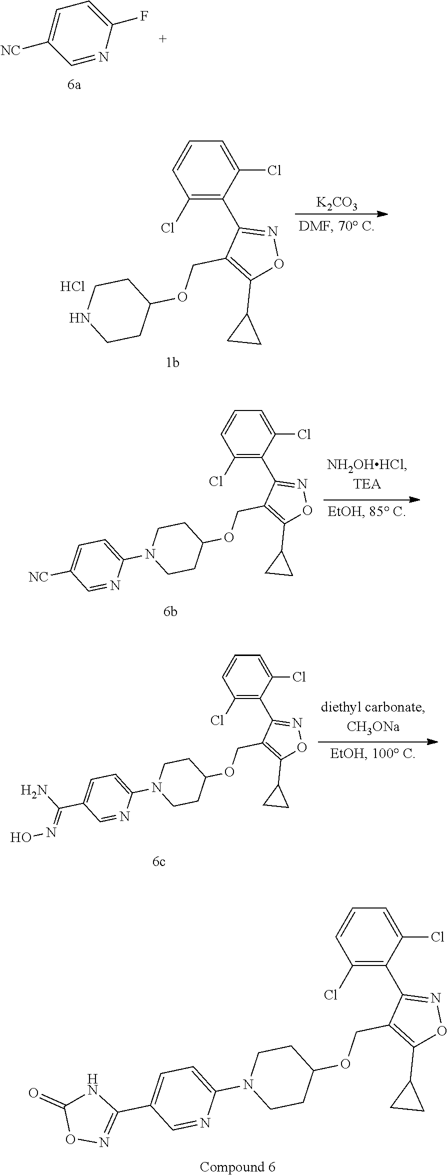

6-(4-((5-cyclopropyl-3-(2,6-dichlorophenyl)isoxazol-4-yl)methoxy)piperidin-1-yl)nicotinonitrile (6b). To a solution of 5-cyclopropyl-3-(2,6-dichlorophenyl)-4-((piperidin-4-yloxy)methyl)isoxazole hydrochloride (1b) (137.77 mg, 341.25 umol) in DMF (1 mL) was added K₂CO₃ (141.49 mg, 1.02 mmol) at 25° C. and the mixture was stirred at 25° C. for 10 minutes. 6-Fluoronicotinonitrile (6a) (50 mg, 409.50 umol) was added to the mixture at 25° C., the mixture was degassed and purged with N₂ 3 times. The reaction mixture was stirred at 70° C. for 16 hour under N₂ and was poured into H₂O (10 mL) and was extracted with ethyl acetate (20 mL*2). The combined organic layers were washed with brine (10 mL), dried over anhydrous Na₂SO₄, filtered and concentrated under reduced pressure to give a residue. The residue was purified by prep-TLC to give 6b. MS mass calculated for [M+H]⁺(C₂₄H₂₂Cl₂N₄O₂) requires m/z, 469.1/471.1, LCMS found m/z, 469.0/471.0; ¹H NMR (400 MHz, CHLOROFORM-d) δ−=8.38 (d, J=1.98 Hz, 1H) 7.57 (dd, J=8.93, 2.32 Hz, 1H) 7.36-7.45 (m, 2H) 7.27 (s, 2H) 6.56 (d, J=9.04 Hz, 1H) 4.36 (s, 2H) 3.66-3.76 (m, 2H) 3.53 (dt, J=7.11, 3.61 Hz, 1H) 3.35-3.42 (m, 2H) 2.10-2.25 (m, 1H) 1.65-1.79 (m, 2H) 1.44-1.53 (m, 2H) 1.23-1.33 (m, 2H) 1.07-1.17 (m, 2H).

(Z)-6-(4-((5-cyclopropyl-3-(2,6-dichlorophenyl)isoxazol-4-yl)methoxy)piperidin-1-yl)-N′-hydroxynicotinimidamide (6c). To a solution of 6-(4-((5-cyclopropyl-3-(2,6-dichlorophenyl)isoxazol-4-yl)methoxy)piperidin-1-yl)nicotinonitrile (6b) (120 mg, 255.67 umol) in ethanol (2 mL) was added hydroxylamine hydrochloride (35.53 mg, 511.33 umol) and TEA (51.74 mg, 511.33 umol, 71.17 uL) at 25° C. under N₂. The mixture was degassed and purged with N₂ 3 times and stirred at 85° C. for 16 hours under N₂ atmosphere. The reaction mixture was poured into H₂O (10 mL) and extracted with ethyl acetate (20 mL*2). The combined organic layers were washed with brine (10 mL), dried over anhydrous Na₂SO₄, filtered and concentrated under reduced pressure to give a residue which was purified by column chromatography to give 6c. MS mass calculated for [M+H]⁺(C₂₄H₂₅Cl₂N₅O₃) requires m/z, 502.1/504.1, LCMS found m/z, 502.0/504.0; ¹H NMR (400 MHz, CHLOROFORM-d) δ=8.38 (d, J=2.32 Hz, 1H) 7.74 (dd, J=5.69, 3.36 Hz, 1H) 7.38-7.43 (m, 2H) 7.29-7.35 (m, 1H) 6.59 (d, J=9.05 Hz, 1H) 4.77 (br s, 2H) 4.35 (s, 2H), 3.70-3.79 (m, 2H) 3.45-3.54 (m, 1H) 3.18-3.29 (m, 2H) 2.12-2.21 (m, 1H) 1.70-1.79 (m, 2H) 1.48 (dtd, J=12.55, 8.31, 8.31, 3.73 Hz, 2H) 1.24-1.31 (m, 2H) 1.09-1.17 (m, 2H).

3-(6-(4-((5-cyclopropyl-3-(2,6-dichlorophenyl)isoxazol-4-yl)methoxy)piperidin-1-yl)pyridin-3-yl)-1,2,4-oxadiazol-5(4H)-one (Compound 6). To a solution of (Z)-6-(4-((5-cyclopropyl-3-(2,6-dichlorophenyl)isoxazol-4-yl)methoxy)piperidin-1-yl)-N′-hydroxynicotinimidamide (6c) (90 mg, 179.14 umol) in diethyl carbonate (1 mL) and ethanol (1 mL) was added CH₃ONa (193.56 mg, 1.07 mmol, 30% in MeOH) at 25° C. in a sealed tube. The mixture was stirred at 100° C. for 16 hours and was poured into H₂O (10 mL) and extracted with ethyl acetate (20 mL*2). The combined organic layers were washed with brine (10 mL), dried over anhydrous Na₂SO₄, filtered and concentrated under reduced pressure to give a residue. The residue was purified by prep-TLC to give Compound 6. MS mass calculated for [M+H]⁺(C₂₅H₂₃Cl₂N₅O₄) requires m/z, 528.1/530.1, LCMS found m/z, 528.1/530.2; ¹H NMR (400 MHz, CHLOROFORM-d) δ=8.48 (d, J=2.20 Hz, 1H) 7.77 (dd, J=9.15, 2.32 Hz, 1H) 7.39-7.44 (m, 2H) 7.31-7.36 (m, 1H) 6.66 (d, J=9.04 Hz, 1H) 4.36 (s, 2H) 3.70-3.78 (m, 2H) 3.53 (brd, J=3.31 Hz, 1H) 3.35-3.43 (m, 2H) 2.11-2.21 (m, 1H) 1.69-1.79 (m, 2H) 1.51 (br s, 2H) 1.27 (td, J=7.00, 3.64 Hz, 2H) 1.11-1.17 (m, 2H).

### Example 7

### 3-(3-(4-((5-cyclopropyl-3-(2,6-dichlorophenyl)isoxazol-4-yl)methoxy)piperidin-1-yl)phenyl)-1,2,4-oxadiazol-5(4H)-one

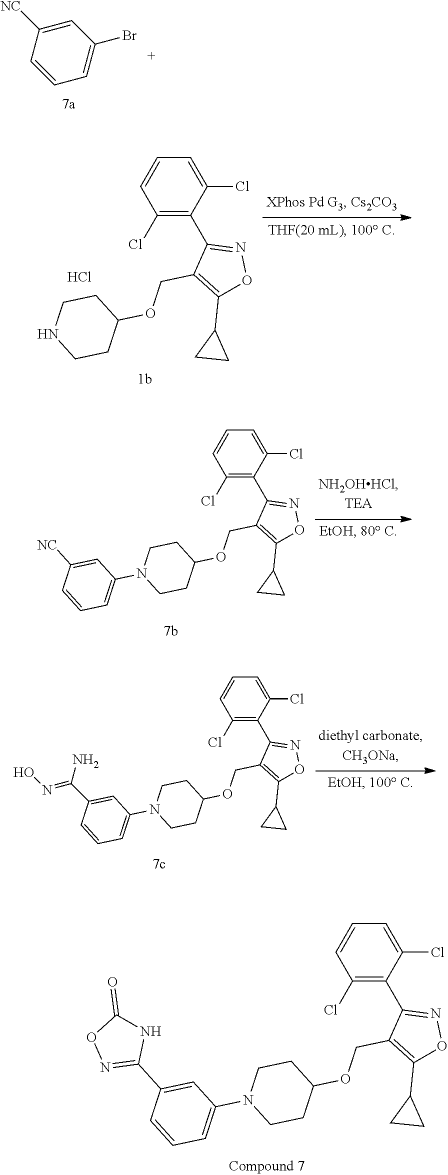

3-(4-((5-cyclopropyl-3-(2,6-dichlorophenyl)isoxazol-4-yl)methoxy)piperidin-1-yl)benzonitrile (7b). To a mixture of 3-bromobenzonitrile (7a) (135.25 mg, 743.07 umol) and 5-cyclopropyl-3-(2,6-dichlorophenyl)-4-((piperidin-4-yloxy)methyl)isoxazole hydrochloride (1b) (200 mg, 495.38 umol) in THE (20 mL) was added Cs₂CO₃ (322.81 mg, 990.76 umol), Xphos-Pd-G3 (50.32 mg, 59.45 umol) at 25° C. under N₂. The mixture was stirred at 100° C. for 16 hours and was poured into water (20 mL) and the mixture was extracted with ethyl acetate (20 mL×2). The combined organic layers were washed with brine (10 mL), dried with anhydrous Na₂SO₄, filtered and the filtrate was concentrated under reduced pressure. The residue was purified by prep-TLC to give 7b. MS mass calculated for [M+H]⁺(C₂₅H₂₃Cl₂N₃O₂) requires m/z, 468.1/470.1, LCMS found m/z, 468.1/469.9; ¹H NMR (400 MHz, CHLOROFORM-d) δ=7.41-7.34 (m, 2H), 7.29 (dd, J=6.6, 8.4 Hz, 2H), 7.08-7.01 (m, 3H), 4.33 (s, 2H), 3.43 (td, J=3.8, 7.4 Hz, 1H), 3.23 (ddd, J=3.6, 7.7, 11.6 Hz, 2H), 2.91 (ddd, J=3.6, 8.6, 12.2 Hz, 2H), 2.19-2.09 (m, 1H), 1.81-1.72 (m, 2H), 1.59 (m, 2H), 1.30-1.22 (m, 2H), 1.16-1.09 (m, 2H).

(Z)-3-(4-((5-cyclopropyl-3-(2,6-dichlorophenyl)isoxazol-4-yl)methoxy)piperidin-1-yl)-N′-hydroxybenzimidamide (7c). To a mixture of 3-(4-((5-cyclopropyl-3-(2,6-dichlorophenyl)isoxazol-4-yl)methoxy)piperidin-1-yl)benzonitrile (7b) (180 mg, 384.31 umol) and hydroxylamine hydrochloride (53.41 mg, 768.62 umol) in ethanol (20 mL) was added TEA (77.78 mg, 768.62 umol, 106.98 uL) at 25° C. under N₂. The mixture was stirred at 80° C. for 12 hours then was cooled to 25° C. and concentrated under reduced pressure. The residue was purified by prep-TLC to give 7c. MS mass calculated for [M+H]⁺(C₂₅H₂₆Cl₂N₄O₃) requires m/z, 501.1, 503.1 LCMS found m/z, 501.0, 503.0;

3-(3-(4-((5-cyclopropyl-3-(2,6-dichlorophenyl)isoxazol-4-yl)methoxy)piperidin-1-yl)phenyl)-1,2,4-oxadiazol-5(4H)-one (Compound 7). To a mixture of (Z)-3-(4-((5-cyclopropyl-3-(2,6-dichlorophenyl)isoxazol-4-yl)methoxy)piperidin-1-yl)-N′-hydroxybenzimidamide (7c) (120 mg, 239.33 umol) and diethyl carbonate (1.95 g, 16.51 mmol, 2 mL) in ethanol (2 mL) was added CH₃ONa (258.59 mg, 1.44 mmol, 30% in MeOH) at 25° C. in a sealed tube. The mixture was stirred at 100° C. for 12 hours and was concentrated under reduced pressure. The residue was purified by prep-TLC to give Compound 7. MS mass calculated for [M+H]⁺(C₂₆H₂₄Cl₂N₄O₄) requires m/z, 527.1, 529.1, LCMS found m/z, 527.2, 529.2; ¹H NMR (400 MHz, CHLOROFORM-d) δ=7.37-7.42 (m, 2H) 7.28-7.34 (m, 2H) 7.23 (s, 1H) 7.10 (br d, J=7.50 Hz, 1H) 7.03 (brd, J=7.50 Hz, 1H) 4.35 (s, 2H) 3.40-3.47 (m, 1H) 3.30 (br d, J=4.41 Hz, 2H) 2.94 (br t, J=8.93 Hz, 2H) 2.12-2.20 (m, 1H) 1.75-1.83 (m, 2H) 1.57 (dt, J=8.21, 4.38 Hz, 2H) 1.27 (br d, J=4.85 Hz, 2H) 1.10-1.17 (m, 2H).

### Example 8

### 3-(5-(4-((5-cyclopropyl-3-(2,6-dichlorophenyl)isoxazol-4-yl)methoxy)piperidin-1-yl)pyrimidin-2-yl)-1,2,4-oxadiazol-5(4H)-one

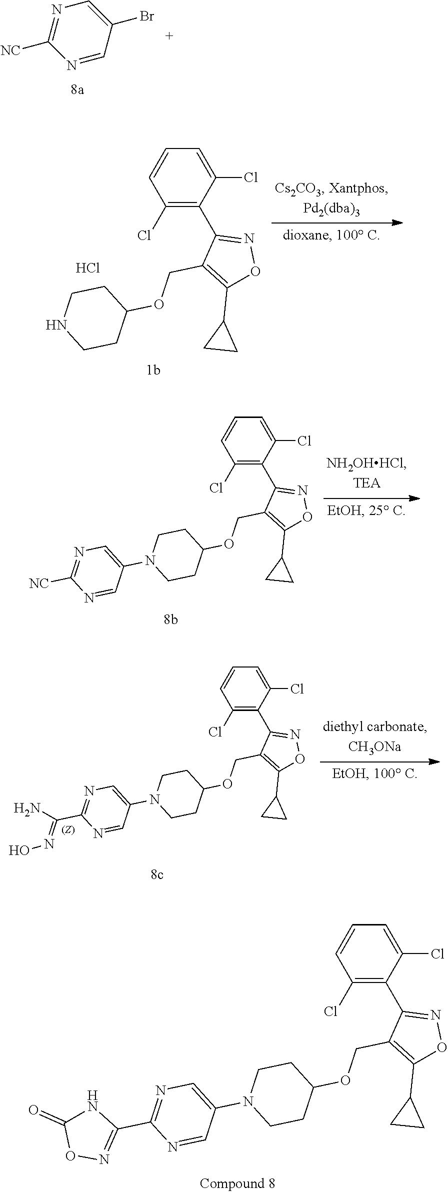

5-(4-((5-cyclopropyl-3-(2,6-dichlorophenyl)isoxazol-4-yl)methoxy)piperidin-1-yl)pyrimidine-2-carbonitrile (8b). To a solution of 5-bromopyrimidine-2-carbonitrile (8a) (105.21 mg, 571.79 umol) and 5-cyclopropyl-3-(2,6-dichlorophenyl)-4-((piperidin-4-yloxy)methyl)isoxazole hydrochloride (1b) (140 mg, 381.19 umol) in 1,4-dioxane (5 mL) was added Cs₂CO₃ (372.60 mg, 1.14 mmol), Xantphos (33.08 mg, 57.18 umol) and Pd₂(dba)₃ (17.45 mg, 19.06 umol) at 25° C. The reaction was degassed and purged with N₂ 3 times and heated to 100° C. for 16 hours. The reaction mixture was concentrated under reduced pressure to remove solvent. The residue was diluted with ethyl acetate (5 mL) and water (5 mL) and separated. The aqueous phase was extracted with ethyl acetate (5 mL*4). The combined organic layers were washed with brine (20 mL*2), dried with anhydrous Na₂SO₄, filtered and the filtrate was concentrated under reduced pressure. The residue was purified by prep-TLC (SiO₂, petroleum ether:ethyl acetate=2:1) to give 8b. ¹H NMR (400 MHz, CHLOROFORM-d) δ=8.46 (s, 2H), 7.45-7.40 (m, 2H), 7.37-7.31 (m, 1H), 4.36 (s, 2H), 3.93-3.85 (m, 2H), 3.69-3.61 (m, 2H), 3.58-3.51 (m, 1H), 2.19-2.11 (m, 1H), 1.73-1.66 (m, 2H), 1.53-1.45 (m, 2H), 1.28 (dd, J=2.3, 4.7 Hz, 2H), 1.17-1.11 (m, 2H).

(Z)-5-(4-((5-cyclopropyl-3-(2,6-dichlorophenyl)isoxazol-4-yl)methoxy)piperidin-1-yl)-N′-hydroxypyrimidine-2-carboximidamide (8c). To a solution of 5-(4-((5-cyclopropyl-3-(2,6-dichlorophenyl)isoxazol-4-yl)methoxy)piperidin-1-yl)pyrimidine-2-carbonitrile (8b) (80 mg, 170.09 umol) in ethanol (10 mL) was added hydroxylamine hydrochloride (23.64 mg, 340.17 umol) and TEA (34.42 mg, 340.17 umol, 47.35 uL) at 25° C. The resulting mixture was degassed and purged with N₂ 3 times, then stirred at 25° C. for 16 hours under N₂ atmosphere. The reaction mixture was concentrated under reduced pressure to remove solvent. The residue was purified by prep-TLC (SiO₂, ethyl acetate) to give 8c. ¹H NMR (400 MHz, CHLOROFORM-d) δ=8.50 (s, 2H), 7.43 (d, J=1.2 Hz, 1H), 7.41 (s, 1H), 7.35-7.31 (m, 1H), 4.73 (br s, 2H), 4.36 (s, 2H), 4.04-3.96 (m, 2H), 3.54-3.50 (m, 1H), 3.49-3.43 (m, 2H), 2.17 (tt, J=5.1, 8.5 Hz, 1H), 1.75-1.67 (m, 2H), 1.50-1.41 (m, 2H), 1.28 (dd, J=2.4, 5.0 Hz, 2H), 1.16-1.11 (m, 2H).

3-(5-(4-((5-cyclopropyl-3-(2,6-dichlorophenyl)isoxazol-4-yl)methoxy)piperidin-1-yl)pyrimidin-2-yl)-1,2,4-oxadiazol-5(4H)-one (Compound 8). To a mixture of (Z)-5-(4-((5-cyclopropyl-3-(2,6-dichlorophenyl)isoxazol-4-yl)methoxy)piperidin-1-yl)-N′-hydroxypyrimidine-2-carboximidamide (8c) (60 mg, 119.19 umol) in ethanol (3 mL) was added diethyl carbonate (1.46 g, 12.38 mmol, 1.5 mL) and CH₃ONa (128.79 mg, 715.17 umol, 30% in MeOH) at 25° C. in a sealed tube. The resulting mixture was degassed and purged with N₂ 3 times, and then stirred at 100° C. for 16 hours under N₂ atmosphere. The reaction mixture was concentrated under reduced pressure to remove solvent. The residue was diluted with ethyl acetate (5 mL) and water (5 mL) and the phases were separated. The aqueous phase was extracted with ethyl acetate (5 mL*4). The combined organic layer was washed with brine (20 mL*2), dried with anhydrous Na₂SO₄, filtered and concentrated under reduced pressure and purified by prep-HPLC (neutral condition: column: Waters Xbridge BEH C18 100*30 mm*10 um; mobile phase: [water(10 mM NH₄HCO₃)-ACN]; B %: 25%-50%, 8 min) to give Compound 8. MS mass calculated for [M+H]⁺(C₂₄H₂₂Cl₂N₆O₄) requires m/z, 529.1/531.1, LCMS found m/z 529.1/531.1; ¹H NMR (400 MHz, CHLOROFORM-d) δ=8.61 (s, 2H), 7.45-7.41 (m, 2H), 7.38-7.32 (m, 1H), 4.37 (s, 2H), 4.00-3.92 (m, 2H), 3.67-3.59 (m, 2H), 3.55 (td, J=3.6, 6.8 Hz, 1H), 2.21-2.13 (m, 1H), 1.77-1.67 (m, 2H), 1.55-1.46 (m, 2H), 1.32-1.26 (m, 2H), 1.18-1.12 (m, 2H).

### Example 9

### 3-(5-(4-((5-cyclopropyl-3-(2,6-dichlorophenyl)isoxazol-4-yl)methoxy)piperidin-1-yl)pyridin-2-yl)-1,2,4-oxadiazol-5(4H)-one

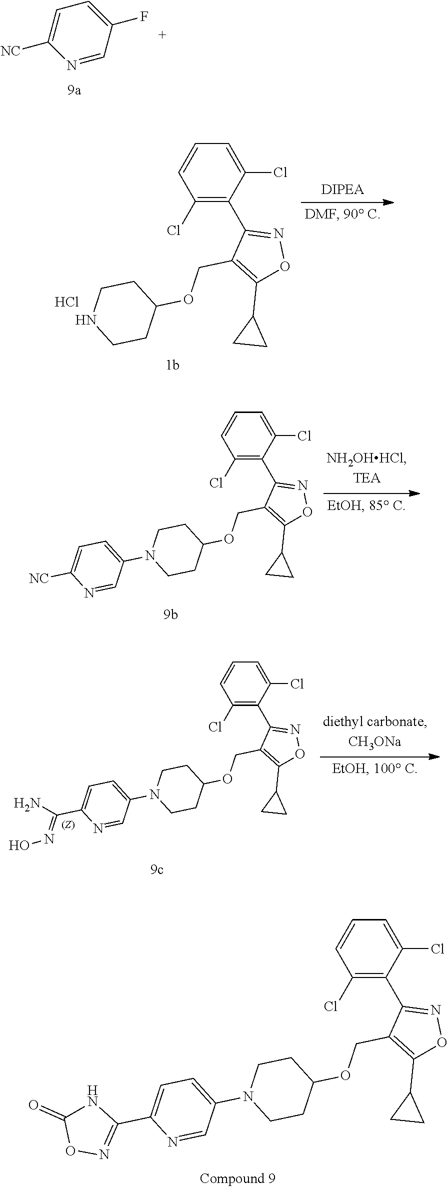

5-(4-((5-cyclopropyl-3-(2,6-dichlorophenyl)isoxazol-4-yl)methoxy)piperidin-1-yl)picolinonitrile (9b). To a solution of 5-cyclopropyl-3-(2,6-dichlorophenyl)-4-((piperidin-4-yloxy)methyl)isoxazole hydrochloride (1b) (100 mg, 247.69 umol) in DMF (2 mL) was added 5-fluoropicolinonitrile (9a) (36.29 mg, 297.23 umol) and DIPEA (64.02 mg, 495.37 umol) at 25° C. The mixture was degassed and purged with N₂ 3 times and heated to 90° C. for 18 hours. The reaction mixture was diluted with water (10 mL) and was extracted with ethyl acetate (5 mL*3). The combined organic layer was washed with brine (5 mL), dried over anhydrous Na₂SO₄, filtered and concentrated under reduced pressure to give a residue. The residue was purified by prep-TLC (SiO₂, petroleum ether:ethyl acetate=1:1) to give 9b. MS mass calculated for [M+H]⁺(C₂₄H₂₂Cl₂N₄O₂) requires m/z, 469.1/471.1, LCMS found m/z, 469.1/470.9; ¹H NMR (400 MHz, CHLOROFORM-d) δ=8.24 (d, J=2.9 Hz, 1H), 7.53-7.45 (m, 1H), 7.43-7.37 (m, 2H), 7.35-7.28 (m, 1H), 7.02 (dd, J=2.9, 8.8 Hz, 1H), 4.35 (s, 2H), 3.53 (td, J=3.2, 6.7 Hz, 1H), 3.33 (ddd, J=3.7, 8.3, 12.5 Hz, 2H), 3.21-3.10 (m, 2H), 2.21-2.08 (m, 1H), 1.77 (dt, J=4.2, 8.7 Hz, 2H), 1.64-1.56 (m, 2H), 1.34-1.25 (m, 2H), 1.17-1.08 (m, 2H).

(Z)-5-(4-((5-cyclopropyl-3-(2,6-dichlorophenyl)isoxazol-4-yl)methoxy)piperidin-1-yl)-N′hydroxypicolini-midamide (9c). To a solution of methyl 5-(4-((5-cyclopropyl-3-(2,6-dichlorophenyl)isoxazol-4-yl)methoxy)piperidin-1-yl)picolinonitrile (9b) (94 mg, 200.27 umol) in ethanol (5 mL) was added hydroxylamine hydrochloride (27.83 mg, 400.54 umol) and TEA (40.53 mg, 400.54 umol) at 25° C. and the mixture was heated to 85° C. for 16 hours. The reaction mixture was diluted with H₂O (10 mL) and extracted with ethyl acetate (10 mL*2). The combined organic layer was washed with brine (5 mL), dried over anhydrous Na₂SO₄, filtered and concentrated under reduced pressure to give a residue. The residue was purified by prep-TLC (SiO₂, dichloromethane:methanol=10:1) to give 9c. MS mass calculated for [M+H]⁺(C₂₄H₂₅Cl₂N₅O₃) requires m/z, 502.1/504.1, LCMS found m/z, 502.0/504.0.

3-(5-(4-((5-cyclopropyl-3-(2,6-dichlorophenyl)isoxazol-4-yl)methoxy)piperidin-1-yl)pyridin-2-yl)-1,2,4-oxadiazol-5(4H)-one (Compound 9). To a solution of (Z)-5-(4-((5-cyclopropyl-3-(2,6-dichlorophenyl)isoxazol-4-yl)methoxy)piperidin-1-yl)-N′-hydroxypicolinimidamide (9c) (80 mg, 159.24 umol) in ethanol (2 mL) was added CH₃ONa (172.05 mg, 955.43 umol, 30% in MeOH) and diethyl carbonate (1.95 g, 16.51 mmol, 2 mL) at 25° C. and the mixture was heated to 100° C. for 40 hours. The reaction mixture was poured into H₂O (5 mL) and extracted with ethyl acetate (10 mL*2). The combined organic layer was washed with brine (5 mL), dried over anhydrous Na₂SO₄, filtered and concentrated under reduced pressure to give a residue. The residue was purified by prep-TLC (SiO₂, dichloromethane:methanol=10:1) to give Compound 9. MS mass calculated for [M+H]⁺(C₂₅H₂₃Cl₂N₅O₄) requires m/z, 528.1/530.1, LCMS found m/z, 528.1/530.1; ¹H NMR (400 MHz, DMSO-d6) δ=9.11 (s, 1H), 7.83 (br d, J=9.3 Hz, 1H), 7.58 (s, 1H), 7.56 (s, 1H), 7.56-7.46 (m, 2H), 4.34 (s, 2H), 3.50 (br s, 1H), 3.42 (br s, 2H), 3.16 (br s, 2H), 2.35-2.29 (m, 1H), 1.74 (br s, 2H), 1.43 (br s, 2H), 1.15 (br d, J=8.2 Hz, 2H), 1.10 (br d, J=2.4 Hz, 2H); ¹H NMR (400 MHz, CHLOROFORM-d) δ=8.44-8.34 (m, 1H), 8.22 (br d, J=9.0 Hz, 1H), 7.71 (br d, J=8.7 Hz, 1H), 7.50-7.42 (m, 2H), 7.40 (br d, J=6.7 Hz, 1H), 4.44 (s, 2H), 3.69 (br s, 1H), 3.54 (br s, 2H), 3.43 (br s, 2H), 2.16 (br d, J=4.8 Hz, 1H), 1.88 (br s, 2H), 1.75 (br s, 2H), 1.32 (br s, 2H), 1.24-1.18 (m, 2H).

### Example 10

### 6-(4-(4-((5-cyclopropyl-3-(2,6-dichlorophenyl)isoxazol-4-yl)methoxy)piperidin-1-yl)phenyl)-1,2,4-triazine-3,5(2H,4H)-dione

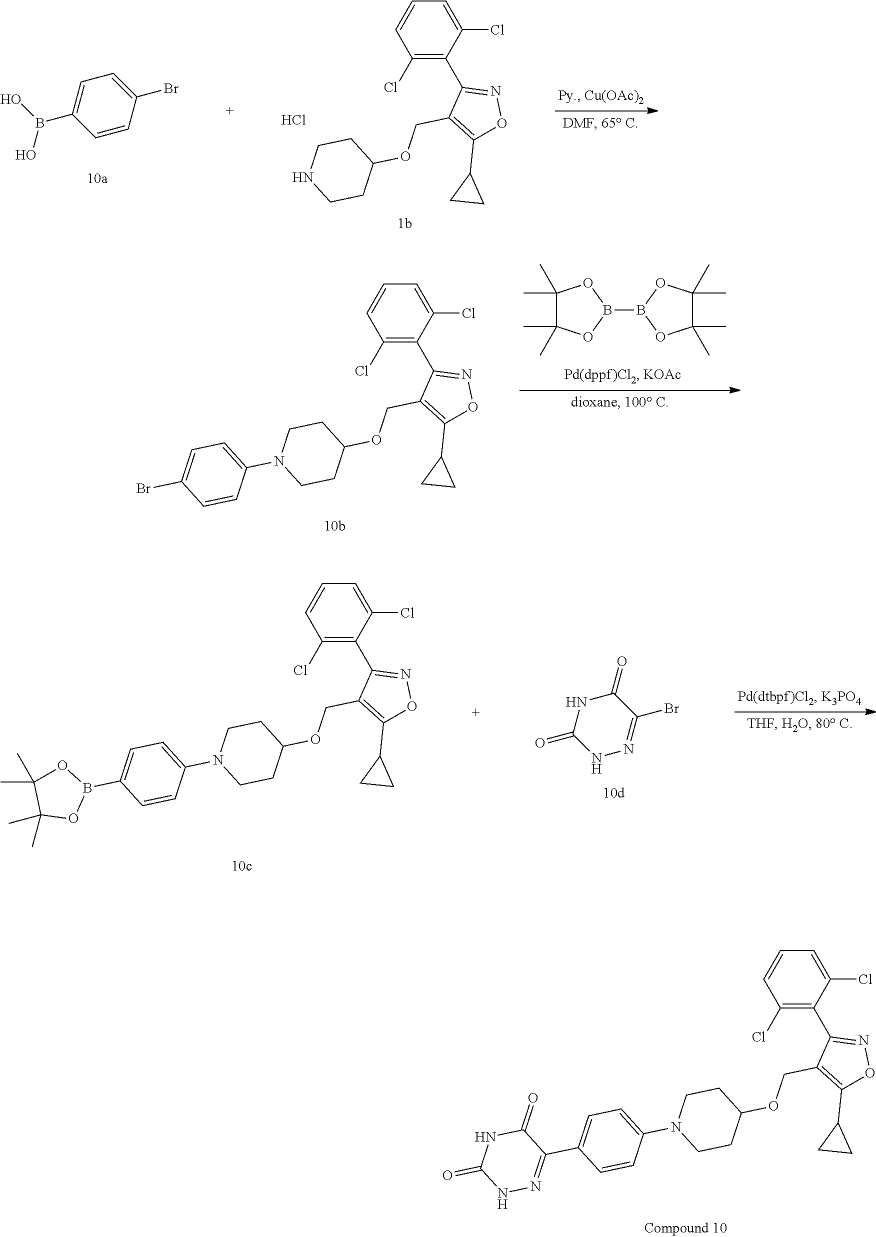

4-(((1-(4-bromophenyl) piperidin-4-yl) oxy) methyl)-5-cyclopropyl-3-(2, 6-dichlorophenyl)isoxazole (10b). To a solution of 5-cyclopropyl-3-(2,6-dichlorophenyl)-4-((piperidin-4-yloxy)methyl)isoxazole hydrochloride (1b) (280 mg, 762.38 umol) in DMF (5 mL) was added (4-bromophenyl)boronic acid (10a) (306.21 mg, 1.52 mmol), Cu(OAc)₂ (166.17 mg, 914.86 umol), pyridine (120.61 mg, 1.52 mmol) and 4 A M.S. at 25° C. and the mixture was heated to 65° C. for 24 hours under O₂ atmosphere. The reaction mixture was poured into H₂O (10 mL) and extracted with ethyl acetate (20 mL*3). The combined organic layer was washed with brine (10 mL), dried over anhydrous Na₂SO₄, filtered and concentrated under reduced pressure to give a residue. The residue was purified by prep-TLC (SiO₂, petroleum ether:ethyl acetate=3:1) to give 10b. MS mass calculated for [M+H]⁺(C₂₄H₂₃BrCl₂N₂O₂) requires m/z, 521.0, LCMS found m/z, 521.0; ¹H NMR (400 MHz, CHLOROFORM-d) δ=7.42-7.36 (m, 2H), 7.34-7.28 (m, 3H), 6.77-6.71 (m, 2H), 4.34 (s, 2H), 3.40 (tt, J=3.7, 7.8 Hz, 1H), 3.25-3.18 (m, 2H), 2.84 (ddd, J=3.4, 8.7, 12.3 Hz, 2H), 2.16 (tt, J=5.1, 8.4 Hz, 1H), 1.83-1.74 (m, 2H), 1.58-1.53 (m, 2H), 1.28 (dd, J=2.4, 4.9 Hz, 2H), 1.16-1.10 (m, 2H).

5-cyclopropyl-3-(2,6-dichlorophenyl)-4-(((1-(4-(4,4,5,5-tetramethyl-1,3,2-dioxaborolan-2-yl)phenyl)piperidin-4-yl)oxy)methyl)isoxazole (10c). To a solution of methyl 4-(((1-(4-bromophenyl)piperidin-4-yl)oxy)methyl)-5-cyclopropyl-3-(2,6-dichlorophenyl)isoxazole (10b) (70 mg, 134.03 umol) in 1,4-dioxane (4 mL) was added 4,4,5,5-tetramethyl-2-(4,4,5,5-tetramethyl-1,3,2-dioxaborolan-2-yl)-1,3,2-dioxaborolane (102.11 mg, 402.10 umol), Pd(dppf)Cl₂ (9.81 mg, 13.40 umol) and KOAc (26.31 mg, 268.07 umol) at 25° C. and the mixture was heated to 100° C. for 16 hours. The reaction mixture was poured into H₂O (10 mL) and extracted with ethyl acetate (20 mL*2). The combined organic layer was washed with brine (10 mL), dried over anhydrous Na₂SO₄, filtered and concentrated under reduced pressure to give a residue. The residue was purified by prep-TLC (SiO₂, petroleum ether:ethyl acetate=3:1) to give 10c. MS mass calculated for [M+H]⁺(C₃₀H₃₅BCl₂N₂O₄) requires m/z, 569.2/571.2, LCMS found m/z, 569.2/571.2.

6-(4-(4-((5-cyclopropyl-3-(2,6-dichlorophenyl)isoxazol-4-yl)methoxy)piperidin-1-yl)phenyl)-1,2,4-triazine-3,5(2H,4H)-dione (Compound 10). To a solution of 5-cyclopropyl-3-(2,6-dichlorophenyl)-4-(((1-(4-(4,4,5,5-tetramethyl-1,3,2-dioxaborolan-2-yl)phenyl)piperidin-4-yl)oxy)methyl)isoxazole (10c) (40 mg, 70.26 umol) and 6-bromo-1,2,4-triazine-3,5(2H,4H)-dione (10d) (40.46 mg, 210.78 umol) in THF (4 mL) and H₂O (1 mL) was added K₃PO₄ (29.83 mg, 140.52 umol) and Pd(dtbpf)Cl₂ (4.58 mg, 7.03 umol) at 25° C. and the mixture was heated to 80° C. for 16 hours. The reaction mixture was poured into H₂O (10 mL) and extracted with ethyl acetate (20 mL*2). The combined organic layer was washed with brine (10 mL), dried over anhydrous Na₂SO₄, filtered and concentrated under reduced pressure to give a residue. The residue was purified by prep-HPLC (column: Phenomenex Luna C18 150*30 mm*5 um; mobile phase: [water (0.10% TFA)-ACN]; B %: 40%-70%, 10 min) to give Compound 10. MS mass calculated for [M+H]⁺(C₂₇H₂₅Cl₂N₅O₄) requires m/z, 554.1, LCMS found m/z, 554.1/556.1; ¹H NMR (400 MHz, CHLOROFORM-d) δ=10.35 (br s, 1H), 9.53 (br s, 1H), 8.04 (br d, J=7.0 Hz, 2H), 7.46-7.36 (m, 2H), 7.31 (br d, J=8.2 Hz, 3H), 4.36 (s, 2H), 3.61 (br s, 1H), 3.35 (br d, J=9.5 Hz, 2H), 3.26 (br s, 2H), 2.19-2.02 (m, 3H), 1.77 (br s, 2H), 1.33-1.25 (m, 2H), 1.20-1.10 (m, 2H).

### Example 11

### 3-(4-(4-((5-cyclopropyl-3-(2,6-dichlorophenyl)isoxazol-4-yl)methoxy)piperidin-1-yl)-2-methylphenyl)-1,2,4-oxadiazol-5(4H)-one

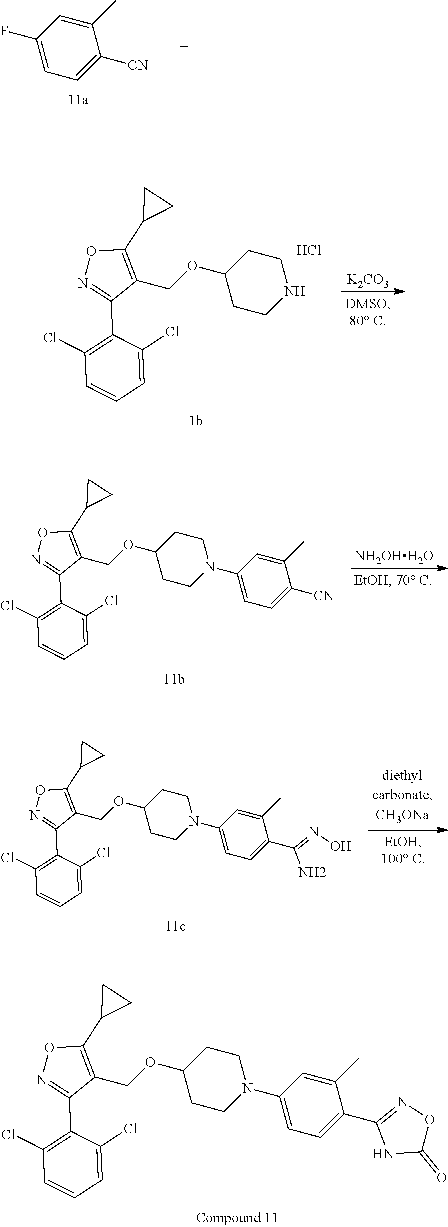

4-(4-((5-cyclopropyl-3-(2,6-dichlorophenyl)isoxazol-4-yl)methoxy)piperidin-1-yl)-2-methylbenzonitrile (11b). To a solution of 4-fluoro-2-methylbenzonitrile (11a) (100 mg, 739.98 umol) and 5-cyclopropyl-3-(2,6-dichlorophenyl)-4-((piperidin-4-yloxy)methyl)isoxazole hydrochloride (1b) (271.77 mg, 739.98 umol) in DMSO (3 mL) was added K₂CO₃ (204.55 mg, 1.48 mmol) at 25° C. Then the reaction was degassed and purged with N₂ 3 times and the mixture was stirred at 80° C. for 16 hours under N₂ atmosphere. The reaction mixture was concentrated under reduced pressure to remove solvent. The residue was diluted with ethyl acetate (5 mL) and water (5 mL) and extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, petroleum ether:ethyl acetate=3:1) to give 11b. MS mass calculated for [M+H]⁺(C₂₆H₂₅Cl₂N₃O₂) requires m/z, 482.1/484.1, LCMS found m/z 482.1/484.1; ¹H NMR (400 MHz, CHLOROFORM-d) δ=7.42-7.38 (m, 3H), 7.33-7.28 (m, 1H), 6.67-6.62 (m, 2H), 4.35 (s, 2H), 3.51-3.44 (m, 1H), 3.40-3.32 (m, 2H), 3.08-3.00 (m, 2H), 2.46 (s, 3H), 2.15 (tt, J=5.1, 8.4 Hz, 1H), 1.80-1.72 (m, 2H), 1.55-1.48 (m, 2H), 1.29-1.26 (m, 2H), 1.16-1.10 (m, 2H).

(Z)-4-(4-((5-cyclopropyl-3-(2,6-dichlorophenyl)isoxazol-4-yl)methoxy)piperidin-1-yl)-N′-hydroxy-2-methylbenzimidamide (tic). To a solution of 4-(4-((5-cyclopropyl-3-(2,6-dichlorophenyl)isoxazol-4-yl)methoxy)piperidin-1-yl)-2-methylbenzonitrile (11b) (50 mg, 103.65 umol) in ethanol (3 mL) was added hydroxylamine (20.54 mg, 310.94 umol, 3 mL, 50% in water) at 25° C., the reaction mixture was degassed and purged with N₂ 3 times, and then the mixture was stirred at 70° C. for 48 hours under N₂ atmosphere. The reaction mixture was concentrated under reduced pressure to remove solvent. The residue was diluted with ethyl acetate (5 mL) and water (5 mL), then extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, petroleum ether:ethyl acetate=1:1) to give 11c. ¹H NMR (400 MHz, CHLOROFORM-d) δ=7.43-7.38 (m, 2H), 7.34-7.28 (m, 1H), 7.27-7.23 (m, 1H), 6.72-6.66 (m, 2H), 4.74 (br s, 2H), 4.34 (s, 2H), 3.40 (qd, J=3.9, 7.8 Hz, 1H), 3.35-3.28 (m, 2H), 2.98-2.84 (m, 2H), 2.40 (s, 3H), 2.16 (tt, J=5.1, 8.4 Hz, 1H), 1.82-1.75 (m, 2H), 1.59-1.49 (m, 2H), 1.29-1.26 (m, 2H), 1.15-1.10 (m, 2H).

3-(4-(4-((5-cyclopropyl-3-(2,6-dichlorophenyl)isoxazol-4-yl)methoxy)piperidin-1-yl)-2-methylphenyl)-1,2,4-oxadiazol-5(4H)-one (Compound 11). A mixture of (Z)-4-(4-((5-cyclopropyl-3-(2,6-dichlorophenyl)isoxazol-4-yl)methoxy)piperidin-1-yl)-N′-hydroxy-2-methylbenzimidamide (11c) (60 mg, 116.41 umol) in ethanol (3 mL) was added diethyl carbonate (1.46 g, 12.38 mmol, 1.5 mL) and CH₃ONa (125.77 mg, 698.45 umol, 30% in MeOH) at 25° C. in a sealed tube. Then the reaction mixture was stirred at 100° C. for 48 hours. The reaction mixture was concentrated under reduced pressure to remove solvent. The residue was diluted with ethyl acetate (5 mL) and water (5 mL), then extracted with ethyl acetate (5 mL*4). The combined organic phase was washed with brine (20 mL*2), dried with anhydrous Na₂SO₄, filtered and concentrated under reduced pressure and purified by prep-HPLC (neutral condition: column: Waters Xbridge Prep OBD C18 150*40 mm*10 um; mobile phase: [water(10 mM NH₄HCO₃)-ACN]; B %: 25%-60%, 8 min) to give Compound 11. MS mass calculated for [M+H]⁺(C₂₇H₂₆Cl₂N₄O₄) requires m/z, 541.1/543.1, LCMS found m/z 541.1/543.1; ¹H NMR (400 MHz, CHLOROFORM-d) δ=7.43-7.38 (m, 2H), 7.38-7.29 (m, 2H), 6.78-6.71 (m, 2H), 4.36 (s, 2H), 3.47 (tt, J=3.6, 7.4 Hz, 1H), 3.43-3.35 (m, 2H), 3.08-3.00 (m, 2H), 2.53 (s, 3H), 2.16 (tt, J=5.1, 8.5 Hz, 1H), 1.82-1.73 (m, 2H), 1.55 (dtd, J=3.7, 8.2, 12.4 Hz, 2H), 1.31-1.26 (m, 2H), 1.17-1.11 (m, 2H).

### Example 12

### 5-(4-(4-((5-cyclopropyl-3-(2,6-dichlorophenyl)isoxazol-4-yl)methoxy)piperidin-1-yl)phenyl)-1,3,4-oxadiazol-2(3H)-one

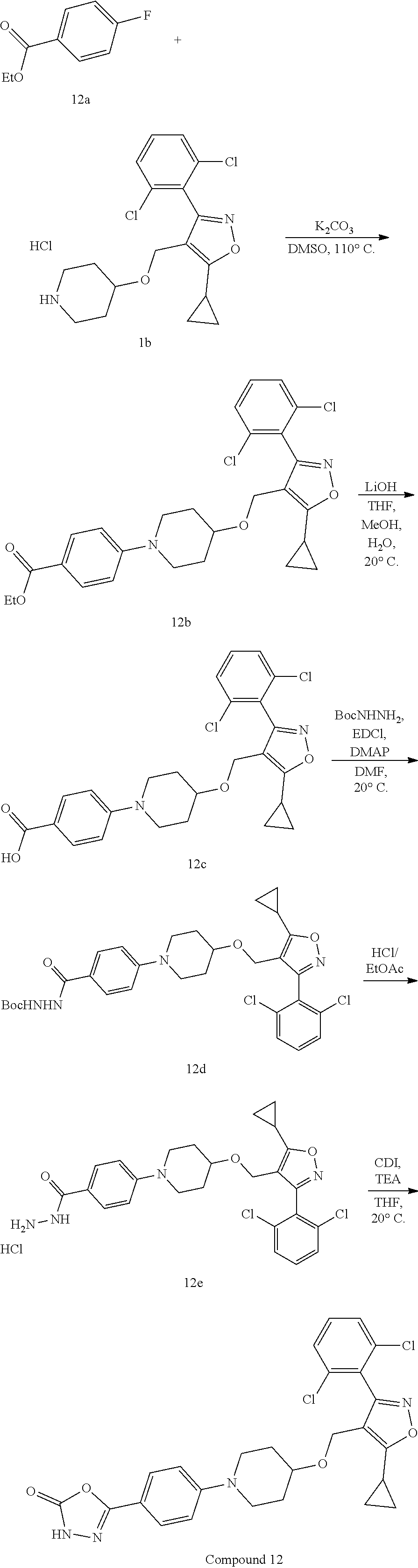

Ethyl 4-(4-((5-cyclopropyl-3-(2,6-dichlorophenyl)isoxazol-4-yl)methoxy)piperidin-1-yl)benzoate (12b). To a solution of ethyl 4-fluorobenzoate (12a) (187.44 mg, 1.11 mmol, 164.42 uL) and 5-cyclopropyl-3-(2,6-dichlorophenyl)-4-((piperidin-4-yloxy)methyl)isoxazole hydrochloride (1b) (300 mg, 743.07 umol) in DMSO (2 mL) was added K₂CO₃ (308.09 mg, 2.23 mmol) at 20° C., the mixture was then stirred at 110° C. for 16 hours. The reaction mixture was poured into water (10 mL) and extracted with ethyl acetate (15 ml*3), the combined organic layer was washed with brine (10 mL*2), dried over anhydrous Na₂SO₄ and filtered, the filtrate was concentrated and the residue was purified by silica gel column to give (petroleum ether:ethyl acetate=20:1 to 5:1) to give 12b: ¹H NMR (CHLOROFORM-d, 400 MHz): δ=7.90 (d, J=9.0 Hz, 2H), 7.36-7.42 (m, 2H), 7.27-7.32 (m, 1H), 6.81 (d, J=9.0 Hz, 2H), 4.28-4.38 (m, 4H), 3.47 (tt, J=7.5, 3.6 Hz, 1H), 3.32-3.41 (m, 2H), 3.03 (ddd, J=12.6, 8.6, 3.5 Hz, 2H), 2.12-2.20 (m, 1H), 1.72-1.84 (m, 2H), 1.48-1.57 (m, 2H), 1.37 (t, J=7.1 Hz, 3H), 1.27-1.29 (m, 2H), 1.10-1.17 (m, 2H).

4-(4-((5-cyclopropyl-3-(2,6-dichlorophenyl)isoxazol-4-yl)methoxy)piperidin-1-yl)benzoic acid (12c). To a solution of ethyl 4-(4-((5-cyclopropyl-3-(2,6-dichlorophenyl)isoxazol-4-yl)methoxy)piperidin-1-yl)benzoate (12b) (150 mg, 291.02 umol) in THF (0.13 mL), methanol (0.13 mL) and H₂O (0.13 mL) was added LiOH H₂O (0.9 M, 1.29 mL) at 20° C. The mixture was stirred at 20° C. for 12 hours. The reaction mixture was concentrated to remove organic solvents. The aqueous phase was adjusted to pH 4 with diluted HCl solution and the mixture was then extracted with ethyl acetate (10 mL*3). The combined organic layer was dried over anhydrous Na₂SO₄ and concentrated to give 12c (120 mg, crude) as red oil which was used directly to next step. MS mass calculated for [M+H]⁺(C₂₅H₂₄Cl₂N₂O₄) requires m/z, 487.1/489.1, LCMS found m/z, 487.1/489.1; ¹H NMR (CHLOROFORM-d, 400 MHz): δ=7.96 (d, J=8.9 Hz, 2H), 7.36-7.42 (m, 2H), 7.28-7.33 (m, 1H), 6.82 (d, J=9.0 Hz, 2H), 4.35 (s, 2H), 3.48 (tt, J=7.3, 3.5 Hz, 1H), 3.35-3.44 (m, 2H), 3.03-3.13 (m, 2H), 2.15 (tt, J=8.4, 5.1 Hz, 1H), 1.72-1.83 (m, 2H), 1.49-1.61 (m, 2H), 1.25-1.31 (m, 2H), 1.10-1.17 (m, 2H).

Tert-butyl 2-(4-(4-((5-cyclopropyl-3-(2,6-dichlorophenyl)isoxazol-4-yl)methoxy)piperidin-1-yl)benzoyl)-hydrazinecarboxylate (12d). To a solution of 4-(4-((5-cyclopropyl-3-(2,6-dichlorophenyl)isoxazol-4-yl)methoxy)piperidin-1-yl)benzoic acid (12c) (100 mg, 205.18 umol) and tert-butyl N-aminocarbamate (32.54 mg, 246.22 umol) in DMF (2 mL) was added EDCI (51.13 mg, 266.74 umol) and DMAP (501.33 ug, 4.10 umol) at 20° C. and the mixture was stirred at 20° C. for 2 hours. The reaction mixture was then poured into water (10 mL) and extracted with ethyl acetate (10 mL*3). The combined organic layers were washed with brine (10 mL), dried over anhydrous Na₂SO₄ and concentrated. The residue was purified by prep-TLC (petroleum ether:ethyl acetate=1:1) to give 12d. MS mass calculated for [M+H]⁺(C₃₀H₃₄Cl₂N₄O₅) requires m/z, 601.2/603.2, LCMS found m/z, 601.2/603.1; ¹H NMR (CHLOROFORM-d, 400 MHz): δ=7.81 (br s, 1H), 7.69 (d, J=8.9 Hz, 2H), 7.34-7.41 (m, 2H), 7.26-7.31 (m, 2H), 6.80 (d, J=8.9 Hz, 2H), 6.61-6.74 (m, 1H), 4.34 (s, 2H), 3.46 (tt, J=7.4, 3.6 Hz, 1H), 3.28-3.38 (m, 2H), 2.97-3.08 (m, 2H), 2.15 (tt, J=8.5, 5.1 Hz, 1H), 1.73-1.81 (m, 2H), 1.52-1.60 (m, 2H), 1.50 (s, 9H), 1.25-1.30 (m, 2H), 1.09-1.16 (m, 2H).

4-(4-((5-cyclopropyl-3-(2,6-dichlorophenyl)isoxazol-4-yl)methoxy)piperidin-1-yl)benzohydrazide (12e). Tert-butyl 2-(4-(4-((5-cyclopropyl-3-(2,6-dichlorophenyl)isoxazol-4-yl)methoxy)piperidin-1-yl)benzoyl)-hydrazinecarboxylate (12d) (110 mg, 182.87 umol) was dissolved in ethyl acetate (2 mL) and then HCl/Ethyl acetate (2 mL) was added. The mixture was stirred at 20° C. for 2 hours. The reaction mixture was concentrated under reduced pressure to give 12d; MS mass calculated for [M+H]⁺(C₂₅H₂₆Cl₂N₄O₃) requires m/z, 501.1/503.1, LCMS found m/z, 501.1/503.1.

5-(4-(4-((5-cyclopropyl-3-(2,6-dichlorophenyl)isoxazol-4-yl)methoxy)piperidin-1-yl)phenyl)-1,3,4-oxadiazol-2(3H)-one (Compound 12). To a solution of 4-(4-((5-cyclopropyl-3-(2,6-dichlorophenyl)isoxazol-4-yl)methoxy)piperidin-1-yl)benzo-hydrazide (12e) (100 mg, 185.92 umol, HCl) and TEA (56.44 mg, 557.76 umol, 77.63 uL) in THE (5 mL) was stirred for 2 min, then CDI (60.29 mg, 371.84 umol) was added at 20° C. The mixture was then stirred for 12 hours. The reaction mixture was quenched with water (10 mL) and extracted with ethyl acetate (10 mL*3). The combined organic layers were washed with brine (10 mL), dried over anhydrous Na₂SO₄, filtered, and the filtrate was concentrated. The residue was then purified by prep-TLC to give Compound 12; MS mass calculated for [M+H]⁺(C₂₆H₂₄Cl₂N₄O₄) requires m/z, 527.1/529.1, LCMS found m/z, 527.1/529.1; ¹H NMR (CHLOROFORM-d, 400 MHz): δ=8.39 (br s, 1H), 7.61 (d, J=9.0 Hz, 2H), 7.28-7.35 (m, 2H), 7.22 (dd, J=8.8, 7.3 Hz, 1H), 6.79 (d, J=9.0 Hz, 2H), 4.28 (s, 2H), 3.40 (tt, J=7.4, 3.6 Hz, 1H), 3.23-3.34 (m, 2H), 2.96 (ddd, J=12.6, 8.6, 3.5 Hz, 2H), 2.08 (tt, J=8.5, 5.1 Hz, 1H), 1.65-1.76 (m, 2H), 1.42-1.57 (m, 2H), 1.16-1.25 (m, 2H), 1.00-1.11 (m, 2H).

### Example 13

### 3-(4-(4-((5-cyclopropyl-3-(2,6-dichlorophenyl)isoxazol-4-yl)methoxy)piperidin-1-yl)-2-fluorophenyl)-1,2,4-oxadiazol-5(4H)-one

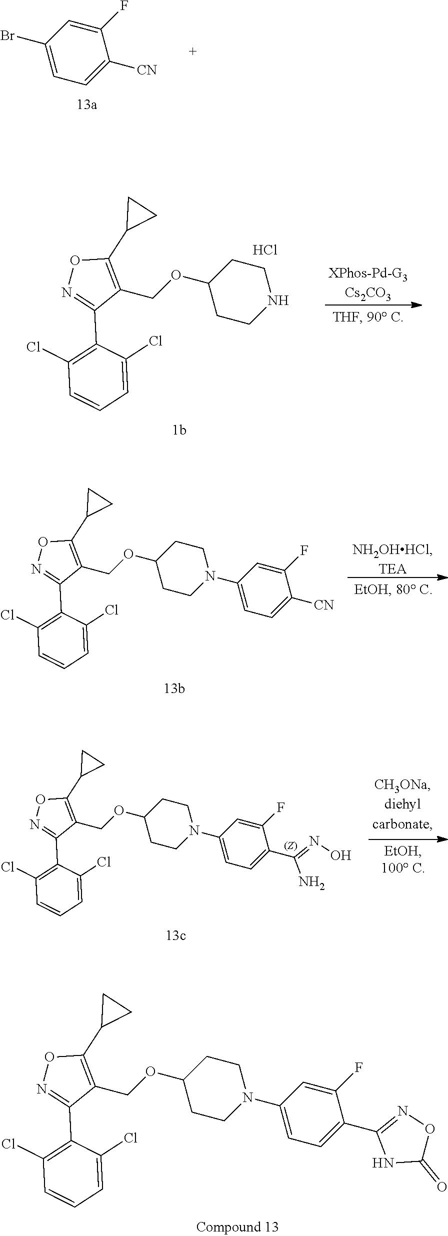

4-(4-((5-cyclopropyl-3-(2,6-dichlorophenyl)isoxazol-4-yl)methoxy)piperidin-1-yl)-2-fluorobenzonitrile (13b). To a solution of 4-bromo-2-fluorobenzonitrile (13a) (111.46 mg, 557.30 umol) and 5-cyclopropyl-3-(2,6-dichlorophenyl)-4-((piperidin-4-yloxy)methyl)isoxazole hydrochloride (1b) (150 mg, 371.54 umol) in THF (5 mL) was added Cs₂CO₃ (242.11 mg, 743.07 umol) and XPhos-Pd-G3 (37.74 mg, 44.58 umol) at 25° C. The mixture was degassed and purged with N₂ 3 times, then stirred at 90° C. for 10 hours. The reaction mixture was poured into H₂O (10 mL) and extracted with ethyl acetate (20 mL*2). The combined organic layer was washed with brine (10 mL), dried over anhydrous Na₂SO₄, filtered and concentrated under reduced pressure to give a residue. The residue was purified by prep-TLC to give 13b. MS mass calculated for [M+H]⁺(C₂₅H₂₂Cl₂FN₃O₂) requires m/z, 486.1/488.1, LCMS found m/z, 486.1/487.9; ¹H NMR (400 MHz, CHLOROFORM-d) δ=7.28-7.43 (m, 4H) 6.57 (dd, J=8.93, 2.32 Hz, 1H) 6.41-6.51 (m, 1H) 4.35 (s, 2H) 3.52 (dt, J=6.67, 3.61 Hz, 1H) 3.27-3.39 (m, 2H) 3.05-3.17 (m, 2H) 2.09-2.19 (m, 1H) 1.74 (td, J=8.54, 3.86 Hz, 2H) 1.48-1.63 (m, 2H) 1.22-1.32 (m, 2H) 1.09-1.18 (m, 2H).

(Z)-4-(4-((5-cyclopropyl-3-(2,6-dichlorophenyl)isoxazol-4-yl)methoxy)piperidin-1-yl)-2-fluoro-N′-hydroxybenzimidamide (13c). To a solution of 4-(4-((5-cyclopropyl-3-(2,6-dichlorophenyl)isoxazol-4-yl)methoxy)piperidin-1-yl)-2-fluorobenzonitrile (13b) (140 mg, 287.85 umol) in ethanol (5 mL) was added hydroxylamine hydrochloride (40.01 mg, 575.70 umol) and TEA (58.25 mg, 575.70 umol, 80.13 uL) at 25° C. The mixture was stirred at 80° C. for 16 hours. The reaction mixture was poured into H₂O (10 mL) and the mixture was extracted with ethyl acetate (20 mL*2). The combined organic layer was washed with brine (10 mL), dried over anhydrous Na₂SO₄, filtered and concentrated under reduced pressure to give a residue. The residue was purified by column chromatography to give 13c. MS mass calculated for [M+H]⁺(C₂₅H₂₅Cl₂FN₄O₃) requires m/z, 519.1/521.1, LCMS found m/z, 519.2/520.9; ¹H NMR (400 MHz, CHLOROFORM-d) δ=7.53 (t, J=8.93 Hz, 1H) 7.36-7.43 (m, 2H) 7.27-7.34 (m, 1H) 6.54-6.64 (m, 1H) 6.49 (dd, J=15.77, 2.54 Hz, 1H) 5.08 (br s, 2H) 4.29-4.37 (m, 2H) 3.46 (td, J=7.22, 3.20 Hz, 1H) 3.23-3.34 (m, 2H) 2.91-3.01 (m, 2H) 2.10-2.21 (m, 1H) 1.68-1.83 (m, 2H) 1.55 (br dd, J=8.38, 3.97 Hz, 2H) 1.26-1.31 (m, 2H) 1.06-1.18 (m, 2H).

3-(4-(4-((5-cyclopropyl-3-(2,6-dichlorophenyl)isoxazol-4-yl)methoxy)piperidin-1-yl)-2-fluorophenyl)-1,2,4-oxadiazol-5(4H)-one (Compound 13). A solution of (Z)-4-(4-((5-cyclopropyl-3-(2,6-dichlorophenyl)isoxazol-4-yl)methoxy)piperidin-1-yl)-2-fluoro-N′-hydroxybenzimidamide (13c) (50 mg, 96.27 umol) in ethanol (1.5 mL) and diethyl carbonate (1 mL) was added CH₃ONa (173.35 mg, 962.66 umol, 30% in MeOH) at 25° C. The mixture was degassed and purged with N₂ 3 times and stirred at 100° C. for 16 hours under N₂ atmosphere. The reaction mixture was dried in vacuum and the residue was purified by prep-HPLC to give Compound 13. MS mass calculated for [M+H]⁺(C₂₆H₂₃Cl₂FN₄O₄) requires m/z, 545.1/547.1, LCMS found m/z, 545.2/547.1; ¹H NMR (400 MHz, CHLOROFORM-d) δ=7.76 (t, J=8.93 Hz, 1H) 7.37-7.44 (m, 2H) 7.28-7.35 (m, 1H) 6.65-6.72 (m, 1H) 6.52 (br d, J=17.64 Hz, 1H) 4.33-4.38 (m, 1H) 4.35 (s, 1H) 3.52 (br d, J=3.53 Hz, 1H) 3.31-3.40 (m, 2H) 3.07-3.16 (m, 2H) 2.09-2.19 (m, 1H) 1.71-1.81 (m, 2H) 1.55 (br s, 2H) 1.25-1.32 (m, 2H) 1.10-1.17 (m, 2H).

### Example 14

### 3-(2-chloro-4-(4-((5-cyclopropyl-3-(2,6-dichlorophenyl)isoxazol-4-yl)methoxy)piperidin-1-yl)phenyl)-1,2,4-oxadiazol-5(4H)-one

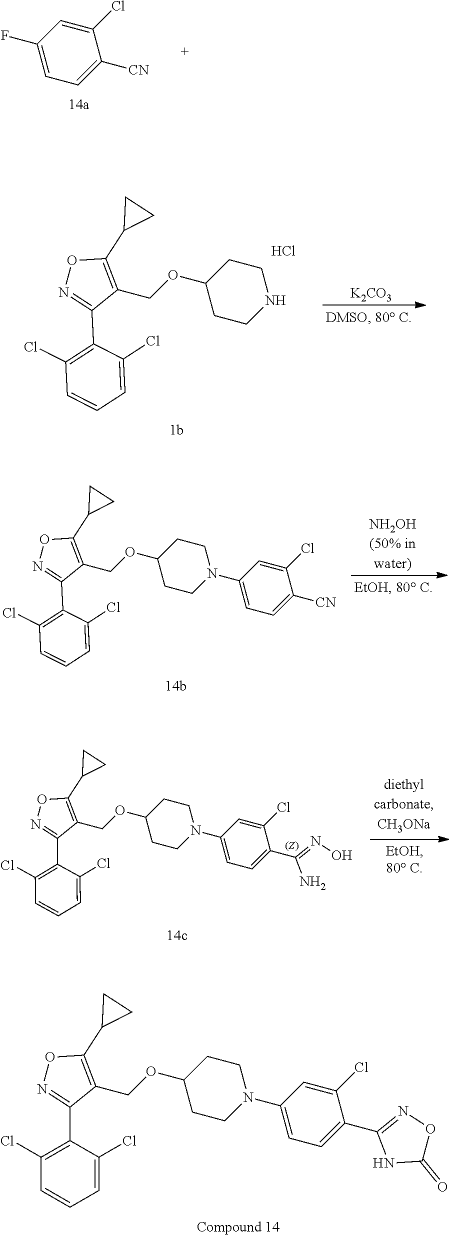

2-chloro-4-(4-((5-cyclopropyl-3-(2,6-dichlorophenyl)isoxazol-4-yl)methoxy)piperidin-1-yl)benzonitrile (14b). To a solution of 2-chloro-4-fluoro-benzonitrile (231.18 mg, 1.49 mmol) and 5-cyclopropyl-3-(2,6-dichlorophenyl)-4-((piperidin-4-yloxy)methyl)isoxazole hydrochloride (1b) (300 mg, 743.07 umol) in DMSO (1 mL) was added K₂CO₃ (308.09 mg, 2.23 mmol) in one portion. The reaction mixture was stirred for 12 hours at 80° C. and was poured into water (10 mL) and extracted with ethyl acetate (10 mL*2). The combined organic layer was washed with brine (10 mL*2), dried over anhydrous Na₂SO₄, filtered and concentrated. The residue was purified by silica gel column chromatography to give 14b. MS mass calculated for [M+H]⁺(C₂₅H₂₂Cl₃N₃O₂) requires m/z, 502.1/504.1 LCMS found m/z, 502.1/504.1; ¹H NMR (CHLOROFORM-d, 400 MHz): δ=7.37-7.45 (m, 3H), 7.28-7.34 (m, 1H), 6.80 (d, J=2.4 Hz, 1H), 6.67 (dd, J=8.9, 2.5 Hz, 1H), 4.35 (s, 2H), 3.51 (tt, J=6.9, 3.5 Hz, 1H), 3.26-3.36 (m, 2H), 3.03-3.16 (m, 2H), 2.14 (tt, J=8.5, 5.1 Hz, 1H), 1.67-1.79 (m, 2H), 1.50-1.60 (m, 2H), 1.25-1.31 (m, 2H), 1.10-1.17 (m, 2H).

(Z)-2-chloro-4-(4-((5-cyclopropyl-3-(2,6-dichlorophenyl)isoxazol-4-yl)methoxy)piperidin-1-yl)-N′-hydroxybenzimidamide (14c). To a solution of 2-chloro-4-(4-((5-cyclopropyl-3-(2,6-dichlorophenyl)isoxazol-4-yl)methoxy)piperidin-1-yl)benzonitrile (14b) (280 mg, 556.86 umol) in ethanol (10 mL) was added hydroxylamine (1.40 g, 10.07 mmol, 2 mL, 50% in water) at 20° C. The reaction mixture was heated to 80° C. and stirred for 12 hours. The reaction mixture was concentrated to remove the ethanol and the residue was diluted with ethyl acetate (20 mL) and washed with brine (10 mL*2). The organic layer was dried over anhydrous Na₂SO₄, filtered and concentrated. The residue was purified by prep-TLC to give 14c. MS mass calculated for [M+H]⁺(C₂₅H₂₅Cl₃N₄O₃) requires m/z, 535.1/537.1 LCMS found m/z, 535.0/537.0.

3-(2-chloro-4-(4-((5-cyclopropyl-3-(2,6-dichlorophenyl)isoxazol-4-yl)methoxy)piperidin-1-yl)phenyl)-1,2,4-oxadiazol-5(4H)-one (Compound 14). To a solution of (Z)-2-chloro-4-(4-((5-cyclopropyl-3-(2,6-dichlorophenyl)isoxazol-4-yl)methoxy)piperidin-1-yl)-N′-hydroxybenzimidamide (14c) (150 mg, 279.93 umol) in ethanol (5 mL) was added CH₃ONa (403.28 mg, 2.24 mmol, 30% in MeOH) and diethyl carbonate (1.98 g, 16.80 mmol, 2.03 mL) at 20° C. The mixture was stirred at 80° C. for 12 hours. The reaction mixture was quenched with water (20 mL) and extracted with ethyl acetate (20 mL*2). The combined organic layer was washed with brine (10 mL), dried over anhydrous Na₂SO₄ and filtered and the filtrate was concentrated. The residue was purified by prep-TLC to give Compound 14. MS mass calculated for [M+H]⁺(C₂₆H₂₃Cl₃N₄O₄) requires m/z, 561.1/563.1; LCMS found m/z, 561.1/563.1; ¹H NMR (CHLOROFORM-d, 400 MHz): δ=7.64 (d, J=8.8 Hz, 1H), 7.29-7.35 (m, 2H), 7.20-7.27 (m, 1H), 6.62-6.78 (m, 2H), 4.27 (s, 2H), 3.38-3.47 (m, 1H), 3.19-3.31 (m, 2H), 2.97-3.07 (m, 2H), 2.02-2.12 (m, 1H), 1.63-1.73 (m, 2H), 1.48 (br dd, J=12.6, 3.8 Hz, 2H), 1.17-1.24 (m, 2H), 1.02-1.10 (m, 2H).

### Example 15

### 3-(4-(3-((5-cyclopropyl-3-(2,6-dichlorophenyl)isoxazol-4-yl)methoxy)pyrrolidin-1-yl)phenyl)-1,2,4-oxadiazol-5(4H)-one

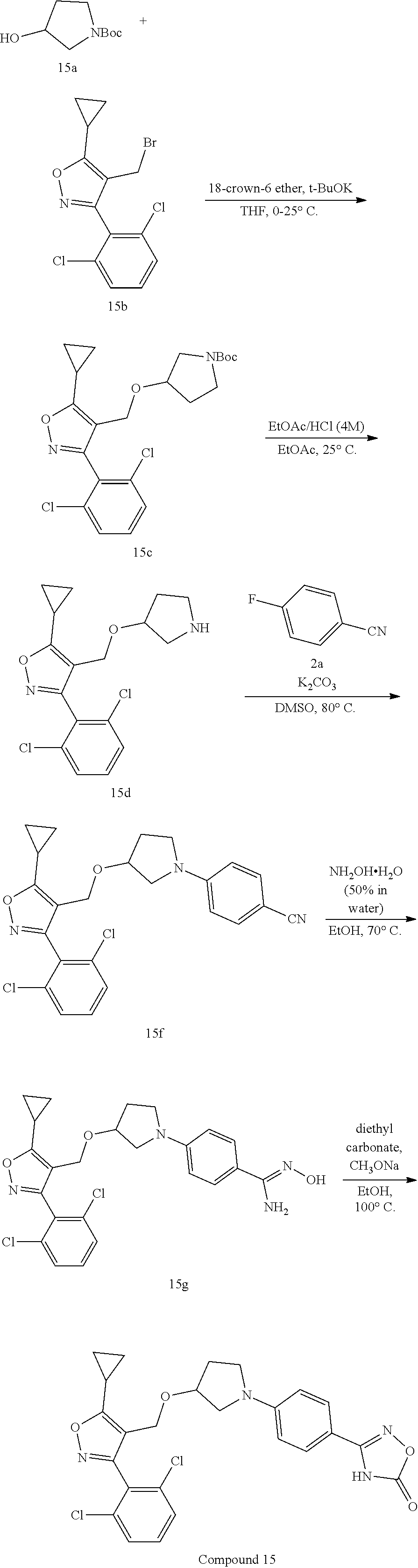

Tert-butyl 3-((5-cyclopropyl-3-(2,6-dichlorophenyl)isoxazol-4-yl)methoxy)pyrrolidine-1-carboxylate (15c). To a solution of tert-butyl 3-hydroxypyrrolidine-1-carboxylate (15a) (50 mg, 267.04 umol) in THF (10 mL) was added 1,4,7,10,13,16-hexaoxacyclooctadecane (105.88 mg, 400.57 umol), t-BuOK (1 M, 400.57 uL) at 0° C. The reaction was degassed and purged with N₂ 3 times and the mixture was stirred at 25° C. for 0.5 hour under N₂ atmosphere. 4-(bromomethyl)-5-cyclopropyl-3-(2,6-dichlorophenyl)-isoxazole (15b) (101.94 mg, 293.75 umol) was added and the mixture was stirred at 25° C. for 15.5 hours. The reaction mixture was concentrated under reduced pressure to remove organic solvent. The residue was diluted with ethyl acetate (5 mL) and water (5 mL), then extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, petroleum ether:ethyl acetate=3:1) to give 15c. MS mass calculated for [M+H]⁺(C₂₂H₂₆Cl₂N₂O₄) requires m/z, 453.1/455.1, LCMS found m/z 453.1/455.1; ¹H NMR (400 MHz, CHLOROFORM-d) δ=7.44-7.39 (m, 2H), 7.38-7.32 (m, 1H), 4.34-4.22 (m, 2H), 3.94 (br s, 1H), 3.41-3.31 (m, 1H), 3.31-3.13 (m, 3H), 2.12 (tt, J=5.1, 8.4 Hz, 1H), 1.77 (br s, 2H), 1.45 (s, 9H), 1.26 (br s, 2H), 1.16-1.10 (m, 2H).

5-cyclopropyl-3-(2,6-dichlorophenyl)-4-((pyrrolidin-3-yloxy)methyl)isoxazole (15d). To a solution of tert-butyl 3-((5-cyclopropyl-3-(2,6-dichlorophenyl)isoxazol-4-yl)methoxy)pyrrolidine-1-carboxylate (15c) (100 mg, 220.58 umol) in ethyl acetate (2 mL) was added HCl/ethyl acetate (2 mL, 4M) at 25° C. and the mixture was stirred at 25° C. for 3 hrs. The reaction mixture was concentrated under reduced pressure to give 15d. ¹H NMR (400 MHz, METHANOL-d4) δ=7.58-7.48 (m, 3H), 4.44-4.32 (m, 2H), 4.18 (br s, 1H), 3.33-3.15 (m, 4H), 2.33-2.26 (m, 1H), 2.03-1.88 (m, 2H), 1.22-1.16 (m, 4H).

4-(3-((5-cyclopropyl-3-(2,6-dichlorophenyl)isoxazol-4-yl)methoxy)pyrrolidin-1-yl)benzonitrile (15f). To a solution of 5-cyclopropyl-3-(2,6-dichlorophenyl)-4-((pyrrolidin-3-yloxy)methyl)isoxazole (15d) (60 mg, 153.96 umol) and 4-fluorobenzonitrile (2a) (186.47 mg, 1.54 mmol) in DMSO (5 mL) was added K₂CO₃ (106.39 mg, 769.82 umol) at 25° C. Then the reaction was degassed and purged with N₂ 3 times and stirred at 80° C. for 16 hours under N₂ atmosphere. The reaction mixture was concentrated under reduced pressure to remove solvent. The residue was diluted with ethyl acetate (5 mL) and water (5 mL), then extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, petroleum ether:ethyl acetate=1:1) to give 15f. MS mass calculated for [M+H]⁺(C₂₄H₂₁Cl₂N₃O₂) requires m/z, 454.1/456.1, LCMS found m/z 454.1/456.1; ¹H NMR (400 MHz, CHLOROFORM-d) δ=7.48-7.42 (m, 2H), 7.31 (dd, J=1.2, 7.8 Hz, 1H), 7.25 (d, J=1.2 Hz, 1H), 7.21-7.16 (m, 1H), 6.40 (d, J=8.8 Hz, 2H), 4.39-4.28 (m, 2H), 4.16-4.12 (m, 1H), 3.37-3.22 (m, 3H), 3.11 (d, J=11.0 Hz, 1H), 2.11 (tt, J=5.1, 8.5 Hz, 1H), 2.07-1.92 (m, 2H), 1.30-1.25 (m, 2H), 1.15-1.09 (m, 2H).

(Z)-4-(3-((5-cyclopropyl-3-(2,6-dichlorophenyl)isoxazol-4-yl)methoxy)pyrrolidin-1-yl)-N′-hydroxybenzimidamide (15g). To a solution of 4-(3-((5-cyclopropyl-3-(2,6-dichlorophenyl)isoxazol-4-yl)methoxy)pyrrolidin-1-yl)-benzonitrile (15f) (60 mg, 132.06 umol) in ethanol (6 mL) was added hydroxylamine (145.40 mg, 2.20 mmol, 0.6 mL, 50% in water) at 25° C. The reaction was degassed and purged with N₂ 3 times, and the mixture was stirred at 70° C. for 16 hours under N₂ atmosphere. The reaction mixture was concentrated under reduced pressure to remove solvent. Water (5 mL) and ethyl acetate (5 mL) were added and the aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, ethyl acetate) to give 15g. MS mass calculated for [M+H]⁺(C₂₄H₂₄Cl₂N₄O₃) requires m/z, 487.1/489.1, LCMS found m/z 487.1/489.0; ¹H NMR (400 MHz, CHLOROFORM-d) δ=7.49 (d, 2H), 7.31 (d, 1H), 7.24 (s, 1H), 7.14-7.21 (m, 1H), 6.42 (d, 2H), 4.81 (br s, 2H), 4.28-4.38 (m, 2H), 4.13 (br d, 1H), 3.34 (dd, 1H), 3.21-3.30 (m, 2H), 3.07 (br d, 1H), 2.08-2.16 (m, 1H), 1.95-2.02 (m, 2H), 1.26-1.29 (m, 2H), 1.12 (br dd, 2H).

3-(4-(3-((5-cyclopropyl-3-(2,6-dichlorophenyl)isoxazol-4-yl)methoxy)pyrrolidin-1-yl)phenyl)-1,2,4-oxadiazol-5(4H)-one (Compound 15). A mixture of (Z)-4-(3-((5-cyclopropyl-3-(2,6-dichlorophenyl)isoxazol-4-yl)methoxy)pyrrolidin-1-yl)-N′-hydroxybenzimidamide (15g) (35 mg, 71.81 umol) in ethanol (3 mL) was added diethyl carbonate (975.00 mg, 8.25 mmol, 1 mL) and CH₃ONa (77.59 mg, 430.88 umol, 30% in MeOH) at 25° C. Then the reaction was degassed and purged with N₂ 3 times and stirred at 100° C. for 24 hours under N₂ atmosphere. The reaction mixture was concentrated under reduced pressure to remove solvent. The residue was diluted with ethyl acetate (5 mL) and water (5 mL), then extracted with ethyl acetate (5 mL*4). The combined organic phase was washed with brine (20 mL*2), dried with anhydrous Na₂SO₄, filtered and concentrated under reduced pressure and purified by prep-HPLC (neutral condition: column: Waters Xbridge BEH C18 100*25 mm*5 um; mobile phase: [water(10 mM NH₄HCO₃)-ACN]; B %: 20%-50%, 10 min) to give Compound 15. MS mass calculated for [M+H]⁺(C₂₅H₂₂Cl₂N₄O₄) requires m/z, 513.1/515.1, LCMS found m/z 513.2/515.2; ¹H NMR (400 MHz, CHLOROFORM-d) δ=7.61 (d, J=8.8 Hz, 2H), 7.32 (dd, J=1.2, 7.9 Hz, 1H), 7.26 (m, 1H), 7.23-7.18 (m, 1H), 6.49 (d, J=8.9 Hz, 2H), 4.34 (q, J=12.0 Hz, 2H), 4.15 (br d, J=2.4 Hz, 1H), 3.40-3.25 (m, 3H), 3.14 (br d, J=10.8 Hz, 1H), 2.12 (tt, J=5.0, 8.4 Hz, 1H), 2.07-1.94 (m, 2H), 1.30-1.24 (m, 2H), 1.16-1.08 (m, 2H).

### Example 16

### 5-(4-(4-((5-cyclopropyl-3-(2,6-dichlorophenyl)isoxazol-4-yl)methoxy)piperidin-1-yl)phenyl)isoxazol-3(2H)-one

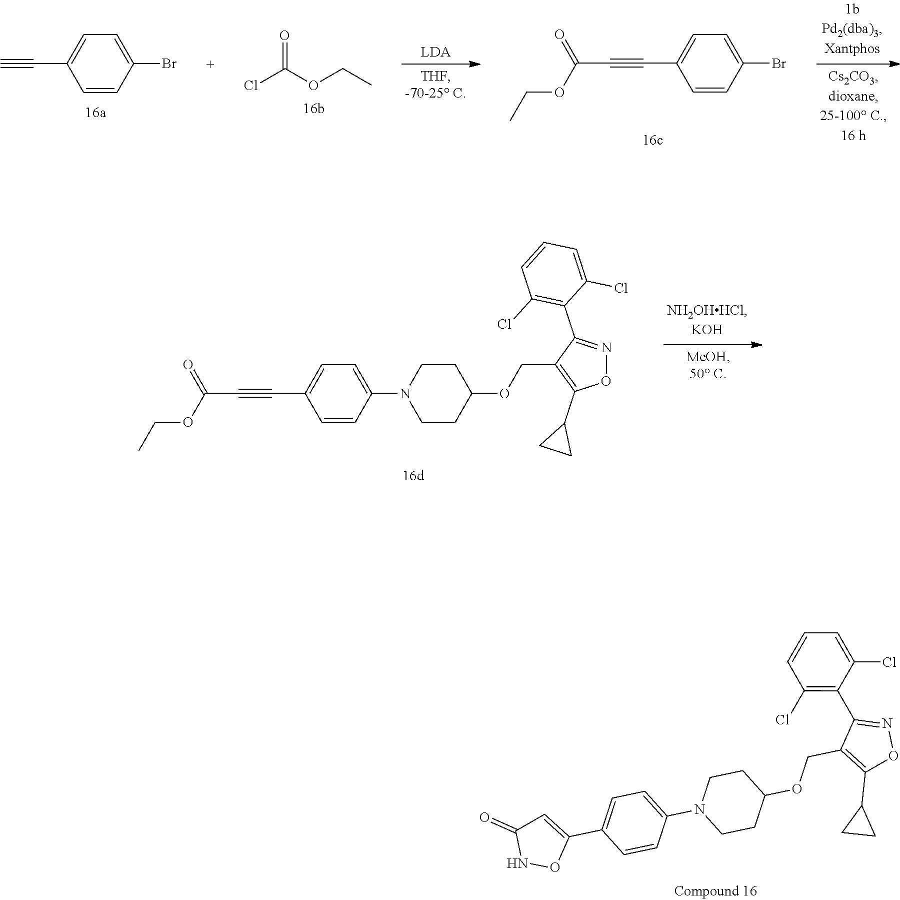

Ethyl 3-(4-bromophenyl)propiolate (16c). To a solution of 1-bromo-4-ethynylbenzene (16a) (1 g, 5.52 mmol) in THE (25 mL) was added LDA (2 M, 6.90 mL) dropwise at −70° C. After addition, the mixture was stirred at this temperature for 0.5 h, and then ethyl carbonochloridate (16b) (2.70 g, 24.86 mmol, 2.37 mL) was added dropwise at −70° C. The resulting mixture was stirred at 25° C. for 5.5 hours. The mixture was quenched with saturated ammonium chloride solution (5 ml). Then water (5 mL) and ethyl acetate (10 mL) were added to the mixture and the phases separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by column chromatography (SiO₂, petroleum ether:ethyl acetate=1:0 to 0:1) to give 16c. ¹H NMR (400 MHz, CHLOROFORM-d) δ=7.56-7.50 (m, 2H), 7.48-7.42 (m, 2H), 4.31 (q, J=7.2 Hz, 2H), 1.36 (t, J=7.2 Hz, 3H).

Ethyl 3-(4-(4-((5-cyclopropyl-3-(2,6-dichlorophenyl)isoxazol-4-yl)methoxy)piperidin-1-yl)phenyl)propiolate (16d). To a solution of 5-cyclopropyl-3-(2,6-dichlorophenyl)-4-((piperidin-4-yloxy)methyl)isoxazole hydrochloride (1b) (100 mg, 272.28 umol) and ethyl 3-(4-bromophenyl)propiolate (16c) (137.82 mg, 544.56 umol) in 1,4-dioxane (5 mL) was added Cs₂CO₃ (266.14 mg, 816.84 umol), Xantphos (31.51 mg, 54.46 umol) and Pd₂(dba)₃ (24.93 mg, 27.23 umol) at 25° C. The reaction was degassed and purged with N₂ 3 times and the mixture was stirred at 100° C. for 16 hours under N₂ atmosphere. The mixture was filtered and the filter cake was washed with dichloromethane (20 mL). The filtrate was concentrated under reduced pressure to remove solvent. The residue was diluted with ethyl acetate (5 mL) and water (5 mL), then extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, petroleum ether:ethyl acetate=3:1) to give 16d. MS mass calculated for [M+H]⁺(C₂₉H₂₈Cl₂N₂O₄) requires m/z, 539.2/541.2, LCMS found m/z 539.2/541.2; ¹H NMR (400 MHz, CHLOROFORM-d) δ=7.46 (d, J=8.8 Hz, 2H), 7.40-7.36 (m, 2H), 7.31-7.27 (m, 1H), 6.77 (d, J=8.8 Hz, 2H), 4.34 (s, 2H), 4.32-4.25 (m, 2H), 3.50-3.43 (m, 1H), 3.38-3.30 (m, 2H), 3.01 (ddd, J=3.5, 8.6, 12.6 Hz, 2H), 2.15 (tt, J=5.0, 8.5 Hz, 1H), 1.81-1.72 (m, 2H), 1.56-1.49 (m, 2H), 1.35 (t, J=7.1 Hz, 3H), 1.29-1.26 (m, 2H), 1.15-1.10 (m, 2H).

5-(4-(4-((5-cyclopropyl-3-(2,6-dichlorophenyl)isoxazol-4-yl)methoxy)piperidin-1-yl)phenyl)isoxazol-3(2H)-one (Compound 16). To a mixture of ethyl 3-(4-(4-((5-cyclopropyl-3-(2,6-dichlorophenyl)isoxazol-4-yl)methoxy)piperidin-1-yl)-phenyl)propiolate (16d) (30 mg, 55.61 umol) in Methanol (5 mL) was added hydroxylamine; hydrochloride (38.65 mg, 556.12 umol) and KOH (56.16 mg, 1.00 mmol) at 25° C. The reaction was stirred at 50° C. for 24 hours. The reaction mixture was concentrated under reduced pressure to remove solvent. The residue was diluted with ethyl acetate (5 mL) and water (5 mL), then extracted with ethyl acetate (5 mL*4). The combined organic phase was washed with brine (10 mL), dried with anhydrous Na₂SO₄, filtered and concentrated under reduced pressure and purified by prep-HPLC (neutral condition: column: Waters Xbridge BEH C18 100*25 mm*5 um; mobile phase: [water(10 mM NH₄HCO₃)-ACN]; B %: 25%-55%, 10 min) to give Compound 16. MS mass calculated for [M+H]⁺(C₂₇H₂₅Cl₂N₃O₄) requires m/z, 526.1/528.1, LCMS found m/z 526.1/528.1; ¹H NMR (400 MHz, CHLOROFORM-d) δ=7.59 (d, J=8.8 Hz, 2H), 7.42-7.37 (m, 2H), 7.32-7.28 (m, 1H), 6.88 (d, J=9.0 Hz, 2H), 6.02 (s, 1H), 4.36 (s, 2H), 3.46 (td, J=3.9, 7.4 Hz, 1H), 3.40-3.32 (m, 2H), 3.00 (ddd, J=3.3, 8.5, 12.4 Hz, 2H), 2.20-2.13 (m, 1H), 1.84-1.76 (m, 2H), 1.57 (dtd, J=3.6, 8.2, 12.4 Hz, 2H), 1.31-1.26 (m, 2H), 1.17-1.11 (m, 2H).

### Example 17

### 2-(4-(4-((5-cyclopropyl-3-(2,6-dichlorophenyl)isoxazol-4-yl)methoxy)piperidin-1-yl)phenyl)-1,2,4-triazine-3, 5(2H,4H)-dione

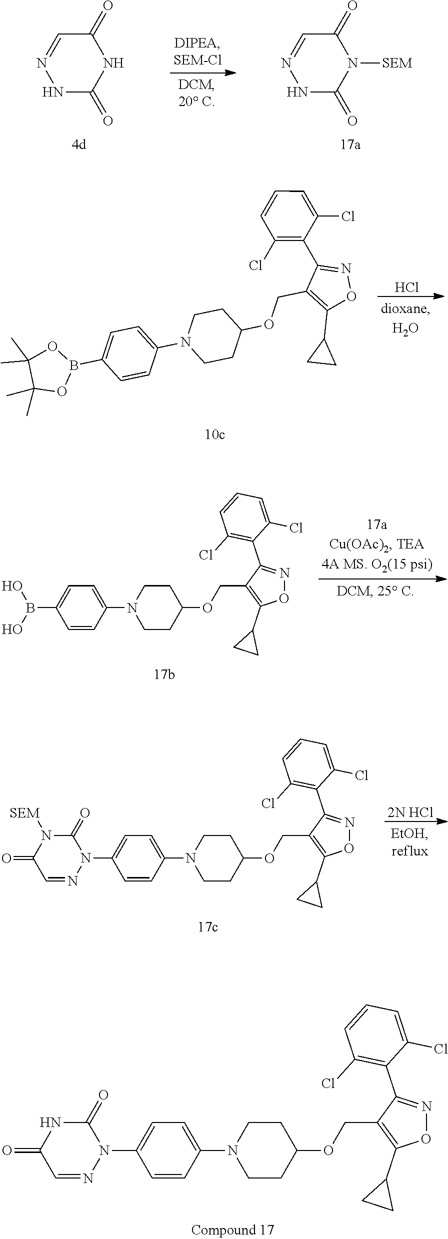

4-((2-(trimethylsilyl)ethoxy)methyl)-1,2,4-triazine-3,5(2H,4H)-dione (17a). To a solution of 4d (1 g, 8.84 mmol) in DCM (10 mL) was added DIPEA (3.43 g, 26.53 mmol, 4.62 mL) and SEM-Cl (1.47 g, 8.84 mmol, 1.57 mL) at 20° C. and the mixture was stirred at 20° C. for 2 hours. The reaction mixture was poured into H₂O (20 mL) and extracted with dichloromethane (20 mL*2). The organic layer was washed with brine (10 mL*2), dried over anhydrous Na₂SO₄, filtered and concentrated under reduced pressure to give a residue. The residue was purified by column chromatography (SiO₂, petroleum ether:ethyl acetate=50:1 to 5:1) and prep-TLC (SiO₂, dichloromethane:Methanol=20:1) to give 17a. MS mass calculated for [M−H]⁻ (C₉H₁₇N₃O₃Si) requires m/z, 242.1, LCMS found m/z, 242.1; ¹H NMR (400 MHz, CHLOROFORM-d) δ=10.11 (br s, 1H), 7.44 (s, 1H), 5.37 (s, 2H), 3.74-3.67 (m, 2H), 1.01-0.95 (m, 2H), 0.04-−0.02 (m, 9H).

(4-(4-((5-cyclopropyl-3-(2,6-dichlorophenyl)isoxazol-4-yl)methoxy)piperidin-1-yl)phenyl)boronic acid (17b). To a solution of 10c (50 mg, 87.82 umol) in 1,4-dioxane (0.5 mL) and H₂O (1.5 mL) was added HCl (6 M in H₂O, 892.86 uL) in one portion at 25° C. under N₂. The mixture was stirred at 25° C. for 12 hours. The residue was purified by prep-HPLC to give 17b. MS mass calculated for [M+H]⁺(C₂₄H₂₅BCl₂N₂O₄) requires m/z, 486.1/489.1, LCMS found m/z, 487.1/489.0.

2-(4-(4-((5-cyclopropyl-3-(2,6-dichlorophenyl)isoxazol-4-yl)methoxy)piperidin-1-yl)phenyl)-4-((2-(trimethylsilyl)ethoxy)methyl)-1,2,4-triazine-3,5(2H,4H)-dione (17c). To a solution of (4-(4-((5-cyclopropyl-3-(2,6-dichlorophenyl)isoxazol-4-yl)methoxy)piperidin-1-yl)phenyl)boronic acid (17b) (20 mg, 41.05 umol) and 4-((2-(trimethylsilyl)ethoxy)methyl)-1,2,4-triazine-3,5(2H,4H)-dione (17a) (19.98 mg, 82.10 umol) in dichloromethane (2 mL) was added Cu(OAc)₂ (8.95 mg, 49.26 umol, TEA (8.31 mg, 82.10 umol, 11.43 uL) and 4 A M.S. (10 mg) in one portion. The resulting mixture was degassed and purged with O₂ 3 times and stirred at 25° C. for 18 hours under O₂ balloon. The reaction mixture was filtered through a Celite pad. The filter cake was rinsed with dichloromethane (10 mL*2). The combined filtrate was washed with water (10 mL) and brine (10 mL), dried over anhydrous Na₂SO₄, filtered and concentrated. The residue was purified by prep-TLC (SiO₂, dichloromethane:Methanol=20:1) to give 17c. MS mass calculated for [M+H]⁺(C₃₃H₃₉Cl₂N₅O₅Si) requires m/z, 684.2/686.2, LCMS found m/z, 684.3/686.3; ¹H NMR (400 MHz, CHLOROFORM-d) δ=7.55 (s, 1H), 7.41-7.37 (m, 2H), 7.36-7.28 (m, 3H), 6.91 (br d, J=8.8 Hz, 2H), 5.44 (s, 2H), 4.35 (s, 2H), 3.78-3.71 (m, 2H), 3.44 (br s, 1H), 3.30 (br s, 2H), 2.93 (br s, 2H), 2.16 (br d, J=5.4 Hz, 1H), 1.80 (br s, 2H), 1.56 (br s, 2H) 1.31-1.27 (m, 2H), 1.16-1.10 (m, 2H), 1.03-0.95 (m, 2H), 0.02 (s, 9H).

2-(4-(4-((5-cyclopropyl-3-(2,6-dichlorophenyl)isoxazol-4-yl)methoxy)piperidin-1-yl)phenyl)-1,2,4-triazine-3,5(2H,4H)-dione (Compound 17). To a solution of 2-(4-(4-((5-cyclopropyl-3-(2,6-dichlorophenyl)isoxazol-4-yl)methoxy)piperidin-1-yl)phenyl)-4-((2-(trimethylsilyl)ethoxy)methyl)-1,2,4-triazine-3,5(2H,4H)-dione (17c) (15 mg, 21.91 umol) in EtOH (0.5 mL) was added aqueous HCl (1.1 mL, 2N), then heated to 50° C. and stirred for 18 hours. The reaction mixture was concentrated under reduced pressure. The residue was purified by prep-HPLC (TFA condition; column: Phenomenex Luna C18 150*30 mm*5 um; mobile phase: [water (0.10% TFA)-ACN]; B %: 40%-70%, 8 min) and lyophilized to give Compound 17. MS mass calculated for [M+H]⁺(C₂₇H₂₅Cl₂N₅O₄) requires m/z, 554.1/556.1, LCMS found m/z, 554.0/556.0; ¹H NMR (400 MHz, CHLOROFORM-d) δ=8.57 (br s, 1H), 7.74-7.55 (m, 3H), 7.46-7.41 (m, 2H), 7.39 (s, 1H), 7.37-7.29 (m, 2H), 4.37 (s, 2H), 3.62 (br s, 1H), 3.38-3.31 (m, 2H), 3.23 (br d, J=12.3 Hz, 2H), 2.24-2.03 (m, 3H), 1.77 (br d, J=10.4 Hz, 2H), 1.34-1.27 (m, 2H), 1.23-1.10 (m, 2H).

### Example 18

### 3-(4-(4-((5-cyclopropyl-3-(2,6-dichlorophenyl)isoxazol-4-yl)methoxy)azepan-1-yl)phenyl)-1,2,4-oxadiazol-5(4H)-one

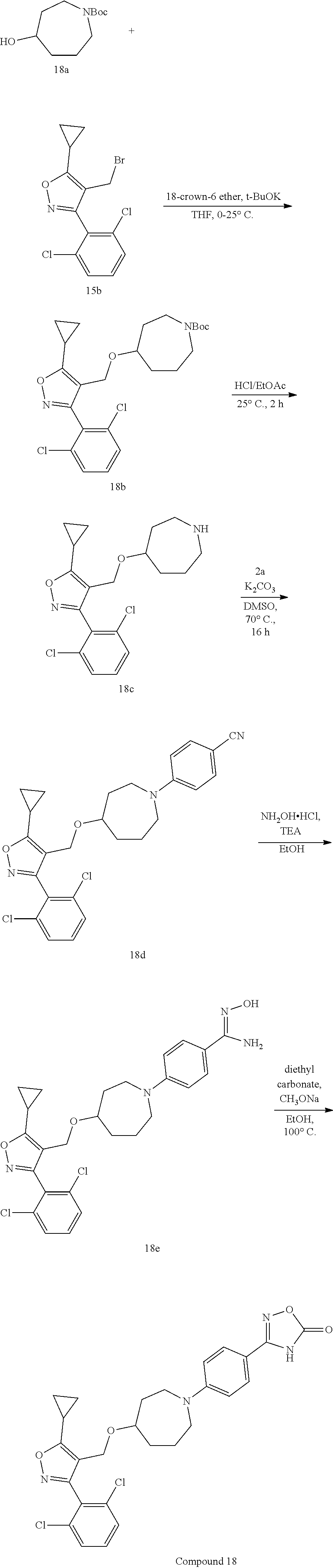

Tert-butyl 4-((5-cyclopropyl-3-(2,6-dichlorophenyl)isoxazol-4-yl)methoxy)azepane-1-carboxylate (18b). To a solution of tert-butyl 4-hydroxyazepane-1-carboxylate (18a) (200 mg, 928.99 umol) in THF (3 mL) was added 18-CROWN-6 (368.32 mg, 1.39 mmol) at 25° C. under N₂ atmosphere. The mixture was degassed and purged with N₂ 3 times, then a solution of t-BuOK (1 M, 1.39 mL) was added dropwise at 0° C. The mixture was stirred at 25° C. for 30 minutes. A solution of 4-(bromomethyl)-5-cyclopropyl-3-(2,6-dichlorophenyl)isoxazole (15b) (322.39 mg, 928.99 umol) in THF (3 mL) was added dropwise at 25° C. The mixture was stirred at 25° C. for 12 hours. The reaction mixture was poured into H₂O (10 mL), the mixture was extracted with ethyl acetate (20 mL*2). The combined organic layer was washed with brine (10 mL), dried over anhydrous Na₂SO₄, filtered and concentrated under reduced pressure to give a residue. The residue was purified by prep-TLC to give 18b. MS mass calculated for [M+H]⁺(C₂₄H₃₀Cl₂N₂O₄) requires m/z, 481.2/483.2, LCMS found m/z, 481.1/483.1; ¹H NMR (400 MHz, CHLOROFORM-d) δ=7.39-7.44 (m, 2H) 7.29-7.38 (m, 1H) 4.21-4.33 (m, 2H) 3.28-3.52 (m, 3H) 3.05-3.25 (m, 2H) 2.09-2.18 (m, 1H) 1.53-1.77 (m, 6H) 1.39-1.49 (m, 9H) 1.23-1.29 (m, 2H) 1.09-1.16 (m, 2H).

4-((azepan-4-yloxy)methyl)-5-cyclopropyl-3-(2,6-dichlorophenyl)isoxazole (18c). A solution of tert-butyl 4-((5-cyclopropyl-3-(2,6-dichlorophenyl)isoxazol-4-yl)methoxy)azepane-1-carboxylate (18b) (270 mg, 560.85 umol) in HCl/ethyl acetate (5 mL) was stirred at 25° C. for 2 hours. The reaction mixture was washed with brine (10 mL), dried over anhydrous Na₂SO₄, filtered and concentrated under reduced pressure to give 18c. MS mass calculated for [M+H]⁺(C₁₉H₂₂Cl₂N₂O₂) requires m/z, 381.1/383.1, LCMS found m/z, 381.1/382.9; ¹H NMR (400 MHz, CHLOROFORM-d) δ=9.40 (br s, 1H) 7.41-7.50 (m, 2H) 7.30-7.41 (m, 1H) 4.19-4.35 (m, 2H) 3.62 (br s, 1H) 2.90-3.26 (m, 4H) 2.03-2.17 (m, 1H) 1.96 (br d, J=19.85 Hz, 2H) 1.78 (br d, J=10.36 Hz, 4H), 1.23-1.34 (m, 2H) 1.14 (br d, J=5.73 Hz, 2H).

4-(4-((5-cyclopropyl-3-(2,6-dichlorophenyl)isoxazol-4-yl)methoxy)azepan-1-yl)benzonitrile (18d). To a mixture of 4-((azepan-4-yloxy)methyl)-5-cyclopropyl-3-(2,6-dichlorophenyl)isoxazole (18c) (180 mg, 472.08 umol) and 4-fluorobenzonitrile (2a) (571.74 mg, 4.72 mmol) in DMSO (3 mL) was added K₂CO₃ (260.97 mg, 1.89 mmol) in one portion at 25° C. under N₂. The mixture was stirred at 70° C. for 12 hours and was poured into ethyl acetate (10 mL). The mixture was washed with water (10 mL*2), brine (10 mL*2), dried with anhydrous Na₂SO₄, filtered and concentrated under reduced pressure. The residue was purified by column chromatography to give 18d. MS mass calculated for [M+H]⁺(C₂₆H₂₅Cl₂N₃O₂) requires m/z, 482.2/483.9, LCMS found m/z, 482.1/484.1; ¹H NMR (400 MHz, CHLOROFORM-d) δ=7.46-7.38 (m, 4H), 7.37-7.31 (m, 1H), 6.59 (d, J=8.9 Hz, 2H), 4.33-4.22 (m, 2H), 3.51-3.44 (m, 1H), 3.43-3.36 (m, 1H), 3.35-3.29 (m, 2H), 3.28-3.20 (m, 1H), 2.16-2.07 (m, 1H), 1.83-1.59 (m, 5H), 1.51-1.42 (m, 1H), 1.31-1.22 (m, 2H), 1.16-1.07 (m, 2H).

(Z)-4-(4-((5-cyclopropyl-3-(2,6-dichlorophenyl)isoxazol-4-yl)methoxy)azepan-1-yl)-N′-hydroxybenzimidamide (18e). To a mixture of 4-(4-((5-cyclopropyl-3-(2,6-dichlorophenyl)isoxazol-4-yl)methoxy)azepan-1-yl)benzonitrile (18d) (100 mg, 207.30 umol) in ethanol (2 mL) was added hydroxylamine (6.85 mg, 207.30 umol, 1 mL) in one portion at 20° C. under N₂. The mixture was stirred at 70° C. for 12 hours. The reaction mixture was concentrated under reduced pressure. The residue was purified by prep-TLC to give 18e. MS mass calculated for [M+H]⁺(C₂₆H₂₈Cl₂N₄O₃) requires m/z, 515.2/517.2, LCMS found m/z, 515.2/517.0.

3-(4-(4-((5-cyclopropyl-3-(2,6-dichlorophenyl)isoxazol-4-yl)methoxy)azepan-1-yl)phenyl)-1,2,4-oxadiazol-5(4H)-one (Compound 18). To a mixture of (Z)-4-(4-((5-cyclopropyl-3-(2,6-dichlorophenyl)isoxazol-4-yl)methoxy)azepan-1-yl)-N′-hydroxybenzimidamide (18e) (65 mg, 126.11 umol) and diethyl carbonate (975.00 mg, 8.25 mmol, 1 mL) in ethanol (1 mL) was added CH₃ONa (136.26 mg, 756.65 umol, 30% in methanol) in one portion at 20° C. under N₂ in a sealed tube. The mixture was stirred at 100° C. for 16 hours and was poured into water (15 mL). The mixture was extracted with ethyl acetate (20 mL). The organic phase was washed with brine (10 mL*2), dried with anhydrous Na₂SO₄, filtered and concentrated under reduced pressure. The residue was purified by prep-HPLC and lyophilized to give Compound 18. MS mass calculated for [M+H]⁺(C₂₇H₂₆Cl₂N₄O₄) requires m/z, 541.1/543.1, LCMS found m/z, 541.2/543.1; ¹H NMR (400 MHz, CHLOROFORM-d) δ=7.58 (d, J=8.9 Hz, 2H), 7.47-7.41 (m, 2H), 7.39-7.33 (m, 1H), 6.70 (d, J=8.9 Hz, 2H), 4.36-4.24 (m, 2H), 3.52-3.41 (m, 2H), 3.41-3.32 (m, 2H), 3.32-3.23 (m, 1H), 2.18-2.09 (m, 1H), 1.86-1.64 (m, 5H), 1.55-1.46 (m, 1H), 1.31-1.25 (m, 2H), 1.17-1.10 (m, 2H).

### Example 19

### 3-(4-(3-((5-cyclopropyl-3-(2,6-dichlorophenyl)isoxazol-4-yl)methoxy)azetidin-1-yl)phenyl)-1,2,4-oxadiazol-5(4H)-one

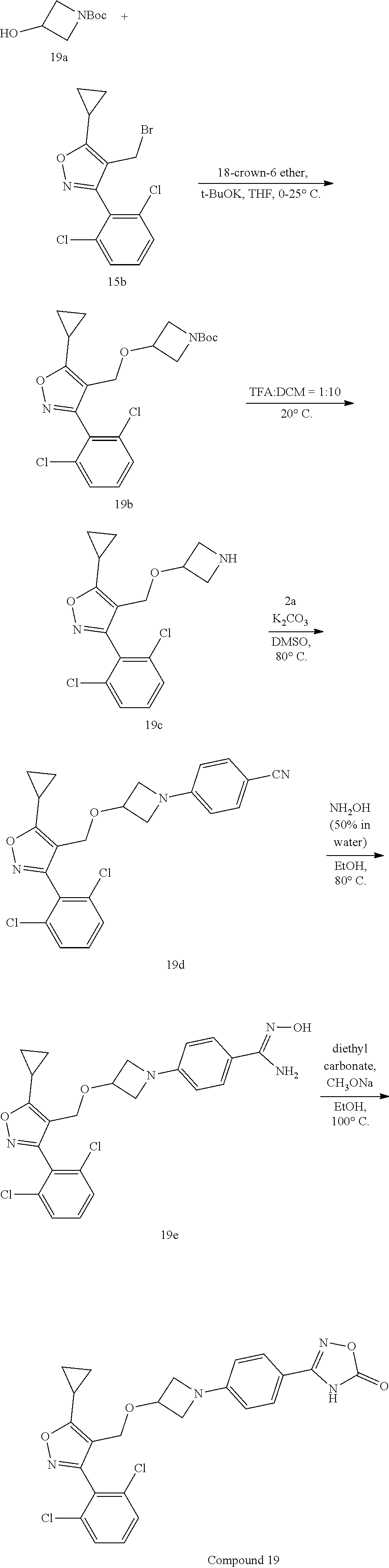

Tert-butyl 3-((5-cyclopropyl-3-(2,6-dichlorophenyl)isoxazol-4-yl)methoxy)azetidine-1-carboxylate (19b). To a solution of tert-butyl 3-hydroxyazetidine-1-carboxylate (19a) (50 mg, 288.67 umol) in THF (10 mL) was added 18-CROWN-6 (114.45 mg, 433.00 umol), t-BuOK (1 M, 433.00 uL) at 0° C. Then the reaction was degassed and purged with N₂ 3 times and stirred at 25° C. for 0.5 hour under N₂ atmosphere. 4-(bromomethyl)-5-cyclopropyl-3-(2,6-dichlorophenyl)isoxazole (15b) (110.20 mg, 317.54 umol) was added. The mixture was stirred at 25° C. for 15.5 hours. The reaction mixture was concentrated under reduced pressure to remove solvent. The residue was diluted with ethyl acetate (5 mL) and water (5 mL), then extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, petroleum ether:ethyl acetate=3:1) to give 19a. ¹H NMR (400 MHz, CHLOROFORM-d) δ=7.46-7.41 (m, 2H), 7.39-7.33 (m, 1H), 4.22 (s, 2H), 4.18-4.11 (m, 1H), 3.93 (dd, J=6.6, 9.2 Hz, 2H), 3.62 (dd, J=4.2, 9.4 Hz, 2H), 2.16-2.07 (m, 1H), 1.43 (s, 9H), 1.31-1.26 (m, 2H), 1.18-1.12 (m, 2H).

4-((azetidin-3-yloxy)methyl)-5-cyclopropyl-3-(2,6-dichlorophenyl)isoxazole (19c). To a solution of tert-butyl 3-((5-cyclopropyl-3-(2,6-dichlorophenyl)isoxazol-4-yl)methoxy)azetidine-1-carboxylate (19b) (60 mg, 136.57 umol) in dichloromethane (6 mL) was added TFA (924.00 mg, 8.10 mmol, 600 uL) at 25° C. and the mixture was stirred at 20° C. for 16 hours. The reaction mixture was concentrated under N₂ to remove dichloromethane. Then saturated sodium bicarbonate solution (5 mL) and dichloromethane (5 mL) were added into the mixture. The mixture was extracted with dichloromethane (5 mL*4). The combined organic phase was washed with brine (20 mL*2), dried with anhydrous Na₂SO₄, filtered and concentrated under reduced pressure to give 19c. MS mass calculated for [M+H]⁺(C₁₆H₁₆Cl₂N₂O₂) requires m/z, 339.1/341.1, LCMS found m/z 339.0/341.1.

4-(3-((5-cyclopropyl-3-(2,6-dichlorophenyl)isoxazol-4-yl)methoxy)azetidin-1-yl)benzonitrile (19d). To a solution of 4-((azetidin-3-yloxy)methyl)-5-cyclopropyl-3-(2,6-dichlorophenyl)isoxazole (19c) (30 mg, 88.44 umol) and 4-fluorobenzonitrile (2a) (107.11 mg, 884.39 umol) in DMSO (2 mL) was added K₂CO₃ (48.89 mg, 353.76 umol) at 25° C. Then the reaction was degassed and purged with N₂ 3 times, and stirred at 80° C. for 16 hours under N₂ atmosphere. The mixture was diluted with ethyl acetate (5 mL) and water (5 mL), then extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, petroleum ether:ethyl acetate=2:1) to give 19d (25 mg, 53.94 umol, 60.99% yield, 95% purity) as colorless oil. MS mass calculated for [M+H]⁺(C₂₃H₁₉Cl₂N₃O₂) requires m/z, 440.1/442.1, LCMS found m/z 440.0/442.0; ¹H NMR (400 MHz, CHLOROFORM-d) δ=7.44 (d, J=8.6 Hz, 2H), 7.41-7.36 (m, 2H), 7.32-7.27 (m, 1H), 6.29 (d, J=8.8 Hz, 2H), 4.41-4.35 (m, 1H), 4.29 (s, 2H), 4.06-4.00 (m, 2H), 3.58 (dd, J=4.3, 8.7 Hz, 2H), 2.13 (tt, J=5.0, 8.4 Hz, 1H), 1.32-1.26 (m, 2H), 1.19-1.13 (m, 2H).

(Z)-4-(3-((5-cyclopropyl-3-(2,6-dichlorophenyl)isoxazol-4-yl)methoxy)azetidin-1-yl)-N′-hydroxybenzimidamide (19e). To a solution of 4-(3-((5-cyclopropyl-3-(2,6-dichlorophenyl)isoxazol-4-yl)methoxy)azetidin-1-yl)-benzonitrile (19d) (25 mg, 56.78 umol) in ethanol (6 mL) was added hydroxylamine (145.40 mg, 2.20 mmol, 0.6 mL, 50% in water) at 25° C. The reaction was degassed and purged with N₂ 3 times and stirred at 80° C. for 16 hours under N₂ atmosphere. The reaction mixture was concentrated under reduced pressure to remove solvent. The residue was diluted with ethyl acetate (5 mL) and water (5 mL), then extracted with ethyl acetate (5 mL*4). The combined organic phase was washed with brine (10 mL*2), dried with anhydrous Na₂SO₄, filtered and concentrated under reduced pressure. The residue was purified by prep-TLC (SiO₂, ethyl acetate) to give 19e. MS mass calculated for [M+H]⁺(C₂₃H₂₂Cl₂N₄O₃) requires m/z, 473.1/475.1, LCMS found m/z 473.0/475.1; ¹H NMR (400 MHz, CHLOROFORM-d) δ=7.47 (d, J=8.6 Hz, 2H), 7.39-7.34 (m, 2H), 7.29 (s, 1H), 7.28-7.27 (m, 1H), 7.25 (s, 1H), 6.35 (d, J=8.6 Hz, 2H), 4.80 (br s, 2H), 4.41-4.33 (m, 1H), 4.28 (s, 2H), 4.03-3.97 (m, 2H), 3.50 (dd, J=4.5, 8.3 Hz, 2H), 2.14 (tt, J=5.1, 8.4 Hz, 1H), 1.29-1.26 (m, 2H), 1.19-1.13 (m, 2H).

3-(4-(3-((5-cyclopropyl-3-(2,6-dichlorophenyl)isoxazol-4-yl)methoxy)azetidin-1-yl)phenyl)-1,2,4-oxadiazol-5(4H)-one (Compound 19). A mixture of (Z)-4-(3-((5-cyclopropyl-3-(2,6-dichlorophenyl)isoxazol-4-yl)methoxy)azetidin-1-yl)-N′-hydroxybenzimidamide (19e) (25 mg, 52.81 umol) in ethanol (3 mL) was added diethyl carbonate (1.95 g, 16.51 mmol, 2 mL) and CH₃ONa (57.06 mg, 316.89 umol, 30% in MeOH) at 25° C. Then the reaction was degassed and purged with N₂ 3 times and the mixture was stirred at 100° C. for 48 hours under N₂ atmosphere. The reaction mixture was concentrated under reduced pressure to remove solvent. The residue was diluted with ethyl acetate (5 mL) and water (5 mL), then extracted with ethyl acetate (5 mL*4). The combined organic layers were washed with brine (20 mL*2), dried with anhydrous Na₂SO₄, filtered and concentrated under reduced pressure and purified by prep-HPLC (neutral condition: column: Waters Xbridge BEH C18 100*25 mm*5 um; mobile phase: [water(10 mM NH₄HCO₃)-ACN]; B %: 20%-50%, 10 min) to give Compound 19. MS mass calculated for [M+H]⁺(C₂₄H₂OCl₂N₄O₄) requires m/z, 499.1/501.1, LCMS found m/z 499.1/501.1; ¹H NMR (400 MHz, CHLOROFORM-d) δ=7.51 (d, J=8.6 Hz, 2H), 7.36-7.32 (m, 2H), 7.29-7.23 (m, 1H), 6.33 (d, J=8.4 Hz, 2H), 4.34 (quin, J=5.2 Hz, 1H), 4.24 (s, 2H), 3.99 (t, J=7.3 Hz, 2H), 3.51 (dd, J=4.2, 8.2 Hz, 2H), 2.15-2.06 (m, 1H), 1.24-1.20 (m, 2H), 1.15-1.09 (m, 2H).

### Example 20

### 3-(4-((2S,4R)-4-((5-cyclopropyl-3-(2,6-dichlorophenyl)isoxazol-4-yl)methoxy)-2-methylpiperidin-1-yl)phenyl)-1,2,4-oxadiazol-5(4H)-one

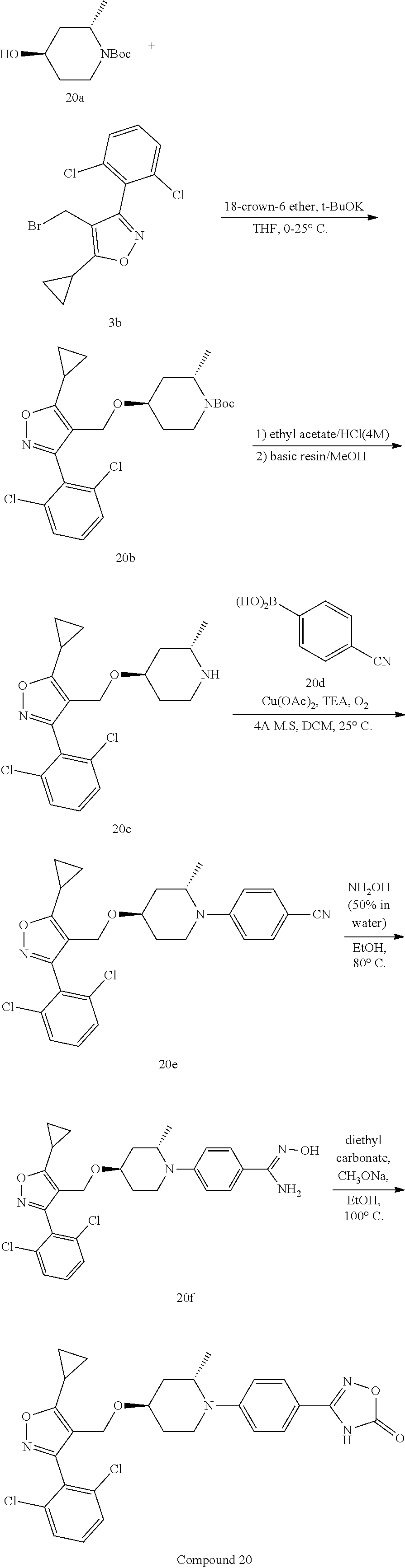

(2S,4R)-tert-butyl 4-((5-cyclopropyl-3-(2,6-dichlorophenyl)isoxazol-4-yl)methoxy)-2-methylpiperidine-1-carboxylate (20b). To a mixture of (2S,4R)-tert-butyl 4-hydroxy-2-methylpiperidine-1-carboxylate (20a) (500 mg, 2.32 mmol) in THF (10 mL) was added 18-crown-6 (920.79 mg, 3.48 mmol) in one portion at 25° C. under N₂, then added t-BuOK (1 M, 3.48 mL) dropwise at 0° C. under N₂. The mixture was stirred at 25° C. for 30 minutes, then 4-(bromomethyl)-5-cyclopropyl-3-(2,6-dichlorophenyl)isoxazole (3b) (805.97 mg, 2.32 mmol) was added. The mixture was stirred at 25° C. for 12 hours. The residue was poured into water (10 mL). The aqueous phase was extracted with ethyl acetate (20 mL). The organic phase was washed with brine (20 mL), dried with anhydrous Na₂SO₄, filtered and concentrated under reduced pressure. The residue was purified by column chromatography (SiO₂, petroleum ether:ethyl acetate=50:1 to 10:1) to give 20b. MS mass calculated for [M+H]⁺(C₂₄H₃₀Cl₂N₂O₄) requires m/z, 481.2/483.2, LCMS found m/z, 481.2/483.0; ¹H NMR (400 MHz, CHLOROFORM-d) δ=7.45-7.39 (m, 2H), 7.37-7.31 (m, 1H), 4.47-4.37 (m, 1H), 4.36-4.26 (m, 2H), 3.95 (br d, J=13.5 Hz, 1H), 3.44 (tt, J=4.2, 11.2 Hz, 1H), 2.73 (dt, J=2.5, 13.5 Hz, 1H), 2.14 (tt, J=5.0, 8.4 Hz, 1H), 1.80-1.71 (m, 1H), 1.65 (td, J=2.1, 12.6 Hz, 1H), 1.51-1.41 (m, 9H), 1.30-1.40 (m, 1H) 1.29-1.23 (m, 2H), 1.15-1.09 (m, 3H), 1.03 (d, J=7.1 Hz, 3H).

5-cyclopropyl-3-(2,6-dichlorophenyl)-4-((((2S,4R)-2-methylpiperidin-4-yl)oxy)methyl)isoxazole (20c). To a solution of (2S,4R)-tert-butyl 4-((5-cyclopropyl-3-(2,6-dichlorophenyl)isoxazol-4-yl)methoxy)-2-methylpiperidine-1-carboxylate (20b) (468 mg, 972.14 umol) in ethyl acetate (5 mL) was added HCl/EtOAc (10 mL, 4M) at 25° C., and the mixture was stirred at 25° C. for 2 hours. The reaction mixture was concentrated under reduced pressure to remove ethyl acetate to give a residue. The residue was diluted with ethyl acetate (5 mL), then added aqueous sodium hydrogen carbonate solution (201.09 mg, 2.39 mmol, 93.10 uL) in one portion at 25° C. under N₂. The mixture was stirred at 25° C. for 10 minutes and was filtered and the filtrate was concentrated under reduced pressure to give 20c. MS mass calculated for [M+H]⁺(C₁₉H₂₂Cl₂N₂O₂) requires m/z, 381.1/383.1 LCMS found m/z, 381.0/382.9;

4-((2S,4R)-4-((5-cyclopropyl-3-(2,6-dichlorophenyl)isoxazol-4-yl)methoxy)-2-methylpiperidin-1-yl)benzonitrile (20e). To a solution of 5-cyclopropyl-3-(2,6-dichlorophenyl)-4-((((2S,4R)-2-methylpiperidin-4-yl)oxy)methyl)-isoxazole (20c) (230 mg, 603.21 umol) and (4-cyanophenyl)boronic acid (20d) (177.27 mg, 1.21 mmol) in dichloromethane (10 mL) was added Cu(OAc)₂ (131.48 mg, 723.85 umol), 4 A M.S. (30 mg), TEA (122.08 mg, 1.21 mmol, 167.92 uL) at 25° C. The suspension was degassed under vacuum and purged with O₂ several times. The mixture was stirred under O₂ ballon at 25° C. for 16 hours. The mixture was filtered and the filter cake was washed by dichloromethane (50 mL). The filtrate was concentrated under reduced pressure to remove solvent. The residue was diluted with ethyl acetate (5 mL) and water (5 mL), then extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, petroleum ether:ethyl acetate=3:1) to give 20e. MS mass calculated for [M+H]⁺(C₂₆H₂₅Cl₂N₃O₂) requires m/z, 482.1/484.1, LCMS found m/z 482.0/484.0; ¹H NMR (400 MHz, CHLOROFORM-d) δ=7.49-7.41 (m, 4H), 7.38-7.33 (m, 1H), 6.78 (d, J=9.2 Hz, 2H), 4.41-4.32 (m, 2H), 4.26 (br t, J=5.2 Hz, 1H), 3.62-3.52 (m, 2H), 2.98-2.87 (m, 1H), 2.21-2.13 (m, 1H), 1.93 (br d, J=12.3 Hz, 1H), 1.82 (td, J=2.1, 12.5 Hz, 1H), 1.39-1.29 (m, 2H), 1.28-1.23 (m, 2H), 1.17-1.11 (m, 2H), 1.04 (d, J=6.8 Hz, 3H).

(Z)-4-((2S,4R)-4-((5-cyclopropyl-3-(2,6-dichlorophenyl)isoxazol-4-yl)methoxy)-2-methylpiperidin-1-yl)-N′-hydroxybenzimidamide (20f). To a solution of 4-((2S,4R)-4-((5-cyclopropyl-3-(2,6-dichlorophenyl)isoxazol-4-yl)methoxy)-2-methylpiperidin-1-yl)benzonitrile (20e) (100 mg, 207.30 umol) in ethanol (6 mL) was added hydroxylamine (3 mL, 50% in water) at 25° C., then the reaction was degassed and purged with N₂ 3 times. The mixture was stirred at 80° C. for 16 hours under N₂ atmosphere. The reaction mixture was concentrated under reduced pressure to remove solvent. The residue was diluted with ethyl acetate (5 mL) and water (5 mL), then extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, dichloromethane:Methanol=10:1) to give 20f. MS mass calculated for [M+H]⁺(C₂₆H₂₈Cl₂N₄O₃) requires m/z, 515.2/517.2, LCMS found m/z 515.1/517.2; ¹H NMR (400 MHz, CHLOROFORM-d) δ=7.49 (d, J=8.8 Hz, 2H), 7.45-7.40 (m, 2H), 7.37-7.31 (m, 1H), 6.84 (d, J=8.8 Hz, 2H), 4.80 (br s, 2H), 4.41-4.31 (m, 2H), 4.15 (br s, 1H), 3.59-3.51 (m, 1H), 3.42 (td, J=4.0, 12.7 Hz, 1H), 2.88 (dt, J=2.8, 12.4 Hz, 1H), 2.21-2.13 (m, 1H), 1.91 (br d, J=11.9 Hz, 1H), 1.79 (br d, J=12.6 Hz, 1H), 1.60 (dt, J=5.1, 11.7 Hz, 1H), 1.44-1.33 (m, 1H), 1.31-1.26 (m, 2H), 1.16-1.10 (m, 2H), 0.96 (d, J=6.8 Hz, 3H).

3-(4-((2S,4R)-4-((5-cyclopropyl-3-(2,6-dichlorophenyl)isoxazol-4-yl)methoxy)-2-methylpiperidin-1-yl)phenyl)-1,2,4-oxadiazol-5(4H)-one (Compound 20). To a mixture of (Z)-4-((2S,4R)-4-((5-cyclopropyl-3-(2,6-dichlorophenyl)isoxazol-4-yl)methoxy)-2-methylpiperidin-1-yl)-N′-hydroxybenzimidamide (20f) (90 mg, 174.61 umol) in ethanol (3 mL) was added diethyl carbonate (1.95 g, 16.51 mmol, 2 mL) and CH₃ONa (188.65 mg, 1.05 mmol, 30% in MeOH) at 25° C. Then the reaction was degassed and purged with N₂ 3 times. The mixture was stirred at 100° C. for 16 hours and was concentrated under reduced pressure to remove solvent. The residue was diluted with ethyl acetate (5 mL) and water (5 mL), then extracted with ethyl acetate (5 mL*4). The combined organic phase was washed with brine (20 mL*2), dried with anhydrous Na₂SO₄, filtered and concentrated under reduced pressure and purified by prep-HPLC (neutral condition: column: Waters Xbridge BEH C18 100*25 mm*5 um; mobile phase: [water(10 mM NH₄HCO₃)-ACN]; B %: 20%-50%, 10 min) to give Compound 20. MS mass calculated for [M+H]⁺(C₂₇H₂₆Cl₂N₄O₄) requires m/z, 541.1/543.1, LCMS found m/z 541.2/543.2; ¹H NMR (400 MHz, CHLOROFORM-d) δ=7.61 (br d, J=8.7 Hz, 2H), 7.47-7.41 (m, 2H), 7.40-7.33 (m, 1H), 6.88 (br d, J=8.8 Hz, 2H), 4.42-4.32 (m, 2H), 4.28 (br s, 1H), 3.63-3.53 (m, 2H), 3.00-2.89 (m, 1H), 2.22-2.13 (m, 1H), 1.94 (br d, J=12.3 Hz, 1H), 1.83 (br d, J=12.6 Hz, 1H), 1.57 (dt, J=5.4, 11.8 Hz, 1H), 1.42-1.33 (m, 1H), 1.32-1.26 (m, 2H), 1.17-1.11 (m, 2H), 1.04 (d, J=6.8 Hz, 3H)

### Example 21

### 3-(4-((2R,4S)-4-((5-cyclopropyl-3-(2,6-dichlorophenyl)isoxazol-4-yl)methoxy)-2-methylpiperidin-1-yl)phenyl)-1,2,4-oxadiazol-5(4H)-one

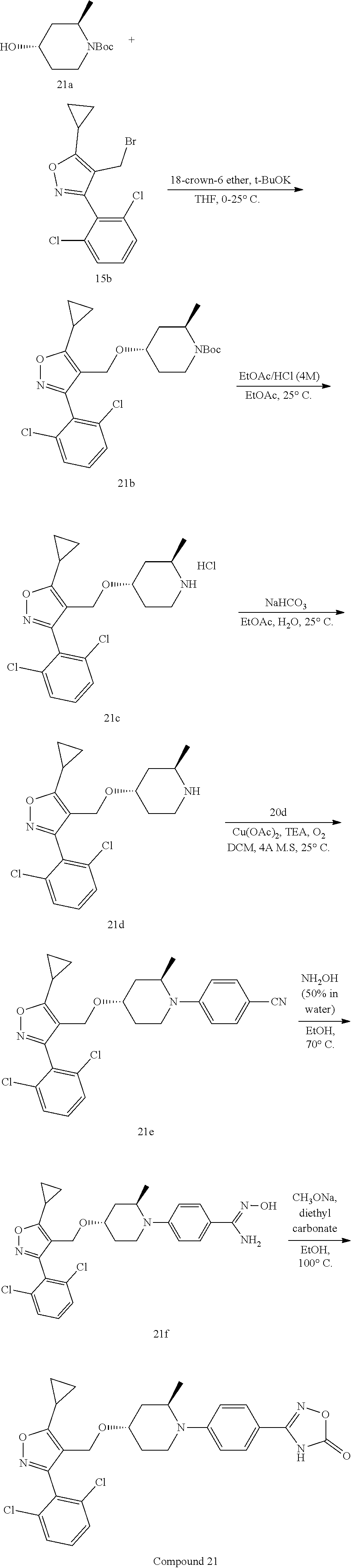

(2R,4S)-tert-butyl 4-((5-cyclopropyl-3-(2,6-dichlorophenyl)isoxazol-4-yl)methoxy)-2-methylpiperidine-1-carboxylate (21b). To a solution of (2R,4S)-tert-butyl 4-hydroxy-2-methylpiperidine-1-carboxylate (21a) (300 mg, 1.39 mmol) in THF (5 mL) was added 18-crown-6 (552.49 mg, 2.09 mmol) and t-BuOK (1 M in THF, 2.09 mL) at 0° C., and the mixture was stirred at 25° C. for 30 minutes, and then 4-(bromomethyl)-5-cyclopropyl-3-(2,6-dichlorophenyl)isoxazole (15b) (483.58 mg, 1.39 mmol) was added to the above solution, the mixture was stirred at 25° C. for 16 hours. The reaction mixture was poured into H₂O (10 mL) and extracted with ethyl acetate (20 mL*2). The organic layer was washed with brine (10 mL), dried over anhydrous Na₂SO₄, filtered and concentrated under reduced pressure to give a residue. The residue was purified by column chromatography (SiO₂, petroleum ether:ethyl acetate=1:0 to 5:1) to give 21b. MS mass calculated for [M+H]⁺(C₂₄H₃₀Cl₂N₂O₄) requires m/z, 481.1/483.2, LCMS found m/z, 481.1/483.2; ¹H NMR (400 MHz, CHLOROFORM-d) δ=7.45-7.40 (m, 2H), 7.38-7.32 (m, 1H), 4.42 (br s, 1H), 4.37-4.27 (m, 2H), 3.96 (br d, J=13.9 Hz, 1H), 3.45 (tt, J=4.4, 11.2 Hz, 1H), 2.74 (dt, J=2.4, 13.6 Hz, 1H), 2.19-2.11 (m, 1H), 1.80-1.72 (m, 1H), 1.66 (td, J=2.0, 12.6 Hz, 1H), 1.44 (s, 9H), 1.36 (dt, J=5.7, 12.0 Hz, 1H), 1.30-1.25 (m, 3H), 1.16-1.09 (m, 2H), 1.04 (d, J=7.1 Hz, 3H).

5-cyclopropyl-3-(2,6-dichlorophenyl)-4-((((2R,4S)-2-methylpiperidin-4-yl)oxy)methyl)isoxazole hydrochloride (21c). To a solution of (2R,4S)-tert-butyl 4-((5-cyclopropyl-3-(2,6-dichlorophenyl)isoxazol-4-yl)methoxy)-2-methylpiperidine-1-carboxylate (21b) (510 mg, 1.06 mmol) in ethyl acetate (5 mL) was added HCl/ethyl acetate (10 mL, 4 M) at 25° C., and the mixture was stirred at 25° C. for 1 hour. The reaction mixture was concentrated under reduced pressure to remove ethyl acetate to give a residue. The residue was triturated with ethyl acetate (5 mL) at 25° C. for 10 minutes, and the mixture was filtered, the filter cake was dried in vacuum to give 21c. MS mass calculated for [M+H]⁺(C₁₉H₂₂Cl₂N₂O₂) requires m/z, 381.1/383.1, LCMS found m/z, 381.1/383.1; ¹H NMR (400 MHz, METHANOL-d4) δ=7.59-7.55 (m, 2H), 7.54-7.49 (m, 1H), 4.44-4.33 (m, 2H), 3.72 (br s, 1H), 3.10 (br dd, J=2.8, 12.7 Hz, 1H), 3.06-2.97 (m, 1H), 2.91 (dt, J=3.1, 13.1 Hz, 1H), 2.31-2.23 (m, 1H), 1.86 (br d, J=14.3 Hz, 2H), 1.76-1.64 (m, 1H), 1.56-1.46 (m, 1H), 1.20 (s, 2H), 1.19-1.15 (m, 5H).

5-cyclopropyl-3-(2,6-dichlorophenyl)-4-((((2R,4S)-2-methylpiperidin-4-yl)oxy)methyl)isoxazole (21d). To a solution of 5-cyclopropyl-3-(2,6-dichlorophenyl)-4-((((2R,4S)-2-methylpiperidin-4-yl)oxy)methyl)-isoxazole (21c) (300 mg, 718.12 umol) in ethyl acetate (10 mL) and H₂O (2 mL) was added sodium bicarbonate (603.27 mg, 7.18 mmol) at 25° C., and the mixture was stirred at 25° C. for 4 hours. The reaction mixture was dried over anhydrous Na₂SO₄, filtered and concentrated under reduced pressure to give 21d.

4-((2R,4S)-4-((5-cyclopropyl-3-(2,6-dichlorophenyl)isoxazol-4-yl)methoxy)-2-methylpiperidin-1-yl)benzonitrile (21e). To a solution of 5-cyclopropyl-3-(2,6-dichlorophenyl)-4-((((2R,4S)-2-methylpiperidin-4-yl)oxy)methyl)-isoxazole (21d) (273 mg, 715.98 umol) and (4-cyanophenyl)boronic acid (20d) (210.41 mg, 1.43 mmol) in dichloromethane (10 mL) were added TEA (144.90 mg, 1.43 mmol), Cu(OAc)₂ (156.06 mg, 859.18 umol) and 4 A M.S. (715.98 umol) at 25° C., and the mixture was stirred at 25° C. for 16 hours under O₂ balloon. The reaction mixture was filtered, the filtrate was poured into H₂O (15 mL) and extracted with dichloromethane (20 mL*3). The organic layers were washed with brine (10 mL), dried over anhydrous Na₂SO₄, filtered and concentrated under reduced pressure to give a residue. The residue was purified by column chromatography (SiO₂, petroleum ether:ethyl acetate=50:1 to 5:1) to give 21e. MS mass calculated for [M+H]⁺(C₂₆H₂₅Cl₂N₃O₂) requires m/z, 482.1/484.1, LCMS found m/z, 482.1/484.1; ¹H NMR (400 MHz, CHLOROFORM-d) δ=7.46 (d, J=9.0 Hz, 2H), 7.44-7.41 (m, 2H), 7.39-7.32 (m, 1H), 6.78 (d, J=9.0 Hz, 2H), 4.41-4.32 (m, 2H), 4.26 (br t, J=5.7 Hz, 1H), 3.62-3.51 (m, 2H), 2.92 (dt, J=3.1, 13.0 Hz, 1H), 2.21-2.12 (m, 1H), 1.97-1.88 (m, 1H), 1.82 (td, J=1.9, 12.7 Hz, 1H), 1.57-1.50 (m, 1H), 1.40-1.31 (m, 1H), 1.30-1.25 (m, 2H), 1.17-1.10 (m, 2H), 1.04 (d, J=6.8 Hz, 3H).

(Z)-4-((2R,4S)-4-((5-cyclopropyl-3-(2,6-dichlorophenyl)isoxazol-4-yl)methoxy)-2-methylpiperidin-1-yl)-N′-hydroxybenzimidamide (21f). To a solution of methyl 4-((2R,4S)-4-((5-cyclopropyl-3-(2,6-dichlorophenyl)isoxazol-4-yl)methoxy)-2-methylpiperidin-1-yl)benzonitrile (21e) (120 mg, 248.76 umol) in ethanol (5 mL) was added hydroxylamine (16.43 mg, 248.76 umol) at 25° C., and the mixture was heated to 70° C. for 16 hours. The reaction mixture was poured into H₂O (10 mL) and extracted with ethyl acetate (15 mL*2). The organic layer was washed with brine (10 mL), dried over Na₂SO₄, filtered and concentrated under reduced pressure to give a residue. The residue was purified by prep-TLC (SiO₂, dichloromethane Methanol=10:1) to give 21f. MS mass calculated for [M+H]⁺(C₂₆H₂₈Cl₂N₄O₃) requires m/z, 515.2/517.2, LCMS found m/z, 515.1/517.2; ¹H NMR (400 MHz, CHLOROFORM-d) δ=7.49 (d, J=8.8 Hz, 2H), 7.45-7.40 (m, 2H), 7.37-7.31 (m, 1H), 6.84 (d, J=8.8 Hz, 2H), 4.81 (br s, 2H), 4.42-4.31 (m, 2H), 4.19-4.14 (m, 1H), 3.60-3.50 (m, 1H), 3.42 (td, J=4.1, 12.7 Hz, 1H), 2.87 (dt, J=2.9, 12.4 Hz, 1H), 2.17 (tt, J=5.1, 8.5 Hz, 1H), 1.95-1.86 (m, 1H), 1.78 (br d, J=11.9 Hz, 1H), 1.65-1.55 (m, 1H), 1.45-1.32 (m, 1H), 1.31-1.27 (m, 2H), 1.17-1.10 (m, 2H), 0.96 (d, J=6.8 Hz, 3H).

3-(4-((2R,4S)-4-((5-cyclopropyl-3-(2,6-dichlorophenyl)isoxazol-4-yl)methoxy)-2-methylpiperidin-1-yl)phenyl)-1,2,4-oxadiazol-5(4H)-one (Compound 21). To a solution of (Z)-4-((2R,4S)-4-((5-cyclopropyl-3-(2,6-dichlorophenyl)isoxazol-4-yl)methoxy)-2-methylpiperidin-1-yl)-N′-hydroxybenzimidamide (21f) (105 mg, 203.71 umol) in ethanol (2 mL) was added CH₃ONa (293.48 mg, 1.63 mmol, 30% in MeOH) and diethyl carbonate (1.95 g, 16.51 mmol, 81.03 eq) at 25° C., the mixture was heated to 100° C. for 16 hours. The reaction mixture was poured into H₂O (10 mL) and extracted with ethyl acetate (15 mL*2). The combined organic layer was washed with brine (10 mL), dried over anhydrous Na₂SO₄, filtered and concentrated under reduced pressure to give a residue. The residue was purified by prep-HPLC (column: Waters Xbridge Prep OBD C18 150*40 mm*10 um; mobile phase: [water (10 mM NH₄HCO₃)-ACN]; B %: 35%-65%, 8 min) to give Compound 21. MS mass calculated for [M+H]⁺(C₂₇H₂₆Cl₂N₄O₄) requires m/z, 541.1/543.1, LCMS found m/z, 541.2/543.2; ¹H NMR (400 MHz, CHLOROFORM-d) δ=7.61 (d, J=8.7 Hz, 2H), 7.48-7.41 (m, 2H), 7.40-7.33 (m, 1H), 6.88 (br d, J=8.8 Hz, 2H), 4.43-4.33 (m, 2H), 4.28 (br s, 1H), 3.64-3.53 (m, 2H), 2.99-2.88 (m, 1H), 2.22-2.13 (m, 1H), 1.94 (br d, J=12.1 Hz, 1H), 1.83 (br d, J=13.0 Hz, 1H), 1.58 (dt, J=5.4, 11.8 Hz, 1H), 1.42-1.33 (m, 1H), 1.32-1.26 (m, 2H), 1.18-1.11 (m, 2H), 1.04 (d, J=6.7 Hz, 3H).

### Example 22

### 3-(4-((2S,4S)-4-((5-cyclopropyl-3-(2,6-dichlorophenyl)isoxazol-4-yl)methoxy)-2-methylpiperidin-1-yl)phenyl)-1,2,4-oxadiazol-5(4H)-one

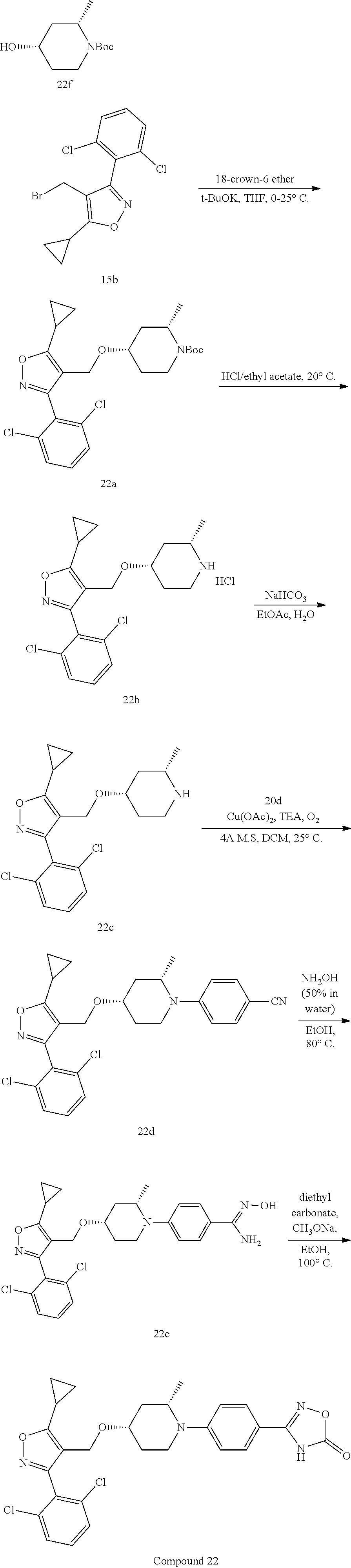

(2S,4S)-tert-butyl 4-((5-cyclopropyl-3-(2,6-dichlorophenyl)isoxazol-4-yl)methoxy)-2-methylpiperidine-1-carboxylate (22a). To a solution of (2S,4S)-tert-butyl 4-hydroxy-2-methylpiperidine-1-carboxylate (22f) (372.22 mg, 1.73 mmol) in THF (10 mL) was added 18-CROWN-6 (685.47 mg, 2.59 mmol) at 20° C., then t-BuOK (1 M in THF, 2.59 mL) was added dropwise at 0° C. The reaction mixture was warmed to 20° C. and stirred for 30 minutes. 4-(bromomethyl)-5-cyclopropyl-3-(2,6-dichlorophenyl)isoxazole (15b) (600 mg, 1.73 mmol) dissolved in THF (5 mL) was added dropwise at this temperature. The resulting mixture was stirred for another 17.5 hours at 20° C. The reaction mixture was quenched by water (10 mL) at 0-10° C., and then extracted with ethyl acetate (30 mL*2). The combined organic phase was washed with brine (10 mL), dried over Na₂SO₄, filtered, and concentrated under reduced pressure. The residue was purified by flash silica gel chromatography to give 22a. MS mass calculated for [M+H]⁺(C₂₄H₃₀Cl₂N₂O₄) requires m/z, 481.2/483.2, LCMS found m/z, 481.1/483.1; ¹H NMR (400 MHz, CHLOROFORM-d) δ=7.41-7.39 (m, 1H), 7.44-7.38 (m, 1H), 7.37-7.30 (m, 1H), 4.33-4.21 (m, 2H), 4.20-4.16 (m, 1H), 3.69 (td, J=2.4, 13.2 Hz, 1H), 3.57 (t, J=3.2 Hz, 1H), 2.96 (dt, J=2.9, 13.2 Hz, 1H), 2.18-2.09 (m, 1H), 1.61 (t, J=3.9 Hz, 2H), 1.52-1.46 (m, 1H), 1.44 (s, 9H), 1.29-1.26 (m, 1H), 1.29-1.24 (m, 2H), 1.15-1.10 (m, 2H), 1.08 (d, J=6.8 Hz, 3H).

5-cyclopropyl-3-(2,6-dichlorophenyl)-4-((((2S,4S)-2-methylpiperidin-4-yl)oxy)methyl)isoxazole (22b). A solution of (2S,4S)-tert-butyl 4-((5-cyclopropyl-3-(2,6-dichlorophenyl)isoxazol-4-yl)methoxy)-2-methylpiperidine-1-carboxylate (22a) (0.83 g, 1.72 mmol) in HCl/EtOAc (4 M, 8.62 mL) was stirred at 20° C. for 2 hours. The reaction mixture was concentrated under reduced pressure. The crude product was triturated with ethyl acetate (10 mL) at 20° C. and stirred for 18 hours, then filtered. The filter cake was dried in vacuum to afford 22b. ¹H NMR (400 MHz, METHANOL-d4) δ=7.63-7.43 (m, 3H), 4.39 (s, 2H), 3.54-3.42 (m, 1H), 3.39-3.32 (m, 1H), 3.15 (ddd, J=2.9, 6.4, 12.3 Hz, 1H), 2.91 (dt, J=2.9, 13.3 Hz, 1H), 2.28 (td, J=6.7, 13.5 Hz, 1H), 2.11-1.98 (m, 2H), 1.43-1.31 (m, 1H), 1.28 (d, J=6.6 Hz, 3H), 1.23-1.10 (m, 5H).

5-cyclopropyl-3-(2,6-dichlorophenyl)-4-((((2S,4S)-2-methylpiperidin-4-yl)oxy)methyl)isoxazole (22c). To a suspension of 5-cyclopropyl-3-(2,6-dichlorophenyl)-4-((((2S,4S)-2-methylpiperidin-4-yl)oxy)methyl)isoxazole hydrochloride (22b) (590 mg, 1.41 mmol) in ethyl acetate (5 mL) was added NaHCO₃(1.19 g, 14.12 mmol, 549.27 uL) in H₂O (2 mL) at 20° C. and the mixture was stirred for 4 hours. The reaction mixture was dried over Na₂SO₄ and filtered. The filter cake was rinsed with ethyl acetate (20 mL*2) and the combined filtrate was concentrated under reduced pressure to give 22c. ¹H NMR (400 MHz, METHANOL-d4) δ=7.60-7.43 (m, 3H), 4.35 (s, 2H), 3.28-3.16 (m, 1H), 3.02-2.89 (m, 1H), 2.55-2.38 (m, 2H), 2.27 (quin, J=6.7 Hz, 1H), 1.83-1.68 (m, 2H), 1.21-1.14 (m, 4H), 1.12-1.06 (m, 1H), 1.03 (d, J=6.4 Hz, 3H), 0.86-0.72 (m, 1H).

4-((2S,4S)-4-((5-cyclopropyl-3-(2,6-dichlorophenyl)isoxazol-4-yl)methoxy)-2-methylpiperidin-1-yl)benzonitrile (22d). To a solution of 5-cyclopropyl-3-(2,6-dichlorophenyl)-4-((((2S,4S)-2-methylpiperidin-4-yl)oxy)methyl)-isoxazole (22c) (210 mg, 550.75 umol) and (4-cyanophenyl)boronic acid (20d) (242.78 mg, 1.65 mmol) in dichloromethane (10 mL) was added Cu(OAc)₂ (120.04 mg, 660.91 umol), 4 A M.S. (50 mg), TEA (111.46 mg, 1.10 mmol, 153.32 uL) at 25° C. The suspension was degassed and purged with O₂ for several times. The mixture was stirred under O₂ balloon at 25° C. for 16 hours. The mixture was filtered and the filter cake was washed by dichloromethane (50 mL). The filtrate was concentrated under reduced pressure to remove solvent. The residue was diluted by ethyl acetate (5 mL) and water (5 mL), extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, petroleum ether:ethyl acetate=3:1) to give 22d. MS mass calculated for [M+H]⁺(C₂₆H₂₅Cl₂N₃O₂) requires m/z, 482.1/484.1, LCMS found m/z 482.1/484.1; ¹H NMR (400 MHz, CHLOROFORM-d) δ=7.48-7.39 (m, 4H), 7.37-7.31 (m, 1H), 6.77 (d, J=8.9 Hz, 2H), 4.37-4.27 (m, 2H), 3.94 (dt, J=3.1, 6.4 Hz, 1H), 3.61 (quin, J=3.7 Hz, 1H), 3.28 (td, J=4.1, 12.8 Hz, 1H), 3.18-3.10 (m, 1H), 3.18-3.10 (m, 1H), 2.15 (tt, J=5.1, 8.5 Hz, 1H), 1.83-1.76 (m, 1H), 1.76-1.64 (m, 3H), 1.28 (dd, J=2.4, 5.0 Hz, 2H), 1.16-1.11 (m, 2H), 1.09 (d, J=6.8 Hz, 3H).

(E)-4-((2S,4S)-4-((5-cyclopropyl-3-(2,6-dichlorophenyl)isoxazol-4-yl)methoxy)-2-methylpiperidin-1-yl)-N′-hydroxybenzimidamide (22e). To a solution of 4-((2S,4S)-4-((5-cyclopropyl-3-(2,6-dichlorophenyl)isoxazol-4-yl)methoxy)-2-methylpiperidin-1-yl)benzonitrile (22d) (60 mg, 124.38 umol) in ethanol (10 mL) was added hydroxylamine (3 mL, 50% in water) at 25° C. The reaction mixture was degassed and purged with N₂ 3 times, and then heated to 80° C. for 16 hours under N₂ atmosphere. The reaction mixture was concentrated under reduced pressure to remove solvent. The residue was diluted by ethyl acetate (5 mL) and water (5 mL), extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, dichloromethane:methanol=10:1) to give 22e. MS mass calculated for [M+H]⁺(C₂₆H₂₈Cl₂N₄O₃) requires m/z, 515.2/517.2, LCMS found m/z 515.1/517.1; ¹H NMR (400 MHz, CHLOROFORM-d) δ=7.48-7.39 (m, 4H), 7.37-7.31 (m, 1H), 6.77 (d, J=8.9 Hz, 2H), 4.37-4.27 (m, 2H), 3.94 (dt, J=3.1, 6.4 Hz, 1H), 3.61 (quin, J=3.7 Hz, 1H), 3.28 (td, J=4.1, 12.8 Hz, 1H), 3.18-3.10 (m, 1H), 3.18-3.10 (m, 1H), 2.15 (tt, J=5.1, 8.5 Hz, 1H), 1.83-1.76 (m, 1H), 1.76-1.64 (m, 3H), 1.28 (dd, J=2.4, 5.0 Hz, 2H), 1.16-1.11 (m, 2H), 1.09 (d, J=6.8 Hz, 3H).

3-(4-((2S,4S)-4-((5-cyclopropyl-3-(2,6-dichlorophenyl)isoxazol-4-yl)methoxy)-2-methylpiperidin-1-yl)phenyl)-1,2,4-oxadiazol-5(4H)-one (Compound 22). To a solution of (E)-4-((2S,4S)-4-((5-cyclopropyl-3-(2,6-dichlorophenyl)isoxazol-4-yl)methoxy)-2-methylpiperidin-1-yl)-N′-hydroxybenzimidamide (22e) (50 mg, 97.01 umol) in ethanol (5 mL) was added diethyl carbonate (3.90 g, 33.01 mmol, 4 mL) and CH₃ONa (174.69 mg, 970.06 umol, 30% in MeOH) at 25° C. in a sealed tube. Then the reaction mixture was stirred at 100° C. for 16 hours and was concentrated under reduced pressure to remove solvent. The residue was diluted by ethyl acetate (5 mL) and water (5 mL), extracted with ethyl acetate (5 mL*4). The combined organic phase was washed with brine (20 mL*2), dried with anhydrous Na₂SO₄, filtered and concentrated under reduced pressure, the residue was purified by prep-HPLC (neutral condition: column: Waters Xbridge BEH C18 100*25 mm*5 um; mobile phase: [water(10 mM NH₄HCO₃)-ACN]; B %: 20%-50%, 10 min) to give Compound 22. MS mass calculated for [M+H]⁺(C₂₇H₂₆Cl₂N₄O₄) requires m/z, 541.1/543.1, LCMS found m/z 541.2/543.2; ¹H NMR (400 MHz, CHLOROFORM-d) δ=7.60 (d, J=8.9 Hz, 2H), 7.45-7.40 (m, 2H), 7.37-7.32 (m, 1H), 6.87 (d, J=9.1 Hz, 2H), 4.38-4.29 (m, 2H), 3.98-3.90 (m, 1H), 3.62 (br t, J=3.8 Hz, 1H), 3.27 (td, J=4.2, 12.7 Hz, 1H), 3.18-3.09 (m, 1H), 2.16 (tt, J=5.1, 8.5 Hz, 1H), 1.85-1.78 (m, 1H), 1.78-1.68 (m, 3H), 1.31-1.25 (m, 2H), 1.17-1.11 (m, 2H), 1.08 (d, J=6.8 Hz, 3H).

### Example 23

### 3-(4-((2R,4R)-4-((5-cyclopropyl-3-(2,6-dichlorophenyl)isoxazol-4-yl)methoxy)-2-methylpiperidin-1-yl)phenyl)-1,2,4-oxadiazol-5(4H)-one

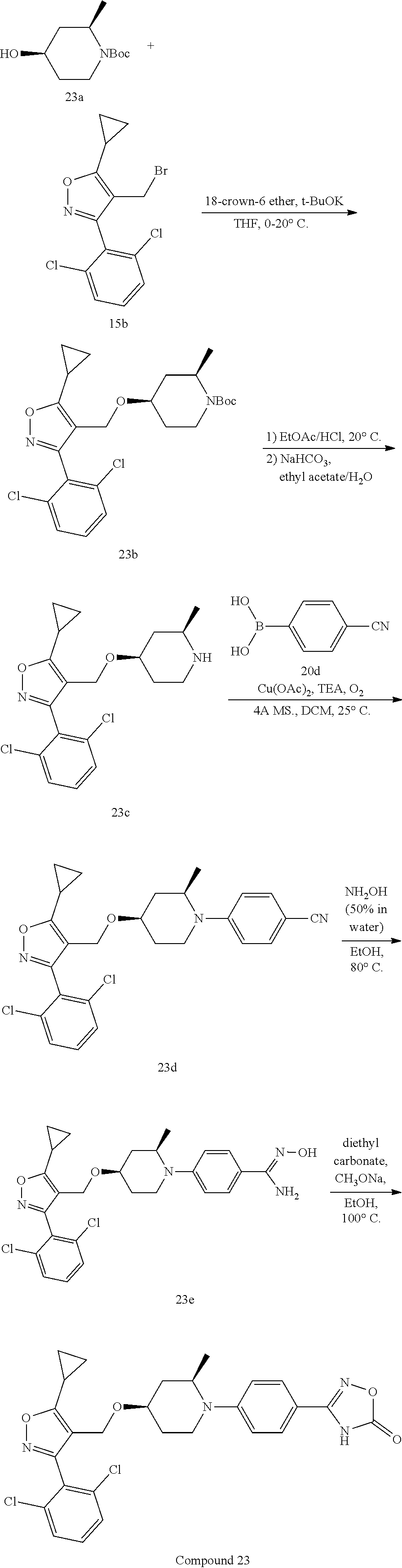

(2R,4R)-tert-butyl 4-((5-cyclopropyl-3-(2,6-dichlorophenyl)isoxazol-4-yl)methoxy)-2-methylpiperidine-1-carboxylate (23b). To a solution of (2R,4R)-tert-butyl 4-hydroxy-2-methylpiperidine-1-carboxylate (23a) (0.2 g, 928.99 umol) in THF (3 mL) was added 18-CROWN-6 (368.32 mg, 1.39 mmol) and t-BuOK (1 M in THF, 1.39 mL) at 25° C. The mixture was stirred for 1 hour. Then 4-(bromomethyl)-5-cyclopropyl-3-(2,6-dichlorophenyl)isoxazole (15b) (354.63 mg, 1.02 mmol) was added to the mixture at 25° C. The mixture was stirred at 25° C. for 12 hours then poured into water (5 mL). The mixture was extracted with ethyl acetate (5 mL*2). The combined organic phase was washed with brine (5 mL*2), dried with anhydrous Na₂SO₄, filtered, and concentrated under reduced pressure. The residue was purified by prep-TLC (petroleum ether:ethyl acetate=3:1) to give 23b. MS mass calculated for [M+H]⁺(C₂₄H₃₀Cl₂N₂O₄) requires m/z, 481.2/483.2, LCMS found m/z, 481.0/483.0; ¹H NMR (400 MHz, CHLOROFORM-d) δ=7.38-7.43 (m, 2H) 7.30-7.36 (m, 1H) 4.21-4.33 (m, 2H) 4.12-4.20 (m, 1H) 3.69 (dt, J=13.3, 2.3 Hz, 1H) 3.54-3.59 (m, 1H) 2.95 (td, J=13.2, 2.9 Hz, 1H) 2.04-2.10 (m, 1H) 1.55-1.64 (m, 4H) 1.44 (s, 9H) 1.23-1.29 (m, 2H) 1.10-1.15 (m, 2H) 1.08 (d, J=7.0 Hz, 3H).

5-cyclopropyl-3-(2,6-dichlorophenyl)-4-((((2R,4R)-2-methylpiperidin-4-yl)oxy)methyl)isoxazole (23c). The solution of (2R,4R)-tert-butyl 4-((5-cyclopropyl-3-(2,6-dichlorophenyl)isoxazol-4-yl)methoxy)-2-methylpiperidine-1-carboxylate (23b) (0.4 g, 830.89 umol) in HCl/EtOAc (5 mL, 4 M) was stirred at 25° C. for 2 hours. The mixture was concentrated under reduced pressure and the resulting residue was diluted with ethyl acetate (10 mL) and sodium bicarbonate solution (5 mL) and stirred for 30 min. The mixture was separated, the aqueous was extracted with ethyl acetate (5 mL*2), the combined organic layer was washed with brine, dried over sodium sulfate and filtered, the filtrate was concentrated to give 23c; MS mass calculated for [M+H]⁺(C₁₉H₂₂Cl₂N₂O₂) requires m/z, 381.1/383.1, LCMS found m/z, 381.0/383.0.

4-((2R,4R)-4-((5-cyclopropyl-3-(2,6-dichlorophenyl)isoxazol-4-yl)methoxy)-2-methylpiperidin-1-yl)benzonitrile (23d). To a mixture of 5-cyclopropyl-3-(2,6-dichlorophenyl)-4-((((2R,4R)-2-methylpiperidin-4-yl)oxy)methyl)-isoxazole (23c) (130 mg, 340.94 umol) and (4-cyanophenyl)boronic acid (20d) (150.29 mg, 1.02 mmol) in dichloromethane (10 mL) was added Cu(OAc)₂ (74.31 mg, 409.13 umol) and TEA (69.00 mg, 681.89 umol, 94.91 uL) in one portion at 20° C. under N₂. The mixture was stirred at 20° C. for 12 hours. The reaction mixture was filtered and the filtrate was washed with water (10 mL). The organic phase was washed with brine (10 mL*2), dried over anhydrous Na₂SO₄, filtered and concentrated under reduced pressure. The residue was purified by prep-TLC to give 23d. MS mass calculated for [M+H]⁺(C₂₆H₂₅Cl₂N₃O₂) requires m/z, 482.1/484.1, LCMS found m/z, 482.2/484.2.

(Z)-4-((2R,4R)-4-((5-cyclopropyl-3-(2,6-dichlorophenyl)isoxazol-4-yl)methoxy)-2-methylpiperidin-1-yl)-N′-hydroxybenzimidamide (23e). To a mixture of 4-((2R,4R)-4-((5-cyclopropyl-3-(2,6-dichlorophenyl)isoxazol-4-yl)methoxy)-2-methylpiperidin-1-yl)benzonitrile (23d) (45 mg, 93.28 umol) in ethanol (3 mL) was added hydroxylamine (3.08 mg, 93.28 umol, 1 mL, 50% in water) in one portion at 25° C. under N₂. The mixture was stirred at 80° C. for 12 hours. The reaction mixture was concentrated under reduced pressure. The residue was purified by prep-TLC to give 23e. MS mass calculated for [M+H]⁺(C₂₆H₂₈Cl₂N₄O₃) requires m/z, 515.2/517.2, LCMS found m/z, 515.0/517.0; ¹H NMR (400 MHz, CHLOROFORM-d) δ=7.50 (d, J=8.7 Hz, 2H), 7.45-7.39 (m, 2H), 7.37-7.31 (m, 1H), 6.91 (d, J=8.7 Hz, 2H), 4.80 (br s, 2H), 4.39-4.26 (m, 2H), 3.58-3.46 (m, 2H), 3.13-3.05 (m, 1H), 3.01-2.92 (m, 1H), 2.21-2.12 (m, 1H), 1.87-1.80 (m, 1H), 1.79-1.61 (m, 1H), 1.60-1.51 (m, 2H), 1.28 (dd, J=2.3, 5.0 Hz, 2H), 1.16-1.10 (m, 2H), 0.98 (d, J=6.5 Hz, 3H).

3-(4-((2R,4R)-4-((5-cyclopropyl-3-(2,6-dichlorophenyl)isoxazol-4-yl)methoxy)-2-methylpiperidin-1-yl)phenyl)-1,2,4-oxadiazol-5(4H)-one (Compound 23). To a mixture of (Z)-4-((2R,4R)-4-((5-cyclopropyl-3-(2,6-dichlorophenyl)isoxazol-4-yl)methoxy)-2-methylpiperidin-1-yl)-N′-hydroxybenzimidamide (23e) (30 mg, 58.20 umol) and diethyl carbonate (975.00 mg, 8.25 mmol, 1 mL) in ethanol (2 mL) was added CH₃ONa (62.89 mg, 349.22 umol, 30% in MeOH) in one portion at 20° C. under N₂. The mixture was stirred at 100° C. for 16 hours. The residue was poured into water (10 mL). The aqueous phase was extracted with ethyl acetate (20 mL). The organic phase was washed with brine (10 mL), dried with anhydrous Na₂SO₄, filtered and concentrated under reduced pressure. The residue was purified by prep-TLC to give Compound 23. MS mass calculated for [M+H]⁺(C₂₇H₂₈Cl₂N₄O₄) requires m/z, 541.1/543.1, LCMS found m/z, 541.2/543.2; ¹H NMR (400 MHz, CHLOROFORM-d) δ=7.53 (br d, J=8.3 Hz, 2H), 7.43-7.36 (m, 2H), 7.35-7.29 (m, 1H), 6.75 (br d, J=7.7 Hz, 2H), 4.37-4.25 (m, 2H), 3.85-3.73 (m, 1H), 3.56 (br s, 1H), 3.19-2.98 (m, 2H), 2.18-2.10 (m, 1H), 1.77-1.73 (m, 1H) 1.72-1.61 (m, 3H), 1.31-1.23 (m, 2H), 1.15-1.08 (m, 2H), 0.99 (br d, J=6.2 Hz, 3H).

### Example 24. 2-(4-(4-((5-cyclopropyl-3-(2,6-dichlorophenyl)isoxazol-4-yl)methoxy)piperidin-1-yl)phenyl)-3,5-dioxo-2,3,4,5-tetrahydro-1,2,4-triazine-6-carbonitrile

### Example 25. 4-(4-(4-((5-cyclopropyl-3-(2,6-dichlorophenyl)isoxazol-4-yl)methoxy)piperidin-1-yl)phenyl)-3,5-dioxo-2,3,4,5-tetrahydro-1,2,4-triazine-6-carbonitrile

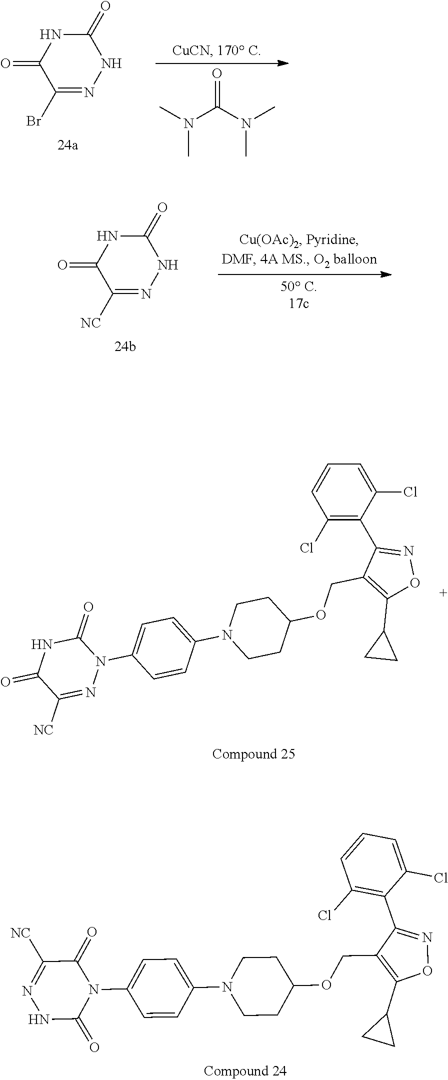

3,5-dioxo-2,3,4,5-tetrahydro-1,2,4-triazine-6-carbonitrile (24b). To a solution of 6-bromo-1,2,4-triazine-3,5(2H,4H)-dione (24a) (1 g, 5.21 mmol) in 1,1,3,3-tetramethylurea (6 mL) was added CuCN (933.09 mg, 10.42 mmol, 2.28 mL) at 25° C. The mixture was stirred at 25° C. for 12 hours and was poured into water (20 mL). Ethyl acetate (20 mL) was added, the slurry was filtered and the filtrate was separated. The aqueous phase was extracted with ethyl acetate (20 mL*2). The combined organic phase was washed with brine (10 mL), dried over anhydrous Na₂SO₄ and concentrated. The residue was purified by silica gel column chromatography to give 24b. MS mass calculated for [M−H]⁻ (C₄H₂N₄O₂) requires m/z, 137.0, LCMS found m/z, 137.0.

2-(4-(4-((5-cyclopropyl-3-(2,6-dichlorophenyl)isoxazol-4-yl)methoxy)piperidin-1-yl)phenyl)-3,5-dioxo-2,3,4,5-tetrahydro-1,2,4-triazine-6-carbonitrile (Compound 24) & 4-(4-(4-((5-cyclopropyl-3-(2,6-dichlorophenyl)isoxazol-4-yl)methoxy)piperidin-1-yl)phenyl)-3,5-dioxo-2,3,4,5-tetrahydro-1,2,4-triazine-6-carbonitrile (Compound 25). To a mixture of 3,5-dioxo-2,3,4,5-tetrahydro-1,2,4-triazine-6-carbonitrile (24b) (11.34 mg, 82.10 umol) and (4-(4-((5-cyclopropyl-3-(2,6-dichlorophenyl)isoxazol-4-yl)methoxy)piperidin-1-yl)phenyl)boronic acid (17c) (20 mg, 41.05 umol) in DMF (1 mL) was added Cu(OAc)₂ (7.46 mg, 41.05 umol), pyridine (6.49 mg, 82.10 umol, 6.63 uL) and 4 A M.S. (20 mg) at 20° C. The mixture was stirred at 50° C. for 12 hours under O₂ balloon. The reaction mixture was poured into water (10 mL) and was extracted with ethyl acetate (10 mL). The organic phase was washed with brine (10 mL*2), dried with anhydrous Na₂SO₄, filtered and concentrated under reduced pressure. The residue was purified by prep-TLC and prep-HPLC (TFA condition) to give Compound 25: MS mass calculated for [M+H]⁺(C₂₈H₂₄Cl₂N₆O₄) requires m/z, 579.1/581.1, LCMS found m/z, 579.2/581.2; ¹H NMR (400 MHz, CHLOROFORM-d) δ=7.41-7.36 (m, 2H), 7.32-7.28 (m, 1H), 7.07 (d, J=9.0 Hz, 2H), 6.93 (d, J=9.0 Hz, 2H), 4.35 (s, 2H), 3.45 (tt, J=3.6, 7.5 Hz, 1H), 3.35-3.28 (m, 2H), 2.97 (ddd, J=3.4, 8.7, 12.4 Hz, 2H), 2.16 (tt, J=5.1, 8.4 Hz, 1H), 1.84-1.75 (m, 2H), 1.57 (ddd, J=3.7, 8.3, 12.4 Hz, 2H), 1.31-1.26 (m, 2H), 1.17-1.11 (m, 2H) and Compound 24: MS mass calculated for [M+H]⁺(C₂₈H₂₄Cl₂N₆O₄) requires m/z, 579.1/581.1, LCMS found m/z, 579.2/581.2; ¹H NMR (400 MHz, CHLOROFORM-d) δ=7.41-7.37 (m, 2H), 7.32-7.28 (m, 3H), 6.88 (br d, J=8.9 Hz, 2H), 4.35 (s, 2H), 3.45 (td, J=3.8, 7.5 Hz, 1H), 3.31 (br d, J=5.3 Hz, 2H), 2.96 (br t, J=9.0 Hz, 2H), 2.21-2.10 (m, 1H), 1.84-1.74 (m, 2H), 1.61-1.50 (m, 2H), 1.31-1.24 (m, 2H), 1.16-1.10 (m, 2H).

### Example 26. 3-(4-(4-((5-cyclopropyl-3-(2,6-difluorophenyl)isoxazol-4-yl)methoxy)piperidin-1-yl)phenyl)-1,2,4-oxadiazol-5(4H)-one

### Example 27. 3-(4-(4-((5-cyclopropyl-3-(2-ethoxy-6-fluorophenyl)isoxazol-4-yl)methoxy)piperidin-1-yl)phenyl)-1,2,4-oxadiazol-5(4H)-one

### Example 28. 3-(4-(4-((5-cyclopropyl-3-(2-fluoro-6-methoxyphenyl)isoxazol-4-yl)methoxy)piperidin-1-yl)phenyl)-1,2,4-oxadiazol-5(4H)-one

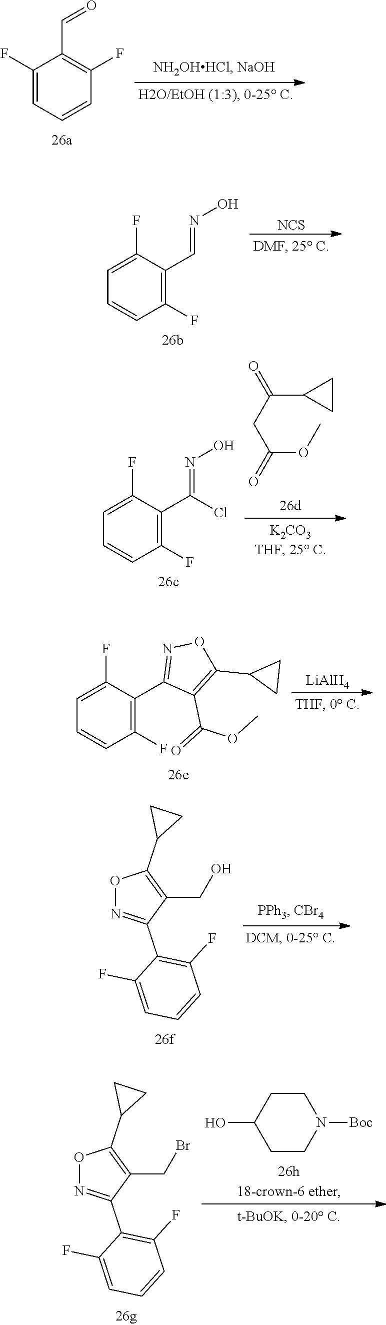

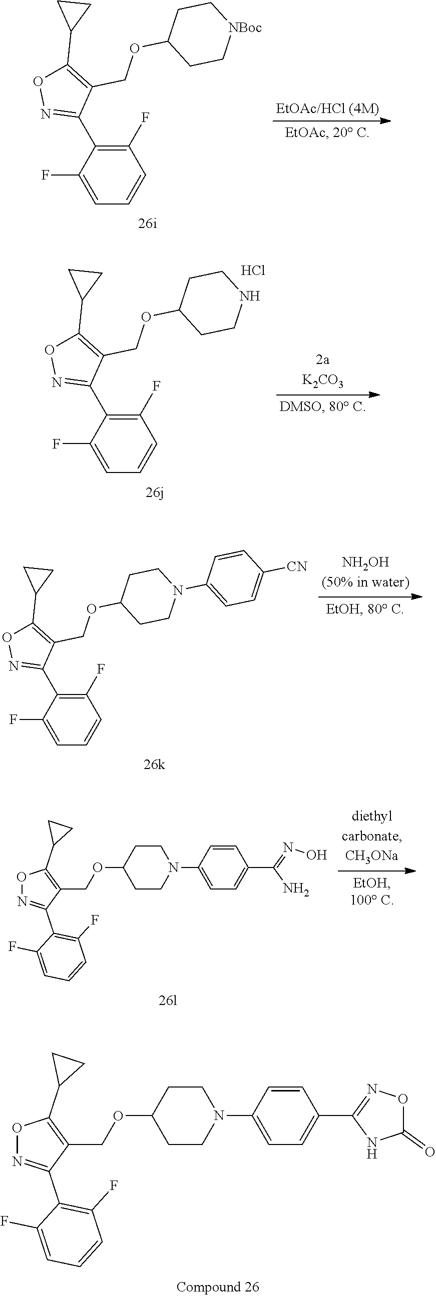 [SMILES: CC(C)(C)OC(=O)N1CCC(OCc2c(-c3c(F)cccc3F)noc2C2CC2)CC1 ; Fc1cccc(F)c1-c1noc(C2CC2)c1COC1CCNCC1 ; Cl ; N#Cc1ccc(N2CCC(OCc3c(-c4c(F)cccc4F)noc3C3CC3)CC2)cc1 ; N/C(=N\O)c1ccc(N2CCC(OCc3c(-c4c(F)cccc4F)noc3C3CC3)CC2)cc1 ; O=c1[nH]c(-c2ccc(N3CCC(OCc4c(-c5c(F)cccc5F)noc4C4CC4)CC3)cc2)no1]

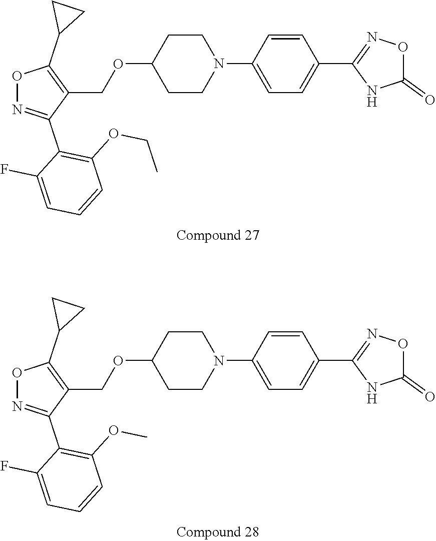 [SMILES: CCOc1cccc(F)c1-c1noc(C2CC2)c1COC1CCN(c2ccc(-c3noc(=O)[nH]3)cc2)CC1 ; COc1cccc(F)c1-c1noc(C2CC2)c1COC1CCN(c2ccc(-c3noc(=O)[nH]3)cc2)CC1]

(E)-2,6-difluorobenzaldehyde oxime (26b). A solution of hydroxylamine hydrochloride (733.53 mg, 10.56 mmol) and NaOH (422.20 mg, 10.56 mmol) in H₂O (3 mL) was added to a mixture of 2,6-difluorobenzaldehyde (26a) (1 g, 7.04 mmol) in ethanol (10 mL) at 0° C. The mixture was stirred at 25° C. for 16 hours The reaction mixture was poured into H₂O (10 mL) and extracted with ethyl acetate (20 mL*3). The combined organic layer was washed with brine (10 mL), dried over anhydrous Na₂SO₄, filtered and concentrated under reduced pressure to give 26b. MS mass calculated for [M+H]⁺(C₇H₅F₂NO) requires m/z, 158.0, LCMS found m/z, 158.0; ¹H NMR (400 MHz, CHLOROFORM-d) δ=9.75 (s, 1H), 8.35 (s, 1H), 7.39-7.29 (m, 1H), 6.98 (t, J=8.6 Hz, 2H).

(Z)-2,6-difluoro-N-hydroxybenzimidoyl chloride (26c). To a solution of (E)-2,6-difluorobenzaldehyde oxime (26b) (800 mg, 5.09 mmol) in DMF (8 mL) was added NCS (747.91 mg, 5.60 mmol) at 25° C. and the mixture was stirred at 25° C. for 16 hours. TLC showed the starting material was consumed completely and one new spot was detected. The reaction mixture of 26c (975 mg, crude) was used directly to the next step.

Methyl 5-cyclopropyl-3-(2,6-difluorophenyl)isoxazole-4-carboxylate (26e). To a solution of methyl 3-cyclopropyl-3-oxopropanoate (26d) (795.87 mg, 5.60 mmol) in THE (20 mL) was added K₂CO₃ (773.77 mg, 5.60 mmol). (Z)-2,6-difluoro-N-hydroxybenzimidoyl chloride (26c) (975 mg, 5.09 mmol) in DMF (8 mL) was added dropwise at 25° C. and the mixture was stirred at 25° C. for 2 hours. The reaction mixture was poured into H₂O (15 mL) and extracted with ethyl acetate (30 mL*2). The combined organic layer was washed with brine (10 mL), dried over anhydrous Na₂SO₄, filtered and concentrated under reduced pressure to give a residue. The residue was purified by column chromatography (SiO₂, petroleum ether:ethyl acetate=50:1 to 20:1) to give 26e. MS mass calculated for [M+H]⁺(C₁₄H₁₁F₂NO₃) requires m/z, 280.0, LCMS found m/z, 280.0; ¹H NMR (400 MHz, METHANOL-d4) δ=7.55 (tt, J=6.5, 8.5 Hz, 1H), 7.14-7.06 (m, 2H), 3.70 (s, 3H), 2.96-2.87 (m, 1H), 1.31 (s, 2H), 1.31-1.29 (m, 2H).

(5-cyclopropyl-3-(2,6-difluorophenyl)isoxazol-4-yl)methanol (26f). A suspension of LiAlH₄ (289.51 mg, 7.63 mmol) in THE (10 mL) was cooled to 0° C., and then a solution of methyl 5-cyclopropyl-3-(2,6-difluorophenyl)isoxazole-4-carboxylate (26e) (710 mg, 2.54 mmol) in THE (10 mL) was added dropwise at 0° C. The resulting solution was stirred at 0° C. for 30 minutes. The reaction mixture was quenched by the addition of ethyl acetate (10 mL), followed by water (5 mL), and then dried over Na₂SO₄. The reaction mixture was filtered, and the filtrate was concentrated under reduced pressure to give a residue. The residue was purified by column chromatography (SiO₂, petroleum ether:ethyl acetate=50:1 to 5:1) to give 26f. ¹H NMR (400 MHz, CHLOROFORM-d) δ=7.50-7.40 (m, 1H), 7.05 (t, J=7.9 Hz, 2H), 4.50 (s, 2H), 2.25-2.16 (m, 1H), 1.29-1.24 (m, 2H), 1.18-1.11 (m, 2H).

4-(bromomethyl)-5-cyclopropyl-3-(2,6-difluorophenyl)isoxazole (26g). To a solution of (5-cyclopropyl-3-(2,6-difluorophenyl)isoxazol-4-yl)methanol (26f) (518 mg, 2.06 mmol) in dichloromethane (20 mL) was added CBr₄ (1.03 g, 3.09 mmol) at 25° C., the mixture was cooled in an ice bath (about 0-5° C.), and then PPh₃ (1.08 g, 4.12 mmol) was added. The mixture was stirred at 25° C. for 2 hours. The reaction mixture was poured into H₂O (10 mL) and extracted with dichloromethane (20 mL*2). The combined organic layers were washed with brine (10 mL), dried over anhydrous Na₂SO₄, filtered and concentrated under reduced pressure to give a residue. The residue was purified by column chromatography (SiO₂, petroleum ether:ethyl acetate=50:1 to 10:1) to give 26g. ¹H NMR (400 MHz, CHLOROFORM-d) δ=7.54-7.42 (m, 1H), 7.06 (br t, J=7.9 Hz, 2H), 4.32 (s, 2H), 2.18-2.08 (m, 1H), 1.32-1.25 (m, 2H), 1.24-1.15 (m, 2H).

Tert-butyl 4-((5-cyclopropyl-3-(2,6-difluorophenyl)isoxazol-4-yl)methoxy)piperidine-1-carboxylate (26i). To a solution of tert-butyl 4-hydroxypiperidine-1-carboxylate (26h) (333.17 mg, 1.66 mmol) in THF (10 mL) was added 18-CROWN-6 (656.32 mg, 2.48 mmol) and t-BuOK (1 M solution in THF, 2.48 mL) at 0° C., after stirring at 0° C. for 30 minutes, 4-(bromomethyl)-5-cyclopropyl-3-(2,6-difluorophenyl)isoxazole (26g) (520 mg, 1.66 mmol) was added at 0° C. The mixture was stirred at 20° C. for 16 hours and was poured into H₂O (10 mL) and extracted with ethyl acetate (20 mL*2). The combined organic layer was washed with brine (10 mL), dried over anhydrous Na₂SO₄, filtered and concentrated under reduced pressure to give a residue. The residue was purified by column chromatography (SiO₂, petroleum ether:ethyl acetate=50:1 to 10:1) to give 26i. MS mass calculated for [M+H]⁺(C₂₃H₂₈F₂N₂O₄) requires m/z, 435.2, LCMS found m/z, 435.1; ¹H NMR (400 MHz, CHLOROFORM-d) δ=7.43 (tt, J=6.4, 8.5 Hz, 1H), 7.06-6.98 (m, 2H), 4.37 (s, 2H), 3.63-3.52 (m, 2H), 3.37 (tt, J=3.7, 7.9 Hz, 1H), 3.01 (ddd, J=3.5, 9.1, 13.2 Hz, 2H), 2.14 (tt, J=5.1, 8.5 Hz, 1H), 1.69-1.58 (m, 2H), 1.44 (s, 9H), 1.36 (dtd, J=3.9, 8.5, 12.8 Hz, 2H), 1.26-1.22 (m, 2H), 1.14-1.08 (m, 2H).

5-cyclopropyl-3-(2,6-difluorophenyl)-4-((piperidin-4-yloxy)methyl)isoxazole (26j). To a solution of tert-butyl 4-((5-cyclopropyl-3-(2,6-difluorophenyl)isoxazol-4-yl)methoxy)piperidine-1-carboxylate (26i) (560 mg, 1.29 mmol) in ethyl acetate (5 mL) was added HCl/ethyl acetate (4 M, 10 mL) at 20° C., and the mixture was stirred at 20° C. for 1 hour. The reaction mixture was concentrated under reduced pressure to give 26j. MS mass calculated for [M+H]⁺(C₁₈H₂₀F₂N₂O₂) requires m/z, 335.2, LCMS found m/z, 335.1.

4-(4-((5-cyclopropyl-3-(2,6-difluorophenyl)isoxazol-4-yl)methoxy)piperidin-1-yl)benzonitrile (26k). To a solution of 5-cyclopropyl-3-(2,6-difluorophenyl)-4-((piperidin-4-yloxy)methyl)isoxazole (26j) (150 mg, 404.51 umol, HCl) in DMSO (5 mL) was added K₂CO₃ (279.53 mg, 2.02 mmol) and 4-fluorobenzonitrile 2a (293.94 mg, 2.43 mmol) at 25° C., and the mixture was heated to 80° C. for 16 hours. The reaction mixture was poured into H₂O (10 mL) and extracted with ethyl acetate (20 mL*2). The combined organic layer was washed with brine (10 mL), dried over anhydrous Na₂SO₄, filtered and concentrated under reduced pressure to give a residue. The residue was purified by prep-TLC (SiO₂, petroleum ether:ethyl acetate=2:1) to give 26k. MS mass calculated for [M+H]⁺(C₂₅H₂₃F₂N₃O₂) requires m/z, 436.2, LCMS found m/z, 436.1; ¹H NMR (400 MHz, CHLOROFORM-d) δ=7.46 (d, J=9.0 Hz, 2H), 7.44-7.37 (m, 1H), 7.05-6.97 (m, 2H), 6.80 (d, J=9.0 Hz, 2H), 4.41 (s, 2H), 3.52-3.46 (m, 1H), 3.46-3.38 (m, 2H), 3.11-3.02 (m, 2H), 2.14 (tt, J=5.2, 8.4 Hz, 1H), 1.83-1.73 (m, 2H), 1.57-1.50 (m, 2H), 1.27-1.22 (m, 2H), 1.15-1.08 (m, 2H).

(Z)-4-(4-((5-cyclopropyl-3-(2,6-difluorophenyl)isoxazol-4-yl)methoxy)piperidin-1-yl)-N′-hydroxybenzimidamide (261). To a solution of 4-(4-((5-cyclopropyl-3-(2,6-difluorophenyl)isoxazol-4-yl)methoxy)piperidin-1-yl)-benzonitrile (26K) (140 mg, 321.50 umol) in ethanol (1.5 mL) was added hydroxylamine (21.24 mg, 321.50 umol, 50% in water) at 25° C., and the mixture was heated to 80° C. for 16 hours. The reaction mixture was filtered, and the filtrate was poured into H₂O (10 mL) and extracted with ethyl acetate (20 mL*2). The combined organic layer was washed with brine (10 mL), dried over Na₂SO₄, filtered and concentrated under reduced pressure to give a residue. The residue was purified by prep-TLC (SiO₂, dichloromethane:methanol=10:1) to give 261. MS mass calculated for [M+H]⁺(C₂₅H₂₆F₂N₄O₃) requires m/z, 469.2, LCMS found m/z, 469.1; ¹H NMR (400 MHz, CHLOROFORM-d) δ=7.49 (d, J=8.8 Hz, 2H), 7.45-7.36 (m, 1H), 7.05-6.97 (m, 2H), 6.86 (d, J=8.8 Hz, 2H), 4.81 (br s, 2H), 4.41 (s, 2H), 3.46-3.34 (m, 3H), 2.92 (ddd, J=3.2, 9.1, 12.4 Hz, 2H), 2.20-2.11 (m, 1H), 1.85-1.76 (m, 2H), 1.62-1.51 (m, 2H), 1.28-1.22 (m, 2H), 1.15-1.09 (m, 2H).

3-(4-(4-((5-cyclopropyl-3-(2,6-difluorophenyl)isoxazol-4-yl)methoxy)piperidin-1-yl)phenyl)-1,2,4-oxadiazol-5(4H)-one (Compound 26). To a solution of (Z)-4-(4-((5-cyclopropyl-3-(2,6-difluorophenyl)isoxazol-4-yl)methoxy)piperidin-1-yl)-N′-hydroxybenzimidamide (261) (55 mg, 117.40 umol) in ethanol (5 mL) was added diethyl carbonate (1.95 g, 16.51 mmol) and CH₃ONa (105.70 mg, 586.99 umol, 30% in MeOH) at 20° C. in a sealed tube. The mixture was heated to 100° C. for 0.5 hour. The reaction mixture was poured into H₂O (10 mL) and extracted with ethyl acetate (20 mL*2). The combined organic layer was washed with brine (10 mL), dried over anhydrous Na₂SO₄, filtered and concentrated under reduced pressure to give a residue. The residue was purified by prep-TLC (SiO₂, dichloromethane:methanol=10:1) to give Compound 26: MS mass calculated for [M+H]⁺(C₂₆H₂₄F₂N₄O₄) requires m/z, 495.1, LCMS found m/z, 541.2; ¹H NMR (400 MHz, CHLOROFORM-d) δ=10.91 (br s, 1H), 7.53 (d, J=8.8 Hz, 2H), 7.41-7.29 (m, 1H), 6.94 (t, J=7.8 Hz, 2H), 6.82 (br d, J=8.8 Hz, 2H), 4.34 (s, 2H), 3.46-3.31 (m, 3H), 3.06-2.94 (m, 2H), 2.13-2.02 (m, 1H), 1.73 (br dd, J=3.7, 12.6 Hz, 2H), 1.50-1.43 (m, 2H), 1.18 (br d, J=5.0 Hz, 2H), 1.10-1.00 (m, 2H).

3-(4-(4-((5-cyclopropyl-3-(2-ethoxy-6-fluorophenyl)isoxazol-4-yl)methoxy)piperidin-1-yl)phenyl)-1,2,4-oxadiazol-5(4H)-one (Compound 27) & 3-(4-(4-((5-cyclopropyl-3-(2-fluoro-6-methoxyphenyl)isoxazol-4-yl)methoxy)piperidin-1-yl)phenyl)-1,2,4-oxadiazol-5(4H)-one (Compound 28). To a solution of (Z)-4-(4-((5-cyclopropyl-3-(2,6-difluorophenyl)isoxazol-4-yl)methoxy)piperidin-1-yl)-N′-hydroxybenzimidamide (261) (80 mg, 170.76 umol) in ethanol (2 mL) was added diethyl carbonate (1.95 g, 16.51 mmol) and CH₃ONa (246.00 mg, 1.37 mmol, 30% in MeOH) at 20° C. in a sealed tube, and the mixture was heated to 100° C. for 16 hours. The reaction mixture was poured into H₂O (10 mL) and extracted with ethyl acetate (20 mL*2). The combined organic layer was washed with brine (10 mL), dried over anhydrous Na₂SO₄, filtered and concentrated under reduced pressure to give a residue. The residue was purified by prep-HPLC (column: Waters Xbridge BEH C18 100*30 mm*10 um; mobile phase: [water (10 Mm NH₄HCO₃)-ACN]; B %: 25%-40%, 8 min) to give Compound 27: MS mass calculated for [M+H]⁺(C₂₈H₂₉FN₄O₅) requires m/z, 521.2, LCMS found m/z, 521.3; ¹H NMR (400 MHz, CHLOROFORM-d) δ=7.59 (br d, J=8.2 Hz, 2H), 7.34 (q, J=7.4 Hz, 1H), 6.89 (br d, J=8.2 Hz, 2H), 6.82-6.72 (m, 2H), 4.37 (s, 2H), 4.05 (q, J=6.8 Hz, 2H), 3.44 (br s, 3H), 3.03 (br t, J=9.4 Hz, 2H), 2.22-2.12 (m, 1H), 1.74 (br s, 2H), 1.54 (br s, 2H), 1.32 (br t, J=6.7 Hz, 3H), 1.26 (br s, 2H), 1.10 (br d, J=8.2 Hz, 2H) and Compound 28: MS mass calculated for [M+H]⁺(C₂₇H₂₇FN₄O₅) requires m/z, 507.2, LCMS found m/z, 507.2; ¹H NMR (400 MHz, CHLOROFORM-d) δ=7.59 (d, J=8.8 Hz, 2H), 7.41-7.33 (m, 1H), 6.89 (d, J=9.0 Hz, 2H), 6.83-6.75 (m, 2H), 4.36 (s, 2H), 3.81 (s, 3H), 3.50-3.39 (m, 3H), 3.09-3.00 (m, 2H), 2.20-2.12 (m, 1H), 1.81-1.72 (m, 2H), 1.57 (td, J=3.8, 8.0 Hz, 2H), 1.26 (dd, J=2.2, 4.9 Hz, 2H), 1.14-1.07 (m, 2H).

### Example 29

### 3-(2-(4-((5-cyclopropyl-3-(2,6-dichlorophenyl)isoxazol-4-yl)methoxy)piperidin-1-yl)oxazol-4-yl)-1,2,4-oxadiazol-5(4H)-one

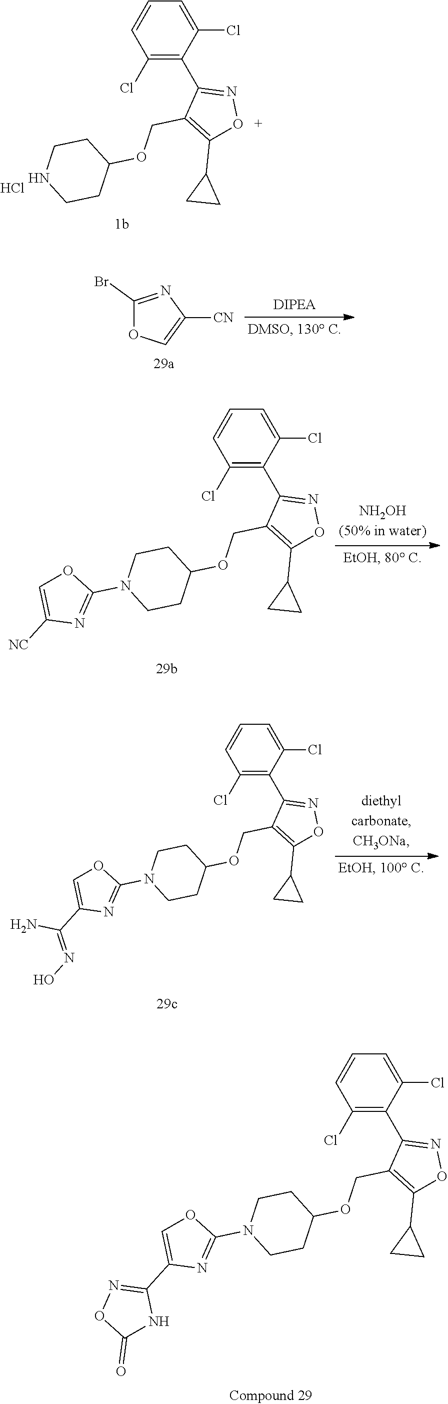

2-(4-((5-cyclopropyl-3-(2,6-dichlorophenyl)isoxazol-4-yl)methoxy)piperidin-1-yl)oxazole-4-carbonitrile (29b). To a solution of 5-cyclopropyl-3-(2,6-dichlorophenyl)-4-((piperidin-4-yloxy)methyl)isoxazole hydrochloride (1b) (150 mg, 371.54 umol) in DMSO (4 mL) was added DIPEA (144.05 mg, 1.11 mmol, 194.14 uL) and 2-bromooxazole-4-carbonitrile (29a) (64.26 mg, 371.54 umol), then heated to 130° C. and stirred for 18 hours. The reaction mixture was diluted with water (5 mL), extracted with ethyl acetate (10 mL*2), the organic phase was washed with brine (5 mL*5), dried over anhydrous Na₂SO₄, filtered and concentrated. The residue was purified by prep-TLC (SiO₂, petroleum ether:ethyl acetate=1:1) to afford 29b. MS mass calculated for [M+H]⁺(C₂₂H₂OCl₂N₄O₃) requires m/z, 459.1/461.1, LCMS found m/z, 459.0, 461.0; ¹H NMR (400 MHz, CHLOROFORM-d) δ=7.66 (s, 1H), 7.47-7.38 (m, 2H), 7.37-7.31 (m, 1H), 4.34 (s, 2H), 3.59-3.40 (m, 3H), 3.35-3.20 (m, 2H), 2.22-2.06 (m, 1H), 1.79-1.63 (m, 2H), 1.57-1.45 (m, 2H), 1.31-1.24 (m, 2H), 1.20-1.08 (m, 2H).

(Z)-2-(4-((5-cyclopropyl-3-(2,6-dichlorophenyl)isoxazol-4-yl)methoxy)piperidin-1-yl)-N′-hydroxyoxazole-4-carboximidamide (29c). To a solution of 2-(4-((5-cyclopropyl-3-(2,6-dichlorophenyl)isoxazol-4-yl)methoxy)piperidin-1-yl)oxazole-4-carbonitrile (29b) (100 mg, 217.71 umol) in ethanol (2 mL) was added hydroxylamine (2 mL, 50% in water) and stirred for 18 hours at 80° C. The reaction mixture was diluted with water (8 mL) and extracted with dichloromethane (10 mL*2). The combined organic phase was washed with brine (10 mL), dried over anhydrous Na₂SO₄, filtered and concentrated. The residue was purified by prep-TLC (SiO₂, dichloromethane:methanol=10:1) to afford 29c. MS mass calculated for [M+H]⁺(C₂₂H₂₃Cl₂N₅O₄) requires m/z, 492.1/494.1, LCMS found m/z, 492.1/494.1; ¹H NMR (400 MHz, CHLOROFORM-d) δ=7.48 (s, 1H), 7.44-7.38 (m, 2H), 7.36-7.29 (m, 1H), 5.08 (br s, 2H), 4.34 (s, 2H), 3.61-3.49 (m, 2H), 3.46 (td, J=3.8, 7.1 Hz, 1H), 3.22 (ddd, J=3.7, 8.2, 12.6 Hz, 2H), 2.20-2.09 (m, 1H), 1.77-1.67 (m, 2H), 1.56-1.44 (m, 2H), 1.34-1.23 (m, 2H), 1.19-1.06 (m, 2H).

3-(2-(4-((5-cyclopropyl-3-(2,6-dichlorophenyl)isoxazol-4-yl)methoxy)piperidin-1-yl)oxazol-4-yl)-1,2,4-oxadiazol-5(4H)-one (Compound 29). To a solution of (Z)-2-(4-((5-cyclopropyl-3-(2,6-dichlorophenyl)isoxazol-4-yl)methoxy)piperidin-1-yl)-N′-hydroxyoxazole-4-carboximidamide (29c) (50 mg, 101.55 umol) in ethanol (1 mL) was added diethyl carbonate (719.79 mg, 6.09 mmol, 738.25 uL) and NaOMe (109.73 mg, 609.32 umol, 30% in MeOH) in a sealed tube and heated to 100° C. and stirred for 1 hr. The reaction mixture was dried in vacuum to remove ethanol and diluted with water (10 mL). The mixture was extracted with ethyl acetate (20 mL*2) and the combined organic phase was washed with brine (10 mL), dried over Na₂SO₄, filtered and concentrated. The residue was purified by prep-HPLC (neutral condition; column: Waters Xbridge BEH C18 100*30 mm*10 um; mobile phase: [water (10 mM NH₄HCO₃)-ACN]; B %: 23%-53%, 8 min) to give Compound 29. MS mass calculated for [M+H]⁺(C₂₃H₂₁Cl₂N₅O₅) requires m/z, 518.1/520.1, LCMS found m/z, 518.1/520.1; ¹H NMR (400 MHz, CHLOROFORM-d) δ=7.77 (s, 1H), 7.47-7.39 (m, 2H), 7.38-7.30 (m, 1H), 4.35 (s, 2H), 3.59-3.45 (m, 3H), 3.33-3.24 (m, 2H), 2.19-2.08 (m, 1H), 1.79-1.66 (m, 2H), 1.60-1.46 (m, 2H), 1.33-1.24 (m, 2H), 1.19-1.09 (m, 2H).

### Example 30

### 3-(4-(4-((5-cyclopropyl-3-(2,6-dichlorophenyl)isoxazol-4-yl)methoxy)piperidin-1-yl)thiophen-2-yl)-1,2,4-oxadiazol-5(4H)-one

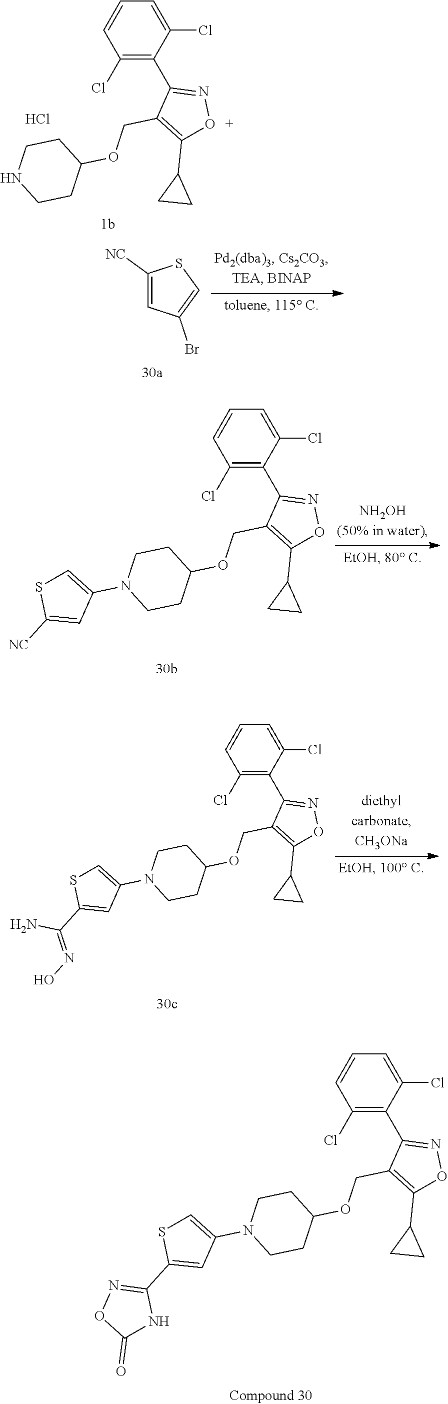

4-(4-((5-cyclopropyl-3-(2,6-dichlorophenyl)isoxazol-4-yl)methoxy)piperidin-1-yl)thiophene-2-carbonitrile (30b). To a mixture of 5-cyclopropyl-3-(2,6-dichlorophenyl)-4-((piperidin-4-yloxy)methyl)isoxazole hydrochloride (1b) (200 mg, 495.38 umol) and 4-bromothiophene-2-carbonitrile (30a) (102.47 mg, 544.92 umol) in toluene (5 mL) was added Pd₂(dba)₃ (45.36 mg, 49.54 umol), BINAP (370.15 mg, 594.46 umol), Cs₂CO₃ (807.02 mg, 2.48 mmol) and TEA (100.25 mg, 990.76 umol, 137.90 uL) under N₂. The mixture was degassed and purged with N₂ 3 times, and heated to reflux for 18 hours. The reaction mixture was diluted with ethyl acetate (10 mL) and added 500 mg 3-mercaptopropyl-functionalized silica gel, the suspension was stirred for 1 hour at 45° C. and filtered through a Celite pad. The filtrate was concentrated under reduced pressure. The residue was diluted with water (10 mL) and extracted with ethyl acetate (15 mL*2). The combined organic phase was washed with brine (10 mL), dried over anhydrous Na₂SO₄, filtered and concentrated. The residue was purified by column chromatography (SiO₂, petroleum ether:ethyl acetate=20:1 to 10:1) to give 30b. ¹H NMR (400 MHz, CHLOROFORM-d) δ=7.41-7.35 (m, 2H), 7.32-7.28 (m, 1H), 7.26 (d, J=2.0 Hz, 1H), 6.33 (d, J=1.8 Hz, 1H), 4.34 (s, 2H), 3.41 (td, J=3.7, 7.5 Hz, 1H), 3.15-3.04 (m, 2H), 2.88-2.74 (m, 2H), 2.19-2.10 (m, 1H), 1.85-1.71 (m, 2H), 1.64-1.55 (m, 2H), 1.31-1.26 (m, 2H), 1.16-1.07 (m, 2H).

(Z)-4-(4-((5-cyclopropyl-3-(2,6-dichlorophenyl)isoxazol-4-yl)methoxy)piperidin-1-yl)-N′-hydroxythiophene-2-carboximidamide (30c). To a solution of 4-(4-((5-cyclopropyl-3-(2,6-dichlorophenyl)isoxazol-4-yl)methoxy)piperidin-1-yl)thiophene-2-carbonitrile (30b) (25 mg, 52.70 umol) in ethanol (0.5 mL) was added hydroxylamine (0.2 mL, 50% in water). The mixture was heated to 80° C. and stirred for 1 hour. The reaction mixture was dried in vacuum, diluted with water (5 mL) and extracted with ethyl acetate (10 mL*2). The combined organic phase was washed with brine (10 mL), dried over anhydrous Na₂SO₄, filtered and concentrated. The residue was purified by prep-TLC (SiO₂, dichloromethane:methanol=10:1) to give 30c. MS mass calculated for [M+H]⁺(C₂₃H₂₄Cl₂N₄O₃S) requires m/z, 507.1/509.1, LCMS found m/z, 507.1/508.9.

3-(4-(4-((5-cyclopropyl-3-(2,6-dichlorophenyl)isoxazol-4-yl)methoxy)piperidin-1-yl)thiophen-2-yl)-1,2,4-oxadiazol-5(4H)-one (Compound 30). To a solution of (Z)-4-(4-((5-cyclopropyl-3-(2,6-dichlorophenyl)isoxazol-4-yl)methoxy)piperidin-1-yl)-N′-hydroxythiophene-2-carboximidamide (30c) (20 mg, 39.41 umol) in ethanol (0.5 mL) was added diethyl carbonate (279.36 mg, 2.36 mmol, 286.52 uL) and NaOMe (35.49 mg, 197.07 umol, 30% in MeOH) in a sealed tube. The reaction mixture was heated to 100° C. and stirred for 2 hours and was concentrated under reduced pressure. The residue was diluted with water (5 mL) and extracted with ethyl acetate (10 mL*2). The combined organic phase was washed with brine (10 mL), dried over Na₂SO₄, filtered and concentrated. The residue was purified by prep-HPLC (TFA condition; column: Phenomenex Synergi C18 150*25*10 um; mobile phase: [water (0.1% TFA)-ACN]; B %: 35%-65%, 10 min) to give Compound 30. MS mass calculated for [M+H]⁺(C₂₄H₂₂Cl₂N₄O₄S) requires m/z, 533.1/535.1, LCMS found m/z, 533.1/535.1; ¹H NMR (400 MHz, CHLOROFORM-d) δ=7.49-7.34 (m, 3H), 7.33-7.27 (m, 1H), 6.46 (s, 1H), 4.35 (s, 2H), 3.45 (br s, 1H), 3.16 (br t, J=7.7 Hz, 2H), 2.90 (br t, J=7.9 Hz, 2H), 2.20-2.09 (m, 1H), 1.84 (br s, 2H), 1.64 (br d, J=8.8 Hz, 2H), 1.32-1.22 (m, 2H), 1.18-1.09 (m, 2H).

### Example 31. 3-(4-((3R,4S)-4-((5-cyclopropyl-3-(2,6-dichlorophenyl)isoxazol-4-yl)methoxy)-3-methylpiperidin-1-yl)phenyl)-1,2,4-oxadiazol-5(4H)-one

### Example 32. 3-(4-((3S,4R)-4-((5-cyclopropyl-3-(2,6-dichlorophenyl)isoxazol-4-yl)methoxy)-3-methylpiperidin-1-yl)phenyl)-1,2,4-oxadiazol-5(4H)-one Example 33. 3-(4-((3S,4S)-4-((5-cyclopropyl-3-(2,6-dichlorophenyl)isoxazol-4-yl)methoxy)-3-methylpiperidin-1-yl)phenyl)-1,2,4-oxadiazol-5(4H)-one Example 34. 3-(4-((3R,4R)-4-((5-cyclopropyl-3-(2,6-dichlorophenyl)isoxazol-4-yl)methoxy)-3-methylpiperidin-1-yl)phenyl)-1,2,4-oxadiazol-5(4H)-one

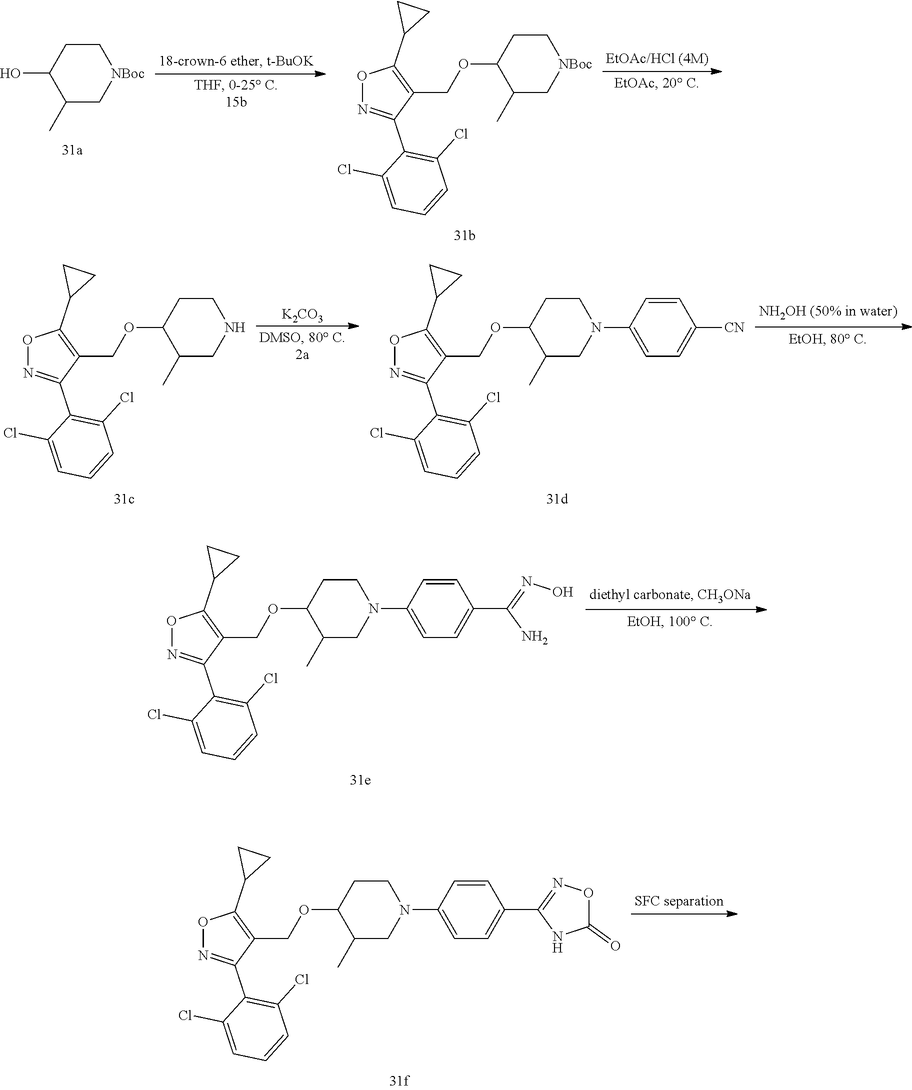

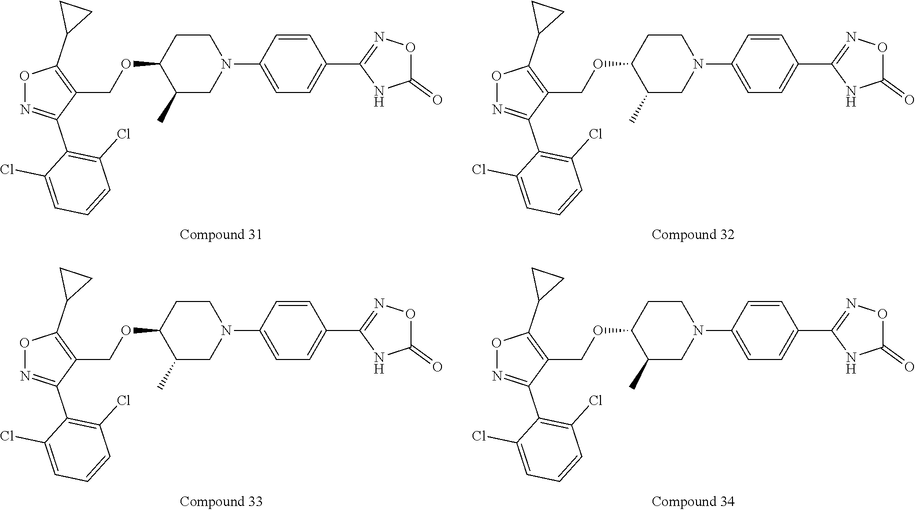 [SMILES: C[C@@H]1CN(c2ccc(-c3noc(=O)[nH]3)cc2)CC[C@@H]1OCc1c(-c2c(Cl)cccc2Cl)noc1C1CC1 ; C[C@H]1CN(c2ccc(-c3noc(=O)[nH]3)cc2)CC[C@H]1OCc1c(-c2c(Cl)cccc2Cl)noc1C1CC1 ; C[C@H]1CN(c2ccc(-c3noc(=O)[nH]3)cc2)CC[C@@H]1OCc1c(-c2c(Cl)cccc2Cl)noc1C1CC1 ; C[C@@H]1CN(c2ccc(-c3noc(=O)[nH]3)cc2)CC[C@H]1OCc1c(-c2c(Cl)cccc2Cl)noc1C1CC1]

Tert-butyl 4-((5-cyclopropyl-3-(2,6-dichlorophenyl)isoxazol-4-yl)methoxy)-3-methylpiperidine-1-carboxylate (31b). To a solution of tert-butyl 4-hydroxy-3-methylpiperidine-1-carboxylate (31a) (600 mg, 2.79 mmol) in THF (10 mL) was added 18-CROWN-6 (1.10 g, 4.18 mmol) and t-BuOK (1 M solution in THF, 4.18 mL) at 0° C. The mixture was stirred at 25° C. for 30 minutes, then 4-(bromomethyl)-5-cyclopropyl-3-(2,6-dichlorophenyl)isoxazole (15b) (1.02 mg, 2.93 mmol) in THF (10 mL) was added dropwise at 25° C. The mixture was stirred at 25° C. for 16 hours and was poured into H₂O (10 mL) and extracted with ethyl acetate (30 mL*2). The combined organic layer was washed with brine (10 mL), dried over anhydrous Na₂SO₄, filtered and concentrated under reduced pressure to give a residue. The residue was purified by column chromatography (SiO₂, petroleum ether:ethyl acetate=50:1 to 3:1) to give 31b; MS mass calculated for [M+H]⁺(C₂₄H₃₀Cl₂N₂O₄) requires m/z, 481.2, LCMS found m/z, 481.2.

5-cyclopropyl-3-(2,6-dichlorophenyl)-4-(((3-methylpiperidin-4-yl)oxy)methyl)isoxazole (31b). To a solution of tert-butyl 4-((5-cyclopropyl-3-(2,6-dichlorophenyl)isoxazol-4-yl)methoxy)-3-methylpiperidine-1-carboxylate (31b) (1.34 g, 2.78 mmol) in ethyl acetate (5 mL) was added HCl/ethyl acetate (10 mL, 4 M) at 20° C. The mixture was stirred at 20° C. for 2 hours and was concentrated under reduced pressure to give 31b. MS mass calculated for [M+H]⁺(C₁₉H₂₂Cl₂N₂O₂) requires m/z, 381.1/383.1, LCMS found m/z, 381.0/383.0;

4-(4-((5-cyclopropyl-3-(2,6-dichlorophenyl)isoxazol-4-yl)methoxy)-3-methylpiperidin-1-yl)benzonitrile (31d). To a solution of 5-cyclopropyl-3-(2,6-dichlorophenyl)-4-(((3-methylpiperidin-4-yl)oxy)methyl)isoxazole (31c)(1.1 g, 2.63 mmol, HCl) in DMSO (15 mL) was added K₂CO₃ (1.82 g, 13.17 mmol) and 4-fluorobenzonitrile (2a) (2.00 g, 16.51 mmol) at 25° C. The mixture was heated to 80° C. for 16 hours and was poured into water (15 mL) and extracted with ethyl acetate (30 mL*2). The combined organic layer was washed with brine (10 mL), dried over anhydrous Na₂SO₄, filtered and concentrated under reduced pressure to give a residue. The residue was purified by column chromatography (SiO₂, petroleum ether:ethyl acetate=50:1 to 5:1) to give 31d. MS mass calculated for [M+H]⁺(C₂₆H₂₅Cl₂N₃O₂) requires m/z, 482.1/484.1, LCMS found m/z, 482.2/484.1.

(Z)-4-(4-((5-cyclopropyl-3-(2,6-dichlorophenyl)isoxazol-4-yl)methoxy)-3-methylpiperidin-1-yl)-N′-hydroxybenzimidamide (31e). To a solution of 4-(4-((5-cyclopropyl-3-(2,6-dichlorophenyl)isoxazol-4-yl)methoxy)-3-methylpiperidin-1-yl)benzonitrile (31d) (950 mg, 1.97 mmol) in ethanol (10 mL) was added hydroxylamine (10 mL, 50% in water,) at 25° C. The mixture was heated to 80° C. for 16 hours and was filtered. The filtrate was poured into H₂O (15 mL) and the mixture was extracted with ethyl acetate (30 mL*2). The combined organic layer was washed with brine (10 mL), dried over anhydrous Na₂SO₄, filtered and concentrated under reduced pressure to give a residue. The residue was purified by column chromatography (SiO₂, petroleum ether:ethyl acetate=50:1 to 1:1) to give 31e. MS mass calculated for [M+H]⁺(C₂₆H₂₈Cl₂N₄O₃) requires m/z, 515.2/517.2, LCMS found m/z, 515.1/517.1; ¹H NMR (400 MHz, CHLOROFORM-d) δ=7.49 (d, J=8.8 Hz, 3H), 7.44-7.39 (m, 2H), 7.39-7.30 (m, 2H), 7.27-7.23 (m, 1H), 6.88-6.79 (m, 3H), 4.80 (br s, 3H), 4.45 (d, J=12.1 Hz, 1H), 4.39 (d, J=11.7 Hz, 1H), 4.26-4.18 (m, 2H), 3.56 (br d, J=12.6 Hz, 1H), 3.50 (br d, J=12.6 Hz, 1H), 3.41 (br d, J=2.9 Hz, 1H), 3.19-3.09 (m, 1H), 2.99-2.89 (m, 1H), 2.89-2.77 (m, 1H), 2.77-2.68 (m, 1H), 2.47 (dd, J=10.3, 12.5 Hz, 1H), 2.24-2.09 (m, 2H), 1.91-1.80 (m, 2H), 1.78-1.66 (m, 2H), 1.60 (br d, J=9.5 Hz, 1H), 1.48-1.36 (m, 1H), 1.31-1.23 (m, 4H), 1.17-1.09 (m, 3H), 0.87 (d, J=6.6 Hz, 3H), 0.84 (d, J=6.8 Hz, 2H).

3-(4-(4-((5-cyclopropyl-3-(2,6-dichlorophenyl)isoxazol-4-yl)methoxy)-3-methylpiperidin-1-yl)phenyl)-1,2,4-oxadiazol-5(4H)-one (31f). To a solution of (Z)-4-(4-((5-cyclopropyl-3-(2,6-dichlorophenyl)isoxazol-4-yl)methoxy)-3-methylpiperidin-yl)-N′-hydroxybenzimidamide (31e) (850 mg, 1.65 mmol) in ethanol (10 mL) was added diethyl carbonate (4.88 g, 41.27 mmol, 5 mL) and CH₃ONa (2 Ml, 30% in MeOH) at 20° C. in a sealed tube and the mixture was heated to 100° C. for 16 hours. The reaction mixture was poured into H₂O (10 mL) and was extracted with ethyl acetate (20 mL*2). The combined organic layer was washed with brine (10 mL), dried over anhydrous Na₂SO₄, filtered and concentrated under reduced pressure to give a residue. The residue was purified by column chromatography (SiO₂, petroleum ether:ethyl acetate=50:1 to 0:1) to give 31f as a mixture of the four isomers which was separated by SFC (column: DAICEL CHIRALPAK IG (250 mm*30 mm, 10 um); mobile phase: [0.1% NH₃H₂O MeOH]; B %: 50%-50%) to give Compound 33 (one of the trans isomers): MS mass calculated for [M+H]⁺(C₂₇H₂₆Cl₂N₄O₄) requires m/z, 541.1/543.1, LCMS found m/z, 541.1/543.1; ¹H NMR (400 MHz, CHLOROFORM-d) δ=11.11 (br s, 1H), 7.61 (d, J=8.9 Hz, 2H), 7.46-7.39 (m, 2H), 7.37-7.31 (m, 1H), 6.89 (d, J=8.9 Hz, 2H), 4.45 (d, J=12.0 Hz, 1H), 4.23 (d, J=11.9 Hz, 1H), 3.67-3.54 (m, 2H), 3.01 (dt, J=3.9, 8.9 Hz, 1H), 2.91-2.81 (m, 1H), 2.61 (dd, J=10.0, 12.9 Hz, 1H), 2.22-2.13 (m, 1H), 1.90-1.81 (m, 1H), 1.76-1.65 (m, 1H), 1.46-1.35 (m, 1H), 1.32-1.27 (m, 2H), 1.18-1.11 (m, 2H), 0.88 (d, J=6.6 Hz, 3H).

Compound 34 (one of the trans isomers): MS mass calculated for [M+H]⁺(C₂₇H₂₆Cl₂N₄O₄) requires m/z, 541.1/543.1, LCMS found m/z, 541.1/543.1; ¹H NMR (400 MHz, CHLOROFORM-d) δ=7.60 (d, J=8.9 Hz, 2H), 7.46-7.39 (m, 2H), 7.37-7.31 (m, 1H), 6.89 (d, J=9.0 Hz, 2H), 4.45 (d, J=12.0 Hz, 1H), 4.23 (d, J=11.9 Hz, 1H), 3.67-3.53 (m, 2H), 3.01 (dt, J=4.1, 9.0 Hz, 1H), 2.91-2.81 (m, 1H), 2.61 (dd, J=9.9, 13.0 Hz, 1H), 2.22-2.13 (m, 1H), 1.91-1.81 (m, 1H), 1.76-1.64 (m, 1H), 1.47-1.34 (m, 1H), 1.32-1.26 (m, 2H), 1.19-1.11 (m, 2H), 0.88 (d, J=6.7 Hz, 3H).

A mixture of Compound 31 & Compound 32 (170 mg) was obtained in the first SFC separation and then re-purified by SFC (column: DAICEL CHIRALCEL OJ (250 mm*50 mm, 10 um); mobile phase: [0.1NH₃H₂O MEOH]; B %: 45%-45%) to give: Compound 31 (one of the cis isomers): MS mass calculated for [M+H]⁺(C₂₇H₂₆Cl₂N₄O₄) requires m/z, 541.1/543.1, LCMS found m/z, 541.1/543.1, LCMS found m/z, 541.1/543.1; ¹H NMR (400 MHz, CHLOROFORM-d) δ=7.60 (d, J=8.9 Hz, 2H), 7.42-7.34 (m, 2H), 7.30-7.27 (m, 1H), 6.88 (d, J=9.0 Hz, 2H), 4.40 (d, J=11.6 Hz, 1H), 4.25 (d, J=11.6 Hz, 1H), 3.47-3.40 (m, 1H), 3.32-3.20 (m, 2H), 3.02-2.85 (m, 2H), 2.14 (tt, J=5.1, 8.5 Hz, 1H), 1.91-1.72 (m, 2H), 1.62-1.52 (m, 1H), 1.32-1.24 (m, 2H), 1.18-1.11 (m, 2H), 0.84 (d, J=6.8 Hz, 3H).

Compound 32 (one of the cis isomers): MS mass calculated for [M+H]⁺(C₂₇H₂₆Cl₂N₄O₄) requires m/z, 541.1/543.1, LCMS found m/z, 541.1/543.1; ¹H NMR (400 MHz, CHLOROFORM-d) δ=7.57 (br d, J=8.2 Hz, 2H), 7.41-7.33 (m, 2H), 7.27-723 (m, 1H), 6.83 (br d, J=7.2 Hz, 2H), 4.39 (d, J=11.6 Hz, 1H), 4.24 (d, J=11.6 Hz, 1H), 3.42 (br s, 1H), 3.21 (br d, J=12.0 Hz, 2H), 2.99-2.80 (m, 2H), 2.18-2.09 (m, 1H), 1.89-1.69 (m, 2H), 1.55 (br t, J=12.0 Hz, 1H), 1.31-1.23 (m, 2H), 1.17-1.09 (m, 2H), 0.83 (br d, J=6.8 Hz, 3H).

### Example 35

### 3-(5-(4-((5-cyclopropyl-3-(2,6-dichlorophenyl)isoxazol-4-yl)methoxy)piperidin-1-yl)thiophen-2-yl)-1,2,4-oxadiazol-5(4H)-one

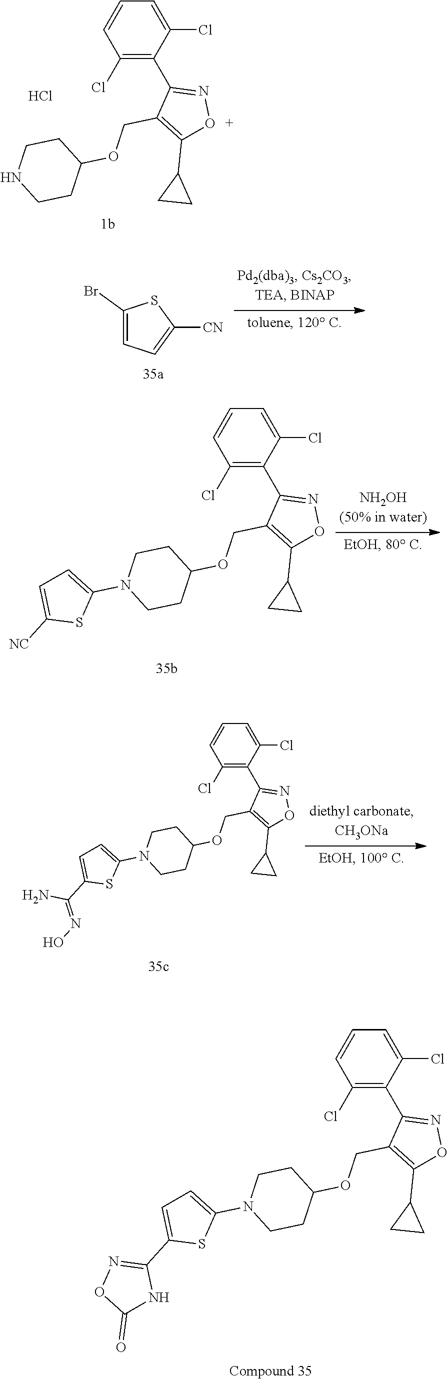

5-(4-((5-cyclopropyl-3-(2,6-dichlorophenyl)isoxazol-4-yl)methoxy)piperidin-1-yl)thiophene-2-carbonitrile (35b). To a solution of 5-cyclopropyl-3-(2,6-dichlorophenyl)-4-((piperidin-4-yloxy)methyl)isoxazole hydrochloride (1b) (483.07 mg, 1.20 mmol) and 5-bromothiophene-2-carbonitrile (35a) (150 mg, 797.68 umol) in toluene (20 mL) was added Cs₂CO₃ (1.56 g, 4.79 mmol), Pd₂(dba)₃ (73.05 mg, 79.77 umol), BINAP (596.03 mg, 957.22 umol), TEA (161.43 mg, 1.60 mmol, 222.05 uL) at 25° C. The suspension was degassed under vacuum and purged with N₂ several times. The mixture was heated to 120° C. and stirred for 16 hours under N₂. The mixture was filtered and the filter cake was washed by dichloromethane (50 mL). The combined filtrate was concentrated under reduced pressure. Water (5 mL) and ethyl acetate (5 mL) were added into the residue and separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by column chromatography (SiO₂, petroleum ether:ethyl acetate=1:0 to 0:1) to give 35b. MS mass calculated for [M+H]⁺(C₂₃H₂₁Cl₂N₃O₂S) requires m/z, 474.1/476.1, LCMS found m/z, 474.0/476.1; ¹H NMR (400 MHz, CHLOROFORM-d) δ=7.41-7.36 (m, 2H), 7.34-7.28 (m, 2H), 5.90 (d, J=4.3 Hz, 1H), 4.34 (s, 2H), 3.50 (tt, J=3.3, 6.6 Hz, 1H), 3.16 (ddd, J=3.9, 8.4, 12.2 Hz, 2H), 3.06-2.98 (m, 2H), 2.13 (tt, J=5.1, 8.5 Hz, 1H), 1.82-1.73 (m, 2H), 1.64 (qd, J=6.7, 10.8 Hz, 2H), 1.29-1.26 (m, 2H), 1.17-1.11 (m, 2H).

(Z)-5-(4-((5-cyclopropyl-3-(2,6-dichlorophenyl)isoxazol-4-yl)methoxy)piperidin-1-yl)-N′-hydroxythiophene-2-carboximidamide (35c). To a solution of 5-(4-((5-cyclopropyl-3-(2,6-dichlorophenyl)isoxazol-4-yl)methoxy)piperidin-1-yl)thiophene-2-carbonitrile (35b) (110 mg, 231.87 umol) in ethanol (6 mL) was added hydroxylamine (3 mL, 50% in water) at 25° C. The reaction was degassed and purged with N₂ 3 times and the mixture was stirred at 80° C. for 2 hours under N₂ atmosphere. The reaction mixture was concentrated under reduced pressure to remove solvent. Water (5 mL) and ethyl acetate (5 mL) were added into the residue and phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, dichloromethane:methanol=10:1) to give 35c. ¹H NMR (400 MHz, CHLOROFORM-d) δ=7.44-7.37 (m, 2H), 7.34-7.28 (m, 1H), 6.94 (br d, J=4.1 Hz, 1H), 6.65 (br s, 1H), 5.91 (br d, J=4.1 Hz, 1H), 4.74 (br s, 2H), 4.33 (s, 2H), 3.50-3.40 (m, 1H), 3.26-3.09 (m, 2H), 3.04-2.86 (m, 2H), 2.23-2.07 (m, 1H), 1.92-1.73 (m, 2H), 1.67-1.58 (m, 2H), 1.38-1.24 (m, 2H), 1.22-1.09 (m, 2H).

3-(5-(4-((5-cyclopropyl-3-(2,6-dichlorophenyl)isoxazol-4-yl)methoxy)piperidin-1-yl)thiophen-2-yl)-1,2,4-oxadiazol-5(4H)-one (Compound 35). To a mixture of (Z)-5-(4-((5-cyclopropyl-3-(2,6-dichlorophenyl)isoxazol-4-yl)methoxy)piperidin-1-yl)-N′-hydroxythiophene-2-carboximidamide (35c) (80 mg, 157.66 umol) in ethanol (4 mL) was added diethyl carbonate (1.95 g, 16.51 mmol, 2 mL) and CH₃ONa (0.5 mL, 30% in MeOH) at 20° C. in a sealed tube. The mixture was stirred at 100° C. for 10 hours and was concentrated under reduced pressure to remove solvent. The residue was diluted with ethyl acetate (5 mL) and water (5 mL) and the phases were separated. The aqueous phase was extracted with ethyl acetate (5 mL*4). The combined organic phase was washed with brine (20 mL*2), dried with anhydrous Na₂SO₄, filtered and concentrated under reduced pressure and purified by prep-HPLC (neutral condition: column: Waters Xbridge BEH C18 100*25 mm*5 um; mobile phase: [water(10 mM NH₄HCO₃)-ACN]; B %: 20%-50%, 10 min) to give Compound 35. MS mass calculated for [M+H]⁺(C₂₄H₂₂Cl₂N₄O₄S) requires m/z, 533.1/535.1, LCMS found m/z, 533.1/535.1; ¹H NMR (400 MHz, CHLOROFORM-d) δ=7.41-7.36 (m, 2H), 7.33-7.29 (m, 1H), 7.28 (br s, 1H), 7.26 (s, 1H), 5.98 (d, J=4.2 Hz, 1H), 4.34 (s, 2H), 3.50 (td, J=3.3, 6.6 Hz, 1H), 3.18 (ddd, J=3.7, 8.3, 12.2 Hz, 2H), 3.08-2.99 (m, 2H), 2.14 (tt, J=5.1, 8.5 Hz, 1H), 1.78 (dt, J=4.0, 8.6 Hz, 2H), 1.64 (qd, J=6.5, 10.9 Hz, 2H), 1.31-1.25 (m, 2H), 1.17-1.11 (m, 2H).

### Example 36

### 3-(4-(4-((5-cyclopropyl-3-(2-(trifluoromethoxy)phenyl)isoxazol-4-yl)methoxy)piperidin-1-yl)phenyl)-1,2,4-oxadiazol-5(4H)-one

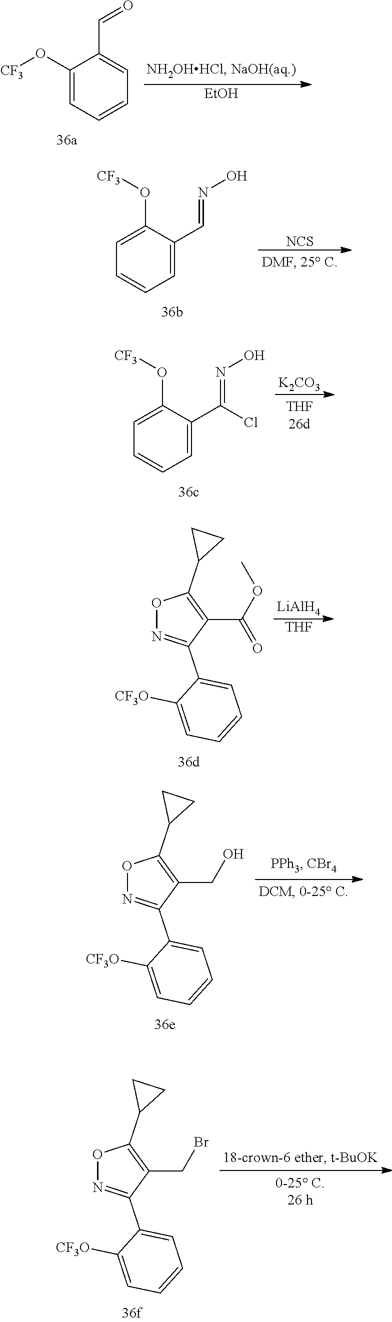

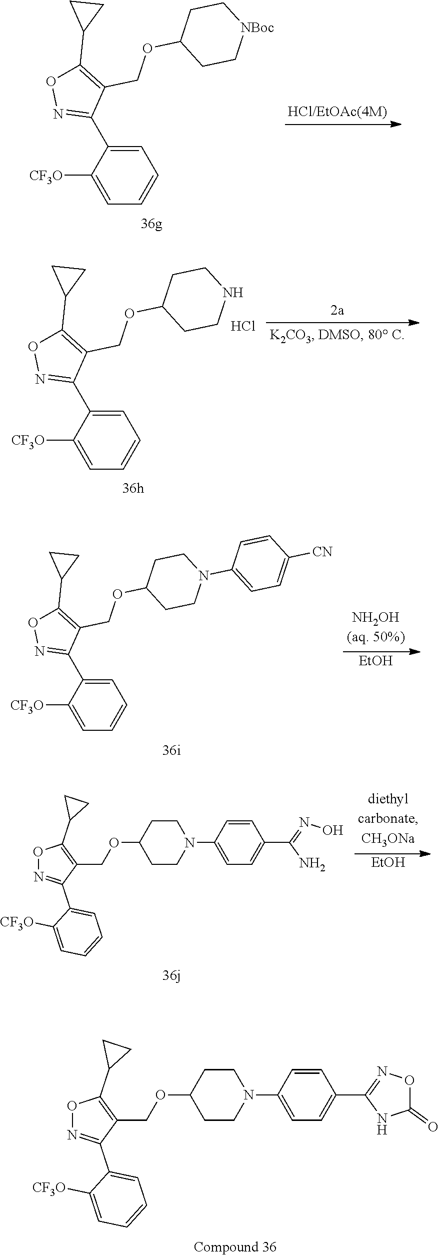

(E)-2-(trifluoromethoxy)benzaldehyde oxime (36b). A solution of hydroxylamine hydrochloride (402.06 mg, 5.79 mmol,) and NaOH (252.46 mg, 6.31 mmol) in water (5 mL) was added dropwise to a solution of 2-(trifluoromethoxy)benzaldehyde (36a) (1 g, 5.26 mmol) in ethanol (10 mL) at 20° C. The mixture was stirred at 35° C. for 6 hours and was concentrated to remove most of the ethanol. Water (10 mL) was added and extracted with ethyl acetate (15 mL*3). The combined organic layers were washed with brine (10 mL), dried over anhydrous Na₂SO₄ and concentrated under reduced pressure to give 36b. MS mass calculated for [M+H]⁺(C₈H₆F₃NO₂) requires m/z, 206.0 LCMS found m/z, 206.0; ¹H NMR (CHLOROFORM-d, 400 MHz): δ=8.43 (s, 1H), 7.90 (dd, J=7.7, 1.5 Hz, 1H), 7.74 (s, 1H), 7.40-7.47 (m, 1H), 7.28-7.36 (m, 2H).

(Z)—N-hydroxy-2-(trifluoromethoxy)benzimidoyl chloride (36c). To a solution of (E)-2-(trifluoromethoxy)benzaldehyde oxime (36b) (800 mg, 3.90 mmol) in DMF (8 mL) was added NCS (572.84 mg, 4.29 mmol) at 20° C. and stirred for 12 hours. 36c dissolved in DMF as colorless solution was used into next step directly. MS mass calculated for [M+H]⁺(C₈H₅ClF₃NO₂) requires m/z, 240.0/242.0, LCMS found m/z, 240.0/242.0.

Methyl 5-cyclopropyl-3-(2-(trifluoromethoxy)phenyl)isoxazole-4-carboxylate (36d). To a solution of methyl 3-cyclopropyl-3-oxopropanoate (26d) (503.15 mg, 3.54 mmol) and K₂CO₃ (489.19 mg, 3.54 mmol) in THE (10 mL) was added (Z)—N-hydroxy-2-(trifluoromethoxy)benzimidoyl chloride (36c) (800 mg, 3.34 mmol) in DMF (8 mL) dropwise at 20° C. The mixture was stirred at 20° C. for 12 hours and was concentrated to remove most of the solvents. Water (10 mL) was added to the residue and the mixture was extracted with ethyl acetate (20 mL*3). The combined organic layer was washed with brine (10 mL), dried over anhydrous Na₂SO₄ and concentrated under reduced pressure. The residue was purified by silica gel column chromatography to give 36d. MS mass calculated for [M+H]⁺(C₁₅H₁₂F₃NO₄) requires m/z, 328.1 LCMS found m/z, 328.0; ¹H NMR (METHANOL-d4, 400 MHz): δ=7.58-7.65 (m, 1H), 7.50-7.54 (m, 1H), 7.40-7.48 (m, 2H), 3.67 (s, 3H), 2.82-2.93 (m, 1H), 1.23-1.33 (m, 4H).

(5-cyclopropyl-3-(2-(trifluoromethoxy)phenyl)isoxazol-4-yl)methanol (36e). To a solution of methyl 5-cyclopropyl-3-(2-(trifluoromethoxy)phenyl)isoxazole-4-carboxylate (36d) (500 mg, 1.53 mmol) in THE (20 mL) was added LiAlH₄ (173.97 mg, 4.58 mmol) at 0° C. The mixture was stirred at 0° C. for 30 minutes, then was warmed to 15° C. for 1 hour. The reaction mixture was quenched dropwise addition of with excess ethyl acetate (20 mL) at 18° C. The resulting mixture was stirred at 20° C. for 30 minutes and filtered. The filtrate was concentrated under reduced pressure. The residue was purified by silica gel column chromatography to give 36e. MS mass calculated for [M+H]⁺(C₁₄H₁₂F₃NO₃) requires m/z, 300.1 LCMS found m/z, 300.0; ¹H NMR (CHLOROFORM-d, 400 MHz): δ=7.47-7.54 (m, 1H), 7.44 (dd, J=7.7, 2.0 Hz, 1H), 7.33 (t, J=7.1 Hz, 2H), 4.42 (s, 2H), 2.11 (tt, J=8.4, 5.1 Hz, 1H), 1.12-1.22 (m, 2H), 1.01-1.08 (m, 2H).

4-(bromomethyl)-5-cyclopropyl-3-(2-(trifluoromethoxy)phenyl)isoxazole (36f). To a solution of (5-cyclopropyl-3-(2-(trifluoromethoxy)phenyl)isoxazol-4-yl)methanol (36e) (200 mg, 668.35 umol) in dichloromethane (10 mL) was added PPh₃ (350.60 mg, 1.34 mmol) in one portion, followed by CBr₄ (332.46 mg, 1.00 mmol) in portions. The reaction mixture was stirred at 18° C. for 1 hour and was poured into water (10 mL) and extracted with dichloromethane (10 mL*3). The combined organic layers were concentrated under reduced pressure to give a residue. The crude was purified by prep-TLC to give 36f. MS mass calculated for [M+H]⁺(C₁₄H₁₁BrF₃NO₂) requires m/z, 362.0/364.0, LCMS found m/z, 361.9/363.9; ¹HNMR (CHLOROFORM-d, 400 MHz): δ=7.58-7.63 (m, 1H), 7.55 (dd, J=7.9, 1.8 Hz, 1H), 7.39-7.48 (m, 2H), 4.34 (s, 2H), 2.13 (tt, J=8.4, 5.1 Hz, 1H), 1.27-1.30 (m, 2H), 1.16-1.23 (m, 2H).

Tert-butyl 4-((5-cyclopropyl-3-(2-(trifluoromethoxy)phenyl)isoxazol-4-yl)methoxy)piperidine-1-carboxylate (36g). To a solution of 4-(bromomethyl)-5-cyclopropyl-3-(2-(trifluoromethoxy)phenyl)isoxazole (36f) (200 mg, 552.27 umol) and tert-butyl 4-hydroxypiperidine-1-carboxylate (26h) (144.50 mg, 717.95 umol) in THE (5 mL) was added 18-CROWN-6 (218.96 mg, 828.41 umol) and t-BuOK (1 M solution in THF, 828.41 uL) dropwise at 0° C. The mixture was stirred at 20° C. for 2 hours and was poured into water (10 ml) and extracted with ethyl acetate (10 mL*2). The combined organic layer was washed with brine (10 mL), dried over anhydrous Na₂SO₄, filtered and the filtrate was concentrated to give a residue which was purified by prep-TLC to give 36g. MS mass calculated for [M+H]⁺(C₂₄H₂₉F₃N₂O₅) requires m/z, 483.2 LCMS found m/z, 483.2; ¹H NMR (CHLOROFORM-d, 400 MHz): δ=7.40-7.55 (m, 2H), 7.31 (t, J=7.1 Hz, 2H), 4.29 (s, 2H), 3.46-3.58 (m, 2H), 3.31 (tt, J=7.9, 3.8 Hz, 1H), 2.86-2.99 (m, 2H), 2.05 (tt, J=8.5, 5.1 Hz, 1H), 1.58 (br d, J=2.7 Hz, 2H), 1.37 (s, 9H), 1.25-1.34 (m, 2H), 1.13-1.18 (m, 2H), 0.98-1.06 (m, 2H).

5-cyclopropyl-4-((piperidin-4-yloxy)methyl)-3-(2-(trifluoromethoxy)phenyl)isoxazole (36h). To a solution of tert-butyl 4-((5-cyclopropyl-3-(2-(trifluoromethoxy)phenyl)isoxazol-4-yl)methoxy)piperidine-1-carboxylate (36g) (200 mg, 414.52 umol) in ethyl acetate (2 mL) was added HCl/ethyl acetate (2 mL, 4M) at 20° C. for 2 hours. The reaction mixture was concentrated to give 36h. MS mass calculated for [M+H]⁺(C₁₉H₂₁F₃N₂O₃) requires m/z, 383.2 LCMS found m/z, 383.2.

4-(4-((5-cyclopropyl-3-(2-(trifluoromethoxy)phenyl)isoxazol-4-yl)methoxy)piperidin-1-yl)benzonitrile (36i). To a solution of 5-cyclopropyl-4-((piperidin-4-yloxy)methyl)-3-(2-(trifluoromethoxy)phenyl)isoxazole hydrochloride (36h) (100 mg, 238.76 umol) and 4-fluorobenzonitrile (2a) (144.58 mg, 1.19 mmol) in DMSO (2 mL) was added K₂CO₃ (98.99 mg, 716.27 umol) at 20° C. The mixture was stirred at 80° C. for 2 hours and was diluted with ethyl acetate (20 mL) and washed with brine (10 mL*2, 5 mL*2). The organic layer was dried over anhydrous Na₂SO₄ and concentrated. The residue was purified by prep-TLC to give 36i. MS mass calculated for [M+H]⁺(C₂₆H₂₄F₃N₃O₃) requires m/z, 484.2 LCMS found m/z, 484.2; ¹H NMR (CHLOROFORM-d, 400 MHz): δ=7.43-7.53 (m, 4H), 7.37 (d, J=7.9 Hz, 2H), 6.80 (d, J=8.8 Hz, 2H), 4.40 (s, 2H), 3.40-3.54 (m, 3H), 2.99-3.09 (m, 2H), 2.09-2.18 (m, 1H), 1.73-1.85 (m, 2H), 1.53-1.61 (m, 2H), 1.20-1.28 (m, 2H), 1.06-1.13 (m, 2H).

(Z)-4-(4-((5-cyclopropyl-3-(2-(trifluoromethoxy)phenyl)isoxazol-4-yl)methoxy)piperidin-1-yl)-N′-hydroxybenzimidamide (36j). To a solution of 4-(4-((5-cyclopropyl-3-(2-(trifluoromethoxy)phenyl)isoxazol-4-yl)methoxy)piperidin-1-yl)benzonitrile (36i) (100 mg, 206.83 umol) in ethanol (5 mL) was added hydroxylamine (0.5 mL, 50% in water) at 18° C. and the mixture was stirred at 80° C. for 2 hours. The reaction mixture was concentrated and the residue was then diluted with ethyl acetate (15 mL) and washed with brine (5 mL*2). The organic layer was dried over anhydrous Na₂SO₄, filtered and the filtrate was concentrated. The residue was purified by prep-TLC to give 36j. MS mass calculated for [M+H]⁺(C₂₆H₂₇F₃N₄O₄) requires m/z, 517.2 LCMS found m/z, 517.2.

3-(4-(4-((5-cyclopropyl-3-(2-(trifluoromethoxy)phenyl)isoxazol-4-yl)methoxy)piperidin-1-yl)phenyl)-1,2,4-oxadiazol-5(4H)-one (Compound 36). To a solution of (Z)-4-(4-((5-cyclopropyl-3-(2-(trifluoromethoxy)phenyl)isoxazol-4-yl)methoxy)piperidin-1-yl)-N′-hydroxybenzimidamide (36j) (50 mg, 96.80 umol) and diethyl carbonate (686.13 mg, 5.81 mmol, 703.72 uL) in ethanol (2 mL) was added CH₃ONa (139.45 mg, 774.43 umol, 25.81 uL, 30% in MeOH) at 18° C. The mixture was stirred at 80° C. for 2 hours and was concentrated to remove the solvents. The residue was diluted with ethyl acetate (20 mL) and washed with brine (10 mL*2), dried over anhydrous Na₂SO₄. Filtered and concentrated to give a residue, which was purified by prep-TLC to give Compound 36. MS mass calculated for [M+H]⁺(C₂₇H₂₅F₃N₄O₅) requires m/z, 543.2 LCMS found m/z, 543.2; ¹H NMR (CHLOROFORM-d, 400 MHz): δ=7.53 (d, J=8.9 Hz, 2H), 7.39-7.51 (m, 2H), 7.26-7.34 (m, 2H), 6.82 (d, J=8.9 Hz, 2H), 4.33 (s, 2H), 3.33-3.46 (m, 3H), 2.92-3.04 (m, 2H), 2.01-2.14 (m, 1H), 1.68-1.82 (m, 2H), 1.50 (dtd, J=12.5, 8.3, 3.7 Hz, 2H), 1.12-1.22 (m, 2H), 0.95-1.08 (m, 2H).

### Example 37

### 5-(6-(4-((5-cyclopropyl-3-(2,6-dichlorophenyl)isoxazol-4-yl)methoxy)piperidin-1-yl)pyridin-3-yl)-1,3,4-oxadiazol-2(3H)-one

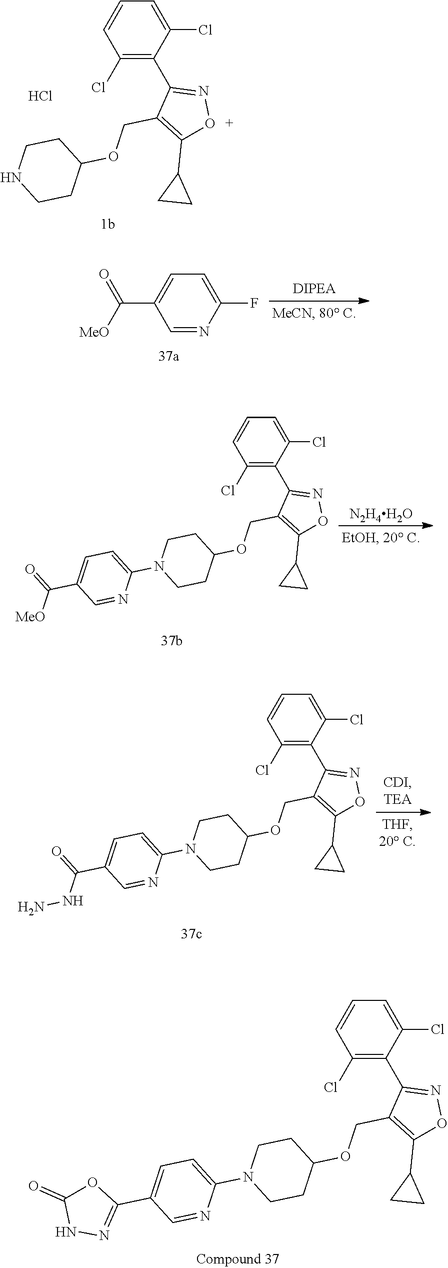

Methyl 6-(4-((5-cyclopropyl-3-(2,6-dichlorophenyl)isoxazol-4-yl)methoxy)piperidin-1-yl)nicotinate (37b). To a solution of 5-cyclopropyl-3-(2,6-dichlorophenyl)-4-((piperidin-4-yloxy)methyl)isoxazole hydrochloride (1b) (200 mg, 495.38 umol) and methyl 6-fluoronicotinate (37a) (153.69 mg, 990.76 umol) in CH₃CN (10 mL) was added DIPEA (320.12 mg, 2.48 mmol, 431.42 uL) at 20° C. The reaction was stirred at 80° C. for 16 hours and was concentrated under reduced pressure to remove solvent. The residue was diluted with water (5 mL) and ethyl acetate (5 mL) and the phases were separated. The aqueous phase was extracted with ethyl acetate (5 mL*4). The combined organic phase was washed with brine (20 mL*2), dried with anhydrous Na₂SO₄, filtered and the filtrate was concentrated under reduced pressure. The residue was purified by prep-TLC (SiO₂, petroleum ether:ethyl acetate=2:1) to give 37b. MS mass calculated for [M+H]⁺(C₂₅H₂₅Cl₂N₃O₄) requires m/z, 502.1/504.1, LCMS found m/z, 502.1/504.1; ¹H NMR (400 MHz, CHLOROFORM-d) δ=8.77 (d, J=2.1 Hz, 1H), 7.99 (dd, J=2.3, 9.0 Hz, 1H), 7.44-7.37 (m, 2H), 7.33-7.28 (m, 1H), 6.55 (d, J=9.0 Hz, 1H), 4.36 (s, 2H), 3.87 (s, 3H), 3.80-3.72 (m, 2H), 3.55-3.51 (m, 1H), 3.38-3.30 (m, 2H), 2.20-2.12 (m, 1H), 1.78-1.69 (m, 2H), 1.54-1.43 (m, 2H), 1.31-1.25 (m, 2H), 1.16-1.11 (m, 2H).

6-(4-((5-cyclopropyl-3-(2,6-dichlorophenyl)isoxazol-4-yl)methoxy)piperidin-1-yl)nicotinohydrazide (37c). To a solution of methyl 6-(4-((5-cyclopropyl-3-(2,6-dichlorophenyl)isoxazol-4-yl)methoxy)piperidin-1-yl)nicotinate (37b) (100 mg, 199.05 umol) in ethanol (4 mL) was added hydrazine hydrate (4.12 g, 82.30 mmol, 4 mL) at 20° C. The reaction was stirred at 20° C. for 6 hours and the mixture was concentrated under reduced pressure to remove solvent to give 37c. MS mass calculated for [M+H]⁺(C₂₄H₂₅Cl₂N₅O₃) requires m/z, 502.1/504.1, LCMS found m/z, 502.1/504.2.

5-(6-(4-((5-cyclopropyl-3-(2,6-dichlorophenyl)isoxazol-4-yl)methoxy)piperidin-1-yl)pyridin-3-yl)-1,3,4-oxadiazol-2(3H)-one (Compound 37). To a mixture of 6-(4-((5-cyclopropyl-3-(2,6-dichlorophenyl)isoxazol-4-yl)methoxy)piperidin-1-yl)nicotinohydrazide (37c) (100 mg, 199.05 umol) in THE (10 mL) was added CDI (64.55 mg, 398.10 umol), TEA (60.42 mg, 597.14 umol, 83.12 uL) at 20° C. The reaction mixture was stirred at 20° C. for 16 hours and was concentrated under reduced pressure to remove solvent. The residue was diluted with water (5 mL) and ethyl acetate (5 mL) and then the phases were separated. The aqueous phase was extracted with ethyl acetate (5 mL*4). The combined organic phase was washed with brine (20 mL*2), dried with anhydrous Na₂SO₄, filtered and concentrated under reduced pressure and purified by prep-HPLC (neutral condition: column: Waters Xbridge BEH C18 100*25 mm*5 um; mobile phase: [water(10 mM NH₄HCO₃)-ACN]; B %: 30%-60%, 10 min) to give Compound 37. MS mass calculated for [M+H]⁺(C₂₅H₂₃Cl₂N₅O₄) requires m/z, 528.1/530.1 LCMS found m/z 528.1/530.1; ¹H NMR (400 MHz, CHLOROFORM-d) δ=8.69-8.55 (m, 2H), 7.81 (dd, J=2.4, 9.0 Hz, 1H), 7.43-7.39 (m, 2H), 7.35-7.30 (m, 1H), 6.63 (d, J=9.0 Hz, 1H), 4.36 (s, 1H), 4.38-4.35 (m, 1H), 3.79-3.72 (m, 2H), 3.53 (tt, J=3.6, 7.3 Hz, 1H), 3.40-3.32 (m, 2H), 2.16 (tt, J=5.1, 8.4 Hz, 1H), 1.79-1.70 (m, 2H), 1.55-1.45 (m, 2H), 1.31-1.26 (m, 2H), 1.17-1.11 (m, 2H).

### Example 38

### 3-(5-(4-((5-cyclopropyl-3-(2,6-dichlorophenyl)isoxazol-4-yl)methoxy)piperidin-1-yl)thiophen-3-yl)-1,2,4-oxadiazol-5(4H)-one

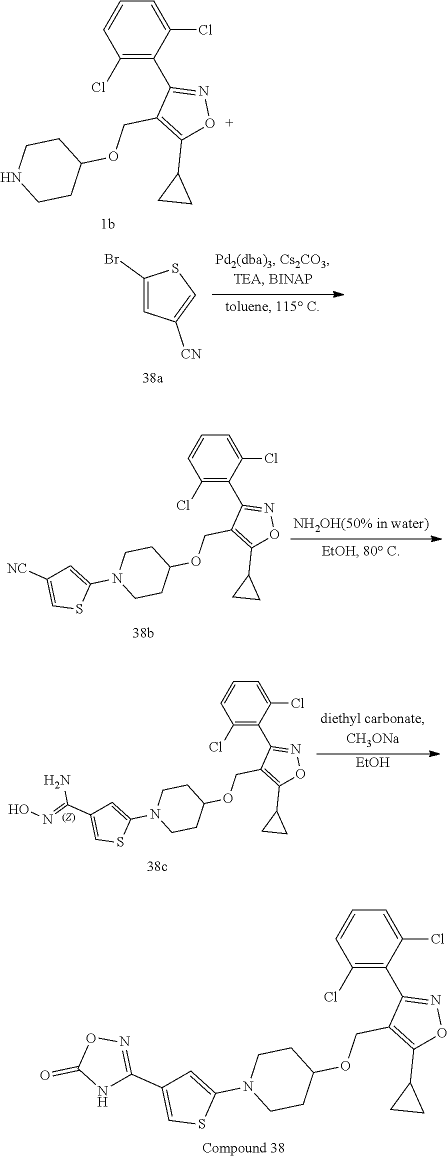

5-(4-((5-cyclopropyl-3-(2,6-dichlorophenyl)isoxazol-4-yl)methoxy)piperidin-1-yl)thiophene-3-carbonitrile (38b). To a mixture of 5-cyclopropyl-3-(2,6-dichlorophenyl)-4-((piperidin-4-yloxy)methyl)isoxazole hydrochloride (1b) (386.46 mg, 957.22 umol, HCl) and 5-bromothiophene-3-carbonitrile (38a) (120 mg, 638.15 umol) in toluene (20 mL) was added TEA (129.15 mg, 1.28 mmol, 177.64 uL), Pd₂(dba)₃ (29.22 mg, 31.91 umol), Cs₂CO₃ (1.25 g, 3.83 mmol) and [1-(2-diphenylphosphanyl-1-naphthyl)-2-naphthyl]-diphenyl-phosphane (476.83 mg, 765.77 umol) at 20° C. under N₂. The mixture was stirred at 115° C. for 12 hours and was poured into water (10 mL). The mixture was extracted with ethyl acetate (20 mL). The organic phase was washed with brine (10 mL*2), dried with anhydrous Na₂SO₄, filtered and concentrated under reduced pressure. The residue was purified by prep-TLC to give 38b. MS mass calculated for [M+H]⁺(C₂₃H₂₁Cl₂N₃O₂S) requires m/z, 474.1/476.1, LCMS found m/z, 474.0/476.0; ¹H NMR (400 MHz, CHLOROFORM-d) δ=7.42-7.35 (m, 2H), 7.33-7.28 (m, 1H), 7.15 (d, J=1.5 Hz, 1H), 6.10 (d, J=1.5 Hz, 1H), 4.34 (s, 2H), 3.45 (tt, J=3.3, 7.0 Hz, 1H), 3.10 (ddd, J=3.8, 7.9, 11.9 Hz, 2H), 2.91 (ddd, J=3.9, 7.6, 11.8 Hz, 2H), 2.14 (tt, J=5.1, 8.5 Hz, 1H), 1.83-1.73 (m, 2H), 1.67-1.58 (m, 2H), 1.31-1.25 (m, 2H), 1.16-1.09 (m, 2H).

(Z)-5-(4-((5-cyclopropyl-3-(2,6-dichlorophenyl)isoxazol-4-yl)methoxy)piperidin-1-yl)-N′-hydroxythiophene-3-carboximidamide (38c). To a mixture of 5-(4-((5-cyclopropyl-3-(2,6-dichlorophenyl)isoxazol-4-yl)methoxy)piperidin-1-yl)thiophene-3-carbonitrile (38b) (100 mg, 210.79 umol) in ethanol (3 mL) was added hydroxylamine (1 mL, 50% in water) in one portion at 25° C. under N₂. The mixture was stirred at 80° C. for 12 hours and was concentrated under reduced pressure. The residue was purified by prep-TLC to give 38c. MS mass calculated for [M+H]⁺(C₂₃H₂₄Cl₂N₄O₃S) requires m/z, 507.1/509.1, LCMS found m/z, 507.2/509.2.

3-(5-(4-((5-cyclopropyl-3-(2,6-dichlorophenyl)isoxazol-4-yl)methoxy)piperidin-1-yl)thiophen-3-yl)-1,2,4-oxadiazol-5(4H)-one (Compound 38). To a mixture of diethyl carbonate (975.00 mg, 8.25 mmol, 1 mL) and (Z)-5-(4-((5-cyclopropyl-3-(2,6-dichlorophenyl)isoxazol-4-yl)methoxy)piperidin-1-yl)-N′-hydroxythiophene-3-carboximidamide (38c) (80.00 mg, 157.59 umol) in ethanol (2 mL) was added CH₃ONa (283.91 mg, 1.58 mmol, 30% in MeOH) in one portion at 20° C. under N₂. The mixture was stirred at 80° C. for 12 hours and was poured into water (10 mL). The mixture was extracted with ethyl acetate (20 mL). The organic phase was washed with brine (20 mL), dried with anhydrous Na₂SO₄, filtered and concentrated under reduced pressure. The residue was purified by prep-HPLC (TFA condition) to give Compound 38. MS mass calculated for [M+H]⁺(C₂₄H₂₂Cl₂N₄O₄S) requires m/z, 533.1/535.1, LCMS found m/z, 533.1/535.1; ¹H NMR (400 MHz, CHLOROFORM-d) δ=11.01-10.48 (m, 1H), 7.47-7.38 (m, 2H), 7.37-7.30 (m, 1H), 7.14 (br s, 1H), 6.38 (br s, 1H), 4.37 (s, 2H), 3.49 (br s, 1H), 3.17 (br s, 2H), 2.98 (br s, 2H), 2.22-2.10 (m, 1H), 1.82 (br s, 2H), 1.68 (br s, 2H), 1.30 (br d, J=4.9 Hz, 2H), 1.16 (br d, J=7.5 Hz, 2H).

### Example 39

### 3-(4-(4-((5-cyclopropyl-3-(2,6-dichlorophenyl)isoxazol-4-yl)methoxy)piperidin-1-yl)-3-fluorophenyl)-1,2,4-oxadiazol-5(4H)-one

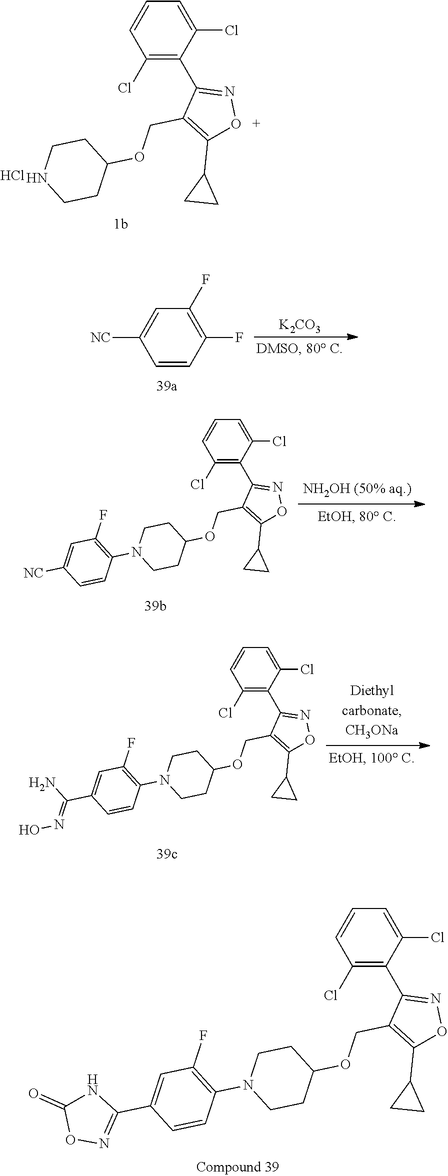

4-(4-((5-cyclopropyl-3-(2,6-dichlorophenyl)isoxazol-4-yl)methoxy)piperidin-1-yl)-3-fluorobenzonitrile (39b). To a solution of 5-cyclopropyl-3-(2,6-dichlorophenyl)-4-((piperidin-4-yloxy)methyl)isoxazole hydrochloride (1b) (100 mg, 247.69 umol) in DMSO (3 mL) was added K₂CO₃ (171.16 mg, 1.24 mmol) and 3,4-difluorobenzonitrile (39a) (103.36 mg, 743.07 umol) at 20° C. The mixture was heated to 80° C. for 16 hours and was poured into H₂O (10 mL). The mixture was extracted with ethyl acetate (20 mL*2). The combined organic layer was washed with brine (10 mL), dried over anhydrous Na₂SO₄, filtered and concentrated under reduced pressure to give a residue. The residue was purified by prep-TLC (Petroleum ether:ethyl acetate=3:1) to give 39b. MS mass calculated for [M+H]⁺(C₂₅H₂₂Cl₂FN₃O₂) requires m/z, 486.1/488.1, LCMS found m/z, 486.1/488.1; ¹H NMR (400 MHz, CHLOROFORM-d) δ=7.45-7.38 (m, 2H), 7.37-7.29 (m, 2H), 7.23 (s, 1H), 6.85 (t, J=8.6 Hz, 1H), 4.35 (s, 2H), 3.46 (td, J=3.8, 7.2 Hz, 1H), 3.26-3.16 (m, 2H), 2.92 (ddd, J=3.3, 8.2, 11.9 Hz, 2H), 2.20-2.11 (m, 1H), 1.87-1.77 (m, 2H), 1.66-1.57 (m, 2H), 1.32-1.24 (m, 2H), 1.17-1.10 (m, 2H).

(Z)-4-(4-((5-cyclopropyl-3-(2,6-dichlorophenyl)isoxazol-4-yl)methoxy)piperidin-1-yl)-3-fluoro-N′-hydroxybenzimidamide (39c). To a solution of 4-(4-((5-cyclopropyl-3-(2,6-dichlorophenyl)isoxazol-4-yl)methoxy)piperidin-1-yl)-3-fluorobenzonitrile (39b) (110 mg, 226.17 umol) in ethanol (5 mL) was added hydroxylamine (1 mL, 50% in water) at 20° C. and the mixture was heated to 80° C. for 2 hours. The reaction mixture was poured into H₂O (10 mL) and extracted with ethyl acetate (20 mL*2). The combined organic layer was washed with brine (10 mL), dried over anhydrous Na₂SO₄, filtered and concentrated under reduced pressure to give a residue. The residue was purified by prep-TLC (SiO₂, dichloromethane:Methanol=10:1) to give 39c. MS mass calculated for [M+H]⁺(C₂₅H₂₅Cl₂FN₄O₃) requires m/z, 519.1/521.1, LCMS found m/z, 519.1/521.2; ¹H NMR (400 MHz, CHLOROFORM-d) δ=7.44-7.37 (m, 2H), 7.35-7.30 (m, 2H), 7.29 (br d, J=2.5 Hz, 1H), 6.87 (t, J=8.5 Hz, 1H), 4.80 (br s, 2H), 4.35 (s, 2H), 3.42 (tt, J=3.7, 7.6 Hz, 1H), 3.18-3.08 (m, 2H), 2.80 (ddd, J=3.2, 8.5, 11.7 Hz, 2H), 2.21-2.13 (m, 1H), 1.88-1.78 (m, 2H), 1.68-1.57 (m, 2H), 1.31-1.25 (m, 2H), 1.17-1.10 (m, 2H).

3-(4-(4-((5-cyclopropyl-3-(2,6-dichlorophenyl)isoxazol-4-yl)methoxy)piperidin-1-yl)-3-fluorophenyl)-1,2,4-oxadiazol-5(4H)-one (Compound 39). To a solution of (Z)-4-(4-((5-cyclopropyl-3-(2,6-dichlorophenyl)isoxazol-4-yl)methoxy)piperidin-1-yl)-3-fluoro-N′-hydroxybenzimidamide (39c) (50 mg, 96.27 umol) in ethanol (2.5 mL) was added diethyl carbonate (487.50 mg, 4.13 mmol, 0.5 mL) and CH₃ONa (138.68 mg, 770.13 umol, 30% in MeOH) at 20° C. The mixture was heated to 100° C. for 1 hour and was poured into H₂O (10 mL). The mixture was extracted with ethyl acetate (20 mL*2). The combined organic layer was washed with brine (10 mL), dried over anhydrous Na₂SO₄, filtered and concentrated under reduced pressure to give a residue. The residue was purified by prep-TLC (SiO₂, dichloromethane:Methanol=10:1) to give Compound 39. MS mass calculated for [M+H]⁺(C₂₆H₂₃Cl₂FN₄O₄) requires m/z, 545.1/547.1, LCMS found m/z, 545.1/547.2; ¹H NMR (400 MHz, CHLOROFORM-d) δ=7.42-7.38 (m, 2H), 7.32-7.32 (m, 1H), 7.38-7.32 (m, 1H), 7.31-7.27 (m, 1H), 6.85 (br s, 1H), 4.35 (s, 2H), 3.44 (br s, 1H), 3.16 (br s, 2H), 2.86 (br s, 2H), 2.21-2.11 (m, 1H), 1.80 (br s, 2H), 1.60 (br d, J=8.2 Hz, 2H), 1.31-1.24 (m, 2H), 1.17-1.09 (m, 2H).

### Example 40

### 5-(5-(4-((5-cyclopropyl-3-(2,6-dichlorophenyl)isoxazol-4-yl)methoxy)piperidin-1-yl)pyridin-2-yl)-1,3,4-oxadiazol-2(3H)-one

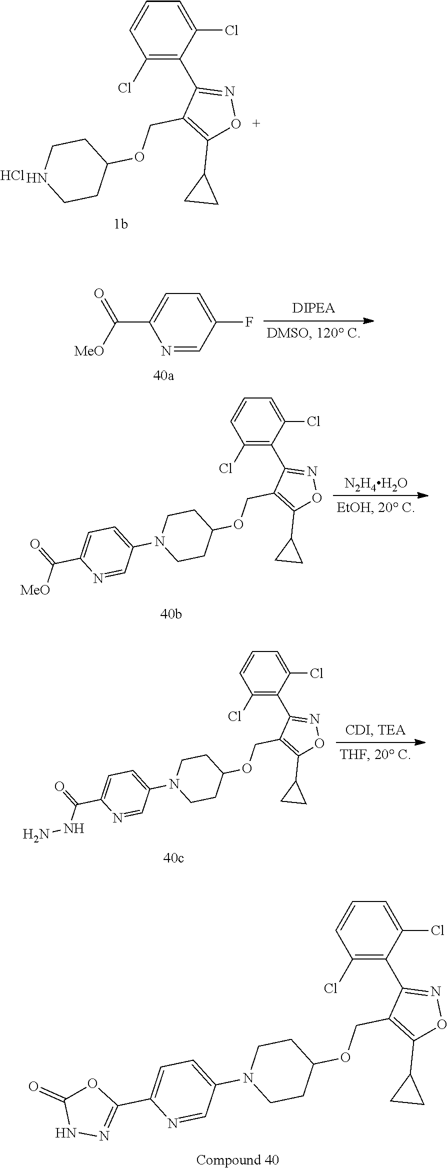

Methyl 5-(4-((5-cyclopropyl-3-(2,6-dichlorophenyl)isoxazol-4-yl)methoxy)piperidin-1-yl)picolinate (40b). To a solution of 5-cyclopropyl-3-(2,6-dichlorophenyl)-4-((piperidin-4-yloxy)methyl)isoxazole hydrochloride (1b) (250 mg, 619.23 umol) and methyl 5-fluoropicolinate (40a)(192.12 mg, 1.24 mmol) in DMSO (10 mL) was added DIPEA (320.12 mg, 2.48 mmol, 431.42 uL) at 20° C. The reaction was stirred at 120° C. for 16 hours and was concentrated under reduced pressure to remove solvent. Water (5 mL) and ethyl acetate (5 mL) were added into the residue and the phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, ethyl acetate) to give 40b. MS mass calculated for [M+H]⁺(C₂₅H₂₅Cl₂N₃O₄) requires m/z, 502.1/504.1, LCMS found m/z 502.1/504.1; ¹H NMR (400 MHz, CHLOROFORM-d) δ=8.28 (d, J=2.8 Hz, 1H), 7.98 (d, J=8.8 Hz, 1H), 7.41-7.35 (m, 2H), 7.31-7.28 (m, 1H), 7.09 (dd, J=2.9, 8.8 Hz, 1H), 4.35 (s, 2H), 3.97 (s, 3H), 3.52 (td, J=3.5, 7.0 Hz, 1H), 3.37-3.29 (m, 2H), 3.17-3.09 (m, 2H), 2.18-2.10 (m, 1H), 1.79 (dt, J=3.9, 8.6 Hz, 2H), 1.65-1.57 (m, 2H), 1.27 (t, J=3.0 Hz, 2H), 1.17-1.11 (m, 2H).

5-(4-((5-cyclopropyl-3-(2,6-dichlorophenyl)isoxazol-4-yl)methoxy)piperidin-1-yl)picolinohydrazide (40c). To a solution of methyl 5-(4-((5-cyclopropyl-3-(2,6-dichlorophenyl)isoxazol-4-yl)methoxy)piperidin-1-yl)picolinate (40b) (100 mg, 199.05 umol) in ethanol (6 mL) was added hydrazine hydrate (3.09 g, 61.73 mmol, 3 mL) at 20° C. and the reaction was stirred at 20° C. for 3 hours. The reaction mixture was concentrated under reduced pressure to remove solvent to give 40c. MS mass calculated for [M+H]⁺(C₂₄H₂₅Cl₂N₅O₃) requires m/z, 502.1/504.1, LCMS found m/z 502.1/504.1.

5-(5-(4-((5-cyclopropyl-3-(2,6-dichlorophenyl)isoxazol-4-yl)methoxy)piperidin-1-yl)pyridin-2-yl)-1,3,4-oxadiazol-2(3H)-one (Compound 40). A mixture of 5-(4-((5-cyclopropyl-3-(2,6-dichlorophenyl)isoxazol-4-yl)methoxy)piperidin-1-yl)picolinohydrazide (40c) (100 mg, 199.05 umol) in THE (2 mL) was added CDI (64.55 mg, 398.10 umol) and TEA (60.42 mg, 597.14 umol, 83.12 uL) at 20° C. and stirred for 16 hours. The reaction mixture was concentrated under reduced pressure to remove solvent. The residue was diluted with water (5 mL) and ethyl acetate (5 mL) and the phases were separated. The aqueous phase was extracted with ethyl acetate (5 mL*4). The combined organic phase was washed with brine (20 mL*2), dried with anhydrous Na₂SO₄, filtered and concentrated under reduced pressure and purified by prep-HPLC (neutral condition: column: Waters Xbridge BEH C18 100*25 mm*5 um; mobile phase: [water(10 mM NH₄HCO₃)-ACN]; B %: 25%-55%, 10 min) to give Compound 40. MS mass calculated for [M+H]⁺(C₂₅H₂₃Cl₂N₅O₄) requires m/z, 528.1/530.1, LCMS found m/z 528.1/530.2; ¹H NMR (400 MHz, CHLOROFORM-d) δ=8.35 (d, J=2.8 Hz, 1H), 7.68 (d, J=8.9 Hz, 1H), 7.42-7.37 (m, 2H), 7.30 (dd, J=7.1, 8.9 Hz, 1H), 7.13 (dd, J=2.9, 8.9 Hz, 1H), 4.36 (s, 2H), 3.52 (td, J=3.5, 6.9 Hz, 1H), 3.34 (ddd, J=3.8, 8.2, 12.3 Hz, 2H), 3.16-3.09 (m, 2H), 2.19-2.11 (m, 1H), 1.79 (dt, J=4.0, 8.4 Hz, 2H), 1.63-1.59 (m, 2H), 1.31-1.26 (m, 2H), 1.17-1.11 (m, 2H).

### Example 41

### 6-(4-(4-((5-cyclopropyl-3-(2,6-dichlorophenyl)isoxazol-4-yl)methoxy)piperidin-1-yl)-2-fluorophenyl)-1,2,4-triazine-3,5(2H,4H)-dione

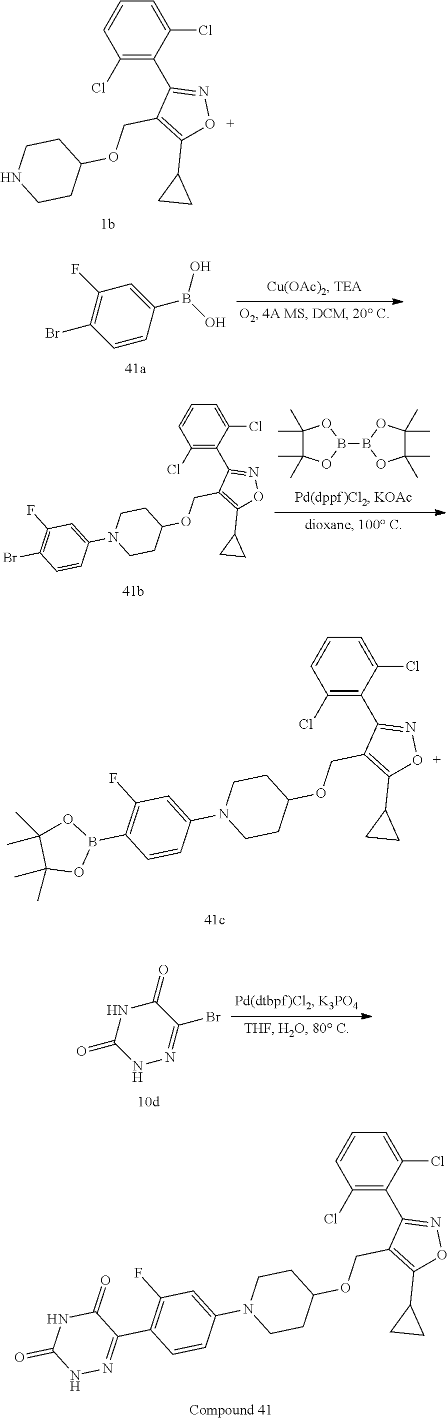

4-(((1-(4-bromo-3-fluorophenyl)piperidin-4-yl)oxy)methyl)-5-cyclopropyl-3-(2,6-dichlorophenyl)isoxazole (41b). To a solution of 5-cyclopropyl-3-(2,6-dichlorophenyl)-4-((piperidin-4-yloxy)methyl)isoxazole hydrochloride (1b) (120 mg, 326.74 umol) and (4-bromo-3-fluorophenyl)boronic acid (41a) (121.54 mg, 555.45 umol) in dichloromethane (8 mL) was added was added Cu(OAc)₂ (71.22 mg, 392.08 umol), TEA (66.12 mg, 653.47 umol, 90.95 uL) and Molecular sieve 4 A (50 mg) at 20° C. and the mixture was stirred at 20° C. for 16 hours under O₂ atmosphere. The reaction mixture was filtered and the filtrate was washed with H₂O (10 mL), brine (10 mL), dried over anhydrous Na₂SO₄. The mixture was filtered and the filtrate was concentrated under reduced pressure to give a residue. The residue was purified by prep-TLC (SiO₂, petroleum ether:ethyl acetate=3:1) to give 41b. MS mass calculated for [M+H]⁺(C₂₄H₂₂BrCl₂FN₂O₂) requires m/z, 541.0/539.0, LCMS found m/z, 541.0/539.0; ¹H NMR (400 MHz, CHLOROFORM-d) δ=7.42-7.36 (m, 2H), 7.34-7.28 (m, 2H), 6.59 (dd, J=2.7, 12.1 Hz, 1H), 6.52 (dd, J=2.6, 8.9 Hz, 1H), 4.34 (s, 2H), 3.43 (tt, J=3.7, 7.5 Hz, 1H), 3.26-3.16 (m, 2H), 2.93-2.87 (m, 2H), 2.20-2.11 (m, 1H), 1.82-1.72 (m, 2H), 1.61-1.50 (m, 2H), 1.31-1.24 (m, 2H), 1.17-1.09 (m, 2H).

5-cyclopropyl-3-(2,6-dichlorophenyl)-4-(((1-(3-fluoro-4-(4,4,5,5-tetramethyl-1,3,2-dioxaborolan-2-yl)phenyl)piperidin-4-yl)oxy)methyl)isoxazole (41c). To a solution of 4-(((1-(4-bromo-3-fluorophenyl)piperidin-4-yl)oxy)methyl)-5-cyclopropyl-3-(2,6-dichlorophenyl)isoxazole (41b) (50 mg, 92.55 umol) in 1,4-dioxane (5 mL) was added 4,4,5,5-tetramethyl-2-(4,4,5,5-tetramethyl-1,3,2-dioxaborolan-2-yl)-1,3,2-dioxaborolane (70.51 mg, 277.65 umol), Pd(dppf)Cl₂ (6.77 mg, 9.25 umol) and KOAc (18.17 mg, 185.10 umol) at 20° C. The mixture was heated to 100° C. and stirred for 16 hours then was poured into H₂O (10 mL) and extracted with ethyl acetate (20 mL*2). The combined organic layer was washed with brine (10 mL), dried over anhydrous Na₂SO₄, filtered and the filtrate was concentrated under reduced pressure to give a residue. The residue was purified by prep-TLC (SiO₂, petroleum ether:ethyl acetate=3:1) to give 41c. MS mass calculated for [M+H]⁺(C₃₀H₃₄BCl₂FN₂O₄) requires m/z, 587.2/589.2, LCMS found m/z, 587.2, 589.2.

6-(4-(4-((5-cyclopropyl-3-(2,6-dichlorophenyl)isoxazol-4-yl)methoxy)piperidin-1-yl)-2-fluorophenyl)-1,2,4-triazine-3,5(2H,4H)-dione (Compound 41). To a solution of 41c (30 mg, 51.08 umol) and 6-bromo-1,2,4-triazine-3,5(2H,4H)-dione (10d) (29.42 mg, 153.24 umol) in THE (2 mL) and H₂O (0.5 mL) was added K₃PO₄ (21.69 mg, 102.16 umol) and ditertbutyl(cyclopentyl)phosphane; dichloropalladium; iron (3.33 mg, 5.11 umol) at 20° C. The mixture was heated to 80° C. for 16 hours and was poured into H₂O (5 mL). The mixture was extracted with ethyl acetate (10 mL*3). The combined organic layer was washed with brine (5 mL), dried over anhydrous Na₂SO₄, filtered and concentrated under reduced pressure to give a residue. The residue was purified by prep-TLC (SiO₂, dichloromethane:methanol=10:1) to give Compound 41. MS mass calculated for [M+H]⁺(C₂₇H₂₄Cl₂FN₅O₄) requires m/z, 572.1/574.1, LCMS found m/z, 572.2/574.1; ¹H NMR (400 MHz, CHLOROFORM-d) δ=9.38 (br s, 1H), 8.58 (br s, 1H), 7.44-7.36 (m, 3H), 7.32 (br d, J=7.3 Hz, 1H), 6.65 (br d, J=8.9 Hz, 1H), 6.56 (br d, J=13.9 Hz, 1H), 4.35 (s, 2H), 3.47 (br s, 1H), 3.37-3.27 (m, 2H), 3.01 (br t, J=8.6 Hz, 2H), 2.20-2.11 (m, 1H), 1.77 (br d, J=9.1 Hz, 2H), 1.54 (br d, J=8.5 Hz, 2H), 1.26 (br d, J=3.6 Hz, 2H), 1.18-1.09 (m, 2H).

### Example 42

### 3-(3-(4-((5-cyclopropyl-3-(2,6-dichlorophenyl)isoxazol-4-yl)methoxy)piperidin-1-yl)isoxazol-5-yl)-1,2,4-oxadiazol-5(4H)-one

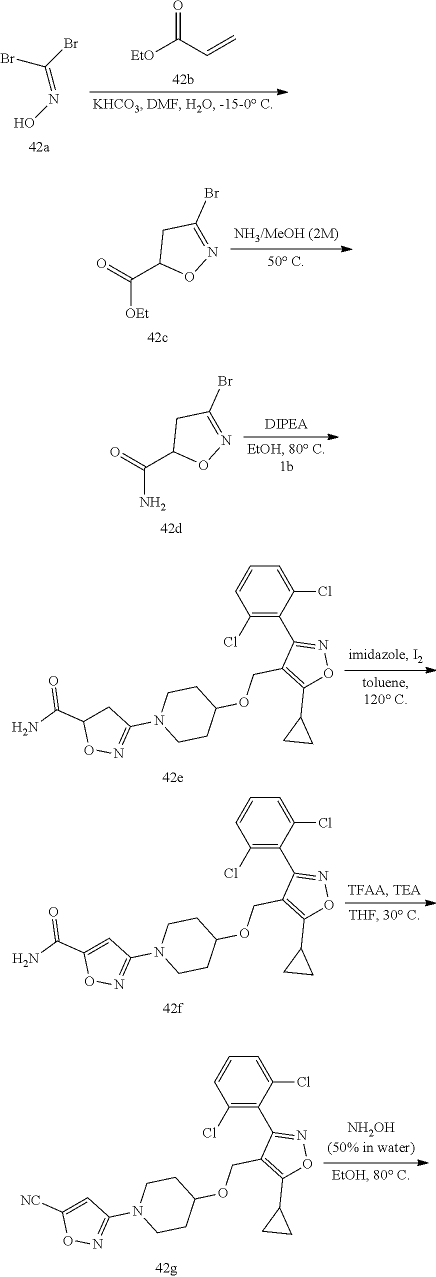

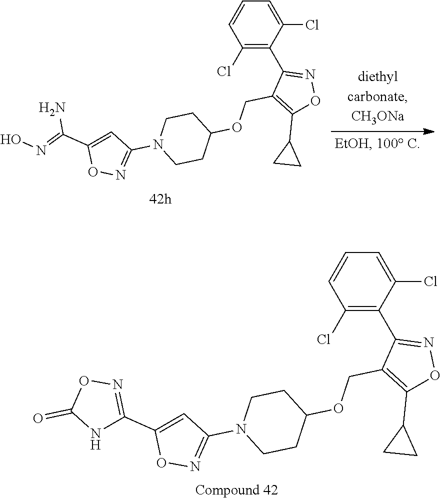 [SMILES: N/C(=N\O)c1cc(N2CCC(OCc3c(-c4c(Cl)cccc4Cl)noc3C3CC3)CC2)no1 ; O=c1[nH]c(-c2cc(N3CCC(OCc4c(-c5c(Cl)cccc5Cl)noc4C4CC4)CC3)no2)no1]

Ethyl 3-bromo-4,5-dihydroisoxazole-5-carboxylate (42c). To a solution of hydroxycarbonimidic dibromide (42a) (1 g, 4.93 mmol) in DMF (3 mL) at −15° C. was added ethyl acrylate (42b) (592.30 mg, 5.92 mmol, 643.11 uL) and KHCO₃ (987.17 mg, 9.86 mmol) in H₂O (4 mL) over 15 minutes (internal temperature rising to 0° C.). The mixture was stirred at 0° C. for 1 hour and water (5 mL) and MTBE (5 mL) were added into the reaction mixture. The phases were separated and the aqueous phase was extracted with MTBE (5 mL*4). The combined organic phase was washed with brine (10 mL*2), dried with anhydrous Na₂SO₄, filtered and the filtrate was concentrated under reduced pressure to give 42c. MS mass calculated for [M+H]⁺(C₆H₈BrNO₃) requires m/z, 222.0/224.0, LCMS found m/z 221.9/223.9; ¹H NMR (400 MHz, Acetone) δ=5.20 (br dd, J=7.0, 11.5 Hz, 1H), 4.22 (q, J=7.1 Hz, 2H), 3.77-3.63 (m, 1H), 3.61-3.48 (m, 1H), 1.27 (br t, J=7.1 Hz, 3H).

3-bromo-4,5-dihydroisoxazole-5-carboxamide (42d). A solution of ethyl 3-bromo-4,5-dihydroisoxazole-5-carboxylate (42c) (960 mg, 4.32 mmol) in NH₃/Methanol (15 mL, 2 M) was stirred at 50° C. for 2 hours. The reaction mixture was concentrated under reduced pressure to remove solvent to give 42c. ¹H NMR (400 MHz, Acetone) δ=7.35-6.58 (m, 2H), 5.08 (dd, J=6.4, 11.7 Hz, 1H), 3.71-3.59 (m, 1H), 3.53-3.42 (m, 1H).

3-(4-((5-cyclopropyl-3-(2,6-dichlorophenyl)isoxazol-4-yl)methoxy)piperidin-1-yl)-4,5-dihydroisoxazole-5-carboxamide (42e). To a mixture of 3-bromo-4,5-dihydroisoxazole-5-carboxamide (42d) (540 mg, 2.80 mmol) and 5-cyclopropyl-3-(2,6-dichlorophenyl)-4-((piperidin-4-yloxy)methyl)isoxazole hydrochloride (1b)(1.36 g, 3.36 mmol) in ethanol (15 mL) was added DIPEA (1.27 g, 9.79 mmol, 1.71 mL) at 20° C. The reaction was degassed and purged with N₂ 3 times and stirred at 80° C. for 16 hours. The reaction mixture was concentrated under reduced pressure to remove solvent. The residue was diluted with water (5 mL) and ethyl acetate (5 mL) and then the phases were separated. The aqueous phase was extracted with ethyl acetate (5 mL*4). The combined organic phase was washed with brine (20 mL*2), dried with anhydrous Na₂SO₄, filtered and the filtrate was concentrated under reduced pressure which was purified by column chromatography (SiO₂, dichloromethane:Methanol=10:1) to give 42e. MS mass calculated for [M+H]⁺(C₂₂H₂₄Cl₂N₄O₄) requires m/z, 479.1/481.1, LCMS found m/z 479.1/481.1; ¹H NMR (400 MHz, CHLOROFORM-d) δ=7.44-7.40 (m, 2H), 7.38-7.32 (m, 1H), 6.87 (br s, 1H), 5.52 (br s, 1H), 4.89 (dd, J=5.8, 9.1 Hz, 1H), 4.31 (s, 2H), 3.42 (tt, J=3.4, 7.2 Hz, 1H), 3.29-3.25 (m, 2H), 3.25-3.17 (m, 2H), 3.00-2.87 (m, 2H), 2.17-2.09 (m, 1H), 1.72-1.63 (m, 2H), 1.51-1.42 (m, 2H), 1.27 (dd, J=2.5, 5.0 Hz, 2H), 1.16-1.10 (m, 2H).

3-(4-((5-cyclopropyl-3-(2,6-dichlorophenyl)isoxazol-4-yl)methoxy)piperidin-1-yl)isoxazole-5-carboxamide (42f). To a solution of 3-(4-((5-cyclopropyl-3-(2,6-dichlorophenyl)isoxazol-4-yl)methoxy)piperidin-1-yl)-4,5-dihydroisoxazole-5-carboxamide (42e) (200 mg, 417.23 umol) and imidazole (85.21 mg, 1.25 mmol) in toluene (6 mL) was added iodine (158.84 mg, 625.84 umol, 126.07 uL) at 20° C. The mixture was stirred at 120° C. for 16 hours in a sealed tube. Sodium sulfite solution (5 mL) and ethyl acetate (5 mL) were added into the reaction mixture and the phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, ethyl acetate) to give 42f. MS mass calculated for [M+H]⁺(C₂₂H₂₂Cl₂N₄O₄) requires m/z, 477.1/479.1, LCMS found m/z 477.1/479.1; ¹H NMR (400 MHz, CHLOROFORM-d) δ=7.45-7.38 (m, 2H), 7.36-7.29 (m, 1H), 6.56 (s, 1H), 6.39 (br s, 1H), 5.70 (br s, 1H), 4.34 (s, 2H), 3.53-3.42 (m, 1H), 3.36-3.27 (m, 2H), 3.07-2.97 (m, 2H), 2.15 (tt, J=5.0, 8.5 Hz, 1H), 1.79-1.70 (m, 2H), 1.58-1.48 (m, 2H), 1.31-1.24 (m, 2H), 1.16-1.09 (m, 2H).

3-(4-((5-cyclopropyl-3-(2,6-dichlorophenyl)isoxazol-4-yl)methoxy)piperidin-1-yl)isoxazole-5-carbonitrile (42g). To a solution of 3-(4-((5-cyclopropyl-3-(2,6-dichlorophenyl)isoxazol-4-yl)methoxy)piperidin-1-yl)isoxazole-5-carboxamide (42f) (100 mg, 209.49 umol) in THF (5 mL) was added TFAA (132.00 mg, 628.48 umol, 87.42 uL), TEA (84.79 mg, 837.98 umol, 116.64 uL) at 20° C. and the mixture was stirred at 30° C. for 4 hours. The reaction mixture was concentrated under reduced pressure to remove solvent. Water (5 mL) and ethyl acetate (5 mL) were added into the residue and the phases were separated. The aqueous phase was extracted with ethyl acetate (5 mL*4). The combined organic phase was washed with brine (20 mL*2), dried with anhydrous Na₂SO₄, filtered and the filtrate was concentrated under reduced pressure. The residue was purified by prep-TLC (SiO₂, petroleum ether:ethyl acetate=1:1) to give 42g. MS mass calculated for [M+H]⁺(C₂₂H₂OCl₂N₄O₃) requires m/z, 459.1/461.1, LCMS found m/z 459.1/461.1; ¹H NMR (400 MHz, CHLOROFORM-d) δ=7.44-7.38 (m, 2H), 7.35-7.30 (m, 1H), 6.51 (s, 1H), 4.34 (s, 2H), 3.48 (tt, J=3.4, 7.1 Hz, 1H), 3.34-3.26 (m, 2H), 3.08-3.00 (m, 2H), 2.18-2.10 (m, 1H), 1.79-1.69 (m, 2H), 1.56-1.52 (m, 2H), 1.29-1.25 (m, 2H), 1.15-1.10 (m, 2H).

(Z)-3-(4-((5-cyclopropyl-3-(2,6-dichlorophenyl)isoxazol-4-yl)methoxy)piperidin-1-yl)-N′-hydroxyisoxazole-5-carboximidamide (42h). To a solution of 3-(4-((5-cyclopropyl-3-(2,6-dichlorophenyl)isoxazol-4-yl)methoxy)piperidin-1-yl)isoxazole-5-carbonitrile (42g) (60 mg, 130.63 umol) in ethanol (8 mL) was added hydroxylamine (3 mL, 50% in water) at 20° C. The reaction was degassed and purged with N₂ for 3 times and stirred at 80° C. for 4 hours under N₂ atmosphere. The reaction mixture was concentrated under reduced pressure to remove solvent. Water (5 mL) and ethyl acetate (5 mL) were added into the residue and the phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, ethyl acetate) to give 42h. ¹H NMR (400 MHz, CHLOROFORM-d) δ=7.44-7.38 (m, 2H), 7.35-7.29 (m, 1H), 6.90 (br s, 1H), 6.19 (s, 1H), 5.01 (br s, 2H), 4.33 (s, 2H), 3.44 (td, J=3.9, 7.4 Hz, 1H), 3.36-3.28 (m, 1H), 3.36-3.28 (m, 1H), 2.99 (ddd, J=3.6, 8.4, 12.5 Hz, 2H), 2.19-2.11 (m, 1H), 1.79-1.69 (m, 2H), 1.52 (dtd, J=3.9, 8.2, 12.5 Hz, 2H), 1.30-1.26 (m, 2H), 1.16-1.10 (m, 2H).

3-(3-(4-((5-cyclopropyl-3-(2,6-dichlorophenyl)isoxazol-4-yl)methoxy)piperidin-1-yl)isoxazol-5-yl)-1,2,4-oxadiazol-5(4H)-one (Compound 42). To a mixture of (Z)-3-(4-((5-cyclopropyl-3-(2,6-dichlorophenyl)isoxazol-4-yl)methoxy)piperidin-1-yl)-N′-hydroxyisoxazole-5-carboximidamide (42h) (55 mg, 111.71 umol) in ethanol (4 mL) was added diethyl carbonate (585.00 mg, 4.95 mmol, 0.6 mL) and CH₃ONa (120.69 mg, 670.25 umol, 1.5 mL, 30% in MeOH) at 20° C. in a sealed tube. The reaction mixture was stirred at 100° C. for 4 hours and was concentrated under reduced pressure to remove solvent. Water (5 mL) and ethyl acetate (5 mL) were added into the residue and the phases were separated. The aqueous phase was extracted with ethyl acetate (5 mL*4). The combined organic phase was washed with brine (20 mL*2), dried with anhydrous Na₂SO₄, filtered and concentrated under reduced pressure and purified by prep-HPLC (neutral condition) to give Compound 42. MS mass calculated for [M+H]⁺(C₂₃H₂₁Cl₂N₅O₅) requires m/z, 518.1/520.1, LCMS found m/z 518.1/520.1; ¹H NMR (400 MHz, CHLOROFORM-d) δ=7.39-7.34 (m, 2H), 7.30 (s, 1H), 6.38 (s, 1H), 4.30 (s, 2H), 3.37 (br s, 1H), 3.21 (br s, 2H), 2.91 (br s, 2H), 2.17-2.08 (m, 1H), 1.66 (br s, 2H), 1.44 (br d, J=6.7 Hz, 2H), 1.28-1.19 (m, 2H), 1.15-1.06 (m, 2H) MS mass calculated for [M+H]⁺(C₂₃H₂₁Cl₂N₅O₅) requires m/z, 518.1/520.1, LCMS found m/z 518.1/520.1; ¹H NMR (400 MHz, CHLOROFORM-d) δ=7.39-7.34 (m, 2H), 7.30 (s, 1H), 7.26 (s, 1H), 6.38 (s, 1H), 4.30 (s, 2H), 3.37 (br s, 1H), 3.21 (br s, 2H), 2.91 (br s, 2H), 2.17-2.08 (m, 1H), 1.66 (br s, 2H), 1.44 (br d, J=6.7 Hz, 2H), 1.28-1.19 (m, 2H), 1.15-1.06 (m, 2H).

### Example 43

### 6-(6-(4-((5-cyclopropyl-3-(2,6-dichlorophenyl)isoxazol-4-yl)methoxy)piperidin-1-yl)pyridin-3-yl)-1,2,4-triazine-3, 5(2H,4H)-dione

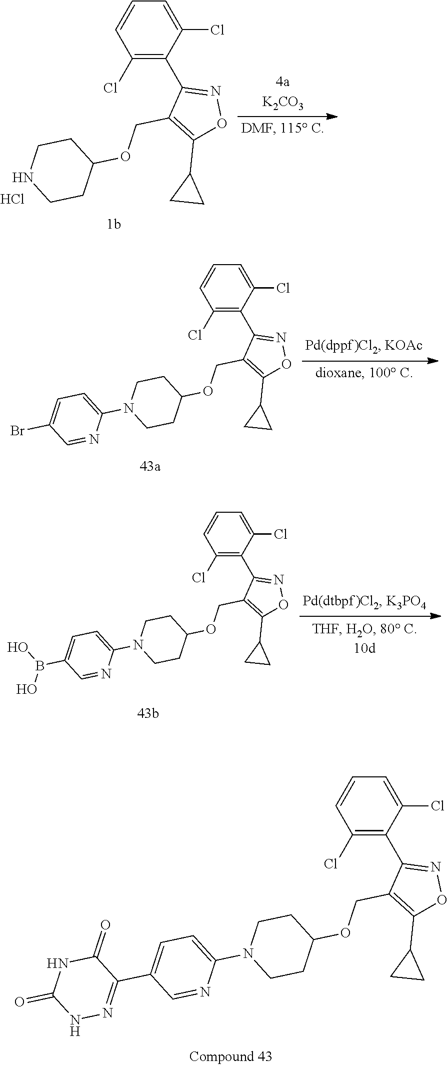 [SMILES: Cl ; Clc1cccc(Cl)c1-c1noc(C2CC2)c1COC1CCNCC1 ; Clc1cccc(Cl)c1-c1noc(C2CC2)c1COC1CCN(c2ccc(Br)cn2)CC1 ; OB(O)c1ccc(N2CCC(OCc3c(-c4c(Cl)cccc4Cl)noc3C3CC3)CC2)nc1 ; O=c1[nH]nc(-c2ccc(N3CCC(OCc4c(-c5c(Cl)cccc5Cl)noc4C4CC4)CC3)nc2)c(=O)[nH]1]

4-(((1-(5-bromopyridin-2-yl)piperidin-4-yl)oxy)methyl)-5-cyclopropyl-3-(2,6-dichlorophenyl)isoxazole (43a). To a solution of 5-cyclopropyl-3-(2,6-dichlorophenyl)-4-((piperidin-4-yloxy)methyl)isoxazole hydrochloride (1b) (200 mg, 495.38 umol) and 5-bromo-2-fluoropyridine (4a) (95.90 mg, 544.92 umol, 56.08 uL) in DMF (4 mL) was added K₂CO₃ (205.39 mg, 1.49 mmol). The mixture was heated to 115° C. and stirred for 18 hours under N₂. The reaction mixture was diluted with water (5 mL) and extracted with ethyl acetate (10 mL*3). The combined organic phase was washed with brine (5 mL*3), dried over anhydrous Na₂SO₄, filtered and the filtrated was concentrated under reduced pressure. The residue was purified by column chromatography (SiO₂, petroleum ether:ethyl acetate=10:1 to 5:1) to give 43a. MS mass calculated for [M+H]⁺(C₂₃H₂₂BrCl₂N₃O₂) requires m/z, 524.0/522.0, LCMS found m/z, 524.0/522.1; ¹H NMR (400 MHz, CHLOROFORM-d) δ=8.16 (d, J=2.4 Hz, 1H), 7.50 (dd, J=2.4, 9.3 Hz, 1H), 7.45-7.36 (m, 2H), 7.35-7.28 (m, 1H), 6.51 (d, J=9.3 Hz, 1H), 4.35 (s, 2H), 3.72-3.58 (m, 2H), 3.46 (tt, J=3.5, 7.7 Hz, 1H), 3.14 (ddd, J=3.7, 8.8, 13.0 Hz, 2H), 2.21-2.10 (m, 1H), 1.80-1.66 (m, 2H), 1.47 (dtd, J=3.9, 8.4, 12.6 Hz, 2H), 1.32-1.22 (m, 2H), 1.20-1.05 (m, 2H).

(6-(4-((5-cyclopropyl-3-(2,6-dichlorophenyl)isoxazol-4-yl)methoxy)piperidin-1-yl)pyridin-3-yl)boronic acid (43b). To a solution of 4-(((1-(5-bromopyridin-2-yl)piperidin-4-yl)oxy)methyl)-5-cyclopropyl-3-(2,6-dichlorophenyl)isoxazole (43a) (200 mg, 382.23 umol) and 4,4,5,5-tetramethyl-2-(4,4,5,5-tetramethyl-1,3,2-dioxaborolan-2-yl)-1,3,2-dioxaborolane (194.12 mg, 764.45 umol) in 1, 4-dioxane (4 mL) was added Pd(dppf)Cl₂ (55.94 mg, 76.45 umol) and KOAc (75.02 mg, 764.45 umol) under N₂. The resulting mixture was degassed and purged with N₂ 3 times and heated to 100° C. and stirred for 18 hours. The reaction mixture was cooled to 45° C. and diluted with ethyl acetate (5 mL). 3-Mercaptopropyl-functionalized silica gel (100 mg) was added to the mixture. The mixture was stirred for 1 hour and filtered through a Celite pad. The filter cake was rinsed with ethyl acetate (10 mL*2) and the combined filtrate was concentrated under reduced pressure. The residue was purified by column chromatography (SiO₂, dichloromethane:methanol=20:1 to 15:1) to give 43b. MS mass calculated for [M+H]⁺(C₂₃H₂₄BCl₂N₃O₄) requires m/z, 488.1/490.1, LCMS found m/z, 488.0/490.0; ¹H NMR (400 MHz, CHLOROFORM-d) δ=8.87 (br s, 1H), 8.07 (br s, 1H), 7.41 (br d, J=7.8 Hz, 3H), 6.64 (br d, J=8.3 Hz, 1H), 4.36 (br d, J=8.3 Hz, 2H), 3.79 (br s, 3H), 3.52 (br s, 1H), 3.33 (m, 1H), 2.16 (br s, 1H), 1.75 (m, 1H), 1.56 (m, 3H), 1.27 (br s, 2H), 1.15 (br s, 2H).

6-(6-(4-((5-cyclopropyl-3-(2,6-dichlorophenyl)isoxazol-4-yl)methoxy)piperidin-1-yl)pyridin-3-yl)-1,2,4-triazine-3,5(2H,4H)-dione (Compound 43). To a solution of (6-(4-((5-cyclopropyl-3-(2,6-dichlorophenyl)isoxazol-4-yl)methoxy)piperidin-1-yl)pyridine-3-yl)boronic acid (43 b) (150 mg, 263.01 umol) and 6-bromo-1,2,4-triazine-3,5(2H,4H)-dione (10d) (100.98 mg, 526.03 umol) in THE (4 mL) and H₂O (1 mL) was added K₃PO₄ (111.66 mg, 526.03 umol) and ditert-butyl(cyclopentyl)phosphane; dichloropalladium; iron (17.14 mg, 26.30 umol). The reaction mixture was degassed and purged with N₂ 3 times and heated to 80° C. and stirred for 18 hours. The reaction mixture was diluted with ethyl acetate (5 mL). 3-Mercaptopropyl-functionalized silica gel (100 mg) was added. The mixture was stirred for 2 hours at 45° C. and it was filtered through a Celite pad. The filtrate was concentrated under reduced pressure. The residue was purified by prep-TLC (SiO₂, dichloromethane:methanol=10:1) and repurified by Prep-HPLC (neutral condition, column: Phenomenex Gemini-NX C18 75*30 mm*3 um; mobile phase: [water (10 mM NH₄HCO₃)-ACN]; B %: 35%-55%, 8 min) to give Compound 43. MS mass calculated for [M+H]⁺(C₂₆H₂₄Cl₂N₆O₄) requires m/z, 555.1/557.1, LCMS found m/z, 555.2/557.1; ¹H NMR (400 MHz, CHLOROFORM-d) δ=9.51 (br s, 1H), 8.88 (d, J=2.0 Hz, 1H), 8.08 (dd, J=2.1, 9.2 Hz, 1H), 7.45-7.28 (m, 3H), 6.62 (d, J=8.8 Hz, 1H), 4.36 (s, 2H), 3.84-3.70 (m, 2H), 3.56-3.46 (m, 1H), 3.37-3.24 (m, 2H), 2.21-2.11 (m, 1H), 1.74 (br d, J=3.5 Hz, 2H), 1.54-1.44 (m, 2H), 1.31-1.23 (m, 2H), 1.18-1.06 (m, 2H).

### Example 44

### 3-(4-(4-((3-(2-chlorophenyl)-5-cyclopropylisoxazol-4-yl)methoxy)piperidin-1-yl)phenyl)-1,2,4-oxadiazol-5(4H)-one

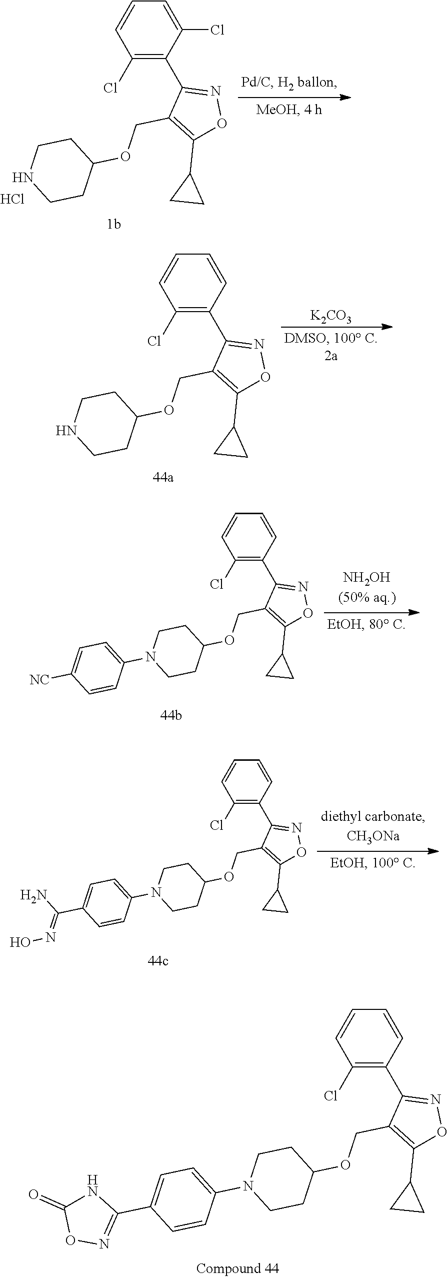

3-(2-chlorophenyl)-5-cyclopropyl-4-((piperidin-4-yloxy)methyl)isoxazole (44a). To a solution of Pd/C (50 mg, 10% purity) in methanol (5 mL) was added 5-cyclopropyl-3-(2,6-dichlorophenyl)-4-((piperidin-4-yloxy)methyl)isoxazole hydrochloride (1b) (500 mg, 1.24 mmol). The mixture was degassed and purged with H₂ 3 times, then stirred at 15° C. for 4 hours under H₂ ballon. The reaction mixture was filtered through a Celite pad and the filter cake was rinsed with methanol (10 mL). The combined filtrate was concentrated under reduced pressure. The residue was purified by prep-HPLC (HCl condition; column: Phenomenex luna C18 250*50 mm*10 um; mobile phase: [water (0.05% HCl)-ACN]; B %: 10%-30%, 10 min) to give 44a. MS mass calculated for [M+H]⁺(C₁₈H₂₁ClN₂O₂) requires m/z, 333.1/335.1, LCMS found m/z, 333.1/335.1; ¹H NMR (400 MHz, CHLOROFORM-d) δ=9.36 (br s, 1H), 7.55-7.49 (m, 1H), 7.49-7.43 (m, 1H), 7.42-7.35 (m, 2H), 4.35 (s, 2H), 3.57 (br s, 1H), 3.08-2.96 (m, 2H), 2.89 (br s, 2H), 2.10-2.04 (m, 1H), 2.02-1.87 (m, 2H), 1.76 (br d, J=11.5 Hz, 2H), 1.31-1.22 (m, 2H), 1.15-1.06 (m, 2H)

4-(4-((3-(2-chlorophenyl)-5-cyclopropylisoxazol-4-yl)methoxy)piperidin-1-yl)benzonitrile (44b). To a solution of 3-(2-chlorophenyl)-5-cyclopropyl-4-((piperidin-4-yloxy)methyl)isoxazole (44a) (100 mg, 300.46 umol) and 4-fluorobenzonitrile (2a) (145.56 mg, 1.20 mmol) in DMSO (2 mL) was added K₂CO₃ (166.10 mg, 1.20 mmol) in a sealed tube. The mixture was heated to 80° C. and stirred for 24 hours and was diluted with H₂O (10 mL) and extracted with ethyl acetate (20 mL*2). The combined organic phase was washed with brine (10 mL*2), dried over anhydrous Na₂SO₄, filtered and the filtrate was concentrated under reduced pressure. The residue was purified by prep-TLC (SiO₂, petroleum ether:ethyl acetate=1:1) to give 44b. MS mass calculated for [M+H]⁺(C₂₅H₂₄ClN₃O₂) requires m/z, 434.2/436.2, LCMS found m/z, 434.0/436.0; ¹H NMR (400 MHz, CHLOROFORM-d) δ=7.54-7.29 (m, 6H), 6.80 (d, J=8.8 Hz, 2H), 4.40 (s, 2H), 3.55-3.33 (m, 3H), 3.05 (ddd, J=3.7, 8.6, 12.7 Hz, 2H), 2.14 (tt, J=5.3, 8.4 Hz, 1H), 1.85-1.72 (m, 2H), 1.64-1.47 (m, 2H), 1.32-1.21 (m, 2H), 1.16-1.04 (m, 2H)

(Z)-4-(4-((3-(2-chlorophenyl)-5-cyclopropylisoxazol-4-yl)methoxy)piperidin-1-yl)-N′-hydroxybenzimidamide (44c). To a solution of 4-(4-((3-(2-chlorophenyl)-5-cyclopropylisoxazol-4-yl)methoxy)piperidin-1-yl)benzonitrile (44b) (0.07 g, 161.32 umol) in ethanol (1 mL) was added hydroxylamine (0.5 mL, 50% in water) and the mixture was stirred at 80° C. for 2 hours. The reaction mixture was diluted with water (5 mL) and extracted with dichloromethane (10 mL). The organic phase was washed with brine (5 mL), dried over anhydrous Na₂SO₄, filtered and the filtrate was concentrated. The residue was triturated with petroleum ether (5 mL) at 15° C. for 10 minutes and filtered. The solid collected was dried in vacuum to give 44c. MS mass calculated for [M+H]⁺(C₂₅H₂₇ClN₄O₃) requires m/z, 467.2/469.2, LCMS found m/z, 467.1/469.1; ¹H NMR (400 MHz, CHLOROFORM-d) δ=7.53-7.30 (m, 6H), 6.86 (d, J=8.8 Hz, 2H), 4.81 (br s, 2H), 4.40 (s, 2H), 3.48-3.32 (m, 3H), 2.92 (ddd, J=3.1, 9.1, 12.5 Hz, 2H), 2.21-2.09 (m, 1H), 1.81 (br d, J=11.8 Hz, 2H), 1.64-1.51 (m, 2H), 1.29-1.21 (m, 2H), 1.16-1.06 (m, 2H).

3-(4-(4-((3-(2-chlorophenyl)-5-cyclopropylisoxazol-4-yl)methoxy)piperidin-1-yl)phenyl)-1,2,4-oxadiazol-5(4H)-one (Compound 44). To a solution of (Z)-4-(4-((3-(2-chlorophenyl)-5-cyclopropylisoxazol-4-yl)methoxy)piperidin-1-yl)-N′-hydroxybenzimidamide (44c) (0.06 g, 128.49 umol) in ethanol (1 mL) was added diethyl carbonate (910.72 mg, 7.71 mmol, 934.07 uL) and CH₃ONa (138.82 mg, 770.95 umol, 30% in MeOH) in a sealed tube. The mixture was heated to 100° C. and stirred for 2 hours. The reaction mixture was diluted with water (5 mL) and extracted with dichloromethane (10 mL). The organic phase was washed with brine (5 mL), dried over anhydrous Na₂SO₄, filtered and the filtrate was concentrated under reduced pressure. The residue was purified by prep-HPLC (neutral condition; column: Waters Xbridge BEH C18 100*25 mm*5 um; mobile phase: [water (10 mM NH₄HCO₃)-ACN]; B %: 25%-55%, 10 min) to give Compound 44 (31.90 mg, 64.03 umol, 49.83% yield, 98.95% purity) as a white solid. MS mass calculated for [M+H]⁺(C₂₆H₂₅ClN₄O₄) requires m/z, 493.2/495.2, LCMS found m/z, 493.2/495.2; ¹H NMR (400 MHz, CHLOROFORM-d) δ=7.60 (d, J=8.8 Hz, 2H), 7.53-7.29 (m, 4H), 6.90 (d, J=8.8 Hz, 2H), 4.41 (s, 2H), 3.56-3.37 (m, 3H), 3.12-2.98 (m, 2H), 2.20-2.11 (m, 1H), 1.86-1.75 (m, 2H), 1.63-1.51 (m, 2H), 1.29-1.21 (m, 2H), 1.16-1.08 (m, 2H).

### Example 45

### 3-(5-(4-((5-cyclopropyl-3-(2,6-dichlorophenyl)isoxazol-4-yl)methoxy)piperidin-1-yl)-1H-1,2,4-triazol-3-yl)-1,2,4-oxadiazol-5(4H)-one

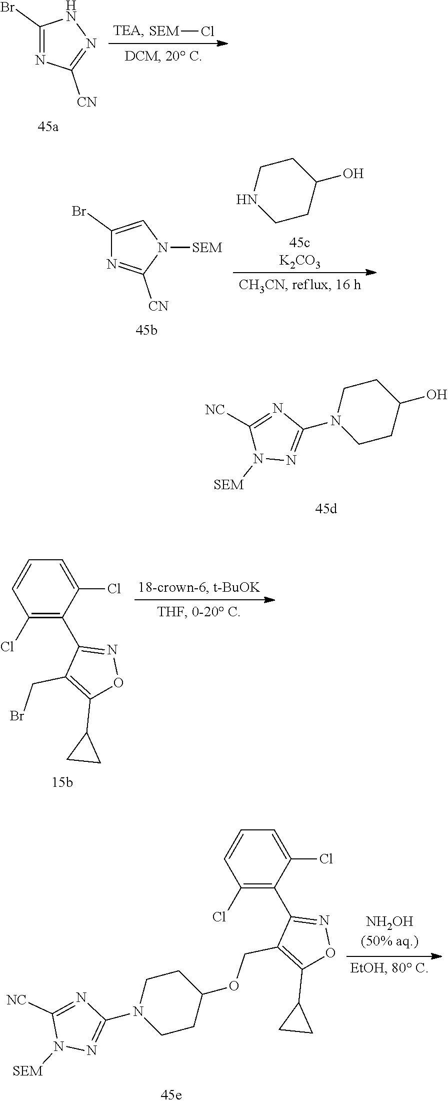

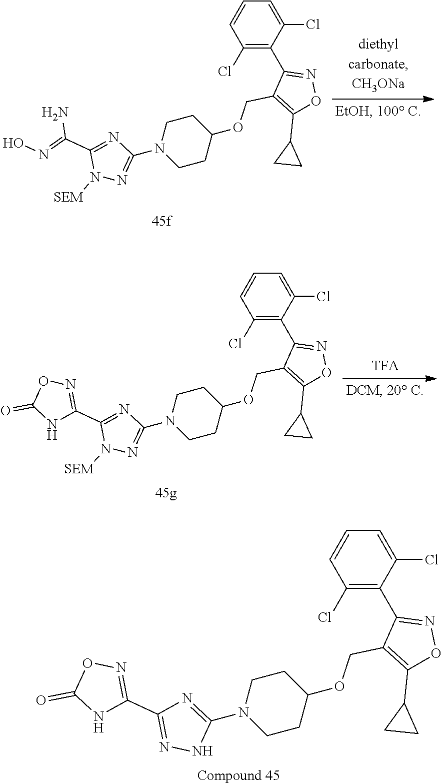 [SMILES: C[Si](C)(C)CCOCn1nc(N2CCC(OCc3c(-c4c(Cl)cccc4Cl)noc3C3CC3)CC2)nc1/C(N)=N/O ; C[Si](C)(C)CCOCn1nc(N2CCC(OCc3c(-c4c(Cl)cccc4Cl)noc3C3CC3)CC2)nc1-c1noc(=O)[nH]1 ; O=c1[nH]c(-c2n[nH]c(N3CCC(OCc4c(-c5c(Cl)cccc5Cl)noc4C4CC4)CC3)n2)no1]

3-bromo-1-((2-(trimethylsilyl)ethoxy)methyl)-1H-1,2,4-triazole-5-carbonitrile (45b). To a solution of 5-bromo-1H-1,2,4-triazole-3-carbonitrile (45a) (2 g, 11.56 mmol) in dichloromethane (40 mL) was added TEA (1.76 g, 17.34 mmol, 2.41 mL) and SEM-Cl (2.02 g, 12.14 mmol, 2.15 mL) at 20° C. The mixture was stirred at 20° C. for 0.5 hour. The reaction mixture was poured into H₂O (15 mL) and the mixture was extracted with dichloromethane (30 mL*2). The combined organic layers were washed with brine (10 mL), dried over anhydrous Na₂SO₄, filtered and the filtrate was concentrated under reduced pressure to give a residue. The residue was purified by column chromatography (SiO₂, petroleum ether:ethyl acetate=50:1 to 20:1) to give 45b. ¹H NMR (400 MHz, CHLOROFORM-d) δ=5.66-5.53 (m, 2H), 3.76-3.64 (m, 2H), 1.02-0.91 (m, 2H), 0.09-−0.02 (m, 9H).

3-(4-hydroxypiperidin-1-yl)-1-((2-(trimethylsilyl)ethoxy)methyl)-1H-1,2,4-triazole-5-carbonitrile (45d). To a solution of 3-bromo-1-((2-(trimethylsilyl)ethoxy)methyl)-1H-1,2,4-triazole-5-carbonitrile (45b) (1.36 g, 4.49 mmol) and piperidin-4-ol (45c) (907.29 mg, 8.97 mmol) in CH₃CN (10 mL) was added K₂CO₃ (1.86 g, 13.46 mmol) at 20° C. The mixture was heated to reflux for 16 hours and was poured into H₂O (15 mL). The resulting mixture was extracted with ethyl acetate (15 mL*3). The combined organic layer was washed with brine (10 mL), dried over anhydrous Na₂SO₄, filtered and the filtrate was concentrated under reduced pressure to give a residue. The residue was purified by column chromatography (SiO₂, petroleum ether:ethyl acetate=50:1 to 3:1) to give 45d. MS mass calculated for [M+H]⁺(C₁₄H₂₅N₅O₂Si) requires m/z, 324.2, LCMS found m/z, 324.1; ¹H NMR (400 MHz, CHLOROFORM-d) δ=5.33 (s, 2H), 3.94 (dt, J=4.0, 8.4 Hz, 1H), 3.80-3.71 (m, 4H), 3.22 (ddd, J=3.1, 9.5, 13.0 Hz, 2H), 2.07-1.96 (m, 2H), 1.75-1.64 (m, 2H), 0.98-0.90 (m, 2H), 0.05-0.00 (m, 9H).

3-(4-((5-cyclopropyl-3-(2,6-dichlorophenyl)isoxazol-4-yl)methoxy)piperidin-1-yl)-1-((2-(trimethylsilyl)ethoxy)methyl)-1H-1,2,4-triazole-5-carbonitrile (45e). To a solution of 3-(4-hydroxypiperidin-1-yl)-1-((2-(trimethylsilyl)ethoxy)methyl)-1H-1,2,4-triazole-5-carbonitrile (45d) (350 mg, 1.08 mmol) and 4-(bromomethyl)-5-cyclopropyl-3-(2,6-dichlorophenyl)isoxazole (15b) (350 mg, 1.08 mmol) in THE (20 mL) was added 18-CROWN-6 (429.00 mg, 1.62 mmol) and t-BuOK (1 M solution in THF, 1.62 mL) at 0° C. and the mixture was stirred at 20° C. for 2 hours. The reaction mixture was poured into water (10 mL) and extracted with ethyl acetate (20 mL*2). The combined organic layer was washed with brine (10 mL), dried over anhydrous Na₂SO₄, filtered and the filtrate was concentrated under reduced pressure to give a residue. The residue was purified by column chromatography (SiO₂, petroleum ether:ethyl acetate=50:1 to 3:1) to give 45e. MS mass calculated for [M+H]⁺(C₂₇H₃₄Cl₂N₆O₃Si) requires m/z, 589.2/591.2, LCMS found m/z, 589.2/591.2.

(Z)-3-(4-((5-cyclopropyl-3-(2,6-dichlorophenyl)isoxazol-4-yl)methoxy)piperidin-1-yl)-N′-hydroxy-1-((2-(trimethylsilyl)ethoxy)methyl)-1H-1,2,4-triazole-5-carboximidamide (45f). To a solution of 3-(4-((5-cyclopropyl-3-(2,6-dichlorophenyl)isoxazol-4-yl)methoxy)piperidin-1-yl)-1-((2-(trimethylsilyl)ethoxy)methyl)-1H-1,2,4-triazole-5-carbonitrile (45e) (350 mg, 593.64 umol) in ethanol (10 mL) was added hydroxylamine (0.7 mL, 50% in water) at 20° C. and the mixture was heated to 80° C. for 0.5 hour. The reaction mixture was filtered, and the filtrate was poured into H₂O (10 mL). The resulting mixture was extracted with ethyl acetate (20 mL*2). The combined organic layer was washed with brine (10 mL), dried over anhydrous Na₂SO₄, filtered and the filtrate was concentrated under reduced pressure to give a residue. The residue was purified by prep-TLC (SiO₂, dichloromethane:Methanol=10:1) to give 45f MS mass calculated for [M+H]⁺(C₂₇H₃₇Cl₂N₇O₄Si) requires m/z, 622.2/624.2, LCMS found m/z, 622.3/624.2; ¹H NMR (400 MHz, CHLOROFORM-d) δ=7.46-7.39 (m, 2H), 7.37-7.31 (m, 1H), 6.44 (br s, 1H), 5.26 (s, 2H), 5.10 (s, 2H), 4.33 (s, 2H), 3.80-3.71 (m, 2H), 3.56-3.47 (m, 2H), 3.40 (tt, J=3.8, 7.8 Hz, 1H), 3.08 (ddd, J=2.9, 9.2, 12.6 Hz, 2H), 2.20-2.12 (m, 1H), 1.79 (ddd, J=3.1, 6.3, 9.5 Hz, 2H), 1.59-1.48 (m, 2H), 1.31-1.24 (m, 2H), 1.17-1.10 (m, 2H), 0.96-0.88 (m, 2H), 0.00 (s, 9H).

3-(3-(4-((5-cyclopropyl-3-(2,6-dichlorophenyl)isoxazol-4-yl)methoxy)piperidin-1-yl)-1-((2-(trimethylsilyl)ethoxy)methyl)-1H-1,2,4-triazol-5-yl)-1,2,4-oxadiazol-5(4H)-one (45g). To a solution of (Z)-3-(4-((5-cyclopropyl-3-(2,6-dichlorophenyl)isoxazol-4-yl)methoxy)piperidin-1-yl)-N′-hydroxy-1-((2-(trimethylsilyl)ethoxy)methyl)-1H-1,2,4-triazole-5-carboximidamide (45f) (200 mg, 321.22 umol) in ethanol (5 mL) was added diethyl carbonate (1.95 g, 16.51 mmol, 2.00 mL) and CH₃ONa (289.23 mg, 1.61 mmol, 30% in MeOH) in a sealed tube at 20° C. The mixture was heated to 100° C. for 1 hour. and was poured into H₂O (10 mL). The mixture was extracted with ethyl acetate (15 mL*3). The combined organic layer was washed with brine (10 mL), dried over anhydrous Na₂SO₄, filtered and the filtrate was concentrated under reduced pressure to give a residue. The residue was purified by prep-TLC (SiO₂, dichloromethane:Methanol=10:1) to give 45g. MS mass calculated for [M+H]⁺(C₂₈H₃₅Cl₂N₇O₅Si) requires m/z, 648.2/650.2, LCMS found m/z, 648.2/650.1; ¹H NMR (400 MHz, CHLOROFORM-d) δ=8.87 (br s, 1H), 7.48-7.39 (m, 2H), 7.38-7.32 (m, 1H), 5.31 (s, 2H), 4.34 (s, 2H), 3.75 (br t, J=8.0 Hz, 2H), 3.51 (br s, 2H), 3.45 (br s, 1H), 3.24-3.12 (m, 2H), 2.20-2.11 (m, 1H), 1.78 (br s, 2H), 1.56 (br d, J=8.6 Hz, 2H), 1.28 (br s, 2H), 1.21-1.10 (m, 2H), 0.93 (br t, J=8.2 Hz, 2H), 0.01 (s, 9H)

3-(5-(4-((5-cyclopropyl-3-(2,6-dichlorophenyl)isoxazol-4-yl)methoxy)piperidin-1-yl)-1H-1,2,4-triazol-3-yl)-1,2,4-oxadiazol-5(4H)-one (Compound 45). To a solution of 3-(3-(4-((5-cyclopropyl-3-(2,6-dichlorophenyl)isoxazol-4-yl)methoxy)piperidin-1-yl)-1-((2-(trimethylsilyl)ethoxy)methyl)-1H-1,2,4-triazol-5-yl)-1,2,4-oxadiazol-5(4H)-one (45g) (60 mg, 92.51 umol) in dichloromethane (2 mL) was added TFA (924.00 mg, 8.10 mmol, 0.6 mL) at 20° C. and the mixture was stirred at 20° C. for 6 hours. The reaction mixture was concentrated under reduced pressure to remove dichloromethane. The residue was purified by prep-TLC (SiO₂, dichloromethane:Methanol=10:1) to give Compound 45. MS mass calculated for [M+H]⁺(C₂₂H₂₁Cl₂N₇O₄) requires m/z, 518.1/520.1, LCMS found m/z, 518.1/520.1; ¹H NMR (400 MHz, CHLOROFORM-d) δ=12.59 (br s, 1H), 10.98 (br s, 1H), 7.41 (br s, 2H), 7.35 (br s, 1H), 4.35 (br s, 2H), 3.53 (br s, 1H), 3.46 (br s, 2H), 3.30 (br s, 2H), 2.21-2.08 (m, 1H), 1.73 (br s, 2H), 1.59 (br s, 2H), 1.26 (br s, 2H), 1.15 (br d, J=5.7 Hz, 2H); ¹H NMR (400 MHz, DMSO-d6) δ=13.21 (br s, 1H), 12.97 (br s, 1H), 7.60 (br d, J=7.2 Hz, 2H), 7.52 (br d, J=7.1 Hz, 1H), 4.30 (br s, 2H), 3.50-3.34 (m, 4H), 3.10 (br s, 1H), 2.33 (br s, 1H), 1.65 (br s, 2H), 1.30 (br s, 2H), 1.14 (br d, J=8.1 Hz, 2H), 1.09 (br s, 2H).

### Example 46

### 6-(5-(4-((5-cyclopropyl-3-(2,6-dichlorophenyl)isoxazol-4-yl)methoxy)piperidin-1-yl)pyridin-2-yl)-1,2,4-triazine-3, 5(2H,4H)-dione

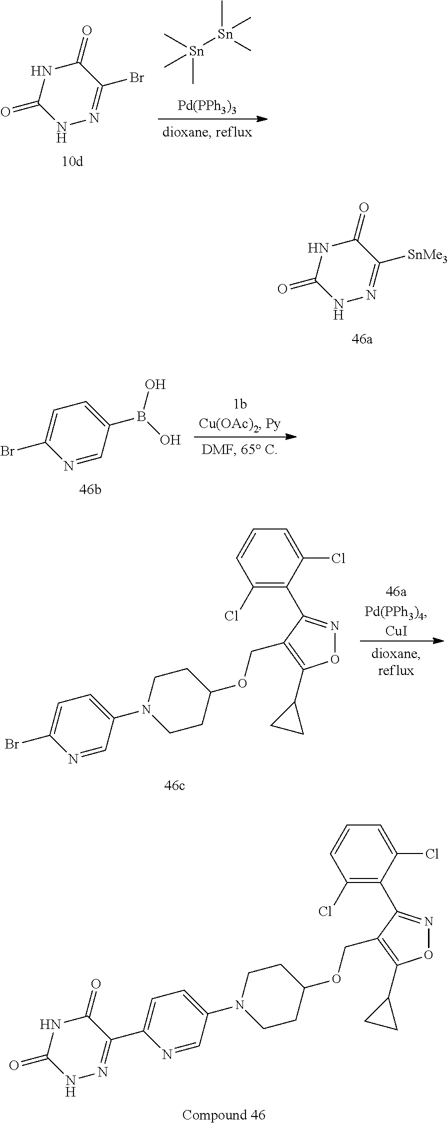

6-(trimethylstannyl)-1,2,4-triazine-3,5(2H,4H)-dione (46a). To a mixture of 6-bromo-1,2,4-triazine-3,5(2H,4H)-dione (10d) (1 g, 5.21 mmol) and trimethyl(trimethylstannyl)stannane (2.56 g, 7.81 mmol, 1.62 mL) in 1,4-dioxane (100 mL) was added Pd(PPh₃)₄ (300.97 mg, 260.46 umol) at 20° C. under N₂. The mixture was heated to reflux and stirred for 2 hours. The reaction mixture was concentrated under reduced pressure. The residue was purified by column chromatography (SiO₂, petroleum ether:ethyl acetate=20:1 to 10:1) to give 46a. MS mass calculated for [M+H]⁺(C₆H₁₁N₃O₂Sn) requires m/z, 278.0/276.0, LCMS found m/z, 278.0/276.0; ¹H NMR (400 MHz, DMSO-d6) δ=12.43 (s, 1H), 11.67 (s, 1H), 2.38-2.27 (m, 9H).

4-(((1-(6-bromopyridin-3-yl)piperidin-4-yl)oxy)methyl)-5-cyclopropyl-3-(2,6-dichlorophenyl)isoxazole (46c). To a mixture of 5-cyclopropyl-3-(2,6-dichlorophenyl)-4-((piperidin-4-yloxy)methyl)isoxazole hydrochloride (1b) (500 mg, 1.36 mmol) and (6-bromopyridin-3-yl)boronic acid (46b) (274.75 mg, 1.36 mmol) in DMF (15 mL) was added Cu(OAc)₂ (296.73 mg, 1.63 mmol) and pyridine (215.37 mg, 2.72 mmol, 219.77 uL) at 20° C. The mixture was stirred at 65° C. for 12 hours and was poured into ethyl acetate (20 mL) and water (10 mL). The phases were separated and the aqueous phase was washed with water (10 mL*2). The combined organic phase was washed with brine (20 mL), dried over anhydrous Na₂SO₄, filtered and the filtrate was concentrated under reduced pressure. The residue was purified by column chromatography (SiO₂, petroleum ether:ethyl acetate=20:1 to 5:1) to give 46c. ¹H NMR (400 MHz, CHLOROFORM-d) δ=7.95 (d, J=3.1 Hz, 1H), 7.41-7.36 (m, 2H), 7.32-7.27 (m, 2H), 7.03 (dd, J=3.1, 8.7 Hz, 1H), 4.34 (s, 2H), 3.51-3.39 (m, 1H), 3.17 (ddd, J=3.7, 7.8, 11.9 Hz, 2H), 2.91 (ddd, J=3.5, 8.2, 12.2 Hz, 2H), 2.21-2.10 (m, 1H), 1.84-1.72 (m, 2H), 1.65-1.57 (m, 2H), 1.29-1.26 (m, 2H), 1.16-1.09 (m, 2H).

6-(5-(4-((5-cyclopropyl-3-(2,6-dichlorophenyl)isoxazol-4-yl)methoxy)piperidin-1-yl)pyridin-2-yl)-1,2,4-triazine-3,5(2H,4H)-dione (Compound 46). To a mixture of 4-(((1-(6-bromopyridin-3-yl)piperidin-4-yl)oxy)methyl)-5-cyclopropyl-3-(2,6-dichlorophenyl)isoxazole (46c) (80 mg, 152.89 umol) and 6-(trimethylstannyl)-1,2,4-triazine-3,5(2H,4H)-dione (46a) (84.36 mg, 305.78) in 1,4-dioxane (5 mL) was added Pd(PPh₃)₄(17.67 mg, 15.29 umol) and CuI (29.12 mg, 152.89 umol) at 20° C. under N₂. The mixture was stirred at 120° C. for 12 hours and was concentrated under reduced pressure. The residue was purified by prep-HPLC (TFA condition), then repurified by prep-HPLC (neutral condition; column: Phenomenex Gemini-NX C18 75*30 mm*3 um; mobile phase: [water (10 mM NH₄HCO₃)-ACN]; B %: 30%-60%, 8 min) to afford Compound 46. MS mass calculated for [M+H]⁺(C₂₆H₂₄Cl₂N₆O₄) requires m/z, 555.1/557.1, LCMS found m/z, 555.2/557.2; MS mass calculated for [M−H]⁻ (C₂₆H₂₄Cl₂N₆O₄) requires m/z, 553.1/555.1, LCMS found m/z, 553.2/555.2; ¹H NMR (400 MHz, DMSO-d6) δ=8.73 (br s, 2H), 8.28 (br s, 1H), 7.71 (br d, J=8.8 Hz, 1H), 7.64-7.55 (m, 2H), 7.53-7.44 (m, 1H), 7.32-7.23 (m, 1H), 4.32 (s, 2H), 3.41 (br d, J=3.4 Hz, 1H), 3.31 (br s, 2H), 2.98 (br t, J=9.0 Hz, 2H), 2.41-2.28 (m, 1H), 1.70 (br s, 2H), 1.34 (br d, J=8.8 Hz, 2H), 1.20-1.12 (m, 2H), 1.12-1.02 (m, 2H).

### Example 47

### 3-(4-(4-((5-cyclopropyl-3-(2-(trifluoromethoxy)phenyl)isoxazol-4-yl)methoxy)azepan-1-yl)phenyl)-1,2,4-oxadiazol-5(4H)-one

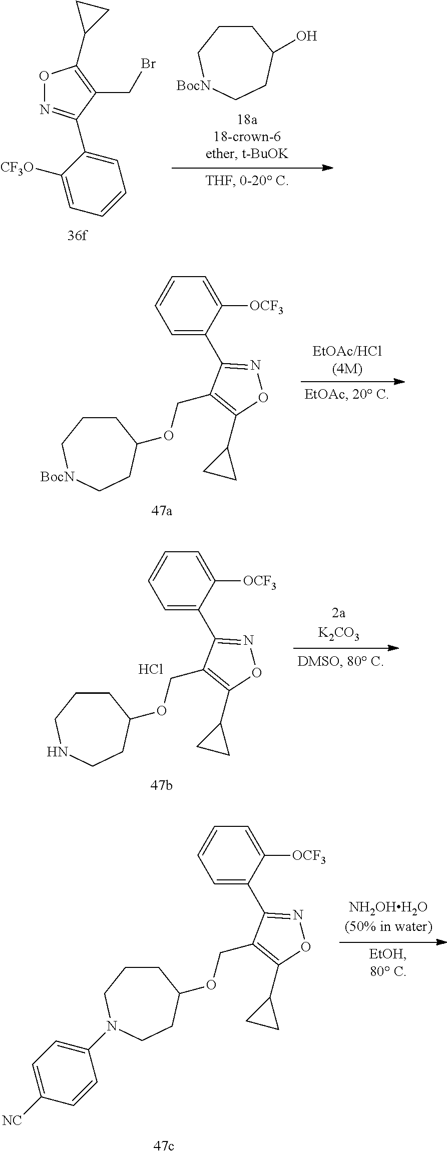

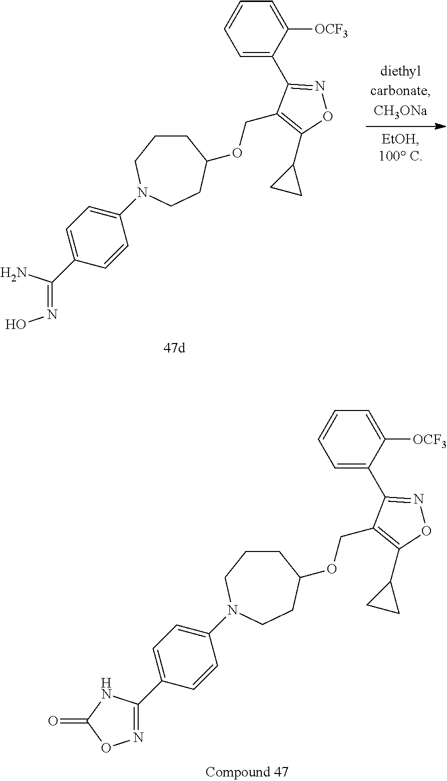 [SMILES: N/C(=N\O)c1ccc(N2CCCC(OCc3c(-c4ccccc4OC(F)(F)F)noc3C3CC3)CC2)cc1 ; O=c1[nH]c(-c2ccc(N3CCCC(OCc4c(-c5ccccc5OC(F)(F)F)noc4C4CC4)CC3)cc2)no1]

Tert-butyl 4-((5-cyclopropyl-3-(2-(trifluoromethoxy)phenyl)isoxazol-4-yl)methoxy)azepane-1-carboxylate (47a). To a solution of 18-CROWN-6 (164.22 mg, 621.30 umol) and tert-butyl 4-hydroxyazepane-1-carboxylate (18a) (115.93 mg, 538.46 umol) in THF (5 mL) was added t-BuOK (1 M solution in THF, 621.30 uL) dropwise at 0° C. After stirring at 20° C. for 0.5 hour 4-(bromomethyl)-5-cyclopropyl-3-(trifluoromethoxy)phenyl)isoxazole (36f) (150 mg, 414.20 umol) was added, the mixture was stirred at 20° C. for 1.5 hours. The reaction mixture was poured into water (10 mL) and extracted with ethyl acetate (20 mL*1, 10 mL*2). The combined organic layer was washed with brine (20 mL), dried over anhydrous Na₂SO₄, filtered and the filtrate was concentrated to give the residue, which was purified by prep-TLC (petroleum ether:ethyl acetate=3:1) to give 47a. ¹H NMR (CHLOROFORM-d, 400 MHz): δ=7.59-7.48 (m, 2H), 7.42-7.36 (m, 2H), 4.38-4.26 (m, 2H), 3.53-3.30 (m, 3H), 3.26-3.11 (m, 2H), 2.16-2.08 (m, 1H), 1.83-1.69 (m, 2H), 1.61 (br d, J=2.9 Hz, 3H), 1.49 (br s, 1H), 1.44 (s, 9H), 1.26-1.20 (m, 2H), 1.17-1.07 (m, 2H).

4-((azepan-4-yloxy)methyl)-5-cyclopropyl-3-(2-trifluoromethoxy)phenyl)isoxazole (47b). To a solution of tert-butyl 4-((5-cyclopropyl-3-(2-(trifluoromethoxy)phenyl)isoxazol-4-yl)methoxy)azepane-1-carboxylate (47a) (170 mg, 342.38 umol) in ethyl acetate (2 mL) was added HCl/ethyl acetate (2 mL, 4M) at 20° C. for 2 hours. The reaction mixture was concentrated under reduced pressure to give 47b.

4-(4-((5-cyclopropyl-3-(2-(trifluoromethoxy)phenyl)isoxazol-4-yl)methoxy)azepan-1-yl)benzonitrile (47c). To a solution of 4-((azepan-4-yloxy)methyl)-5-cyclopropyl-3-(2-(trifluoromethoxy)phenyl)isoxazole hydrochloride (47b) (140 mg, 323.43 umol) and 4-fluorobenzonitrile (2a) (195.85 mg, 1.62 mmol) in DMSO (5 mL) was added K₂CO₃ (178.80 mg, 1.29 mmol) at 20° C. and the mixture was stirred at 80° C. for 16 hours. The reaction mixture was diluted with ethyl acetate (20 mL) and washed with brine (10 mL*2, 5 mL*2). The combined organic layer was dried over anhydrous Na₂SO₄, filtered and the filtrate was concentrated. The residue was purified by prep-TLC (petroleum ether:ethyl acetate=2:1) to give 47c. MS mass calculated for [M+H]⁺(C₂₇H₂₆F₃N₃O₃) requires m/z, 498.2, LCMS found m/z, 498.2; ¹H NMR (CHLOROFORM-d, 400 MHz): δ=7.57-7.49 (m, 2H), 7.44 (d, J=9.0 Hz, 2H), 7.41-7.35 (m, 2H), 6.60 (d, J=9.0 Hz, 2H), 4.39-4.29 (m, 2H), 3.52-3.39 (m, 3H), 3.36 (t, J=5.6 Hz, 2H), 3.32-3.24 (m, 1H), 2.14-2.05 (m, 1H), 1.87-1.78 (m, 2H), 1.77-1.63 (m, 3H), 1.24-1.20 (m, 2H), 1.11-1.06 (m, 2H).

(Z)-4-(4-((5-cyclopropyl-3-(2-(trifluoromethoxy)phenyl)isoxazol-4-yl)methoxy)azepan-1-yl)-N′-hydroxybenzimidamide (47d). To a solution of 4-(4-((5-cyclopropyl-3-(2-(trifluoromethoxy)phenyl)isoxazol-4-yl)methoxy)azepan-1-yl)-benzonitrile (47c) (50 mg, 100.50 umol) in ethanol (6 mL) was added hydroxylamine (1 mL, 50% in water) at 20° C. and the mixture was stirred at 80° C. for 4 hours. The reaction mixture was concentrated, and the residue was diluted with ethyl acetate (15 mL) and washed with brine (5 mL*2). The organic layer was dried over anhydrous Na₂SO₄, filtered and the filtrate was concentrated. The residue was purified by prep-TLC (dichloromethane:methanol=10:1) to give 47d. MS mass calculated for [M+H]⁺(C₂₇H₂₉F₃N₄O₄) requires m/z, 531.2, LCMS found m/z, 531.2; ¹H NMR (CHLOROFORM-d, 400 MHz): δ=7.58-7.43 (m, 4H), 7.42-7.35 (m, 2H), 6.62 (br d, J=7.9 Hz, 2H), 4.80 (br s, 2H), 4.39-4.28 (m, 2H), 3.49-3.40 (m, 2H), 3.35 (br t, J=5.4 Hz, 2H), 3.30-3.22 (m, 1H), 2.15-2.07 (m, 1H), 1.83 (br dd, J=6.4, 19.3 Hz, 2H), 1.77-1.63 (m, 4H), 1.25-1.19 (m, 2H), 1.12-1.06 (m, 2H).

3-(4-(4-((5-cyclopropyl-3-(2-(trifluoromethoxy)phenyl)isoxazol-4-yl)methoxy)azepan-1-yl)phenyl)-1,2,4-oxadiazol-5(4H)-one (Compound 47). To a solution of (Z)-4-(4-((5-cyclopropyl-3-(2-(trifluoromethoxy)phenyl)isoxazol-4-yl)methoxy)azepan-1-yl)-N′-hydroxybenzimidamide (47d) (50 mg, 94.24 umol) and diethyl carbonate (975.00 mg, 8.25 mmol, 1 mL) in ethanol (4 mL) was added CH₃ONa (169.70 mg, 942.44 umol, 0.2 mL, 30% in MeOH) at 20° C. in a sealed tube and the mixture was stirred at 100° C. for 5 hours. he reaction mixture was concentrated under reduced pressure to remove solvent. Water (5 mL) and ethyl acetate (5 mL) were added into the reaction mixture and the phases were separated. The aqueous phase was extracted with ethyl acetate (5 mL*4). The combined organic phase was washed with brine (20 mL*2), dried with anhydrous Na₂SO₄, filtered and the filtrate was concentrated under reduced pressure which was purified by prep-HPLC (neutral condition: column: Waters Xbridge BEH C18 100*25 mm*5 um; mobile phase: [water(10 mM NH₄HCO₃)-ACN]; B %: 25%-55%, 10 min) to give Compound 47. MS mass calculated for [M+H]⁺(C₂₈H₂₇F₃N₄O₅) requires m/z, 557.2, LCMS found m/z, 557.2; ¹H NMR (CHLOROFORM-d, 400 MHz): δ=7.58 (d, J=9.0 Hz, 2H), 7.56-7.50 (m, 2H), 7.42-7.36 (m, 2H), 6.69 (d, J=9.0 Hz, 2H), 4.39-4.30 (m, 2H), 3.52-3.43 (m, 2H), 3.39 (br t, J=5.0 Hz, 2H), 3.35-3.27 (m, 1H), 2.15-2.07 (m, 1H), 1.90-1.80 (m, 2H), 1.79-1.64 (m, 3H), 1.58-1.50 (m, 1H), 1.25-1.20 (m, 2H), 1.13-1.07 (m, 2H).

### Example 48

### 5-(4-(4-((5-cyclopropyl-3-(2,6-dichlorophenyl)isoxazol-4-yl)methoxy)piperidin-1-yl)-2-fluorophenyl)-1,3,4-oxadiazol-2(3H)-one

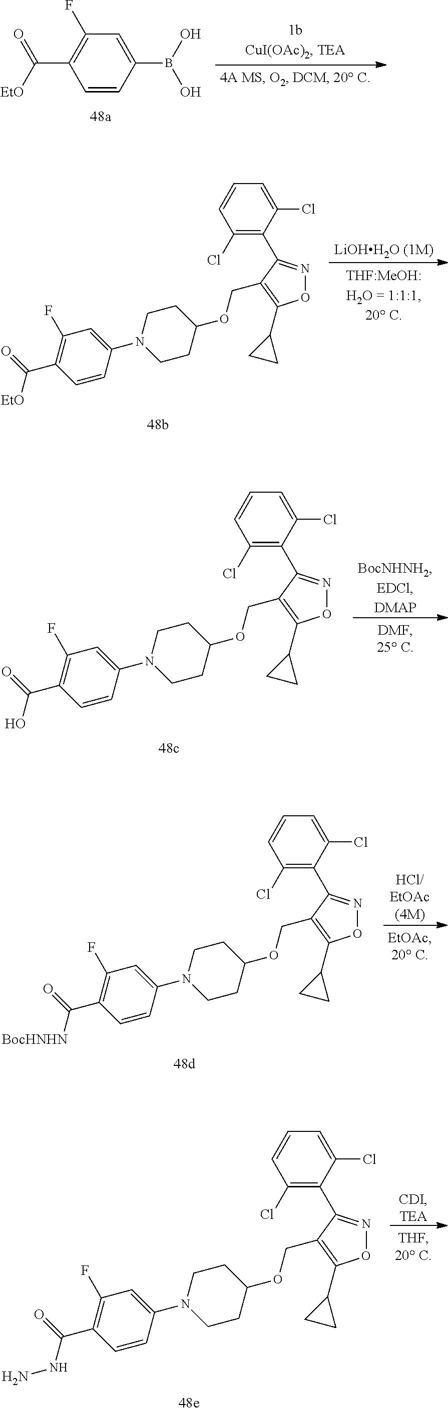

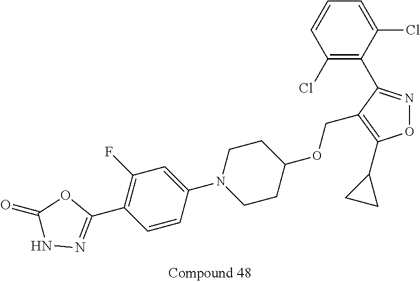 [SMILES: O=c1[nH]nc(-c2ccc(N3CCC(OCc4c(-c5c(Cl)cccc5Cl)noc4C4CC4)CC3)cc2F)o1]

Ethyl 4-(4-((5-cyclopropyl-3-(2,6-dichlorophenyl)isoxazol-4-yl)methoxy)piperidin-1-yl)-2-fluorobenzoate (48b). To a mixture of 5-cyclopropyl-3-(2,6-dichlorophenyl)-4-((piperidin-4-yloxy)methyl)isoxazole hydrochloride (1b) (300 mg, 816.84 umol) and (4-(ethoxycarbonyl)-3-fluorophenyl)boronic acid (48a) (207.79 mg, 980.21 umol) in dichloromethane (20 mL) was added Cu(OAc)₂ (148.37 mg, 816.84 umol) and TEA (247.97 mg, 2.45 mmol, 341.08 uL), molecular sieve 4 A (150 mg) at 15° C. The mixture was stirred at 20° C. for 12 hours under O₂ ballon and the reaction mixture was poured into water (20 mL) and extracted with ethyl acetate (20 mL*4). The combined organic phase was washed with brine (10 mL), dried with anhydrous Na₂SO₄, filtered and the filtrate was concentrated under reduced pressure. The residue was purified by column chromatography (SiO₂, petroleum ether:ethyl acetate=20:1 to 10:1) to give 48b. MS mass calculated for [M+H]⁺(C₂₇H₂₇Cl₂FN₂O₄) requires m/z, 533.1/535.1, LCMS found m/z, 533.2/535.0.

4-(4-((5-cyclopropyl-3-(2,6-dichlorophenyl)isoxazol-4-yl)methoxy)piperidin-1-yl)-2-fluorobenzoic acid (48c). To a mixture of ethyl 4-(4-((5-cyclopropyl-3-(2,6-dichlorophenyl)isoxazol-4-yl)methoxy)piperidin-1-yl)-2-fluorobenzoate (48b) (190 mg, 356.19 umol) in THF (1.5 mL), methanol (1.5 mL) and H₂O (1.5 mL) was added LiOH H₂O (1 M, 1.58 mL) at 20° C. under N₂. The mixture was stirred at 20° C. for 12 hours and was concentrated under N₂ to remove most of solvents. HCl (1 N) was added to the mixture at 20° C. to adjust pH=4-5 while white solid precipitated. The mixture was filtered, and filter cake was dried in vacuum to give 48c. MS mass calculated for [M+H]⁺(C₂₅H₂₃Cl₂FN₂O₄) requires m/z, 505.1/507.1, LCMS found m/z, 505.2/507.0.

Tert-butyl 2-(4-(4-((5-cyclopropyl-3-(2,6-dichlorophenyl)isoxazol-4-yl)methoxy)piperidin-1-yl)-2-fluorobenzoyl)hydrazinecarboxylate (48d). To a solution of 4-(4-((5-cyclopropyl-3-(2,6-dichlorophenyl)isoxazol-4-yl)methoxy)piperidin-1-yl)-2-fluorobenzoic acid (48c) (120 mg, 237.45 umol) and tert-butyl N-aminocarbamate (62.76 mg, 474.90 umol) in DMF (5 mL) were added EDCI (59.18 mg, 308.69 umol) and DMAP (580.18 ug, 4.75 umol) at 20° C. and the mixture was heated to 25° C. and stirred for 16 hours. The reaction mixture was poured into H₂O (10 mL), and the mixture was extracted with ethyl acetate (15 mL*3). The combined organic layer was washed with brine (10 mL), dried over anhydrous Na₂SO₄, filtered and the filtrate was concentrated under reduced pressure to give a residue. The residue was purified by prep-TLC (SiO₂, dichloromethane:methanol=10:1) to give 48d. MS mass calculated for [M+H]⁺(C₃₀H₃₃Cl₂FN₄O₅) requires m/z, 619.2/621.2, LCMS found m/z, 619.4/621.4.

4-(4-((5-cyclopropyl-3-(2,6-dichlorophenyl)isoxazol-4-yl)methoxy)piperidin-1-yl)-2-fluorobenzohydrazide (48e). To a solution of tert-butyl 2-(4-(4-((5-cyclopropyl-3-(2,6-dichlorophenyl)isoxazol-4-yl)methoxy)piperidin-1-yl)-2-fluorobenzoyl)hydrazinecarboxylate (48d) (50 mg, 80.71 umol) in ethyl acetate (2.5 mL) was added HCl/ethyl acetate (2.5 mL, 4 M) at 20° C. and the mixture was stirred at 20° C. for 4 hours. The reaction mixture was concentrated under reduced pressure to give 48e. MS mass calculated for [M+H]⁺(C₂₅H₂₅Cl₂FN₄O₃) requires m/z, 519.1/521.1, LCMS found m/z, 519.3/521.3.

5-(4-(4-((5-cyclopropyl-3-(2,6-dichlorophenyl)isoxazol-4-yl)methoxy)piperidin-1-yl)-2-fluorophenyl)-1,3,4-oxadiazol-2(3H)-one (Compound 48). To a solution of 4-(4-((5-cyclopropyl-3-(2,6-dichlorophenyl)isoxazol-4-yl)methoxy)piperidin-1-yl)-2-fluorobenzohydrazide hydrochloride (48e) (45 mg, 80.96 umol) in THF (5 mL) was added CDI (26.25 mg, 161.91 umol) and TEA (24.58 mg, 242.87 umol, 33.80 uL) at 20° C. and the mixture was stirred at 20° C. for 4 hours. The reaction mixture was poured into H₂O (10 mL) and was extracted with ethyl acetate (20 mL*2). The combined organic layer was washed with brine (10 mL), dried over anhydrous Na₂SO₄, filtered and the filtrate was concentrated under reduced pressure to give a residue. The residue was purified by prep-TLC (SiO₂, dichloromethane:methanol=10:1) to give Compound 48. MS mass calculated for [M+H]⁺(C₂₆H₂₃Cl₂FN₄O₄) requires m/z, 545.1/547.1, LCMS found m/z, 545.2/547.1; ¹H NMR (400 MHz, CHLOROFORM-d) δ=8.72 (br s, 1H), 7.58 (t, J=8.8 Hz, 1H), 7.44-7.36 (m, 2H), 7.35-7.28 (m, 1H), 6.64 (dd, J=2.4, 8.9 Hz, 1H), 6.56 (dd, J=2.3, 14.7 Hz, 1H), 4.35 (s, 2H), 3.50 (tt, J=3.5, 7.1 Hz, 1H), 3.38-3.28 (m, 2H), 3.13-3.03 (m, 2H), 2.20-2.10 (m, 1H), 1.76 (dt, J=3.8, 8.5 Hz, 2H), 1.59-1.50 (m, 2H), 1.32-1.25 (m, 2H), 1.18-1.10 (m, 2H).

### Example 49

### 3-(4-(4-((5-cyclopropyl-3-(2,6-difluorophenyl)isoxazol-4-yl)methoxy)azepan-1-yl)phenyl)-1,2,4-oxadiazol-5(4H)-one

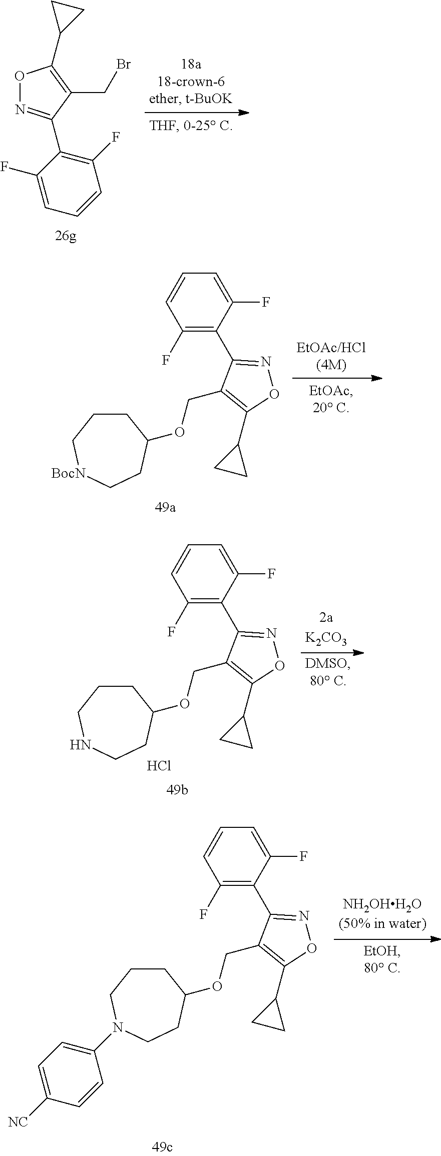 [SMILES: Fc1cccc(F)c1-c1noc(C2CC2)c1CBr ; CC(C)(C)OC(=O)N1CCCC(OCc2c(-c3c(F)cccc3F)noc2C2CC2)CC1 ; Fc1cccc(F)c1-c1noc(C2CC2)c1COC1CCCNCC1 ; Cl ; N#Cc1ccc(N2CCCC(OCc3c(-c4c(F)cccc4F)noc3C3CC3)CC2)cc1]

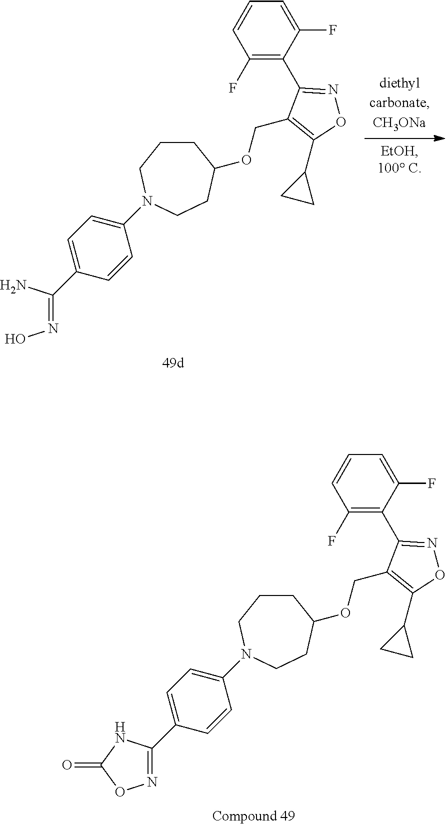 [SMILES: N/C(=N\O)c1ccc(N2CCCC(OCc3c(-c4c(F)cccc4F)noc3C3CC3)CC2)cc1 ; O=c1[nH]c(-c2ccc(N3CCCC(OCc4c(-c5c(F)cccc5F)noc4C4CC4)CC3)cc2)no1]

Tert-butyl 4-((5-cyclopropyl-3-(2,6-difluorophenyl)isoxazol-4-yl)methoxy)azepane-1-carboxylate (49a). To a solution of tert-butyl 4-hydroxyazepane-1-carboxylate (18a) (150.78 mg, 700.36 umol) in THF (5 mL) was added 18-CROWN-6 (277.68 mg, 1.05 mmol) and t-BuOK (1 M solution in THF, 1.05 mL) at 0° C. and the mixture was stirred at 20° C. for 30 minutes. 4-(Bromomethyl)-5-cyclopropyl-3-(2,6-difluorophenyl)isoxazole (26g) (220 mg, 700.36 umol) in THF (5 mL) was added to the mixture dropwise at 20° C. and the mixture was stirred at 20° C. for 1.5 hours. The reaction mixture was poured into H₂O (10 mL) and was extracted with ethyl acetate (20 mL*2). The combined organic layer was washed with brine (10 mL), dried over anhydrous Na₂SO₄, filtered and the filtrate was concentrated under reduced pressure to give a residue. The residue was purified by prep-TLC (SiO₂, petroleum ether:ethyl acetate=3:1) to give 49a. MS mass calculated for [M+H]⁺(C₂₄H₃₀F₂N₂O₄) requires m/z, 449.2, LCMS found m/z, 449.2; ¹H NMR (400 MHz, CHLOROFORM-d) δ=7.43 (quin, J=7.3 Hz, 1H), 7.02 (br t, J=7.6 Hz, 2H), 4.40-4.28 (m, 2H), 3.53-3.28 (m, 3H), 3.27-3.06 (m, 3H), 2.25-2.05 (m, 1H), 1.77-1.66 (m, 2H), 1.62-1.58 (m, 2H), 1.54 (br s, 1H), 1.49-1.45 (m, 1H), 1.44 (s, 9H), 1.28-1.20 (m, 2H), 1.12 (br d, J=7.9 Hz, 2H).

4-((azepan-4-yloxy)methyl)-5-cyclopropyl-3-(2,6-difluorophenyl)isoxazole (49b). To a solution of tert-butyl 4-((5-cyclopropyl-3-(2,6-difluorophenyl)isoxazol-4-yl)methoxy)azepane-1-carboxylate (49a) (200 mg, 445.93 umol) in ethyl acetate (2 mL) was added HCl/ethyl acetate (4 mL, 4 M) at 20° C. and the mixture was stirred at 20° C. for 1 hour. The reaction mixture was concentrated under reduced pressure to give 49b. MS mass calculated for [M+H]⁺(C₁₉H₂₂F₂N₂O₂) requires m/z, 349.2, LCMS found m/z, 349.1.

4-(4-((5-cyclopropyl-3-(2,6-difluorophenyl)isoxazol-4-yl)methoxy)azepan-1-yl)benzonitrile (49c). To a solution of 4-((azepan-4-yloxy)methyl)-5-cyclopropyl-3-(2,6-difluorophenyl)isoxazole hydrochloride (49b) (140 mg, 363.78 umol) in DMSO (5 mL) was added K₂CO₃ (251.38 mg, 1.82 mmol) and 4-fluorobenzonitrile (2a) (220.29 mg, 1.82 mmol) at 20° C. and the mixture was heated to 80° C. and stirred for 16 hours. The reaction mixture was poured into water (10 mL) and extracted with ethyl acetate (20 mL*2). The combined organic layer was washed with brine (10 mL), dried over anhydrous Na₂SO₄, filtered and the filtrate was concentrated under reduced pressure to give a residue. The residue was purified by prep-TLC (SiO₂, petroleum ether:ethyl acetate=2:1) to give 49c. MS mass calculated for [M+H]⁺(C₂₆H₂₅F₂N₃O₂) requires m/z, 450.2, LCMS found m/z, 450.2; ¹H NMR (400 MHz, CHLOROFORM-d) δ=7.48-7.39 (m, 3H), 7.06-6.98 (m, 2H), 6.59 (d, J=9.0 Hz, 2H), 4.40-4.29 (m, 2H), 3.52-3.38 (m, 2H), 3.38-3.32 (m, 2H), 3.32-3.23 (m, 1H), 2.10 (tt, J=5.0, 8.4 Hz, 1H), 1.87-1.58 (m, 5H), 1.54-1.45 (m, 1H), 1.30-1.20 (m, 2H), 1.14-1.06 (m, 2H).

(Z)-4-(4-((5-cyclopropyl-3-(2,6-difluorophenyl)isoxazol-4-yl)methoxy)azepan-1-yl)-N′-hydroxybenzimidamide (49d). To a solution of 4-(4-((5-cyclopropyl-3-(2,6-difluorophenyl)isoxazol-4-yl)methoxy)azepan-1-yl)benzonitrile (49c) (100 mg, 222.47 umol) in ethanol (5 mL) was added hydroxylamine (0.5 mL, 50% in water) at 20° C. and the mixture was heated to 80° C. for 1 hour. The reaction mixture was poured into H₂O (10 mL) and was extracted with ethyl acetate (20 mL*2). The combined organic layer was washed with brine (10 mL), dried over anhydrous Na₂SO₄, filtered and the filtrate was concentrated under reduced pressure to give a residue. The residue was purified by prep-TLC (SiO₂, dichloromethane:methanol=10:1) to give 49d. MS mass calculated for [M+H]⁺(C₂₆H₂₈F₂N₄O₃) requires m/z, 483.2, LCMS found m/z, 483.2; ¹H NMR (400 MHz, CHLOROFORM-d) δ=7.49-7.38 (m, 3H), 7.06-6.98 (m, 2H), 6.61 (d, J=9.0 Hz, 2H), 4.78 (br s, 2H), 4.38-4.29 (m, 2H), 3.48-3.39 (m, 2H), 3.34 (br t, J=4.1 Hz, 2H), 3.30-3.21 (m, 1H), 2.16-2.07 (m, 1H), 1.90-1.75 (m, 2H), 1.75-1.65 (m, 2H), 1.63-1.50 (m, 2H), 1.26-1.20 (m, 2H), 1.13-1.05 (m, 2H).

3-(4-(4-((5-cyclopropyl-3-(2,6-difluorophenyl)isoxazol-4-yl)methoxy)azepan-1-yl)phenyl)-1,2,4-oxadiazol-5(4H)-one (Compound 49). To a solution of (Z)-4-(4-((5-cyclopropyl-3-(2,6-difluorophenyl)isoxazol-4-yl)methoxy)azepan-1-yl)-N′-hydroxybenzimidamide (49d) (50 mg, 103.62 umol) in ethanol (2.5 mL) was added diethyl carbonate (487.50 mg, 4.13 mmol, 0.5 mL) and CH₃ONa (93.30 mg, 518.11 umol, 30% in MeOH) at 20° C. and the mixture was heated to 100° C. for 0.5 hour. The reaction mixture was poured into H₂O (10 mL) and was extracted with ethyl acetate (20 mL*2). The combined organic layer was washed with brine (10 mL), dried over anhydrous Na₂SO₄, filtered and concentrated under reduced pressure to give a residue. The residue was purified by prep-TLC (SiO₂, dichloromethane:methanol=10:1) to give Compound 49. MS mass calculated for [M+H]⁺(C₂₇H₂₆F₂N₄O₄) requires m/z, 509.2, LCMS found m/z, 509.2; ¹H NMR (400 MHz, CHLOROFORM-d) δ=11.07-10.88 (m, 1H), 7.58 (d, J=9.0 Hz, 2H), 7.49-7.40 (m, 1H), 7.03 (t, J=7.9 Hz, 2H), 6.68 (d, J=8.8 Hz, 2H), 4.40-4.29 (m, 2H), 3.46 (br d, J=6.0 Hz, 2H), 3.37 (br d, J=5.3 Hz, 2H), 3.34-3.26 (m, 1H), 2.16-2.07 (m, 1H), 1.81 (br d, J=4.9 Hz, 2H), 1.77-1.68 (m, 2H), 1.60 (br s, 1H), 1.56-1.48 (m, 1H), 1.28-1.20 (m, 2H), 1.14-1.06 (m, 2H).

### Example 50

### 3-(4-((1R,F,5S)-3-((5-cyclopropyl-3-(2-(trifluoromethoxy)phenyl)isoxazol-4-yl)methoxy)-8-azabicyclo[3.2.1]octan-8-yl)phenyl)-1,2,4-oxadiazol-5(4H)-one

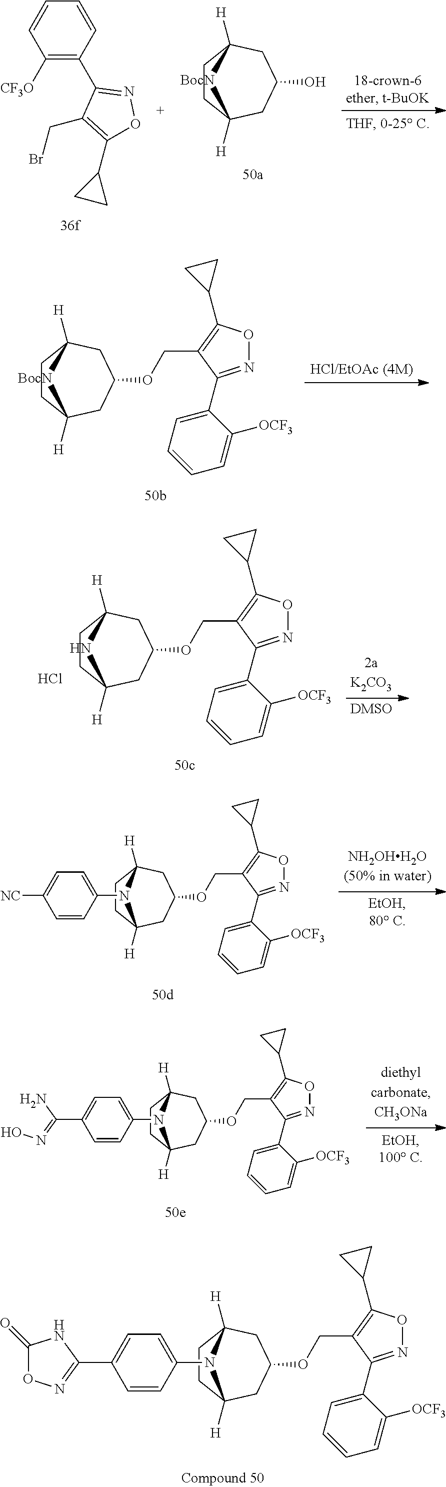

(1R,3R,5S)-tert-butyl 3-((5-cyclopropyl-3-(2-(trifluoromethoxy)phenyl)isoxazol-4-yl)methoxy)-8-azabicyclo[3.2.1]octane-8-carboxylate (50b). To a solution of (1R,3R,5S)-tert-butyl 3-hydroxy-8-azabicyclo[3.2.1]octane-8-carboxylate (50a) (125.53 mg, 552.27 umol) in THF (5 mL) was added 4-(bromomethyl)-5-cyclopropyl-3-(2-(trifluoromethoxy)phenyl)isoxazole (36f) (200 mg, 552.27 umol) and 18-CROWN-6 (218.96 mg, 828.41 umol). The mixture was cooled to 0° C. and added t-BuOK (1 M in THF, 828.41 uL) dropwise at this temperature. The resulting mixture was warmed to 15° C. and stirred for 4 hours. The reaction mixture was diluted with water (10 mL) and extracted with ethyl acetate (15 mL*3). The combined organic phase was washed with brine (10 mL), dried over anhydrous Na₂SO₄, filtered and the filtrate was concentrated under reduced pressure. The residue was purified by column chromatography (SiO₂, petroleum ether:ethyl acetate=10:1 to 5:1) to give 50b. MS mass calculated for [M+H]⁺(C₂₆H₃₁F₃N₂O₅) requires m/z, 509.2, LCMS found m/z, 509.4; ¹H NMR (400 MHz, CHLOROFORM-d) δ=7.62-7.47 (m, 2H), 7.42-7.33 (m, 2H), 4.29 (br d, J=4.9 Hz, 2H), 4.17-3.95 (m, 2H), 3.58-3.51 (m, 1H), 2.16-2.07 (m, 1H), 1.98-1.82 (m, 2H), 1.81-1.71 (m, 4H), 1.66 (br s, 1H), 1.62 (br s, 1H), 1.44 (s, 9H), 1.27-1.18 (m, 2H), 1.15-1.07 (m, 2H).

4-(((1R,3R,5S)-8-azabicyclo[3.2.1]octan-3-yloxy)methyl)-5-cyclopropyl-3-(2-(trifluoromethoxy)phenyl)isoxazole (50c). A solution of (1R,3R,5S)-tert-butyl 3-((5-cyclopropyl-3-(2-(trifluoromethoxy)phenyl)isoxazol-4-yl)methoxy)-8-azabicyclo[3.2.1]octane-8-carboxylate (50b) (180 mg, 353.96 umol) in HCl/ethyl acetate (4 M, 6.00 mL) was stirred at 15° C. for 2 hours. The reaction mixture was concentrated under reduced pressure to give 50c. MS mass calculated for [M+H]⁺(C₂₁H₂₃F₃N₂O₃) requires m/z, 409.2, LCMS found m/z, 409.1

4-((1R,3R,5S)-3-((5-cyclopropyl-3-(2-(trifluoromethoxy)phenyl)isoxazol-4-yl)methoxy)-8-azabicyclo[3.2.1]octan-8-yl)benzonitrile (50d). To a solution of 4-(((1R,3R,5S)-8-azabicyclo[3.2.1]octan-3-yloxy)methyl)-5-cyclopropyl-3-(2-(trifluoromethoxy)phenyl)isoxazole hydrochloride (50c) (150 mg, 337.17 umol, HCl) in DMSO (3 mL) was added K₂CO₃ (279.60 mg, 2.02 mmol) and 4-fluorobenzonitrile (2a) (204.18 mg, 1.69 mmol) in a sealed tube. The resulting mixture was heated to 100° C. and stirred for 26 hours. The mixture was cooled to room temperature and diluted with water (5 mL) and extracted with ethyl acetate (8 mL*3). The combined organic phase was washed with brine (5 mL*3), dried over anhydrous Na₂SO₄, filtered and the filtrate was concentrated under reduced pressure. The residue was purified by column chromatography (SiO₂, petroleum ether:ethyl acetate=10:1 to 5:1) to give 50d. MS mass calculated for [M+H]⁺(C₂₈H₂₆F₃N₃O₃) requires m/z, 510.2, LCMS found m/z, 510.1; ¹H NMR (400 MHz, CHLOROFORM-d) δ=7.60-7.48 (m, 2H), 7.44 (d, J=8.8 Hz, 2H), 7.39 (t, J=7.3 Hz, 2H), 6.64 (d, J=8.8 Hz, 2H), 4.31 (s, 2H), 4.13-4.07 (m, 2H), 3.44 (t, J=4.6 Hz, 1H), 2.15-2.07 (m, 1H), 2.02-1.92 (m, 2H), 1.92-1.82 (m, 4H), 1.60 (br d, J=14.9 Hz, 2H), 1.26-1.20 (m, 2H), 1.16-1.06 (m, 2H).

(Z)-4-((1R,3R,5S)-3-((5-cyclopropyl-3-(2-(trifluoromethoxy)phenyl)isoxazol-4-yl)methoxy)-8-azabicyclo[3.2.1]octan-8-yl)-N′-hydroxybenzimidamide (50e). To a solution of 4-((1R,3R,5S)-3-((5-cyclopropyl-3-(2-(trifluoromethoxy)phenyl)isoxazol-4-yl)methoxy)-8-azabicyclo[3.2.1]octan-8-yl)benzonitrile (50d) (110 mg, 215.89 umol) in ethanol (1 mL) was added hydroxylamine (0.5 mL, 50% in water) and the mixture was stirred for 4 hours at 80° C. The reaction mixture was diluted with water (5 mL) and extracted with dichloromethane (10 mL*2). The combined organic phase was washed with brine (10 mL), dried over anhydrous Na₂SO₄, filtered and the filtrate was concentrated under reduced pressure to give 50e. MS mass calculated for [M+H]⁺(C₂₈H₂₉F₃N₄O₄) requires m/z, 543.2, LCMS found m/z, 543.4; ¹H NMR (400 MHz, CHLOROFORM-d) δ=7.60-7.44 (m, 4H), 7.39 (br t, J=7.0 Hz, 2H), 6.69 (br d, J=8.8 Hz, 2H), 4.78 (br s, 2H), 4.30 (s, 2H), 4.16-4.00 (m, 2H), 3.41 (br d, J=4.4 Hz, 1H), 2.18-2.08 (m, 1H), 2.02-1.83 (m, 6H), 1.54 (br d, J=14.5 Hz, 2H), 1.26-1.17 (m, 2H), 1.16-1.07 (m, 2H).

3-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)-1,2,4-oxadiazol-5(4H)-one (Compound 50). To a solution of (Z)-4-((1R,3R,5S)-3-((5-cyclopropyl-3-(2-(trifluoromethoxy)phenyl)isoxazol-4-yl)methoxy)-8-azabicyclo[3.2.1]octan-8-yl)-N′-hydroxybenzimidamide (50e) (75 mg, 138.24 umol) in ethanol (2 mL) was added diethyl carbonate (979.80 mg, 8.29 mmol, 1.00 mL) and CH₃ONa (56.44 mg, 829.42 umol, 30%) in a sealed tube. The resulting mixture was heated to 100° C. and stirred for 2 hours and was concentrated under reduced pressure. The residue was purified by prep-HPLC (neutral condition; column: Waters Xbridge BEH C18 100*25 mm*5 um; mobile phase: [water (10 mM NH₄HCO₃)-ACN]; B %: 35%-65%, 10 min) to give Compound 50 (30 mg, 52.57 umol, 38.03% yield, 99.626% purity) as a white solid. MS mass calculated for [M+H]⁺(C₂₉H₂₇F₃N₄O₅) requires m/z, 569.2, LCMS found m/z, 569.2; ¹H NMR (400 MHz, CHLOROFORM-d) δ=7.51-7.48 (m, 1H), 7.51-7.48 (m, 1H), 7.62-7.47 (m, 2H), 7.44-7.35 (m, 2H), 6.74 (d, J=9.0 Hz, 2H), 4.38-4.24 (m, 2H), 4.14 (br s, 2H), 3.48-3.41 (m, 1H), 2.12 (tt, J=5.1, 8.5 Hz, 1H), 2.03-1.78 (m, 6H), 1.61 (br d, J=14.6 Hz, 2H), 1.28-1.18 (m, 2H), 1.14-1.07 (m, 2H).

### Example 51

### 5-(4-(4-((5-cyclopropyl-3-(2-(trifluoromethoxy)phenyl)isoxazol-4-yl)methoxy)piperidin-1-yl)phenyl)-1,3,4-oxadiazol-2(3H)-one

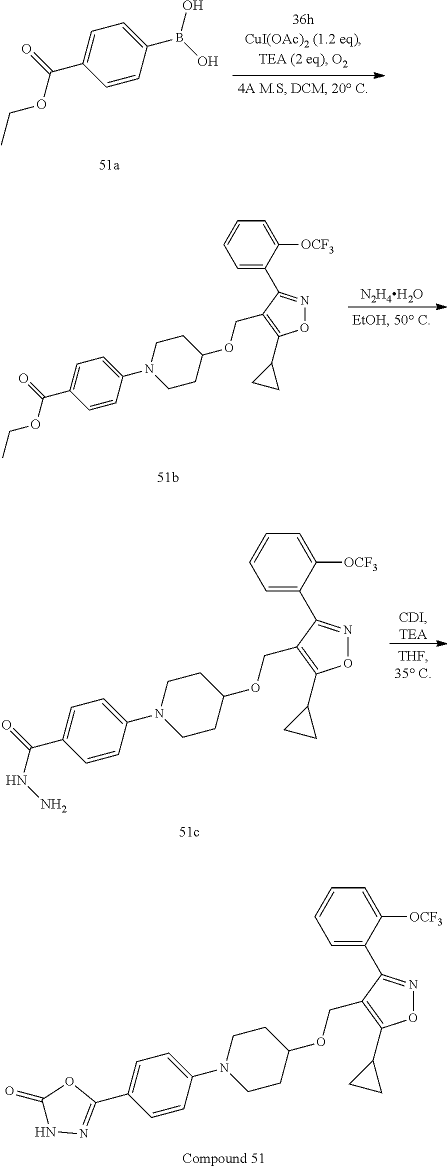 [SMILES: CCOC(=O)c1ccc(B(O)O)cc1 ; CCOC(=O)c1ccc(N2CCC(OCc3c(-c4ccccc4OC(F)(F)F)noc3C3CC3)CC2)cc1 ; "N2H4·H2O" ; NNC(=O)c1ccc(N2CCC(OCc3c(-c4ccccc4OC(F)(F)F)noc3C3CC3)CC2)cc1 ; "CDI,TEA" ; O=c1[nH]nc(-c2ccc(N3CCC(OCc4c(-c5ccccc5OC(F)(F)F)noc4C4CC4)CC3)cc2)o1]

Ethyl 4-(4-((5-cyclopropyl-3-(2-(trifluoromethoxy)phenyl)isoxazol-4-yl)methoxy)piperidin-1-yl)benzoate (51b). To a solution of 5-cyclopropyl-4-((piperidin-4-yloxy)methyl)-3-(2-(trifluoromethoxy)phenyl)isoxazole (36h) (75 mg, 196.14 umol) and (4-(ethoxycarbonyl)phenyl)boronic acid (51a) (76.10 mg, 392.28 umol) in dichloromethane (10 mL) was added Cu(OAc)₂ (42.75 mg, 235.37 umol), 4 A M.S. (20 mg), TEA (39.70 mg, 392.28 umol, 54.60 uL) at 20° C. The suspension was degassed under vacuum and purged with O₂ several times, then stirred under O₂ ballon at 20° C. for 16 hours. The mixture was filtered and the filter cake was washed by dichloromethane (20 mL). The combined filtrate was concentrated under reduced pressure. Water (5 mL) and ethyl acetate (5 mL) were added to the residue and the phases were separated. The aqueous phase was extracted with ethyl acetate (5 mL*4). The combined organic phase was washed with brine (10 mL*2), dried with anhydrous Na₂SO₄, filtered and the filtrate was concentrated under reduced pressure. The residue was purified by prep-TLC (SiO₂, petroleum ether:ethyl acetate=3:1) to give 51b. MS mass calculated for [M+H]⁺(C₂₈H₂₉F₃N₂O₅) requires m/z, 531.2, LCMS found m/z 531.2; ¹H NMR (400 MHz, CHLOROFORM-d) δ=7.90 (d, J=8.9 Hz, 2H), 7.57 (d, J=7.5 Hz, 1H), 7.54-7.47 (m, 1H), 7.41-7.35 (m, 2H), 6.86-6.80 (m, 2H), 4.41 (s, 2H), 4.33 (q, J=7.2 Hz, 2H), 3.53-3.44 (m, 3H), 3.03 (ddd, J=3.3, 9.1, 12.7 Hz, 2H), 2.18-2.10 (m, 1H), 1.87-1.79 (m, 2H), 1.62-1.57 (m, 1H), 1.53 (br d, J=3.9 Hz, 1H), 1.37 (t, J=7.1 Hz, 3H), 1.27-1.22 (m, 2H), 1.14-1.08 (m, 2H).

4-(4-((5-cyclopropyl-3-(2-(trifluoromethoxy)phenyl)isoxazol-4-yl)methoxy)piperidin-1-yl)benzohydrazide (51c). To a solution of ethyl 4-(4-((5-cyclopropyl-3-(2-(trifluoromethoxy)phenyl)isoxazol-4-yl)methoxy)piperidin-1-yl)benzoate (51b) (50 mg, 94.24 umol) in ethanol (5 mL) was added hydrazine hydrate (3.09 g, 61.73 mmol, 3 mL) at 20° C. The reaction was stirred at 50° C. for 16 hours in tube and was concentrated under reduced pressure to remove solvent to give crude 51c. MS mass calculated for [M+H]⁺(C₂₆H₂₇F₃N₄O₄) requires m/z, 517.2, LCMS found m/z 517.2.

5-(4-(4-((5-cyclopropyl-3-(2-(trifluoromethoxy)phenyl)isoxazol-4-yl)methoxy)piperidin-1-yl)phenyl)-1,3,4-oxadiazol-2(3H)-one (Compound 51). To a mixture of 4-(4-((5-cyclopropyl-3-(2-(trifluoromethoxy)phenyl)isoxazol-4-yl)methoxy)piperidin-1-yl)benzohydrazide (51c) (50 mg, 96.80 umol) in THF (10 mL) was added CDI (47.09 mg, 290.41 umol), TEA (39.18 mg, 387.21 umol, 53.90 uL) at 20° C. The reaction was stirred at 35° C. for 8 hours and was concentrated under reduced pressure to remove solvent. Water (5 mL) and ethyl acetate (5 mL) were added to the residue and the phases were separated. The aqueous phase was extracted with ethyl acetate (5 mL*4). The combined organic phase was washed with brine (10 mL*2), dried with anhydrous Na₂SO₄, filtered and the filtrate was concentrated under reduced pressure and the residue was purified by prep-HPLC (neutral condition: column: Waters Xbridge BEH C18 100*25 mm*5 um; mobile phase: [water(10 mM NH₄HCO₃)-ACN]; B %: 40%-70%, 10 min) to give Compound 51. MS mass calculated for [M+H]⁺(C₂₇H₂₅F₃N₄O₅) requires m/z, 543.2, LCMS found m/z 543.2; ¹H NMR (400 MHz, CHLOROFORM-d) δ=8.49 (br s, 1H), 7.69 (d, J=9.0 Hz, 2H), 7.57 (dd, J=1.7, 7.8 Hz, 1H), 7.54-7.47 (m, 1H), 7.42-7.35 (m, 2H), 6.88 (d, J=8.9 Hz, 2H), 4.41 (s, 2H), 3.53-3.43 (m, 3H), 3.04 (ddd, J=3.4, 9.0, 12.7 Hz, 2H), 2.15 (tt, J=5.1, 8.4 Hz, 1H), 1.88-1.78 (m, 2H), 1.63-1.57 (m, 2H), 1.28-1.22 (m, 2H), 1.15-1.08 (m, 2H).

### Example 52

### 5-(4-(4-((5-cyclopropyl-3-(2-(trifluoromethoxy)phenyl)isoxazol-4-yl)methoxy)piperidin-1-yl)phenyl)isoxazol-3(2H)-one

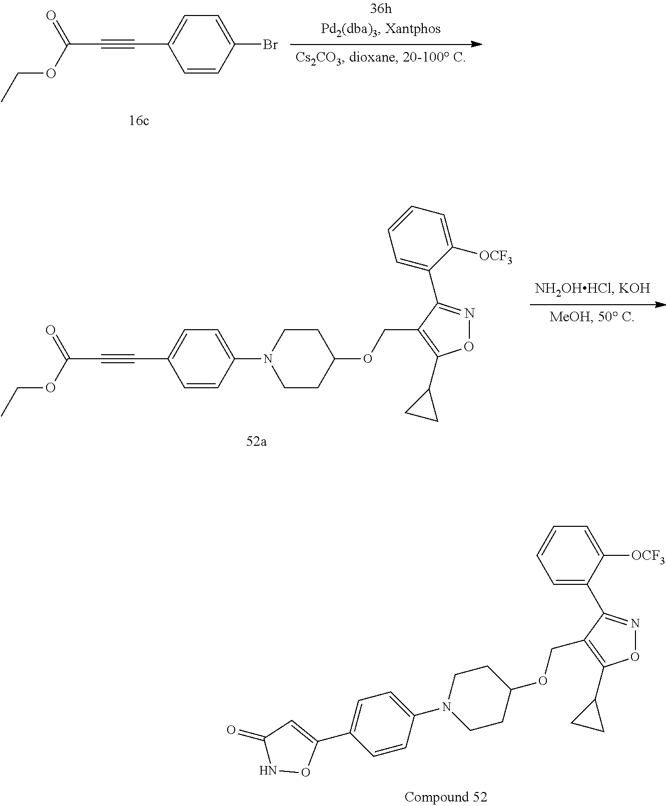 [SMILES: CCOC(=O)C#Cc1ccc(Br)cc1 ; CCOC(=O)C#Cc1ccc(N2CCC(OCc3c(-c4ccccc4OC(F)(F)F)noc3C3CC3)CC2)cc1 ; O=c1cc(-c2ccc(N3CCC(OCc4c(-c5ccccc5OC(F)(F)F)noc4C4CC4)CC3)cc2)o[nH]1]

Ethyl 3-(4-(4-((5-cyclopropyl-3-(2-(trifluoromethoxy)phenyl)isoxazol-4-yl)methoxy)piperidin-1-yl)phenyl)propiolate (52a). To a solution of 5-cyclopropyl-4-((piperidin-4-yloxy)methyl)-3-(2-(trifluoromethoxy)phenyl)isoxazole hydrochloride (36h) (190 mg, 453.64 umol) and ethyl 3-(4-bromophenyl)propiolate (16c) (229.62 mg, 907.27 umol) in 1,4-dioxane (10 mL) was added Cs₂CO₃ (591.22 mg, 1.81 mmol), Xantphos (52.50 mg, 90.73 umol) and Pd₂(dba)₃ (41.54 mg, 45.36 umol) at 20° C. The mixture was degassed and purged with N₂ 3 times and was stirred at 100° C. for 16 hours under N₂ atmosphere. The mixture was filtered and the filter cake was washed by dichloromethane (20 mL). The combined filtrate was concentrated under reduced pressure to remove solvent. Water (5 mL) and ethyl acetate (5 mL) were added into the residue and the phases were separated. The aqueous phase was extracted with ethyl acetate (5 mL*4). The combined organic phase was washed with brine (20 mL*2), dried with anhydrous Na₂SO₄, filtered and the filtrate was concentrated under reduced pressure. The residue was purified by prep-TLC (SiO₂, petroleum ether:ethyl acetate=3:1) to give 52a. MS mass calculated for [M+H]⁺(C₃₀H₂₉F₃N₂O₅) requires m/z, 555.2, LCMS found m/z 555.2; ¹H NMR (400 MHz, CHLOROFORM-d) δ=7.59-7.55 (m, 1H), 7.55-7.48 (m, 1H), 7.46 (d, J=8.9 Hz, 2H), 7.41-7.35 (m, 2H), 6.78 (d, J=8.9 Hz, 2H), 4.40 (s, 2H), 4.29 (q, J=7.1 Hz, 2H), 3.50-3.41 (m, 3H), 3.01 (ddd, J=3.4, 8.9, 12.7 Hz, 2H), 2.18-2.09 (m, 1H), 1.85-1.77 (m, 2H), 1.61-1.52 (m, 2H), 1.35 (t, J=7.2 Hz, 3H), 1.26-1.22 (m, 2H), 1.13-1.08 (m, 2H).

5-(4-(4-((5-cyclopropyl-3-(2-(trifluoromethoxy)phenyl)isoxazol-4-yl)methoxy)piperidin-1-yl)phenyl)isoxazol-3(2H)-one (Compound 52). To a mixture of ethyl 3-(4-(4-((5-cyclopropyl-3-(2-(trifluoromethoxy)phenyl)isoxazol-4-yl)methoxy)piperidin-1-yl)phenyl)propiolate (52a) (90 mg, 162.29 umol) in methanol (6 mL) was added hydroxylamine; hydrochloride (112.78 mg, 1.62 mmol) and KOH (163.91 mg, 2.92 mmol) at 20° C. The mixture was stirred at 50° C. for 16 hours and was concentrated under reduced pressure to remove solvent. Water (5 mL) and ethyl acetate (5 mL) were added into the residue and the phases were separated. The aqueous phase was extracted with ethyl acetate (5 mL*4). The combined organic phase was washed with brine (20 mL*2), dried with anhydrous Na₂SO₄, filtered and concentrated under reduced pressure and purified by prep-HPLC (neutral condition: column: Waters Xbridge BEH C18 100*25 mm*5 um; mobile phase: [water (10 mM NH₄HCO₃)-ACN]; B %: 25%-55%, 10 min) to give Compound 52. MS mass calculated for [M+H]⁺(C₂₈H₂₆F₃N₃O₅) requires m/z, 542.2, LCMS found m/z 542.2; ¹H NMR (400 MHz, CHLOROFORM-d) δ=7.58 (br d, J=7.9 Hz, 3H), 7.51 (br t, J=7.8 Hz, 1H), 7.41-7.35 (m, 2H), 6.88 (br d, J=8.6 Hz, 2H), 5.99 (s, 1H), 4.41 (s, 2H), 3.46 (br d, J=4.2 Hz, 3H), 2.99 (br t, J=9.3 Hz, 2H), 2.19-2.11 (m, 1H), 1.83 (br s, 1H), 1.89-1.79 (m, 1H), 1.59 (br d, J=8.4 Hz, 2H), 1.27-1.22 (m, 2H), 1.14-1.08 (m, 2H).

### Example 53

### 3-(4-(4-((5-cyclopropyl-3-(2,6-dichlorophenyl)isoxazol-4-yl)methoxy)-3,3-difluoropiperidin-1-yl)phenyl)-1,2,4-oxadiazol-5(4H)-one

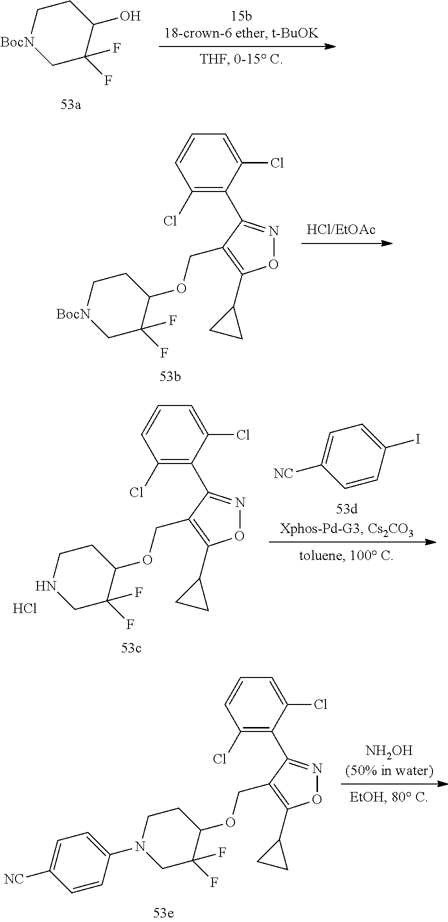

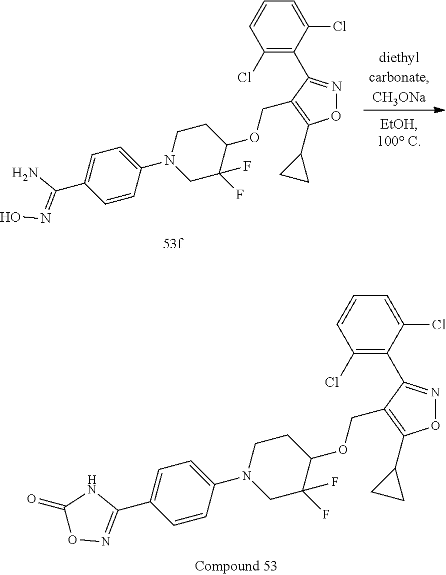 [SMILES: N/C(=N\O)c1ccc(N2CCC(OCc3c(-c4c(Cl)cccc4Cl)noc3C3CC3)C(F)(F)C2)cc1 ; "diethyl carbonate,CH3ONa" ; O=c1[nH]c(-c2ccc(N3CCC(OCc4c(-c5c(Cl)cccc5Cl)noc4C4CC4)C(F)(F)C3)cc2)no1]

Tert-butyl 4-((5-cyclopropyl-3-(2,6-dichlorophenyl)isoxazol-4-yl)methoxy)-3,3-difluoropiperidine-1-carboxylate (53b). To a solution of 4-(bromomethyl)-5-cyclopropyl-3-(2,6-dichlorophenyl)isoxazole (15b) (200 mg, 576.31 umol) in THF (5 mL) was added tert-butyl 3,3-difluoro-4-hydroxypiperidine-1-carboxylate (53a) (136.73 mg, 576.31 umol) and 18-CROWN-6 (228.49 mg, 864.47 umol). The mixture was cooled to 0° C. and t-BuOK (1 M, 864.47 uL) was added dropwise. After the addition, the reaction mixture was warmed to 15° C. and stirred for 4 hours and was diluted with water (10 mL) and extracted with ethyl acetate (15 mL*3). The combined organic phase was washed with brine (10 mL), dried over anhydrous Na₂SO₄, filtered and the filtrate was concentrated under reduced pressure. The residue was purified by column chromatography (SiO₂, petroleum ether:ethyl acetate=10:1 to 5:1) to give 53b. MS mass calculated for [M+H]⁺(C₂₃H₂₆Cl₂F₂N₂O₄) requires m/z, 503.1/505.1, LCMS found m/z, 503.1/505.1; ¹H NMR (400 MHz, CHLOROFORM-d) δ=7.47-7.40 (m, 2H), 7.38-7.32 (m, 1H), 4.58 (br d, J=10.8 Hz, 1H), 4.38 (d, J=12.2 Hz, 1H), 3.76 (br s, 1H), 3.63-3.46 (m, 2H), 3.32 (br s, 1H), 3.03 (br s, 1H), 2.19-2.08 (m, 1H), 1.68 (br d, J=9.8 Hz, 1H), 1.56 (m, 1H), 1.49-1.39 (m, 9H), 1.30-1.24 (m, 2H), 1.19-1.11 (m, 2H).

5-cyclopropyl-3-(2,6-dichlorophenyl)-4-(((3,3-difluoropiperidin-4-yl)oxy)methyl)isoxazole hydrochloride (53c). A solution of tert-butyl 4-((5-cyclopropyl-3-(2,6-dichlorophenyl)isoxazol-4-yl)methoxy)-3,3-difluoropiperidine-1-carboxylate (53b) (190 mg, 377.46 umol) in HCl/ethyl acetate (5 mL, 4 M) was stirred for 2 hours at 15° C. The reaction mixture was concentrated under reduced pressure to give 53c. MS mass calculated for [M+H]⁺(C₁₈H₁₈Cl₂F₂N₂O₂) requires m/z, 403.1/405.1, LCMS found m/z, 403.0/405.0.

4-(4-((5-cyclopropyl-3-(2,6-dichlorophenyl)isoxazol-4-yl)methoxy)-3,3-difluoropiperidin-1-yl)benzonitrile (53e). To a solution of 5-cyclopropyl-3-(2,6-dichlorophenyl)-4-(((3,3-difluoropiperidin-4-yl)oxy)methyl)isoxazole hydrochloride (53c) (100 mg, 247.99 umol) and 4-iodobenzonitrile (53d) (85.19 mg, 371.98 umol) in toluene (2 mL) was added Xphos-Pd-G3 (20.99 mg, 24.80 umol) and Cs₂CO₃ (161.60 mg, 495.97 umol) under N₂. The suspension was degassed and purged with N₂ 3 times and was heated to 100° C. and stirred for 18 hours. The reaction mixture was cooled to 45° C. and diluted with ethyl acetate (10 mL), 3-mercaptopropyl-functionalized silica gel (100 mg) was added. The mixture was stirred for 2 hours and then filtered. The filter cake was rinsed with ethyl acetate (10 mL) and the combined filtrate was concentrated under reduced pressure. The residue was purified by prep-TLC (SiO₂, petroleum ether:ethyl acetate=3:1) to give 53e. MS mass calculated for [M+H]⁺(C₂₅H₂₁Cl₂F₂N₃O₂) requires m/z, 504.1/506.1 LCMS found m/z, 504.3/506.3; ¹H NMR (400 MHz, CHLOROFORM-d) δ=7.53-7.46 (m, 2H), 7.44-7.37 (m, 2H), 7.44-7.37 (m, 1H), 6.82 (d, J=8.8 Hz, 2H), 4.63 (d, J=11.7 Hz, 1H), 4.42 (d, J=11.9 Hz, 1H), 3.68-3.51 (m, 2H), 3.34 (dq, J=2.8, 13.2 Hz, 2H), 3.21-3.04 (m, 1H), 2.14 (tt, J=5.0, 8.4 Hz, 1H), 1.92-1.79 (m, 1H), 1.76-1.63 (m, 1H), 1.34-1.22 (m, 2H), 1.21-1.08 (m, 2H).

(Z)-4-(4-((5-cyclopropyl-3-(2,6-dichlorophenyl)isoxazol-4-yl)methoxy)-3,3-difluoropiperidin-1-yl)-N′-hydroxybenzimidamide (53f). To a solution of 4-(4-((5-cyclopropyl-3-(2,6-dichlorophenyl)isoxazol-4-yl)methoxy)-3,3-difluoropiperidin-1-yl)benzonitrile (53e) (40 mg, 79.31 umol) in ethanol (1 mL) was added hydroxylamine (0.5 mL, 50% solution) in a sealed tube at 15°. The mixture was heated to 80° C. and stirred for 4 hours and was diluted with water (5 mL) and extracted with ethyl acetate (10 mL*2). The combined organic phase was washed with brine (10 mL), dried over anhydrous Na₂SO₄, filtered and the filtrate was concentrated to give 53e. MS mass calculated for [M+H]⁺(C₂₅H₂₄Cl₂F₂N₄O₃) requires m/z, 537.1/539.1, LCMS found m/z, 537.0/538.9; ¹H NMR (400 MHz, CHLOROFORM-d) δ=7.51 (d, J=8.8 Hz, 2H), 7.44-7.36 (m, 2H), 7.36-7.28 (m, 1H), 6.85 (d, J=9.0 Hz, 2H), 4.79 (br s, 2H), 4.63 (d, J=11.9 Hz, 1H), 4.42 (d, J=11.9 Hz, 1H), 3.60 (br s, 1H), 3.48-3.37 (m, 1H), 3.30 (br d, J=13.0 Hz, 1H), 3.22 (br s, 1H), 3.06 (br t, J=10.3 Hz, 1H), 2.22-2.08 (m, 1H), 1.88 (br s, 1H), 1.74 (br s, 1H), 1.35-1.21 (m, 2H), 1.15 (br dd, J=2.6, 8.2 Hz, 2H).

### 3-(4-(4-((5-cyclopropyl-3-(2,6-dichlorophenyl)isoxazol-4-yl)methoxy)-3,3-difluoropiperidin-1-yl)phenyl)-1,2,4-oxadiazol-5(4H)-one (Compound 53)

To a solution of (Z)-4-(4-((5-cyclopropyl-3-(2,6-dichlorophenyl)isoxazol-4-yl)methoxy)-3,3-difluoropiperidin-1-yl)-N′-hydroxybenzimidamide (53f) (40 mg, 74.43 umol) in ethanol (0.3 mL) was added diethyl carbonate (527.58 mg, 4.47 mmol, 541.11 uL) and CH₃ONa (101.31 mg, 446.61 umol, 30% solution) in a sealed tube. The mixture was heated to 100° C. and stirred for 2 hours and was diluted with water (10 mL), extracted with ethyl acetate (10 mL*2). The combined organic phase was washed with brine (5 mL), dried over anhydrous Na₂SO₄, filtered and the filtrate was concentrated under reduced pressure. The residue was purified by prep-HPLC (neutral condition; column: Waters Xbridge BEH C18 100*25 mm*5 um; mobile phase: [water (10 mM NH₄HCO₃)-ACN]; B %: 30%-60%, 10 min) to give Compound 53. MS mass calculated for [M+H]⁺(C₂₆H₂₂Cl₂F₂N₄O₄) requires m/z, 563.1/565.1, LCMS found m/z, 563.1/565.2; ¹H NMR (400 MHz, CHLOROFORM-d) δ=7.63 (br d, J=8.8 Hz, 2H), 7.48-7.37 (m, 2H), 7.37-7.30 (m, 1H), 6.91 (br d, J=8.8 Hz, 2H), 4.64 (d, J=11.7 Hz, 1H), 4.43 (d, J=12.2 Hz, 1H), 3.68-3.52 (m, 2H), 3.44-3.27 (m, 2H), 3.14 (br t, J=10.5 Hz, 1H), 2.21-2.09 (m, 1H), 1.88 (br s, 1H), 1.76-1.65 (m, 1H), 1.37-1.22 (m, 2H), 1.22-1.08 (m, 2H).

### Example 54

### 3-(4-(4-((5-cyclopropyl-3-(2-(trifluoromethoxy)phenyl)isoxazol-4-yl)methoxy)-3,3-difluoropiperidin-1-yl)phenyl)-1,2,4-oxadiazol-5(4H)-one

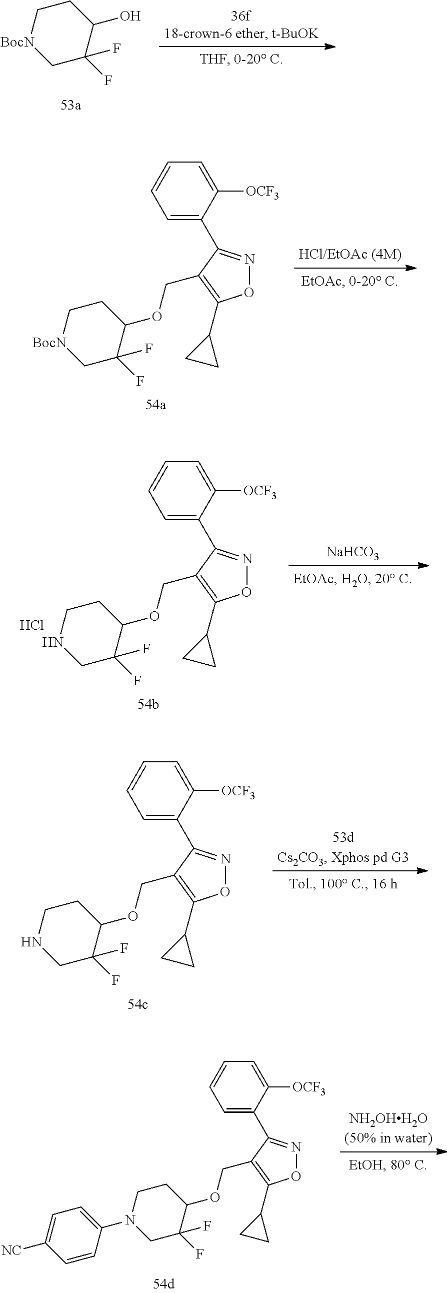

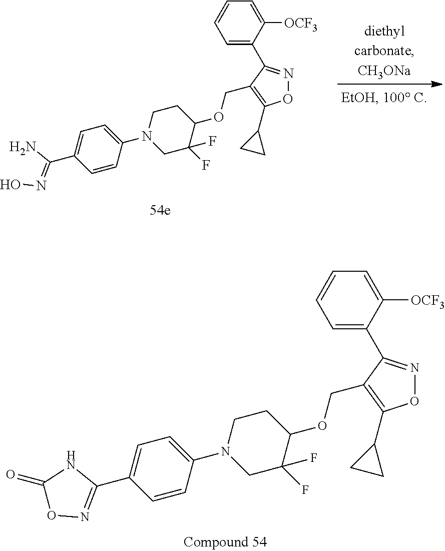 [SMILES: N/C(=N\O)c1ccc(N2CCC(OCc3c(-c4ccccc4OC(F)(F)F)noc3C3CC3)C(F)(F)C2)cc1 ; "diethyl carbonate,CH3ONa" ; O=c1[nH]c(-c2ccc(N3CCC(OCc4c(-c5ccccc5OC(F)(F)F)noc4C4CC4)C(F)(F)C3)cc2)no1]

Tert-butyl 4-((5-cyclopropyl-3-(2-(trifluoromethoxy)phenyl)isoxazol-4-yl)methoxy)-3,3-difluoropiperidine-1-carboxylate (54a). To a solution of tert-butyl 3,3-difluoro-4-hydroxypiperidine-1-carboxylate (53a) (255.49 mg, 1.08 mmol) in THF (20 mL) was added 18-crown-6 (328.45 mg, 1.24 mmol), t-BuOK (1 M, 1.24 mL) at 0° C. The reaction was degassed and purged with N₂ 3 times. The mixture was stirred at 20° C. for 0.5 hour, then 4-(bromomethyl)-5-cyclopropyl-3-(2-(trifluoromethoxy)phenyl)isoxazole (36f) (300 mg, 828.41 umol) was added and the mixture was stirred at 20° C. for 1.5 hours. The reaction mixture was concentrated under reduced pressure to remove solvent. Water (35 mL) and ethyl acetate (55 mL) were added into the reaction mixture and the phases were separated. The aqueous phase was extracted with ethyl acetate (25 mL*4). The combined organic phase was washed with brine (40 mL*2), dried with anhydrous Na₂SO₄, filtered and the filtrate was concentrated under reduced pressure. The residue was purified by flash silica gel chromatography (ISCO®; 20 g Sepa Flash® Silica Flash Column, Eluent of 0-20% ethyl acetate/Petroleum ether gradient @ 150 mL/min) to give 54a. MS mass calculated for [M+H]⁺(C₂₄H₂₇F₅N₂O₅) requires m/z, 519.2, LCMS found m/z, 519.2; ¹H NMR (400 MHz, CHLOROFORM-d) δ=7.57-7.51 (m, 1H), 7.57-7.51 (m, 1H), 7.40 (t, J=7.2 Hz, 2H), 4.63 (br d, J=11.2 Hz, 1H), 4.45 (d, J=11.8 Hz, 1H), 3.75 (br s, 1H), 3.59-3.52 (m, 2H), 3.50-3.30 (m, 2H), 3.05 (br s, 1H), 2.15-2.08 (m, 1H), 1.71 (br s, 1H), 1.45 (s, 9H), 1.26-1.22 (m, 2H), 1.16-1.10 (m, 2H).

5-cyclopropyl-4-(((3,3-difluoropiperidin-4-yl)oxy)methyl)-3-(2-(trifluoromethoxy)phenyl)isoxazole (54b). To a solution of tert-butyl 4-((5-cyclopropyl-3-(2-(trifluoromethoxy)phenyl)isoxazol-4-yl)methoxy)-3,3-difluoropiperidine-1-carboxylate (54a) (420 mg, 810.07 umol) in ethyl acetate (2 mL) was added HCl/ethyl acetate 16 mL, 4M) at 20° C. for 2 hours. The reaction mixture was concentrated under reduced pressure to give 54b. MS mass calculated for [M+H]⁺(C₁₉H₁₉F₅N₂O₃) requires m/z, 419.1, LCMS found m/z, 419.1.

5-cyclopropyl-4-(((3,3-difluoropiperidin-4-yl)oxy)methyl)-3-(2-(trifluoromethoxy)phenyl)isoxazole (54c). To a solution of 5-cyclopropyl-4-(((3,3-difluoropiperidin-4-yl)oxy)methyl)-3-(2-(trifluoromethoxy)phenyl)isoxazole hydrochloride (54b) (300 mg, 659.60 umol) in ethyl acetate (12 mL) and H₂O (1.5 mL) was added NaHCO₃(443.31 mg, 5.28 mmol, 205.23 uL) at 20° C. The mixture was stirred at 20° C. for 4 hours. The reaction mixture was separated and the organic phase was dried over anhydrous Na₂SO₄, filtered and the filtrate was concentrated under reduced pressure to give 54c. MS mass calculated for [M+H]⁺(C₁₉H₁₉F₅N₂O₃) requires m/z, 419.1, LCMS found m/z, 419.1.

4-(4-((5-cyclopropyl-3-(2-(trifluoromethoxy)phenyl)isoxazol-4-yl)methoxy)-3,3-difluoropiperidin-1-yl)benzonitrile (54d). To a solution of 5-cyclopropyl-4-(((3,3-difluoropiperidin-4-yl)oxy)methyl)-3-(2-(trifluoromethoxy)phenyl)isoxazole (54c) (100 mg, 239.03 umol) and 4-iodobenzonitrile (53d) (82.11 mg, 358.55 umol) in toluene (10 mL) was added Cs₂CO₃ (155.76 mg, 478.06 umol) and Xphos-Pd-G3 (20.23 mg, 23.90 umol) at 20° C. The suspension was degassed under vacuum and purged with N₂ several times and was stirred under N₂ at 100° C. for 16 hours. The mixture was concentrated under reduced pressure to remove solvent. Water (5 mL) and ethyl acetate (5 mL) were added into the residue and the phases were separated. The aqueous phase was extracted with ethyl acetate (5 mL*4). The combined organic phase was washed with brine (20 mL*2), dried with anhydrous Na₂SO₄, filtered and the filtrate was concentrated under reduced pressure. The residue was purified by column chromatography (SiO₂, petroleum ether:ethyl acetate=1:0 to 0:1) to give 54d. MS mass calculated for [M+H]⁺(C₂₆H₂₂F₅N₃O) requires m/z, 520.2, LCMS found m/z 520.2; ¹H NMR (400 MHz, CHLOROFORM-d) δ=7.58-7.46 (m, 4H), 7.42-7.37 (m, 2H), 6.82 (d, J=9.0 Hz, 2H), 4.69 (d, J=11.7 Hz, 1H), 4.50 (d, J=11.7 Hz, 1H), 3.68-3.54 (m, 2H), 3.48-3.28 (m, 2H), 3.19-3.09 (m, 1H), 2.12 (tt, J=5.1, 8.4 Hz, 1H), 1.86 (ddd, J=3.8, 9.9, 13.7 Hz, 1H), 1.77-1.64 (m, 1H), 1.28-1.25 (m, 2H), 1.17-1.12 (m, 2H).

(Z)-4-(4-((5-cyclopropyl-3-(2-(trifluoromethoxy)phenyl)isoxazol-4-yl)methoxy)-3,3-difluoropiperidin-1-yl)-N′-hydroxybenzimidamide (54e). To a solution of 4-(4-((5-cyclopropyl-3-(2-(trifluoromethoxy)phenyl)isoxazol-4-yl)methoxy)-3,3-difluoropiperidin-1-yl)benzonitrile (54d) (60 mg, 115.50 umol) in ethanol (6 mL) was added hydroxylamine (22.89 mg, 346.51 umol, 3 mL, 50% in water) at 20° C. The reaction was degassed and purged with N₂ 3 times and was stirred at 80° C. for 2 hours under N₂ atmosphere. The reaction mixture was concentrated under reduced pressure to remove solvent. The residue was diluted with water (5 mL) and ethyl acetate (5 mL) and the phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, dichloromethane:Methanol=10:1) to give 54e. MS mass calculated for [M+H]⁺(C₂₆H₂₅F₅N₄O₄) requires m/z, 553.2, LCMS found m/z 553.2; ¹H NMR (400 MHz, CHLOROFORM-d) δ=7.59-7.49 (m, 4H), 7.42-7.36 (m, 2H), 6.85 (d, J=8.9 Hz, 2H), 4.79 (br s, 2H), 4.69 (d, J=11.7 Hz, 1H), 4.49 (d, J=11.7 Hz, 1H), 3.65-3.57 (m, 1H), 3.49-3.30 (m, 2H), 3.22-3.14 (m, 1H), 3.13-3.04 (m, 1H), 2.18-2.09 (m, 1H), 1.94-1.83 (m, 1H), 1.72 (br dd, J=3.9, 9.8 Hz, 1H), 1.27-1.22 (m, 2H), 1.16-1.10 (m, 2H)

3-(4-(4-((5-cyclopropyl-3-(2-(trifluoromethoxy)phenyl)isoxazol-4-yl)methoxy)-3,3-difluoropiperidin-1-yl)phenyl)-1,2,4-oxadiazol-5(4H)-one (Compound 54). To a mixture of (Z)-4-(4-((5-cyclopropyl-3-(2-(trifluoromethoxy)phenyl)isoxazol-4-yl)methoxy)-3,3-difluoropiperidin-1-yl)-N′-hydroxybenzimidamide (54e) (60 mg, 108.60 umol) in ethanol (6 mL) was added diethyl carbonate (1.95 g, 16.51 mmol, 2 mL) and CH₃ONa (195.55 mg, 1.09 mmol, 0.6 mL, 30% in MeOH) at 20° C. The reaction was degassed and purged with N₂ 3 times and was stirred at 100° C. for 2 hours. The reaction mixture was concentrated under reduced pressure to remove solvent. The residue was diluted with water (5 mL) and ethyl acetate (5 mL) then the phases were separated. The aqueous phase was extracted with ethyl acetate (5 mL*4). The combined organic phase was washed with brine (20 mL*2), dried with anhydrous Na₂SO₄, filtered and the filtrate was concentrated under reduced pressure. The residue was purified by prep-HPLC (neutral condition: column: Waters Xbridge BEH C18 100*25 mm*5 um; mobile phase: [water(10 mM NH₄HCO₃)-ACN]; B %: 25%-60%, 10 min) to give Compound 54. MS mass calculated for [M+H]⁺(C₂₇H₂₃F₅N₄O₅) requires m/z, 579.2, LCMS found m/z 579.3; ¹H NMR (400 MHz, CHLOROFORM-d) δ=7.63 (d, J=8.9 Hz, 2H), 7.59-7.50 (m, 2H), 7.43-7.37 (m, 2H), 6.92 (d, J=9.0 Hz, 2H), 4.70 (d, J=11.7 Hz, 1H), 4.50 (d, J=11.7 Hz, 1H), 3.68-3.54 (m, 2H), 3.50-3.38 (m, 1H), 3.33 (br d, J=13.3 Hz, 1H), 3.20-3.11 (m, 1H), 2.13 (tt, J=5.0, 8.4 Hz, 1H), 1.89 (ddd, J=3.7, 9.7, 13.5 Hz, 1H), 1.72 (br dd, J=4.3, 9.6 Hz, 1H), 1.28-1.23 (m, 2H), 1.17-1.11 (m, 2H).

### Example 55

### 3-(4-(4-((5-cyclopropyl-3-(2,6-difluorophenyl)isoxazol-4-yl)methoxy)-3,3-difluoropiperidin-1-yl)phenyl)-1,2,4-oxadiazol-5(4H)-one

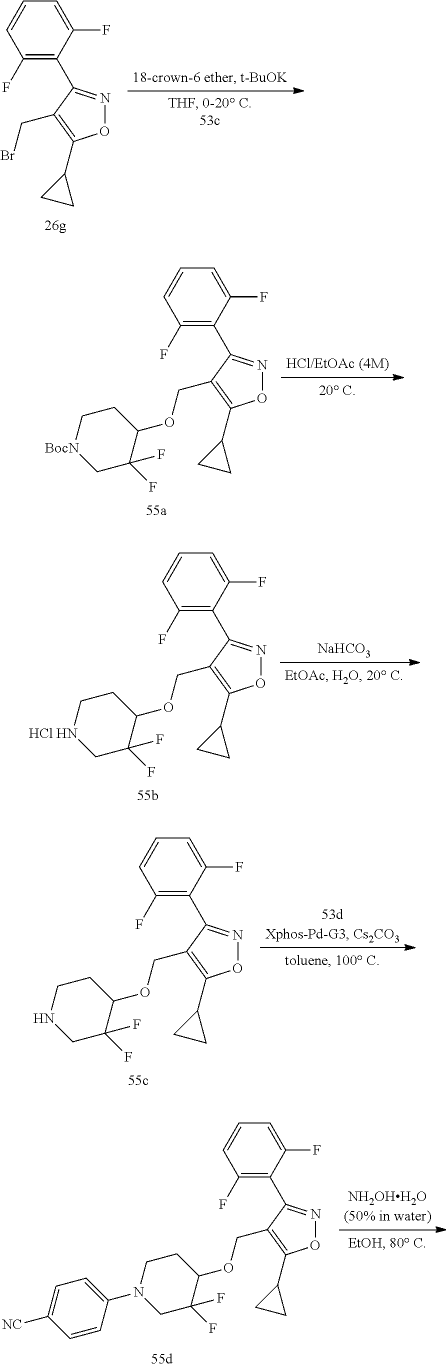

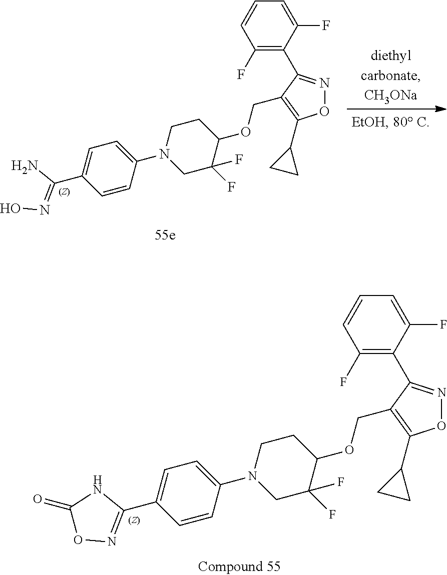 [SMILES: N/C(=N\O)c1ccc(N2CCC(OCc3c(-c4c(F)cccc4F)noc3C3CC3)C(F)(F)C2)cc1 ; "diethyl carbonate,CH3ONa" ; O=c1[nH]c(-c2ccc(N3CCC(OCc4c(-c5c(F)cccc5F)noc4C4CC4)C(F)(F)C3)cc2)no1]

Tert-butyl 4-((5-cyclopropyl-3-(2,6-difluorophenyl)isoxazol-4-yl)methoxy)-3,3-difluoropiperidine-1-carboxylate (55a). To a solution of 4-(bromomethyl)-5-cyclopropyl-3-(2,6-difluorophenyl)isoxazole (26g) (219.02 mg, 923.20 umol) and tert-butyl 3,3-difluoro-4-hydroxypiperidine-1-carboxylate (53c) (290 mg, 923.20 umol) in THF (10 mL) was added 18-CROWN-6 (366.03 mg, 1.38 mmol) and K-BuOK (1 M in THF, 1.38 mL) dropwise at 0° C. The mixture was stirred at 20° C. for 2 hours and was poured into H₂O (10 mL) and extracted with ethyl acetate (20 mL*2). The combined organic layer was washed with brine (10 mL), dried over anhydrous Na₂SO₄, filtered and the filtrate was concentrated under reduced pressure to give a residue. The residue was purified by column chromatography to give 55a. MS mass calculated for [M+H]⁺(C₂₃H₂₆F₄N₂O₄) requires m/z, 471.2, LCMS found m/z, 415.3; ¹H NMR (400 MHz, CHLOROFORM-d) δ=7.44 (tt, J=6.4, 8.5 Hz, 1H), 7.07-6.98 (m, 2H), 4.63 (br d, J=12.0 Hz, 1H), 4.45 (d, J=12.0 Hz, 1H), 3.79 (br d, J=14.2 Hz, 1H), 3.64-3.45 (m, 2H), 3.44-3.24 (m, 1H), 3.05 (br s, 1H), 2.12 (tt, J=5.1, 8.4 Hz, 1H), 1.77-1.64 (m, 1H), 1.56-1.49 (m, 1H), 1.45 (s, 9H), 1.28-1.22 (m, 2H), 1.17-1.10 (m, 2H).

5-cyclopropyl-3-(2,6-difluorophenyl)-4-(((3,3-difluoropiperidin-4-yl)oxy)methyl)isoxazole (55b). To a solution of tert-butyl 4-((5-cyclopropyl-3-(2,6-difluorophenyl)isoxazol-4-yl)methoxy)-3,3-difluoropiperidine-1-carboxylate (55a) (230 mg, 488.89 umol) in ethyl acetate (5 mL) was added HCl/ethyl acetate (5.00 mL, 4 M) at 20° C. and the mixture was stirred at 20° C. for 2 hours. The reaction mixture was concentrated under reduced pressure to give 55b. MS mass calculated for [M+H]⁺(C₁₈H₁₅F₄N₂O₂) requires m/z, 371.1, LCMS found m/z, 371.1.

5-cyclopropyl-3-(2,6-difluorophenyl)-4-(((3,3-difluoropiperidin-4-yl)oxy)methyl)isoxazole (55c). To a solution of 5-cyclopropyl-3-(2,6-difluorophenyl)-4-(((3,3-difluoropiperidin-4-yl)oxy)methyl)isoxazole hydrochloride (55b) (240 mg, 589.97 umol) in ethyl acetate (5 mL) and H₂O (1 mL) was added NaHCO₃(247.82 mg, 2.95 mmol, 114.73 uL) at 20° C. and the mixture was stirred at 20° C. for 2 hours. The reaction mixture was dried over anhydrous Na₂SO₄, filtered and the filtrate was concentrated under reduced pressure to give 55c.

4-(4-((5-cyclopropyl-3-(2,6-difluorophenyl)isoxazol-4-yl)methoxy)-3,3-difluoropiperidin-1-yl)benzonitrile (55d). To a solution of 5-cyclopropyl-3-(2,6-difluorophenyl)-4-(((3,3-difluoropiperidin-4-yl)oxy)methyl)isoxazole (55c) (80 mg, 216.02 umol) and 4-iodobenzonitrile (53d) (74.21 mg, 324.03 umol) in toluene (4 mL) was added[2-(2-aminophenyl)phenyl]palladium(1+); dicyclohexyl-[2-(2,4,6-triisopropylphenyl)phenyl]phosphane; methanesulfonate (18.28 mg, 21.60 umol) and Cs₂CO₃ (140.77 mg, 432.03 umol) at 20° C. The mixture was heated to 100° C. for 16 hours and was poured into H₂O (10 mL) and extracted with ethyl acetate (15 mL*3). The combined organic layer was washed with brine (10 mL), dried over anhydrous Na₂SO₄, filtered and the filtrate was concentrated under reduced pressure to give a residue. The residue was purified by prep-TLC to give 55d. MS mass calculated for [M+H]⁺(C₂₅H₂₁F₄N₃O₂) requires m/z, 472.2, LCMS found m/z, 472.2.

(Z)-4-(4-((5-cyclopropyl-3-(2,6-difluorophenyl)isoxazol-4-yl)methoxy)-3,3-difluoropiperidin-1-yl)-N′-hydroxybenzimidamide (55e). To a solution of 4-(4-((5-cyclopropyl-3-(2,6-difluorophenyl)isoxazol-4-yl)methoxy)-3,3-difluoropiperidin-1-yl)benzonitrile (55d) (50 mg, 106.06 umol) in ethanol (5 mL) was added hydroxylamine (7.01 mg, 106.06 umol, 0.5 mL, 50% in water) at 20° C. and the mixture was heated to 80° C. for 1 hour. Water (10 mL) was added to the reaction mixture and the mixture was extracted by ethyl acetate (10 mL*3). The combined organic phase was washed with brine (20 mL) and dried with anhydrous Na₂SO₄, filtered and the filtrate was concentrated under reduced pressure. The residue was purified by prep-TLC to give 55e. MS mass calculated for [M+H]⁺(C₂₅H₂₄F₄N₄O₃) requires m/z, 505.2, LCMS found m/z, 505.2.

3-(4-(4-((5-cyclopropyl-3-(2,6-difluorophenyl)isoxazol-4-yl)methoxy)-3,3-difluoropiperidin-1-yl)phenyl)-1,2,4-oxadiazol-5(4H)-one (Compound 55). To a solution of (Z)-4-(4-((5-cyclopropyl-3-(2,6-difluorophenyl)isoxazol-4-yl)methoxy)-3,3-difluoropiperidin-1-yl)-N′-hydroxybenzimidamide (55e) (40 mg, 79.29 umol) in ethanol (4 mL) was added diethyl carbonate (468.33 mg, 3.96 mmol, 480.34 uL) and CH₃ONa (71.39 mg, 396.45 umol, 30% in MeOH) at 20° C. in a sealed tube. The mixture was heated to 100° C. for 0.5 hour and water (10 mL) was added to the mixture. The mixture was extracted with ethyl acetate (10 mL*2) and the combined organic phase was washed with brine (20 mL), dried with anhydrous Na₂SO₄, filtered and the filtrate was concentrated under reduced pressure. The residue was purified by prep-TLC to give Compound 55. MS mass calculated for [M+H]⁺(C₂₆H₂₂F₄N₄O₄) requires m/z, 531.2, LCMS found m/z, 531.3; ¹H NMR (400 MHz, CHLOROFORM-d) δ=7.62 (d, J=8.8 Hz, 2H), 7.49-7.39 (m, 1H), 7.03 (t, J=7.8 Hz, 2H), 6.92 (br d, J=8.9 Hz, 2H), 4.69 (d, J=12.0 Hz, 1H), 4.50 (d, J=12.0 Hz, 1H), 3.68-3.53 (m, 2H), 3.48-3.30 (m, 2H), 3.21-3.11 (m, 1H), 2.19-2.09 (m, 1H), 1.87 (br d, J=9.8 Hz, 1H), 1.71 (br dd, J=4.8, 9.9 Hz, 1H), 1.29-1.26 (m, 2H), 1.18-1.12 (m, 2H).

### Example 56

### 3-(5-(4-((5-cyclopropyl-3-(2,6-dichlorophenyl)isoxazol-4-yl)methoxy)piperidin-1-yl)thiazol-2-yl)-1,2,4-oxadiazol-5(4H)-one

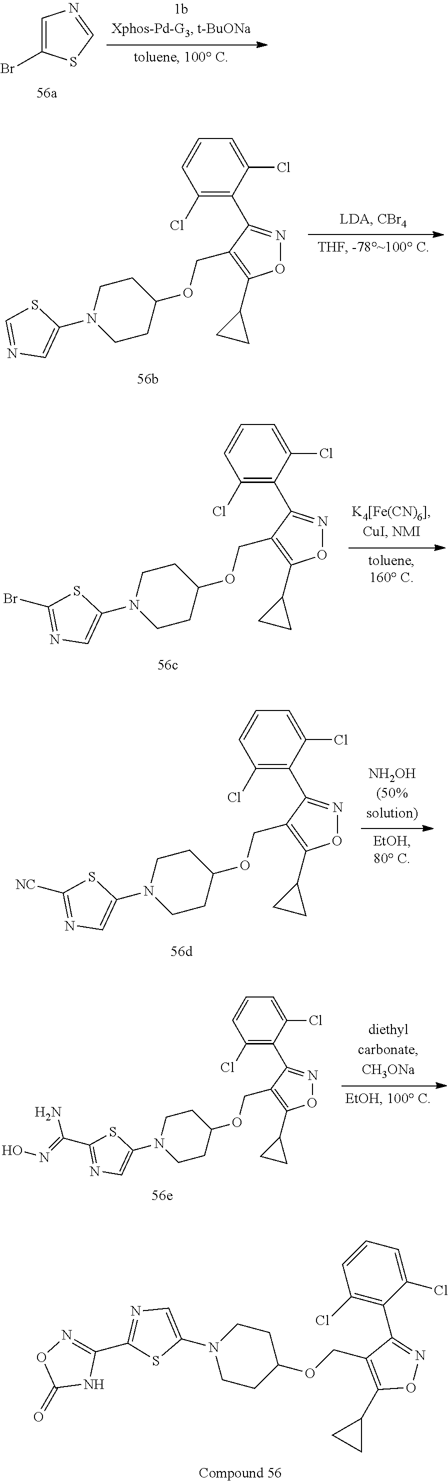 [SMILES: Brc1cncs1 ; Clc1cccc(Cl)c1-c1noc(C2CC2)c1COC1CCN(c2cncs2)CC1 ; Clc1cccc(Cl)c1-c1noc(C2CC2)c1COC1CCN(c2cnc(Br)s2)CC1 ; N#Cc1ncc(N2CCC(OCc3c(-c4c(Cl)cccc4Cl)noc3C3CC3)CC2)s1 ; N/C(=N\O)c1ncc(N2CCC(OCc3c(-c4c(Cl)cccc4Cl)noc3C3CC3)CC2)s1 ; O=c1[nH]c(-c2ncc(N3CCC(OCc4c(-c5c(Cl)cccc5Cl)noc4C4CC4)CC3)s2)no1]

5-cyclopropyl-3-(2,6-dichlorophenyl)-4-(((1-(thiazol-5-yl)piperidin-4-yl)oxy)methyl)isoxazole (56b). To a solution of 5-cyclopropyl-3-(2,6-dichlorophenyl)-4-((piperidin-4-yloxy)methyl)isoxazole hydrochloride (1b) (1 g, 2.48 mmol) and 5-bromothiazole (56a) (487.52 mg, 2.97 mmol) in toluene (10 mL) was added Xphos-Pd-G3 (419.31 mg, 495.38 umol) and t-BuONa (714.12 mg, 7.43 mmol). The mixture was degassed and purged with N₂ three times and the resulting mixture was heated to 100° C. and stirred for 18 hours. The mixture was cooled to 45° C. and diluted with ethyl acetate (10 mL). 3-Mercaptopropyl-functionalized silica gel (200 mg) was added and the mixture was stirred for 2 hours at 45° C. The mixture was filtered through a Celite pad and the filter cake was rinsed with ethyl acetate (10 mL*3). The combined filtrate was concentrated under reduced pressure and the residue was diluted with water (10 mL) and extracted with ethyl acetate (20 mL*2). The combined organic phase was washed with brine (10 mL), dried over anhydrous Na₂SO₄, filtered and the filtrate was concentrated. The residue was purified by column chromatography (SiO₂, petroleum ether:ethyl acetate=10:1 to 1:1) to give 56b. MS mass calculated for [M+H]⁺(C₂₁H₂₁Cl₂N₃O₂S) requires m/z, 450.1/452.1, LCMS found m/z, 450.0/452.0; ¹H NMR (400 MHz, CHLOROFORM-d) δ=8.18 (s, 1H), 7.41-7.27 (m, 3H), 6.97 (s, 1H), 4.34 (s, 2H), 3.44 (td, J=3.7, 7.3 Hz, 1H), 3.08 (ddd, J=3.7, 7.6, 11.7 Hz, 2H), 2.88 (ddd, J=3.7, 7.9, 11.9 Hz, 2H), 2.20-2.09 (m, 1H), 1.86-1.73 (m, 2H), 1.69-1.58 (m, 2H), 1.32-1.23 (m, 2H), 1.19-1.08 (m, 2H).

4-(((1-(2-bromothiazol-5-yl)piperidin-4-yl)oxy)methyl)-5-cyclopropyl-3-(2,6-dichlorophenyl)isoxazole (56c). To a solution of 5-cyclopropyl-3-(2,6-dichlorophenyl)-4-(((1-(thiazol-5-yl)piperidin-4-yl)oxy)methyl)isoxazole (56b) (300 mg, 666.10 umol) in THE (2 mL) was added LDA (2 M, 499.58 uL) at −78° C. and stirred for 10 minutes, then CBr₄ (242.99 mg, 732.71 umol) dissolved in THE (2 mL) was added dropwise at this temperature and the mixture was stirred for another 2 hours at 0° C. The reaction mixture was quenched with saturated ammonium chloride solution (5 mL) and extracted with ethyl acetate (10 mL*2). The combined organic layer was washed with brine (10 mL), dried over anhydrous Na₂SO₄, filtered and the filtrate was concentrated. The residue was purified by column chromatography (SiO₂, petroleum ether:ethyl acetate=50:1 to 5:1) to give 56c. MS mass calculated for [M+H]⁺(C₂₁H₂₀BrCl₂N₃O₂S) requires m/z, 530.0/528.0, LCMS found m/z, 530.0/528.0; ¹H NMR (400 MHz, CHLOROFORM-d) δ=7.43-7.35 (m, 2H), 7.33-7.28 (m, 1H), 6.64 (s, 1H), 4.33 (s, 2H), 3.44 (td, J=3.4, 6.8 Hz, 1H), 3.00 (tdd, J=3.9, 7.8, 11.7 Hz, 2H), 2.82 (ddd, J=3.9, 7.6, 12.0 Hz, 2H), 2.18-2.09 (m, 1H), 1.77 (tdd, J=3.7, 8.3, 12.7 Hz, 2H), 1.68-1.56 (m, 2H), 1.32-1.23 (m, 2H), 1.19-1.07 (m, 2H).

5-(4-((5-cyclopropyl-3-(2,6-dichlorophenyl)isoxazol-4-yl)methoxy)piperidin-1-yl)thiazole-2-carbonitrile (56d). To a solution of 4-(((1-(2-bromothiazol-5-yl)piperidin-4-yl)oxy)methyl)-5-cyclopropyl-3-(2,6-dichlorophenyl)isoxazole (56c) (200 mg, 377.87 umol) in toluene (3 mL) was added K₄[Fe(CN)₆] (55.67 mg, 151.15 umol), CuI (21.59 mg, 113.36 umol) and 1-methylimidazole (62.05 mg, 755.75 umol, 60.24 uL) in a sealed tube, then bubbled with N₂ for 1 minute The resulting mixture was heated to 160° C. and stirred for 5 hours. The reaction mixture was concentrated under reduced pressure. The residue was purified by prep-TLC (SiO₂, petroleum ether:ethyl acetate=1:2) to give 56d. MS mass calculated for [M+H]⁺(C₂₂H₂₀Cl₂N₄O₂S) requires m/z, 475.1/477.1 LCMS found m/z, 475.0/477.0; ¹H NMR (400 MHz, CHLOROFORM-d) δ=7.45-7.35 (m, 2H), 7.33-7.27 (m, 1H), 6.99 (s, 1H), 4.34 (s, 2H), 3.60-3.49 (m, 1H), 3.24-3.11 (m, 2H), 3.09-3.00 (m, 2H), 2.17-2.08 (m, 1H), 1.85-1.74 (m, 2H), 1.73-1.63 (m, 2H), 1.28-1.23 (m, 2H), 1.19-1.09 (m, 2H).

(Z)-5-(4-((5-cyclopropyl-3-(2,6-dichlorophenyl)isoxazol-4-yl)methoxy)piperidin-1-yl)-N′-hydroxythiazole-2-carboximidamide (56e). To a solution of 5-(4-((5-cyclopropyl-3-(2,6-dichlorophenyl)isoxazol-4-yl)methoxy)piperidin-1-yl)thiazole-2-carbonitrile (56d) (40 mg, 84.14 umol) in ethanol (0.5 mL) was added hydroxylamine (0.2 mL, 50% in water) and the mixture was stirred for 4 hours at 80° C. The reaction mixture was diluted with water (5 mL) and extracted with dichloromethane (10 mL*2). The combined organic phase was washed with brine (10 mL), dried over anhydrous Na₂SO₄, filtered and the filtrate was concentrated under reduced pressure. The residue was purified by prep-TLC (SiO₂, dichloromethane:methanol=10:1) to give 56e. MS mass calculated for [M+H]⁺(C₂₂H₂₃Cl₂N₅O₃S) requires m/z, 508.1/510.1, LCMS found m/z, 508.0/510.0.

### 3-(5-(4-((5-cyclopropyl-3-(2,6-dichlorophenyl)isoxazol-4-yl)methoxy)piperidin-1-yl)thiazol-2-yl)-1,2,4-oxadiazol-5(4H)-one (Compound 56)

To a solution of (Z)-5-(4-((5-cyclopropyl-3-(2,6-dichlorophenyl)isoxazol-4-yl)methoxy)piperidin-1-yl)-N′-hydroxythiazole-2-carboximidamide (56e) (25 mg, 49.17 umol) in ethanol (1 mL) was added diethyl carbonate (348.52 mg, 2.95 mmol, 357.46 uL) and CH₃ONa (15.94 mg, 295.03 umol) in a sealed tube. The mixture was heated to 100° C. and stirred for 2 hours. The reaction mixture was diluted with water (5 mL) and was extracted with dichloromethane (10 mL*2). The combined organic phase was washed with brine (5 mL*4), dried over anhydrous Na₂SO₄, filtered and the filtrate was concentrated under reduced pressure. The residue was purified by prep-HPLC (TFA condition; column: Nano-micro Kromasil C18 100*40 mm 10 um; mobile phase: [water (0.10% TFA)-ACN]; B %: 25%-57%, 8 min) and repurified by prep-TLC (SiO₂, dichloromethane:methanol=10:1) to give Compound 56. MS mass calculated for [M+H]⁺(C₂₃H₂₁Cl₂N₅O₄S) requires m/z, 534.1/536.1, LCMS found m/z, 534.2/536.1; ¹H NMR (400 MHz, CHLOROFORM-d) δ=7.51-7.32 (m, 3H), 7.09 (br s, 1H), 4.43 (s, 2H), 3.63 (br s, 1H), 3.37-3.24 (m, 2H), 3.15 (br d, J=5.3 Hz, 2H), 2.17 (br s, 1H), 1.85 (br s, 2H), 1.74 (br s, 2H), 1.33 (br s, 2H), 1.23 (br s, 2H).

### Example 57

### 6-(4-(4-((5-cyclopropyl-3-(2-(trifluoromethoxy)phenyl)isoxazol-4-yl)methoxy)piperidin-1-yl)phenyl)-1,2,4-triazine-3,5(2H,4H)-dione

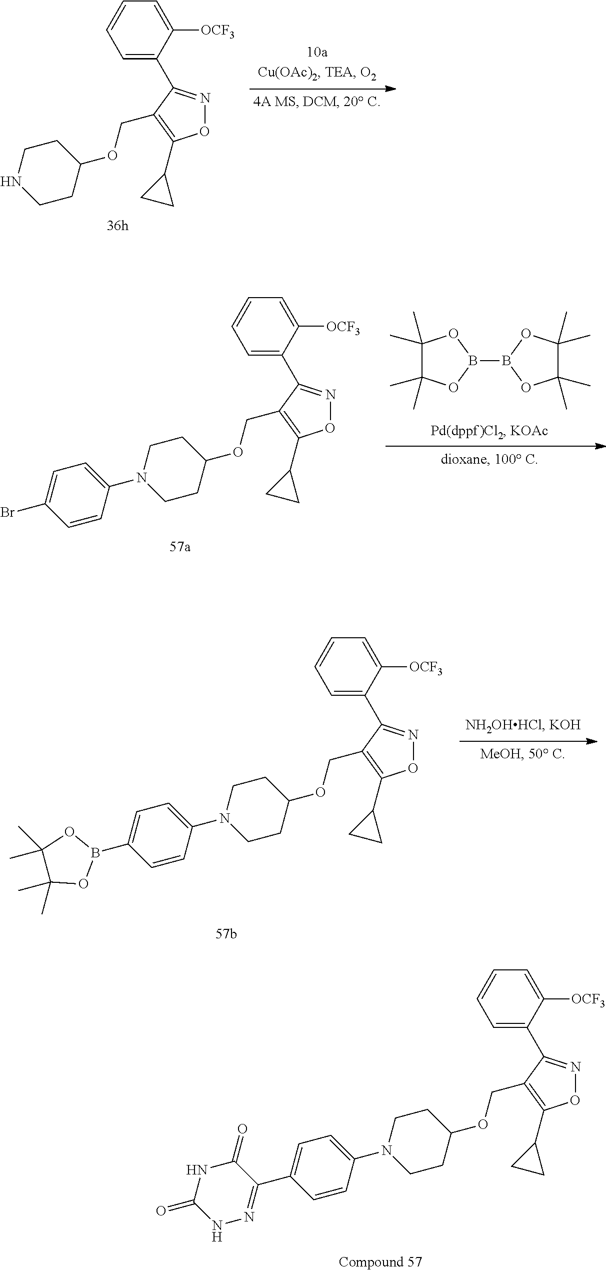

4-(((1-(4-bromophenyl)piperidin-4-yl)oxy)methyl)-5-cyclopropyl-3-(2-(trifluoromethoxy)phenyl)isoxazole (57a). To a solution of 5-cyclopropyl-4-((piperidin-4-yloxy)methyl)-3-(2-(trifluoromethoxy)phenyl)isoxazole (36h) (80 mg, 209.22 umol) in dichloromethane (4 mL) was added (4-bromophenyl)boronic acid (10a) (63.02 mg, 313.83 umol), Cu(OAc)₂ (45.60 mg, 251.06 umol), TEA (42.34 mg, 418.44 umol, 58.24 uL) and molecular sieve 4 A (20 mg) at 20° C. The mixture was stirred at 20° C. for 16 hours under O₂ ballo and was diluted with ethyl acetate (30 mL) and filtered. The filtrate was washed with H₂O (10 mL), brine (10 mL), dried over anhydrous Na₂SO₄, filtered and the filtrate was concentrated under reduced pressure to give a residue. The residue was purified by prep-TLC to give 57a. MS mass calculated for [M+H]⁺(C₂₅H₂₄BrF₃N₂O₃) requires m/z, 537.0, LCMS found m/z, 537.1; ¹H NMR (400 MHz, CHLOROFORM-d) δ=7.58 (dd, J=1.4, 7.9 Hz, 1H), 7.54-7.47 (m, 1H), 7.42-7.35 (m, 2H), 7.31 (d, J=9.1 Hz, 2H), 6.75 (d, J=8.9 Hz, 2H), 4.40 (s, 2H), 3.42 (tt, J=3.9, 8.1 Hz, 1H), 3.36-3.27 (m, 2H), 2.84 (ddd, J=3.2, 9.2, 12.3 Hz, 2H), 2.20-2.10 (m, 1H), 1.84 (br dd, J=3.0, 15.4 Hz, 2H), 1.64-1.56 (m, 2H), 1.28-1.21 (m, 2H), 1.15-1.08 (m, 2H).

5-cyclopropyl-4-(((1-(4-(4,4,5,5-tetramethyl-1,3,2-dioxaborolan-2-yl)phenyl)piperidin-4-yl)oxy)methyl)-3-(2-(trifluoromethoxy)phenyl)isoxazole (57b). To a solution of 4-(((1-(4-bromophenyl)piperidin-4-yl)oxy)methyl)-5-cyclopropyl-3-(2-(trifluoromethoxy)phenyl)isoxazole (57a) (90 mg, 167.48 umol) in 1,4-dioxane (5 mL) was added 4,4,5,5-tetramethyl-2-(4,4,5,5-tetramethyl-1,3,2-dioxaborolan-2-yl)-1,3,2-dioxaborolane (127.59 mg, 502.45 umol), Pd(dppf)Cl₂ (12.25 mg, 16.75 umol) and KOAc (32.87 mg, 334.97 umol) at 20° C. The resulting mixture was degassed and purged with N₂ three times and heated to 100° C. for 16 hours. The reaction mixture was diluted with ethyl acetate (30 mL) and filtered and the filtrate was washed with H₂O (10 mL), brine (10 mL), dried over anhydrous Na₂SO₄ and filtered. The filtrate was concentrated under reduced pressure to give a residue. The residue was purified by prep-TLC to give 57b. MS mass calculated for [M+H]⁺(C₃₁H₃₆BF₃N₂O₅) requires m/z, 585.3/586.3, LCMS found m/z, 585.0/586.1.

6-(4-(4-((5-cyclopropyl-3-(2-(trifluoromethoxy)phenyl)isoxazol-4-yl)methoxy)piperidin-1-yl)phenyl)-1,2,4-triazine-3,5(2H,4H)-dione (Compound 57). To a solution of 5-cyclopropyl-4-(((1-(4-(4,4,5,5-tetramethyl-1,3,2-dioxaborolan-2-yl)phenyl)piperidin-4-yl)oxy)methyl)-3-(2-(trifluoromethoxy)phenyl)isoxazole (57b) (45 mg, 77.00 umol) and 6-bromo-1,2,4-triazine-3,5(2H,4H)-dione (10d) (44.34 mg, 230.99 umol) in THE (3.2 mL) and H₂O (0.8 mL) was added ditert-butyl (cyclopentyl)phosphane; dichloropalladium; iron (5.02 mg, 7.70 umol) and K₃PO₄ (32.69 mg, 154.00 umol, 2 eq) at 20° C. The resulting mixture was degassed and purged with N₂ three times and was heated to 80° C. and stirred for 16 hours under N₂ atmosphere. Water (10 mL) was added to the mixture and the resulting mixture was extracted with ethyl acetate (10 mL*2). The combined organic phase was washed with brine (20 mL), dried with anhydrous Na₂SO₄, filtered and the filtrate was concentrated under reduced pressure. The residue was purified by prep-HPLC (column: Nano-micro Kromasil C18 100*40 mm 10 um; mobile phase: [water (0.1% TFA)-ACN]; B %: 25%-55%, 8 min) and lyophilized to give Compound 57. MS mass calculated for [M+H]⁺(C₂₈H₂₆F₃N₅O₅) requires m/z, 570.2, LCMS found m/z, 570.2; ¹H NMR (400 MHz, CHLOROFORM-d) δ=10.08 (br s, 1H), 9.25 (br s, 1H), 8.05 (br d, J=8.4 Hz, 2H), 7.58 (br d, J=7.5 Hz, 1H), 7.54-7.47 (m, 1H), 7.44-7.34 (m, 2H), 7.33-7.28 (m, 1H), 7.26-7.20 (m, 1H), 4.45 (s, 2H), 4.35-3.80 (m, 1H), 3.63 (br s, 1H), 3.44-3.31 (m, 2H), 3.26 (br s, 2H), 2.12 (br t, J=8.3 Hz, 3H), 1.79 (br d, J=11.4 Hz, 2H), 1.30-1.21 (m, 2H), 1.18-1.09 (m, 2H).

### Example 58

### 3-(3-(4-((5-cyclopropyl-3-(2,6-dichlorophenyl)isoxazol-4-yl)methoxy)piperidin-1-yl)-1H-pyrazol-5-yl)-1,2,4-oxadiazol-5(4H)-one

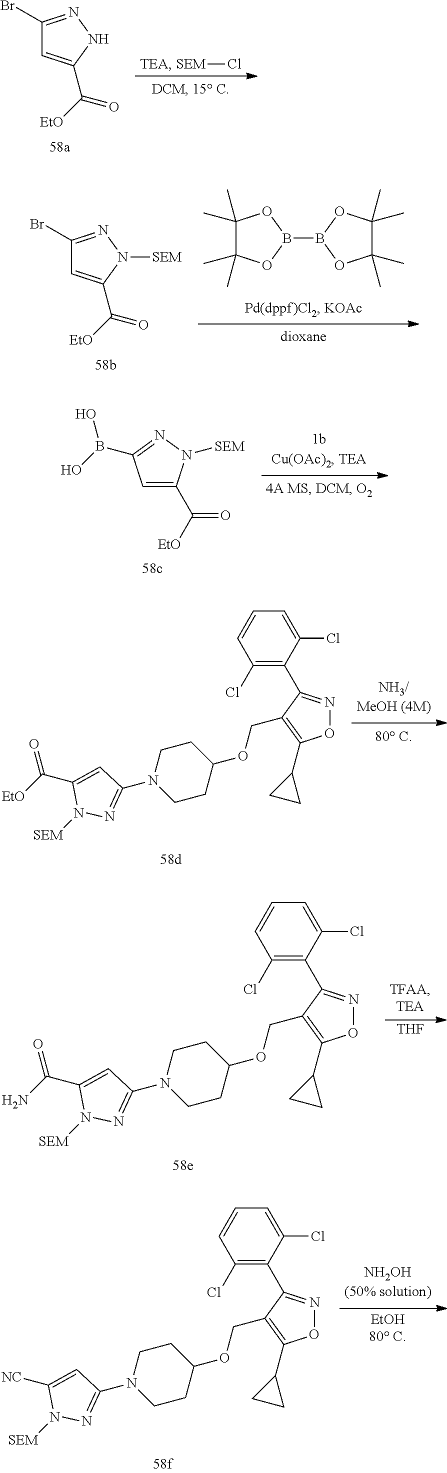

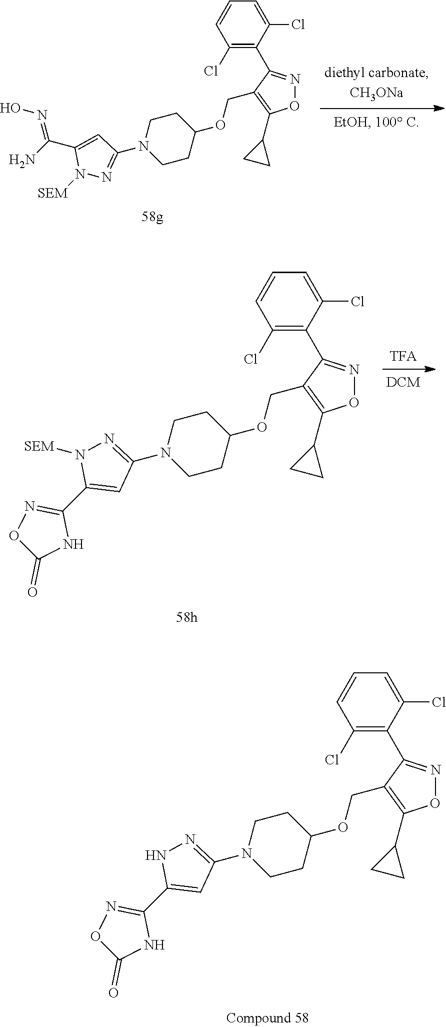 [SMILES: C[Si](C)(C)CCOCn1nc(N2CCC(OCc3c(-c4c(Cl)cccc4Cl)noc3C3CC3)CC2)cc1/C(N)=N/O ; C[Si](C)(C)CCOCn1nc(N2CCC(OCc3c(-c4c(Cl)cccc4Cl)noc3C3CC3)CC2)cc1-c1noc(=O)[nH]1 ; O=c1[nH]c(-c2cc(N3CCC(OCc4c(-c5c(Cl)cccc5Cl)noc4C4CC4)CC3)n[nH]2)no1]

Ethyl 3-bromo-1-((2-(trimethylsilyl)ethoxy)methyl)-1H-pyrazole-5-carboxylate (58b). To a solution of ethyl 3-bromo-1H-pyrazole-5-carboxylate (58a) (0.88 g, 4.02 mmol) in dichloromethane (20 mL) was added TEA (609.81 mg, 6.03 mmol, 838.80 uL) and SEM-Cl (703.31 mg, 4.22 mmol, 746.61 uL) dropwise at 15° C. and the mixture was stirred for 18 hours. The reaction mixture was diluted with water (10 mL) and extracted with ethyl acetate (15 mL*3). The combined organic phase was washed with brine (10 mL), dried over anhydrous Na₂SO₄, filtered and the filtrate was concentrated under reduced pressure. The residue was purified by column chromatography (SiO₂, petroleum ether:ethyl acetate=50:1 to 10:1) to give 58b. MS mass calculated for [M+H]⁺(C₁₂H₂₁BrN₂O₃Si) requires m/z, 351.1/349.1, LCMS found m/z, 291.2/293.2; ¹H NMR (400 MHz, CHLOROFORM-d) δ=7.64 (s, 1H), 6.18 (s, 2H), 4.74 (q, J=7.1 Hz, 2H), 3.99 (t, 2H), 1.75 (t, J=7.2 Hz, 3H), 1.31-1.23 (m, 2H), 0.35 (s, 9H).

(5-(ethoxycarbonyl)-1-((2-(trimethylsilyl)ethoxy)methyl)-1H-pyrazol-3-yl)boronic acid (58c). To a solution of ethyl 3-bromo-1-((2-(trimethylsilyl)ethoxy)methyl)-1H-pyrazole-5-carboxylate (58b) (900 mg, 2.58 mmol) and 4,4,5,5-tetramethyl-2-(4,4,5,5-tetramethyl-1,3,2-dioxaborolan-2-yl)-1,3,2-dioxaborolane (3.27 g, 12.88 mmol) in 1,4-DIXOANE (20 mL) was added Pd(dppf)Cl₂ (377.07 mg, 515.32 umol) and KOAc (505.74 mg, 5.15 mmol). The reaction mixture was degassed and purged with N₂ three times and was heated to 100° C. and stirred for 18 hours. The reaction mixture was diluted with ethyl acetate (5 mL) and 3-mercaptopropyl-functionalized silica gel (500 mg) was added and the mixture was stirred at 45° C. for 2 hours. The mixture was filtered through a Celite pad and the filter cake was rinsed with ethyl acetate (5 mL*3). The combined filtrate was concentrated under reduced pressure. The residue was purified by prep-HPLC (TFA condition; column: Phenomenex luna c18 250 mm*100 mm*15 um; mobile phase: [water (0.10% TFA)-ACN]; B %: 35%-65%, 20 min) and lyophilized to give 58c. MS mass calculated for [M+H]⁺(C₁₂H₂₃BN₂O₅Si) requires m/z, 315.2/314.2, LCMS found m/z, 315.3/314.3; ¹H NMR (400 MHz, CHLOROFORM-d) δ=7.30 (s, 1H), 6.01-5.83 (m, 2H), 4.44-4.31 (m, 2H), 3.69-3.52 (m, 2H), 1.45-1.34 (m, 3H), 0.97-0.84 (m, 2H), 0.10-−0.19 (m, 9H).

Ethyl 3-(4-((5-cyclopropyl-3-(2,6-dichlorophenyl)isoxazol-4-yl)methoxy)piperidin-1-yl)-1-((2-(trimethylsilyl)ethoxy)methyl)-1H-pyrazole-5-carboxylate (58d). To a mixture of (5-(ethoxycarbonyl)-1-((2-(trimethylsilyl)ethoxy)methyl)-1H-pyrazol-3-yl)boronic acid (58c) (200 mg, 636.50 umol) and 5-cyclopropyl-3-(2,6-dichlorophenyl)-4-((piperidin-4-yloxy)methyl)isoxazole hydrochloride (1b) (268.83 mg, 731.98 umol) in dichloromethane (5 mL) was added Cu(OAc)₂ (115.61 mg, 636.50 umol), TEA (128.81 mg, 1.27 mmol, 177.19 uL) and Molecular sieve 4 A (30 mg) at 15° C. The resulting suspension was degassed and purged with O₂ 3 times and was warmed to 25° C. and stirred for 20 hours under O₂ ballon. The reaction mixture was filtered through a Celite pad and the filter cake was rinsed with dichloromethane (10 mL*2). The combined filtrate was concentrated under reduced pressure. The residue was purified by column chromatography (SiO₂, petroleum ether:ethyl acetate=10:1) to give 58d. MS mass calculated for [M+H]⁺(C₃₀H₄₀Cl₂N₄O₅Si) requires m/z, 635.2/637.2 LCMS found m/z, 635.2/637.2; ¹H NMR (400 MHz, CHLOROFORM-d) δ=7.44-7.37 (m, 2H), 7.35-7.28 (m, 1H), 6.28 (s, 1H), 5.68 (s, 2H), 4.40-4.23 (m, 4H), 3.68-3.51 (m, 2H), 3.45-3.30 (m, 1H), 3.45-3.30 (m, 1H), 3.45-3.30 (m, 1H), 2.84 (br t, J=9.3 Hz, 2H), 2.23-2.11 (m, 1H), 1.75 (br s, 1H), 1.83-1.69 (m, 1H), 1.54-1.44 (m, 2H), 1.37 (t, J=7.1 Hz, 3H), 1.32-1.23 (m, 3H), 1.13 (br dd, J=2.7, 8.3 Hz, 1H), 1.18-1.07 (m, 1H), 0.94-0.86 (m, 2H), 0.03 (s, 9H).

3-(4-((5-cyclopropyl-3-(2,6-dichlorophenyl)isoxazol-4-yl)methoxy)piperidin-1-yl)-1-((2-(trimethylsilyl)ethoxy)methyl)-1H-pyrazole-5-carboxamide (58e). A solution of ethyl 3-(4-((5-cyclopropyl-3-(2,6-dichlorophenyl)isoxazol-4-yl)methoxy)piperidin-1-yl)-1-((2-(trimethylsilyl)ethoxy)methyl)-1H-pyrazole-5-carboxylate (58d) (100 mg, 157.32 umol) in NH₃/methanol (8 mL, 4 M) was stirred at 80° C. for 18 hours in sealed tube. The reaction mixture was concentrated under reduced pressure. The residue was purified by prep-TLC (SiO₂, dichloromethane:methanol=10:1) to give 58e. MS mass calculated for [M+H]⁺(C₂₈H₃₇Cl₂N₅O₄Si) requires m/z, 606.2/608.2, LCMS found m/z, 606.2/608.2; ¹H NMR (400 MHz, CHLOROFORM-d) δ=7.44-7.37 (m, 2H), 7.36-7.29 (m, 1H), 7.01 (br s, 1H), 6.20 (s, 1H), 5.55 (s, 2H), 4.34 (s, 2H), 3.74-3.59 (m, 2H), 3.47-3.29 (m, 3H), 2.93-2.74 (m, 2H), 2.21-2.10 (m, 1H), 1.83-1.70 (m, 2H), 1.51 (br dd, J=4.2, 8.7 Hz, 2H), 1.31-1.24 (m, 3H), 1.17-1.08 (m, 2H), 0.99-0.86 (m, 2H), 0.06-−0.07 (m, 9H).

3-(4-((5-cyclopropyl-3-(2,6-dichlorophenyl)isoxazol-4-yl)methoxy)piperidin-1-yl)-1-((2-(trimethylsilyl)ethoxy)methyl)-1H-pyrazole-5-carbonitrile (58f). To a solution of 3-(4-((5-cyclopropyl-3-(2,6-dichlorophenyl)isoxazol-4-yl)methoxy)piperidin-1-yl)-1-((2-(trimethylsilyl)ethoxy)methyl)-1H-pyrazole-5-carboxamide (58e) (40 mg, 65.94 umol) in THF (1 mL) was added TEA (40.03 mg, 395.64 umol, 55.07 uL) at 0° C., then TFAA (41.55 mg, 197.82 umol, 27.52 uL) was added and the mixture was stirred for 10 minutes at 15° C. The reaction mixture was quenched with saturated sodium bicarbonate solution (5 mL) and extracted with ethyl acetate (10 mL*2). The combined organic phase was washed with brine (10 mL), dried over anhydrous Na₂SO₄, filtered and the filtrate was concentrated under reduced pressure. The residue was purified by prep-TLC (SiO₂, petroleum ether:ethyl acetate=2:1) to give 58f. MS mass calculated for [M+H]⁺(C₂₈H₃₅Cl₂N₅O₃Si) requires m/z, 588.2/590.2, LCMS found m/z, 588.1/590.2; ¹H NMR (400 MHz, CHLOROFORM-d) δ=7.45-7.36 (m, 2H), 7.35-7.28 (m, 1H), 6.17 (s, 1H), 5.39 (s, 2H), 4.34 (s, 2H), 3.68-3.54 (m, 2H), 3.44-3.26 (m, 3H), 2.88 (ddd, J=3.4, 9.0, 12.5 Hz, 2H), 2.22-2.09 (m, 1H), 1.74 (br s, 2H), 1.62-1.42 (m, 3H), 1.32-1.19 (m, 3H), 1.17-1.06 (m, 2H), 1.00-0.85 (m, 2H), 0.00 (s, 9H).

(Z)-3-(4-((5-cyclopropyl-3-(2,6-dichlorophenyl)isoxazol-4-yl)methoxy)piperidin-1-yl)-N′-hydroxy-1-((2-(trimethylsilyl)ethoxy)methyl)-1H-pyrazole-5-carboximidamide (58g). To a solution of 3-(4-((5-cyclopropyl-3-(2,6-dichlorophenyl)isoxazol-4-yl)methoxy)piperidin-1-yl)-1-((2-(trimethylsilyl)ethoxy)methyl)-1H-pyrazole-5-carbonitrile (58f) (50 mg, 84.95 umol) in ethanol (1 mL) was added hydroxylamine (0.5 mL, 50% in water) in one portion. The mixture was heated to 80° C. and stirred for 4 hours. The reaction mixture was diluted with water (5 mL) and extracted with dichloromethane (10 mL*2). The combined organic phase was washed with brine (5 mL), dried over anhydrous Na₂SO₄, filtered and the filtrate was concentrated under reduced pressure. The residue was purified by prep-TLC (SiO₂, dichloromethane:methanol=10:1) to give 58g. MS mass calculated for [M+H]⁺(C₂₈H₃₈Cl₂N₆O₄Si) requires m/z, 621.2/623.2, LCMS found m/z, 621.2/623.2; ¹H NMR (400 MHz, CHLOROFORM-d) δ=7.44-7.36 (m, 2H), 7.35-7.29 (m, 1H), 6.80 (br s, 1H), 5.97 (s, 1H), 5.45 (s, 2H), 4.34 (s, 2H), 3.71-3.61 (m, 2H), 3.43-3.30 (m, 3H), 2.87-2.74 (m, 2H), 2.24-2.09 (m, 1H), 1.81-1.68 (m, 2H), 1.56-1.44 (m, 2H), 1.29-1.23 (m, 2H), 1.18-1.07 (m, 2H), 0.97-0.85 (m, 2H),−0.01 (s, 9H).

3-(3-(4-((5-cyclopropyl-3-(2,6-dichlorophenyl)isoxazol-4-yl)methoxy)piperidin-1-yl)-1-((2-(trimethylsilyl)ethoxy)methyl)-1H-pyrazol-5-yl)-1,2,4-oxadiazol-5(4H)-one (58h). To a solution of (Z)-3-(4-((5-cyclopropyl-3-(2,6-dichlorophenyl)isoxazol-4-yl)methoxy)piperidin-1-yl)-N′-hydroxy-1-((2-(trimethylsilyl)ethoxy)methyl)-1H-pyrazole-5-carboximidamide (58g) (45 mg, 72.39 umol) in ethanol (1 mL) was added CH₃ONa (78.21 mg, 434.34 umol, 30% in MeOH) and diethyl carbonate (513.09 mg, 4.34 mmol, 526.25 uL) in a sealed tube. The mixture was heated to 100° C. and stirred for 2 hours. The reaction mixture was concentrated under reduced pressure and diluted with water (5 mL) and extracted with ethyl acetate 30 mL (10 mL*3). The combined organic layers were washed with brine (10 mL), dried over anhydrous Na₂SO₄, filtered and the filtrate was concentrated under reduced pressure to give a residue. The residue was purified by prep-TLC (SiO₂, dichloromethane:methanol=10:1) to give 58h. MS mass calculated for [M+H]⁺(C₂₉H₃₆Cl₂N₆O₅Si) requires m/z, 647.2/649.2, LCMS found m/z,/647.2/649.2; ¹H NMR (400 MHz, CHLOROFORM-d) δ=7.44-7.37 (m, 2H), 7.36-7.29 (m, 1H), 6.31 (br s, 1H), 5.45 (s, 2H), 4.34 (s, 2H), 3.72-3.63 (m, 2H), 3.46-3.29 (m, 3H), 2.87 (br t, J=9.0 Hz, 2H), 2.22-2.10 (m, 1H), 1.75 (br s, 2H), 1.57-1.47 (m, 2H), 1.30-1.25 (m, 2H), 1.17-1.06 (m, 2H), 1.03-0.90 (m, 2H), 0.10-0.04 (m, 9H).

3-(3-(4-((5-cyclopropyl-3-(2,6-dichlorophenyl)isoxazol-4-yl)methoxy)piperidin-1-yl)-1H-pyrazol-5-yl)-1,2,4-oxadiazol-5(4H)-one (Compound 58). To a solution of 3-(3-(4-((5-cyclopropyl-3-(2,6-dichlorophenyl)isoxazol-4-yl)methoxy)piperidin-1-yl)-1-((2-(trimethylsilyl)ethoxy)methyl)-1H-pyrazol-5-yl)-1,2,4-oxadiazol-5(4H)-one (58h) (8 mg, 12.35 umol) in dichloromethane (0.2 mL) was added TFA (0.1 mL) and stirred for 2 hours at 15° C. The reaction mixture was concentrated under reduced pressure. The residue was purified by prep-HPLC (TFA condition; column: Phenomenex luna C18 80*40 mm*3 um; mobile phase: [water (0.1% TFA)-ACN]; B %: 40%-60%, 12 min) and lyophilized to give Compound 58. MS mass calculated for [M+H]⁺(C₂₃H₂₂Cl₂N₆O₄) requires m/z, 517.1/519.1, LCMS found m/z, 517.1/519.1; ¹H NMR (400 MHz, CHLOROFORM-d) δ=13.40 (br s, 1H), 7.44-7.37 (m, 2H), 7.35-7.29 (m, 1H), 5.99 (s, 1H), 4.34 (s, 2H), 3.49 (td, J=3.4, 6.5 Hz, 1H), 3.21-3.08 (m, 2H), 2.97 (ddd, J=3.9, 7.1, 11.5 Hz, 2H), 2.14 (tt, J=5.0, 8.5 Hz, 1H), 1.82-1.75 (m, 2H), 1.65 (br dd, J=6.8, 10.8 Hz, 2H), 1.34-1.24 (m, 2H), 1.19-1.09 (m, 2H).

### Example 59

### 5-(6-(4-((5-cyclopropyl-3-(2,6-dichlorophenyl)isoxazol-4-yl)methoxy)piperidin-1-yl)pyridin-3-yl)isoxazol-3(2H)-one

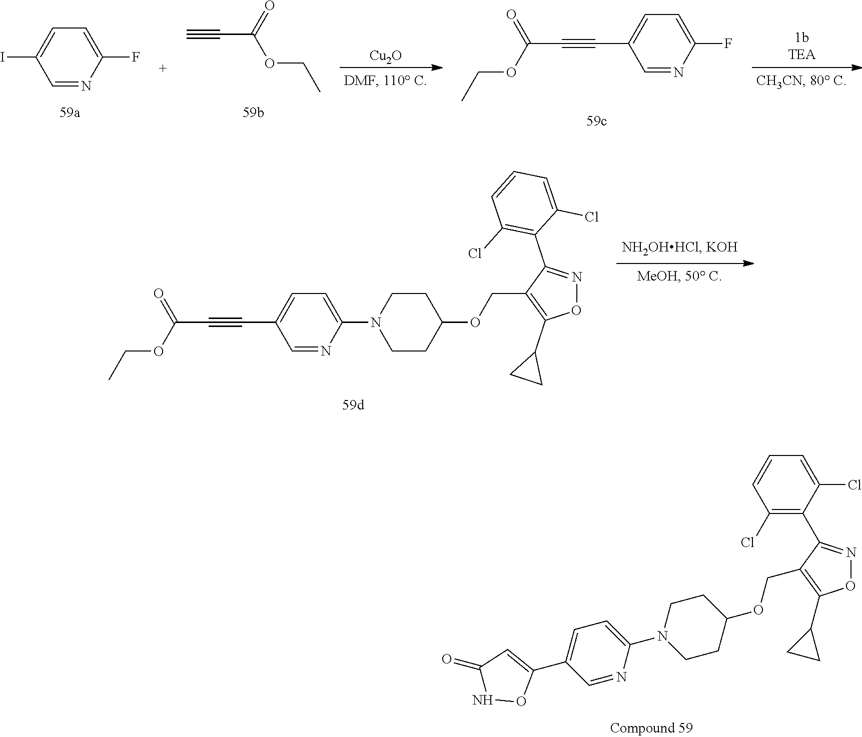

Ethyl 3-(6-fluoropyridin-3-yl)propiolate (59c). To a solution of 2-fluoro-5-iodopyridine (59a) (0.3 g, 1.35 mmol) and Ethyl propiolate (59b) (395.94 mg, 4.04 mmol, 395.94 uL) in DMF (3 mL) was added Cu₂O (19.25 mg, 134.54 umol, 13.75 uL) under N₂. The resulting mixture was degassed and purged with N₂ 3 times and was heated to 110° C. and stirred for 18 hours. The reaction mixture was diluted with water (5 mL) and extracted with ethyl acetate (10 mL*2). The combined organic phase was washed with brine (10 mL), dried over anhydrous Na₂SO₄, filtered and the filtrate was concentrated under reduced pressure. The residue was purified by column chromatography (SiO₂, petroleum ether:ethyl acetate=100:1 to 50:1) to give 59c. ¹H NMR (400 MHz, CHLOROFORM-d) δ=8.47 (d, J=2.4 Hz, 1H), 7.98 (ddd, J=2.4, 7.3, 8.3 Hz, 1H), 6.99 (dd, J=2.9, 8.3 Hz, 1H), 4.32 (q, J=7.3 Hz, 2H), 1.37 (t, J=7.3 Hz, 3H).

Ethyl 3-(6-(4-((5-cyclopropyl-3-(2,6-dichlorophenyl)isoxazol-4-yl)methoxy)piperidin-1-yl)pyridin-3-yl)propiolate (59d). To a solution of 5-cyclopropyl-3-(2,6-dichlorophenyl)-4-((piperidin-4-yloxy)methyl)isoxazole hydrochloride (1b) (209.00 mg, 517.67 umol) in CH₃CN (1 mL) was added TEA (104.77 mg, 1.04 mmol, 144.11 uL) and ethyl 3-(6-fluoropyridin-3-yl)propiolate (59c) (100 mg, 517.67 umol) and the mixture was heated to 80° C. and stirred for 23 hours. The reaction mixture was diluted with water (5 mL) and was extracted with ethyl acetate (10 mL*2). The combined organic phase was washed with brine (10 mL), dried over anhydrous Na₂SO₄, filtered and the filtrate was concentrated under reduced pressure. The residue was purified by prep-TLC (SiO₂, petroleum ether:ethyl acetate=3:1) to give 59d. MS mass calculated for [M+H]⁺(C₂₈H₂₇Cl₂N₃O₄) requires m/z, 540.1/542.1, LCMS found m/z, 540.2/542.1; ¹H NMR (400 MHz, CHLOROFORM-d) δ=8.37 (d, J=2.0 Hz, 1H), 7.61-7.55 (m, 1H), 7.41 (d, J=1.5 Hz, 1H), 7.40 (s, 1H), 7.34-7.28 (m, 1H), 6.54 (d, J=8.8 Hz, 1H), 4.35 (s, 2H), 4.29 (q, J=7.3 Hz, 2H), 3.78-3.65 (m, 2H), 3.51 (tt, J=3.7, 7.3 Hz, 1H), 3.38-3.26 (m, 2H), 2.15 (tt, J=5.1, 8.4 Hz, 1H), 1.77-1.66 (m, 2H), 1.54-1.43 (m, 2H), 1.39-1.32 (m, 3H), 1.31-1.27 (m, 2H), 1.18-1.09 (m, 2H).

5-(6-(4-((5-cyclopropyl-3-(2,6-dichlorophenyl)isoxazol-4-yl)methoxy)piperidin-1-yl)pyridin-3-yl)isoxazol-3(2H)-one (Compound 59). To a solution of ethyl 3-(6-(4-((5-cyclopropyl-3-(2,6-dichlorophenyl)isoxazol-4-yl)methoxy)piperidin-1-yl)pyridin-3-yl)propiolate (59d) (130 mg, 240.55 umol) in methanol (2 mL) was added hydroxylamine hydrochloride (167.16 mg, 2.41 mmol) and KOH (242.93 mg, 4.33 mmol) at 15° C. The mixture was heated to 50° C. and stirred for 18 hours and was concentrated under reduced pressure. The residue was diluted with water (5 mL) and extracted with ethyl acetate (10 mL*2). The combined organic phase was washed with brine (10 mL), dried over anhydrous Na₂SO₄, filtered and the filtrate was concentrated. The residue was purified by prep-TLC (SiO₂, dichloromethane:methanol=10:1) to give Compound 59. MS mass calculated for [M+H]⁺(C₂₆H₂₄Cl₂N₄O₄) requires m/z, 527.1/529.1, LCMS found m/z, 527.2/529.1; ¹H NMR (400 MHz, CHLOROFORM-d) δ=8.51 (d, J=2.4 Hz, 1H), 7.74 (dd, J=2.4, 8.8 Hz, 1H), 7.46-7.38 (m, 2H), 7.36-7.29 (m, 1H), 6.63 (d, J=8.8 Hz, 1H), 6.03 (br s, 1H), 4.36 (s, 2H), 3.82-3.67 (m, 2H), 3.51 (td, J=3.9, 7.5 Hz, 1H), 3.40-3.24 (m, 2H), 2.16 (tt, J=5.1, 8.4 Hz, 1H), 1.83-1.66 (m, 2H), 1.50 (dtd, J=3.9, 8.2, 12.5 Hz, 2H), 1.32-1.24 (m, 2H), 1.19-1.08 (m, 2H).

### Example 60

### 5-(6-(4-((5-cyclopropyl-3-(2-(trifluoromethoxy)phenyl)isoxazol-4-yl)methoxy)piperidin-1-yl)pyridin-3-yl)isoxazol-3(2H)-one

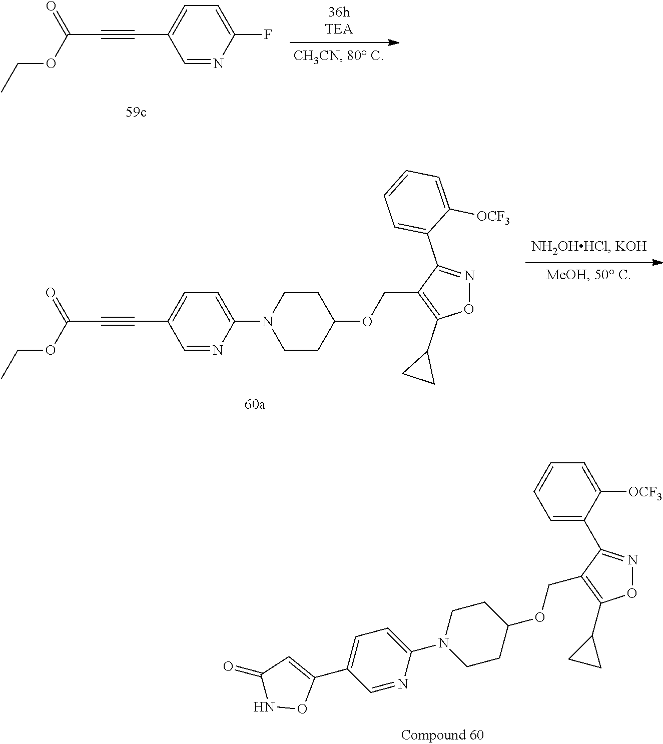 [SMILES: CCOC(=O)C#Cc1ccc(F)nc1 ; CCOC(=O)C#Cc1ccc(N2CCC(OCc3c(-c4ccccc4OC(F)(F)F)noc3C3CC3)CC2)nc1 ; O=c1cc(-c2ccc(N3CCC(OCc4c(-c5ccccc5OC(F)(F)F)noc4C4CC4)CC3)nc2)o[nH]1]

Ethyl 3-(6-(4-((5-cyclopropyl-3-(2-(trifluoromethoxy)phenyl)isoxazol-4-yl)methoxy)piperidin-1-yl)pyridin-3-yl)propiolate (60a). To a solution of ethyl 3-(6-fluoropyridin-3-yl)propiolate (59c) (50 mg, 258.83 umol) and 5-cyclopropyl-4-((piperidin-4-yloxy)methyl)-3-(2-(trifluoromethoxy)phenyl)isoxazole (36h) (98.97 mg, 258.83 umol) in acetonitrile (1 mL) was added TEA (52.38 mg, 517.67 umol, 72.05 uL). The mixture was heated to 80° C. and stirred for 24 hours. The reaction mixture was diluted with water (5 mL), extracted with ethyl acetate (10 mL*2). The combined organic phase was washed with brine (10 mL), dried over anhydrous Na₂SO₄, filtered and the filtrated was concentrated under reduced pressure. The residue was purified by prep-TLC (SiO₂, petroleum ether:ethyl acetate=3:1) to give 60a. MS mass calculated for [M+H]⁺(C₂₉H₂₈F₃N₃O₅) requires m/z, 556.2/557.2, LCMS found m/z, 556.1, 557.1; ¹H NMR (400 MHz, CHLOROFORM-d) δ=8.37 (d, J=1.5 Hz, 1H), 7.62-7.54 (m, 2H), 7.53-7.47 (m, 1H), 7.43-7.35 (m, 2H), 6.55 (d, J=8.8 Hz, 1H), 4.41 (s, 2H), 4.29 (q, J=7.3 Hz, 2H), 3.87-3.76 (m, 2H), 3.53 (tt, J=3.7, 7.6 Hz, 1H), 3.36-3.26 (m, 2H), 2.14 (tt, J=5.3, 8.4 Hz, 1H), 1.83-1.72 (m, 2H), 1.55-1.45 (m, 2H), 1.36 (t, J=7.1 Hz, 3H), 1.27-1.21 (m, 2H), 1.16-1.07 (m, 2H).

5-(6-(4-((5-cyclopropyl-3-(2-(trifluoromethoxy)phenyl)isoxazol-4-yl)methoxy)piperidin-1-yl)pyridin-3-yl)isoxazol-3(2H)-one (Compound 60). To a solution of ethyl 3-(6-(4-((5-cyclopropyl-3-(2-(trifluoromethoxy)phenyl)isoxazol-4-yl)methoxy)piperidin-1-yl)pyridin-3-yl)propiolate (60a) (80 mg, 144.00 umol) in methanol (2 mL) was added hydroxylamine hydrochloride (100.07 mg, 1.44 mmol) and KOH (145.43 mg, 2.59 mmol) at 15° C. The mixture was heated to 50° C. and stirred for 18 hours. The reaction mixture was concentrated under reduced pressure. The residue was diluted with water (5 mL) and extracted with ethyl acetate (10 mL*2). The combined organic phase was washed with brine (10 mL), dried over anhydrous Na₂SO₄, filtered and the filtrate was concentrated under reduced pressure. The residue was purified by prep-TLC (SiO₂, dichloromethane:methanol=10:1) to give Compound 60. MS mass calculated for [M+H]⁺(C₂₇H₂₅F₃N₄O₅) requires m/z, 543.2/544.2, LCMS found m/z, 543.2/544.2; ¹H NMR (400 MHz, CHLOROFORM-d) δ=8.51 (d, J=2.0 Hz, 1H), 7.74 (dd, J=2.0, 8.8 Hz, 1H), 7.58 (dd, J=1.7, 8.1 Hz, 1H), 7.55-7.46 (m, 1H), 7.45-7.32 (m, 2H), 6.65 (d, J=8.8 Hz, 1H), 6.03 (br s, 1H), 4.42 (s, 2H), 3.93-3.77 (m, 2H), 3.60-3.47 (m, 1H), 3.39-3.22 (m, 2H), 2.15 (tt, J=5.3, 8.4 Hz, 1H), 1.88-1.73 (m, 2H), 1.53 (dtd, J=3.7, 8.5, 12.7 Hz, 2H), 1.29-1.20 (m, 2H), 1.15-1.04 (m, 2H).

### Example 61

### 5-(6-(4-((5-cyclopropyl-3-(2-(trifluoromethoxy)phenyl)isoxazol-4-yl)methoxy)piperidin-1-yl)pyridin-3-yl)-1,3,4-oxadiazol-2(3H)-one

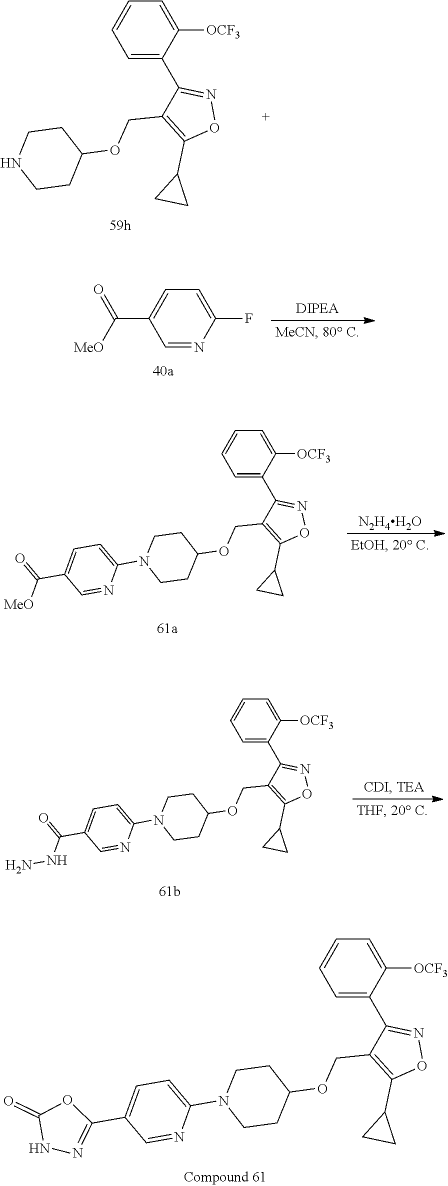

Methyl 6-(4-((5-cyclopropyl-3-(2-(trifluoromethoxy)phenyl)isoxazol-4-yl)methoxy)piperidin-1-yl)nicotinate (61a). To a solution of 5-cyclopropyl-4-((piperidin-4-yloxy)methyl)-3-(2-(trifluoromethoxy)phenyl)isoxazole (59h) (100 mg, 261.52 umol) and methyl 6-fluoronicotinate (40a) (81.14 mg, 523.05 umol) in acetonitrile (5 mL) was added DIPEA (169.00 mg, 1.31 mmol, 227.76 uL) at 20° C. The reaction was stirred at 80° C. for 16 hours in sealed tube and was poured into water (10 mL) and extracted with ethyl acetate (10 mL*3). The combined organic phase was washed with brine (10 mL), dried with anhydrous Na₂SO₄, filtered and the filtrate was concentrated under reduced pressure. The residue was purified by prep-TLC (PE:EA=2:1, R_{f}=0.4) to give 61a. MS mass calculated for [M+H]⁺(C₂₆H₂₆F₃N₃O₅) requires m/z, 518.2, LCMS found m/z 518.0.

6-(4-((5-cyclopropyl-3-(2-(trifluoromethoxy)phenyl)isoxazol-4-yl)methoxy)piperidin-1-yl)nicotinohydrazide (61b). To a solution of methyl 6-(4-((5-cyclopropyl-3-(2-(trifluoromethoxy)phenyl)isoxazol-4-yl)methoxy)piperidin-1-yl)nicotinate (61a) (100 mg, 193.24 umol) in ethanol (4 mL) was added N₂H₄.H₂O (4.12 g, 80.65 mmol, 4.00 mL, 98% purity) at 20° C. in sealed tube, then the reaction was stirred at 20° C. for 6 hours. The reaction mixture was concentrated under reduced pressure to remove the solvent to give crude 61b.

5-(6-(4-((5-cyclopropyl-3-(2-(trifluoromethoxy)phenyl)isoxazol-4-yl)methoxy)piperidin-1-yl)pyridin-3-yl)-1,3,4-oxadiazol-2(3H)-one (Compound 61). To a solution of 6-(4-((5-cyclopropyl-3-(2-(trifluoromethoxy)phenyl)isoxazol-4-yl)methoxy)piperidin-1-yl)nicotinohydrazide (61b) (50 mg, 96.62 umol) in THF (10 mL) was added CDI (31.33 mg, 193.24 umol) and TEA (29.33 mg, 289.86 umol, 40.34 uL). The reaction mixture was stirred for 16 hours at 20° C. The mixture was concentrated under reduced pressure to remove the solvent. The residue was purified by prep-HPLC (column: Waters Xbridge BEH C18 100*25 mm*5 um; mobile phase: [water (10 mM NH₄HCO₃)-ACN]; B %: 35%-65%, 10 min) to give Compound 61. MS mass calculated for [M+H]⁺(C₂₆H₂₄F₃N₅O₅) requires m/z, 544.5, LCMS found m/z 544.2; ¹H NMR (400 MHz, CHLOROFORM-d) δ=8.80 (s, 1H), 8.2 (d, 1H), 7.81-7.80 (d, J=8, 1H), 7.58 (d, 1H), 7.56-7.49 (m, 1H), 7.40-7.27 (m, 2H), 7.65-7.63 (d, J=8 Hz, 2H), 4.42 (s, 2H), 3.87-3.83 (m, 2H), 3.54 (tt, J=3.7, 7.6 Hz, 1H), 3.36-3.2 (m, 2H), 2.14 (tt, J=5.3, 8.4 Hz, 1H), 1.84-1.78 (m, 2H), 1.55-1.51 (m, 2H), 1.25-1.23 (m, 2H), 1.13-1.10 (m, 2H).

### Example 62

### 4-(4-(4-((5-cyclopropyl-3-(2,6-dichlorophenyl)isoxazol-4-yl)methoxy)piperidin-1-yl)phenyl)-1,2,4-triazine-3, 5(2H,4H)-dione

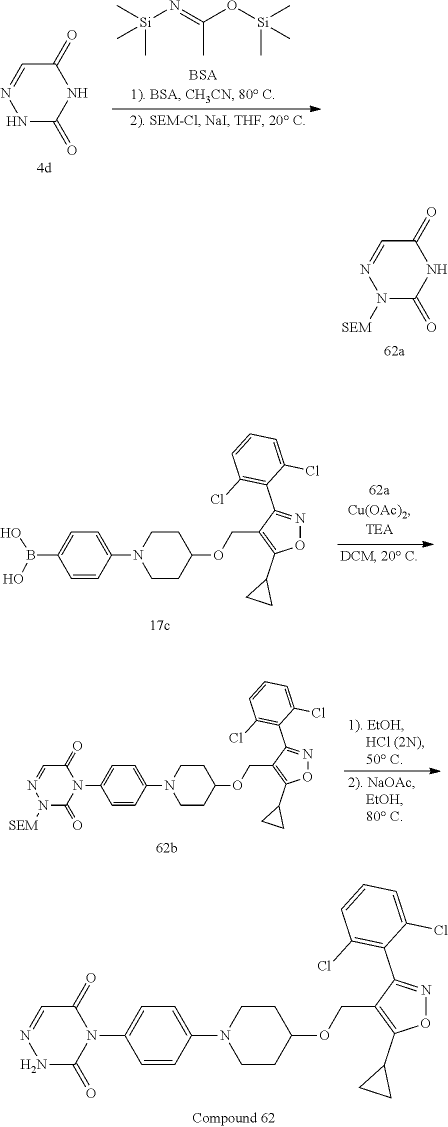

2-((2-(trimethylsilyl)ethoxy)methyl)-1,2,4-triazine-3,5(2H,4H)-dione (62a). To a solution of 1,2,4-triazine-3,5(2H,4H)-dione (4d) (100 mg, 884.37 umol, 1 eq) in acetonitrile (2 mL) was added (E)-trimethylsilyl N-(trimethylsilyl)acetimidate (359.82 mg, 1.77 mmol, 437.20 uL) at 20° C. The reaction was degassed and purged with N₂ three times, then heated to 80° C. and stirred under N₂ atmosphere. After 3 hours, the reaction mixture was cooled to 20° C., SEM-Cl (176.93 mg, 1.06 mmol, 187.83 uL) and NaI (26.51 mg, 176.87 umol) were added. The reaction was degassed and purged with N₂ 3 times and stirred for another 13 hours at this temperature. The reaction mixture was concentrated under reduced pressure to remove solvent. Water (25 mL) and ethyl acetate (25 mL) were added into the residue and the phases were separated. The aqueous phase was extracted with ethyl acetate (15 mL*4). The combined organic phase was washed with brine (20 mL*2), dried with anhydrous Na₂SO₄, filtered and the filtrate was concentrated under reduced pressure. The residue was purified by prep-TLC (SiO₂, dichloromethane:Methanol=10:1) to give 62a. ¹H NMR (400 MHz, CHLOROFORM-d) δ=8.67 (br s, 1H), 7.44 (s, 1H), 5.32 (s, 2H), 3.74-3.68 (m, 2H), 1.01-0.95 (m, 2H), 0.04-0.01 (m, 9H).

4-(4-(4-((5-cyclopropyl-3-(2,6-dichlorophenyl)isoxazol-4-yl)methoxy)piperidin-1-yl)phenyl)-2-((2-(trimethylsilyl)ethoxy)methyl)-1,2,4-triazine-3,5(2H,4H)-dione (62b). To a solution of (4-(4-((5-cyclopropyl-3-(2,6-dichlorophenyl)isoxazol-4-yl)methoxy)piperidin-1-yl)phenyl)boronic acid (17c) (40 mg, 82.10 umol) and 2-((2-(trimethylsilyl)ethoxy)methyl)-1,2,4-triazine-3,5(2H,4H)-dione (62a) (29.97 mg, 123.16 umol) in dichloromethane (4 mL) was added Cu(OAc)₂ (17.90 mg, 98.53 umol), TEA (16.62 mg, 164.21 umol, 22.86 uL) and Molecular sieve 4 A (40 mg) at 20° C. The mixture was stirred at 20° C. for 16 hours under O₂ ballon and was poured into H₂O (10 mL). The mixture was extracted with dichloromethane (15 mL*3). The combined organic layer was washed with brine (10 mL), dried over anhydrous Na₂SO₄, filtered and the filtrate was concentrated under reduced pressure to give a residue. The residue was purified by prep-TLC (SiO₂, dichloromethane:Methanol=20:1) to give 62b. MS mass calculated for [M+H]⁺(C₃₃H₃₉Cl₂N₅O₅Si) requires m/z, 684.2/686.2, LCMS found m/z, 684.3/686.2; ¹H NMR (400 MHz, CHLOROFORM-d) δ=7.56 (s, 1H), 7.40 (d, J=1.0 Hz, 1H), 7.38 (s, 1H), 7.33-7.29 (m, 1H), 7.07 (d, J=8.9 Hz, 2H), 6.97-6.90 (m, 2H), 5.36 (s, 2H), 4.35 (s, 2H), 3.78-3.71 (m, 2H), 3.42 (qd, J=3.9, 7.7 Hz, 1H), 3.34-3.26 (m, 2H), 2.97-2.89 (m, 2H), 2.21-2.12 (m, 1H), 1.85-1.75 (m, 2H), 1.59 (br d, J=3.3 Hz, 1H), 1.55-1.50 (m, 1H), 1.30-1.25 (m, 2H), 1.17-1.11 (m, 2H), 1.03-0.96 (m, 2H), 0.06-−0.01 (m, 9H).

4-(4-(4-((5-cyclopropyl-3-(2,6-dichlorophenyl)isoxazol-4-yl)methoxy)piperidin-1-yl)phenyl)-1,2,4-triazine-3,5(2H,4H)-dione (Compound 62). To a solution of 4-(4-(4-((5-cyclopropyl-3-(2,6-dichlorophenyl)isoxazol-4-yl)methoxy)piperidin-1-yl)phenyl)-2-((2-(trimethylsilyl)ethoxy)methyl)-1,2,4-triazine-3,5(2H,4H)-dione (62b) (10 mg, 14.61 umol) in ethanol (0.2 mL) was added aqueous HCl (2 M, 0.4 mL) at 20° C., and the mixture was heated to 50° C. for 16 hours. The reaction mixture was concentrated under reduced pressure. Ethanol (0.5 mL) and NaOAc (9.59 mg, 116.84 umol) were added to the mixture and the mixture was heated to 80° C. and stirred for 16 hours. The reaction mixture was poured into H₂O (5 mL) and was extracted with ethyl acetate (10 mL*2). The combined organic layer was washed with brine (5 mL), dried over anhydrous Na₂SO₄, filtered and the filtrate was concentrated under reduced pressure to give a residue. The residue was purified by prep-HPLC (column: Phenomenex Gemini-NX C18 75*30 mm*3 um; mobile phase: [water (10 mM NH₄HCO₃)-ACN]; B %: 40%-70%, 8 min) to give Compound 62. MS mass calculated for [M+H]⁺(C₂₇H₂₅Cl₂N₅O₄) requires m/z, 554.1/556.1, LCMS found m/z, 554.2/556.1; ¹H NMR (400 MHz, CHLOROFORM-d) δ=9.31 (br s, 1H), 7.52 (s, 1H), 7.42-7.36 (m, 2H), 7.35-7.28 (m, 1H), 7.09 (d, J=8.8 Hz, 2H), 6.94 (d, J=9.0 Hz, 2H), 4.35 (s, 2H), 3.43 (td, J=3.9, 7.7 Hz, 1H), 3.37-3.28 (m, 2H), 2.93 (ddd, J=3.1, 8.8, 12.3 Hz, 2H), 2.21-2.12 (m, 1H), 1.84-1.75 (m, 2H), 1.57-1.50 (m, 2H), 1.32-1.25 (m, 2H), 1.18-1.10 (m, 2H).

### Example 63

### 3-(6-(4-((5-cyclopropyl-3-(2,6-difluorophenyl)isoxazol-4-yl)methoxy)-3,3-difluoropiperidin-1-yl)pyridin-3-yl)-1,2,4-oxadiazol-5(4H)-one

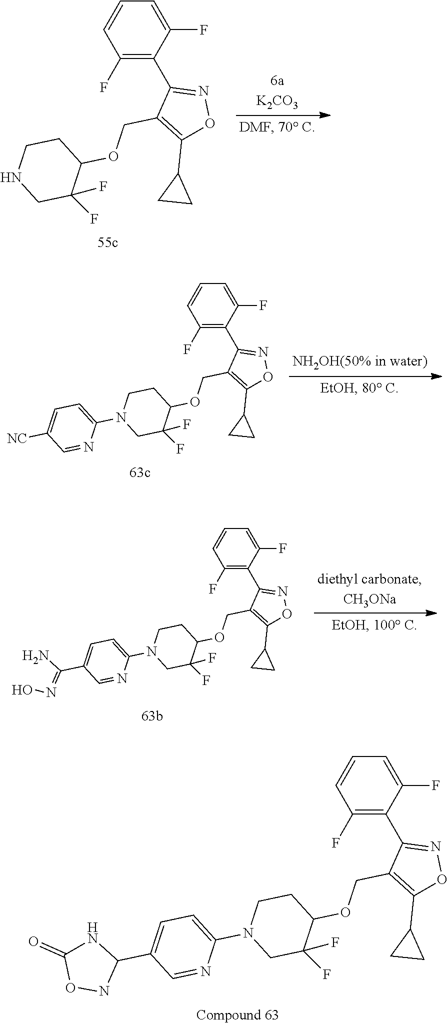 [SMILES: Fc1cccc(F)c1-c1noc(C2CC2)c1COC1CCNCC1(F)F ; N#Cc1ccc(N2CCC(OCc3c(-c4c(F)cccc4F)noc3C3CC3)C(F)(F)C2)nc1 ; N/C(=N\O)c1ccc(N2CCC(OCc3c(-c4c(F)cccc4F)noc3C3CC3)C(F)(F)C2)nc1 ; O=C1NC(c2ccc(N3CCC(OCc4c(-c5c(F)cccc5F)noc4C4CC4)C(F)(F)C3)nc2)NO1]

6-(4-((5-cyclopropyl-3-(2,6-difluorophenyl)isoxazol-4-yl)methoxy)-3,3-difluoropiperidin-1-yl)nicotinonitrile (63a). To a solution of 5-cyclopropyl-3-(2,6-difluorophenyl)-4-(((3,3-difluoropiperidin-4-yl)oxy)methyl)isoxazole (55c) (100 mg, 270.02 umol) in DMF (5 mL) was added K₂CO₃ (149.27 mg, 1.08 mmol) and 6-fluoronicotinonitrile (6a) (98.91 mg, 810.07 umol) at 20° C. and the mixture was heated to 70° C. for 16 hours. The reaction mixture was poured into H₂O (15 mL) and the resulting mixture was extracted with ethyl acetate (20 mL*3). The combined organic layer was washed with brine (15 mL), dried over anhydrous Na₂SO₄, filtered and the filtrate was concentrated under reduced pressure to give a residue. The residue was purified by prep-TLC (Petroleum ether:ethyl acetate=1:1) to give 63a. MS mass calculated for [M+H]⁺(C₂₄H₂₀F₄N₄O₂) requires m/z, 473.2, LCMS found m/z, 473.1.

(Z)-6-(4-((5-cyclopropyl-3-(2,6-difluorophenyl)isoxazol-4-yl)methoxy)-3,3-difluoropiperidin-1-yl)-N′-hydroxynicotinimidamide (63b). To a solution of 6-(4-((5-cyclopropyl-3-(2,6-difluorophenyl)isoxazol-4-yl)methoxy)-3,3-difluoropiperidin-1-yl)nicotinonitrile (63a) (80 mg, 169.34 umol) in ethanol (4 mL) was added hydroxylamine (0.8 mL, 50% in water) at 20° C. and the mixture was heated to 80° C. for 2 hours. The reaction mixture was poured into H₂O (10 mL) and extracted with ethyl acetate (20 mL*2). The combined organic layer was washed with brine (10 mL), dried over anhydrous Na₂SO₄, filtered and the filtrate was concentrated under reduced pressure to give a residue. The residue was purified by prep-TLC (SiO₂, dichloromethane:methanol=10:1) to give 63b. MS mass calculated for [M+H]⁺(C₂₄H₂₃F₄N₅O₃) requires m/z, 506.2, LCMS found m/z, 506.2; ¹H NMR (400 MHz, CHLOROFORM-d) δ=8.39 (d, J=2.1 Hz, 1H), 7.72 (dd, J=2.4, 8.9 Hz, 1H), 7.51-7.38 (m, 1H), 7.10-6.97 (m, 2H), 6.62 (d, J=9.0 Hz, 1H), 4.80 (br s, 2H), 4.69 (d, J=12.0 Hz, 1H), 4.49 (d, J=12.1 Hz, 1H), 4.16-4.02 (m, 1H), 3.74-3.53 (m, 3H), 3.34-3.24 (m, 1H), 2.15 (tt, J=5.1, 8.4 Hz, 1H), 1.87-1.76 (m, 1H), 1.66 (br dd, J=4.3, 9.5 Hz, 1H), 1.29-1.23 (m, 2H), 1.18-1.10 (m, 2H).

3-(6-(4-((5-cyclopropyl-3-(2,6-difluorophenyl)isoxazol-4-yl)methoxy)-3,3-difluoropiperidin-1-yl)pyridin-3-yl)-1,2,4-oxadiazol-5(4H)-one (Compound 63). To a solution of (Z)-6-(4-((5-cyclopropyl-3-(2,6-difluorophenyl)isoxazol-4-yl)methoxy)-3,3-difluoropiperidin-1-yl)-N′-hydroxynicotinimidamide (63b) (60 mg, 118.70 umol) in ethanol (3 mL) was added diethyl carbonate (585.00 mg, 4.95 mmol, 0.6 mL) and CH₃ONa (106.87 mg, 593.91 umol, 30% in MeOH) at 20° C. The reaction mixture was heated to 100° C. for 0.5 hour and was poured into H₂O (10 mL) and was extracted with ethyl acetate (20 mL*2). The combined organic layer was washed with brine (10 mL), dried over anhydrous Na₂SO₄, filtered and the filtrate was concentrated under reduced pressure to give a residue. The residue was purified by prep-HPLC (column: Phenomenex Gemini-NX 150*30 mm*5 um; mobile phase: [water (10 Mm NH₄HCO₃)-ACN]; B %: 15%-45%, 8 min) to give Compound 63. MS mass calculated for [M+H]⁺(C₂₅H₂₁F₄N₅O₄) requires m/z, 532.2, LCMS found m/z, 532.2; ¹H NMR (400 MHz, CHLOROFORM-d) δ=8.50 (s, 1H), 7.82 (br d, J=7.5 Hz, 1H), 7.50-7.41 (m, 1H), 7.04 (t, J=7.8 Hz, 2H), 6.71 (br d, J=9.2 Hz, 1H), 4.70 (d, J=12.0 Hz, 1H), 4.50 (d, J=12.0 Hz, 1H), 4.29-4.18 (m, 1H), 3.84 (br d, J=14.1 Hz, 1H), 3.65 (br s, 1H), 3.63-3.53 (m, 1H), 3.31 (br t, J=11.4 Hz, 1H), 2.19-2.10 (m, 1H), 1.82 (br s, 1H), 1.67 (br d, J=14.5 Hz, 1H), 1.26 (br s, 2H), 1.15 (br dd, J=2.8, 8.1 Hz, 2H).

### Example 64

### 3-(6-(4-((5-cyclopropyl-3-(2-(trifluoromethoxy)phenyl)isoxazol-4-yl)methoxy)-3,3-difluoropiperidin-1-yl)pyridin-3-yl)-1,2,4-oxadiazol-5(4H)-one

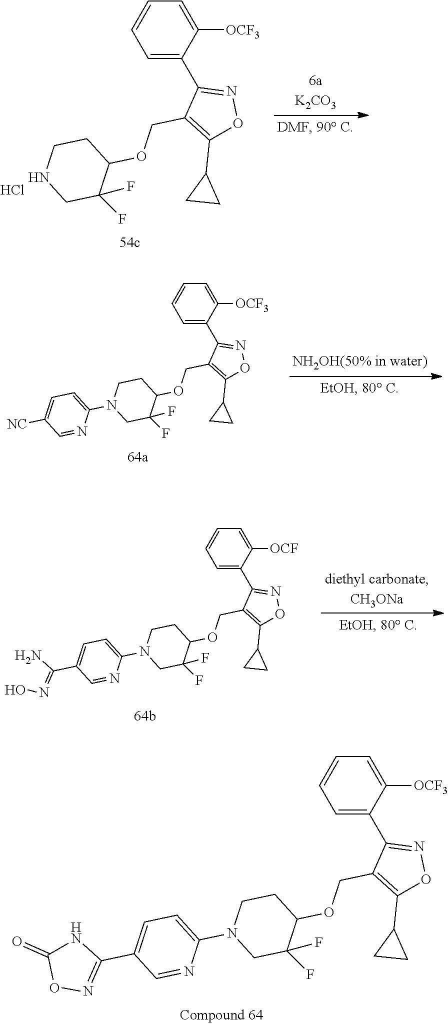

6-(4-((5-cyclopropyl-3-(2-(trifluoromethoxy)phenyl)isoxazol-4-yl)methoxy)-3,3-difluoropiperidin-1-yl)nicotinonitrile (64a). To a mixture of 5-cyclopropyl-4-(((3,3-difluoropiperidin-4-yl)oxy)methyl)-3-(2-(trifluoromethoxy)phenyl)isoxazole hydrochloride (54c) (40 mg, 87.95 umol) and 6-fluoronicotinonitrile (6a) (21.48 mg, 175.89 umol) in DMF (1 mL) was added K₂CO₃ (36.46 mg, 263.84 umol) at 15° C. and the mixture was heated to 90° C. and stirred for 16 hours. Water (10 mL) was added to the mixture and the resulting mixture was extracted with ethyl acetate (10 mL*3). The combined organic phase was washed with brine (10 mL), dried over anhydrous Na₂SO₄ and the filtrate was concentrated under reduced pressure. The residue was purified by prep-TLC to give 64a. ¹H NMR (400 MHz, CHLOROFORM-d) δ=8.40 (d, J=1.8 Hz, 1H), 7.63 (dd, J=2.3, 9.0 Hz, 1H), 7.58-7.50 (m, 2H), 7.43-7.37 (m, 2H), 6.66-6.58 (m, 1H), 4.70 (d, J=11.7 Hz, 1H), 4.50 (d, J=11.7 Hz, 1H), 4.22 (td, J=8.8, 14.3 Hz, 1H), 3.82 (br d, J=14.1 Hz, 1H), 3.69-3.53 (m, 2H), 3.33-3.22 (m, 1H), 2.13 (tt, J=5.1, 8.4 Hz, 1H), 1.86-1.74 (m, 1H), 1.71-1.61 (m, 1H), 1.28-1.26 (m, 1H), 1.26 (br s, 1H), 1.25-1.22 (m, 1H), 1.18-1.11 (m, 2H).

(Z)-6-(4-((5-cyclopropyl-3-(2-(trifluoromethoxy)phenyl)isoxazol-4-yl)methoxy)-3,3-difluoropiperidin-1-yl)-N′-hydroxynicotinimidamide (64b). To a solution of 6-(4-((5-cyclopropyl-3-(2-(trifluoromethoxy)phenyl)isoxazol-4-yl)methoxy)-3,3-difluoropiperidin-1-yl)nicotinonitrile (64a) (37 mg, 71.09 umol) in ethanol (2 mL) was added hydroxylamine (0.4 mL, 50% in water) at 15° C. and the mixture was heated to 80° C. and stirred for 0.5 hour. Water (10 mL) was added to the mixture and the resulting mixture was extracted with ethyl acetate (10 mL*3). The combined organic phase was washed with brine (10 mL), dried over anhydrous Na₂SO₄, filtered and the filtrate was concentrated under reduced pressure to give 64b. MS mass calculated for [M+H]⁺(C₂₅H₂₄F₅N₅O₄) requires m/z, 554.2, LCMS found m/z, 554.1.

### 3-(6-(4-((5-cyclopropyl-3-(2-(trifluoromethoxy)phenyl)isoxazol-4-yl)methoxy)-3,3-difluoropiperidin-1-yl)pyridin-3-yl)-1,2,4-oxadiazol-5(4H)-one (Compound 64)

To a solution of (Z)-6-(4-((5-cyclopropyl-3-(2-(trifluoromethoxy)phenyl)isoxazol-4-yl)methoxy)-3,3-difluoropiperidin-1-yl)-N′-hydroxynicotinimidamide (64b) (20 mg, 36.13 umol) in ethanol (3 mL) was added diethyl carbonate (195.00 mg, 1.65 mmol, 0.2 mL) and CH₃ONa (32.54 mg, 180.67 umol, 30% in MeOH) at 15° C. and the mixture was heated to 100° C. and stirred for 0.5 hour. Water (10 mL) was added to the mixture and the resulting mixture was extracted by ethyl acetate (10 mL*3). The combined organic phase was washed with brine, dried with anhydrous Na₂SO₄, filtered and the filtrate was concentrated under reduced pressure. The residue was purified by prep-TLC to give Compound 64. MS mass calculated for [M+H]⁺(C₂₆H₂₂F₅N₅O₅) requires m/z, 580.2, LCMS found m/z, 580.2; ¹H NMR (400 MHz, CHLOROFORM-d) δ=8.51 (d, J=2.2 Hz, 1H), 7.82 (dd, J=2.4, 9.1 Hz, 1H), 7.60-7.50 (m, 2H), 7.45-7.36 (m, 2H), 6.77-6.64 (m, 1H), 4.70 (d, J=11.7 Hz, 1H), 4.51 (d, J=11.9 Hz, 1H), 4.29-4.14 (m, 1H), 3.82 (br d, J=13.8 Hz, 1H), 3.72-3.53 (m, 2H), 3.31 (br t, J=10.5 Hz, 1H), 2.14 (tt, J=5.1, 8.4 Hz, 1H), 1.89-1.75 (m, 1H), 1.72-1.62 (m, 1H), 1.29-1.22 (m, 2H), 1.18-1.11 (m, 2H).

### Example 65

### 5-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)-1,3,4-oxadiazol-2(3H)-one

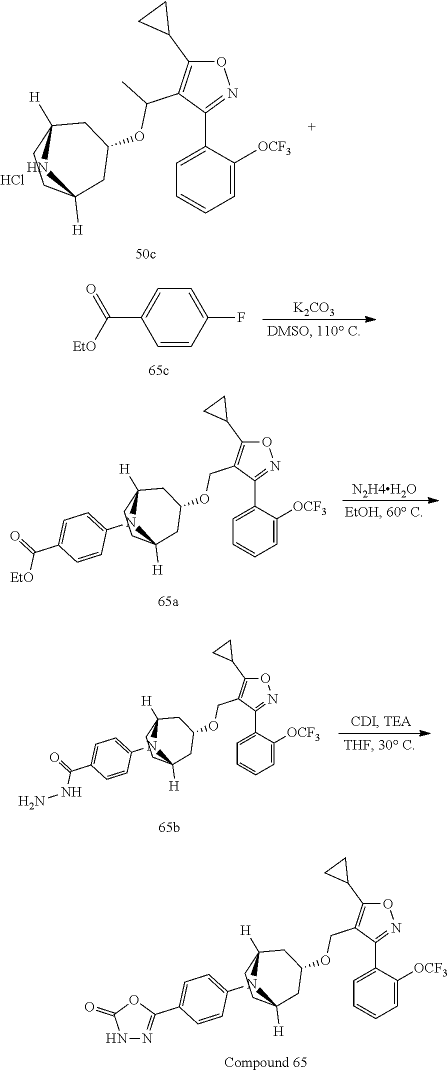

Ethyl 4-((1R,3R,5S)-3-((5-cyclopropyl-3-(2-(trifluoromethoxy)phenyl)isoxazol-4-yl)methoxy)-8-azabicyclo[3.2.1]octan-8-yl)benzoate (65a). To a solution of 4-(((1R,3R,5S)-8-azabicyclo[3.2.1]octan-3-yloxy)methyl)-5-cyclopropyl-3-(2-(trifluoromethoxy)phenyl)isoxazole hydrochloride (50c) (100 mg, 224.78 umol) in DMSO (5 mL) was added K₂CO₃ (186.40 mg, 1.35 mmol) and ethyl 4-fluorobenzoate (65c) (189.00 mg, 1.12 mmol, 165.79 uL) in a sealed tube. The resulting mixture was heated to 110° C. and stirred for 96 hours. The mixture was cooled to room temperature and diluted with water (5 mL), extracted with ethyl acetate (5 mL*4). The combined organic phase was washed with brine (20 mL*2), dried over anhydrous Na₂SO₄, filtered and the filtrate was concentrated. The residue was purified by prep-TLC (SiO₂, petroleum ether:ethyl acetate=3:1) to give 65a. MS mass calculated for [M+H]⁺(C₃₀H₃₁F₃N₂O₅) requires m/z, 557.2/558.2, LCMS found m/z, 557.3/558.3; ¹H NMR (400 MHz, CHLOROFORM-d) δ=7.89 (d, J=8.7 Hz, 2H), 7.58-7.49 (m, 2H), 7.39 (t, J=7.3 Hz, 2H), 6.65 (d, J=8.8 Hz, 2H), 4.35-4.28 (m, 4H), 4.14 (br s, 2H), 3.42 (t, J=4.7 Hz, 1H), 2.16-2.08 (m, 1H), 1.99-1.85 (m, 6H), 1.60 (s, 1H), 1.57 (s, 1H), 1.36 (t, J=7.2 Hz, 3H), 1.25-1.20 (m, 2H), 1.14-1.09 (m, 2H).

4-((1R,3R,5S)-3-((5-cyclopropyl-3-(2-(trifluoromethoxy)phenyl)isoxazol-4-yl)methoxy)-8-azabicyclo[3.2.1]octan-8-yl)benzohydrazide (65b). To a solution of ethyl 4-((1R,3R,5S)-3-((5-cyclopropyl-3-(2-(trifluoromethoxy)phenyl)isoxazol-4-yl) methoxy)-8-azabicyclo[3.2.1]octan-8-yl)benzoate (65a) (40 mg, 71.87 umol) in ethanol (1.5 mL) was added hydrazine hydrate (2.06 g, 41.15 mmol, 2 mL) at 20° C. The reaction was stirred at 55° C. for 36 hours and was concentrated under reduced pressure to give crude 65b. MS mass calculated for [M+H]⁺(C₂₈H₂₉F₃N₄O₄) requires m/z, 543.2/544.2, LCMS found m/z 543.3/544.2.

5-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)-1,3,4-oxadiazol-2(3H)-one (Compound 65). To a mixture of 4-((1R,3R,5S)-3-((5-cyclopropyl-3-(2-(trifluoromethoxy)phenyl)isoxazol-4-yl)methoxy)-8-azabicyclo[3.2.1]octan-8-yl)benzohydrazide (65b) (40 mg, 73.73 umol) in THF (2 mL) was added CDI (35.86 mg, 221.18 umol) and TEA (29.84 mg, 294.90 umol, 41.05 uL) at 20° C. The reaction was stirred at 30° C. for 16 hours. Water (5 mL) and ethyl acetate (5 mL) were added into the residue and the phases were separated. The aqueous phase was extracted with ethyl acetate (5 mL*4). The combined organic phase was washed with brine (10 mL*2), dried with anhydrous Na₂SO₄, filtered and the filtrate was concentrated under reduced pressure and purified by prep-HPLC (neutral condition: column: Waters Xbridge BEH C18 100*25 mm*5 um; mobile phase: [water(10 mM NH₄HCO₃)-ACN]; B %: 50%-80%, 10 min) and lyophilized to give Compound 65. MS mass calculated for [M+H]⁺(C₂₉H₂₇F₃N₄O₅) requires m/z, 569.2/570.2, LCMS found m/z 569.2/570.2; ¹H NMR (400 MHz, CHLOROFORM-d) δ=8.37 (s, 1H), 7.67 (d, J=9.0 Hz, 2H), 7.58-7.49 (m, 2H), 7.39 (t, J=7.1 Hz, 2H), 6.71 (d, J=8.8 Hz, 2H), 4.31 (s, 2H), 4.13 (br s, 2H), 3.44 (t, J=4.6 Hz, 1H), 2.12 (tt, J=5.1, 8.5 Hz, 1H), 2.01-1.85 (m, 6H), 1.61 (s, 2H), 1.27-1.21 (m, 2H), 1.15-1.09 (m, 2H).

### Example 66

### 3-(6-((1R,3r,5S)-3-((5-cyclopropyl-3-(2-(trifluoromethoxy)phenyl)isoxazol-4-yl)methoxy)-8-azabicyclo[3.2.1]octan-8-yl)pyridin-3-yl)-1,2,4-oxadiazol-5(4H)-one

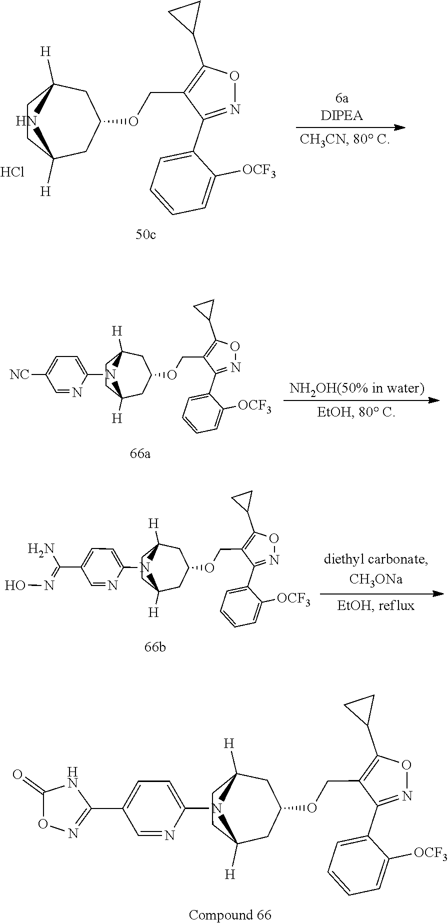 [SMILES: Cl ; FC(F)(F)Oc1ccccc1-c1noc(C2CC2)c1CO[C@H]1C[C@H]2CC[C@@H](C1)N2 ; N#Cc1ccc(N2[C@@H]3CC[C@H]2C[C@@H](OCc2c(-c4ccccc4OC(F)(F)F)noc2C2CC2)C3)nc1 ; "NH2OH(50% in water)" ; N/C(=N\O)c1ccc(N2[C@@H]3CC[C@H]2C[C@@H](OCc2c(-c4ccccc4OC(F)(F)F)noc2C2CC2)C3)nc1 ; O=c1[nH]c(-c2ccc(N3[C@@H]4CC[C@H]3C[C@@H](OCc3c(-c5ccccc5OC(F)(F)F)noc3C3CC3)C4)nc2)no1]

6-((1R,3R,5S)-3-((5-cyclopropyl-3-(2-(trifluoromethoxy)phenyl)isoxazol-4-yl)methoxy)-8-azabicyclo[3.2.1]octan-8-yl)nicotinonitrile (66a). To a solution of 4-(((1R,3R,5S)-8-azabicyclo[3.2.1]octan-3-yloxy)methyl)-5-cyclopropyl-3-(2-(trifluoromethoxy)phenyl)isoxazole hydrochloride (50c) (100 mg, 224.78 umol) in CH₃CN (1 mL) was added 6-fluoronicotinonitrile (6a) (30.19 mg, 247.26 umol) and DIPEA (87.15 mg, 674.35 umol, 117.46 uL). The reaction mixture was heated to 90° C. and stirred for 18 hours and was concentrated under reduced pressure. The residue was diluted with water (5 mL) and extracted with ethyl acetate (10 mL*2). The combined organic phase was washed with brine (10 mL), dried over anhydrous Na₂SO₄, filtered and the filtrate was concentrated under reduced pressure. The residue was purified by prep-TLC (SiO₂, petroleum ether:ethyl acetate=3:1) to give 66a. MS mass calculated for [M+H]⁺(C₂₇H₂₅F₃N₄O₃) requires m/z, 511.2, LCMS found m/z, 511.1; ¹H NMR (400 MHz, CHLOROFORM-d) δ=8.38 (d, J=2.4 Hz, 1H), 7.59-7.49 (m, 3H), 7.44-7.34 (m, 2H), 6.42 (d, J=8.3 Hz, 1H), 4.62-4.35 (m, 2H), 4.33 (s, 2H), 3.49 (t, J=4.6 Hz, 1H), 2.12 (tt, J=5.3, 8.4 Hz, 1H), 2.01-1.92 (m, 2H), 1.91-1.81 (m, 4H), 1.69 (m, 2H), 1.27-1.20 (m, 2H), 1.16-1.06 (m, 2H).

(Z)-6-((1R,3R,5S)-3-((5-cyclopropyl-3-(2-(trifluoromethoxy)phenyl)isoxazol-4-yl)methoxy)-8-azabicyclo[3.2.1]octan-8-yl)-N′-hydroxynicotinimidamide (66b). To a solution of 6-((1R,3R,5S)-3-((5-cyclopropyl-3-(2-(trifluoromethoxy)phenyl)isoxazol-4-yl)methoxy)-8-azabicyclo[3.2.1]octan-8-yl)nicotinonitrile (66a) (70 mg, 137.12 umol) in ethanol (3 mL) was added hydroxylamine (1.5 mL, 50% in water). The reaction mixture was heated to 80° C. and stirred for 2 hours and was concentrated under reduced pressure. The residue was diluted with water (10 mL) and extracted with dichloromethane (20 mL*3). The combined organic phase was washed with brine (10 mL), dried over anhydrous Na₂SO₄, filtered and the filtrate was concentrated to give 66b. MS mass calculated for [M+H]⁺(C₂₇H₂₈F₃N₅O₄) requires m/z, 544.2, LCMS found m/z, 544.2; ¹H NMR (400 MHz, CHLOROFORM-d) δ=8.38 (d, J=2.0 Hz, 1H), 7.68 (dd, J=2.4, 8.8 Hz, 1H), 7.59-7.48 (m, 2H), 7.43-7.35 (m, 2H), 6.47 (d, J=8.8 Hz, 1H), 4.76 (br s, 2H), 4.44-4.34 (m, 2H), 4.32 (s, 2H), 3.46 (br t, J=4.6 Hz, 1H), 2.13 (tt, J=5.1, 8.4 Hz, 1H), 1.99-1.93 (m, 2H), 1.92-1.85 (m, 4H), 1.65 (br d, J=14.2 Hz, 2H), 1.28-1.19 (m, 2H), 1.16-1.04 (m, 2H).

3-(6-((1R,3R,5S)-3-((5-cyclopropyl-3-(2-(trifluoromethoxy)phenyl)isoxazol-4-yl)methoxy)-8-azabicyclo[3.2.1]octan-8-yl)pyridin-3-yl)-1,2,4-oxadiazol-5(4H)-one (Compound 66). To a solution of (Z)-6-((1R,3R,5S)-3-((5-cyclopropyl-3-(2-(trifluoromethoxy)phenyl)isoxazol-4-yl)methoxy)-8-azabicyclo[3.2.1]octan-8-yl)-N′-hydroxynicotinimidamide (66b) (60 mg, 110.39 umol) in ethanol (1 mL) was added diethyl carbonate (782.41 mg, 6.62 mmol, 802.47 uL) and CH₃ONa (119.26 mg, 662.33 umol, 30% in MeOH). The mixture was heated to 100° C. and stirred for 1 hour and concentrated under reduced pressure. The residue was diluted with H₂O (10 mL) and extracted with dichloromethane (10 mL*3). The combined organic phase was washed with brine (10 mL), dried over anhydrous Na₂SO₄, filtered and the filtrate was concentrated under reduced pressure. The residue was purified by prep-HPLC (neutral condition; column: Phenomenex Gemini-NX 150*30 mm*5 um; mobile phase: [water (10 mM NH₄HCO₃)-ACN]; B %: 15%-45%, 8 min) to give Compound 66. MS mass calculated for [M+H]⁺(C₂₈H₂₆F₃N₅O₅) requires m/z, 570.2, LCMS found m/z, 570.2; ¹H NMR (400 MHz, CHLOROFORM-d) δ=8.45 (br s, 1H), 7.76 (dd, J=2.4, 8.8 Hz, 1H), 7.60-7.47 (m, 2H), 7.45-7.33 (m, 2H), 6.53 (br d, J=8.8 Hz, 1H), 4.41 (br s, 2H), 4.33 (s, 2H), 3.49 (br s, 1H), 2.17-2.05 (m, 1H), 2.04-1.81 (m, 6H), 1.70 (br d, J=14.2 Hz, 2H), 1.27-1.18 (m, 2H), 1.16-1.07 (m, 2H).

### Example 67

### 6-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)-1,2,4-triazine-3,5(2H,4H)-dione

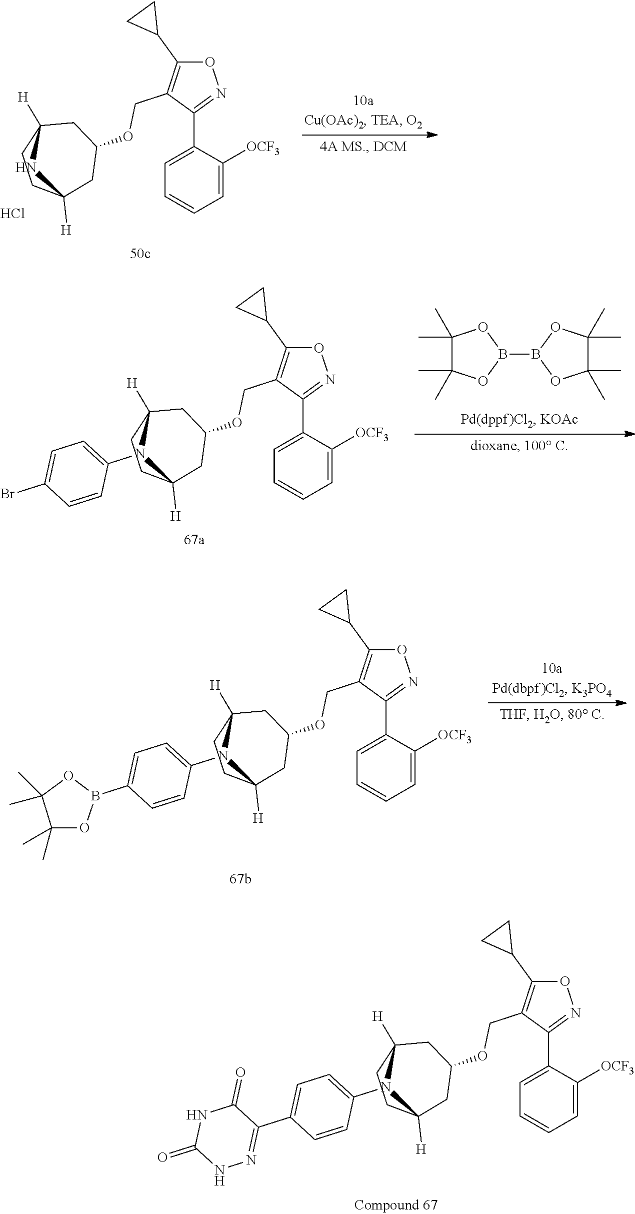

4-((((1R,3R,5S)-8-(4-bromophenyl)-8-azabicyclo[3.2.1]octan-3-yl)oxy)methyl)-5-cyclopropyl-3-(2-(trifluoromethoxy)phenyl)isoxazole (67a). To a mixture of 4-(((1R,3R,5S)-8-azabicyclo[3.2.1]octan-3-yloxy)methyl)-5-cyclopropyl-3-(2-(trifluoromethoxy)phenyl)isoxazole hydrochloride (50c) (80 mg, 195.88 umol) and (4-bromophenyl)boronic acid (10a) (59.01 mg, 293.82 umol) in dichloromethane (5 mL) was added Cu(OAc)₂ (42.69 mg, 235.06 umol), TEA (39.64 mg, 391.76 umol, 54.53 uL) and Molecular sieve 4 A (10 mg) at 20° C. under N₂. The suspension was degassed and purged with O₂ several times. The mixture was stirred at 20° C. for 16 hours and was filtered. The filtrate was concentrated under reduced pressure. The residue was purified by prep-TLC to give 67a. MS mass calculated for [M+H]⁺(C₂₇H₂₆BrF₃N₂O₃) requires m/z, 563.1/565.1, LCMS found m/z, 563.2/565.1.

5-cyclopropyl-4-((((1R,3R,5S)-8-(4-(4,4,5,5-tetramethyl-1,3,2-dioxaborolan-2-yl)phenyl)-8-azabicyclo[3.2.1]octan-3-yl)oxy)methyl)-3-(2-(trifluoromethoxy)phenyl)isoxazole (67b). To a mixture of 4-((((1R,3R,5S)-8-(4-bromophenyl)-8-azabicyclo[3.2.1]octan-3-yl)oxy)methyl)-5-cyclopropyl-3-(2-(trifluoromethoxy)phenyl)isoxazole (67a) (50 mg, 88.75 umol) and 4,4,5,5-tetramethyl-2-(4,4,5,5-tetramethyl-1,3,2-dioxaborolan-2-yl)-1,3,2-dioxaborolane (67.61 mg, 266.24 umol) in 1, 4-dioxane (5 mL) was added Pd(dppf)Cl₂ (6.49 mg, 8.87 umol) and KOAc (17.42 mg, 177.49 umol) at 20° C. under N₂. The mixture was stirred at 100° C. for 16 hours and was poured into ice-water (20 mL). The mixture was extracted with ethyl acetate (20 mL). The organic phase was washed with brine (10 mL*2), dried with anhydrous Na₂SO₄, filtered and the filtrate was concentrated under reduced pressure. The residue was purified by prep-TLC to give 67b. MS mass calculated for [M+H]⁺(C₃₃H₃₈BF₃N₂O₅) requires m/z, 611.3/612.3, LCMS found m/z, 611.3/612.3; ¹H NMR (400 MHz, CHLOROFORM-d) δ=7.68 (d, J=8.7 Hz, 2H), 7.58-7.49 (m, 2H), 7.43-7.37 (m, 2H), 6.69 (d, J=8.7 Hz, 2H), 4.30 (s, 2H), 4.13 (br s, 2H), 3.39 (t, J=4.8 Hz, 1H), 2.18-2.09 (m, 1H), 2.03-1.85 (m, 6H), 1.34 (s, 12H), 1.25-1.21 (m, 2H), 1.15-1.09 (m, 2H).

6-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)-1,2,4-triazine-3,5(2H,4H)-dione (Compound 67). To a mixture of 5-cyclopropyl-4-((((1R,3R,5S)-8-(4-(4,4,5,5-tetramethyl-1,3,2-dioxaborolan-2-yl)phenyl)-8-azabicyclo[3.2.1]octan-3-yl)oxy)methyl)-3-(2-(trifluoromethoxy)phenyl)isoxazole (67b) (30 mg, 49.14 umol) and 6-bromo-1,2,4-triazine-3,5(2H,4H)-dione (10d) (28.30 mg, 147.43 umol) in THE (2 mL) and H₂O (0.5 mL) was added K₃PO₄ (20.86 mg, 98.28 umol) and ditertbutyl(cyclopentyl)phosphane; dichloropalladium:iron (3.20 mg, 4.91 umol) at 20° C. under N₂. The suspension was degassed and purged with N₂ three times then heated to 80° C. and stirred for 16 hours. The reaction mixture was poured into water (10 mL). The mixture was extracted with ethyl acetate (10 mL*2). The combined organic phase was washed with brine (20 mL), dried with anhydrous Na₂SO₄, filtered and concentrated under reduced pressure. The residue was purified by prep-HPLC (neutral condition) and lyophilzied to give Compound 67. MS mass calculated for [M+H]⁺(MS mass calculated for [M+H]⁺(C₃₀H₂₈F₃N₅O₅) requires m/z, 596.20, LCMS found m/z, 596.2; ¹H NMR (400 MHz, CHLOROFORM-d) δ 9.21 (s, 1H), 8.47 (br s, 1H), 7.94 (d, J=9.0 Hz, 2H), 7.63-7.49 (m, 2H), 7.41 (t, J=7.1 Hz, 2H), 6.73 (d, J=9.0 Hz, 2H), 4.32 (s, 2H), 4.14 (br s, 2H), 3.43 (br s, 1H), 2.21-2.09 (m, 1H), 2.03-1.84 (m, 6H), 1.61 (br s, 2H), 1.29-1.21 (m, 2H), 1.17-1.06 (m, 2H).

### Example 68

### 5-(6-((1R,3R,5S)-3-((5-cyclopropyl-3-(2-(trifluoromethoxy)phenyl)isoxazol-4-yl)methoxy)-8-azabicyclo[3.2.1]octan-8-yl)pyridin-3-yl)-1,3,4-oxadiazol-2(3H)-one

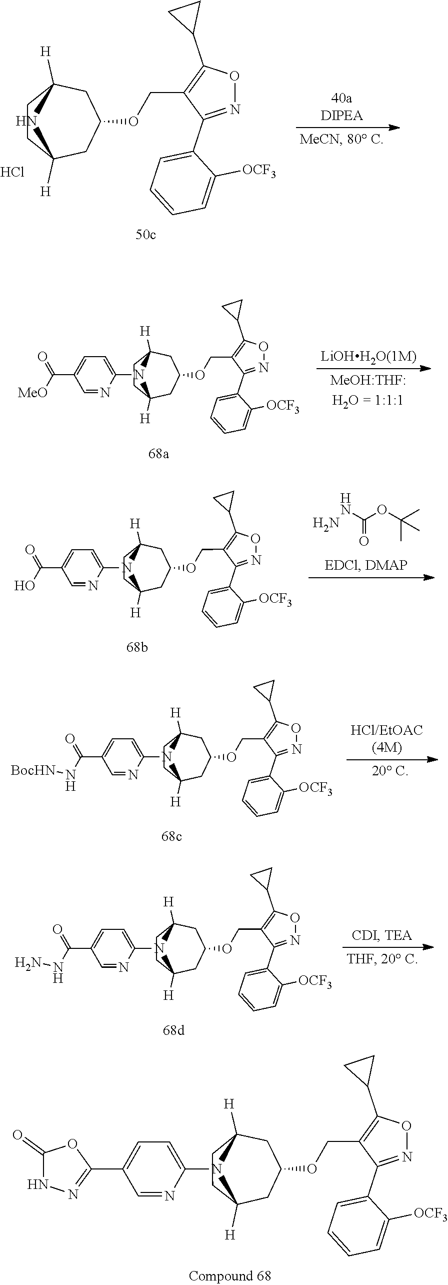

Methyl 6-((1R,3R,5S)-3-((5-cyclopropyl-3-(2-(trifluoromethoxy)phenyl)isoxazol-4-yl)methoxy)-8-azabicyclo[3.2.1]octan-8-yl)nicotinate (68a). To a mixture 4-((((1R,3R,5S)-8-azabicyclo[3.2.1]octan-3-yl)oxy)methyl)-5-cyclopropyl-3-(2-(trifluoromethoxy)phenyl)isoxazole hydrochloride (50c) (100 mg, 245.44 umol) and methyl 6-fluoronicotinate (40a) (45.69 mg, 294.53 umol) in acetonitrile (3 mL) was added DIPEA (158.61 mg, 1.23 mmol, 213.75 uL) at 20° C. under N₂. The mixture was stirred at 80° C. for 16 hours and was concentrated under reduced pressure. The residue was purified by prep-TLC to give 68a. MS mass calculated for [M+H]⁺(C₂₈H₂₈F₃N₃O₅) requires m/z, 544.2/545.2, LCMS found m/z, 544.2/545.2.

6-((1R,3R,5S)-3-((5-cyclopropyl-3-(2-(trifluoromethoxy)phenyl)isoxazol-4-yl)methoxy)-8-azabicyclo[3.2.1]octan-8-yl)nicotinic acid (68b). To a mixture of methyl 6-((1R,3R,5S)-3-((5-cyclopropyl-3-(2-(trifluoromethoxy)phenyl)isoxazol-4-yl) methoxy)-8-azabicyclo[3.2.1]octan-8-yl)nicotinate (68a) (60 mg, 110.39 umol) in H₂O (1 mL), THF (1 mL) and methanol (1 mL) was added LiOH H₂O (27.79 mg, 662.33 umol) at 20° C. under N₂. The mixture was stirred at 35° C. for 16 hours and was acidified with 1N HCl to pH=5. The residue was concentrated under reduced pressure to give crude 68b. MS mass calculated for [M+H]⁺(C₂₇H₂₆F₃N₃O₅) requires m/z, 530.2, LCMS found m/z, 530.2.

Tert-butyl 2-(6-((1R,3R,5S)-3-((5-cyclopropyl-3-(2-(trifluoromethoxy)phenyl)isoxazol-4-yl)methoxy)-8-azabicyclo[3.2.1]octan-8-yl)nicotinoyl)hydrazinecarboxylate (68c). To a mixture of 6-((1R,3R,5S)-3-((5-cyclopropyl-3-(2-(trifluoromethoxy)phenyl)isoxazol-4-yl)methoxy)-8-azabicyclo[3.2.1]octan-8-yl)nicotinic acid (68b) (58 mg, 109.54 umol) and tert-butyl hydrazine carboxylate (28.95 mg, 219.07 umol) in DMF (2 mL) was added EDCI (27.30 mg, 142.40 umol) and DMAP (267.64 ug, 2.19 umol) at 20° C. under N₂. The mixture was stirred at 20° C. for 4 hours and was poured into ice-water (20 mL). The aqueous phase was extracted with ethyl acetate (10 mL*2). The combined organic phase was washed with brine (10 mL*2), dried with anhydrous Na₂SO₄, filtered and the filtrate was concentrated under reduced pressure. The residue was purified by prep-TLC to give 68c. MS mass calculated for [M+H]⁺(C₃₂H₃₆F₃N₅O₆) requires m/z, 644.2/645.2, LCMS found m/z, 644.2/645.2; ¹H NMR (400 MHz, CHLOROFORM-d) δ=8.50 (d, J=2.2 Hz, 1H), 7.76 (dd, J=2.4, 8.9 Hz, 1H), 7.58-7.40 (m, 3H), 7.34-7.28 (m, 2H), 6.36 (d, J=9.0 Hz, 1H), 4.43-4.28 (m, 2H), 4.27-4.19 (m, 2H), 3.44-3.32 (m, 1H), 3.01 (s, 1H), 2.04 (tt, J=5.1, 8.4 Hz, 1H), 1.92-1.72 (m, 6H), 1.60 (br d, J=14.9 Hz, 2H), 1.45-1.40 (m, 9H), 1.19-1.11 (m, 2H), 1.07-0.98 (m, 2H).

6-((1R,3R,5S)-3-((5-cyclopropyl-3-(2-(trifluoromethoxy)phenyl)isoxazol-4-yl)methoxy)-8-azabicyclo[3.2.1]octan-8-yl)nicotinohydrazide (68d). To a mixture of tert-butyl 2-(6-((1R,3R,5S)-3-((5-cyclopropyl-3-(2-(trifluoromethoxy)phenyl)isoxazol-4-yl)methoxy)-8-azabicyclo[3.2.1]octan-8-yl)nicotinoyl)hydrazinecarboxylate (68c) (40 mg, 62.15 umol) in ethyl acetate (0.5 mL) was added HCl/ethyl acetate (4 mL, 4 M) at 20° C. under N₂. The mixture was stirred at 20° C. for 1 hour and was concentrated under reduced pressure to give 68d. MS mass calculated for [M+H]⁺(C₂₇H₂₈F₃N₅O₄) requires m/z, 544.2, LCMS found m/z, 544.2.

5-(6-((1R,3R,5S)-3-((5-cyclopropyl-3-(2-(trifluoromethoxy)phenyl)isoxazol-4-yl)methoxy)-8-azabicyclo[3.2.1]octan-8-yl)pyridin-3-yl)-1,3,4-oxadiazol-2(3H)-one (Compound 68). To a mixture of 6-((1R,3R,5S)-3-((5-cyclopropyl-3-(2-(trifluoromethoxy)phenyl)isoxazol-4-yl)methoxy)-8-azabicyclo[3.2.1]octan-8-yl)nicotinohydrazide (68d) (33 mg, 60.71 umol) in THE (3 mL) was added CDI (19.69 mg, 121.43 umol) and TEA (18.43 mg, 182.14 umol, 25.35 uL) at 20° C. under N₂. The mixture was stirred at 20° C. for 6 hours and was poured into water (10 mL). The mixture was extracted with ethyl acetate (20 mL*2). The combined organic phase was washed with brine (10 mL), dried with anhydrous Na₂SO₄, filtered and the filtrate was concentrated under reduced pressure. The residue was purified by prep-HPLC (TFA condition) and lyophilized to give Compound 68. MS mass calculated for [M+H]⁺(MS mass calculated for [M+H]⁺(C₂₈H₂₆F₃N₅O₅) requires m/z, 570.2, LCMS found m/z, 570.2; ¹H NMR (400 MHz, CHLOROFORM-d) δ=9.95 (br s, 1H), 8.36 (s, 1H), 8.08-7.93 (m, 1H), 7.58-7.49 (m, 2H), 7.40 (t, J=7.2 Hz, 2H), 6.81 (d, J=9.5 Hz, 1H), 4.57 (br s, 2H), 4.36 (s, 2H), 3.57 (br s, 1H), 2.11-1.90 (m, 7H), 1.90-1.81 (m, 2H), 1.27-1.20 (m, 2H), 1.17-1.09 (m, 2H).

### Example 69

### 5-(6-((1R,3R,5S)-3-((5-cyclopropyl-3-(2-(trifluoromethoxy)phenyl)isoxazol-4-yl)methoxy)-8-azabicyclo[3.2.1]octan-8-yl)pyridin-3-yl)isoxazol-3(2H)-one

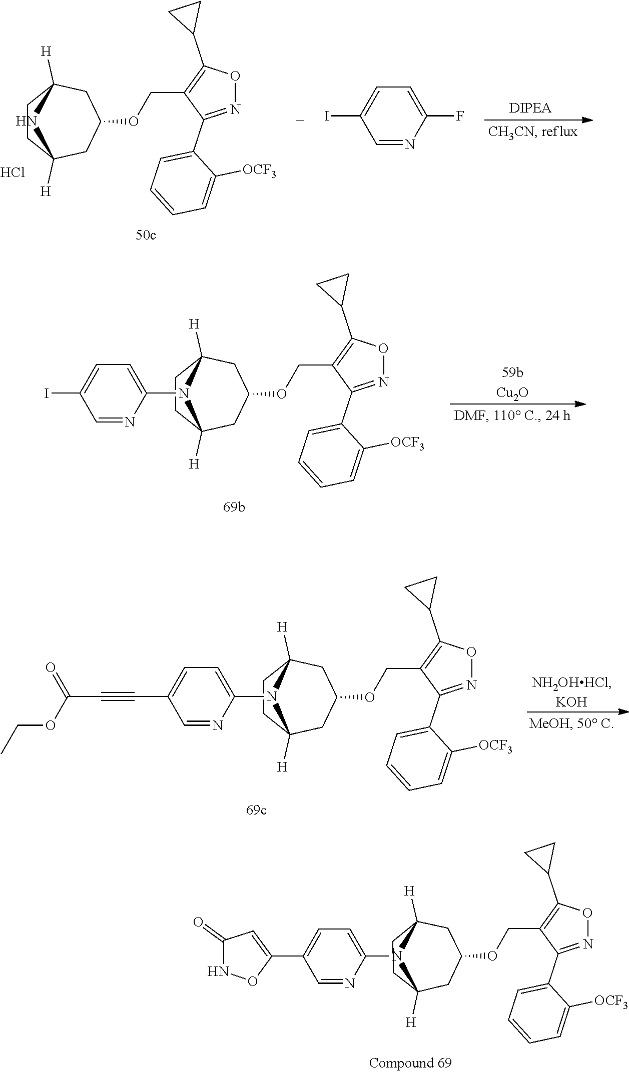

5-cyclopropyl-4-((((1R,3R,5S)-8-(5-iodopyridin-2-yl)-8-azabicyclo[3.2.1]octan-3-yl)oxy)methyl)-3-(2-(trifluoromethoxy)phenyl)isoxazole (69b). To a solution of 4-(((1R,3R,5S)-8-azabicyclo[3.2.1]octan-3-yloxy)methyl)-5-cyclopropyl-3-(2-(trifluoromethoxy)phenyl)isoxazole hydrochloride (50c) (200 mg, 449.57 umol) in acetronitrile (5 mL) was added DIPEA (290.51 mg, 2.25 mmol, 391.53 uL) and 2-fluoro-5-iodopyridine (69a) (300.74 mg, 1.35 mmol) in a sealed tube. The mixture was heated to 100° C. and stirred for 48 hours and was concentrated under reduced pressure. The residue was diluted with water (10 mL) and extracted with ethyl acetate (15 mL*2). The combined organic phase was washed with brine (10 mL), dried over anhydrous Na₂SO₄, filtered and the filtrate was concentrated. The residue was purified by prep-TLC (SiO₂, petroleum ether:ethyl acetate=3:1) to give 69b. MS mass calculated for [M+H]⁺(C₂₆H₂₅F₃₁N₃O₃) requires m/z, 612.1/613.1, LCMS found m/z, 612.0/613.0; ¹H NMR (400 MHz, CHLOROFORM-d) δ=8.27 (d, J=2.5 Hz, 1H), 7.64-7.46 (m, 3H), 7.39 (br t, J=7.3 Hz, 2H), 6.34 (d, J=8.5 Hz, 1H), 4.31 (s, 2H), 4.27 (br s, 2H), 3.51-3.38 (m, 1H), 2.18-2.06 (m, 1H), 2.00-1.74 (m, 6H), 1.61 (br d, J=14.6 Hz, 2H), 1.28-1.19 (m, 2H), 1.16-1.06 (m, 2H).

Ethyl 3-(6-((1R,3R,5S)-3-((5-cyclopropyl-3-(2-(trifluoromethoxy)phenyl)isoxazol-4-yl)methoxy)-8-azabicyclo[3.2.1]octan-8-yl)pyridin-3-yl)propiolate (69c). To a solution of 5-cyclopropyl-4-((((1R,3R,5S)-8-(5-iodopyridin-2-yl)-8-azabicyclo[3.2.1]octan-3-yl)oxy)methyl)-3-(2-(trifluoromethoxy)phenyl)isoxazole (69b) (80 mg, 130.85 umol) and ethyl prop-2-ynoate (128.36 mg, 1.31 mmol, 128.36 uL) in DMF (2 mL) was added Cu₂O (3.74 mg, 26.17 umol, 2.67 uL) in sealed tube. The mixture was heated to 110° C. and stirred for 18 hours. The reaction mixture was filtered through a Celite pad and rinsed with ethyl acetate (30 mL). The combined filtrate was washed with water (10 mL) and brine (10 mL*2) and was dried over anhydrous Na₂SO₄ and filtered. The filtrate was concentrated under reduced pressure. The residue was purified by Prep-TLC (SiO₂, petroleum ether:ethyl acetate=3:1) and by Prep-TLC (SiO₂, petroleum ether:ethyl acetate=1:1) to give 69c. MS mass calculated for [M+H]⁺(C₃₁H₃₀F₃N₃O₅) requires m/z, 582.2/583.2, LCMS found m/z, 582.2.2/583.2.

5-(6-((1R,3R,5S)-3-((5-cyclopropyl-3-(2-(trifluoromethoxy)phenyl)isoxazol-4-yl)methoxy)-8-azabicyclo[3.2.1]octan-8-yl)pyridin-3-yl)isoxazol-3(2H)-one (Compound 69). To a solution of ethyl 3-(6-((1R,3R,5S)-3-((5-cyclopropyl-3-(2-(trifluoromethoxy)phenyl)isoxazol-4-yl)methoxy)-8-azabicyclo[3.2.1]octan-8-yl)pyridin-3-yl)propiolate (69c) (20 mg, 34.39 umol) in methanol (2 mL) was added hydroxylamine hydrochloride (23.90 mg, 343.89 umol) and KOH (34.73 mg, 619.00 umol). The resulting mixture was heated to 50° C. and stirred for 18 hours and was concentrated under reduced pressure. The residue was purified by prep-TLC (SiO₂, dichloromethane:Methanol=10:1), then repurified by prep-HPLC (TFA, column: Phenomenex Synergi C18 150*25*10 um; mobile phase: [water (0.10% TFA)-ACN]; B %: 35%-65%, 8 min) to give Compound 69. MS mass calculated for [M+H]⁺(C₂₉H₂₇F₃N₄O₅) requires m/z, 569.2/570.2, LCMS found m/z, 569.2/570.2; ¹H NMR (400 MHz, CHLOROFORM-d) δ=8.30 (br s, 1H), 7.83 (br d, J=9.3 Hz, 1H), 7.60-7.48 (m, 2H), 7.40 (br t, J=7.2 Hz, 2H), 6.77 (br d, J=9.5 Hz, 1H), 6.08 (s, 1H), 4.53 (br s, 2H), 4.36 (s, 2H), 3.57 (br s, 1H), 2.18-1.88 (m, 7H), 1.83 (br d, J=14.8 Hz, 2H), 1.29-1.19 (m, 2H), 1.18-1.05 (m, 2H).

### Example 70

### 3-(3-(4-((5-cyclopropyl-3-(2,6-dichlorophenyl)isoxazol-4-yl)methoxy)piperidin-1-yl)-1-methyl-1H-pyrazol-5-yl)-1,2,4-oxadiazol-5(4H)-one

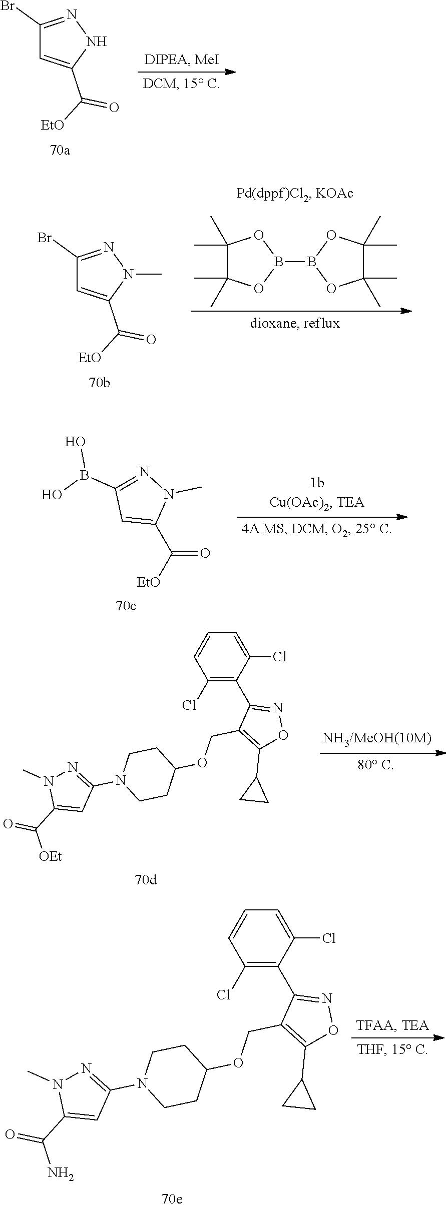

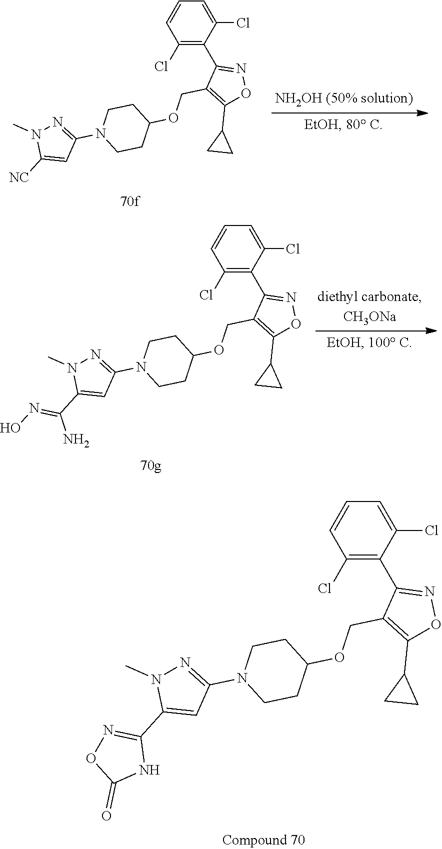 [SMILES: Cn1nc(N2CCC(OCc3c(-c4c(Cl)cccc4Cl)noc3C3CC3)CC2)cc1C#N ; Cn1nc(N2CCC(OCc3c(-c4c(Cl)cccc4Cl)noc3C3CC3)CC2)cc1/C(N)=N/O ; Cn1nc(N2CCC(OCc3c(-c4c(Cl)cccc4Cl)noc3C3CC3)CC2)cc1-c1noc(=O)[nH]1]

Ethyl 3-bromo-1-methyl-1H-pyrazole-5-carboxylate (70b). To a solution of ethyl 3-bromo-1H-pyrazole-5-carboxylate (70a) (1 g, 4.57 mmol) in dichloromethane (10 mL) was added DIPEA (1.18 g, 9.13 mmol, 1.59 mL) and Mel (1.30 g, 9.13 mmol, 568.43 uL) dropwise at 15° C. The reaction mixture was stirred for 18 hours. The reaction mixture was diluted with water (5 mL) and extracted with dichloromethane (10 mL*2). The combined organic phase was washed with brine (5 mL*2), dried over anhydrous sodium sulfate, filtered and the filtrate was concentrated. The residue was purified by prep-TLC (SiO₂, petroleum ether:ethyl acetate=3:1) to give 70b. MS mass calculated for [M+H]⁺(C₇H₉BrN₂O₂) requires m/z, 233.0/235.0, LCMS found m/z, 232.9/234.9; ¹H NMR (400 MHz, CHLOROFORM-d) δ=6.83 (s, 1H), 4.36 (q, J=6.8 Hz, 2H), 4.16 (s, 3H), 1.38 (t, J=7.1 Hz, 3H).

(5-(ethoxycarbonyl)-1-methyl-1H-pyrazol-3-yl)boronic acid (70c). To a solution of ethyl 3-bromo-1-methyl-1H-pyrazole-5-carboxylate (70b) (200 mg, 858.14 umol) and 4,4,5,5-tetramethyl-2-(4,4,5,5-tetramethyl-1,3,2-dioxaborolan-2-yl)-1,3,2-dioxaborolane (1.09 g, 4.29 mmol) in 1,4-dioxane (5 mL) was added Pd(dppf)Cl₂ (125.58 mg, 171.63 umol) and KOAc (168.44 mg, 1.72 mmol). The resulting mixture was degassed and purged with N₂ three times and heated to reflux and stirred for 18 hours. The reaction mixture was cooled to 45° C. and diluted with ethyl acetate (10 ml). 3-Mercaptopropyl-functionalized silica gel (2 g) was added and the mixture was stirred for 2 hour at 45° C. The mixture was filtered through a Celite pad and the filtrate was concentrated under reduced pressure. The residue was purified by prep-HPLC (TFA condition; column: Phenomenex luna C18 250*50 mm*10 um; mobile phase: [water (0.1% TFA)-ACN]; B %: 1%-40%, 10 min) to give 70c. MS mass calculated for [M+H]⁺(C₇H₁₁BN₂O₄) requires m/z, 199.1/198.1, LCMS found m/z, 199.1/198.1; ¹H NMR (400 MHz, CHLOROFORM-d) δ=7.50 (s, 1H), 7.30 (s, 1H), 6.97 (s, 1H), 4.42-4.34 (m, 2H), 4.29 (s, 1H), 4.26 (s, 2H), 1.46-1.36 (m, 3H).

Ethyl3-(4-((5-cyclopropyl-3-(2,6-dichlorophenyl)isoxazol-4-yl)methoxy)piperidin-1-yl)-1-methyl-1H-pyrazole-5-carboxylate (70d). To a solution of (5-(ethoxycarbonyl)-1-methyl-1H-pyrazol-3-yl)boronic acid (70c) (140 mg, 707.13 umol) and 5-cyclopropyl-3-(2,6-dichlorophenyl)-4-((piperidin-4-yloxy)methyl)isoxazole hydrochloride (1b) (259.71 mg, 707.13 umol) in dichloromethane (5 mL) was added Cu(OAc)₂ (128.44 mg, 707.13 umol), TEA (214.66 mg, 2.12 mmol, 295.27 uL) and molecular sieves 4 A (50 mg). The resulting mixture was degassed and purged with O₂ three times and stirred for 18 hours at 25° C. The reaction mixture was filtered through a Celite pad and the filter cake was rinsed with dichloromethane (10 mL*2). The combined filtrate was concentrated under reduced pressure. The residue was purified by prep-TLC (SiO₂, petroleum ether:ethyl acetate=1:1) to give Compound 70d. MS mass calculated for [M+H]⁺(C₂₅H₂₈Cl₂N₄O₄) requires m/z, 519.2/521.2, LCMS found m/z, 519.1/521.1; ¹H NMR (400 MHz, CHLOROFORM-d) δ=7.45-7.37 (m, 2H), 7.35-7.28 (m, 1H), 6.16 (s, 1H), 4.38-4.27 (m, 4H), 4.02 (s, 3H), 3.42-3.30 (m, 3H), 2.79 (ddd, J=3.4, 9.5, 12.5 Hz, 2H), 2.17 (ddd, J=3.4, 5.1, 8.6 Hz, 1H), 1.82-1.71 (m, 2H), 1.55-1.46 (m, 2H), 1.37 (t, J=7.3 Hz, 3H), 1.30-1.24 (m, 2H), 1.17-1.08 (m, 2H)

3-(4-((5-cyclopropyl-3-(2,6-dichlorophenyl)isoxazol-4-yl)methoxy)piperidin-1-yl)-1-methyl-1H-pyrazole-5-carboxamide (70e). A solution of ethyl 3-(4-((5-cyclopropyl-3-(2,6-dichlorophenyl)isoxazol-4-yl)methoxy)piperidin-1-yl)-1-methyl-1H-pyrazole-5-carboxylate (70d) (110 mg, 211.78 umol) in NH₃/MeOH (15 mL, 10 M) was stirred at 80° C. for 18 hours in sealed tube. The reaction mixture was concentrated under reduced pressure. The residue was combined with another batch and purified by prep-TLC (SiO₂, dichloromethane:MeOH=10:1) to give 70e. MS mass calculated for [M+H]⁺(C₂₃H₂₅Cl₂N₅O₃) requires m/z, 490.1/492.1, LCMS found m/z, 490.1/492.1; ¹H NMR (400 MHz, CHLOROFORM-d) δ=7.45-7.36 (m, 2H), 7.34-7.29 (m, 1H), 5.86 (s, 1H), 4.34 (s, 2H), 4.02 (s, 3H), 3.43-3.29 (m, 3H), 2.86-2.74 (m, 2H), 2.21-2.12 (m, 1H), 1.76 (br s, 2H), 1.54-1.44 (m, 2H), 1.31-1.24 (m, 2H), 1.18-1.07 (m, 2H).

3-(4-((5-cyclopropyl-3-(2,6-dichlorophenyl)isoxazol-4-yl)methoxy)piperidin-1-yl)-1-methyl-1H-pyrazole-5-carbonitrile (70f). To a solution of 3-(4-((5-cyclopropyl-3-(2,6-dichlorophenyl)isoxazol-4-yl)methoxy)piperidin-1-yl)-1-methyl-1H-pyrazole-5-carboxamide (70e) (80 mg, 163.14 umol) in THF (5 mL) was added TEA (99.05 mg, 978.83 umol, 136.24 uL,) and TFAA (102.79 mg, 489.42 umol, 68.07 uL) dropwise at 0° C. The mixture was stirred for 10 min at 15° C. and was concentrated under reduced pressure. The residue was diluted with dichloromethane (20 mL) and washed with sodium bicarbonate solution (10 mL). The organic phase was washed with brine (10 mL), dried over anhydrous sodium sulfate, filtered and concentrated to give 70f. MS mass calculated for [M+H]⁺(C₂₃H₂₃Cl₂N₅O₂) requires m/z, 472.1/474.1, LCMS found m/z, 472.1/474.1.

(Z)-3-(4-((5-cyclopropyl-3-(2,6-dichlorophenyl)isoxazol-4-yl)methoxy)piperidin-1-yl)-N′-hydroxy-1-methyl-1H-pyrazole-5-carboximidamide (70g). To a solution of 3-(4-((5-cyclopropyl-3-(2,6-dichlorophenyl)isoxazol-4-yl)methoxy)piperidin-1-yl)-1-methyl-1H-pyrazole-5-carbonitrile (70f) (80 mg, 169.36 umol) in EtOH (2 mL) was added hydroxylamine (1 mL, 50% purity). The mixture was heated to 80° C. and stirred for 2 hours and was concentrated under reduced pressure. The residue was purified by Prep-TLC (dichloromethane:methanol=10:1) to give 70g. MS mass calculated for [M+H]⁺(C₂₃H₂₆Cl₂N₆O₃) requires m/z, 505.1/507.1, LCMS found m/z, 505.1/507.1; ¹H NMR (400 MHz, CHLOROFORM-d) δ=7.46-7.37 (m, 2H), 7.34 (br d, J=7.1 Hz, 1H), 6.00 (br s, 1H), 4.34 (s, 2H), 3.99 (br s, 3H), 3.35 (m, 3H), 2.83 (br s, 2H), 2.16 (br s, 1H), 1.76 (br s, 2H), 1.52 (br d, J=8.8 Hz, 2H), 1.31-1.20 (m, 2H), 1.18-1.08 (m, 2H).

3-(3-(4-((5-cyclopropyl-3-(2,6-dichlorophenyl)isoxazol-4-yl)methoxy)piperidin-1-yl)-1-methyl-1H-pyrazol-5-yl)-1,2,4-oxadiazol-5(4H)-one (Compound 70). To a solution of (Z)-3-(4-((5-cyclopropyl-3-(2,6-dichlorophenyl)isoxazol-4-yl)methoxy)piperidin-1-yl)-N′-hydroxy-1-methyl-1H-pyrazole-5-carboximidamide (70g) (70 mg, 138.51 umol) in EtOH (2 mL) was added CH₃ONa (149.65 mg, 831.03 umol, 30% in MeOH) and diethyl carbonate (981.71 mg, 8.31 mmol, 1.01 mL) in sealed tube. The mixture was heated to 100° C. and stirred for 1 hour and concentrated under reduced pressure. The residue was diluted with water (10 mL) and extracted with dichloromethane (20 mL*2). The combined organic phase was washed with brine (10 mL*3), dried over anhydrous Sodium sulfate, filtered and concentrated. The residue was purified by prep-HPLC (neutral condition: column: Waters Xbridge Prep OBD C_{18 150*40} mm*10 um; mobile phase: [water (10 mM NH₄HCO₃)-ACN]; B %: 20%-50%, 8 min) to give Compound 70. MS mass calculated for [M+H]⁺(C₂₄H₂₄Cl₂N₆O₄) requires m/z, 531.1/533.1, LCMS found m/z, 517.1/519.1; ¹H NMR (400 MHz, CHLOROFORM-d) δ=7.46-7.37 (m, 2H), 7.34 (br d, J=7.1 Hz, 1H), 6.00 (br s, 1H), 4.34 (s, 2H), 3.99 (br s, 3H), 3.35 (br s, 3H), 2.83 (br s, 2H), 2.16 (br s, 1H), 1.76 (br s, 2H), 1.52 (br d, J=8.8 Hz, 2H), 1.31-1.20 (m, 2H), 1.18-1.08 (m, 2H).

### Example 71

### 3-(6-((2R,4R)-4-((5-cyclopropyl-3-(2,6-dichlorophenyl)isoxazol-4-yl)methoxy)-2-methylpiperidin-1-yl)pyridin-3-yl)-1,2,4-oxadiazol-5(4H)-one

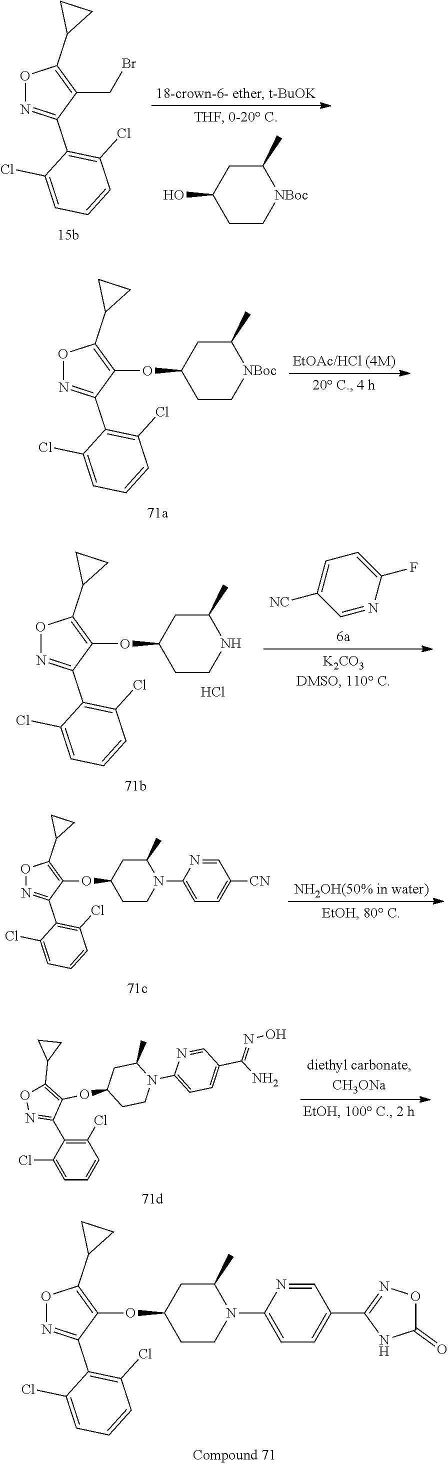

(2R,4R)-tert-butyl 4-((5-cyclopropyl-3-(2,6-dichlorophenyl)isoxazol-4-yl)methoxy)-2-methylpiperidine-1-carboxylate (71a). To a solution of (2R,4R)-tert-butyl 4-hydroxy-2-methylpiperidine-1-carboxylate (62 mg, 287.99 umol) in THF (8 mL) was added 18-CROWN-6 (114.18 mg, 431.98 umol), t-BuOK (1 M in THF, 431.98 uL) at 0° C. The reaction was degassed and purged with N₂ 3 times and stirred at 25° C. for 0.5 hour under N₂ atmosphere. Then 4-(bromomethyl)-5-cyclopropyl-3-(2,6-dichlorophenyl)isoxazole (15b) (99.94 mg, 287.99 umol) was added and the mixture was stirred at 20° C. for 3.5 hours. The reaction mixture was concentrated under reduced pressure and water (5 mL) and ethyl acetate (5 mL) were added to the residue. The phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, petroleum ether:ethyl acetate=3:1, R_{f}=0.31) to give 71a. ¹H NMR (400 MHz, CHLOROFORM-d) δ=7.43-7.37 (m, 2H), 7.36-7.31 (m, 1H), 4.32-4.22 (m, 2H), 3.69 (br d, J=16.2 Hz, 1H), 3.57 (t, J=3.3 Hz, 1H), 3.00-2.91 (m, 1H), 2.18-2.10 (m, 1H), 1.63-1.57 (m, 3H), 1.53-1.47 (m, 1H), 1.44 (s, 9H), 1.29-1.24 (m, 3H), 1.15-1.10 (m, 2H), 1.08 (d, J=7.0 Hz, 3H).

5-cyclopropyl-3-(2,6-dichlorophenyl)-4-((((2R,4R)-2-methylpiperidin-4-yl)oxy)methyl)isoxazole (71b). To a solution of (2R,4R)-tert-butyl 4-((5-cyclopropyl-3-(2,6-dichlorophenyl)isoxazol-4-yl)methoxy)-2-methylpiperidine-1-carboxylate (71a) (120 mg, 249.27 umol) in ethyl acetate (2 mL) was added ethyl acetate/HCl (5 mL) at 20° C. and the mixture was stirred for 4 hours. White solid was precipitated and the reaction mixture was concentrated to give 71b.

6-((2R,4R)-4-((5-cyclopropyl-3-(2,6-dichlorophenyl)isoxazol-4-yl)methoxy)-2-methylpiperidin-1-yl)nicotinonitrile (71c). To a solution of 5-cyclopropyl-3-(2,6-dichlorophenyl)-4-((((2R,4R)-2-methylpiperidin-4-yl)oxy)methyl)isoxazole hydrochloride (71b) (45 mg, 107.72 umol) and 6-fluoronicotinonitrile (6a) (65.76 mg, 538.60 umol) in DMSO (3 mL) was added K₂CO₃ (89.32 mg, 646.32 umol) at 20° C. The suspension was degassed under vacuum and purged with N₂ several times. The mixture was stirred at 110° C. for 16 hours and was concentrated under reduced pressure. Water (5 mL) and ethyl acetate (5 mL) were added into the residue and the phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, petroleum ether:ethyl acetate=2:1, R_{f}=0.50) to give 71c. MS mass calculated for [M+H]⁺(C₂₅H₂₄Cl₂N₄O₂) requires m/z, 483.1/485.1, LCMS found m/z 483.2/485.2; ¹H NMR (400 MHz, CHLOROFORM-d) δ=8.37 (d, J=2.2 Hz, 1H), 7.55 (dd, J=2.3, 9.1 Hz, 1H), 7.44-7.39 (m, 2H), 7.37-7.32 (m, 1H), 6.49 (d, J=9.2 Hz, 1H), 4.51-4.42 (m, 1H), 4.38-4.28 (m, 2H), 4.08 (br d, J=12.7 Hz, 1H), 3.65 (t, J=3.4 Hz, 1H), 3.14 (dt, J=2.8, 13.1 Hz, 1H), 2.35-2.27 (m, 1H), 2.18-2.11 (m, 1H), 1.77-1.72 (m, 2H), 1.64-1.57 (m, 1H), 1.30-1.26 (m, 2H), 1.17-1.10 (m, 5H).

(Z)-6-((2R,4R)-4-((5-cyclopropyl-3-(2,6-dichlorophenyl)isoxazol-4-yl)methoxy)-2-methylpiperidin-1-yl)-N′-hydroxynicotinimidamide (71d). To a solution of 6-((2R,4R)-4-((5-cyclopropyl-3-(2,6-dichlorophenyl)isoxazol-4-yl)methoxy)-2-methylpiperidin-1-yl)nicotinonitrile (71c) (40 mg, 82.75 umol) in ethanol (2 mL) was added hydroxylamine (16.40 mg, 248.25 umol, 2 mL, 50% in H₂O) at 20° C. The reaction was degassed and purged with N₂ 3 times and was stirred at 80° C. for 2 hours under N₂ atmosphere. The reaction mixture was concentrated under reduced pressure to remove solvent. Water (5 mL) and ethyl acetate (5 mL) were added into the residue and the phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, petroleum ether:ethyl acetate=0:1, R_{f}=0.13) to give 71d. MS mass calculated for [M+H]⁺(C₂₅H₂₇Cl₂N₅O₃) requires m/z, 516.2/518.2, LCMS found m/z 516.2/518.2; ¹H NMR (400 MHz, CHLOROFORM-d) δ=8.37 (d, J=2.4 Hz, 1H), 7.68 (dd, J=2.5, 9.0 Hz, 1H), 7.44-7.40 (m, 2H), 7.36-7.31 (m, 1H), 6.53 (d, J=9.2 Hz, 1H), 4.75 (br s, 2H), 4.45 (br s, 1H), 4.38-4.28 (m, 2H), 3.99 (br d, J=12.0 Hz, 1H), 3.64 (t, J=3.3 Hz, 1H), 3.11 (br t, J=12.9 Hz, 1H), 2.20-2.11 (m, 1H), 1.75 (br d, J=3.7 Hz, 2H), 1.72 (br s, 1H), 1.65 (br d, J=4.3 Hz, 1H), 1.29-1.26 (m, 2H), 1.16-1.11 (m, 5H).

3-(6-((2R,4R)-4-((5-cyclopropyl-3-(2,6-dichlorophenyl)isoxazol-4-yl)methoxy)-2-methylpiperidin-1-yl)pyridin-3-yl)-1,2,4-oxadiazol-5(4H)-one (Compound 71). A mixture of (Z)-6-((2R,4R)-4-((5-cyclopropyl-3-(2,6-dichlorophenyl)isoxazol-4-yl)methoxy)-2-methylpiperidin-1-yl)-N′-hydroxynicotinimidamide (71d) (35 mg, 67.77 umol) in ethanol (2 mL) was added diethyl carbonate (975.00 mg, 8.25 mmol, 1 mL) and CH₃ONa (73.22 mg, 406.65 umol, 0.2 mL, 30% in MeOH) at 20° C. The reaction was degassed and purged with N₂ 3 times and was stirred at 100° C. for 4 hours. The reaction mixture was concentrated under reduced pressure and water (5 mL) and ethyl acetate (5 mL) were added into the residue. The phases were separated. The aqueous phase was extracted with ethyl acetate (5 mL*4). The combined organic phase was washed with brine (20 mL*2), dried with anhydrous Na₂SO₄, filtered and concentrated under reduced pressure and purified by prep-HPLC (neutral condition: column: Waters Xbridge BEH C18 100*25 mm*5 um; mobile phase: [water(10 mM NH₄HCO₃)-ACN]; B %: 20%-55%, 10 min) to give Compound 71. MS mass calculated for [M+H]⁺(C₂₆H₂₅Cl₂N₅O₄) requires m/z, 542.1/544.1, LCMS found m/z 542.1/544.1; ¹H NMR (400 MHz, CHLOROFORM-d) δ=8.48 (d, J=2.4 Hz, 1H), 7.75 (dd, J=2.5, 9.2 Hz, 1H), 7.44-7.39 (m, 2H), 7.38-7.32 (m, 1H), 6.59 (d, J=9.3 Hz, 1H), 4.55-4.45 (m, 1H), 4.39-4.28 (m, 2H), 4.11 (br d, J=11.9 Hz, 1H), 3.66 (t, J=3.3 Hz, 1H), 3.15 (dt, J=2.8, 13.1 Hz, 1H), 2.16 (tt, J=5.1, 8.4 Hz, 1H), 1.82-1.70 (m, 3H), 1.67-1.56 (m, 1H), 1.31-1.25 (m, 2H), 1.17 (d, J=6.8 Hz, 3H), 1.16-1.11 (m, 2H).

### Example 72

### 3-(6-((2R,4R)-4-((3-(2-chlorophenyl)-5-cyclopropylisoxazol-4-yl)methoxy)-2-methylpiperidin-1-yl)pyridin-3-yl)-1,2,4-oxadiazol-5(4H)-one

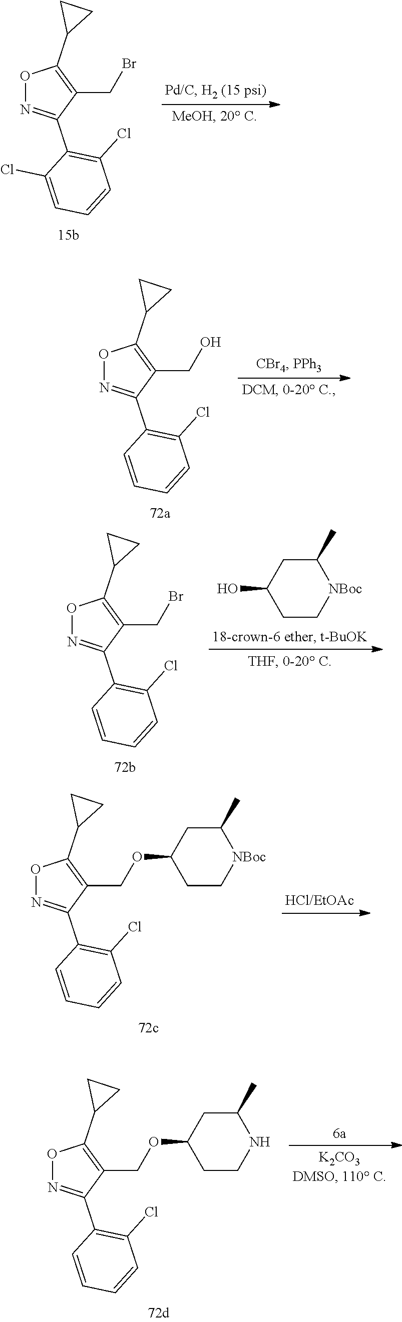 [SMILES: Clc1cccc(Cl)c1-c1noc(C2CC2)c1CBr ; OCc1c(-c2ccccc2Cl)noc1C1CC1 ; Clc1ccccc1-c1noc(C2CC2)c1CBr ; C[C@@H]1C[C@H](O)CCN1C(=O)OC(C)(C)C ; C[C@@H]1C[C@H](OCc2c(-c3ccccc3Cl)noc2C2CC2)CCN1C(=O)OC(C)(C)C ; C[C@@H]1C[C@H](OCc2c(-c3ccccc3Cl)noc2C2CC2)CCN1]

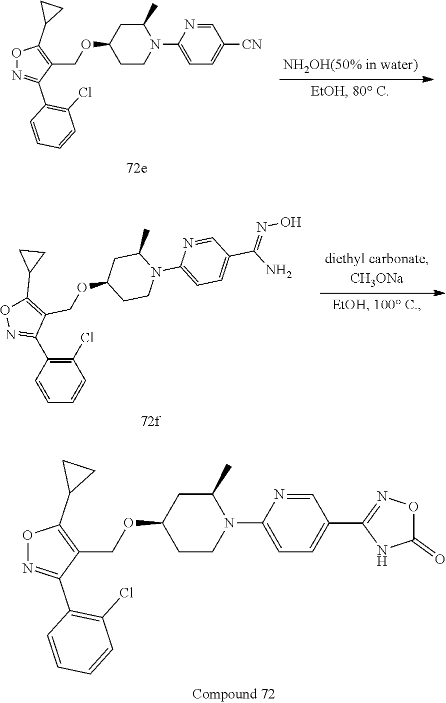 [SMILES: C[C@@H]1C[C@H](OCc2c(-c3ccccc3Cl)noc2C2CC2)CCN1c1ccc(C#N)cn1 ; C[C@@H]1C[C@H](OCc2c(-c3ccccc3Cl)noc2C2CC2)CCN1c1ccc(/C(N)=N/O)cn1 ; C[C@@H]1C[C@H](OCc2c(-c3ccccc3Cl)noc2C2CC2)CCN1c1ccc(-c2noc(=O)[nH]2)cn1]

(3-(2-chlorophenyl)-5-cyclopropylisoxazol-4-yl)methanol (72a). To a mixture of Pd/C (5 mg, 1.56 mmol, 10% purity) in MeOH (5 mL) was added (5-cyclopropyl-3-(2,6-dichlorophenyl)isoxazol-4-yl)methanol (15b) (500 mg, 1.56 mmol). The mixture was degassed and purged with H₂ 3 times and stirred at 20° C. for 1.5 hours under H₂ (15 psi). After 1.5 hours, the reaction mixture was filtered through a Celite pad. The filter cake was rinsed with MeOH (10 mL). The combined filtrate was concentrated under reduced pressure. The residue was purified by prep-HPLC (HCl condition: column: Phenomenex luna C18 80*40 mm*3 um; mobile phase: [water (0.04% HCl)-ACN]; B %: 27%-47%, 7 min) to give 72a. MS mass calculated for [M+H]⁺(C₁₃H₁₂ClNO₂) requires m/z, 250.1/252.1, LCMS found m/z, 250.0/252.0.

4-(bromomethyl)-3-(2-chlorophenyl)-5-cyclopropylisoxazole (72b). To a solution of (3-(2-chlorophenyl)-5-cyclopropylisoxazol-4-yl)methanol (72a) (60 mg, 240.30 umol) in dichloromethane (3 mL) was added PPh₃ (126.05 mg, 480.59 umol), followed by CBr₄ (119.53 mg, 360.44 umol) in portions. The reaction mixture was stirred at 20° C. for 6 hours and was poured into water (15 mL) and extracted with dichloromethane (20 mL*3). The combined organic layers were concentrated to give a residue. The residue was purified by prep-TLC to give 72b. MS mass calculated for [M+H]⁺(C₁₃HnBrClNO) requires m/z, 312.0/314.0, LCMS found m/z, 311.9/313.9; ¹HNMR (CHLOROFORM-d, 400 MHz): δ=7.54-7.37 (m, 4H), 4.33 (s, 2H), 2.13 (tt, J=5.1, 8.4 Hz, 1H), 1.32-1.26 (m, 2H), 1.25-1.17 (m, 2H).

(2R,4R)-tert-butyl 4-((3-(2-chlorophenyl)-5-cyclopropylisoxazol-4-yl)methoxy)-2-methylpiperidine-1-carboxylate (72c). To a solution of 18-CROWN-6 (88.79 mg, 335.90 umol) and tert-butyl (2R,4R)-4-hydroxy-2-methyl-piperidine-1-carboxylate (53.03 mg, 246.33 umol) in THE (8 mL) was added t-BuOK (1M in THF, 335.90 uL) at 0° C. The mixture was stirred at 20° C. for 0.5 hour and 4-(bromomethyl)-3-(2-chlorophenyl)-5-cyclopropylisoxazole (72b) (70 mg, 223.94 umol) was added. The mixture was stirred at 20° C. for 3.5 hours and was poured into water (5 mL) and extracted with ethyl acetate (5 mL*4). The combined organic layer was washed with brine (20 mL), dried over sodium sulfate, filtered and the filtrate was concentrated to give a residue, which was purified by prep-TLC (petroleum ether:ethyl acetate=3:1) to give 72c. MS mass calculated for [M+H]⁺(C₂₄H₃₁ClN₂O₄) requires m/z, 447.2/449.2, LCMS found m/z, 447.3/449.2; ¹H NMR (CHLOROFORM-d, 400 MHz): δ=7.51-7.46 (m, 1H), 7.45-7.38 (m, 2H), 7.37-7.32 (m, 1H), 4.32 (s, 2H), 4.17 (br s, 1H), 3.74-3.67 (m, 1H), 3.58 (t, J=3.2 Hz, 1H), 2.95 (dt, J=2.9, 13.2 Hz, 1H), 2.13 (tt, J=5.1, 8.4 Hz, 1H), 1.61 (br dd, J=3.2, 5.4 Hz, 2H), 1.47 (s, 2H), 1.44 (s, 9H), 1.27-1.24 (m, 2H), 1.13-1.08 (m, 2H), 1.07 (d, J=7.0 Hz, 3H).

3-(2-chlorophenyl)-5-cyclopropyl-4-((((2R,4R)-2-methylpiperidin-4-yl)oxy)methyl)isoxazole (72d). To a solution of (2R,4R)-tert-butyl 4-((3-(2-chlorophenyl)-5-cyclopropylisoxazol-4-yl)methoxy)-2-methylpiperidine-1-carboxylate (72c) (70 mg, 156.61 umol) in ethyl acetate (2 mL) was added HCl/EtOAc (5 mL, 4M) at 20° C. and the mixture was stirred for 4 hours. The reaction mixture was concentrated to give 72d.

6-((2R,4R)-4-((3-(2-chlorophenyl)-5-cyclopropylisoxazol-4-yl)methoxy)-2-methylpiperidin-1-yl)nicotinonitrile (72e). To a solution of 3-(2-chlorophenyl)-5-cyclopropyl-4-((((2R,4R)-2-methylpiperidin-4-yl)oxy)methyl)isoxazole hydrochloride (72d) (60 mg, 156.53 umol) and 6-fluoronicotinonitrile (6a) (95.56 mg, 782.65 umol) in DMSO (3 mL) was added K₂CO₃ (129.80 mg, 939.18 umol) at 20° C. The mixture was stirred at 110° C. for 16 hours in sealed tube and was diluted with ethyl acetate (5 mL) and washed with brine (20 mL*2). The organic layer was dried over sodium sulfate and concentrated. The residue was purified by prep-TLC (petroleum ether:ethyl acetate=2:1, R_{f}=0.50) to give 72e. MS mass calculated for [M+H]⁺(C₂₅H₂₅ClN₄O₂) requires m/z, 449.2/451.2, LCMS found m/z, 449.2/451.2; ¹H NMR (CHLOROFORM-d, 400 MHz): δ=8.29 (d, J=2.1 Hz, 1H), 7.47 (dd, J=2.4, 9.1 Hz, 1H), 7.44-7.40 (m, 1H), 7.37-7.31 (m, 2H), 7.29-7.24 (m, 1H), 6.41 (d, J=9.0 Hz, 1H), 4.38 (br s, 1H), 4.30 (s, 2H), 4.00 (br d, J=11.5 Hz, 1H), 3.58 (t, J=3.4 Hz, 1H), 3.03 (dt, J=2.9, 13.2 Hz, 1H), 2.10-2.02 (m, 1H), 1.73-1.64 (m, 3H), 1.57-1.49 (m, 1H), 1.19-1.16 (m, 2H), 1.08-1.01 (m, 5H).

(Z)-6-((2R,4R)-4-((3-(2-chlorophenyl)-5-cyclopropylisoxazol-4-yl)methoxy)-2-methylpiperidin-1-yl)-N′-hydroxynicotinimidamide (72f). To a solution of 6-((2R,4R)-4-((3-(2-chlorophenyl)-5-cyclopropylisoxazol-4-yl)methoxy)-2-methylpiperidin-1-yl)nicotinonitrile (72e) (55 mg, 122.51 umol) in ethanol (2 mL) was added hydroxylamine (24.28 mg, 367.53 umol, 1 mL, 50% in water) at 20° C. The mixture was stirred at 80° C. for 2 hours and was concentrated. The residue was diluted with ethyl acetate (5 mL) and washed with brine (20 mL*2). The organic layer was dried over sodium sulfate, filtered and the filtrate was concentrated. The residue was purified by prep-TLC (dichloromethane:methanol=10:1, R_{f}=0.13) to give 72f. MS mass calculated for [M+H]⁺(C₂₅H₂₈ClN₅O₃) requires m/z, 482.2/484.2, LCMS found m/z, 482.3/484.2; ¹H NMR (CHLOROFORM-d, 400 MHz): δ=8.37 (d, J=2.0 Hz, 1H), 7.68 (dd, J=2.3, 9.0 Hz, 1H), 7.53-7.47 (m, 1H), 7.46-7.38 (m, 2H), 7.36-7.30 (m, 1H), 6.52 (d, J=9.0 Hz, 1H), 4.76 (br s, 2H), 4.43 (br s, 1H), 4.37 (s, 2H), 3.99 (br d, J=14.5 Hz, 1H), 3.65 (br s, 1H), 3.09 (br t, J=11.9 Hz, 1H), 2.20-2.09 (m, 1H), 1.81-1.72 (m, 3H), 1.64 (br d, J=12.8 Hz, 1H), 1.25 (br d, J=2.6 Hz, 2H), 1.11 (br d, J=7.0 Hz, 5H).

3-(6-((2R,4R)-4-((3-(2-chlorophenyl)-5-cyclopropylisoxazol-4-yl)methoxy)-2-methylpiperidin-1-yl)pyridin-3-yl)-1,2,4-oxadiazol-5(4H)-one (Compound 72). To a solution of (Z)-6-((2R,4R)-4-((3-(2-chlorophenyl)-5-cyclopropylisoxazol-4-yl)methoxy)-2-methylpiperidin-1-yl)-N′-hydroxynicotinimidamide (72f) (45 mg, 93.37 umol) and diethyl carbonate (1.46 g, 12.38 mmol, 1.5 mL) in ethanol (3 mL) was added NaOMe (1.46 g, 12.38 mmol, 1.5 mL, 30% in MeOH) at 20° C. The mixture was stirred at 100° C. for 4 hours and was concentrated under reduced pressure to remove solvent. Water (5 mL) and ethyl acetate (5 mL) were added to the reaction mixture and phases were separated. The aqueous phase was extracted with ethyl acetate (5 mL*4). The combined organic phase was washed with brine (20 mL*2), dried with anhydrous Na₂SO₄, filtered and concentrated under reduced pressure to a residue which was purified by prep-HPLC (neutral condition: column: Phenomenex Gemini-NX C18 75*30 mm*3 um; mobile phase: [water(10 mM NH₄HCO₃)-ACN]; B %: 30%-50%, 8 min) to give Compound 72. MS mass calculated for [M+H]⁺(C₂₆H₂₆ClN₅O₄) requires m/z, 508.2/510.2, LCMS found m/z, 508.1/510.1; ¹H NMR (CHLOROFORM-d, 400 MHz): δ=8.48 (br s, 1H), 7.76 (br d, J=8.2 Hz, 1H), 7.53-7.48 (m, 1H), 7.46-7.38 (m, 2H), 7.37-7.31 (m, 1H), 6.59 (br d, J=8.8 Hz, 1H), 4.49 (br d, J=6.2 Hz, 1H), 4.39 (s, 2H), 4.10 (br d, J=13.5 Hz, 1H), 3.66 (t, J=3.2 Hz, 1H), 3.19-3.08 (m, 1H), 2.19-2.11 (m, 1H), 1.82-1.73 (m, 3H), 1.68-1.56 (m, 1H), 1.29-1.24 (m, 2H), 1.17-1.09 (m, 5H).

### Example 73

### 3-(6-((2R,4R)-4-((5-cyclopropyl-3-(2,6-difluorophenyl)isoxazol-4-yl)methoxy)-2-methylpiperidin-1-yl)pyridin-3-yl)-1,2,4-oxadiazol-5(4H)-one

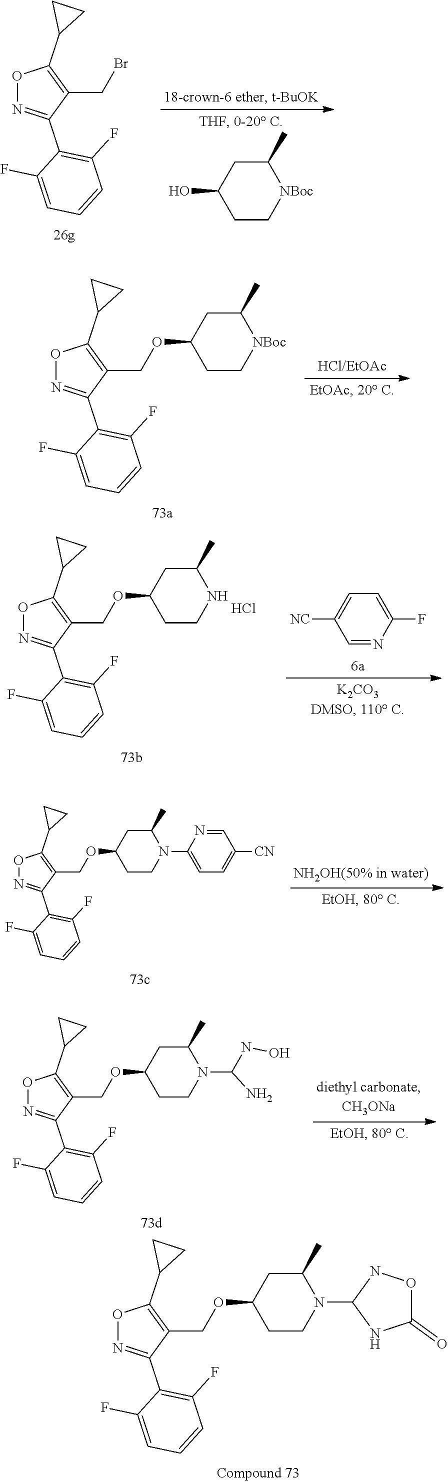

(2R,4R)-tert-butyl 4-((5-cyclopropyl-3-(2,6-difluorophenyl)isoxazol-4-yl)methoxy)-2-methylpiperidine-1-carboxylate (73a). To a solution of (2R,4R)-tert-butyl 4-hydroxy-2-methylpiperidine-1-carboxylate (205.61 mg, 955.03 umol) in THE (5 mL) was added 18-Crown-6 ether (378.65 mg, 1.43 mmol) and t-BuOK (1 M, 1.91 mL) at 0° C. The mixture was stirred at 20° C. for 30 min then 4-(bromomethyl)-5-cyclopropyl-3-(2,6-difluorophenyl)isoxazole (26g) (300 mg, 955.03 umol) in THE (5 mL) was added dropwise to the mixture at 20° C. The mixture was stirred at 20° C. for 2 hours and was poured into H₂O (15 mL) and extracted with ethyl acetate (15 mL*3). The combined organic layer was washed with brine (10 mL), dried over Na₂SO₄, filtered and the filtrate was concentrated under reduced pressure to give a residue. The residue was purified by column chromatography (SiO₂, petroleum ether:ethyl acetate=50:1 to 5:1) to give 73a. ¹H NMR (400 MHz, CHLOROFORM-d) δ=7.48-7.38 (m, 1H), 7.02 (t, J=7.8 Hz, 2H), 4.33 (s, 2H), 4.22-4.12 (m, 1H), 3.69 (br d, J=13.1 Hz, 1H), 3.58 (t, J=3.2 Hz, 1H), 2.94 (dt, J=3.1, 13.2 Hz, 1H), 2.17-2.08 (m, 1H), 1.67-1.56 (m, 3H), 1.50-1.47 (m, 1H), 1.44 (s, 8H), 1.25-1.20 (m, 2H), 1.14-1.08 (m, 2H), 1.05 (d, J=7.0 Hz, 3H).

5-cyclopropyl-3-(2,6-difluorophenyl)-4-((((2R,4R)-2-methylpiperidin-4-yl)oxy)methyl)isoxazole (73b). To a solution of (2R,4R)-tert-butyl 4-((5-cyclopropyl-3-(2,6-difluorophenyl)isoxazol-4-yl)methoxy)-2-methylpiperidine-1-carboxylate (73a) (190 mg, 423.63 umol) in EtOAc (2 mL) was added HCl/EtOAc (4 M, 2 mL) at 20° C. The mixture was stirred at 20° C. for 1 hour and was concentrated under reduced pressure to give 73b. MS mass calculated for [M+H]⁺(C₁₉H₂₂F₂N₂O₂) requires m/z, 349.1, LCMS found m/z, 349.1.

6-((2R,4R)-4-((5-cyclopropyl-3-(2,6-difluorophenyl)isoxazol-4-yl)methoxy)-2-methylpiperidin-1-yl)nicotinonitrile (73c). To a solution of 5-cyclopropyl-3-(2,6-difluorophenyl)-4-((((2R,4R)-2-methylpiperidin-4-yl)oxy)methyl)isoxazole (73b) (150 mg, 389.77 umol, HCl) in DMSO (2.5 mL) was added K₂CO₃ (269.34 mg, 1.95 mmol) and 6-fluoronicotinonitrile (6a) (237.95 mg, 1.95 mmol) at 20° C. and the mixture was heated to 110° C. for 16 hours. The reaction mixture was poured into H₂O (10 mL) and extracted with ethyl acetate (15 mL*3). The combined organic layer was washed with brine (10 mL*2), dried over Na₂SO₄, filtered and the filtrate was concentrated under reduced pressure to give a residue. The residue was purified by prep-TLC (SiO₂, petroleum ether:ethyl acetate=2:1) to give 73c. MS mass calculated for [M+H]⁺(C₂₅H₂₄F₂N₄O₂) requires m/z, 451.1, LCMS found m/z, 451.2; ¹H NMR (400 MHz, CHLOROFORM-d) δ=8.37 (d, J=2.1 Hz, 1H), 7.55 (dd, J=2.4, 9.1 Hz, 1H), 7.48-7.39 (m, 1H), 7.07-6.98 (m, 2H), 6.49 (d, J=9.0 Hz, 1H), 4.52-4.42 (m, 1H), 4.39 (s, 2H), 4.09 (br d, J=13.4 Hz, 1H), 3.67 (t, J=3.2 Hz, 1H), 3.12 (dt, J=2.8, 13.2 Hz, 1H), 2.14 (tt, J=5.1, 8.5 Hz, 1H), 1.78 (br s, 1H), 1.73 (br t, J=3.8 Hz, 2H), 1.66-1.58 (m, 1H), 1.29-1.22 (m, 2H), 1.14 (s, 2H), 1.13-1.09 (m, 3H).

(Z)-6-((2R,4R)-4-((5-cyclopropyl-3-(2,6-difluorophenyl)isoxazol-4-yl)methoxy)-2-methylpiperidin-1-yl)-N′-hydroxynicotinimidamide (73d). To a solution of 6-((2R,4R)-4-((5-cyclopropyl-3-(2,6-difluorophenyl)isoxazol-4-yl)methoxy)-2-methylpiperidin-1-yl)nicotinonitrile (73c) (70 mg, 155.39 umol) in EtOH (3 mL) was added hydroxylamine (10.27 mg, 155.39 umol, 0.5 mL, 50% in water) at 20° C. and the mixture was heated to 80° C. for 1 hour. The reaction mixture was poured into H₂O (10 mL) and extracted with ethyl acetate (20 mL*2). The combined organic layer was washed with brine (10 mL), dried over Na₂SO₄, filtered and the filtrate was concentrated under reduced pressure to give 73d. MS mass calculated for [M+H]⁺(C₂₅H₂₇F₂N₅O₃) requires m/z, 484.2, LCMS found m/z, 484.2.

3-(6-((2R,4R)-4-((5-cyclopropyl-3-(2,6-difluorophenyl)isoxazol-4-yl)methoxy)-2-methylpiperidin-1-yl)pyridin-3-yl)-1,2,4-oxadiazol-5(4H)-one (Compound 73). To a solution of (Z)-6-((2R,4R)-4-((5-cyclopropyl-3-(2,6-difluorophenyl)isoxazol-4-yl)methoxy)-2-methylpiperidin-1-yl)-N′-hydroxynicotinimidamide (73d) (60 mg, 124.09 umol) in EtOH (3 mL) was added diethyl carbonate (585.00 mg, 4.95 mmol) and CH₃ONa (111.73 mg, 620.46 umol, 30% in MeOH) at 20° C. and the mixture was heated to 80° C. for 30 mins. The reaction mixture was poured into H₂O (10 mL) and extracted with ethyl acetate (20 mL*2). The combined organic layer was washed with brine (10 mL), dried over Na₂SO₄, filtered and the filtrate was concentrated under reduced pressure to give a residue. The residue was purified by prep-HPLC (column: Phenomenex Gemini-NX C18 75*30 mm*3 um; mobile phase: [water (10 mM NH₄HCO₃)-ACN]; B %: 25%-55%, 8 min) to give Compound 73. MS mass calculated for [M+H]⁺(C₂₆H₂₅F₂N₅O₄) requires m/z, 510.1, LCMS found m/z, 510.1; ¹H NMR (400 MHz, CHLOROFORM-d) δ=8.49 (d, J=2.0 Hz, 1H), 7.76 (dd, J=2.1, 9.0 Hz, 1H), 7.50-7.38 (m, 1H), 7.03 (br t, J=7.8 Hz, 2H), 6.58 (br d, J=9.3 Hz, 1H), 4.48 (br s, 1H), 4.40 (s, 2H), 4.10 (br d, J=13.3 Hz, 1H), 3.67 (br s, 1H), 3.18-3.06 (m, 1H), 2.20-2.09 (m, 1H), 1.81-1.70 (m, 3H), 1.68-1.57 (m, 1H), 1.32-1.21 (m, 2H), 1.14 (br s, 2H), 1.12 (br s, 3H).

### Example 74

### 3-(6-(4-((5-cyclopropyl-3-(2,6-difluorophenyl)isoxazol-4-yl)methoxy)piperidin-1-yl)pyridin-3-yl)-1,2,4-oxadiazol-5(4H)-one

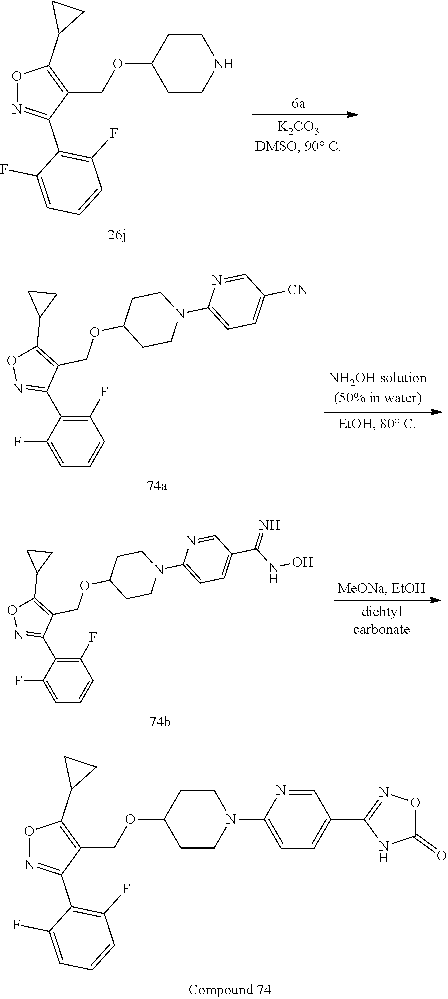 [SMILES: Fc1cccc(F)c1-c1noc(C2CC2)c1COC1CCNCC1 ; N#Cc1ccc(N2CCC(OCc3c(-c4c(F)cccc4F)noc3C3CC3)CC2)nc1 ; N=C(NO)c1ccc(N2CCC(OCc3c(-c4c(F)cccc4F)noc3C3CC3)CC2)nc1 ; O=c1[nH]c(-c2ccc(N3CCC(OCc4c(-c5c(F)cccc5F)noc4C4CC4)CC3)nc2)no1]

5-(4-((5-cyclopropyl-3-(2,6-dichlorophenyl)isoxazol-4-yl)methoxy)piperidin-1-yl)thiophene-2-carbonitrile (74a). To a solution of 5-cyclopropyl-3-(2,6-difluorophenyl)-4-((piperidin-4-yloxy)methyl)isoxazole (26j) (100 mg, 299.08 umol) and 6-fluoronicotinonitrile (6a) (43.82 mg, 358.89 umol) in CH₃CN (2 mL) was added K₂CO₃ (90.94 mg, 657.97 umol) at 20° C. The reaction mixture was stirred at 100° C. for 4h and was poured into water (6 mL) and extracted with ethyl acetate (10 mL*3). The combined organic layer was washed with brine (5 mL*2), dried over sodium sulfate, filtered and the filtrate was concentrated. The residue was purified by prep-TLC to give (74a). MS mass calculated for [M+1]⁺(C₂₄H₂₂F₂N₄O₂) requires m/z 437.2, LCMS found m/z 437.2; ¹H NMR (CHLOROFORM-d, 400 MHz): δ (ppm) 8.30 (d, J=1.8 Hz, 1H), 7.49 (dd, J=9.1, 2.4 Hz, 1H), 7.35 (tt, J=8.4, 6.4 Hz, 1H), 6.87-6.99 (m, 2H), 6.48 (d, J=9.2 Hz, 1H), 4.34 (s, 2H), 3.61-3.74 (m, 2H), 3.46 (tt, J=7.2, 3.6 Hz, 1H), 3.22-3.37 (m, 2H), 2.07 (tt, J=8.5, 5.1 Hz, 1H), 1.60-1.71 (m, 2H), 1.38-1.48 (m, 2H), 1.15-1.19 (m, 2H), 1.00-1.08 (m, 2H).

6-(4-((5-cyclopropyl-3-(2,6-difluorophenyl)isoxazol-4-yl)methoxy)piperidin-1-yl)-N-hydroxynicotinimidamide (74b). To a solution of 5-(4-((5-cyclopropyl-3-(2,6-dichlorophenyl)isoxazol-4-yl)methoxy)piperidin-1-yl)thiophene-2-carbonitrile (74a) (80 mg, 183.30 umol) in EtOH (4 mL) was added NH₂OH (1.5 mL, 50% water solution) at 20° C. The reaction mixture was heated to 80° C. for 30 min and was concentrated. The resulting mixture was poured into water (5 mL) and extracted with ethyl acetate (10 mL*2). The combined organic phase was washed with brine (5 mL), dried over sodium sulfate, filtered and the filtrate was concentrated to give crude 74b. MS mass calculated for [M+1]+(C₂₄H₂₅F₂N₅O₃) requires m/z 470.2, LCMS found m/z 470.1.

### 3-(6-(4-((5-cyclopropyl-3-(2,6-difluorophenyl)isoxazol-4-yl)methoxy)piperidin-1-yl)pyridin-3-yl)-1,2,4-oxadiazol-5(4H)-one (Compound 74)

To a solution of 6-(4-((5-cyclopropyl-3-(2,6-difluorophenyl)isoxazol-4-yl)methoxy)piperidin-1-yl)-N-hydroxynicotinimidamide (74b) (30 mg, 63.90 umol) and diethyl carbonate (377.43 mg, 3.20 mmol, 387.11 uL) in EtOH (3 mL) was added NaOMe (69.04 mg, 383.40 umol, 30% in MeOH) at 20° C. The mixture was stirred at 80° C. for 30 min and was poured into water (5 mL) and the mixture was extracted with ethyl acetate (10 mL*2). The combined organic phase was washed with brine (5 mL), dried over sodium sulfate and filtered, and the filtrate was concentrated to a residue. The residue was purified by prep-TLC (dichloromethane:methanol=10:1) to give Compound 74. MS mass calculated for [M+1]⁺(C₂₅H₂₃F₂N₅O₄) requires m/z 496.2, LCMS found m/z 496.1; ¹H NMR (CHLOROFORM-d, 400 MHz): δ (ppm) 8.43 (d, J=2.0 Hz, 1H), 7.71 (dd, J=9.2, 2.1 Hz, 1H), 7.30-7.40 (m, 1H), 6.95 (t, J=7.8 Hz, 2H), 6.57 (d, J=9.0 Hz, 1H), 4.35 (s, 2H), 3.66-3.78 (m, 2H), 3.45 (dt, J=7.2, 3.7 Hz, 1H), 3.22-3.35 (m, 2H), 2.01-2.12 (m, 1H), 1.61-1.74 (m, 2H), 1.43 (dtd, J=12.4, 8.0, 3.7 Hz, 2H), 1.16-1.22 (m, 2H), 1.00-1.08 (m, 2H).

### Example 75

### 3-(6-(4-((5-cyclopropyl-3-(2-(trifluoromethoxy)phenyl)isoxazol-4-yl)methoxy)piperidin-1-yl)pyridin-3-yl)-1,2,4-oxadiazol-5(4H)-one

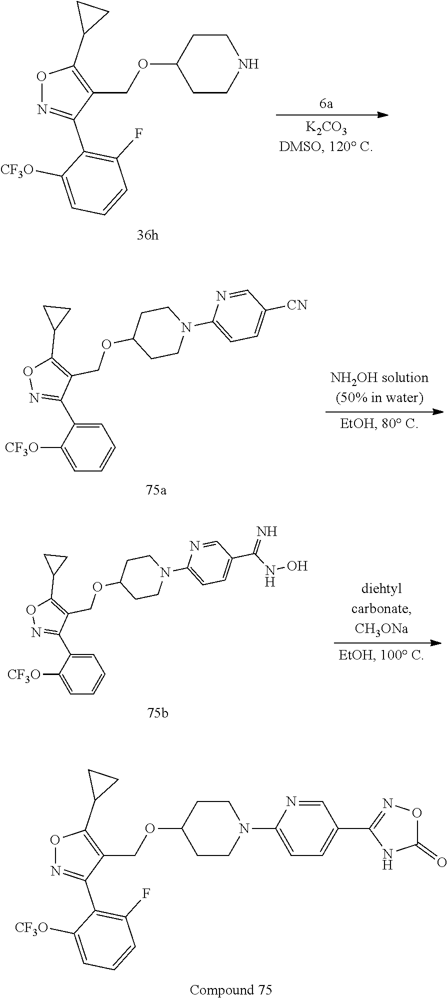

6-(4-((5-cyclopropyl-3-(2-(trifluoromethoxy)phenyl)isoxazol-4-yl)methoxy)piperidin-1-yl)nicotinonitrile (75a). To a solution 5-cyclopropyl-4-((piperidin-4-yloxy)methyl)-3-(2-(trifluoromethoxy)phenyl)isoxazole (36h) (160 mg, 418.44 umol) and 6-fluoronicotinonitrile (6a)(153.27 mg, 1.26 mmol) in DMSO (3 mL) was added K₂CO₃ (173.49 mg, 1.26 mmol) in sealed tube. The mixture was heated to 90° C. and stirred for 18 hours and was diluted with water (10 mL) and extracted with ethyl acetate (10 mL*3). The organic phase was washed with brine (5 mL*3), dried over anhydrous sodium sulfate, filtered and the filtrate was concentrated. The residue was purified by prep-TLC (SiO₂, petroleum ether:ethyl acetate=1:1) to give Compound 75a. MS mass calculated for [M+H]⁺(C₂₅H₂₃F₃N₄O₃) requires m/z, 485.2, LCMS found m/z, 485.1; ¹H NMR (400 MHz, CHLOROFORM-d) δ=8.38 (d, J=2.0 Hz, 1H), 7.65-7.55 (m, 2H), 7.54-7.48 (m, 1H), 7.45-7.33 (m, 2H), 6.57 (d, J=9.3 Hz, 1H), 4.42 (s, 2H), 3.85-3.72 (m, 2H), 3.55 (tt, J=3.5, 7.3 Hz, 1H), 3.44-3.29 (m, 2H), 2.18-2.08 (m, 1H), 1.83-1.70 (m, 2H), 1.55-1.43 (m, 2H), 1.26-1.21 (m, 2H), 1.17-1.06 (m, 2H).

(Z)-6-(4-((5-cyclopropyl-3-(2-(trifluoromethoxy)phenyl)isoxazol-4-yl)methoxy)piperidin-1-yl)-N′-hydroxynicotinimidamide (75b). To a solution of 6-(4-((5-cyclopropyl-3-(2-(trifluoromethoxy)phenyl)isoxazol-4-yl)methoxy)piperidin-1-yl)nicotinonitrile (75a) (100 mg, 206.41 umol) in EtOH (1 mL) was added hydroxylamine (0.5 mL, 50% purity). The mixture was heated to 80° C. and stirred for 2 hours in a sealed tube and was concentrated under reduced pressure. The residue was diluted with water (5 mL) and extracted with dichloromethane (10 ml*2). The combined organic phase was washed with brine (10 mL), dried over anhydrous Sodium sulfate, filtered and concentrated to give Compound 75b. MS mass calculated for [M+H]⁺(C₂₅H₂₆F₃N₅O₄) requires m/z, 518.2, LCMS found m/z, 518.1; ¹H NMR (400 MHz, CHLOROFORM-d) δ=8.38 (d, J=2.0 Hz, 1H), 7.70 (dd, J=2.4, 8.8 Hz, 1H), 7.57 (dd, J=1.7, 8.1 Hz, 1H), 7.54-7.47 (m, 1H), 7.42-7.34 (m, 2H), 6.61 (d, J=8.8 Hz, 1H), 4.77 (br s, 2H), 4.41 (s, 2H), 3.90-3.79 (m, 2H), 3.51 (tt, J=3.9, 7.8 Hz, 1H), 3.23 (ddd, J=3.4, 9.0, 13.0 Hz, 2H), 2.15 (tt, J=5.1, 8.4 Hz, 1H), 1.84-1.75 (m, 2H), 1.51 (tdd, J=4.2, 8.6, 12.8 Hz, 2H), 1.26-1.21 (m, 2H), 1.16-1.07 (m, 2H).

3-(6-(4-((5-cyclopropyl-3-(2-(trifluoromethoxy)phenyl)isoxazol-4-yl)methoxy)piperidin-1-yl)pyridin-3-yl)-1,2,4-oxadiazol-5(4H)-one (Compound 75). To a solution of (Z)-6-(4-((5-cyclopropyl-3-(2-(trifluoromethoxy)phenyl)isoxazol-4-yl)methoxy)piperidin-1-yl)-N′-hydroxynicotinimidamide (75b) (90 mg, 173.91 umol) in EtOH (1.5 mL) was added NaOMe (187.91 mg, 1.04 mmol, 30% in MeOH) and diethyl carbonate (513.61 mg, 4.35 mmol, 526.78 uL) in a sealed tube. The mixture was heated to 100° C. and stirred for 1 hour and concentrated under reduced pressure. The residue was diluted with water (10 mL) and extracted with ethyl acetate (15 mL*2). The combined organic phase was washed with brine (10 mL), dried over anhydrous sodium sulfate, filtered and the filtrate was concentrated. The residue was purified by prep-HPLC (neutral condition; column: Phenomenex Gemini-NX 80*40 mm*3 um; mobile phase: [water (10 mM NH₄HCO₃)-ACN]; B %: 15%-45%, 8 min) to give Compound 75. MS mass calculated for [M+H]⁺(C₂₆H₂₄F₃N₅O₅) requires m/z, 544.2, LCMS found m/z, 544.1; ¹H NMR (400 MHz, CHLOROFORM-d) δ=8.49 (d, J=2.4 Hz, 1H), 7.81-7.74 (m, 1H), 7.61-7.49 (m, 2H), 7.44-7.35 (m, 2H), 6.66 (d, J=9.3 Hz, 1H), 4.42 (s, 2H), 3.89-3.78 (m, 2H), 3.55 (td, J=3.7, 7.3 Hz, 1H), 3.42-3.31 (m, 2H), 2.21-2.09 (m, 1H), 1.86-1.71 (m, 2H), 1.60-1.46 (m, 2H), 1.29-1.18 (m, 2H), 1.17-1.06 (m, 2H).

### Example 76

### 3-(6-((2R,4R)-4-((5-cyclopropyl-3-(2-(trifluoromethoxy)phenyl)isoxazol-4-yl)methoxy)-2-methylpiperidin-1-yl)pyridin-3-yl)-1,2,4-oxadiazol-5(4H)-one

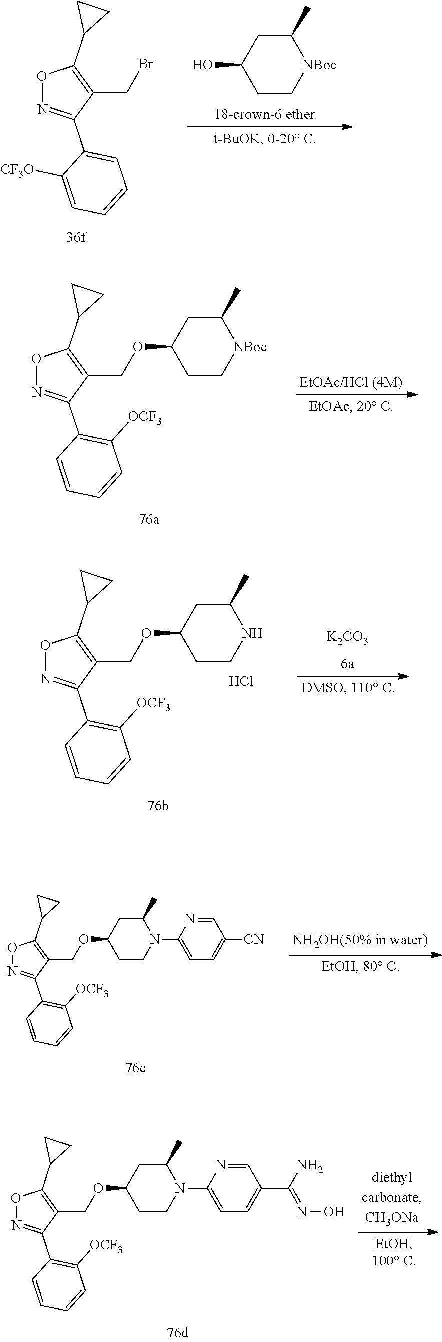

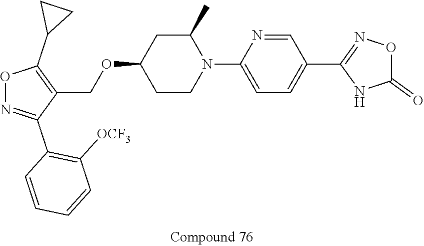 [SMILES: C[C@@H]1C[C@H](OCc2c(-c3ccccc3OC(F)(F)F)noc2C2CC2)CCN1c1ccc(-c2noc(=O)[nH]2)cn1]

(2R,4R)-tert-butyl 4-((5-cyclopropyl-3-(2-(trifluoromethoxy)phenyl)isoxazol-4-yl)methoxy)-2-methylpiperidine-1-carboxylate (76a). To a solution of 18-CROWN-6 (164.22 mg, 621.30 umol) and (2R,4R)-tert-butyl 4-hydroxy-2-methylpiperidine-1-carboxylate (98.09 mg, 455.62 umol) in THE (8 mL) was added t-BuOK (1 M in THF, 621.30 uL) at 0° C. The mixture was stirred at 20° C. for 0.5 hour. Then 4-(bromomethyl)-5-cyclopropyl-3-(2-(trifluoromethoxy)phenyl)isoxazole (36f) (150 mg, 414.20 umol) was added to the mixture and the mixture was stirred at 20° C. for 3.5 hours. The reaction mixture was poured into water (5 mL) and extracted with ethyl acetate (5 mL*4). The combined organic layer was washed with brine (20 mL), dried over sodium sulfate, filtered and the filtrate was concentrated to give the residue, which was purified by prep-TLC (petroleum ether:ethyl acetate=3:1) to give 76a. MS mass calculated for [M+H]⁺(C₂₅H₃₁F₃N₂O₅) requires m/z, 497.2, LCMS found m/z, 497.3; ¹H NMR (CHLOROFORM-d, 400 MHz): δ=7.57-7.49 (m, 2H), 7.41-7.36 (m, 2H), 4.33 (s, 2H), 4.21-4.15 (m, 1H), 3.75-3.68 (m, 1H), 3.59 (t, J=3.2 Hz, 1H), 3.50 (d, J=5.3 Hz, 1H), 2.96 (dt, J=2.9, 13.2 Hz, 1H), 2.16-2.09 (m, 1H), 1.64-1.59 (m, 2H), 1.44 (s, 9H), 1.28-1.25 (m, 1H), 1.23 (dd, J=2.3, 4.9 Hz, 2H), 1.11 (td, J=3.3, 8.3 Hz, 2H), 1.06 (d, J=7.0 Hz, 3H).

5-cyclopropyl-4-((((2R,4R)-2-methylpiperidin-4-yl)oxy)methyl)-3-(2-(trifluoromethoxy)phenyl)isoxazole (76b). To a solution of (2R,4R)-tert-butyl 4-((5-cyclopropyl-3-(2-(trifluoromethoxy)phenyl)isoxazol-4-yl)methoxy)-2-methylpiperidine-1-carboxylate (76a) (140 mg, 281.96 umol) in ethyl acetate (2 mL) was added ethyl acetate/HCl (15 mL, 4M) at 20° C. and the mixture was stirred for 4 hours. The reaction mixture was concentrated to give 76b.

6-((2R,4R)-4-((5-cyclopropyl-3-(2-(trifluoromethoxy)phenyl)isoxazol-4-yl)methoxy)-2-methylpiperidin-1-yl)nicotinonitrile (76c). To a solution of 5-cyclopropyl-4-((((2R,4R)-2-methylpiperidin-4-yl)oxy)methyl)-3-(2-(trifluoromethoxy)phenyl)isoxazole hydrochloride (76b) (100 mg, 231.02 umol) and 6-fluoronicotinonitrile (6a) (141.04 mg, 1.16 mmol) in DMSO (4 mL) was added K₂CO₃ (191.58 mg, 1.39 mmol) at 20° C. The mixture was stirred at 110° C. for 16 hours and water (5 mL) and ethyl acetate (5 mL) were added to the reaction mixture. The phases were separated and the aqueous phase was extracted with ethyl acetate (5 mL*4). The combined organic phase was washed with brine (20 mL*2), dried with anhydrous Na₂SO₄, filtered and the filtrate was concentrated under reduced pressure. The residue was purified by prep-TLC (petroleum ether:ethyl acetate=2:1, R_{f}=0.50) to give 76c. MS mass calculated for [M+H]⁺(C₂₆H₂₅F₃N₄O₃) requires m/z, 499.2, LCMS found m/z, 499.3; ¹H NMR (CHLOROFORM-d, 400 MHz): δ=8.37 (d, J=1.8 Hz, 1H), 7.58-7.53 (m, 2H), 7.53-7.49 (m, 1H), 7.41-7.36 (m, 2H), 6.49 (d, J=8.9 Hz, 1H), 4.50-4.42 (m, 1H), 4.40 (d, J=1.3 Hz, 2H), 4.10 (br d, J=16.1 Hz, 1H), 3.68 (t, J=3.4 Hz, 1H), 3.13 (dt, J=3.0, 13.2 Hz, 1H), 2.13 (tt, J=5.0, 8.5 Hz, 1H), 1.79 (br dd, J=2.8, 13.9 Hz, 1H), 1.74 (t, J=4.0 Hz, 2H), 1.66-1.56 (m, 1H), 1.27-1.22 (m, 2H), 1.14-1.09 (m, 5H).

(Z)-6-((2R,4R)-4-((5-cyclopropyl-3-(2-(trifluoromethoxy)phenyl)isoxazol-4-yl)methoxy)-2-methylpiperidin-1-yl)-N′-hydroxynicotinimidamide (76d). To a solution of 6-((2R,4R)-4-((5-cyclopropyl-3-(2-(trifluoromethoxy)phenyl)isoxazol-4-yl)methoxy)-2-methylpiperidin-1-yl)nicotinonitrile (76c) (105 mg, 210.63 umol) in ethanol (3 mL) was added hydroxylamine (41.74 mg, 631.90 umol, 1 mL, 50% in water) at 20° C. The mixture was stirred at 80° C. for 1 hour. Water (5 mL) and ethyl acetate (5 mL) were added into the reaction mixture and the phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (Dichloromethane:Methanol=10:1, R_{f}=0.13) to give 76d. ¹H NMR (CHLOROFORM-d, 400 MHz): δ=8.37 (d, J=2.4 Hz, 1H), 7.68 (dd, J=2.4, 9.0 Hz, 1H), 7.60-7.48 (m, 2H), 7.42-7.35 (m, 2H), 6.53 (d, J=9.2 Hz, 1H), 4.76 (br s, 2H), 4.46-4.40 (m, 1H), 4.39 (s, 2H), 4.01 (br d, J=13.2 Hz, 1H), 3.67 (br t, J=3.4 Hz, 1H), 3.11 (dt, J=2.7, 13.0 Hz, 1H), 2.18-2.09 (m, 1H), 1.82-1.72 (m, 3H), 1.71-1.64 (m, 1H), 1.24 (td, J=4.7, 7.1 Hz, 2H), 1.14-1.08 (m, 5H).

3-(6-((2R,4R)-4-((5-cyclopropyl-3-(2-(trifluoromethoxy)phenyl)isoxazol-4-yl)methoxy)-2-methylpiperidin-1-yl)pyridin-3-yl)-1,2,4-oxadiazol-5(4H)-one (Compound 76). To a solution of (Z)-6-((2R,4R)-4-((5-cyclopropyl-3-(2-(trifluoromethoxy)phenyl)isoxazol-4-yl)methoxy)-2-methylpiperidin-1-yl)-N′-hydroxynicotinimidamide (76d) (100 mg, 188.14 umol) and diethyl carbonate (682.50 mg, 5.78 mmol, 0.7 mL) in ethanol (3 mL) was added NaOMe (203.26 mg, 1.13 mmol, 0.3 mL, 30% in MeOH) at 20° C. The mixture was stirred at 100° C. for 4 hours and was concentrated under reduced pressure to remove solvent. Water (5 mL) and ethyl acetate (5 mL) were added to the reaction mixture and the phases were separated. The aqueous phase was extracted with ethyl acetate (5 mL*4). The combined organic phase was washed with brine (20 mL*2), dried with anhydrous Na₂SO₄, filtered and concentrated under reduced pressure to a residue which was purified by prep-HPLC (neutral condition: column: Waters Xbridge BEH C18 100*25 mm*5 um; mobile phase: [water(10 mM NH₄HCO₃)-ACN]; B %: 20%-55%, 10 min) to give Compound 76. MS mass calculated for [M+H]⁺(C₂₇H₂₆F₃N₅O₅) requires m/z, 558.2/559.2, LCMS found m/z, 558.1/559.1; ¹H NMR (CHLOROFORM-d, 400 MHz): δ=8.48 (d, J=2.2 Hz, 1H), 7.75 (dd, J=2.4, 9.0 Hz, 1H), 7.59-7.50 (m, 2H), 7.42-7.36 (m, 2H), 6.59 (d, J=9.3 Hz, 1H), 4.54-4.45 (m, 1H), 4.40 (d, J=1.1 Hz, 2H), 4.13 (br d, J=13.0 Hz, 1H), 3.68 (t, J=3.3 Hz, 1H), 3.15 (dt, J=2.8, 13.2 Hz, 1H), 2.14 (tt, J=5.1, 8.4 Hz, 1H), 1.84-1.74 (m, 3H), 1.68-1.58 (m, 1H), 1.27-1.22 (m, 2H), 1.15-1.09 (m, 5H).

### Example 77

### 3-(6-((1R,3R,5S)-3-((3-(2-chlorophenyl)-5-cyclopropylisoxazol-4-yl)methoxy)-8-azabicyclo[3.2.1]octan-8-yl)pyridin-3-yl)-1,2,4-oxadiazol-5(4H)-one

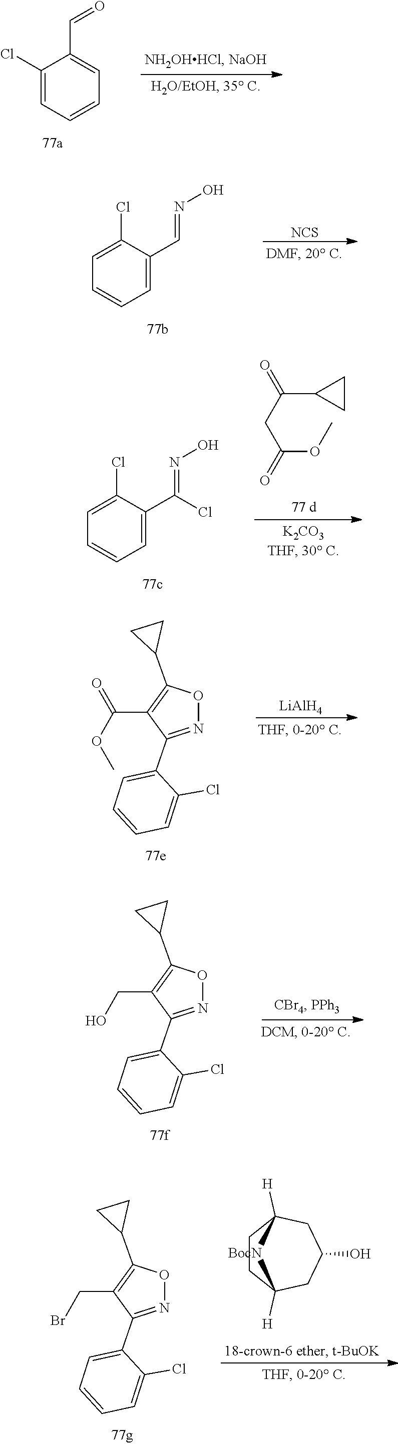

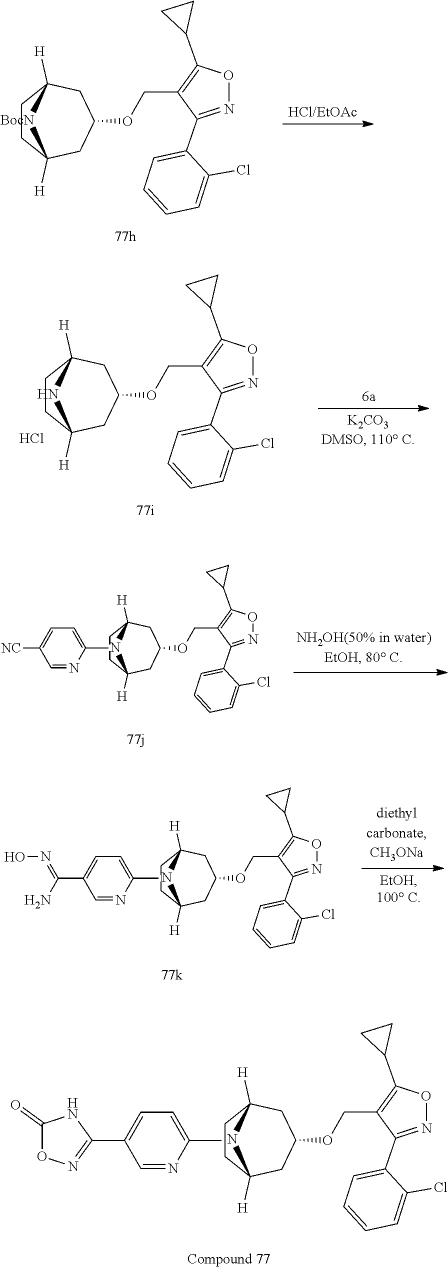

(E)-2-chlorobenzaldehyde oxime (77b). A solution of NH₂OH·HCl (543.80 mg, 7.83 mmol) and NaOH (341.45 mg, 8.54 mmol) in water (5 mL) was added dropwise to a solution of 2-chlorobenzaldehyde (77a) (1 g, 7.11 mmol, 801.09 uL) in ethanol (10 mL) at 0° C. The mixture was stirred at 35° C. for 4 hours and was concentrated to remove most of the ethanol. Water (10 mL) was added to the mixture and the resulting mixture was extracted with ethyl acetate (20 mL*3). The combined organic layer was washed with brine (20 mL), dried over sodium sulfate and concentrated to give 77b. MS mass calculated for [M+H]⁺(C₇H₆ClNO) requires m/z, 156.0/158.0, LCMS found m/z, 156.1/158.0.

(Z)-2-chloro-N-hydroxybenzimidoyl chloride (77c). To a solution of (E)-2-chlorobenzaldehyde oxime (77b) (1 g, 6.43 mmol) in DMF (6 mL) was added NCS (944.09 mg, 7.07 mmol) at 20° C. for 16 hours. This mixture of 77c in DMF was used directly in next step. MS mass calculated for [M+H]⁺(C₇H₅Cl₂NO) requires m/z, 190.0/192.0, LCMS found m/z, 190.0/191.9.

Ethyl 3-(2-chlorophenyl)-5-cyclopropylisoxazole-4-carboxylate (77e). To a solution of methyl 3-cyclopropyl-3-oxopropanoate (77d) (987.45 mg, 6.95 mmol) and K₂CO₃ (960.06 mg, 6.95 mmol) in THF (12 mL) was added (Z)-2-chloro-N-hydroxybenzimidoyl chloride (77c) in DMF (6 mL) dropwise at 0° C. The mixture was stirred at 30° C. for 8 hours and was concentrated under reduced pressure. Water (20 mL) was added to the mixture and the resulting mixture was extracted with ethyl acetate (30 mL*3). The combined organic layer was washed with brine (30 mL) and dried over sodium sulfate and concentrated. The residue was purified by flash silica gel chromatography to give 77e. MS mass calculated for [M+H]⁺(C₁₄H₁₂ClNO₃) requires m/z, 278.1/280.1, LCMS found m/z, 278.0/280.1; ¹H NMR (METHANOL-d4, 400 MHz): δ=7.50-7.46 (m, 1H), 7.44-7.39 (m, 2H), 7.38-7.33 (m, 1H), 3.70 (s, 3H), 2.89 (tt, J=5.1, 8.4 Hz, 1H), 1.41-1.36 (m, 2H), 1.30-1.23 (m, 2H).

(3-(2-chlorophenyl)-5-cyclopropylisoxazol-4-yl)methanol (77f). To a solution of ethyl 3-(2-chlorophenyl)-5-cyclopropylisoxazole-4-carboxylate (77e) (1.10 g, 3.96 mmol) in THE (15 mL) was added LiAlH₄ (450.97 mg, 11.88 mmol) at 0° C. The mixture was stirred at 0° C. for 30 minutes then was warmed to 20° C. for 1.5 hours. The reaction mixture was quenched with dropwise addition of water (0.451 mL) at 0° C., then dropwise addition of 15% NaOH solution (0.451 mL) at 0° C. The mixture was stirred at 20° C. for 10 minutes and was filtered. The filtrate was concentrated under reduced pressure. The residue was purified by flash silica gel chromatography to give 77f. MS mass calculated for [M+H]⁺(C₁₃H₁₂ClNO₂) requires m/z, 250.1/252.1, LCMS found m/z, 250.1/252.1; ¹H NMR (CHLOROFORM-d, 400 MHz): δ=7.54-7.49 (m, 1H), 7.47-7.40 (m, 2H), 7.40-7.35 (m, 1H), 4.50 (d, J=5.6 Hz, 2H), 2.20 (tt, J=5.1, 8.4 Hz, 1H), 1.51 (t, J=5.7 Hz, 1H), 1.30-1.23 (m, 2H), 1.18-1.10 (m, 2H).

4-(bromomethyl)-3-(2-chlorophenyl)-5-cyclopropylisoxazole (77g). To a solution of (3-(2-chlorophenyl)-5-cyclopropylisoxazol-4-yl) methanol (77f) (0.75 g, 3.00 mmol) in dichloromethane (20 mL) was added PPh₃ (1.58 g, 6.01 mmol) followed by CBr₄ (1.49 g, 4.51 mmol) in portions. The reaction mixture was stirred at 20° C. for 1 hour and was poured into water (15 mL) and extracted with dichloromethane (20 mL*3). The combined organic layers were washed with brine, and dried over sodium sulfate, filtered and the filtrate was concentrated to give the residue. The residue was purified by flash silica gel chromatography to give 77g. ¹HNMR (CHLOROFORM-d, 400 MHz): δ=7.55-7.50 (m, 1H), 7.50-7.42 (m, 2H), 7.42-7.37 (m, 1H), 4.47-4.31 (m, 2H), 2.18-2.09 (m, 1H), 1.32-1.25 (m, 2H), 1.23-1.16 (m, 2H).

(1R,3R,5S)-tert-butyl 3-((3-(2-chlorophenyl)-5-cyclopropylisoxazol-4-yl)methoxy)-8-azabicyclo[3.2.1]octane-8-carboxylate (77h). To a solution of 18-CROWN-6 (190.26 mg, 719.79 umol) and (1R,3R,5S)-tert-butyl 3-hydroxy-8-azabicyclo[3.2.1]octane-8-carboxylate (114.53 mg, 503.86 umol) in THF (5 mL) was added t-BuOK (1 M in THF, 719.79 uL) at 0° C. The mixture was stirred at 20° C. for 0.5 hour. 4-(bromomethyl)-3-(2-chlorophenyl)-5-cyclopropylisoxazole (77g) (150.00 mg, 479.86 umol) was added to the mixture and the mixture was stirred at 20° C. for 1.5 hours. The reaction mixture was poured into water (5 mL) and extracted with ethyl acetate (5 mL*4). The combined organic layer was washed with brine (10 mL), dried over sodium sulfate, filtered and the filtrate was concentrated to give a residue which was purified by prep-TLC (petroleum ether:ethyl acetate=3:1) to give 77h. MS mass calculated for [M+H]⁺(C₂₅H₃₁ClN₂O₄) requires m/z, 459.2/461.2, LCMS found m/z, 459.2/461.2; ¹H NMR (CHLOROFORM-d, 400 MHz): δ=7.51-7.47 (m, 1H), 7.44-7.38 (m, 2H), 7.37-7.32 (m, 1H), 4.28 (br d, J=5.9 Hz, 2H), 4.14-3.94 (m, 2H), 3.55-3.49 (m, 1H), 2.12 (tt, J=5.1, 8.5 Hz, 1H), 1.92-1.71 (m, 6H), 1.64 (br d, J=14.7 Hz, 2H), 1.44 (s, 9H), 1.25-1.21 (m, 2H), 1.13-1.08 (m, 2H).

4-(((1R,3R,5S)-8-azabicyclo[3.2.1]octan-3-yloxy)methyl)-3-(2-chlorophenyl)-5-cyclopropylisoxazole (77i). A solution of (1R,3R,5S)-tert-butyl 3-((3-(2-chlorophenyl)-5-cyclopropylisoxazol-4-yl)methoxy)-8-azabicyclo[3.2.1]octane-8-carboxylate (77h) (180 mg, 392.18 umol) in ethyl acetate/HCl (10 mL, 4M) was stirred at 20° C. for 4 hours. The reaction mixture was concentrated under reduced pressure to give 77i. MS mass calculated for [M+H]⁺(C₂₀H₂₃ClN₂O₂) requires m/z, 359.1/361.1, LCMS found m/z, 359.1/361.1.

6-((1R,3R,5S)-3-((3-(2-chlorophenyl)-5-cyclopropylisoxazol-4-yl)methoxy)-8-azabicyclo[3.2.1]octan-8-yl)nicotinonitrile (77j). To a solution of 4-(((1R,3R,5S)-8-azabicyclo[3.2.1]octan-3-yloxy)methyl)-3-(2-chlorophenyl)-5-cyclopropylisoxazole hydrochloride (77i) (80 mg, 202.37 umol) and 6-fluoronicotinonitrile (6a) (123.54 mg, 1.01 mmol) in DMSO (5 mL) was added K₂CO₃ (167.81 mg, 1.21 mmol) at 20° C. The mixture was stirred at 110° C. for 16 hours in a sealed tube. LCMS showed the reaction was finished and the reaction mixture was diluted with ethyl acetate (5 mL) and water(5 mL), and then extracted with ethyl acetate (5 mL*4), the combine organic layer was washed with brine (10 mL), dried over sodium sulfate and concentrated. The residue was purified by prep-TLC (petroleum ether:ethyl acetate=2:1, R_{f}=0.50) to give 77j. MS mass calculated for [M+H]⁺(C₂₆H₂₅ClN₄O₂) requires m/z, 461.2/463.2, LCMS found m/z, 461.2/463.2; ¹H NMR (CHLOROFORM-d, 400 MHz): δ=8.37 (d, J=1.8 Hz, 1H), 7.54 (dd, J=2.3, 8.9 Hz, 1H), 7.52-7.47 (m, 1H), 7.44-7.39 (m, 2H), 7.37-7.32 (m, 1H), 6.41 (d, J=8.9 Hz, 1H), 4.42 (br s, 2H), 4.32 (s, 2H), 3.48 (t, J=4.6 Hz, 1H), 2.12 (tt, J=5.1, 8.5 Hz, 1H), 2.01-1.92 (m, 2H), 1.90-1.82 (m, 4H), 1.75-1.68 (m, 2H), 1.26-1.22 (m, 2H), 1.15-1.09 (m, 2H).

(Z)-6-((1R,3R,5S)-3-((3-(2-chlorophenyl)-5-cyclopropylisoxazol-4-yl)methoxy)-8-azabicyclo[3.2.1]octan-8-yl)-N′-hydroxynicotinimidamide (77k). To a solution of 6-((1R,3R,5S)-3-((3-(2-chlorophenyl)-5-cyclopropylisoxazol-4-yl)methoxy)-8-azabicyclo[3.2.1]octan-8-yl)nicotinonitrile (77j) (90 mg, 195.25 umol) in ethanol (3 mL) was added hydroxylamine (38.69 mg, 585.74 umol, 1 mL, 50% in water) at 20° C. The mixture was stirred at 80° C. for 2 hours. Water (5 mL) and ethyl acetate (5 mL) were added to the reaction mixture and the phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (dichloromethane:methanol=10:1, R_{f}=0.13) to give 77k. ¹H NMR (CHLOROFORM-d, 400 MHz): δ=8.37 (d, J=2.0 Hz, 1H), 7.67 (dd, J=2.4, 8.9 Hz, 1H), 7.52-7.47 (m, 1H), 7.44-7.38 (m, 2H), 7.37-7.32 (m, 1H), 6.47 (d, J=8.8 Hz, 1H), 4.76 (br s, 2H), 4.36 (br s, 2H), 4.30 (s, 2H), 3.48-3.41 (m, 1H), 2.18-2.09 (m, 1H), 1.97-1.84 (m, 1H), 1.99-1.84 (m, 6H), 1.67 (br s, 1H), 1.63 (br s, 1H), 1.26-1.23 (m, 2H), 1.15-1.08 (m, 2H).

3-(6-((1R,3R,5S)-3-((3-(2-chlorophenyl)-5-cyclopropylisoxazol-4-yl)methoxy)-8-azabicyclo[3.2.1]octan-8-yl)pyridin-3-yl)-1,2,4-oxadiazol-5(4H)-one (Compound 77). To a solution of (Z)-6-((1R,3R,5S)-3-((3-(2-chlorophenyl)-5-cyclopropylisoxazol-4-yl)methoxy)-8-azabicyclo[3.2.1]octan-8-yl)-N′-hydroxynicotinimidamide (77k) (80 mg, 161.95 umol) and diethyl carbonate (2.54 g, 21.47 mmol, 2.60 mL) in ethanol (3 mL) was added NaOMe (174.97 mg, 971.69 umol, 0.3 mL, 30% in MeOH) at 20° C. The mixture was stirred at 100° C. for 4 hours and was concentrated under reduced pressure to remove solvent. Water (5 mL) and ethyl acetate (5 mL) were added to the reaction mixture and the phases were separated. The aqueous phase was extracted with ethyl acetate (5 mL*4). The combined organic phase was washed with brine (20 mL*2), dried with anhydrous Na₂SO₄, filtered and concentrated under reduced pressure to a residue which was purified by prep-HPLC (neutral condition: column: Waters Xbridge BEH C18 100*30 mm*10 um; mobile phase: [water(10 mM NH₄HCO₃)-ACN]; B %: 25%-50%, 8 min) to give Compound 77. MS mass calculated for [M+H]⁺(C₂₇H₂₆ClN₅O₄) requires m/z, 520.2/522.2, LCMS found m/z, 520.2/522.2; ¹H NMR (CHLOROFORM-d, 400 MHz): δ=8.45 (br s, 1H), 7.75 (dd, J=2.4, 9.0 Hz, 1H), 7.52-7.47 (m, 1H), 7.45-7.39 (m, 2H), 7.38-7.32 (m, 1H), 6.53 (d, J=9.0 Hz, 1H), 4.42 (br s, 2H), 4.32 (s, 2H), 3.48 (br s, 1H), 2.17-2.09 (m, 1H), 1.97 (br d, J=7.5 Hz, 2H), 1.93-1.82 (m, 4H), 1.71 (br d, J=14.6 Hz, 2H), 1.27-1.22 (m, 2H), 1.16-1.08 (m, 2H).

### Example 78

### 3-(6-((1R,3R,5S)-3-((5-cyclopropyl-3-(2,6-difluorophenyl)isoxazol-4-yl)methoxy)-8-azabicyclo[3.2.1]octan-8-yl)pyridin-3-yl)-1,2,4-oxadiazol-5(4H)-one

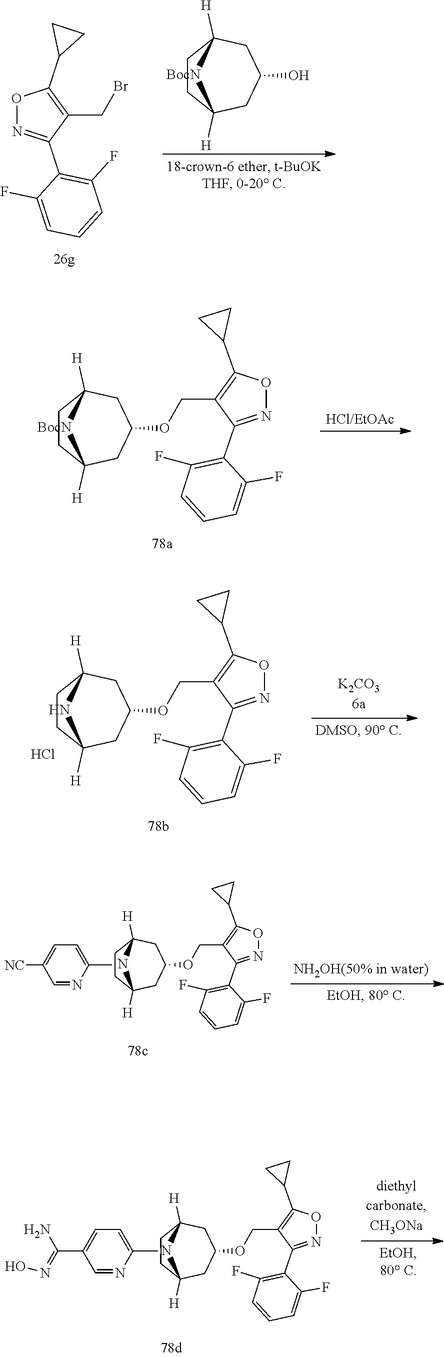

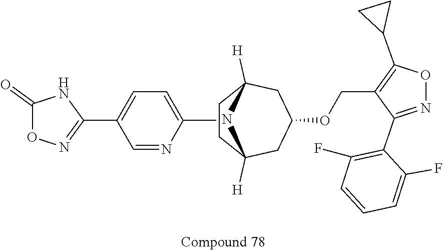

(1R,3R,5S)-tert-butyl 3-((5-cyclopropyl-3-(2,6-difluorophenyl)isoxazol-4-yl)methoxy)-8-azabicyclo[3.2.1]octane-8-carboxylate (78a). To a solution of (1R,3R,5S)-tert-butyl 3-hydroxy-8-azabicyclo[3.2.1]octane-8-carboxylate (100 mg, 439.95 umol) in THF (2 mL) was added 1,4,7,10,13,16-hexaoxacyclooctadecane (174.43 mg, 659.92 umol) and t-BuOK (1 M in THF, 659.92 uL) at 0° C. and the mixture was stirred at 20° C. for 30 mins. 4-(bromomethyl)-5-cyclopropyl-3-(2,6-difluorophenyl)isoxazole (26g) (138.20 mg, 439.95 umol) in THF (2 mL) was added dropwise to the mixture at 20° C. and the mixture was stirred at 20° C. for 1.5 hours. The reaction mixture was poured into H₂O (10 mL) and extracted with ethyl acetate (20 mL*2). The combined organic layer was washed with brine (10 mL), dried over Na₂SO₄, filtered and the filtrate was concentrated under reduced pressure to give a residue. The residue was purified by prep-TLC (SiO₂, petroleum ether:ethyl acetate=3:1) to give 78a. MS mass calculated for [M+H]⁺(C₂₅H₃₀F₂N₂O₄) requires m/z, 461.2, LCMS found m/z, 405.1, 483.1.

4-(((1R,3R,5S)-8-azabicyclo[3.2.1]octan-3-yloxy)methyl)-5-cyclopropyl-3-(2,6-difluorophenyl)isoxazole (78b). To a solution of (1R,3R,5S)-tert-butyl 3-((5-cyclopropyl-3-(2,6-difluorophenyl)isoxazol-4-yl)methoxy)-8-azabicyclo[3.2.1]octane-8-carboxylate 78a (70 mg, 152.00 umol) in EtOAc (1 mL) was added HCl/EtOAc (4 M, 2 mL) at 20° C. and the mixture was stirred at 20° C. for 1 hour. The reaction mixture was concentrated under reduced pressure to give 78b. MS mass calculated for [M+H]⁺(C₂₀H₂₂F₂N₂O₂) requires m/z, 361.1, LCMS found m/z, 361.1; ¹H NMR (400 MHz, CHLOROFORM-d) δ=9.41 (br s, 2H), 7.50-7.38 (m, 1H), 7.02 (br t, J=7.7 Hz, 2H), 4.29 (s, 2H), 3.89 (br s, 2H), 3.61 (br s, 1H), 2.34 (br s, 2H), 2.12-1.90 (m, 4H), 1.81 (br s, 2H), 1.62 (br s, 3H), 1.31-1.20 (m, 2H), 1.13 (br d, J=6.1 Hz, 2H).

6-((1R,3R,5S)-3-((5-cyclopropyl-3-(2,6-difluorophenyl)isoxazol-4-yl)methoxy)-8-azabicyclo[3.2.1]octan-8-yl)nicotinonitrile (78c). To a solution of 4-(((1R,3R,5S)-8-azabicyclo[3.2.1]octan-3-yloxy)methyl)-5-cyclopropyl-3-(2,6-difluorophenyl)isoxazole hydrochloride (78b) (50 mg, 125.99 umol) in DMSO (2 mL) was added K₂CO₃ (69.65 mg, 503.96 umol) and 6-fluoronicotinonitrile (6a) (61.53 mg, 503.96 umol) at 20° C. and the mixture was heated at 90° C. for 16 hours. The reaction mixture was poured into H₂O (10 mL) and extracted with ethyl acetate (10 mL*3). The combined organic layer was washed with brine (5 mL*2), dried over Na₂SO₄, filtered and concentrated under reduced pressure to give a residue. The residue was purified by prep-TLC (SiO₂, petroleum ether:ethyl acetate=2:1) to give 78c. MS mass calculated for [M+H]⁺(C₂₆H₂₄F₂N₄O₂) requires m/z, 463.1, LCMS found m/z, 463.2; ¹H NMR (400 MHz, CHLOROFORM-d) δ=8.37 (d, J=1.8 Hz, 1H), 7.54 (dd, J=2.3, 8.9 Hz, 1H), 7.44 (tt, J=6.4, 8.4 Hz, 1H), 7.07-6.99 (m, 2H), 6.42 (d, J=8.9 Hz, 1H), 4.53-4.33 (m, 2H), 4.33 (s, 2H), 3.52-3.46 (m, 1H), 2.12 (tt, J=5.1, 8.5 Hz, 1H), 2.02-1.92 (m, 2H), 1.92-1.81 (m, 4H), 1.74-1.66 (m, 2H), 1.28-1.21 (m, 2H), 1.16-1.08 (m, 2H).

(Z)-6-((1R,3R,5S)-3-((5-cyclopropyl-3-(2,6-difluorophenyl)isoxazol-4-yl)methoxy)-8-azabicyclo[3.2.1]octan-8-yl)-N′-hydroxynicotinimidamide (78d). To a solution of 6-((1R,3R,5S)-3-((5-cyclopropyl-3-(2,6-difluorophenyl)isoxazol-4-yl)methoxy)-8-azabicyclo[3.2.1]octan-8-yl)nicotinonitrile (78c) (40 mg, 86.49 umol) in EtOH (2 mL) was added hydroxylamine (5.71 mg, 86.49 umol, 50% in water) at 20° C. and the mixture was heated at 80° C. for 1 hour. The reaction mixture was poured into H₂O (10 mL) and extracted with ethyl acetate (20 mL*2). The combined organic layer was washed with brine (10 mL), dried over Na₂SO₄, filtered, and concentrated under reduced pressure to give 78d. MS mass calculated for [M+H]⁺(C₂₆H₂₇F₂N₅O₃) requires m/z, 496.2, LCMS found m/z, 496.2.

3-(6-((1R,3R,5S)-3-((5-cyclopropyl-3-(2,6-difluorophenyl)isoxazol-4-yl)methoxy)-8-azabicyclo[3.2.1]octan-8-yl)pyridin-3-yl)-1,2,4-oxadiazol-5(4H)-one (Compound 78). To a solution of (Z)-6-((1R,3R,5S)-3-((5-cyclopropyl-3-(2,6-difluorophenyl)isoxazol-4-yl)methoxy)-8-azabicyclo[3.2.1]octan-8-yl)-N′-hydroxynicotinimidamide (78d) (30 mg, 60.54 umol) in EtOH (1.5 mL) was added diethyl carbonate (292.50 mg, 2.48 mmol) and CH₃ONa (54.51 mg, 302.71 umol, 30% in MeOH) at 20° C. and the mixture was heated at 80° C. for 0.5 hour. The reaction mixture was poured into H₂O (10 mL) and extracted with ethyl acetate (20 mL*2). The combined organic layer was washed with brine (10 mL), dried over Na₂SO₄, filtered and concentrated under reduced pressure to give a residue. The residue was purified by prep-TLC (SiO₂, DCM:MeOH=10:1) to give Compound 78. MS mass calculated for [M+H]⁺(C₂₇H₂₅F₂N₅O₄) requires m/z, 522.1, LCMS found m/z, 522.1; ¹H NMR (400 MHz, CHLOROFORM-d) δ=8.44 (br s, 1H), 7.71 (br s, 1H), 7.49-7.38 (m, 1H), 7.02 (br t, J=7.7 Hz, 2H), 6.48 (br s, 1H), 4.50-4.32 (m, 2H), 4.31 (br s, 2H), 3.46 (br s, 1H), 2.11 (br d, J=4.9 Hz, 1H), 1.94 (br s, 2H), 1.85 (br s, 4H), 1.66 (br d, J=14.1 Hz, 2H), 1.22 (br s, 2H), 1.11 (br d, J=6.1 Hz, 2H).

### Example 79

### 3-(4-((3R,4R)-4-((5-cyclopropyl-3-(2-(trifluoromethoxy)phenyl)isoxazol-4-yl)methoxy)-3-fluoropiperidin-1-yl)phenyl)-1,2,4-oxadiazol-5(4H)-one

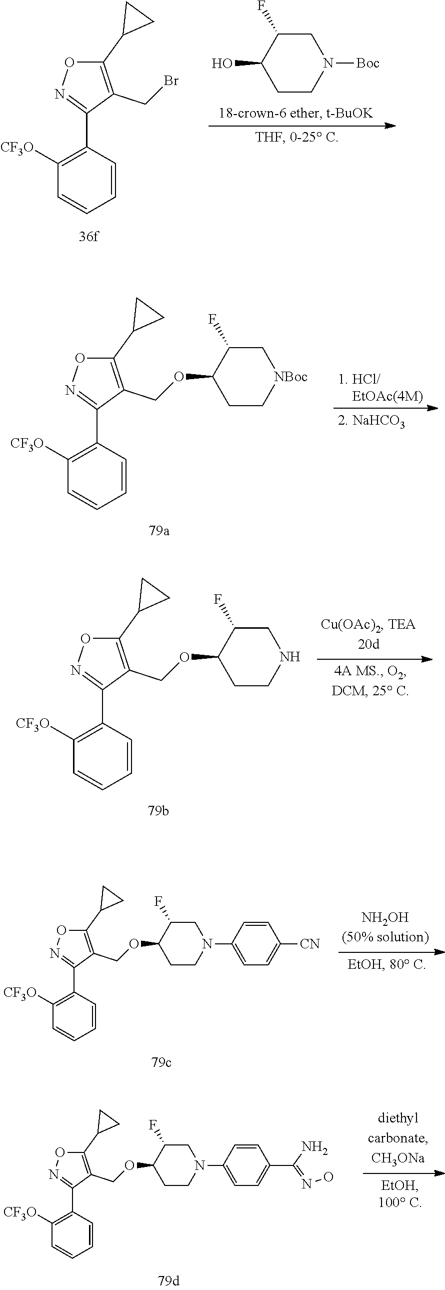

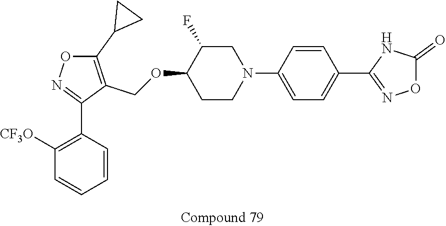

(3R,4R)-tert-butyl4-((5-cyclopropyl-3-(2-(trifluoromethoxy)phenyl)isoxazol-4-yl)methoxy)-3-fluoropiperidine-1-carboxylate (79a). To a solution of 4-(bromomethyl)-5-cyclopropyl-3-(2-(trifluoromethoxy)phenyl)isoxazole (36f) (200 mg, 552.27 umol) and (3R,4R)-tert-butyl 3-fluoro-4-hydroxypiperidine-1-carboxylate (121.09 mg, 552.27 umol) in THF (3 mL) was added 18-CROWN-6 (218.96 mg, 828.40 umol) at 15° C. The mixture was cooled to 0° C. and t-BuOK (1 M, in THF, 828.40 uL) was added dropwise and the resulting mixture was stirred for another 2 hours at 15° C. The reaction mixture was diluted with water (5 mL) and extracted with ethyl acetate (10 mL*2). The combined organic phase was washed with brine (10 mL), dried over anhydrous Sodium sulfate, filtered and concentrated. The residue was purified by prep-TLC (SiO₂, petroleum ether:ethyl acetate=3:1) to give Compound 79a. MS mass calculated for [M+H]⁺(C₂₄H₂₈F₄N₂O₅) requires m/z, 501.2, LCMS found m/z, 445.1; ¹H NMR (400 MHz, CHLOROFORM-d) δ=7.60-7.48 (m, 2H), 7.39 (t, J=7.1 Hz, 2H), 4.49 (s, 2H), 4.33 (br s, 0.5H), 4.20 (br s, 0.5H), 3.74 (br s, 1H), 3.57-3.37 (m, 2H), 3.22 (br s, 1H), 3.06 (br s, 1H), 2.17-2.08 (m, 1H), 1.76 (br d, J=6.8 Hz, 1H), 1.50-1.34 (m, 1H), 1.44 (s, 9H), 1.26-1.20 (m, 2H), 1.16-1.08 (m, 2H).

5-cyclopropyl-4-((((3R,4R)-3-fluoropiperidin-4-yl)oxy)methyl)-3-(2-(trifluoromethoxy)phenyl)isoxazole (79b). A solution of (3R,4R)-tert-butyl 4-((5-cyclopropyl-3-(2-(trifluoromethoxy)phenyl)isoxazol-4-yl)methoxy)-3-fluoropiperidine-1-carboxylate (79a) (250 mg, 499.52 umol) in HCl/ethyl acetate (10 mL, 4 M) was stirred for 2 hours at 15° C. and was concentrated under reduced pressure. The residue was dissolved in ethyl acetate (20 mL) and the mixture was washed with saturated sodium bicarbonate solution (10 mL) and brine (10 mL), dried over anhydrous Sodium sulfate and filtered. The filtrate was concentrated to give 79b which was used into next step directly. MS mass calculated for [M+H]⁺(C₁₉H₂₀F₄N₂O₃) requires m/z, 401.1/402.1, LCMS found m/z, 401.1/402.1.

4-((3R,4R)-4-((5-cyclopropyl-3-(2-(trifluoromethoxy)phenyl)isoxazol-4-yl)methoxy)-3-fluoropiperidin-1-yl)benzonitrile (79c). To a solution of 5-cyclopropyl-4-((((3R,4R)-3-fluoropiperidin-4-yl)oxy)methyl)-3-(2-(trifluoromethoxy)phenyl)isoxazole (79b) (140 mg, 349.68 umol) and (4-cyanophenyl)boronic acid (20d) (102.76 mg, 699.36 umol) in dichloromethane (5 mL) was added Cu(OAc)₂ (63.51 mg, 349.68 umol), TEA (70.77 mg, 699.36 umol, 97.34 uL) and molecular sieves 4 A (20 mg). The suspension was degassed and purged with O₂₃ times then stirred for 18 hours at 25° C. The reaction mixture was filtered through a Celite pad and the pad was rinsed with dichloromethane (15 mL). The combined organic phase was washed with water (10 mL) and brine (10 mL), dried over anhydrous sodium sulfate and filtered. The filtrate was concentrated to a residue. The residue was purified by prep-TLC (SiO₂, petroleum ether:ethyl acetate=3:1) to afford Compound 79c. MS mass calculated for [M+H]⁺(C₂₆H₂₃F₄N₃O₃) requires m/z, 502.2, LCMS found m/z, 502.1; ¹H NMR (400 MHz, CHLOROFORM-d) δ=7.62-7.44 (m, 3H), 7.40 (br d, J=7.3 Hz, 2H), 7.26-7.16 (m, 1H), 6.83 (br d, J=8.3 Hz, 2H), 4.68-4.51 (m, 2H), 4.37 (br s, 1H), 3.73-3.58 (m, 1H), 3.54 (br s, 1H), 3.40 (br s, 1H), 3.20 (td, J=6.8, 13.3 Hz, 1H), 3.03 (br d, J=9.8 Hz, 1H), 2.14 (br s, 1H), 1.89 (br s, 1H), 1.55 (br s, 1H), 1.24 (br s, 2H), 1.13 (br d, J=7.3 Hz, 2H).

(Z)-4-((3R,4R)-4-((5-cyclopropyl-3-(2-(trifluoromethoxy)phenyl)isoxazol-4-yl)methoxy)-3-fluoropiperidin-1-yl)-N′-hydroxybenzimidamide (79d). To a solution of 4-((3R,4R)-4-((5-cyclopropyl-3-(2-(trifluoromethoxy)phenyl)isoxazol-4-yl)methoxy)-3-fluoropiperidin-1-yl)benzonitrile (79c) (100 mg, 199.41 umol) in EtOH (2 mL) was added hydroxylamine (19.94 umol, 1 mL, 50% in water). The mixture was heated to 80° C. and stirred for 2 hours and was concentrated under reduced pressure to a residue. The residue was diluted with water (10 mL) and extracted with ethyl acetate (10 mL*2). The combined organic phase was washed with brine (5 mL), dried over anhydrous sodium sulfate and filtered. The filtrate was concentrated to give Compound 79d which was used into next step directly. MS mass calculated for [M+H]⁺(C₂₆H₂₆F₄N₄O₄) requires m/z, 535.2, LCMS found m/z, 535.1; ¹H NMR (400 MHz, CHLOROFORM-d) δ=7.59 (dd, J=1.5, 7.8 Hz, 1H), 7.55-7.44 (m, 3H), 7.43-7.31 (m, 2H), 6.87 (d, J=8.8 Hz, 2H), 4.80 (br s, 2H), 4.64-4.47 (m, 2H), 4.42 (dt, J=4.4, 7.8 Hz, 1H), 3.71 (dt, J=3.7, 13.1 Hz, 1H), 3.54-3.37 (m, 2H), 3.05-2.91 (m, 1H), 2.90-2.76 (m, 1H), 2.16 (dt, J=4.2, 8.9 Hz, 1H), 1.90 (dt, J=4.2, 8.7 Hz, 1H), 1.65-1.51 (m, 1H), 1.31-1.21 (m, 2H), 1.17-1.07 (m, 2H).

3-(4-((3R,4R)-4-((5-cyclopropyl-3-(2-(trifluoromethoxy)phenyl)isoxazol-4-yl)methoxy)-3-fluoropiperidin-1-yl)phenyl)-1,2,4-oxadiazol-5(4H)-one (Compound 79). To a solution (Z)-4-((3R,4R)-4-((5-cyclopropyl-3-(2-(trifluoromethoxy)phenyl)isoxazol-4-yl)methoxy)-3-fluoropiperidin-1-yl)-N′-hydroxybenzimidamide (79d) (100 mg, 187.09 umol) in EtOH (2 mL) was added NaOMe (202.15 mg, 1.12 mmol, 30% in MeOH) and diethyl carbonate (1.33 g, 11.23 mmol, 1.36 mL) in a sealed tube. The mixture was heated to 100° C. and stirred for 1 hour. The reaction mixture was concentrated under reduced pressure and was diluted with water (10 mL). The mixture was extracted with ethyl acetate (20 mL*2). The combined organic phase was washed with brine (10 mL), dried over anhydrous Sodium sulfate, filtered and concentrated. The residue was purified by prep-HPLC (neutral condition; column: Phenomenex Gemini-NX C18 75*30 mm*3 um; mobile phase: [water (10 mM NH₄HCO₃)-ACN]; B %: 31%-51%, 6 min) to give Compound 79 (55 mg, 97.15 umol, 51.92% yield, 99% purity) as a light yellow solid. MS mass calculated for [M+H]⁺(C₂₇H₂₄F₄N₄O₅) requires m/z, 561.2, LCMS found m/z, 561.0; ¹H NMR (400 MHz, CHLOROFORM-d) δ=7.63 (d, J=8.8 Hz, 2H), 7.60-7.48 (m, 2H), 7.44-7.35 (m, 2H), 6.92 (d, J=8.8 Hz, 2H), 4.60-4.47 (m, 2H), 4.60-4.35 (m, 1H), 3.76-3.62 (m, 1H), 3.60-3.49 (m, 1H), 3.44 (br d, J=13.5 Hz, 1H), 3.24-3.11 (m, 1H), 3.07-2.95 (m, 1H), 2.21-2.08 (m, 1H), 1.97-1.85 (m, 1H), 1.57 (tdd, J=4.4, 9.0, 13.4 Hz, 1H), 1.28-1.22 (m, 2H), 1.17-1.10 (m, 2H).

### Example 80

### 3-(4-((3S,4S)-4-((5-cyclopropyl-3-(2-(trifluoromethoxy)phenyl)isoxazol-4-yl)methoxy)-3-fluoropiperidin-1-yl)phenyl)-1,2,4-oxadiazol-5(4H)-one

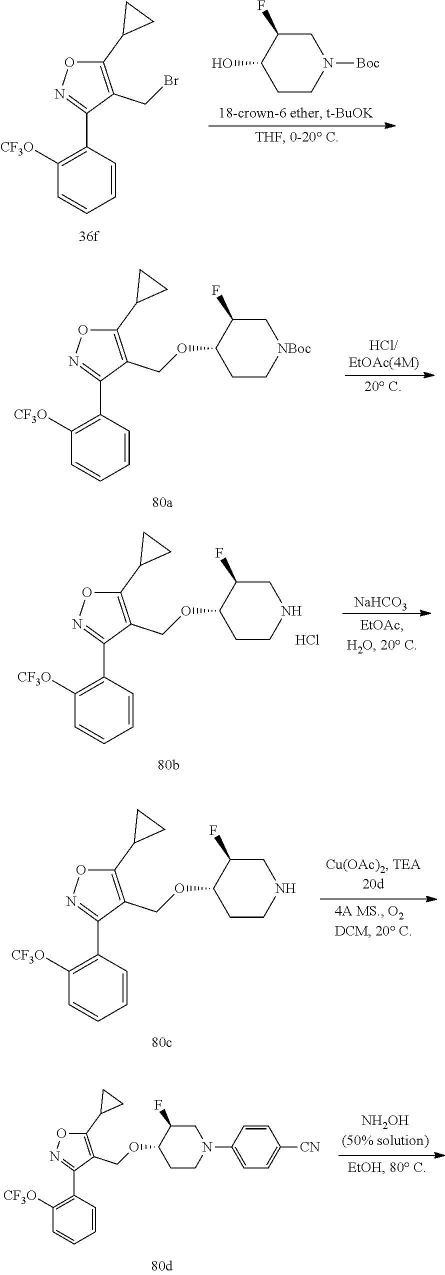

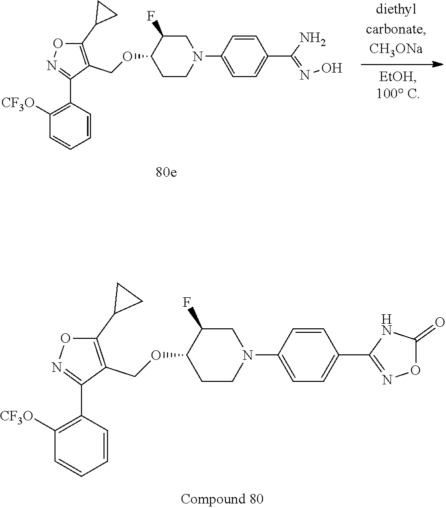

(3S,4S)-tert-butyl 4-((5-cyclopropyl-3-(2-(trifluoromethoxy)phenyl)isoxazol-4-yl)methoxy)-3-fluoropiperidine-1-carboxylate (80a). To a solution of 18-CROWN-6 (109.48 mg, 414.20 umol) and (3S,4S)-tert-butyl 3-fluoro-4-hydroxypiperidine-1-carboxylate (66.60 mg, 303.75 umol) in THF (2 mL) was added t-BuOK (1 M in THF, 414.20 uL) at 0° C. and the mixture was stirred at 20° C. for 0.5 hour. Then 4-(bromomethyl)-5-cyclopropyl-3-(2-fluorophenyl)isoxazole (36f) (100 mg, 276.14 umol) was added and the mixture was stirred at 20° C. for 1.5 hours. The reaction mixture was poured into water (5 mL) and extracted with ethyl acetate (5 mL*4). The combined organic layer was washed with brine (20 mL), dried over sodium sulfate, filtered and the filtrate was concentrated to give a residue which was purified by prep-TLC (Petroleum ether:ethyl acetate=3:1) to give 80a. ¹H NMR (CHLOROFORM-d, 400 MHz): δ=7.58-7.50 (m, 1H), 7.58-7.50 (m, 1H), 7.40 (t, J=7.3 Hz, 2H), 4.54-4.46 (m, 2H), 4.39-4.16 (m, 1H), 3.73 (br s, 1H), 3.55-3.41 (m, 2H), 3.32-3.00 (m, 2H), 2.18-2.07 (m, 1H), 1.74 (br s, 1H), 1.45 (s, 10H), 1.27-1.22 (m, 2H), 1.15-1.09 (m, 2H).

5-cyclopropyl-4-((((3S,4S)-3-fluoropiperidin-4-yl)oxy)methyl)-3-(2-(trifluoromethoxy)phenyl)isoxazole (80b). To a solution of (3S,4S)-tert-butyl 4-((5-cyclopropyl-3-(2-(trifluoromethoxy)phenyl)isoxazol-4-yl)methoxy)-3-fluoropiperidine-1-carboxylate (80a) (110 mg, 219.79 umol) in ethyl acetate (2 mL) was added HCl/EtOAc (15 mL, 4M) at 20° C. and the mixture was stirred for 4 hours. The reaction mixture was concentrated to give 80b. ¹H NMR (CHLOROFORM-d, 400 MHz): δ=7.66-7.38 (m, 4H), 4.52-4.40 (m, 2H), 3.62 (br s, 1H), 3.27 (br s, 1H), 3.07 (br s, 1H), 2.88 (br s, 1H), 2.14 (br s, 1H), 2.06 (br d, J=19.1 Hz, 2H), 1.77-1.53 (m, 2H), 1.24 (br s, 2H), 1.14 (br d, J=7.5 Hz, 2H).

5-cyclopropyl-4-((((3S,4S)-3-fluoropiperidin-4-yl)oxy)methyl)-3-(2-(trifluoromethoxy)phenyl)isoxazole (80c). To a solution of 5-cyclopropyl-4-((((3S,4S)-3-fluoropiperidin-4-yl)oxy)methyl)-3-(2-(trifluoromethoxy)phenyl)isoxazole hydrochloride (80b) (90 mg, 206.03 umol) in ethyl acetate (12 mL) and H₂O (1.5 mL) was added NaHCO₃ (138.47 mg, 1.65 mmol) at 20° C. and the mixture was stirred at 20° C. for 4 hours. The reaction mixture was dried over Na₂SO₄, filtered and concentrated under reduced pressure to give 80c.

4-((3S,4S)-4-((5-cyclopropyl-3-(2-(trifluoromethoxy)phenyl)isoxazol-4-yl)methoxy)-3-fluoropiperidin-1-yl)benzonitrile (80d). To a solution of 5-cyclopropyl-4-((((3S,4S)-3-fluoropiperidin-4-yl)oxy)methyl)-3-(2-(trifluoromethoxy)phenyl)isoxazole (80c) (80 mg, 199.82 umol) and (4-cyanophenyl)boronic acid (20d) (44.04 mg, 299.73 umol) in dichloromethane (10 mL) was added Cu(OAc)₂ (43.55 mg, 239.78 umol), 4 A MS (199.82 umol) and TEA (40.44 mg, 399.63 umol, 55.62 uL) under O₂ at 20° C. The suspension was degassed and purged with O₂ several times. The mixture was stirred under 02 (15 psi) at 20° C. for 16 hours and was filtered and the filter cake was washed by dichloromethane (50 mL). The combined filtrate was concentrated under reduced pressure. Water (5 mL) and ethyl acetate (5 mL) were added to the residue and the phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, petroleum ether:ethyl acetate=2:1, R_{f}=0.25) to give 80d. MS mass calculated for [M+H]⁺(C₂₆H₂₃F₄N₃O₃) requires m/z, 502.2, LCMS found m/z 502.2; ¹H NMR (400 MHz, CHLOROFORM-d) δ=7.59-7.47 (m, 4H), 7.42-7.36 (m, 2H), 6.83 (d, J=8.9 Hz, 2H), 4.56-4.50 (m, 2H), 3.71-3.60 (m, 1H), 3.59-3.49 (m, 1H), 3.45-3.37 (m, 1H), 3.24-3.15 (m, 1H), 3.04 (ddd, J=3.4, 9.2, 12.9 Hz, 1H), 2.14 (tt, J=5.1, 8.4 Hz, 1H), 1.95-1.86 (m, 1H), 1.60-1.57 (m, 1H), 1.54-1.51 (m, 1H), 1.28-1.23 (m, 2H), 1.16-1.10 (m, 2H).

(Z)-4-((3S,4S)-4-((5-cyclopropyl-3-(2-(trifluoromethoxy)phenyl)isoxazol-4-yl)methoxy)-3-fluoropiperidin-1-yl)-N′-hydroxybenzimidamide (80e). To a solution of 4-((3S,4S)-4-((5-cyclopropyl-3-(2-(trifluoromethoxy)phenyl)isoxazol-4-yl)methoxy)-3-fluoropiperidin-1-yl)benzonitrile (80d) (65 mg, 129.62 umol) in ethanol (3 mL) was added hydroxylamine (25.69 mg, 388.86 umol, 1 mL, 50% in water) at 20° C. and the mixture was stirred at 80° C. for 1 hour. Water (5 mL) and ethyl acetate (5 mL) were added to the reaction mixture and the phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (dichloromethane:methanol=10:1, R_{f}=0.13) to give 80e. ¹H NMR (CHLOROFORM-d, 400 MHz): δ=7.54-7.43 (m, 4H), 7.42-7.33 (m, 2H), 6.87 (br d, J=8.9 Hz, 2H), 4.80 (br s, 2H), 4.60-4.49 (m, 2H), 3.75-3.64 (m, 1H), 3.52-3.38 (m, 2H), 3.01-2.80 (m, 2H), 2.21-2.09 (m, 1H), 1.94-1.84 (m, 1H), 1.61-1.52 (m, 2H), 1.27-1.23 (m, 2H), 1.15-1.08 (m, 2H).

3-(4-((3S,4S)-4-((5-cyclopropyl-3-(2-(trifluoromethoxy)phenyl)isoxazol-4-yl)methoxy)-3-fluoropiperidin-1-yl)phenyl)-1,2,4-oxadiazol-5(4H)-one (Compound 80). To a solution of (Z)-4-((3S,4S)-4-((5-cyclopropyl-3-(2-(trifluoromethoxy)phenyl)isoxazol-4-yl)methoxy)-3-fluoropiperidin-1-yl)-N′-hydroxybenzimidamide (80e) (45 mg, 84.19 umol) and diethyl carbonate (305.42 mg, 2.59 mmol, 313.25 uL) in ethanol (3 mL) was added NaOMe (90.96 mg, 505.14 umol, 0.3 mL, 30% in MeOH) at 20° C. and the mixture was stirred at 100° C. for 2 hours. The reaction mixture was concentrated under reduced pressure to remove solvent. Water (5 mL) and ethyl acetate (5 mL) were added to the reaction mixture and the phases were separated. The aqueous phase was extracted with ethyl acetate (5 mL*4). The combined organic phase was washed with brine (20 mL*2), dried with anhydrous Na₂SO₄, filtered and concentrated under reduced pressure which was purified by prep-HPLC (neutral condition: column: Waters Xbridge BEH C18 100*25 mm*5 um; mobile phase: [water(10 mM NH₄HCO₃)-ACN]; B %: 25%-60%, 10 min) to give Compound 80. MS mass calculated for [M+H]⁺(C₂₇H₂₄F₄N₄O₅) requires m/z, 561.2/562.2, LCMS found m/z, 561.0/562.0; ¹H NMR (CHLOROFORM-d, 400 MHz): δ=7.65-7.50 (m, 4H), 7.42-7.37 (m, 2H), 6.92 (d, J=9.3 Hz, 2H), 4.60-4.52 (m, 2H), 4.52-4.36 (m, 1H), 3.70 (br t, J=13.1 Hz, 1H), 3.59-3.50 (m, 1H), 3.45 (br d, J=13.5 Hz, 1H), 3.23-3.14 (m, 1H), 3.03 (br t, J=9.6 Hz, 1H), 2.19-2.11 (m, 1H), 1.90 (br s, 1H), 1.62-1.53 (m, 1H), 1.28-1.23 (m, 2H), 1.16-1.10 (m, 2H).

### Example 81

### 5-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)isoxazol-3(2H)-one

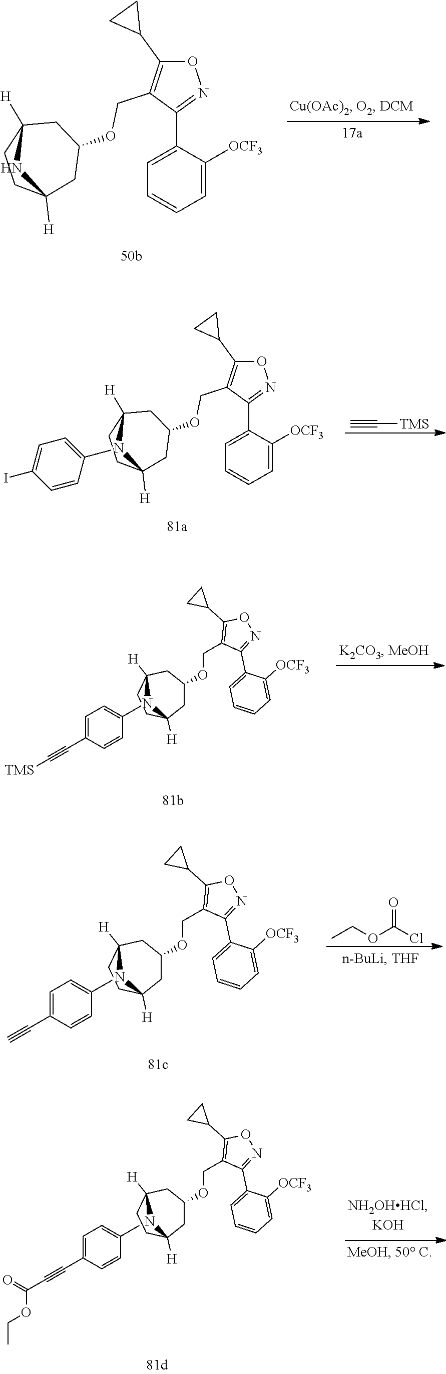

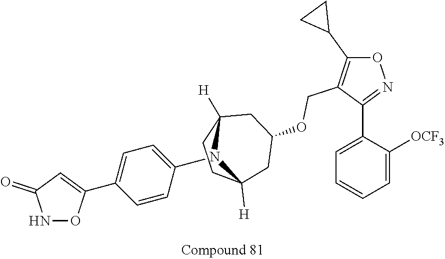

5-cyclopropyl-4-((((1R,3R,5S)-8-(4-iodophenyl)-8-azabicyclo[3.2.1]octan-3-yl)oxy)methyl)-3-(2-(trifluoromethoxy)phenyl)isoxazole (81a). To a solution of (4-iodophenyl)boronic acid (17a) (339.81 mg, 1.37 mmol) and 4-(((1R,3R,5S)-8-azabicyclo[3.2.1]octan-3-yloxy)methyl)-5-cyclopropyl-3-(2-(trifluoromethoxy)phenyl)isoxazole (50b) (280 mg, 685.58 umol) in DCM (30 mL) was added Cu(OAc)₂ (136.98 mg, 754.14 umol) under O₂ and the mixture was stirred at 30° C. for 16 hours. The reaction mixture was filtered and the filtrate was concentrated to give a residue. The residue was purified by prep-TLC (petroleum ether:ethyl acetate=4:1) to give 81a. ¹H NMR (CHLOROFORM-d, 400 MHz): δ=7.40-7.51 (m, 2H), 7.34-7.38 (m, 2H), 7.27-7.33 (m, 2H), 6.36-6.43 (m, 2H), 4.21 (s, 2H), 3.92 (br s, 2H), 3.32 (t, J=4.8 Hz, 1H), 2.00-2.09 (m, 1H), 1.73-1.93 (m, 6H), 1.45 (br s, 1H), 1.42 (s, 1H), 1.12-1.17 (m, 2H), 0.98-1.06 (m, 2H).

5-cyclopropyl-3-(2-(trifluoromethoxy)phenyl)-4-((((1R,3R,5S)-8-(4-((trimethylsilyl)ethynyl)phenyl)-8-azabicyclo[3.2.1]octan-3-yl)oxy)methyl)isoxazole (81b). A flask with a solution of 5-cyclopropyl-4-((((1R,3R,5S)-8-(4-iodophenyl)-8-azabicyclo[3.2.1]octan-3-yl)oxy)methyl)-3-(2-(trifluoromethoxy)phenyl)isoxazole (81a) (250 mg, 409.56 umol), Pd(PPh₃)₂Cl₂ (287.47 mg, 409.56 umol) and CuI (78.00 mg, 409.56 umol) was evacuated and flushed with nitrogen for 3 times. Then TEA (3 mL) and ethynyl(trimethyl)silane (321.81 mg, 3.28 mmol) were added to the mixture under nitrogen. The flask was evacuated and flushed with nitrogen again and heated to 30° C. for 16 hours. The reaction mixture was diluted with ethyl acetate (30 mL) and water (10 mL) and the mixture was stirred for 5 mins and then the phases were separated. The organic layer was washed with brine (10 mL), dried over sodium sulfate, and concentrated to give a residue. The residue was purified by prep-TLC (petroleum ether:ethyl acetate=3:1, R_{f}=0.61) to give 81b. MS mass calculated for [M+H]⁺(C₃₂H₃₅F₃N₂O₃Si) requires m/z, 581.2, LCMS found m/z, 581.2;

5-cyclopropyl-4-((((1R,3R,5S)-8-(4-ethynylphenyl)-8-azabicyclo[3.2.1]octan-3-yl)oxy)methyl)-3-(2-(trifluoromethoxy)phenyl)isoxazole (81c). To a solution of 5-cyclopropyl-3-(2-(trifluoromethoxy)phenyl)-4-((((1R,3R,5S)-8-(4-((trimethylsilyl)ethynyl)phenyl)-8-azabicyclo[3.2.1]octan-3-yl)oxy)methyl)isoxazole (81b) (240 mg, 413.29 umol) in MeOH (5 mL) was added K₂CO₃ (57.12 mg, 413.29 umol) at 20° C. and the reaction mixture was stirred at 20° C. for 16 hours. The reaction mixture was then concentrated to a residue. The residue was purified by prep-TLC (petroleum ether:ethyl acetate=5:1) to give 81c. MS mass calculated for [M+H]⁺(C₂₉H₂₇F₃N₂O₃) requires m/z, 509.2, LCMS found m/z, 509.2; ¹H NMR (CHLOROFORM-d, 400 MHz): δ=7.39-7.52 (m, 2H), 7.22-7.37 (m, 4H), 6.49-6.58 (m, 2H), 4.21 (s, 2H), 3.99 (br s, 2H), 3.62-3.71 (m, 1H), 2.88 (s, 1H), 2.04 (tt, J=8.4, 5.1 Hz, 1H), 1.78-1.91 (m, 6H), 1.43-1.51 (m, 2H), 1.12-1.17 (m, 2H), 0.99-1.05 (m, 2H).

Ethyl 3-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)propiolate (81d). To a solution of 5-cyclopropyl-4-((((1R,3R,5S)-8-(4-ethynylphenyl)-8-azabicyclo[3.2.1]octan-3-yl)oxy)methyl)-3-(2-(trifluoromethoxy)phenyl)isoxazole (81c) (80 mg, 157.32 umol) in THE (4 mL) was added n-BuLi (2.5 M, 188.78 uL) dropwise at −78° C. After addition was completed, the mixture was stirred at this temperature for 0.5 hr. Ethyl carbonochloridate (85.36 mg, 786.58 umol) was added dropwise at −78° C. to the mixture. The resulting mixture was stirred at 20° C. for 2 hours and was quenched with saturated NH₄Cl solution (4 mL). H₂O (10 mL) and ethyl acetate (20 mL) were added to the reaction mixture and the phases were separated. The aqueous phase was extracted with ethyl acetate (15 mL*2). The combined organic phase was washed with brine (10 mL), dried with anhydrous Na₂SO₄, filtered and concentrated under reduced pressure. The residue was purified by prep-TLC (SiO₂, petroleum ether:ethyl acetate=3:1) to give 81d. MS mass calculated for [M+H]⁺(C₃₂H₃₁F₃N₂O₅) requires m/z, 581.2, LCMS found m/z, 581.1; ¹H NMR (400 MHz, CHLOROFORM-d) δ=7.60-7.49 (m, 2H), 7.44 (d, J=8.8 Hz, 2H), 7.42-7.36 (m, 2H), 6.62 (d, J=8.8 Hz, 2H), 4.31 (s, 2H), 4.29-4.24 (m, 2H), 4.10 (br s, 2H), 3.47-3.40 (m, 1H), 2.11 (tt, J=5.0, 8.4 Hz, 1H), 1.99-1.94 (m, 2H), 1.94-1.84 (m, 4H), 1.60 (s, 2H), 1.35 (t, J=7.2 Hz, 3H), 1.28-1.20 (m, 2H), 1.16-1.08 (m, 2H).

5-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)isoxazol-3(2H)-one (Compound 81). To a solution of ethyl 3-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)propiolate (81d) (40 mg, 68.90 umol) in MeOH (2 mL) was added NH₂OH·HCl (47.88 mg, 688.95 umol) and KOH (69.58 mg, 1.24 mmol) at 20° C. and the mixture was heated to 50° C. for 16 hours. The reaction mixture was poured into H₂O (10 mL) and extracted with ethyl acetate (20 mL*2). The combined organic layer was washed with brine (10 mL), dried over Na₂SO₄, filtered and concentrated under reduced pressure to give a residue. The residue was purified by prep-HPLC (column: Phenomenex Luna 80*30 mm*3 um; mobile phase: [water (0.1% TFA)-ACN]; B %: 50%-80%, 7 min) to give Compound 81. MS mass calculated for [M+H]⁺(C₃₀H₂₈F₃N₃O₅) requires m/z, 568.2, LCMS found m/z, 568.1; ¹H NMR (400 MHz, CHLOROFORM-d) δ==7.56 (br d, J=8.2 Hz, 3H), 7.52 (br d, J=7.7 Hz, 1H), 7.44-7.35 (m, 2H), 6.72 (br d, J=7.5 Hz, 2H), 5.97 (s, 1H), 4.31 (s, 2H), 4.13 (br s, 2H), 3.43 (br s, 1H), 2.12 (br d, J=4.4 Hz, 1H), 1.96 (br d, J=8.2 Hz, 4H), 1.90 (br s, 2H), 1.58 (br d, J=14.8 Hz, 2H), 1.24 (br s, 2H), 1.12 (br d, J=7.3 Hz, 2H).

### Example 82

### 2-(6-(4-((5-cyclopropyl-3-(2,6-dichlorophenyl)isoxazol-4-yl)methoxy)piperidin-1-yl)pyridin-3-yl)-3, 5-dioxo-2,3,4,5-tetrahydro-1,2,4-triazine-6-carbonitrile

### Example 83

### 4-(6-(4-((5-cyclopropyl-3-(2,6-dichlorophenyl)isoxazol-4-yl)methoxy)piperidin-1-yl)pyridin-3-yl)-3, 5-dioxo-2,3,4,5-tetrahydro-1,2,4-triazine-6-carbonitrile

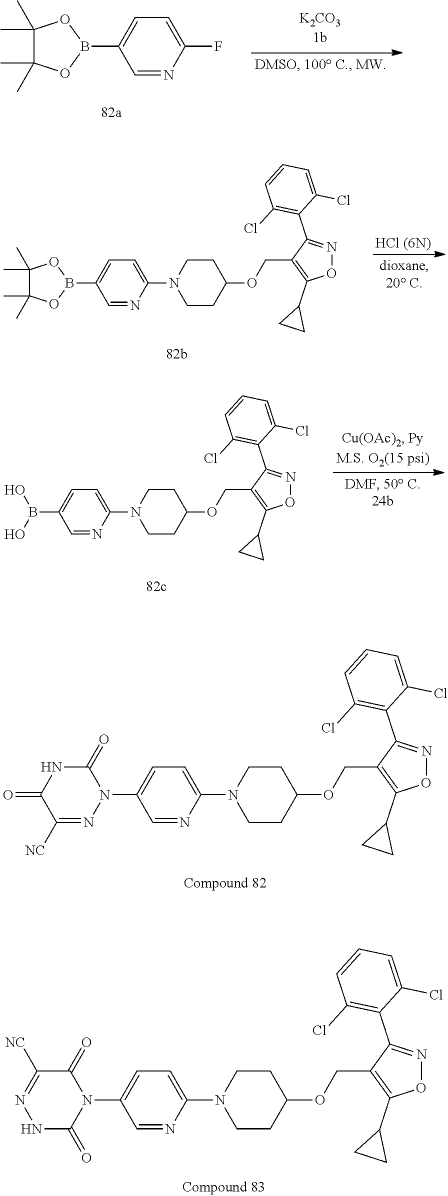 [SMILES: CC1(C)OB(c2ccc(F)nc2)OC1(C)C ; CC1(C)OB(c2ccc(N3CCC(OCc4c(-c5c(Cl)cccc5Cl)noc4C4CC4)CC3)nc2)OC1(C)C ; OB(O)c1ccc(N2CCC(OCc3c(-c4c(Cl)cccc4Cl)noc3C3CC3)CC2)nc1 ; N#Cc1nn(-c2ccc(N3CCC(OCc4c(-c5c(Cl)cccc5Cl)noc4C4CC4)CC3)nc2)c(=O)[nH]c1=O ; N#Cc1n[nH]c(=O)n(-c2ccc(N3CCC(OCc4c(-c5c(Cl)cccc5Cl)noc4C4CC4)CC3)nc2)c1=O]

5-cyclopropyl-3-(2,6-dichlorophenyl)-4-(((1-(5-(4,4,5,5-tetramethyl-1,3,2-dioxaborolan-2-yl)pyridin-2-yl)piperidin-4-yl)oxy)methyl)isoxazole (82b). To a solution of 5-cyclopropyl-3-(2,6-dichlorophenyl)-4-((piperidin-4-yloxy)methyl)isoxazole hydrochloride (1b) (500 mg, 1.24 mmol) and 2-fluoro-5-(4,4,5,5-tetramethyl-1,3,2-dioxaborolan-2-yl)pyridine (82a) (552.48 mg, 2.48 mmol) in DMSO (10 mL) was added K₂CO₃ (855.81 mg, 6.19 mmol) at 20° C. and the mixture was heated to 100° C. for 1 hour in microwave. The reaction mixture was filtered, and the filtrate was purified by prep-HPLC (column: Waters Xbridge Prep OBD C18 150*40 mm*10 um; mobile phase: [water (10 mM NH₄HCO₃)-ACN]; B %: 35%-98%, 8 min) to give 82b. MS mass calculated for [M+H]⁺(C₂₉H₃₄BCl₂N₃O₄) requires m/z, 570.2, LCMS found m/z, 569.1, 570.1; ¹H NMR (400 MHz, CHLOROFORM-d) δ=8.51 (d, J=1.2 Hz, 1H), 7.78 (dd, J=1.9, 8.6 Hz, 1H), 7.45-7.35 (m, 2H), 7.33-7.27 (m, 1H), 6.56 (d, J=8.7 Hz, 1H), 4.35 (s, 2H), 3.82-3.71 (m, 2H), 3.47 (tt, J=3.7, 7.7 Hz, 1H), 3.27-3.16 (m, 2H), 2.16 (tt, J=5.1, 8.5 Hz, 1H), 1.73 (ddd, J=3.2, 6.6, 9.4 Hz, 2H), 1.45 (dtd, J=3.9, 8.4, 12.6 Hz, 2H), 1.32 (s, 12H), 1.29-1.24 (m, 2H), 1.17-1.10 (m, 2H).

(6-(4-((5-cyclopropyl-3-(2,6-dichlorophenyl)isoxazol-4-yl)methoxy)piperidin-1-yl)pyridin-3-yl)boronic acid (82c). To a solution of 5-cyclopropyl-3-(2,6-dichlorophenyl)-4-(((1-(5-(4,4,5,5-tetramethyl-1,3,2-dioxaborolan-2-yl)pyridin-2-yl)piperidin-4-yl)oxy)methyl)isoxazole (82b) (280 mg, 490.96 umol) in dioxane (2.5 mL) was added aqueous HCl (6 M, 2.5 mL) at 20° C., and the mixture was stirred at 20° C. for 1 hour. The reaction mixture was purified by prep-HPLC (column: Waters Xbridge C18 150*50 mm*10 um; mobile phase: [water (10 mM NH₄HCO₃)-ACN]; B %: 35%-55%, 6 min) to give 82c. MS mass calculated for [M+H]⁺(C₂₃H₂₄BCl₂N₃O₄) requires m/z, 488.1, LCMS found m/z, 487.0, 488.1; ¹H NMR (400 MHz, METHANOL-d4) δ=8.45-7.78 (m, 2H), 7.55-7.48 (m, 2H), 7.47-7.39 (m, 1H), 7.01-6.64 (m, 1H), 4.40 (s, 2H), 3.57 (br s, 3H), 3.37-3.32 (m, 2H), 2.34-2.23 (m, 1H), 1.76 (br s, 2H), 1.50 (br s, 2H), 1.19 (s, 2H), 1.17 (s, 2H).

2-(6-(4-((5-cyclopropyl-3-(2,6-dichlorophenyl)isoxazol-4-yl)methoxy)piperidin-1-yl)pyridin-3-yl)-3,5-dioxo-2,3,4,5-tetrahydro-1,2,4-triazine-6-carbonitrile (Compound 82) and 4-(6-(4-((5-cyclopropyl-3-(2,6-dichlorophenyl)isoxazol-4-yl)methoxy)piperidin-1-yl)pyridin-3-yl)-3,5-dioxo-2,3,4,5-tetrahydro-1,2,4-triazine-6-carbonitrile (Compound 83). To a solution of (6-(4-((5-cyclopropyl-3-(2,6-dichlorophenyl)isoxazol-4-yl)methoxy)piperidin-1-yl)pyridin-3-yl)boronic acid (82c) (80 mg, 163.88 umol) and 3,5-dioxo-2,3,4,5-tetrahydro-1,2,4-triazine-6-carbonitrile (45.26 mg, 327.75 umol) in DMF (3 mL) were added Cu(OAc)₂ (35.72 mg, 196.65 umol), Molecular sieve 4 A (30 mg) and pyridine (25.93 mg, 327.75 umol) at 20° C. The mixture was stirred at 50° C. for 16 hours under O₂ atmosphere. The reaction mixture was poured into H₂O (10 mL) and extracted with ethyl acetate (15 mL*3). The combined organic layer was washed with brine (10 mL), dried over Na₂SO₄, filtered and concentrated under reduced pressure to give a residue. The residue was purified by prep-TLC (SiO₂, DCM:MeOH=10:1) to give Compound 82(crude) and (Compound 83) (crude).

Compound 82 (crude) was repurified by prep-HPLC (column: Waters Xbridge BEH C18 100*30 mm*10 um; mobile phase: [water (10 mM NH₄HCO₃)-ACN]; B %: 25%-55%, 8 min) to give Compound 82. MS mass calculated for [M+H]⁺(C₂₇H₂₃Cl₂N₇O₄) requires m/z, 580.1, LCMS found m/z, 580.0; ¹H NMR (400 MHz, CHLOROFORM-d) δ=8.21 (d, J=2.6 Hz, 1H), 7.49 (dd, J=2.4, 9.0 Hz, 1H), 7.42-7.36 (m, 2H), 7.33-7.28 (m, 1H), 6.60 (d, J=9.0 Hz, 1H), 4.35 (s, 2H), 3.68 (br d, J=7.3 Hz, 2H), 3.53-3.45 (m, 1H), 3.27 (br t, J=9.0 Hz, 2H), 2.20-2.11 (m, 1H), 1.71 (br d, J=3.1 Hz, 2H), 1.47 (br d, J=8.6 Hz, 2H), 1.31-1.24 (m, 2H), 1.16-1.09 (m, 2H).

(Compound 83) (crude) was repurified by prep-HPLC (column: Waters Xbridge BEH C18 100*25 mm*5 um; mobile phase: [water (10 Mm NH₄HCO₃)-ACN]; B %: 20%-55%, 10 min) to give Compound 83. MS mass calculated for [M+H]⁺(C₂₇H₂₃Cl₂N₇O₄) requires m/z, 580.1, LCMS found m/z, 580.0; ¹H NMR (400 MHz, CHLOROFORM-d) δ=8.02 (d, J=2.6 Hz, 1H), 7.43-7.38 (m, 2H), 7.34-7.30 (m, 1H), 7.29 (br d, J=6.0 Hz, 1H), 6.68 (d, J=9.0 Hz, 1H), 4.36 (s, 2H), 3.75-3.65 (m, 2H), 3.52 (td, J=3.7, 7.5 Hz, 1H), 3.34-3.24 (m, 2H), 2.20-2.12 (m, 1H), 1.80-1.70 (m, 2H), 1.54-1.44 (m, 2H), 1.31-1.25 (m, 2H), 1.18-1.11 (m, 2H).

### Example 84

### 5-(4-(4-((5-cyclopropyl-3-(2-(trifluoromethoxy)phenyl)isoxazol-4-yl)methoxy)-3,3-difluoropiperidin-1-yl)phenyl)isoxazol-3(2H)-one

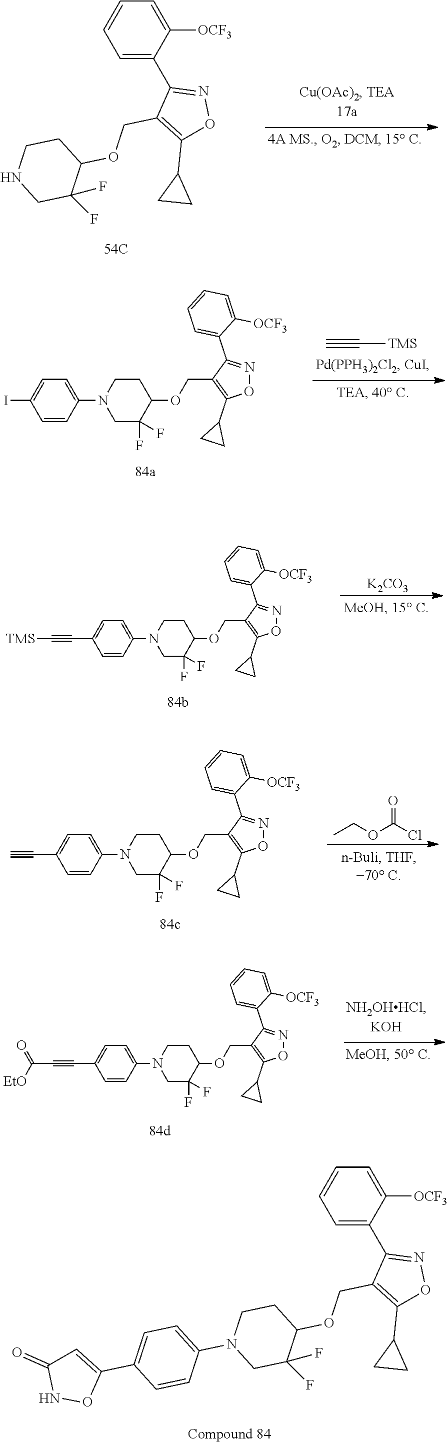

Tert-5-cyclopropyl-4-(((3,3-difluoro-1-(4-iodophenyl)piperidin-4-yl)oxy)methyl)-3-(2-(trifluoromethoxy)phenyl)isoxazole (84a). To a solution of 5-cyclopropyl-4-(((3,3-difluoropiperidin-4-yl)oxy)methyl)-3-(2-(trifluoromethoxy)phenyl)isoxazole (54c) (400 mg, 956.12 umol) and (4-iodophenyl)boronic acid (17a) (473.90 mg, 1.91 mmol) in dichloromethane (5 mL) was added Cu(OAc)₂ (173.66 mg, 956.12 umol), TEA (193.50 mg, 1.91 mmol, 266.16 uL) and Molecular sieve 4 A (20 mg). The reaction mixture was degassed and purged with O₂ three times and was stirred for 18 hours at 25° C. The reaction mixture was filtered through a Celite pad and the pad was rinsed with dichloromethane (15 mL), The combined filtrate was washed with water (10 mL) and brine (10 mL), dried over anhydrous sodium sulfate, filtered and concentrated. The residue was purified by column chromatography (SiO₂, petroleum ether:ethyl acetate=1:0 to 30:1) to give Compound 84a. MS mass calculated for [M+H]⁺(C₂₅H₂₂F₅₁N₂O₃) requires m/z, 621.2, LCMS found m/z, 621.0; ¹H NMR (400 MHz, CHLOROFORM-d) δ=7.61-7.45 (m, 4H), 7.43-7.33 (m, 2H), 6.63 (br d, J=9.3 Hz, 2H), 4.68 (d, J=11.7 Hz, 1H), 4.48 (d, J=12.2 Hz, 1H), 3.59 (br dd, J=5.1, 9.0 Hz, 1H), 3.38-3.20 (m, 2H), 3.15-2.94 (m, 2H), 2.19-2.07 (m, 1H), 1.87 (dt, J=4.6, 8.9 Hz, 1H), 1.71 (br dd, J=3.9, 9.8 Hz, 1H), 1.26-1.20 (m, 2H), 1.16-1.08 (m, 2H).

5-cyclopropyl-4-(((3,3-difluoro-1-(4-((trimethylsilyl)ethynyl)phenyl)piperidin-4-yl)oxy)methyl)-3-(2-(trifluoromethoxy)phenyl)isoxazole (84b). To a solution of 5-cyclopropyl-4-(((3,3-difluoro-1-(4-iodophenyl)piperidin-4-yl)oxy)methyl)-3-(2-(trifluoromethoxy)phenyl)isoxazole (84a) (350 mg, 564.20 umol) and ethynyl(trimethyl)silane (554.14 mg, 5.64 mmol, 781.59 uL) in TEA (5 mL) was added Pd(PPh₃)₂Cl₂ (396.01 mg, 564.20 umol) and CuI (107.45 mg, 564.20 umol). The mixture was degassed and purged with N₂ 3 times and was heated to 40° C. and stirred for 18 hours. The reaction mixture was concentrated under reduced pressure and diluted with ethyl acetate (40 mL). 3-Mercaptopropyl-functionalized silica gel (1 g) was added the mixture and the resulting mixture was stirred at 40° C. for 2 hours and was filtered through a Celite pad. The filtrate was washed with water (10 mL) and brine (10 mL), dried over anhydrous sodium sulfate, filtered and concentrated. The residue was purified by flash silica gel chromatography (ISCO®; 12 g SepaFlash® Silica Flash Column, Eluent of 0˜10% ethyl acetate/petroleum ether gradient @ 30 mL/min) to give Compound 84b. MS mass calculated for [M+H]⁺(C₃₀H₃₁F₅N₂O₃Si) requires m/z, 591.2, LCMS found m/z, 591.1; ¹H NMR (400 MHz, CHLOROFORM-d) δ=7.58-7.48 (m, 2H), 7.42-7.37 (m, 2H), 7.35 (d, J=8.9 Hz, 2H), 6.75 (d, J=8.9 Hz, 2H), 4.68 (d, J=11.7 Hz, 1H), 4.49 (d, J=11.8 Hz, 1H), 3.60 (br dd, J=4.7, 9.2 Hz, 1H), 3.50-3.26 (m, 2H), 3.21-3.11 (m, 1H), 3.11-2.98 (m, 1H), 2.13 (tt, J=5.1, 8.4 Hz, 1H), 1.87 (ddd, J=3.9, 9.4, 13.7 Hz, 1H), 1.71 (br dd, J=4.1, 9.7 Hz, 1H), 1.25 (qd, J=3.3, 5.1 Hz, 2H), 1.18-1.08 (m, 2H), 0.24 (s, 9H).

5-cyclopropyl-4-(((1-(4-ethynylphenyl)-3,3-difluoropiperidin-4-yl)oxy)methyl)-3-(2-(trifluoromethoxy)phenyl)isoxazole (84c). To a solution of 5-cyclopropyl-4-(((3,3-difluoro-1-(4-((trimethylsilyl)ethynyl)phenyl)piperidin-4-yl)oxy)methyl)-3-(2-(trifluoromethoxy)phenyl)isoxazole (84b) (300 mg, 507.91 umol) in methanol (1 mL) was added K₂CO₃ (70.20 mg, 507.91 umol) and the mixture was stirred for 3 hours at 15° C. The reaction mixture was concentrated under reduced pressure and the residue was diluted with water (10 mL) and extracted with ethyl acetate (20 mL*2). The combined organic phase was washed with brine (10 mL), dried over anhydrous sodium sulfate, filtered and concentrated. The residue was purified by prep-TLC (SiO₂, petroleum ether:ethyl acetate=3:1) to give Compound 84c. MS mass calculated for [M+H]⁺(C₂₇H₂₃F₅N₂O₃) requires m/z, 519.2, LCMS found m/z, 519.1; ¹H NMR (400 MHz, CHLOROFORM-d) δ=7.63-7.46 (m, 2H), 7.45-7.30 (m, 4H), 6.78 (br d, J=8.3 Hz, 2H), 4.68 (br d, J=11.7 Hz, 1H), 4.49 (br d, J=11.7 Hz, 1H), 3.60 (br d, J=4.4 Hz, 1H), 3.48-3.27 (m, 2H), 3.22-3.12 (m, 1H), 3.11-3.03 (m, 1H), 2.99 (s, 1H), 2.20-2.08 (m, 1H), 1.88 (br s, 1H), 1.73 (br s, 1H), 1.29-1.21 (m, 2H), 1.13 (br d, J=5.4 Hz, 2H).

Ethyl 3-(4-(4-((5-cyclopropyl-3-(2-(trifluoromethoxy)phenyl)isoxazol-4-yl)methoxy)-3,3-difluoropiperidin-1-yl)phenyl)propiolate (84d). To a solution of 5-cyclopropyl-4-(((1-(4-ethynylphenyl)-3,3-difluoropiperidin-4-yl)oxy)methyl)-3-(2-(trifluoromethoxy)phenyl)isoxazole (84c) (100 mg, 192.87 umol) in THE (2 mL) was added n-BuLi (2.5 M in hexane, 385.75 uL) dropwise at −70° C. and stirred for 30 min. Then ethyl carbonochloridate (104.66 mg, 964.37 umol, 91.80 uL) in THE (1 mL) was added dropwise to the mixture and the resulting mixture was stirred for 4 hours at this temperature, The reaction mixture was warmed to 0° C. and quenched with saturated ammonium chloride solution (5 mL) and was extracted with ethyl acetate (10 mL*2). The combined organic phase was washed with brine (10 mL), dried over anhydrous sodium sulfate, filtered and concentrated. The residue was purified by prep-TLC (SiO₂, petroleum ether:ethyl acetate=3:1) to give Compound 84d. MS mass calculated for [M+H]⁺(C₃₀H₂₇F₅N₂O₅) requires m/z, 591.2, LCMS found m/z, 591.2; ¹H NMR (400 MHz, CHLOROFORM-d) δ=7.63-7.46 (m, 2H), 7.45-7.30 (m, 4H), 6.78 (br d, J=8.3 Hz, 2H), 4.68 (br d, J=11.7 Hz, 1H), 4.49 (br d, J=11.7 Hz, 1H), 3.60 (br d, J=4.4 Hz, 1H), 3.48-3.27 (m, 2H), 3.22-3.12 (m, 1H), 3.11-3.03 (m, 1H), 2.99 (s, 1H), 2.20-2.08 (m, 1H), 1.88 (br s, 1H), 1.73 (br s, 1H), 1.29-1.21 (m, 2H), 1.13 (br d, J=5.4 Hz, 2H).

5-(4-(4-((5-cyclopropyl-3-(2-(trifluoromethoxy)phenyl)isoxazol-4-yl)methoxy)-3,3-difluoropiperidin-1-yl)phenyl)isoxazol-3(2H)-one (Compound 84). To a solution of ethyl 3-(4-(4-((5-cyclopropyl-3-(2-(trifluoromethoxy)phenyl)isoxazol-4-yl)methoxy)-3,3-difluoropiperidin-1-yl)phenyl)propiolate (84d) (30 mg, 50.80 umol) in MeOH (1 mL) was added hydroxylamine hydrochloride (35.30 mg, 508.01 umol) and KOH (51.30 mg, 914.42 umol). The mixture was heated to 50° C. and stirred for 4 hours. The reaction mixture was concentrated under reduced pressure and was diluted with water (5 mL) and extracted with dichloromethane (10 mL*2). The combined organic phase was washed with brine (10 mL), dried over anhydrous Sodium sulfate, filtered and concentrated. The residue was purified by prep-HPLC (neutral condition; column: Waters Xbridge BEH C18 100*30 mm*10 um; mobile phase: [water (10 mM NH₄HCO₃)-ACN]; B %: 30%-60%, 8 min) and lyophilized to give Compound 84. MS mass calculated for [M+H]⁺(C₂₈H₂₄F₅N₃O₅) requires m/z, 578.2, LCMS found m/z, 578.0; ¹H NMR (400 MHz, CHLOROFORM-d) δ=7.60 (br d, J=8.3 Hz, 2H), 7.58-7.46 (m, 2H), 7.40 (br d, J=6.8 Hz, 2H), 6.89 (br d, J=8.8 Hz, 2H), 6.04 (s, 1H), 4.70 (br d, J=11.7 Hz, 1H), 4.50 (br d, J=11.7 Hz, 1H), 3.63 (br d, J=2.9 Hz, 1H), 3.57-3.34 (m, 2H), 3.25 (br s, 1H), 3.20-3.07 (m, 1H), 2.19-2.09 (m, 1H), 1.90 (br s, 1H), 1.74 (br s, 1H), 1.25 (br s, 2H), 1.14 (br d, J=4.9 Hz, 2H).

### Example 85

### 2-(4-(4-((5-cyclopropyl-3-(2-(trifluoromethoxy)phenyl)isoxazol-4-yl)methoxy)piperidin-1-yl)phenyl)-3,5-dioxo-2,3,4,5-tetrahydro-1,2,4-triazine-6-carbonitrile

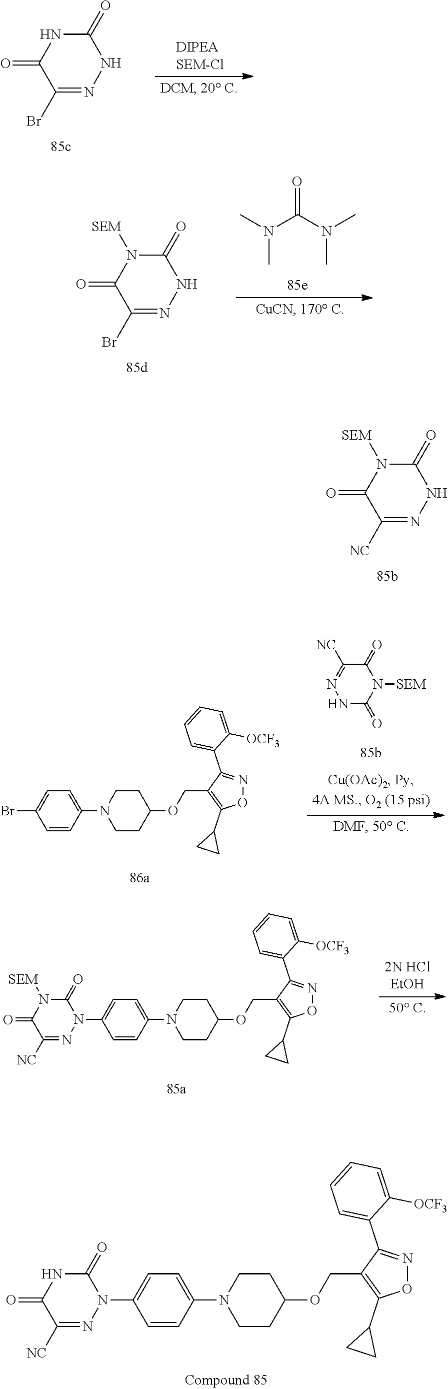

6-bromo-1,2,4-triazine-3,5(2H,4H)-dione(2). To a solution of 6-bromo-2H-1,2,4-triazine-3,5-dione (10 g, 52.09 mmol) in DCM (10 mL) was added SEM-Cl (8.68 g, 52.09 mmol, 9.22 mL) and DIEA (13.46 g, 104.18 mmol, 18.15 mL) at 20° C. The reaction was degassed and purged with N₂ 3 times, and stirred at 20° C. for 6 hr under N₂ atmosphere. The reaction mixture was concentrated under reduced pressure. Water (25 mL) and ethyl acetate (25 mL) were added into the residue and the phases were separated. The aqueous phase was extracted with ethyl acetate (25 mL*2). The combined organic phase was washed with brine (30 mL*6), dried with anhydrous Na₂SO₄, filtered and concentrated under reduced pressure. The residue was purified by flash silica gel chromatography (ISCO®; 120 g SepaFlash® Silica Flash Column, Eluent of 0-100% ethyl acetate/petroleum ethergradient @ 80 mL/min) to give 85d. ¹H NMR (400 MHz, CHLOROFORM-d) δ=10.46 (s, 1H), 5.42 (s, 2H), 3.75-3.69 (m, 2H), 1.01-0.95 (m, 2H), 0.01 (s, 9H).

3,5-dioxo-4-((2-(trimethylsilyl)ethoxy)methyl)-2,3,4,5-tetrahydro-1,2,4-triazine-6-carbonitrile (85b). To a solution of 6-bromo-4-(2-trimethylsilylethoxymethyl)-2H-1,2,4-triazine-3,5-dione (1.00 g, 3.10 mmol) in 1,1,3,3-tetramethylurea (5.81 g, 50.00 mmol, 6 mL) was added CuCN (555.88 mg, 6.21 mmol, 1.36 mL) at 20° C. The reaction was degassed and purged with N₂ 3 times, and then the mixture was stirred at 170° C. for 8 hr. The reaction mixture was poured into water (50 mL) and then ethyl acetated (150 mL) was added, the slurry was then filtered, the filtrate was separated. The aqueous phase was extracted with ethyl acetate (50 mL*2). The combined organic phase was washed with brine (50 mL*2), dried over sodium sulfate and concentrated. The residue was purified by prep-TLC (SiO₂, dichloromethane:methanol=20:1) to give compound 85b; MS mass calculated for [M−H]⁻ (C₁₀H₁₆N₄O₃Si) requires m/z, 267.10, LCMS found m/z, 266.9; ¹H NMR (400 MHz, CHLOROFORM-d) δ=5.42-5.36 (m, 2H), 3.75-3.68 (m, 2H), 1.01-0.95 (m, 2H), 0.06-0.00 (m, 9H).

2-(4-(4-((5-cyclopropyl-3-(2-(trifluoromethoxy)phenyl)isoxazol-4-yl)methoxy)piperidin-1-yl)phenyl)-3,5-dioxo-4-((2-(trimethylsilyl)ethoxy)methyl)-2,3,4,5-tetrahydro-1,2,4-triazine-6-carbonitrile (85a). To a solution of 3,5-dioxo-4-((2-(trimethylsilyl)ethoxy)methyl)-2,3,4,5-tetrahydro-1,2,4-triazine-6-carbonitrile (85b) (32.05 mg, 119.45 umol) and 4-(((1-(4-bromophenyl)piperidin-4-yl)oxy)methyl)-5-cyclopropyl-3-(2-(trifluoromethoxy)phenyl)isoxazole (86a) (40 mg, 79.64 umol) in DMF (4 mL) was added Cu(OAc)₂ (14.46 mg, 79.64 umol), 4 A MS (10 mg), Py (12.60 mg, 159.27 umol, 12.86 uL) under O₂ at 20° C. The suspension was degassed under vacuum and purged with 02 several times. The mixture was stirred under O₂ (15 psi) at 50° C. for 16 hours. The reaction mixture was concentrated under reduced pressure to remove solvent. Water (5 mL) and ethyl acetate (5 mL) were added to the reaction mixture and the phases were separated. The aqueous phase was extracted with ethyl acetate (5 mL*4). The combined organic phase was washed with brine (20 mL*2), dried with anhydrous Na₂SO₄, filtered and the filtrate was concentrated under reduced pressure. The residue was purified by prep-TLC (SiO₂, dichloromethane:methanol=50:1, R_{f}=0.50) to give 85a. MS mass calculated for [M+H]⁺(C₃₅H₃₉F₃N₆O₆Si) requires m/z, 725.3, LCMS found m/z, 725.3; ¹H NMR (400 MHz, CHLOROFORM-d) δ=7.60-7.56 (m, 1H), 7.54-7.48 (m, 1H), 7.41-7.35 (m, 2H), 7.31 (d, J=8.9 Hz, 2H), 6.91 (br d, J=8.9 Hz, 2H), 5.46 (s, 2H), 4.41 (s, 2H), 3.78-3.72 (m, 2H), 3.49-3.39 (m, 3H), 3.03-2.94 (m, 2H), 2.18-2.11 (m, 1H), 1.83 (br s, 2H), 1.63-1.59 (m, 1H), 1.25 (td, J=2.7, 5.2 Hz, 3H), 1.15-1.09 (m, 2H), 1.02-0.96 (m, 2H), 0.03 (s, 9H).

2-(4-(4-((5-cyclopropyl-3-(2-(trifluoromethoxy)phenyl)isoxazol-4-yl)methoxy)piperidin-1-yl)phenyl)-3,5-dioxo-2,3,4,5-tetrahydro-1,2,4-triazine-6-carbonitrile (Compound 85). To a solution of 2-(4-(4-((5-cyclopropyl-3-(2-(trifluoromethoxy)phenyl)isoxazol-4-yl)methoxy)piperidin-1-yl)phenyl)-3, 5-dioxo-4-((2-(trimethylsilyl)ethoxy)methyl)-2,3,4,5-tetrahydro-1,2,4-triazine-6-carbonitrile (85a) (20 mg, 27.59 umol) in ethanol (1 mL) was added aqueous HCl (2 M, 2.00 mL) at 20° C. and the mixture was heated to 50° C. for 16 hours. The reaction mixture was concentrated under reduced pressure to remove solvent. The residue was purified by prep-HPLC (neutral condition: column: Waters Xbridge BEH C18 100*30 mm*10 um; mobile phase: [water (10 mM NH₄HCO₃)-ACN]; B %: 25%-55%, 6 min) to give Compound 85. MS mass calculated for [M+H]⁺(C₂₉H₂₅F₃N₆O₅) requires m/z, 595.2, LCMS found m/z 595.0; ¹H NMR (400 MHz, CHLOROFORM-d) δ=7.60-7.55 (m, 1H), 7.54-7.48 (m, 1H), 7.41-7.35 (m, 2H), 7.30 (s, 2H), 6.89 (d, J=9.0 Hz, 2H), 4.40 (s, 2H), 3.48-3.38 (m, 3H), 3.02-2.91 (m, 2H), 2.18-2.10 (m, 1H), 1.82 (br s, 2H), 1.64-1.53 (m, 2H), 1.26-1.22 (m, 2H), 1.15-1.07 (m, 2H).

### Example 86

### 4-(4-(4-((5-cyclopropyl-3-(2-(trifluoromethoxy)phenyl)isoxazol-4-yl)methoxy)piperidin-1-yl)phenyl)-3,5-dioxo-2,3,4,5-tetrahydro-1,2,4-triazine-6-carbonitrile

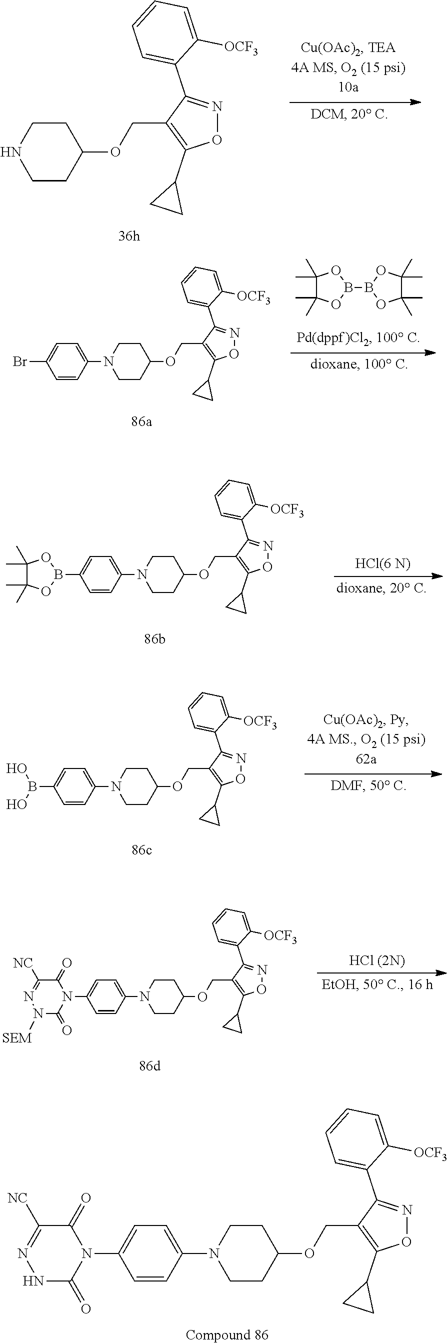

4-(((1-(4-bromophenyl)piperidin-4-yl)oxy)methyl)-5-cyclopropyl-3-(2-(trifluoromethoxy)phenyl)isoxazole (86a). To a solution of (4-bromophenyl)boronic acid (10a) (275.73 mg, 1.37 mmol) and 5-cyclopropyl-4-((piperidin-4-yloxy)methyl)-3-(2-(trifluoromethoxy)phenyl)isoxazole (36h) (350 mg, 915.33 umol) in DCM (7 mL) was added Cu(OAc)₂ (199.50 mg, 1.10 mmol), TEA (185.24 mg, 1.83 mmol) and Molecular sieve 4 A (70 mg) at 20° C. and the mixture was stirred at 20° C. for 16 hours under O₂ atmosphere. The reaction mixture was diluted with DCM (30 mL) and filtered and the filtrate was washed with H₂O (10 mL), brine (10 mL), dried over Na₂SO₄ and filtered. The filtrate was concentrated under reduced pressure to give a residue. The residue was purified by column chromatography (SiO₂, petroleum ether:ethyl acetate=50:1 to 5:1) to give 86a. MS mass calculated for [M+H]⁺(C₂₅H₂₄BrF₃N₂O₃) requires m/z, 537.0, LCMS found m/z, 537.1; ¹H NMR (400 MHz, CHLOROFORM-d) δ=7.58 (dd, J=1.7, 7.8 Hz, 1H), 7.54-7.47 (m, 1H), 7.42-7.35 (m, 2H), 7.34-7.29 (m, 2H), 6.78-6.72 (m, 2H), 4.40 (s, 2H), 3.42 (tt, J=3.8, 8.0 Hz, 1H), 3.36-3.27 (m, 2H), 2.84 (ddd, J=3.3, 9.1, 12.4 Hz, 2H), 2.15 (tt, J=5.1, 8.4 Hz, 1H), 1.88-1.80 (m, 2H), 1.65-1.56 (m, 2H), 1.28-1.21 (m, 2H), 1.15-1.07 (m, 2H).

5-cyclopropyl-4-(((1-(4-(4,4,5,5-tetramethyl-1,3,2-dioxaborolan-2-yl)phenyl)piperidin-4-yl)oxy)methyl)-3-(2-(trifluoromethoxy)phenyl)isoxazole (86b). To a solution of 4-(((1-(4-bromophenyl)piperidin-4-yl)oxy)methyl)-5-cyclopropyl-3-(2-(trifluoromethoxy)phenyl)isoxazole (86a) (400 mg, 744.37 umol) in dioxane (16 mL) was added 4,4,5,5-tetramethyl-2-(4,4,5,5-tetramethyl-1,3,2-dioxaborolan-2-yl)-1,3,2-dioxaborolane (567.07 mg, 2.23 mmol), Pd(dppf)Cl₂ (54.47 mg, 74.44 umol) and KOAc (146.10 mg, 1.49 mmol) at 20° C. and the mixture was heated to 100° C. for 16 hours. The reaction mixture was cooled to 45° C., then ethyl acetate (20 mL) and 3-mercaptopropyl-functionalized silica gel (500 mg) were added. The mixture was stirred at 45° C. for 2 hours and was filtered. The filtrate was concentrated under reduced pressure to give a residue. The residue was purified by column chromatography (SiO₂, petroleum ether:ethyl acetate=50:1 to 3:1) to give 86b. MS mass calculated for [M+H]⁺(C₃₁H₃₆BF₃N₂O₅) requires m/z, 585.2, LCMS found m/z, 584.3, 585.3.

(4-(4-((5-cyclopropyl-3-(2-(trifluoromethoxy)phenyl)isoxazol-4-yl)methoxy)piperidin-1-yl)phenyl)boronic acid (86c). To a solution of 5-cyclopropyl-4-(((1-(4-(4,4,5,5-tetramethyl-1,3,2-dioxaborolan-2-yl)phenyl)piperidin-4-yl)oxy)methyl)-3-(2-(trifluoromethoxy)phenyl)isoxazole (86b) (300 mg, 513.32 umol) in dioxane (2.5 mL) was added aqueous HCl (6 M, 2.5 mL) at 20° C. and the mixture was stirred for 1 hour. The reaction mixture was purified by prep-HPLC (column: Phenomenex Gemini-NX C18 75*30 mm*3 um; mobile phase: [water (10 mM NH₄HCO₃)-ACN]; B %: 40%-70%, 6 min) to give Compound 86c. MS mass calculated for [M+H]⁺(C₂₅H₂₆BF₃N₂O₅) requires m/z, 503.1, LCMS found m/z, 502.2, 503.2; ¹H NMR (400 MHz, METHANOL-d4) δ=7.59 (d, J=7.6 Hz, 2H), 7.54-7.49 (m, 2H), 7.48-7.44 (m, 2H), 6.88 (br d, J=8.2 Hz, 2H), 4.43 (s, 2H), 3.47 (tt, J=3.8, 8.0 Hz, 1H), 3.40-3.33 (m, 1H), 3.40-3.33 (m, 1H), 2.89 (ddd, J=3.0, 9.1, 12.3 Hz, 2H), 2.32-2.22 (m, 1H), 1.88-1.79 (m, 2H), 1.60-1.48 (m, 2H), 1.18-1.16 (m, 2H), 1.15 (s, 2H).

4-(4-(4-((5-cyclopropyl-3-(2-(trifluoromethoxy)phenyl)isoxazol-4-yl)methoxy)piperidin-1-yl)phenyl)-3,5-dioxo-2-((2-(trimethylsilyl)ethoxy)methyl)-2,3,4,5-tetrahydro-1,2,4-triazine-6-carbonitrile (86d). To a solution of (4-(4-((5-cyclopropyl-3-(2-(trifluoromethoxy)phenyl)isoxazol-4-yl)methoxy)piperidin-1-yl)phenyl)boronic acid (86c) (60 mg, 119.45 umol) and 3,5-dioxo-2-((2-(trimethylsilyl)ethoxy)methyl)-2,3,4,5-tetrahydro-1,2,4-triazine-6-carbonitrile (29.97 mg, 123.16 umol) in DMF (3 mL) were added Cu(OAc)₂ (26.04 mg, 143.34 umol), Molecular sieve 4 A (20 mg) and pyridine (18.90 mg, 238.91 umol) at 20° C. The mixture was heated at 50° C. for 16 hours under O₂ atmosphere. The reaction mixture was poured into H₂O (10 mL) and extracted with DCM (15 mL*3). The organic layer was washed with brine (10 mL), dried over Na₂SO₄, filtered and concentrated under reduced pressure to give a residue. The residue was purified by prep-TLC (SiO₂, dichloromethane:methanol=30:1) to give 86d. MS mass calculated for [M+H]⁺(C₃₅H₃₉F₃N₆O₆Si) requires m/z, 724.2, LCMS found m/z, 725.4.

4-(4-(4-((5-cyclopropyl-3-(2-(trifluoromethoxy)phenyl)isoxazol-4-yl)methoxy)piperidin-1-yl)phenyl)-3,5-dioxo-2,3,4,5-tetrahydro-1,2,4-triazine-6-carbonitrile (Compound 86). To a solution of 4-(4-(4-((5-cyclopropyl-3-(2-(trifluoromethoxy)phenyl)isoxazol-4-yl)methoxy)piperidin-1-yl)phenyl)-3, 5-dioxo-2-((2-(trimethylsilyl)ethoxy)methyl)-2,3,4,5-tetrahydro-1,2,4-triazine-6-carbonitrile (86d) (20 mg, 27.59 umol) in EtOH (0.5 mL) was added HCl (2M, 1 mL) at 20° C. and the mixture was heated to 50° C. for 16 hours. The reaction mixture was concentrated under reduced pressure to a residue. The residue was purified by prep-HPLC (column: Waters Xbridge BEH C18 100*30 mm*10 um; mobile phase: [water (10 mM NH₄HCO₃)-ACN]; B %: 25%-55%, 8 min) to give Compound 86. MS mass calculated for [M+H]⁺(C₂₉H₂₅F₃N₆O₅) requires m/z, 595.1, LCMS found m/z, 595.0; ¹H NMR (400 MHz, CHLOROFORM-d) δ=7.57 (br d, J=7.3 Hz, 1H), 7.54-7.47 (m, 1H), 7.43-7.34 (m, 2H), 7.07 (br d, J=8.8 Hz, 2H), 6.94 (br d, J=9.0 Hz, 2H), 4.41 (s, 2H), 3.53-3.37 (m, 3H), 3.05-2.91 (m, 2H), 2.22-2.10 (m, 1H), 1.83 (br s, 2H), 1.62-1.56 (m, 2H), 1.30-1.21 (m, 2H), 1.17-1.07 (m, 2H).

### Example 87

### 5-(4-(4-((5-cyclopropyl-3-(2,6-dichlorophenyl)isoxazol-4-yl)methoxy)-3,3-difluoropiperidin-1-yl)phenyl)isoxazol-3(2H)-one

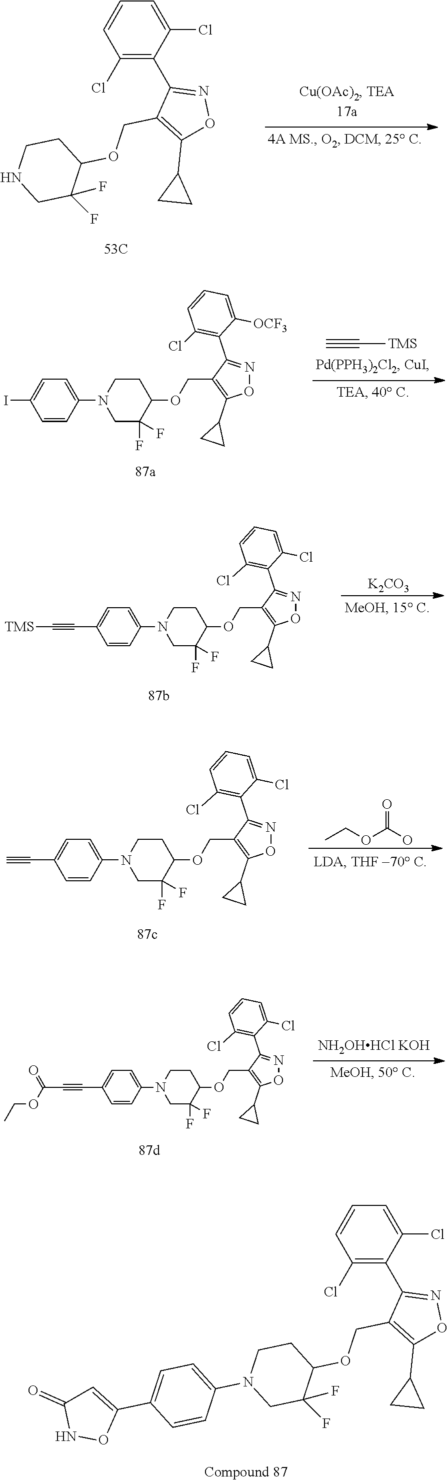

Tert-butyl 5-cyclopropyl-3-(2,6-dichlorophenyl)-4-(((3,3-difluoro-1-(4-iodophenyl)piperidin-4-yl)oxy)methyl)isoxazole (87a). To a solution of 5-cyclopropyl-3-(2,6-dichlorophenyl)-4-(((3,3-difluoropiperidin-4-yl)oxy)methyl)isoxazole (53c) (500 mg, 1.24 mmol) and (4-iodophenyl)boronic acid (17a) (614.57 mg, 2.48 mmol) in dichloromethane (10 mL) was added Cu(OAc)₂ (225.21 mg, 1.24 mmol), TEA (250.93 mg, 2.48 mmol, 345.16 uL) and Molecular sieve 4 A (10 mg). The suspension was degassed and purged with O₂ 3 times and stirred for 18 hours at 25° C. The reaction mixture was filtered through a Celite pad and the filter cake was rinsed with dichloromethane (20 ml*2). The combined filtrate was washed with water (20 mL) and brine (20 mL), dried over anhydrous sodium sulfate, filtered and concentrated. The residue was purified by column chromatography (SiO₂, petroleum ether:ethyl acetate=1:0 to 30:1) to give Compound 87a. MS mass calculated for [M+H]⁺(C₂₄H₂₁Cl₂F₂₁N₂O₂) requires m/z, 605.0/607.0, LCMS found m/z, 605.1/607.1; ¹H NMR (400 MHz, CHLOROFORM-d) δ=8.38 (d, J=2.0 Hz, 1H), 7.64-7.54 (m, 2H), 7.54-7.46 (m, 1H), 7.43-7.33 (m, 2H), 6.57 (d, J=9.3 Hz, 1H), 4.42 (s, 2H), 3.88-3.69 (m, 2H), 3.55 (tt, J=3.5, 7.3 Hz, 1H), 3.43-3.27 (m, 2H), 2.20-2.06 (m, 1H), 1.85-1.66 (m, 2H), 1.56-1.45 (m, 2H), 1.30-1.19 (m, 2H), 1.17-1.02 (m, 2H).

5-cyclopropyl-3-(2,6-dichlorophenyl)-4-(((3,3-difluoro-1-(4-((trimethylsilyl)ethynyl)phenyl)piperidin-4-yl)oxy)methyl)isoxazole (87b). To a solution of 5-cyclopropyl-3-(2,6-dichlorophenyl)-4-(((3,3-difluoro-1-(4-iodophenyl)piperidin-4-yl)oxy)methyl)isoxazole (87a) (150 mg, 247.83 umol and ethynyl(trimethyl)silane (243.42 mg, 2.48 mmol, 343.33 uL) in TEA (2 mL) was added CuI (47.20 mg, 247.83 umol) and Pd(PPh₃)₂Cl₂ (173.96 mg, 247.83 umol) under N₂ in a sealed tube. The resulting mixture was bubbled with N₂ for 10 seconds, then heated to 40° C. and stirred for 20 hours. The reaction mixture was concentrated under reduced pressure and was diluted with ethyl acetate (15 mL). The mixture was washed with water (5 mL) and brine (5 mL), dried over anhydrous sodium sulfate, filtered and concentrated. The residue was purified by prep-TLC (SiO₂, petroleum ether:ethyl acetate=3:1 to give 87b. MS mass calculated for [M+H]⁺(C₂₉H₃OCl₂F₂N₂O₂Si) requires m/z, 575.1/577.1, LCMS found m/z, 575.1/577.1; ¹H NMR (400 MHz, CHLOROFORM-d) δ=7.45-7.27 (m, 5H), 6.79-6.71 (m, 2H), 4.63 (d, J=11.7 Hz, 1H), 4.42 (d, J=11.7 Hz, 1H), 3.76 (br t, J=6.6 Hz, 1H), 3.59 (br dd, J=4.6, 9.0 Hz, 1H), 3.48-3.33 (m, 1H), 3.20-3.13 (m, 1H), 3.07-2.95 (m, 1H), 2.15 (tt, J=5.3, 8.4 Hz, 1H), 1.91-1.81 (m, 1H), 1.77-1.64 (m, 1H), 1.32-1.24 (m, 2H), 1.19-1.09 (m, 2H), 0.34-0.05 (m, 9H).

5-cyclopropyl-3-(2,6-dichlorophenyl)-4-(((1-(4-ethynylphenyl)-3,3-difluoropiperidin-4-yl)oxy)methyl)isoxazole (87c). To a solution of 5-cyclopropyl-3-(2,6-dichlorophenyl)-4-(((3,3-difluoro-1-(4-((trimethylsilyl)ethynyl)phenyl)piperidin-4-yl)oxy)methyl)isoxazole (87b) (230 mg, 399.62 umol) in methanol (5 mL) was added K₂CO₃ (55.23 mg, 399.62 umol). The mixture was stirred at 15° C. for 18 hours and was concentrated under reduced pressure. The residue was purified by prep-TLC (SiO₂, petroleum ether:ethyl acetate=5:1) to give 87c. MS mass calculated for [M+H]⁺(C₂₆H₂₂Cl₂F₂N₂O₂) requires m/z, 503.1/505.1, LCMS found m/z, 503.0/505.0; ¹H NMR (400 MHz, CHLOROFORM-d) δ=7.48-7.34 (m, 4H), 7.34-7.29 (m, 1H), 6.77 (d, J=8.8 Hz, 2H), 4.63 (d, J=11.7 Hz, 1H), 4.42 (d, J=12.2 Hz, 1H), 3.65-3.54 (m, 1H), 3.48-3.35 (m, 1H), 3.33-3.13 (m, 2H), 3.10-3.01 (m, 1H), 3.00 (s, 1H), 2.19-2.10 (m, 1H), 1.87 (ddd, J=4.2, 9.7, 13.6 Hz, 1H), 1.76-1.67 (m, 1H), 1.32-1.26 (m, 2H), 1.15 (qd, J=2.9, 8.4 Hz, 2H).

Ethyl 3-(4-(4-((5-cyclopropyl-3-(2,6-dichlorophenyl)isoxazol-4-yl)methoxy)-3,3-difluoropiperidin-1-yl)phenyl)propiolate (87d). To a solution of 5-cyclopropyl-3-(2,6-dichlorophenyl)-4-(((1-(4-ethynylphenyl)-3,3-difluoropiperidin-4-yl)oxy)methyl)isoxazole (87c) (50 mg, 99.33 umol) in THE (1 mL) was added LDA (1 M in THF, 119.20 uL) dropwise at −70° C., then ethyl carbonochloridate (21.56 mg, 198.66 umol, 18.91 uL) was added dropwise at the same temperature and the reaction mixture was stirred for 1 hour at this temperature. Then LDA (1 M, 119.20 uL) and ethyl carbonochloridate (21.56 mg, 198.66 umol, 18.91 uL) were added dropwise to the mixture at −70° C. and the reaction mixture was stirred for another 2 hours at 15° C. The reaction mixture was cooled to 0° C. and quenched with saturated ammonium chloride solution (5 mL) and extracted with ethyl acetate (10 mL*2). The combined organic phase was washed with brine (10 mL), dried over anhydrous sodium sulfate, filtered and concentrated. The residue was purified by prep-TLC (SiO₂, petroleum ether:ethyl acetate=3:1) to give 87d. MS mass calculated for [M+H]⁺(C₂₉H₂₆Cl₂F₂N₂O₄) requires m/z, 575.1/577.1, LCMS found m/z, 575.0/577.0; ¹H NMR (400 MHz, CHLOROFORM-d) δ=7.47 (d, J=8.6 Hz, 2H), 7.43-7.37 (m, 2H), 7.35-7.29 (m, 1H), 6.79 (d, J=8.8 Hz, 2H), 4.63 (d, J=11.9 Hz, 1H), 4.42 (d, J=11.7 Hz, 1H), 4.29 (q, J=7.2 Hz, 2H), 3.61 (br d, J=5.3 Hz, 1H), 3.56-3.45 (m, 1H), 3.30 (br d, J=11.9 Hz, 2H), 3.15-3.04 (m, 1H), 2.20-2.09 (m, 1H), 1.86 (br s, 1H), 1.70 (br dd, J=4.3, 10.0 Hz, 1H), 1.35 (t, J=7.2 Hz, 3H), 1.28 (br s, 2H), 1.15 (br dd, J=3.1, 8.4 Hz, 2H).

5-(4-(4-((5-cyclopropyl-3-(2,6-dichlorophenyl)isoxazol-4-yl)methoxy)-3,3-difluoropiperidin-1-yl)phenyl)isoxazol-3(2H)-one (Compound 87). To a solution of ethyl 3-(4-(4-((5-cyclopropyl-3-(2,6-dichlorophenyl)isoxazol-4-yl)methoxy)-3,3-difluoropiperidin-1-yl)phenyl)propiolate (87d) (20 mg, 34.76 umol) in methanol (1 mL) was added hydroxylamine hydrochloride (24.15 mg, 347.57 umol) and KOH (35.10 mg, 625.62 umol). The mixture was heated to 50° C. and stirred for 5 hours. The reaction mixture was filtered and the filtrate was concentrated under reduced pressure. The residue was purified by prep-HPLC (neutral condition; column: Phenomenex Gemini-NX C18 75*30 mm*3 um; mobile phase: [water (10 mM NH₄HCO₃)-ACN]; B %: 27%-47%, 6 min) to give Compound 87. MS mass calculated for [M+H]⁺(C₂₇H₂₃Cl₂F₂N₃O₄) requires m/z, 562.1/564.1, LCMS found m/z, 562.0/564.0; ¹H NMR (400 MHz, CHLOROFORM-d) δ=7.61 (d, J=8.6 Hz, 2H), 7.45-7.37 (m, 2H), 7.36-7.30 (m, 1H), 6.89 (d, J=8.8 Hz, 2H), 6.05 (s, 1H), 4.64 (d, J=11.7 Hz, 1H), 4.43 (d, J=11.9 Hz, 1H), 3.62 (br s, 1H), 3.55-3.46 (m, 1H), 3.38-3.24 (m, 2H), 3.15-3.06 (m, 1H), 2.19-2.11 (m, 1H), 1.95-1.83 (m, 1H), 1.74 (br s, 1H), 1.29 (br s, 2H), 1.16 (br dd, J=2.9, 8.4 Hz, 2H).

### Example 88

### 2-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)-1,2,4-triazine-3, 5(2H,4H)-dione

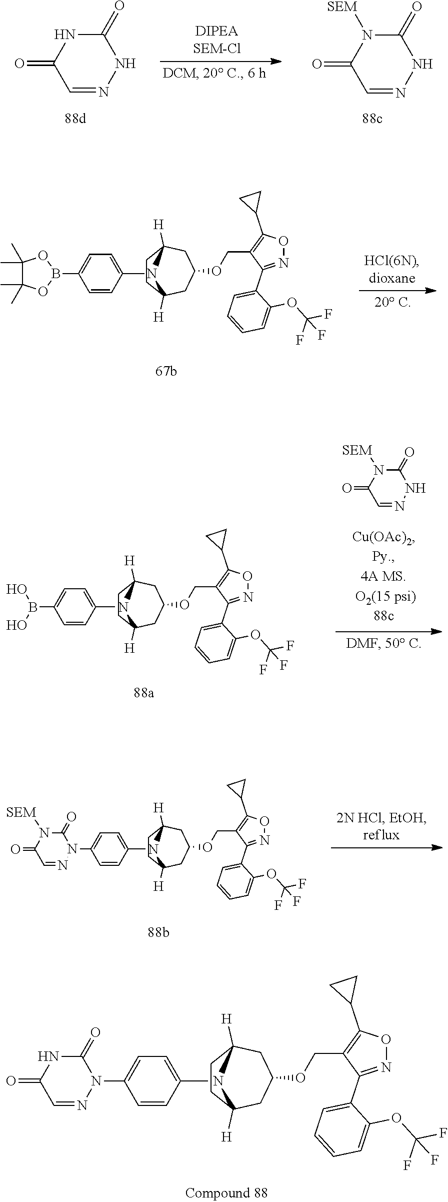

4-((2-(trimethylsilyl)ethoxy)methyl)-1,2,4-triazine-3,5(2H,4H)-dione(88c). To a solution of 2H-1,2,4-triazine-3,5-dione (1 g, 8.84 mmol) in DCM (10 mL) was added DIEA (3.43 g, 26.53 mmol, 4.62 mL) and SEM-Cl (1.47 g, 8.84 mmol, 1.57 mL) at 20° C., and the mixture was stirred at 20° C. for 2 hours. The reaction mixture was poured into H₂O (20 mL) and extracted with DCM (20 mL*2). The combined organic layers were washed with brine (10 mL*2), dried over Na₂SO₄, filtered and concentrated under reduced pressure to give a residue. The residue was purified by column chromatography (SiO₂, petroleum ether/ethyl acetate=50/1 to 5/1) and prep-TLC (SiO₂, DCM:MeOH=20:1) to give 88c. MS mass calculated for [M−H]⁻ (C₉H₁₇N₃O₃Si) requires m/z, 244.1, LCMS found m/z, 244.1; ¹H NMR (400 MHz, CHLOROFORM-d) δ=10.11 (br s, 1H), 7.44 (s, 1H), 5.37 (s, 2H), 3.74-3.67 (m, 2H), 1.01-0.95 (m, 2H), 0.04-−0.02 (m, 9H).

(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)boronic acid (88a). To a solution of 5-cyclopropyl-4-((((1R,3R,5S)-8-(4-(4,4,5,5-tetramethyl-1,3,2-dioxaborolan-2-yl)phenyl)-8-azabicyclo[3.2.1]octan-3-yl)oxy)methyl)-3-(2-(trifluoromethoxy)phenyl)isoxazole (67b) (250 mg, 409.52 umol) in dioxane (2 mL) was added aqueous HCl (6 M, 2 mL) at 20° C. and the mixture was heated to 50° C. for 16 hours. The mixture was purified by prep-HPLC (neutral condition: column: Waters Xbridge Prep OBD C18 150*40 mm*10 um; mobile phase: [water (10 mM NH₄HCO₃)-ACN]; B %: 55%-80%, 8 min) to give 88a. MS mass calculated for [M+H]⁺(C₂₇H₂₅BF₃N₂O₅) requires m/z, 529.2/530.2, LCMS found m/z, 529.1/530.1; ¹H NMR (400 MHz, METHANOL-d4) δ=7.65-7.56 (m, 2H), 7.53-7.46 (m, 4H), 6.78-6.68 (m, 2H), 4.35 (s, 2H), 4.10 (br s, 2H), 3.47-3.42 (m, 1H), 3.43 (br s, 1H), 2.29-2.22 (m, 1H), 2.02-1.79 (m, 6H), 1.56 (br d, J=14.5 Hz, 2H), 1.18-1.13 (m, 4H).

2-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)-4-((2-(trimethylsilyl)ethoxy)methyl)-1,2,4-triazine-3,5(2H,4H)-dione (88b). To a solution of 4-((2-(trimethylsilyl)ethoxy)methyl)-1,2,4-triazine-3,5(2H,4H)-dione (88c) (27.63 mg, 113.57 umol) and (4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)boronic acid (88a) (40 mg, 75.71 umol) in DMF (2 mL) was added Cu(OAc)₂ (13.75 mg, 75.71 umol), 4 A MS (20 mg), Py (11.98 mg, 151.42 umol, 12.22 uL) under O₂ at 20° C. The suspension was degassed under vacuum and purged with O₂ several times. The mixture was stirred under O₂ (15 psi) at 50° C. for 16 hours. Water (5 mL) and ethyl acetate (5 mL) were added to the reaction mixture and the phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, dichloromethane: methanol=50:1, R_{f}=0.50) to give 88b. MS mass calculated for [M+H]⁺(C₃₆H₄₂F₃N₅O₆Si) requires m/z, 726.3/727.3, LCMS found m/z, 726.4/727.3; ¹H NMR (400 MHz, CHLOROFORM-d) δ=7.57-7.51 (m, 3H), 7.42-7.36 (m, 3H), 7.29 (s, 1H), 6.72 (d, J=8.9 Hz, 2H), 5.44 (s, 2H), 4.30 (s, 2H), 4.08 (br s, 2H), 3.77-3.72 (m, 2H), 3.42-3.39 (m, 1H), 2.16-2.09 (m, 1H), 2.01-1.95 (m, 3H), 1.94-1.91 (m, 2H), 1.57 (s, 1H), 1.27-1.22 (m, 4H), 1.13-1.10 (m, 2H), 1.02-0.96 (m, 2H), 0.02 (s, 9H).

2-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)-1,2,4-triazine-3,5(2H,4H)-dione (Compound 88). To a solution of 2-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)-4-((2-(trimethylsilyl)ethoxy)methyl)-1,2,4-triazine-3,5(2H,4H)-dione (88b) (20 mg, 27.55 umol) in ethanol (1 mL) was added aqueous HCl (2N, 2.00 mL) at 20° C., and the mixture was heated to 50° C. for 16 hours. The reaction mixture was concentrated under reduced pressure to remove solvent. The residue was purified by prep-HPLC (neutral condition: column: Waters Xbridge BEH C18 100*25 mm*5 um; mobile phase: [water (10 mM NH₄HCO₃)-ACN]; B %: 35%-70%, 10 min) to give Compound 88. MS mass calculated for [M+H]⁺(C₃₀H₂₈F₃N₅O₅) requires m/z, 596.2/597.2, LCMS found m/z 596.1/597.1; ¹H NMR (400 MHz, CHLOROFORM-d) δ=8.49 (br s, 1H), 7.58-7.49 (m, 3H), 7.40 (t, J=7.1 Hz, 2H), 7.30 (s, 2H), 6.73 (br d, J=8.9 Hz, 2H), 4.30 (s, 2H), 4.08 (br s, 2H), 3.41 (br s, 1H), 2.17-2.08 (m, 1H), 2.03-1.84 (m, 6H), 1.57-1.52 (m, 2H), 1.28-1.21 (m, 2H), 1.16-1.09 (m, 2H).

### Example 89

### 2-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)-3, 5-dioxo-2,3,4,5-tetrahydro-1,2,4-triazine-6-carbonitrile

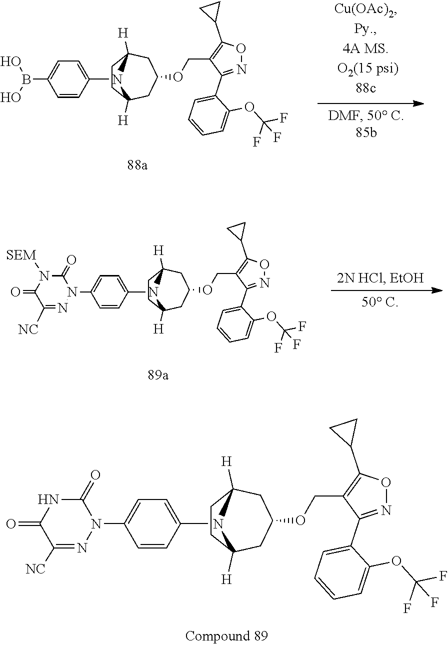

2-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)-3,5-dioxo-4-((2-(trimethylsilyl)ethoxy)methyl)-2,3,4,5-tetrahydro-1,2,4-triazine-6-carbonitrile (89a). To a solution of 3,5-dioxo-4-((2-(trimethylsilyl)ethoxy)methyl)-2,3,4,5-tetrahydro-1,2,4-triazine-6-carbonitrile (2) (30.47 mg, 113.57 umol) and (4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)boronic acid (88a) (40 mg, 75.71 umol) in DMF (2 mL) was added Cu(OAc)₂ (13.75 mg, 75.71 umol), 4 A MS (20 mg) and pyridine (11.98 mg, 151.42 umol, 12.22 uL) under O₂ at 20° C. The suspension was degassed under vacuum and purged with O₂ several times. The mixture was stirred under O₂ (15 psi) at 50° C. for 16 hours. The reaction mixture was concentrated under reduced pressure to remove solvent. Water (5 mL) and ethyl acetate (5 mL) were added into the reaction mixture and the phases were separated. The aqueous phase was extracted with ethyl acetate (5 mL*4). The combined organic phase was washed with brine (20 mL*2), dried with anhydrous Na₂SO₄, filtered and concentrated under reduced pressure. The residue was purified by prep-TLC (SiO₂, dichloromethane:Methanol=50:1, R_{f}=0.50) to give 89a. MS mass calculated for [M+H]⁺(C₃₇H₄₁F₃N₆O₆Si) requires m/z, 751.3/752.3, LCMS found m/z, 751.3/752.3; ¹H NMR (400 MHz, CHLOROFORM-d) δ=7.58-7.50 (m, 2H), 7.40 (t, J=7.1 Hz, 2H), 7.28 (s, 1H), 7.26 (s, 1H), 6.72 (d, J=9.0 Hz, 2H), 5.45 (s, 2H), 4.33-4.29 (m, 2H), 4.09 (br s, 2H), 3.78-3.71 (m, 2H), 3.42 (br s, 1H), 2.16-2.09 (m, 1H), 1.99-1.94 (m, 3H), 1.92-1.87 (m, 2H), 1.59 (s, 1H), 1.28-1.22 (m, 1H), 1.28-1.21 (m, 3H), 1.15-1.09 (m, 2H), 1.02-0.96 (m, 2H), 0.04-−0.01 (m, 9H).

2-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)-3,5-dioxo-2,3,4,5-tetrahydro-1,2,4-triazine-6-carbonitrile (Compound 89). To a solution of 2-(4-((1R,3R,5S)-3-((5-cyclopropyl-3-(2-(trifluoromethoxy)phenyl)isoxazol-4-yl)methoxy)-8-azabicyclo[3.2.1]octan-8-yl)phenyl)-3, 5-dioxo-4-((2-(trimethylsilyl)ethoxy)methyl)-2,3,4,5-tetrahydro-1,2,4-triazine-6-carbonitrile (89a) (20 mg, 26.64 umol) in ethanol (1 mL) was added aqueous HCl (2 M, 2.00 mL) at 20° C., and the mixture was heated to 50° C. for 16 hours. The reaction mixture was concentrated under reduced pressure to remove solvent. The residue was purified by prep-HPLC (neutral condition: column: Waters Xbridge BEH C18 100*25 mm*5 um; mobile phase: [water (10 mM NH₄HCO₃)-ACN]; B %: 30%-60%, 10 min) to give Compound 89. MS mass calculated for [M+H]⁺(C₃₁H₂₇F₃N₆O₅) requires m/z, 621.2/622.2, LCMS found m/z 621.2/622.2; ¹H NMR (400 MHz, CHLOROFORM-d) δ=60-7.50 (m, 2H), 7.41 (t, J=7.1 Hz, 2H), 7.26 (s, 2H), 6.72 (d, J=9.1 Hz, 2H), 4.32 (s, 2H), 4.09 (br s, 2H), 3.43 (br s, 1H), 2.18-2.10 (m, 1H), 2.02-1.84 (m, 6H), 1.58 (br d, J=14.3 Hz, 2H), 1.29-1.22 (m, 2H), 1.17-1.10 (m, 2H).

### Biological Examples

### Example B1. FXR Cellular Assay and FXR Biochemical Assay

A cell based FXR assay was used to detect protein-protein interactions between an activated (ligand-bound), full length human FXR protein and a nuclear fusion protein containing a Steroid Receptor Coactivator Peptide (SCRP) domain. In this approach, two weakly complementing fragments of the 3-gal enzyme were expressed within stably transfected Chinese Hamster Ovary (CHO-K₁) cells. The complementing fragments of the 3-gal enzyme (ProLink, PK; and Enzyme Acceptor, EA) were translationally fused to the C-terminus of a full-length FXR and to the SRCP, respectively. Complementation was driven by the protein-protein interaction between SRCP-EA and a ProLink-labeled FXR. Upon FXR-SRCP binding, the two fragments of 3-gal complement, forming a functional enzyme capable of hydrolyzing a substrate molecule and generating a chemiluminescent signal.

CHO-K₁ cells containing SRCP-EA and ProLink-labeled FXR were plated in 384-well microplates and incubated at 37° C. overnight. A positive control FXR agonist, GW4064 (0-10 micromolar), or test compounds (0-10 micromolar) were added to plated cells and incubated at 37° C. for 6h (final DMSO vehicle concentration was 1%). The assay signal was generated by addition of 50% (v/v) detection reagent (19:5:1, cell assay buffer: substrate reagent 1: substrate reagent 2, DiscoverX) followed by incubation for 1h at ambient room temperature. Microplates were analyzed using an Envision reader (PerkinElmer Life and Analytical Sciences, Signal=chemiluminescence). EC₅₀ values were calculated using non-linear regression curve fitting in GraphPad Prism and are shown in Table 2 below.

A cell free coactivator recruitment assay was developed for FXR, to measure the functional potency of novel FXR compounds. In this assay, novel FXR compounds were bound to the ligand binding domain (LBD) of recombinant human FXR. The ability of this liganded homodimeric complex to recruit the co-activator protein steroid receptor coactivator 1 (SRC-1) was measured using Time-Resolved Fluorescence Resonance Energy Transfer (TR-FRET).

The LBD of human FXR (amino acids 244-476) was cloned into an expression vector for protein expression in SF9 insect cells. Purified FXR LBD was incubated in TR-FRET coregulator buffer G (Eurofins) with test compounds (0-10 micromolar) and a labeled nuclear receptor interaction domain of SRC-1 at 25° C. for 60 minutes (final DMSO vehicle concentration was 1.2%). Assays were analyzed using an Envision TR-FRET reader (PerkinElmer Life and Analytical Sciences). Compound activity at each concentration was defined as a percentage of the maximal activity of the natural FXR ligand agonist chenodeoxycholic acid (CDCA) generating a dose response curve. EC50 values were calculated using non-linear regression curve fitting in GraphPad Prism and are shown in Table 2 below.

TABLE 2

| | EC₅₀ FXR-cell | EC₅₀ FXR- |
|---|---|---|
| Example | Luminescence/ | Biochemical |
| No. | μM | TR-FRET/μM |
| 1 | 1.125 | 3.184 |
| 2 | 0.6912 | 2.885 |
| 3 | 0.0970 | 0.0036 |
| | 0.0498 | 0.0029 |
| 4 | 1.467 | 0.2197 |
| 5 | 2.53 | 0.031 |
| 6 | 0.3724 | 0.0044 |
| 7 | 1.231 | 0.1046 |
| 8 | 2.108 | 0.0281 |
| 9 | 1.595 | 0.1746 |
| 10 | 0.4086 | 0.0091 |
| 11 | 0.2801 | 0.0219 |
| 12 | 0.4371 | 0.0737 |
| 13 | 0.0823 | 0.0035 |
| 14 | 0.064 | 0.0085 |
| 15 | 5.514 | 0.8924 |
| 16 | 0.1954 | 0.024 |
| 17 | 4.734 | 6.067 |
| 18 | 0.0184 | 0.0029 |
| 19 | 0.8395 | 0.1221 |
| 20 | 0.3975 | 0.1135 |
| 21 | 0.3434 | 0.0793 |
| 22 | 0.418 | 0.0633 |
| 23 | 0.0277 | 0.0031 |
| 24 | 2.3136 | 0.5515 |
| 25 | 0.7793 | 0.0091 |
| 26 | 0.5618 | 0.1112 |
| 27 | 0.9597 | 0.2268 |
| 28 | 1.608 | 0.1495 |
| 29 | 3.1904 | 0.2307 |
| 30 | 3.6206 | 0.2963 |
| 31 | 0.2712 | 0.045 |
| 32 | 0.1946 | 0.0126 |
| 33 | 0.7016 | 0.1418 |
| 34 | 0.7847 | 0.1534 |
| 35 | 0.3238 | 0.0435 |
| 36 | 0.1125 | 0.0188 |
| 37 | 0.2745 | 0.0494 |
| 38 | 0.2632 | 0.0286 |
| 39 | 0.1544 | 0.0161 |
| 40 | 0.3407 | 0.076 |
| 41 | 0.2442 | 0.0584 |
| 42 | 10 | 0.1393 |
| 43 | 0.2482 | 0.0112 |
| 44 | 0.3705 | 0.0213 |
| 45 | 10 | 0.6096 |
| 46 | 10 | 0.0746 |
| 47 | 0.0767 | 0.0037 |
| 48 | 0.3359 | 0.0416 |
| 49 | 0.1492 | 0.0117 |
| 50 | 0.0538 | 0.0032 |
| 51 | 1.292 | 0.325 |
| 52 | 1.626 | 0.143 |
| 53 | 0.0943 | 0.009 |
| 54 | 0.7145 | 0.0376 |
| 55 | 0.8524 | 0.1744 |
| 56 | 0.8871 | 0.0431 |
| 57 | 0.4973 | 0.0847 |
| 58 | 11.66 | 0.4206 |
| 59 | 0.264 | 0.0179 |
| 60 | 0.7794 | 0.1041 |
| 61 | 1.921 | 0.2981 |
| 62 | >10 | 6.348 |
| | 4.734 | 6.067 |
| 63 | 2.658 | 0.2149 |
| 64 | 2.308 | 0.07 |
| 65 | 0.9543 | 0.0221 |
| 66 | 0.2471 | 0.003 |
| 67 | 0.0657 | 0.0127 |
| 68 | 0.1681 | 0.0264 |
| 69 | 0.0576 | 0.0095 |
| 70 | >10 | 0.2331 |
| 71 | 0.0247 | 0.0021 |
| 72 | 0.0549 | 0.0045 |
| 73 | 0.3501 | 0.0476 |
| 74 | 1.45 | 0.1069 |
| 75 | 0.3823 | ND |
| 76 | 0.1958 | ND |
| 77 | 0.0695 | ND |
| 78 | 0.1538 | ND |
| 79 | 0.3117 | ND |
| 80 | 0.6415 | ND |
| 81 | 0.1745 | ND |
| 82 | 1.831 | ND |
| 83 | 5.249 | ND |
| 84 | 1.534 | ND |
| 85 | 0.7548 | ND |
| 86 | 22.93 | ND |
| 87 | 0.2612 | ND |
| 88 | 0.4604 | ND |
| 89 | 1.0023 | ND |

ND: not determined

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

Although the foregoing disclosure has been described in some detail by way of illustration and example for purposes of clarity of understanding, it is apparent to those skilled in the art that certain minor changes and modifications will be practiced in light of the above teaching. Therefore, the description and examples should not be construed as limiting the scope of the disclosure.

1. A compound of formula (I): 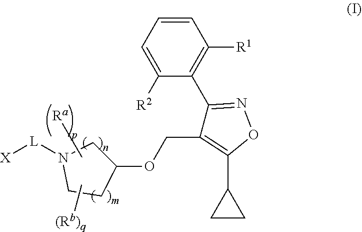
or a stereoisomer, tautomer, or a pharmaceutically acceptable salt of any of the foregoing,
wherein:
R¹ and R² are independently hydrogen, halogen, C₁-C₆ alkyl, or C₁-C₆ alkoxy, wherein the C₁-C₆ alkyl and C₁-C₆ alkoxy are optionally substituted by one to three halogen;
m is 0, 1 or 2;
n is 1 or 2;
p is 0, 1 or 2;
q is 0, 1 or 2;
R^{a} and R^{b} are independently halogen or C₁-C₆ alkyl,
or p and q are both 1, and R^{a} and R^{b} are taken together with the carbon atoms to which they are attached to form a C₄-C₆ bridge;
L is —C(═O)—, phenylene, or 5- or 6-membered heteroarylene, wherein the phenylene and 5- or 6-membered heteroarylene are optionally substituted by one to three substituents each independently selected from the group consisting of C₁-C₆ alkyl, C₁-C₆ alkoxy, halogen, and cyano; and
X is 3- to 6-membered heterocyclyl or 3- to 6-membered heteroaryl each containing one to four annular heteroatoms selected from the group consisting of N, O and S, wherein the 3- to 6-membered heterocyclyl and 3- to 6-membered heteroaryl are optionally substituted by one to three substituents each independently selected from the group consisting of halogen, cyano and oxo.

2. The compound of claim 1, or a stereoisomer, tautomer, or a pharmaceutically acceptable salt of any of the foregoing, wherein R¹ and R² are independently hydrogen, chloro, fluoro, methoxy, ethoxy, or trifluoromethoxy.

3. The compound of claim 1, or a stereoisomer, tautomer, or a pharmaceutically acceptable salt of any of the foregoing, wherein at least one of R¹ and R² is not hydrogen.

4. The compound of claim 1, or a stereoisomer, tautomer, or a pharmaceutically acceptable salt of any of the foregoing, wherein one of R¹ and R² is chloro, fluoro, methoxy or trifluoromethoxy and the other is hydrogen.

5. The compound of any one of claims 1-4, or a stereoisomer, tautomer, or a pharmaceutically acceptable salt of any of the foregoing, wherein m is 1 and n is 2.

6. The compound of any one of claims 1-4, or a stereoisomer, tautomer, or a pharmaceutically acceptable salt of any of the foregoing, wherein m is 2 and n is 2.

7. The compound of any one of claims 1-4, or a stereoisomer, tautomer, or a pharmaceutically acceptable salt of any of the foregoing, wherein m is 0 and n is 1.

8. The compound of any one of claims 1-7, or a stereoisomer, tautomer, or a pharmaceutically acceptable salt of any of the foregoing, wherein R^{a} and R^{b} are independently halogen.

9. The compound of any one of claims 1-7, or a stereoisomer, tautomer, or a pharmaceutically acceptable salt of any of the foregoing, wherein R^{a} and R^{b} are independently chloro, fluoro, or methyl.

10. The compound of any one of claims 1-7, or a stereoisomer, tautomer, or a pharmaceutically acceptable salt of any of the foregoing, wherein p and q are both 0.

11. The compound of any one of claims 1-9, or a stereoisomer, tautomer, or a pharmaceutically acceptable salt of any of the foregoing, wherein p and q are both 1.

12. The compound of any one of claims 1-9, or a stereoisomer, tautomer, or a pharmaceutically acceptable salt of any of the foregoing, wherein one of p and q is 0, and the other is 2.

13. The compound of any one of claims 1-9, or a stereoisomer, tautomer, or a pharmaceutically acceptable salt of any of the foregoing, wherein p and q are both 2.

14. The compound of any one of claims 1-7, or a stereoisomer, tautomer, or a pharmaceutically acceptable salt of any of the foregoing, wherein p and q are both 1, and R^{a} and R^{b} are taken together with the carbon atoms to which they are attached to form a C₄-C₆ bridge.

15. The compound of claim 14, or a stereoisomer, tautomer, or a pharmaceutically acceptable salt of any of the foregoing, wherein R^{a} and R^{b} are taken together with the carbon atoms to which they are attached to form a C₄ bridge.

16. The compound of claim 15, or a stereoisomer, tautomer, or a pharmaceutically acceptable salt of any of the foregoing, wherein the 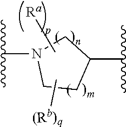
moiety is

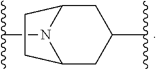 [SMILES: *C1CC2CCC(C1)N2*]

17. The compound of any one of claims 1-16, or a stereoisomer, tautomer, or a pharmaceutically acceptable salt of any of the foregoing, wherein L is —C(═O)—.

18. The compound of any one of claims 1-16, or a stereoisomer, tautomer, or a pharmaceutically acceptable salt of any of the foregoing, wherein L is phenylene or 5- or 6-membered heteroarylene, and wherein the phenylene and 5- or 6-membered heteroarylene are optionally substituted by one to three substituents each independently selected from the group consisting of C₁-C₆ alkyl, C₁-C₆ alkoxy, halogen, and cyano.

19. The compound of claim 18, or a stereoisomer, tautomer, or a pharmaceutically acceptable salt of any of the foregoing, wherein L is phenylene optionally substituted by one to three substituents each independently selected from the group consisting of methyl, chloro and fluoro.

20. The compound of claim 19, or a stereoisomer, tautomer, or a pharmaceutically acceptable salt of any of the foregoing, wherein L is phenylene.

21. The compound of claim 18, or a stereoisomer, tautomer, or a pharmaceutically acceptable salt of any of the foregoing, wherein L is 5- or 6-membered heteroarylene optionally substituted by one to three substituents each independently selected from the group consisting of C₁-C₆ alkyl, C₁-C₆ alkoxy, halogen, and cyano.

22. The compound of claim 21, or a stereoisomer, tautomer, or a pharmaceutically acceptable salt of any of the foregoing, wherein L is 5-membered heteroarylene optionally substituted by one to three substituents each independently selected from the group consisting of C₁-C₆ alkyl, C₁-C₆ alkoxy, halogen, and cyano.

23. The compound of claim 21, or a stereoisomer, tautomer, or a pharmaceutically acceptable salt of any of the foregoing, wherein L is 6-membered heteroarylene optionally substituted by one to three substituents each independently selected from the group consisting of C₁-C₆ alkyl, C₁-C₆ alkoxy, halogen, and cyano.

24. The compound of claim 21, or a stereoisomer, tautomer, or a pharmaceutically acceptable salt of any of the foregoing, wherein L is 5- or 6-membered heteroarylene selected from the group consisting of 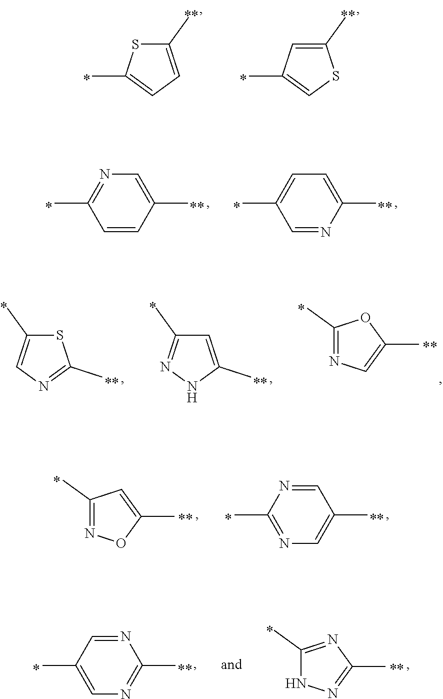
wherein
* represents the point of attachment to the remainder of the molecule via the nitrogen,
** represents the point of attachment to the X moiety, and
each of which is optionally substituted by methyl, chloro or fluoro.

25. The compound of any one of claims 1-24, or a stereoisomer, tautomer, or a pharmaceutically acceptable salt of any of the foregoing, wherein X is 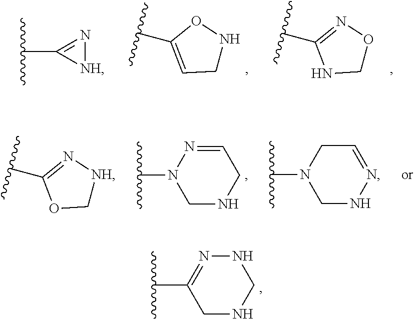
each of which is optionally substituted by one to three substituents each independently selected from the group consisting of cyano and oxo.

26. The compound ofany one of claims 1-24, or a stereoisomer, tautomer, or a pharmaceutically acceptable salt of any of the foregoing, wherein X is

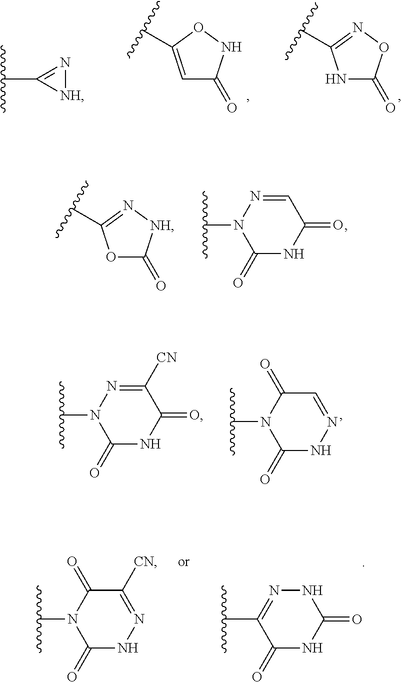

27. The compound of claim 1, or a stereoisomer, tautomer, or a pharmaceutically acceptable salt of any of the foregoing, wherein the compound is selected from the group consisting of the compounds in Table 1.

28. A pharmaceutical composition comprising the compound of any one of claims 1-27, or a stereoisomer, tautomer, or a pharmaceutically acceptable salt of any of the foregoing, and a pharmaceutical acceptable excipient.

29. A method of treating a disease mediated by farnesoid X receptor (FXR) in an individual in need thereof, comprising administering to the individual a therapeutically effective amount of the compound of any one of claims 1-27, or a stereoisomer, tautomer, or a pharmaceutically acceptable salt of any of the foregoing, or the pharmaceutical composition of claim 28.

30. The method of claim 29, wherein the disease is a liver disease.

31. The method of claim 30, wherein the liver disease is primary biliary cirrhosis (PBC), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, viral hepatitis, alcoholic liver disease, nonalcoholic fatty liver disease (NAFLD), nonalcoholic steatohepatitis (NASH), graft versus host disease, transplant liver regeneration, congenital hepatic fibrosis, choledocholithiasis, granulomatous liver disease, intra- or extrahepatic malignancy, Sjogren's syndrome, sarcoidosis, Wilson's disease, Gaucher's disease, hemochromatosis, or oti-antitrypsin deficiency.

32. The method of claim 31, wherein the liver disease is NASH.

33. The method of claim 29, wherein the disease is dyslipidemia or a related disease.

34. A compound of any one of claims 1-27, or a stereoisomer, tautomer, or a pharmaceutically acceptable salt of any of the foregoing, for use in the treatment of a disease mediated by FXR.

35. Use of a compound of any one of claims 1-27, or a stereoisomer, tautomer, or a pharmaceutically acceptable salt of any of the foregoing, in the manufacture of a medicament for the treatment of a disease mediated by FXR.
